SEQUENCE LISTING (95 sequences)

SEQ ID NO: 1 - protein, 7 aa, Simian virus 40
PKKKRKV

SEQ ID NO: 2 - protein, 10 aa, Simian virus 40
PKKKRKVEAS

SEQ ID NO: 3 - protein, 16 aa, Artificial Sequence
  Synthetic
KRPAATKKAGQAKKKK

SEQ ID NO: 4 - protein, 9 aa, Homo sapiens
PAAKRVKLD

SEQ ID NO: 5 - protein, 11 aa, Homo sapiens
RQRRNELKRSP

SEQ ID NO: 6 - protein, 38 aa, Homo sapiens
NQSSNFGPMKGGNFGGRSSGPYGGGGQYFAKPRNQGGY

SEQ ID NO: 7 - protein, 42 aa, Homo sapiens
RMRIZFKNKGKDTAELRRRRVEVSVELRKAKKDEQILKRRNV

SEQ ID NO: 8 - protein, 8 aa, Homo sapiens
VSRKRPRP

SEQ ID NO: 9 - protein, 8 aa, Homo sapiens
PPKKARED

SEQ ID NO: 10 - protein, 8 aa, Homo sapiens
PQPKKKPL

SEQ ID NO: 11 - protein, 12 aa, Mus sp.
SALIKKKKKMAP

SEQ ID NO: 12 - protein, 5 aa, Influenza virus
DRLRR

SEQ ID NO: 13 - protein, 7 aa, Influenza virus
PKQKKRK

SEQ ID NO: 14 - protein, 10 aa, Hepatitis D virus
RKLKKKIKKL

SEQ ID NO: 15 - protein, 10 aa, Mus sp.
REKKKFLKRR

SEQ ID NO: 16 - protein, 20 aa, Homo sapiens
KRKGDEVDGVDEVAKKKSKK

SEQ ID NO: 17 - protein, 17 aa, Homo sapiens
RKCLQAGMNLEARKTKK

SEQ ID NO: 18 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 19 - protein, 9 aa, Homo sapiens
IEVMYPPPY

SEQ ID NO: 20 - DNA, 20 bp, Artificial Sequence
  Synthetic
gcggggtccgcatggcggcg

SEQ ID NO: 21 - DNA, 20 bp, Artificial Sequence
  Synthetic
cagaatacaactcaagcagg

SEQ ID NO: 22 - DNA, 20 bp, Artificial Sequence
  Synthetic
gaatggatgctgcagatgcg

SEQ ID NO: 23 - DNA, 20 bp, Artificial Sequence
  Synthetic
ctgaccttcggtgaaatcgg

SEQ ID NO: 24 - DNA, 20 bp, Artificial Sequence
  Synthetic
tcagtttaggtatctagtgt

SEQ ID NO: 25 - DNA, 20 bp, Artificial Sequence
  Synthetic
ctatacacagctttattcct

SEQ ID NO: 26 - DNA, 20 bp, Artificial Sequence
  Synthetic
catggaaagagctcgagccc

SEQ ID NO: 27 - DNA, 20 bp, Artificial Sequence
  Synthetic
cactcagagcctccgaagac

SEQ ID NO: 28 - DNA, 20 bp, Artificial Sequence
  Synthetic
cggagccgccgttacgtaac

SEQ ID NO: 29 - DNA, 20 bp, Artificial Sequence
  Synthetic
cccctcctcgagcgtgggga

SEQ ID NO: 30 - DNA, 20 bp, Artificial Sequence
  Synthetic
aggcggctcgcgtcactgtc

SEQ ID NO: 31 - DNA, 20 bp, Artificial Sequence
  Synthetic
gcgcgtgtccttgtaaacag

SEQ ID NO: 32 - DNA, 20 bp, Artificial Sequence
  Synthetic
gccgcagggagcgcgggccc

SEQ ID NO: 33 - DNA, 20 bp, Artificial Sequence
  Synthetic
gtgggtcctggtaccgggtg

SEQ ID NO: 34 - DNA, 20 bp, Artificial Sequence
  Synthetic
cggaaccctcccaaaacttg

SEQ ID NO: 35 - DNA, 20 bp, Artificial Sequence
  Synthetic
agcagctggcgcgtcccgcg

SEQ ID NO: 36 - DNA, 20 bp, Artificial Sequence
  Synthetic
tcgggaagctgcgcagaact

SEQ ID NO: 37 - DNA, 20 bp, Artificial Sequence
  Synthetic
agcttccggagggttgcgca

SEQ ID NO: 38 - DNA, 20 bp, Artificial Sequence
  Synthetic
cctttatcacggttttaggg

SEQ ID NO: 39 - DNA, 20 bp, Artificial Sequence
  Synthetic
ctgggaccttgagagcggcc

SEQ ID NO: 40 - DNA, 20 bp, Artificial Sequence
  Synthetic
tatcgcgccagagagggcga

SEQ ID NO: 41 - DNA, 20 bp, Artificial Sequence
  Synthetic
ctgcgcctcactccaggctc

SEQ ID NO: 42 - DNA, 20 bp, Artificial Sequence
  Synthetic
ggagtgaggcgcagcggcag

SEQ ID NO: 43 - DNA, 20 bp, Artificial Sequence
  Synthetic
tacgcacgaaagtgactagg

SEQ ID NO: 44 - DNA, 20 bp, Artificial Sequence
  Synthetic
acatgatgatacatggaggc

SEQ ID NO: 45 - DNA, 20 bp, Artificial Sequence
  Synthetic
ggctcaccttgaagctgttg

SEQ ID NO: 46 - DNA, 20 bp, Artificial Sequence
  Synthetic
tcaagactttgctctccacc

SEQ ID NO: 47 - DNA, 20 bp, Artificial Sequence
  Synthetic
gcggctccggtgcgtccctg

SEQ ID NO: 48 - DNA, 20 bp, Artificial Sequence
  Synthetic
gcgtccctgagtcccagagt

SEQ ID NO: 49 - DNA, 20 bp, Artificial Sequence
  Synthetic
acatgtcagttcatgttcag

SEQ ID NO: 50 - DNA, 20 bp, Artificial Sequence
  Synthetic
ggttcccaaggacctatatg

SEQ ID NO: 51 - DNA, 20 bp, Artificial Sequence
  Synthetic
agtcgctggagattatctct

SEQ ID NO: 52 - DNA, 20 bp, Artificial Sequence
  Synthetic
ccaaaagtcgccctcccggg

SEQ ID NO: 53 - DNA, 20 bp, Artificial Sequence
  Synthetic
ccggagtctcaaagcgcctg

SEQ ID NO: 54 - DNA, 20 bp, Artificial Sequence
  Synthetic
gggtaaaagtactgtcccgg

SEQ ID NO: 55 - DNA, 20 bp, Artificial Sequence
  Synthetic
caacgcagggaaccaaacgg

SEQ ID NO: 56 - DNA, 20 bp, Artificial Sequence
  Synthetic
ggttccagtatgcctcggga

SEQ ID NO: 57 - DNA, 20 bp, Artificial Sequence
  Synthetic
cccatcgatcagcagaactg

SEQ ID NO: 58 - DNA, 20 bp, Artificial Sequence
  Synthetic
gccagcccgagactccgcga

SEQ ID NO: 59 - DNA, 20 bp, Artificial Sequence
  Synthetic
ggggggcctgagctacaaag

SEQ ID NO: 60 - DNA, 20 bp, Artificial Sequence
  Synthetic
tcaactagagctgcggcgcg

SEQ ID NO: 61 - DNA, 20 bp, Artificial Sequence
  Synthetic
ctgaaaaaggaaggagttga

SEQ ID NO: 62 - DNA, 20 bp, Artificial Sequence
  Synthetic
aagatgaaaggaaaggcgtt

SEQ ID NO: 63 - DNA, 44 bp, Artificial Sequence
  Synthetic
ctttccctacacgacgctcttccgatctgggcagcaagggcctc

SEQ ID NO: 64 - DNA, 47 bp, Artificial Sequence
  Synthetic
gactggagttcagacgtgtgctcttccgatctgctctccggccccct

SEQ ID NO: 65 - DNA, 48 bp, Artificial Sequence
  Synthetic
ctttccctacacgacgctcttccgatcttcagggctgaatggatgctg

SEQ ID NO: 66 - DNA, 49 bp, Artificial Sequence
  Synthetic
gactggagttcagacgtgtgctcttccgatcttgccctccccatctccc

SEQ ID NO: 67 - DNA, 44 bp, Artificial Sequence; identical to SEQ ID NO: 63
  Synthetic

SEQ ID NO: 68 - DNA, 47 bp, Artificial Sequence
  Synthetic
gactggagttcagacgtgtgctcttccgatctgcccccacctcgcca

SEQ ID NO: 69 - DNA, 50 bp, Artificial Sequence
  Synthetic
ctttccctacacgacgctcttccgatcttgtttatgtcctagccccatac

SEQ ID NO: 70 - DNA, 53 bp, Artificial Sequence
  Synthetic
gactggagttcagacgtgtgctcttccgatctcttgatggtcactgcttgtcc

SEQ ID NO: 71 - DNA, 53 bp, Artificial Sequence
  Synthetic
ctttccctacacgacgctcttccgatctaaacgctgtgccaattttgtaaatg

SEQ ID NO: 72 - DNA, 55 bp, Artificial Sequence
  Synthetic
gactggagttcagacgtgtgctcttccgatctgacaattagtgcagccaggtcta

SEQ ID NO: 73 - DNA, 48 bp, Artificial Sequence
  Synthetic
ctttccctacacgacgctcttccgatctggagttggtcggggaatctg

SEQ ID NO: 74 - DNA, 52 bp, Artificial Sequence
  Synthetic
gactggagttcagacgtgtgctcttccgatctcaacccgtcgtaaggtctcc

SEQ ID NO: 75 - DNA, 50 bp, Artificial Sequence
  Synthetic
ctttccctacacgacgctcttccgatctaaaagaaacgcggtaatcggac

SEQ ID NO: 76 - DNA, 49 bp, Artificial Sequence
  Synthetic
gactggagttcagacgtgtgctcttccgatctgcgcttccgccaatcac

SEQ ID NO: 77 - DNA, 49 bp, Artificial Sequence
  Synthetic
ctttccctacacgacgctcttccgatctggcctctctctacacgactac

SEQ ID NO: 78 - DNA, 50 bp, Artificial Sequence
  Synthetic
gactggagttcagacgtgtgctcttccgatctcagctccgaagaggcgag

SEQ ID NO: 79 - DNA, 48 bp, Artificial Sequence
  Synthetic
ctttccctacacgacgctcttccgatctattgtccccaacagcaacgg

SEQ ID NO: 80 - DNA, 52 bp, Artificial Sequence
  Synthetic
gactggagttcagacgtgtgctcttccgatctttgtgcagatcgtccagggc

SEQ ID NO: 81 - DNA, 48 bp, Artificial Sequence
  Synthetic
ctttccctacacgacgctcttccgatcttgctggcgattaagtccctc

SEQ ID NO: 82 - DNA, 52 bp, Artificial Sequence
  Synthetic
gactggagttcagacgtgtgctcttccgatctatctgaaaggtcggggtggt

SEQ ID NO: 83 - DNA, 49 bp, Artificial Sequence
  Synthetic
ctttccctacacgacgctcttccgatcttctccaaaagcagtagccaag

SEQ ID NO: 84 - DNA, 52 bp, Artificial Sequence
  Synthetic
gactggagttcagacgtgtgctcttccgatcttatattggagagcctgcccg

SEQ ID NO: 85 - DNA, 47 bp, Artificial Sequence
  Synthetic
ctttccctacacgacgctcttccgatcttcttttctcccagggctgc

SEQ ID NO: 86 - DNA, 51 bp, Artificial Sequence
  Synthetic
gactggagttcagacgtgtgctcttccgatctctttgtagctcaggccccc

SEQ ID NO: 87 - DNA, 44 bp, Artificial Sequence
  Synthetic
ctttccctacacgacgctcttccgatctttcctcgcctgcccct

SEQ ID NO: 88 - DNA, 49 bp, Artificial Sequence
  Synthetic
gactggagttcagacgtgtgctcttccgatctgccgcaggtccaagagg

SEQ ID NO: 89 - DNA, 49 bp, Artificial Sequence
  Synthetic
ctttccctacacgacgctcttccgatctacaaagaggacacgaaggagc

SEQ ID NO: 90 - DNA, 48 bp, Artificial Sequence
  Synthetic
gactggagttcagacgtgtgctcttccgatctccccagcagcagagcg

SEQ ID NO: 91 - DNA, 51 bp, Artificial Sequence
  Synthetic
ctttccctacacgacgctcttccgatcttttcctcccacagttctgctgat

SEQ ID NO: 92 - DNA, 56 bp, Artificial Sequence
  Synthetic
gactggagttcagacgtgtgctcttccgatctcgcagctctagttgaaagaagaca

SEQ ID NO: 93 - DNA, 21 bp, Artificial Sequence
  Synthetic
gggcaggctctccaatataag

SEQ ID NO: 94 - DNA, 24 bp, Artificial Sequence
  Synthetic
  MISC_FEATURE (1)..(1): 5 ′6-FAM = fluorescent dye attached to 5′ end of oligonucelotides
  MISC_FEATURE (9)..(10): ZEN  (N,N-diethyl-4-(4-nitronaphthalen-1- ylazo)-phenylamine) modifier
  MISC_FEATURE (24)..(24): 3′-end modification (3IABkFQ) Iowa Black Quencher oligonucleotide probe label
tgaactactgcgaacctgacctga

SEQ ID NO: 95 - DNA, 23 bp, Artificial Sequence
  Synthetic
ggcatctcaaatacagcagaaag

(19) United States
(12) Patent Application Publication
(10) Pub. No.: US 2024/0261333 A1
(43) Pub. Date: Aug. 8, 2024

(54) NOVEL TARGETS FOR ENHANCING ANTI-TUMOR IMMUNITY

(71) Applicants: THE BROAD INSTITUTE, INC., Cambridge, MA (US); MASSACHUSETTS INSTITUTE OF TECHNOLOGY, Cambridge, MA (US)

(72) Inventors: Julia Joung, Cambridge, MA (US); Feng Zhang, Cambridge, MA (US)

(21) Appl. No.: 18/565,668

(22) PCT Filed: Jun. 3, 2022
(86) PCT No.: PCT/US2022/032109

§ 371 Date: Nov. 30, 2023

Related U.S. Application Data
(60) Provisional application No. 63/196,520, filed on Jun. 3, 2021.

(51) Int. Cl. A61K 35/17; A61K 38/17; A61K 39/00; A61K 45/06; A61K 47/68; A61P 35/00; C07K 14/82; C12N 9/22; C12N 15/11; C12N 15/113; G01N 33/50; G01N 33/574

(52) U.S. Cl. CPC A61K 35/17; A61K 38/1709; A61K 39/4611; A61K 39/4632; A61K 39/464488; A61K 45/06; A61K 47/6815; A61P 35/00; C07K 14/82; C12N 9/22; C12N 15/111; C12N 15/1137; G01N 33/5011; G01N 33/5023; G01N 33/574; C12N 2310/20; C12Y 204/01149

(57) ABSTRACT

The present disclosure relates to identifying novel tumor immune evasion targets. A CRISPR activation screen was employed to identify novel checkpoint inhibitor targets, where upon upregulation, conferred tumor resistance to cytotoxic T cells in model cancer cell lines. Using MAGeCK and FDR analyses to identify candidate genes that were enriched in cancer cells, B3GNT2, MCL1, BCL2A1 and JUNB were identified as the most enriched after a pathway analysis of the top 576 genes prioritized by MAGeCK. Currently, these four genes have not been identified or suggested as possible checkpoint inhibitor targets. Provided herein are methods of targeting the expression or activity of B3GNT2, MCL1, BCL2A1 and JUNB using small molecule agents and/or gene editing methods with the aim of enhancing anti-tumor immunity in subjects in need thereof.

### CROSS-REFERENCE TO RELATED APPLICATIONS

This application claims the benefit of U.S. Provisional Application No. 63/196,520, filed Jun. 3, 2021. The entire contents of the above-identified applications are hereby fully incorporated herein by reference.

### STATEMENT REGARDING FEDERALLY SPONSORED RESEARCH

This invention was made with government support under Grant No.(s) MH117886, HG009761, MH110049, and HL141201 awarded by the National Institutes of Health. The government has certain rights in the invention.

### REFERENCE TO AN ELECTRONIC SEQUENCE LISTING

The contents of the electronic sequence listing (“BROD-4270WP_ST25.txt”; Size is 18,958 bytes and it was created on Jun. 3, 2022) is herein incorporated by reference in its entirety.

### TECHNICAL FIELD

The subject matter disclosed herein is generally directed to methods of enhancing anti-tumor immunity by administering agents that reduce or inhibit the expression or activity of B3GNT2, MCL1, BCL2A1 or JUNB.

### BACKGROUND

The cellular processes that govern tumor resistance to immunotherapy remain poorly understood (Chen and Mellman, 2017; Hegde and Chen, 2020; Zaretsky et al., 2016). By harnessing cytotoxic T cells of the immune system to eliminate cancer cells, cancer immunotherapy has transformed the foundation of cancer treatment and achieved notable clinical successes (Reck et al., 2016). Nevertheless, resistance to immunotherapy is a major challenge (Chen and Mellman, 2017; Hegde and Chen, 2020; Zaretsky et al., 2016), and elucidating the cellular pathways that confer resistance is critical for developing alternative and auxiliary strategies to expand the scope of immunotherapy. Small-scale studies have identified a small number of genes, including CD274 (PD-L1), that enable tumors to evade the immune system, and PD-L1 in particular has been the focus of on-going clinical development (Dong et al., 2002; Holmgaard et al., 2013; Sica et al., 2003; Tseng et al., 2013; Wolchok et al., 2013; Zaretsky et al., 2016). More recently, large-scale, loss-of-function genetic screens using CRISPR have identified additional genes that mediate resistance to T cell-induced cytoxicity in the antigen presentation, interferon-γ (IFNγ)-sensing, tumor necrosis factor (TNF), and autophagy pathways (Lawson et al., 2020; Manguso et al., 2017; Pan et al., 2018; Patel et al., 2017; Vredevoogd et al., 2019). However, in loss-of-function screens, candidate genes that can be inhibited to sensitize tumors against immunotherapy are depleted. As depletion screens have a lower dynamic range than enrichment screens (Doench, 2018), a more tractable approach is to perform a gain-of-function screen to enrich for genes that confer resistance upon upregulation (Decker et al., 2019) and could theoretically be inhibited to sensitize tumors against immunotherapy. Applicants therefore performed a genome-scale CRISPR activation (CRISPRa) screen for resistance against T cell cytotoxicity to gain insight into these processes and identify genes that could potentially improve immunotherapy outcomes.

Citation or identification of any document in this application is not an admission that such a document is available as prior art to the present invention.

### SUMMARY

In certain example embodiments, are disclosed methods of enhancing anti-tumor immunity in a subject in need thereof comprising administering to the subject one or more agents that reduce the expression or activity of B3GNT2.

In an embodiment, the one or more agents are small molecules that bind the active site of B3GNT2 or comprise an allosteric inhibitor of B3GNT2.

In an embodiment, the one or more reagents is a programmable nuclease capable of reducing the expression of B3GNT2. In an embodiment, the one or more agents is a polynucleotide capable of inducing RNAi knockdown of B3GNT2 expression.

In certain example embodiments, are disclosed methods of enhancing anti-tumor immunity in a subject in need thereof comprising administering to the subject one or more agents that inhibit poly-N-acetyllactosamine (poly-LacNac) synthesis in tumor cells or reduces poly-LacNac on surface N-glycans. In an embodiment, the one or more agents are small molecule inhibitors of poly-LacNac synthesis selected from the group consisting of benzyl-O—N-acetylgalactosamide (BAG), kifunensine (KIF), tunicamycin, 3′-Azidothymidine (AZT), 2-acetamido-1,3,6-tri-O-acetyl-4-deoxy-4-fluoro-D-glucopyranose (4-F-GlcNac), and deoxymannojirimycin (DMN), whereby poly-LacNac synthesis is inhibited. In an embodiment, the one or more agents comprise an antibody that binds to a tumor-specific surface marker and is linked to an enzyme capable of cleaving poly-LacNac. In an embodiment, the enzyme is selected from the group consisting of endo H, endo F₂, endo F₃, peptide:N-glycosidase F (PNGase F), endo D, O-glycosidase, endo-b-galactosidase, sialidase and O-sialoglycoprotease.

In certain example embodiments, are disclosed methods of enhancing anti-tumor immunity in a subject in need thereof comprising administering to the subject one or more agents capable of inhibiting the expression or activity of MCL1. In an embodiment, the one or more agents increase the expression of one or more genes selected from the group consisting of BID, PMAIP1 (NOXA), BAX, BAK, BIM, BAD and PUMA. In an embodiment, the one or more agents is a small molecule selected from the group consisting of S63845, MIK665/S64315, AT101 (R-(−)-gossypol), TW-37, Gambogic acid, Sabutoclax (BI-97C1), Marinopyrrole A (maritoclax), UMI-77, A-1210477, Fesik's compounds, AMG176, AZD5991, Flavopiridol, Roscovitine, CR8, Voruciclib (P1446A-05), Cardiac glycoside, UNBS1450, Benzyl isothiocyanate, BAY43-9006, BAY1251152, BEZ235, AZD4573, AZD8055, SNS-032, dinaciclib, Arsenic trioxide Bufalin, and analogues thereof.

In an embodiment, the one or more agents is a programmable nuclease capable of reducing MCL1 activity. In an embodiment, the one or more agents is a polynucleotide capable of inducing RNAi knock down of MCL1 expression.

In certain example embodiments, is disclosed a method of enhancing anti-tumor immunity in a subject in need thereof comprising administering to the subject one or more agents capable of inhibiting the expression or activity of BCL2A1. In an embodiment, the one or more agents increase the expression of one or more genes selected from the group consisting of BID, PMAIP1 (NOXA), BAX, BAK, BIM, BAD and PUMA. In an embodiment, the one or more agents is a small molecule selected from the group consisting of AT101 (R-(−)-gossypol), TW-37, Gambogic acid, Sabutoclax (BI-97C1) and Marinopyrrole A (maritoclax). In an embodiment, the one or more agents is a programmable nuclease capable of reducing BCL2A1 expression or activity. In an embodiment, the one or more agents is a polynucleotide capable of inducing RNAi knock down of BCL2A1 expression.

In certain example embodiments, is disclosed a method of enhancing anti-tumor immunity in a subject in need thereof comprising administering to the subject one or more agents capable of inhibiting the expression or activity of JUNB. In an embodiment, the one or more agents is a dominant-negative JUNB protein or vector encoding the same. In an embodiment, wherein the one or more agents is a small molecule selected from the group consisting of curcumin, SP100030, SPC-839, T-5224, K1115A, momordin I, isosteviol and analogues thereof. In an embodiment, the one or more agents is a programmable nuclease capable of reducing JUNB expression or activity. In an embodiment, the one or more agents is a polynucleotide capable of inducing RNAi knock down of JUNB expression. In an embodiment, the one or more agents is capable of inhibiting the expression or activity of one or more downstream targets of JUNB selected from Table 5 or Table 6.

In certain example embodiments, the method disclosed herein further comprises administering an immunotherapy in combination with the one or more agents described above.

In an embodiment, the immunotherapy comprises adoptive cell transfer. In an embodiment, adoptive cell transfer comprises the administration of CAR (chimeric antigen receptor) T cells or natural killer cells, T cells expressing a T cell receptor (TCR) specific for a tumor antigen, or tumor infiltrating lymphocytes (TILs).

In an embodiment, the immunotherapy comprises checkpoint blockade (CPB) therapy. In an embodiment, the checkpoint blockade therapy comprises anti-CTLA4, anti-PD-L1, anti-PD1, anti-TIM3, anti-TIGIT, anti-LAG3, or combinations thereof.

In an embodiment, the subject is treated with an immunotherapy followed by the one or more agents described above. In an embodiment, the subject is treated with an immunotherapy followed by the one or more agents and an immunotherapy.

In an embodiment, the programmable nuclease is selected from the group consisting of a CRISPR system, a zinc finger nuclease, a TALE, or a meganuclease.

In certain example embodiments, is disclosed a method of monitoring the efficacy of an immunotherapy comprising detecting the expression of one or more genes selected from the group consisting of MCL1, BCL2A1, JUNB and B3GNT2 in a subject treated with an immunotherapy, wherein the subject is a non-responder to the immunotherapy if the one or more genes are upregulated after being treated. In an embodiment, the expression is detected at two or more time points during treatment, wherein a trend of increasing expression of the one or more genes indicates a poor outcome and/or a non-responder. In an embodiment, disclosed herein is a method of administering a treatment as described above if a poor outcome and/or a non-responder is indicated.

In certain example embodiments, is disclosed a method of treating a cancer in a subject in need thereof comprising determining if the patient is an immunotherapy responder or non-responder by detecting in a tumor obtained from the subject the expression or activity of one or more genes selected from candidate genes in Table 2, wherein if the expression of the one or more genes is higher than a reference value the subject is an immunotherapy non-responder and if the one or more genes is lower than a reference value then the subject is an immunotherapy responder; and if the subject is an immunotherapy non-responder, treating the subject using the method of any embodiment for enhancing an anti-tumor immune response herein; and if the subject is an immunotherapy responder, treating the subject with an immunotherapy.

In certain embodiments, is disclosed a method wherein the detecting comprises evaluating the expression or activity of 500 to 575 genes, of 400 to 500 genes, of 300 to 400 genes, of 200 to 300 genes, of 100 to 200 genes, of 50 to 100 genes, of 25 to 50 genes, of 10 to 25 genes, of 5 to 10 genes or of 1 to 5 genes selected from candidate genes in Table 2, wherein if the expression of the one or more genes is higher than a reference value the subject is an immunotherapy non-responder and if the one or more genes is lower than a reference value then the subject is an immunotherapy responder; and if the subject is an immunotherapy non-responder, treating the subject using the method of any embodiment for enhancing an anti-tumor immune response herein; and if the subject is an immunotherapy responder, treating the subject with an immunotherapy.

In certain embodiments, is disclosed a method wherein the detecting comprises evaluating the expression or activity of 1 to 5 genes selected from candidate genes in Table 2, wherein if the expression of the genes is higher than a reference value the subject is an immunotherapy non-responder and if the 1 to 5 genes is lower than a reference value then the subject is an immunotherapy responder; and if the subject is an immunotherapy non-responder, treating the subject using the method of any embodiment for enhancing an anti-tumor immune response herein; and if the subject is an immunotherapy responder, treating the subject with an immunotherapy. In certain embodiments, the one or more genes comprise B3GNT2, MCL1, BCL2A, JUNB, or a combination thereof.

In certain example embodiments, is disclosed a method of screening for agents capable of decreasing poly-LacNAc on tumor cells comprising a) contacting a population of tumor cells having upregulated B3GNT2 with an agent and b) detecting binding of one or more proteins to the tumor cells selected from the group consisting of CD2, 4-1BB, TREML2 (TLT2), NKG2D, and an antibody specific for an HLA class I bound tumor antigen, wherein increased binding indicates reduced poly-LacNAc. In an embodiment, the one or more proteins are labeled with a detectable marker.

In an example embodiment, is disclosed a method of enhancing anti-tumor immunity in a subject in need thereof comprising administering to the subject one or more agents capable of inhibiting the expression or activity of one or more targets selected from Table 1 or Table 3.

In certain example embodiments, is disclosed a T cell that expresses an enzyme capable of cleaving poly-LacNac on the T cell surface.

In an embodiment, the enzyme is selected from the group consisting of endo H, endo F₂, endo F₃, peptide:N-glycosidase F (PNGase F), endo D, O-glycosidase, endo-β-galactosidase, sialidase and O-sialoglycoprotease. In an embodiment, a method is disclosed of enhancing anti-tumor immunity in a subject in need thereof comprising administering to the subject the T cell that expresses an enzyme capable of cleaving poly-LacNac on the T cell surface. These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 (A-K)— Establishing a genome-scale CRISPR activation screen for resistance to T cell cytotoxicity. (FIG. 1A) Percent indels generated by different CRISPR knockout (KO) sgRNAs targeting CTAG1A/B genes that encode for the NY-ESO-1 antigen. N=3. (FIG. 1B-C) Cell survival of NY-ESO-1+ and HLA-A2 A375 melanoma cells with different (TAG1A/B KO sgRNAs that were co-cultured with T cells expressing the NY-ESO-1 T cell receptor (ESO T cells) relative to cells that have not been exposed to T cells (B) or cells co-cultured with unmodified T cells (C). N=8. NT, non-targeting. Percent survival at different effector to target (E:T) ratios were measured. (FIG. 1D-K) Results of the acute and chronic exposure screens showing normalized sgRNA counts as counts per million (CPM; D-E), sgRNA enrichment in the ESO T cell condition relative to control (F-G), gene enrichment determined by the average sgRNA enrichment (H-I), and MAGeCK analysis P-values (J-K). The two most enriched genes from each screening strategy are highlighted in red. All values are mean±s.e.m. ****P<0.0001; ***P<0.001; *P<0.05; ns, not significant.

FIG. 2 (A-F)—Genome-scale CRISPR activation screen identifies four candidate genes that confer resistance to T cell cytotoxicity. (FIG. 2A) Schematic of the CRISPRa screen. NY-ESO-1⁺ and HLA-A2⁺ A375 melanoma cells were transduced with the pooled sgRNA library targeting more than 23,000 coding isoforms. A375 cells were exposed to primary CD4⁺ and CD8⁺ T cells expressing the T cell receptor (TCR) specific for the NY-ESO-1 antigen. Deep sequencing identified candidate genes. (FIG. 2B) Average MAGeCK analysis P-values for the acute and chronic exposure screens. Top candidate genes are annotated and the two most enriched genes from each screening strategy are highlighted in red. (FIG. 2C) Most significant pathways enriched among the 576 candidate genes. (FIG. 2D) Heatmap showing Pearson's correlation between expression of the top four candidate genes and cytolytic activity across patient tumors from TCGA. Only significant (FDR<0.05) correlations are shown. (FIG. 2E) Box plots showing single-sample Gene Set Enrichment Analysis (ssGSEA) (Barbie et al., 2009) of 576 candidate genes in 310 patient tumor samples (Auslander et al., 2018; Braun et al., 2020; Gide et al., 2019; Hugo et al., 2016; Pender et al., 2021; Riaz et al., 2017). (FIG. 2F) Cell survival of A375 cells transduced with ORFs encoding candidate genes against ESO T cell cytotoxicity at different effector to target (E:T) ratios. Cell survival was measured using a luminescent cell viability assay and normalized to paired control cells that were not cultured with T cells. T cells were derived from donors used in the CRISPRa screen. All values are mean±s.e.m. with n=12. ****P<0.0001.

FIG. 3 (A-H)—Validation of four candidate genes for resistance to T cell cytotoxicity. (FIG. 3A) Heatmap showing Pearson's correlation between cytolytic activity and expression of the top 576 candidate genes across patient tumors from TCGA. Only significant (FDR<0.05) correlations are shown. (FIG. 3B) Number of candidate genes with significant (FDR<0.05) positive or negative Pearson's correlation across patient tumors from TCGA. (FIG. 3C-D) Heatmaps showing significant Pearson's correlations between cytolytic activity and expression of 291 negative control housekeeping genes (FIG. 3C), or four positive control genes known to promote resistance upon overexpression (FIG. 3D) across patient tumors from TCGA. (FIG. 3E-F) Average MAGeCK analysis P-values of genes that promote A375 cell growth (FIG. 3E) or death (FIG. 3F) determined by comparing the distribution of sgRNAs in the CRISPRa screen control conditions to the initial sgRNA library. (FIG. 3G) Enrichment of CRISPR activation sgRNAs targeting each candidate gene for different screening biological replicates (bioreps). (FIG. 3H) T cell cytotoxicity resistance (n=12) and transcriptional upregulation (n=4) in A375 cells upon CRISPR activation of candidate genes. NT, non-targeting. All values are mean±s.e.m. ****P<0.0001; ***P<0.001; **P<0.01; ns, not significant.

FIG. 4 (A-E)—Relevance of top four candidate genes in patient tumors. (FIG. 4A) Box plots showing expression of candidate genes in TCGA datasets for 31 types of human cancers. Genes that were significantly differentially expressed between tumor and matched with normal tissues are indicated. (FIG. 4B) Copy number variation of candidate genes represented as percent of cases for each type of cancer in TCGA. (FIG. 4C) Comparison of candidate gene expression and clinical response to anti-PD-1 treatment using patient data from the Hugo et al. dataset (Hugo et al., 2016). Mean±s.e.m. is shown. (FIG. 4D) Comparison of candidate gene expression before (pre) and after (post) anti-PD-1 treatment and clinical response using patient data from the Riaz et al. dataset (Riaz et al., 2017). *P<0.05. (FIG. 4E) Pearson's correlation between candidate gene expression and cytolytic score (Rooney et al., 2015) prior to treatment using patient data from Riaz et al. (Riaz et al., 2017).

FIG. 5 (A-E) Candidate gene overexpression mediates resistance in other cell types and in vivo. (FIG. 5A) Cell survival against ESO T cell cytotoxicity of H1793 (NY-ESO-1⁺, HLA-A2⁻) non-small cell lung adenocarcinoma transduced with HLA-A2 and ORFs encoding candidate genes. N=12. (FIG. 5B) Heatmap summarizing results from ESO T cell cytotoxicity assays for 8 cell lines derived from different tissues. Each value represents significance of the difference between survival of each ORF and GFP control. (FIG. 5C) Schematic of the in vivo experiments to test the response of A375 xenografts overexpressing candidate genes to adoptive cell transfer (ACT) in NSG mice. (FIG. 5D-E) Tumor growth in mice receiving ACT of ESO T cells. Tumor volume (FIG. 5D) and overall host survival (FIG. 5E) are shown. Data is representative of two independent experiments. N=12. All values are mean±s.e.m. ****P<0.0001; ***P<0.001; **P<0.01

FIG. 6 (A-H)—Candidate genes mediate resistance to T cell cytotoxicity across different co-culture conditions. (FIG. 6A) Cell survival against ESO T cell-mediated cytotoxicity in A375 cells overexpressing candidate gene ORFs. T cells were derived from donors that were not used in the CRISPRa screen. n=8. (FIG. 6B) Cell survival against ESO T cell-mediated cytotoxicity in A375 cells overexpressing candidate gene ORFs over time. Viability was measured using an alternative assay based on secreted Gaussia luciferase. n=8. (FIG. 6C) Growth of A375 cells overexpressing candidate gene ORFs in the absence of ESO T cell co-culture over time measured using secreted Gaussia luciferase. Data was fitted using the Malthusian exponential growth Y(t)=Yoekt, with 1=time, Y=luminescence, k=growth rate. The growth rates were compared using the extra sum-of-squares F test. n=8. (FIG. 6D) Growth of A375 cells overexpressing candidate gene ORFs in the absence of ESO T cell co-culture measured using a luminescent cell viability assay from 5 independent experiments with n=44. (FIG. 6E) Dox-induction of candidate genes in A375 cells. Cell survival against ESO T cell cytotoxicity (n=8) and candidate gene expression relative to ACTB control (n=4) were measured at different Dox concentrations. (FIG. 6F) Expression of candidate genes in 1,019 cell lines from the Cancer Cell Line Encyclopedia (Barretina et al., 2012). Expression in A375 cells and the expression threshold above which the candidate gene conferred resistance to T cell cytotoxicity in A375 cells are indicated. The percentage of cell lines with gene expression above the resistance threshold is shown. (FIG. 6G) Cell survival of A375 cells overexpressing candidate genes against CD4⁺, CD8⁺, or CD4⁺ and CD8⁺ T cell cytotoxicity. n=8. (FIG. 6H) Cell survival of A375 cells overexpressing candidate genes against T cells expressing the AXL chimeric antigen receptor (CAR). n=8. All values are mean±s.e.m. ****P<0.0001; ***P<0.001; **P<0.01; *P<0.05; ns, not significant.

FIG. 7 (A-J)—Candidate genes mediate resistance to T cell cytotoxicity in other cell types and in vivo. (FIG. 7A) Expression level of the NY-ESO-1 antigen represented as the average expression of CTAG1A and CTAG1B genes. RNA-seq expression data for each cell line was obtained from the Cancer Cell Line Encyclopedia (Barretina et al., 2012). (FIG. 7B-G) Resistance to ESO T cell-mediated cytotoxicity in SW1417 (NY-ESO-1⁻, HLA-A2⁻) colorectal adenocarcinoma (FIG. 7B), OAW28 (NY-ESO-1⁺, HLA-A2⁻) ovarian cystadenocarcinoma (C), H1299 (NY-ESO-1⁺, HLA-A2⁻) non-small cell lung carcinoma (FIG. 7D), A2058 (NY-ESO-1⁻, HLA-A2⁻) melanoma (FIG. 7E), LN-18 (NY-ESO-1⁺, HLA-A2⁺) glioblastoma (FIG. 7F), or SK-N-AS (NY-ESO-1⁺, HLA-A2⁻) neuroblastoma (FIG. 7G) cell lines. Cell lines that did not endogenously express HLA-A2 or NY-ESO-1 were transduced with the respective constructs. n=8 (B, C, E) or 12 (D, F, G). (FIG. 7H) Heatmap showing average expression (n=4) relative to ACTB of candidate genes in various cell lines overexpressing the ORF or GFP. (FIG. 7I-J) Tumor growth in control mice that did not receive adoptive cell transfer (ACT). Tumor volume (FIG. 7I) and overall survival (FIG. 7J) are shown. n=6. All values are mean±s.e.m. ****P<0.0001; ***P<0.001; **P<0.01; *P<0.05; ns, not significant.

FIG. 8 (A-D)—Effect of candidate gene overexpression on the transcriptome and IFNγ pathways. (FIG. 8A) Volcano plots showing transcriptome changes measured by RNA-seq in A375 cells overexpressing candidate genes. The number of genes that were significantly differentially expressed with P-value≥0.01 FDR correction are indicated. Out of these genes, those with |log₂(fold change)|≥1 are shown as red dots and the number of genes is indicated in parentheses. n=3. (FIG. 8B) T cell cytotoxicity resistance (n=8) and transcriptional upregulation (n=4) in A375 cells overexpressing N- or C-terminal FLAG tagged (N-FLAG or C-FLAG respectively) JUNB or B3GNT2. (FIG. 8C) IFNγ measured by ELISA in cell culture media of ESO T cells co-cultured with A375 cells overexpressing candidate genes. n=16. (FIG. 8D) Western blots of phosphorylated or total STAT1 in A375 cells overexpressing candidate genes that have been exposed to different concentrations of IFNγ. All values are mean±s.e.m. ****P<0.0001; ***P<0.001; **P<0.01; *P<0.05; ns, not significant.

FIG. 9 (A-G)—MCL1 and JUNB mediate resistance to FasL- and TRAIL-induced cell death through the mitochondrial apoptosis pathway. (FIG. 9A) Caspase 8 activity measured using a colorimetric cleavage assay in A375 cells overexpressing candidate genes after treating with 500 ng/μL of FasL or TRAIL for 3 hours. N=3. (FIG. 9B) Dox-induction of genes in the mitochondrial apoptosis pathway in A375 cells overexpressing MCL1 or GFP. Cell survival against ESO T cell cytotoxicity (n=8) and expression of MCL1 interaction partners (n=4) were measured at different Dox concentrations. (FIG. 9C) Histograms showing expression of cell surface Fas or TRAIL receptor, FAS or TNFRSF10B, measured by antibody staining and flow cytometry in A375 cells overexpressing candidate genes. N=3. (FIG. 9D) Cell survival against 500 ng/μL of FasL- or TRAIL-induced cell death in A375 cells overexpressing JUNB or GFP with BCL2A1 knocked down. N=4. KD, knockdown. NT, non-targeting. (FIG. 9E) RNA-seq expression of NF-κB pathway genes overlapping JUNB ChIP-seq measured as fold change in A375 cells overexpressing JUNB relative to GFP control. N=3. (FIG. 9F) Western blots of phosphorylated or total p65 (RELA) protein in A375 cells overexpressing candidate genes. (FIG. 9G) Schematic describing the pathways MCL1 and JUNB are involved in to mediate resistance to T cell cytotoxicity. All values are mean±s.e.m. ****P<0.0001; ***P<0.001; **P<0.01; *P<0.05; ns, not significant.

FIG. 10 (A-F)—MCL1 and JUNB mediate survival by resisting FasL- and TRAIL-induced cytotoxicity. (FIG. 10A) Cell survival of A375 cells overexpressing candidate genes against different concentrations of FasL, TRAIL, or TNFα. N=4. (FIG. 10B) Western blots showing Caspase 8 cleavage products in A375 cells overexpressing candidate genes after treating with 500 ng/μL of FasL or TRAIL for 3 hours. (FIG. 10C) Dox-induction of different RefSeq isoforms of genes in the mitochondrial apoptosis pathway in A375 cells overexpressing MCL1. Cell survival against ESO T cell-induced cytotoxicity (n=8) and expression (n=4) of MCL1 interaction partners were measured at different Dox concentrations. (FIG. 10D) Expression of BCL2A1 in A375 cells overexpressing JUNB or B3GNT2 with BCL2A1 knocked down. N=4. KD, knockdown. NT, non-targeting. (FIG. 10E) Cell survival against 125 ng/μL of FasL- or TRAIL-induced cell death in A375 cells overexpressing JUNB or GFP with BCL2A1 knocked down. N=4. (FIG. 10F) Cell survival against ESO T cells in A375 cells overexpressing JUNB or GFP with BCL2A1 knocked down. N=8. All values are mean±s.e.m. ****P<0.0001; ***P<0.001; **P<0.01; *P<0.05; ns, not significant.

FIGS. 11 (A-F)—B3GNT2 adds poly-LacNAc to tumor ligands and receptors to promote tumor immune evasion. (FIG. 11A) Intra- and extra-cellular poly-LacNAc measured by tomato lectin staining in A375 cells overexpressing B3GNT2 or GFP that were treated with different concentrations of kifunensine or benzyl-2-acetamido-2-deoxy-α-D-galactopyranoside (BAG) glycosylation inhibitors. N=3. (FIG. 11B) Cell survival against T cell cytotoxicity (top) and T cell IFNγ secretion (bottom) in A375 cells overexpressing B3GNT2 or GFP that have been treated with different concentrations of BAG at E:T ratio of 3. N=6. (FIG. 11C-D) Western blots of different tumor ligands and receptors that interact with T cells in A375 cells overexpressing candidate genes. For a subset of the ligands and receptors that were potentially glycosylated (D), enzymatic deglycosylation was performed to confirm presence of N- or O-glycosylation. (FIG. 11E) Western blots of tumor cell surface ligands and receptors that interact with T cells in SW1417 colorectal adenocarcinoma cells. Cells were treated with kifunensine (KIF) or BAG to remove N- or O-glycosylation respectively. (FIG. 11F) Co-IP of N- or C-terminal FLAG tagged (N-FLAG or C-FLAG) B3GNT2 followed by Western blot for different B3GNT2 target proteins. 2% of the input and IgY IP controls are shown. All values are mean±s.e.m. ****P<0.0001; **P<0.01; *P<0.05; ns, not significant.

FIGS. 12 (A-D)—B3GNT2 disrupts ligand-receptor interactions between tumor and T cells. (FIG. 12A) Cell survival against T cell cytotoxicity (top) and T cell IFNγ secretion (bottom) in A375 cells overexpressing B3GNT2 or GFP that have been treated with different concentrations of kifunensine at E:T ratio of 3. N=6. (FIG. 12B) Western blots of tumor cell surface ligands and receptors that interact with T cells in A375 cells overexpressing B3GNT2 or GFP. Cells were treated with kifunensine (KIF) or benzyl 2-acetamido-2-deoxy-α-D-galactopyranoside (BAG) to remove N- or O-glycosylation respectively. (FIG. 12C) Histograms showing binding of recombinant T cell proteins to A375 cells measured by flow cytometry. A375 cells were overexpressing GFP or B3GNT2 and treated with KIF or BAG. N=3. (FIG. 12D) Schematic showing the tumor cell surface ligands and receptors modified by B3GNT2 to disrupt interactions with T cells that mediate cytotoxicity. All values are mean±s.e.m. ****P<0.0001; ***P<0.001; **P<0.01; *P<0.05; ns, not significant.

FIG. 13 (A-J)—Inhibition of candidate genes increases susceptibility of tumors to T cell killing upon knockdown. (FIG. 13A) Heatmap showing expression of candidate genes in different cell lines transduced with CRISPR knockdown (KD) sgRNAs relative to non-targeting (NT) sgRNAs. N=4. (FIG. 13B) Percent indels generated by different CRISPR knockout (KO) sgRNAs targeting candidate genes in A375 melanoma and SW1417 colorectal adenocarcinoma cells. N=3. (FIG. 13C-H) Cell survival against T cell-mediated cytotoxicity with different candidate genes knocked down (C-E) or knocked out (F-H). Cell lines that did not endogenously express HLA-A2 or NY-ESO-1 were transduced with the respective constructs. N=8. c, SW1417 (NY-ESO-1⁻, HLA-A2⁻) colorectal adenocarcinoma against ESO T cells. (FIG. 13D) A375 (NY-ESO-1⁺, HLA-A2⁺) melanoma against ESO T cells. (FIG. 13E) OAW28 (NY-ESO-1⁺, HLA-A2⁻) ovarian cystadenocarcinoma against ESO T cells. (FIG. 13F) SW1417 (HER2⁺) colorectal adenocarcinoma against HER2 CAR T cells. (FIG. 13G) SW1417 (NY-ESO-1⁻, HLA-A2⁻) colorectal adenocarcinoma against ESO T cells. (FIG. 13H) A375 (NY-ESO-1⁺, HLA-A2⁺) melanoma against ESO T cells. (FIG. 13I-J) Cell survival against T cell cytotoxicity in tumor cells treated with small molecule inhibitors targeting MCL1 (S63845 and AZD5991) or B3GNT2 (kifunensine). N=8. (FIG. 13I) A375 (NY-ESO-1⁺, HLA-A2⁺) melanoma against ESO T cells. (FIG. 13J) CCLF_MELM_0011 T (HER2⁺) primary patient-derived melanoma model against HER2 CAR T cells. All values are mean±s.e.m. ****P<0.0001; ***P<0.001; **P<0.01; *P<0.05; ns, not significant.

FIGS. 14 (A-H)—B3GNT2 overexpression disrupts binding of T cell ligands and receptors to tumor cells. (FIG. 14A) Histograms showing binding of various T cell ligands or receptor proteins to A375 cells overexpressing candidate genes measured by flow cytometry. (FIG. 14B) Histogram showing binding of an antibody specific for HLA-A2:NY-ESO-1 to A375 cells overexpressing B3GNT2 or GFP that have been treated with kifunensine (KIF). (FIG. 14C) Percent indels generated by different CRISPR knockout (KO) sgRNAs targeting 4-1BBL (TNFSF9) in A375 cells. (FIG. 14D) Protein expression of 4-1BBL after CRISPR KO. (FIG. 14E) Histogram showing binding of 4-1BB to A375 cells with different KO sgRNAs targeting 4-1BBL. (FIG. 14F) CD276 expression in A375 cells with different CRISPR knockdown (KD) sgRNAs. (G) Protein expression of CD276 after CRISPR KD. (FIG. 14H) Histogram showing binding of TREML2 to A375 cells with different KD sgRNAs targeting CD276. All values are mean±s.e.m. with n=3. NT, non-targeting; ****P<0.0001; ***P<0.001; **P<0.01; *P<0.05; ns, not significant.

FIG. 15 (A-E)—Inhibition of candidate genes sensitizes tumors to T cell cytotoxicity. (FIG. 15A) Cell survival against HER2 CAR T cell cytotoxicity in SW1417 (HER2⁺) colorectal adenocarcinoma with different candidate genes knocked down using CRISPRi and 2 sgRNAs per gene. KD, knockdown. NT, non-targeting. (FIG. 15B-E) Cell survival against T cell cytotoxicity in tumor cells treated with MCL1 or B3GNT2 small molecule inhibitors, S63845 and kifunensine respectively. (FIG. 15B) A375 (NY-ESO-1⁺, HLA-A2*) melanoma against ESO T cells. (FIG. 15C) SW1417 (HER2⁺) colorectal adenocarcinoma against HER2 CAR T cells. (FIG. 15D) CCLF_MELM_0011_T (AXL⁺) primary patient-derived melanoma model against AXL CAR T cells. (FIG. 15E) CCLF_PANC_0014_T (HER2⁺) primary patient-derived pancreatic adenocarcinoma against HER2 CAR T cells. All values are mean±s.e.m with n=8. ****P<0.0001; ***P<0.001; **P<0.01; *P<0.05; ns, not significant.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4^{th} ed., John Wiley & Sons (New York, N. Y. 1992); and Marten H. Hofker and Jan van Deursen, Transgenic Mouse Methods and Protocols, 2nd edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide novel gene targets, B3GNT2, MCL1, BCL2A1 and JUNB, which when overexpressed, enabled tumor cells to evade killing by cytotoxic T cells. These four gene targets have not heretofore been associated with immune evasion or to confer resistance to diverse cancer cell types and mouse xenografts. The importance of immune evasion by tumors is exemplified by the introduction and success of T cell targeted immunomodulators blocking the immune checkpoints CTLA-4 and PD1 or PDL1 (immune checkpoint inhibitors (ICI)). Anti-PD1/PDL1 antibodies have become some of the most widely prescribed anti-cancer therapies. T-cell-targeted immunomodulators are now used as single agents or in combination with chemotherapies as first or second lines of treatment for about 50 cancer types. Thus, immunotherapy leads the field in cancer research owing to the clinical success of ICI and the need to identify additional immune evasion targets is greater than ever.

In one aspect, embodiments disclosed herein are directed to methods of enhancing anti-tumor immunity by administering one or more agents that reduce the expression or activity of B3GNT2. In another aspect, embodiments disclosed herein are directed to methods of enhancing anti-tumor immunity by administering one or more agents that inhibit poly-N-acetyl-lactosamine (poly-LacNAc) synthesis in tumor cells, or by reducing poly-LacNac on surface N-glycans. In another aspect, embodiments disclosed herein are directed to methods of enhancing anti-tumor immunity by administering one or more agents capable of inhibiting the expression or activity of MCL1. In another aspect, embodiments disclosed herein are directed to methods of enhancing anti-tumor immunity by administering one or more agents that reduce the expression or activity of BCL2A1. In another aspect, embodiments disclosed herein are directed to methods of enhancing anti-tumor immunity by administering one or more agents that reduce the expression or activity of JUNB.

In another aspect, the therapeutic methods disclosed above may be administered in combination with an immunotherapy. In one example embodiment, the immunotherapy may comprise adoptive cell transfer of immune cells specific for a tumor. In one example embodiment, the cell transfer comprises the administration of CAR (chimeric antigen receptor) T cells or natural killer cells, T cells expressing a T cell receptor (TCR) specific for a tumor antigen, or tumor infiltrating lymphocytes (TILs). In one example embodiment, the immunotherapy comprises checkpoint blockade (CPB) therapy. In one example embodiment, the checkpoint blockade therapy comprises anti-CTLA4, anti-PD-L1, anti-PD1, anti-TIM3, anti-TIGIT, anti-LAG3, or combinations thereof.

In another aspect, embodiments disclosed herein are directed to methods of monitoring the efficacy of an immunotherapy comprising detecting the expression of one or more genes selected from the group consisting of B3GNT2, MCL1, BCL2A1, and JUNB in a subject treated with an immunotherapy, wherein the subject is a non-responder to the immunotherapy if the one or more genes are upregulated after being treated.

In another aspect, embodiments disclosed herein are directed to methods of screening for agents capable of decreasing poly-LacNac on tumor cells comprising a) contacting a population of tumor cells having upregulated B3GNT2 with an agent; and b) detecting binding of one or more proteins to the tumor cells selected from the group consisting of CD2, 4-1BB, TREML2 (TLT2), NKG2D, and an antibody specific for an HLA class I bound tumor antigen, wherein increased binding indicates reduced poly-LacNac.

In another aspect, embodiments disclosed herein are directed to methods of enhancing anti-tumor immunity in a subject in need thereof comprising administering to the subject one or more agents capable of inhibiting the expression or activity of one or more targets selected from Table 1, Table 2 (candidate genes) or Table 3.

### Method of Enhancing Anti-Tumor Immunity Through Inhibition of B3GNT2 Protein Activity

The β1,3-N-acetylglucosaminyltransferase 2 (B3GNT2) enzyme is encoded by the B3GNT2 gene and is involved in poly-N-acetyl-lactosamine (poly-LacNac) synthesis). As with other β1,3-N-acetylglucosaminyltransferases, the enzyme is a Golgi-resident glycosyltransferase. Poly-N-acetyl-lactosamine (poly-LacNAc) structures are composed of repeating [-Galβ(1,4)-GlcNAcβ(1,3)-]n glycan extensions. Further, they are found on both N- and O-glycoproteins and glycolipids and play an important role in development, immune function, and human disease. The majority of mammalian poly-LacNAc is synthesized by the alternating iterative action of B3GNT2 and β1,4-galactosyltransferases. B3GNT2 is in the largest mammalian glycosyltransferase family, GT31. As discussed further herein, the Applicant has identified B3GNT2 expression as up-regulated in tumors that have become non-responsive to immunotherapy, including use of checkpoint inhibitors and adoptive cell transfer. Additionally, reducing B3GNT2 activity shifts tumors from an immunotherapy non-responsive to a responsive phenotype.

In one example embodiment, a method of enhancing anti-tumor immunity comprises administering one or more agents capable of inhibiting the expression or activity of B3GNT2. In one example embodiment, the method comprises administering one or more small molecules that inhibit B3GNT2 activity. In another example embodiment, the method comprises administering a RNAi polynucleotide to knockdown expression of B3GNT2. In another example embodiment, the method comprises delivering a programmable nuclease to reduce expression of B3GNT2.

### Small Molecule Inhibitors of B3GNT2

In one example embodiment, a method of enhancing anti-tumor immunity in a subject comprises administering one or more small molecules that inhibit B3GNT2 activity. The small molecule inhibits may bind to B3GNT2's active site or function as an allosteric inhibitor of B3GNT2 activity.

In one example embodiment, B3GNT2 protein activity is inhibited by administering one or more small molecule agents selected from the group consisting of benzyl-O—N-acetylgalactosamide (BAG), kifunensine (KIF), tunicamycin, 3′-Azidothymidine (AZT), 2-acetamido-1,3,6-tri-O-acetyl-4-deoxy-4-fluoro-D-glucopyranose [4-F-GlcNAc], and deoxymannojirimycin (DMN).

### Method of Enhancing Anti-Tumor Immunity Through Inhibition of MCL1 Protein Function

A tumor may block an apoptosis pathway in order to evade killing by an anti-tumor immune response. In certain embodiments, a tumor overexpresses MCL1. Apoptosis is a crucial process by which multicellular organisms control tissue growth, removal and inflammation. Disruption of the normal apoptotic function is often observed in cancer, where cell death is avoided by the overexpression of anti-apoptotic proteins of the Bcl-2 (B-cell lymphoma) family, including MCL1 (myeloid cell leukemia). This makes MCL1 a potential target for drug therapy, through which normal apoptosis may be restored by inhibiting the protective function of MCL1.

Myeloid cell leukemia-1 (MCL1) is an anti-apoptotic member of the B-cell lymphoma-2 (BCL-2) family of proteins that regulates apoptosis. Alternative splicing occurs for MCL1 and two transcript variants encoding distinct isoforms have been identified. The longer gene product (isoform 1, MCL-1L) enhances cell survival by inhibiting apoptosis while the alternatively spliced shorter gene product (isoform 2 MCL-1S) promotes apoptosis and is death-inducing. In certain embodiments, isoform 1 is targeted. The members of the Bel-2 family are designated as such due to their BCL-2 homology (BH) domains and involvement in apoptosis regulation. The BH domains facilitate the family members' interactions with each other, and can indicate pro- or anti-apoptotic function. Traditionally, these proteins are categorized into one of three subfamilies; anti-apoptotic, BH3-only (pro-apoptotic), and pore-forming or ‘executioner’ (pro-apoptotic) proteins (BAX and BAK) (see, e.g., Warren C F A, Wong-Brown M W, Bowden N A. BCL-2 family isoforms in apoptosis and cancer Cell Death Dis. 2019; 10(3): 177; and Vogler, M. BCL2A1: the underdog in the BCL2 family. Cell Death Differ 19, 67-74 (2012)). MCL-1 exerts its antiapoptotic function by sequestering proapoptotic proteins BAK/BAX through the BH3 domain containing hydrophobic groove. In the prosurvival mode, BAK/BAX interacts with antiapoptotic BCL-2 proteins and is unable to execute the apoptotic program, thereby allowing cells to maintain homeostasis (see, e.g., Xiang W, Yang C Y, Bai L. MCL-1 inhibition in cancer treatment. Onco Targets Ther. 2018; 11:7301-7314). BH3-only proteins, BIM, PUMA, BAD, NOXA and BID, restore BAX/BAK activities through interruption of the MCL-1:BAK/BAX complexes. Id.

Elevated levels of MCL1 contribute to tumorigenesis and resistance to conventional chemotherapies and targeted therapies, including the BCL-2 selective inhibitor venetoclax. Applicants discovered that MCL1 overexpression is used to evade an anti-tumor immune response and MCL1 inhibitors can be used to enhance an immunotherapy. Accordingly, in some embodiments disclosed herein, are methods designed to downregulate MCL1 expression or inhibit MCL1 for treating cancer, preferably in combination with an immunotherapy. In certain embodiments, subjects being treated with an immunotherapy have increased expression of MCL1 and become non-responsive. In certain embodiments, administering an agent targeting MCL1 expression or activity shifts a subject non-responsive to an immunotherapy to a responder. In certain embodiments, the one or more therapeutics described herein are administered to a subject that has a tumor overexpressing MCL1. The present invention also provides for determining subjects that may respond to an MCL1 inhibitor. For example, the tumor overexpresses MCL1 and does not overexpress another protein that allows evasion of an immune response.

Human cancers are genetically and epigenetically heterogeneous and have the capacity to commandeer a variety of cellular processes to aid in their survival, growth and resistance to therapy. One such strategy is to overexpress proteins that suppress apoptosis, such as the BCL-2 family protein MCL1. The MCL1 protein plays a pivotal role in protecting cells from apoptosis and is overexpressed in a variety of human cancers. In an embodiment, a method of enhancing anti-tumor immunity in a subject in need thereof comprises administering to the subject one or more agents capable of inhibiting the expression or activity of MCL1.

### Small Molecule Inhibitors of MCL1

Dysregulation of the mitochondrial apoptotic pathway controlled by members of the BCL-2 protein family plays a central role in cancer development and resistance to conventional cytotoxic as well as targeted therapies. The selective inhibition of the pro-survival BCL-2 family of proteins to activate apoptosis in malignant cells represents an anti-cancer strategy. The remarkable clinical performance of the selective BCL-2 antagonist Venetoclax has highlighted the potential for selective inhibitors of the other pro-survival members of the BCL-2 family, particularly MCL1. Venetoclax is a BH3-mimetic and can block the anti-apoptotic B-cell lymphoma-2 (Bcl-2) family proteins. The present invention also provides for use of the small molecules in combination with an immunotherapy treatment regimen. In certain embodiments, small molecules are administered in doses that do not induce apoptosis in the absence of an effective anti-tumor immune response. Thus, doses required to shift a subject from a nonresponder to a responder are lower than doses required to induce apoptosis and the doses of the present invention are lower than certain standard doses. In particular, doses are lower in a combination immunotherapy treatment regimen.

In certain embodiments disclosed herein, the methods provide several drugs or small molecules that cause downregulation of MCL1 expression or a decrease in activity as part of their mechanism of action. Pan-cyclin-dependent kinase inhibitors, such as flavopiridol, SNS-032, CYC202 (Roscovitine) and dinaciclib, or the selective CDK9 inhibitors BAY1251152 and AZD4573 have been reported to kill cells in part by blocking MCL1 transcription (Chen et al., 2009; Cidado et al., 2018; Fu et al., 2011; Gojo, Zhang et al. 2002; Luecking et al., 2017). Chemotherapeutic drugs such as anthracyclines were found to preferentially repress MCL1 transcription to induce apoptosis in tumor cells (Wei et al., 2012). These small molecules that target MCL1 expression or activity are hereby specifically incorporated by reference.

In certain example embodiments disclosed herein, the methods provide other drugs or small molecules capable of inhibiting the expression or activity of MCL1 and include S63845, MIK665/S64315, AT101 (R-(−)-gossypol), TW-37, Gambogic acid, Sabutoclax (BI-97C1), Marinopyrrole A (maritoclax), UMI-77, A-1210477, Fesik's compounds, AMG176, AZD5991, Flavopiridol, Roscovitine, CR8, Voruciclib (P1446A-05), Cardiac glycoside, UNBS1450, Benzyl isothiocyanate, BAY43-9006, BEZ235, AZD8055, BEZ235, AZD8055, and arsenic trioxide Bufalin (see, e.g., Xiang W, Yang C Y, Bai L. MCL-1 inhibition in cancer treatment. Onco Targets Ther. 2018; 11:7301-7314).

In a certain example embodiment, disclosed herein is provided a method for inhibiting MCL1 expression or activity by administering one or more small molecule agents to a subject in need thereof. In embodiments, the present disclosure provides one or more small molecule agents selected from the group consisting of S63845, MIK665/S64315, AT101 (R-(−)-gossypol), TW-37, Gambogic acid, Sabutoclax (BI-97C1), Marinopyrrole A (maritoclax), UMI-77, A-1210477, Fesik's compounds, AMG176, AZD5991, Flavopiridol, Roscovitine, CR8, Voruciclib (P1446A-05), Cardiac glycoside, UNBS1450, Benzyl isothiocyanate, BAY43-9006, BAY1251152, BEZ235, AZD4573, AZD8055, SNS-032, dinaciclib, BEZ235, AZD8055, arsenic trioxide Bufalin, and analogues thereof.

MCL1 protein is unique compared to its pro-survival BCL-2 relatives. MCL1 expression is regulated at the transcriptional level by trophic factors such as granulocyte-macrophage stimulating factor, epidermal growth factor and cytokines (Chao et al., 1998; Huang, et al., 2000; Jourdan et al, 2000). At the intracellular level, transcription of MCL1 is controlled by several signaling pathways, including the PI3K/AKT, p38/MAPK and STAT3 pathways (Akgul, 2009; Becker et al., 2014; Huelsemann et al., 2014). In addition, N10 miRNA has been identified to modulate MCL1 translation, including miR-29 and miR-125b, and are reviewed elsewhere (Cui et al, 2018).

In some embodiments, the methods of inhibiting or downregulating MCL1 expression or activity may further comprise administering the above trophic factors (e.g., EGF) and modulating the above signaling pathways. These trophic factors and signaling pathways that target MCL1 expression or activity are hereby specifically incorporated by reference.

Embodiments of the present disclosure take into consideration the pro-survival role MCL1 has in many normal tissues. MCL1 is ubiquitously expressed and essential for embryonic development as demonstrated by studies performed in mice with homozygous loss of MCL1 (Kozopas, et al, 1993; Rinkenberger et al., 2000). Conditional MCL1 knockout mice showed that MCL1 also plays a critical role in the survival of hematopoietic stem cells, lymphocytes and cardiomyocytes, among other normal cells (Opferman et al., 2003; Opferman et al., 2005; Thomas et al., 2013). However, it is possible that some of the observed effects in normal tissues are caused by chronic depletion of MCL1 or other activities besides their anti-apoptotic function and could potentially be mitigated by careful dosing and schedule of MCL1 inhibitors in disease treatment. Accordingly, embodiments of the present disclosure take into consideration the clinical applications of administering MCL1 inhibitors and the pro-survival role of MCL1.

### Method of Enhancing Anti-Tumor Immunity Through Inhibition of BCL2A1 Protein Activity

BCL2A1 encodes a member of the bcl2 protein family. The proteins of this family form hetero- or homodimers and act as anti- and pro-apoptotic regulators that are involved in a wide variety of cellular activities such as embryonic development, homeostasis and tumorigenesis. The protein encoded by this gene is able to reduce the release of pro-apoptotic cytochrome c from mitochondria and block caspase activation. This gene is a direct transcription target of NF-kappa B in response to inflammatory mediators and has been shown to be up-regulated by different extracellular signals, such as granulocyte-macrophage colony-stimulating factor (GM-CSF), CD40, phorbol ester and inflammatory cytokine TNF and IL-1, which suggests a cytoprotective function that is essential for lymphocyte activation as well as cell survival. BCL2A1 is overexpressed in a variety of cancer cells, including hematological malignancies and solid tumors, and may contribute to tumor progression (see, e.g., Vogler, M. BCL2A1: the underdog in the BCL2 family. Cell Death Differ 19, 67-74 (2012)). BCL2A1 exerts its anti-apoptotic function by sequestering pro-apoptotic B-cell lymphoma 2 (BCL2) proteins. Id. Enhanced expression of BCL2A1 can result in resistance to chemotherapeutic drugs. Id. The main function of the anti-apoptotic BCL2 proteins is to counteract the activation of BAX and BAK. Thus, they can either inhibit BAX and BAK directly, or sequester and inactivate BH3-only proteins. Id. Multiple anti-apoptotic BCL2 proteins have been described, namely BCL2, BCL-XL, BCL-w, MCL1, BCL-B and BCL2A1. Id. BCL2A1 does not display a well-defined C-terminal transmembrane domain. The C-terminus of BCL2A1 is of importance for the anti-apoptotic function and the subcellular localization of BCL2A1. Id. BCL2A1 has a similar binding profile as displayed by MCL1. Id. Applicants identified BCL2A1 as a gene overexpressed in tumors resistant to T cell killing. Thus, inhibition of BCL2A1 can be used to enhance anti-tumor immunity and immunotherapy.

Accordingly, in some embodiments disclosed herein, are methods designed to downregulate BCL2A1 expression or inhibit BCL2A1 for treating cancer, preferably in combination with an immunotherapy. In certain embodiments, subjects being treated with an immunotherapy have increased expression of BCL2A1 and become non-responsive. In certain embodiments, administering an agent targeting BCL2A1 expression or activity shifts a subject non-responsive to an immunotherapy to a responder. In certain embodiments, the one or more therapeutics described herein are administered to a subject that has a tumor overexpressing BCL2A1. The present invention also provides for determining subjects that may respond to an BCL2A1 inhibitor. For example, the tumor overexpresses BCL2A1 and does not overexpress another protein that allows evasion of an immune response.

### Small Molecule and Peptide Inhibitors of BCL2A1

Dysregulation of the mitochondrial apoptotic pathway controlled by members of the BCL-2 protein family plays a central role in cancer development and resistance to conventional cytotoxic as well as targeted therapies. The selective inhibition of the pro-survival BCL-2 family of proteins to activate apoptosis in malignant cells represents an anti-cancer strategy. The remarkable clinical performance of the selective BCL-2 antagonist Venetoclax has highlighted the potential for selective inhibitors of the other pro-survival members of the BCL-2 family, particularly BCL2A1. Venetoclax is a BH3-mimetic and can block the anti-apoptotic B-cell lymphoma-2 (Bcl-2) family proteins. The present invention also provides for use of the small molecules/peptide inhibitors in combination with an immunotherapy treatment regimen. In certain embodiments, inhibitors are administered in doses that do not induce apoptosis in the absence of an effective anti-tumor immune response. Thus, doses required to shift a subject from a nonresponder to a responder are lower than doses required to induce apoptosis and the doses of the present invention are lower than certain standard doses. In particular, doses are lower in a combination immunotherapy treatment regimen.

In certain embodiments disclosed herein, the methods provide several drugs or small molecules that are capable of inhibiting the expression or activity of BCL2A1. Non-limiting examples include Venetoclax, AT101 (R-(−)-gossypol), TW-37, Gambogic acid, Sabutoclax (BI-97C1) and Marinopyrrole A (maritoclax). Other small molecules may be used (see, e.g., Vogler, M. BCL2A1: the underdog in the BCL2 family. Cell Death Differ 19, 67-74 (2012)). For example, N-aryl maleimides have been identified as potential BCL2A1 inhibitors by high-throughput screening of 66 000 compounds (Cashman J R, MacDonald M, Ghirmai S, Okolotowicz K J, Sergienko E, Brown B et al. Inhibition of Bfl-1 with N-aryl maleimides. Bioorg Med Chem Lett 2010; 20: 6560-6564). Several apogossypol derivatives may target BCL2A1 (Wei J, Kitada S, Rega M F, Emdadi A, Yuan H, Cellitti J et al. Apogossypol derivatives as antagonists of antiapoptotic Bcl-2 family proteins. Mol Cancer Ther 2009; 8: 904-913). Additionally, peptide aptamers that specifically target BCL2A1 have been presented, which sensitized malignant B-cells to chemotherapeutic drugs (Brien G, Debaud A L, Bickle M, Trescol-Biemont M C, Moncorge O, Colas P et al. Characterization of peptide aptamers targeting Bfl-1 anti-apoptotic protein. Biochemistry 2011; 50: 5120-5129).

### Alternative Methods to Counter-Acting MCL1 and BCL2A1 Anti-Apoptotic Activity in Tumor Cells

The members of the Bcl-2 family are designated as such due to their BCL-2 homology (BH) domains and involvement in apoptosis regulation. The BH domains facilitate the family members' interactions with each other, and can indicate pro- or anti-apoptotic function. Traditionally, these proteins are categorized into one of three subfamilies; anti-apoptotic, BH3-only (pro-apoptotic), and pore-forming or ‘executioner’ (pro-apoptotic) proteins (BAX and BAK) (see, e.g., Warren C F A, Wong-Brown M W, Bowden N A. BCL-2 family isoforms in apoptosis and cancer. Cell Death Dis. 2019; 10(3):177; and Vogler, M. BCL2A1: the underdog in the BCL2 family. Cell Death Differ 19, 67-74 (2012)). MCL-1 exerts its antiapoptotic function by sequestering proapoptotic proteins BAK/BAX through the BH3 domain containing hydrophobic groove. In the prosurvival mode, BAK/BAX interacts with antiapoptotic BCL-2 proteins and is unable to execute the apoptotic program, thereby allowing cells to maintain homeostasis (see, e.g., Xiang W, Yang C Y, Bai L. MCL-1 inhibition in cancer treatment. Onco Targets Ther. 2018; 11:7301-7314). BH3-only proteins, BIM, PUMA, BAD, NOXA and BID, restore BAX/BAK activities through interruption of the MCL-1:BAK/BAX complexes. Id. BAK/BAX allows release of cytochrome c and activates caspase cascade.

### Gene Therapy Embodiments For Increasing BID, PMAIP1, BAX, BAK, BIM, BAD and PUMA

In one example embodiment, gene therapy may be used to increase one or more genes selected from the group consisting of BID, PMAIP1 (NOXA), BAX, BAK, BIM, BAD and PUMA in tumor cells. In certain embodiments, expression is increased in proportion to overexpression of MCL1. In certain embodiments, gene therapy is used for subjects having tumors that overexpress MCL1. As used herein, the terms “gene therapy,” “gene delivery,” “gene transfer” and “genetic modification” are used interchangeably and refer to modifying or manipulating the expression of a gene to alter the biological properties of living cells for therapeutic use.

In one example embodiment, a vector for use in gene therapy comprises a sequence encoding a gene selected from the group consisting of BID, PMAIP1 (NOXA), BAX, BAK, BIM, BAD and PUMA and is used to deliver said sequence to tumor cells. The vector may further comprise one or more regulatory elements to control expression of the gene. The vector may further comprise regulatory/control elements, e.g., promoters, enhancers, introns, polyadenylation signals, Kozak consensus sequences, or internal ribosome entry sites (IRES). The vector may further comprise a targeting moiety that directs the vector specifically to tumor cells. In another example embodiment, the vector may comprise a viral vector with a tropism specific for tumors.

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. There are no limitations regarding the type of vector that can be used. The vector can be a cloning vector, suitable for propagation and for obtaining polynucleotides, gene constructs or expression vectors incorporated to several heterologous organisms. Suitable vectors include eukaryotic expression vectors based on viral vectors (e.g. adenoviruses, adeno-associated viruses as well as retroviruses and lentiviruses), as well as non-viral vectors such as plasmids.

In one example embodiment, the vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operably linked. Such vectors are referred to herein as “expression vectors.” Vectors for and that result in expression in a eukaryotic cell can be referred to herein as “eukaryotic expression vectors.” In another example embodiment, the vector integrates the gene into the cell genome or is maintained episomally.

In one example embodiment, the vector is a “plasmid,” which refers to a circular double stranded DNA loop into which additional DNA segments can be inserted, such as by standard molecular cloning techniques.

In one example embodiment, the vector is an mRNA vector (see, e.g., Sahin, U, Kariko, K and Tureci, O (2014). mRNA-based therapeutics—developing a new class of drugs. Nat Rev Drug Discov 13: 759-780; Weissman D, Kariko K. mRNA: Fulfilling the Promise of Gene Therapy. Mol Ther. 2015; 23(9): 1416-1417. doi: 10.1038/mt.2015.138; Kowalski P S, Rudra A, Miao L, Anderson D G. Delivering the Messenger: Advances in Technologies for Therapeutic mRNA Delivery. Mol Ther. 2019; 27(4):710-728. doi:10.1016/j.ymthe.2019.02.012; Magadum A, Kaur K, Zangi L. mRNA-Based Protein Replacement Therapy for the Heart. Mol Ther. 2019; 27(4): 785-793. doi:10.1016/j.ymthe.2018.11.018; Reichmuth A M, Oberli M A, Jaklenec A, Langer R, Blankschtein D. mRNA vaccine delivery using lipid nanoparticles Ther Deliv. 2016; 7(5):319-334. doi:10.4155/tde-2016-0006; and Khalil A S, Yu X, Umhoefer J M, et al. Single-dose mRNA therapy via biomaterial-mediated sequestration of overexpressed proteins. Sci Adv. 2020; 6(27):eaba2422). In an exemplary embodiment, mRNA encoding for dominant negative JUNB is delivered using lipid nanoparticles (see, e.g., Reichmuth, et al., 2016) and administered directly to a tumor. In an exemplary embodiment, mRNA encoding for dominant negative JUNB is delivered using biomaterial-mediated sequestration (see, e.g., Khalil, et al., 2020) and administered directly to tumor tissue. Sequences present in mRNA molecules, as described further herein, are applicable to mRNA vectors (e.g., Kozak consensus sequence, miRNA target sites and WPRE).

### Regulatory Elements

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operably-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). The term “operably linked” as used herein also refers to the functional relationship and position of a promoter sequence relative to a polynucleotide of interest (e.g., a promoter or enhancer is operably linked to a coding sequence if it affects the transcription of that sequence). Typically, an operably linked promoter is contiguous with the sequence of interest. However, enhancers need not be contiguous with the sequence of interest to control its expression. The term “promoter”, as used herein, refers to a nucleic acid fragment that functions to control the transcription of one or more polynucleotides, located upstream of the polynucleotide sequence(s), and which is structurally identified by the presence of a binding site for DNA-dependent RNA polymerase, transcription initiation sites, and any other DNA sequences including, but not limited to, transcription factor binding sites, repressor, and activator protein binding sites, and any other sequences of nucleotides known in the art to act directly or indirectly to regulate the amount of transcription from the promoter. A “tissue-specific” promoter is only active in specific types of differentiated cells or tissues.

In another embodiment, the vector of the invention further comprises expression control sequences including, but not limited to, appropriate transcription sequences (i.e. initiation, termination, promoter, and enhancer), efficient RNA processing signals (e.g. splicing and polyadenylation (polyA) signals), sequences that stabilize cytoplasmic mRNA, sequences that enhance translation efficiency (i.e. Kozak consensus sequence), and sequences that enhance protein stability. A great number of expression control sequences, including promoters which are native, constitutive, inducible, or tissue-specific are known in the art and may be utilized according to the present invention.

In another embodiment, the vector of the invention further comprises a post-transcriptional regulatory region. In a preferred embodiment, the post-transcriptional regulatory region is the Woodchuck Hepatitis Virus post-transcriptional region (WPRE) or functional variants and fragments thereof and the PPT-CTS or functional variants and fragments thereof (see, e.g., Zufferey R, et al., J. Virol. 1999; 73:2886-2892; and Kappes J, et al., WO 2001/044481). In a particular embodiment, the post-transcriptional regulatory region is WPRE. The term “Woodchuck hepatitis virus posttranscriptional regulatory element” or “WPRE”, as used herein, refers to a DNA sequence that, when transcribed, creates a tertiary structure capable of enhancing the expression of a gene (see, e.g., Lee Y, et ah, Exp. Physiol. 2005; 90(1):33-37 and Donello J, et al, J. Virol. 1998; 72(6):5085-5092).

The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990).

Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as adipose tissue or particular cell types (e.g., adipocytes or adipocyte progenitors). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Also encompassed by the term “regulatory element” are enhancer elements (e.g., adipose specific enhancers or Woodchuck Hepatitis Virus Posttranscriptional Regulatory Element (WPRE)). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., JUNB).

In one embodiment, the vector contains at least one target sequence of at least one miRNA expressed in non-tumor tissue. The term “microRNAs” or “miRNAs”, as used herein, are small (˜22-nt), evolutionarily conserved, regulatory RNAs involved in RNA-mediated gene silencing at the post-transcriptional level (see, e.g., Barrel D P. Cell 2004; 116: 281-297). Through base pairing with complementary regions (most often in the 3′ untranslated region (3′UTR) of cellular messenger RNA (mRNA)), miRNAs can act to suppress mRNA translation or, upon high-sequence homology, cause the catalytic degradation of mRNA. Because of the highly differential tissue expression of many miRNAs, cellular miRNAs can be exploited to mediate tissue-specific targeting of gene therapy vectors. By engineering tandem copies of target elements perfectly complementary to tissue-specific miRNAs (miRT)

### Method of Enhancing Anti-Tumor Immunity Through Targeting of JunB Protein Activity

In one example embodiment, a method of enhancing anti-tumor immunity comprising inhibiting JunB activity. Inhibition of JunB expression or activity may increase the sensitivity of a tumor to T cell killing and further enhances the effectiveness of an immunotherapy (e.g., adoptive cell transfer). JunB inhibitors may be administered to subjects having a cancer with increased expression of JunB, especially where JunB expression is higher as compared to other Jun or AP-1 proteins. Administering a JunB inhibitor may help make a subject who is initially non-responsive to immunotherapy responsive to immunotherapy. The inhibitor used may directly JunB directly or indirection via a downstream target of JunB (Table 5 or Table 6).

JunB is a member of the AP-1 (activator protein-1) family of dimeric transcription factors. The AP-1 family of transcription factors is composed of homodimers and heterodimers of Jun (v-Jun, c-Jun, JunB, and JunD), Fos (v-Fos, c-Fos, FosB, Fra1, and Fra2), ATF (ATF2, ATF3/LRF1, B-ATF, JDP1, and JDP2), and MAF (c-Maf, MafB, MafA, MafG/F/K, and Nrl) protein families, which are characterized by highly conserved dimeric basic leucine zipper (bZIP) DNA-binding domains (see, e.g., Ye N, Ding Y, Wild C, Shen Q, Zhou J. Small molecule inhibitors targeting activator protein 1 (AP-1). J Med Chem. 2014; 57(16):6930-6948). Specific Jun (JunB and JunD) and Fos (FosB, Fra1 and Fra2) proteins have been identified as oncoproteins, all of which are components of AP-1. Id. Certain AP-1 proteins have either a weak transforming activity (Fra1 and Fra2) or no transforming activity (JunB and JunD) due to the lack of potent transactivation domains. Id. AP-1 activity in cancer seems to depend upon AP-1 dimer composition and tumor type as well as its differentiation state, tumor stage, and the genetic background of tumor. Id. Generally, c-Jun mainly has oncogenic functions, while JunB and JunD have antioncogenic effects. Id. As such, it would be quite challenging for medicinal chemists to design potent and specific AP-1 inhibitors as potential therapy for cancer. Id. Individualized treatment may provide a solution to this problem by selecting appropriate patient populations (e.g., overexpressing JunB). In certain embodiments, subjects are treated that overexpress JunB.

### Small Molecule Inhibitors of JunB

In one example embodiment, anti-tumor immunity is enhanced by administering one or more AP-1 inhibitors. Non-limiting examples of AP-1 inhibitors include curcumin, SP100030, SPC-839, T-5224, K1115A, momordin I, isosteviol and analogues thereof.

### Dominant Negative JunB

In certain embodiments, a recombinant JunB dominant negative protein is used to inhibit endogenous JunB activity. JUNB has 347 amino acids with a predicted molecular weight 35.9 kD (see, e.g., HGNC: 6205, NCBI Entrez Gene: 3726, Ensembl: ENSG00000171223, UniProtKB/Swiss-Prot: P17275). Structurally, JUNB is similar to c-JUN, which contains a JNK (MAPK8) docking site (JUNB a.a. 35-61), nuclear localization signal (276-289), basic domain for DNA binding (250-276) and a leucine zipper domain for dimerization (296-317). However, JUNB does not contain a JNK phosphorylation site. Thus, the transactivation activity of JUNB is not regulated by JNK.

JUNB is a member of JUN family (c-JUN, JUNB and JUND) that can dimerize with one another, or with members of Fos and ATF families, to form an AP-1 transcription factor. Comparing with c-JUN, the transactivation activity of JUNB is much weaker (see, e.g., Deng T, Karin M. JunB differs from c-Jun in its DNA-binding and dimerization domains and represses c-Jun by formation of inactive heterodimers. Genes Dev. 1993; 7(3):479-490). Due to the small differences on the amino acid sequences in the basic DNA binding domain, and leucine zipper domain, JUNB requires multiple AP-1 DNA binding sites for sufficient DNA binding. A number of studies demonstrated that JUNB antagonizes the functions of c-JUN in cell cycle regulation, proliferation and transformation by competing with c-JUN to form less efficient transactivating dimers. Thus, JUNB was considered as a tumor suppressor.

In gene knockout studies, mice lacking c-Jun gene die during embryonic day 12.5 and 13.5, whereas embryos lacking JunB die earlier, around day 9.5, owing to vascular defects in the placenta and extraembryonic tissue. Interestingly, gene knock-in experiment indicated that JUNB could partially substitute the activities of c-JUN in mouse development and cell proliferation. As a possible explanation for this is that in presence of c-JUN, JUNB is a negative regulator for c-JUN. In contrast, in the absence of c-JUN, JUNB may substitute c-JUN and activate AP-1 target genes required for development and cell proliferation.

Dominant negative mutants of c-Jun have been described (see, e.g., Brown P H, Kim S H, Wise S C, Sabichi A L, Birrer M J. Dominant-negative mutants of c-Jun inhibit AP-1 activity through multiple mechanisms and with different potencies. Cell Growth Differ. 1996; 7(8): 1013-1021). A dominant-negative mutant of c-Jun that lacks the transactivation domain (TAD) prevents AP-1-mediated transcriptional activation by quenching endogenous Jun or Fos proteins (del. 3-102; and del. 3-122). Id. c-Jun mutations in the TAD, DNA-binding domain (DBD), or leucine zipper domain are all unable to activate transcription, but only TAD and DBD mutants function in a dominant-negative fashion by inhibiting both c-Jun-induced transcriptional activation and transformation. Id. TAM-67 is a c-JUN dominant negative mutant with a deletion of amino acids 3-122 (see, e.g., Matthews C P, Birkholz A M, Baker A R, et al. Dominant-negative activator protein 1 (TAM67) targets cyclooxygenase-2 and osteopontin under conditions in which it specifically inhibits tumorigenesis. Cancer Res. 2007; 67(6):2430-2438).

In certain embodiments, a dominant negative JUNB is administered to a subject. In certain embodiments, the TAD of JUNB is removed or mutated to abolish activity. In certain embodiments, the DBD of JUNB is removed or mutated to abolish activity. In certain embodiments, the dominant negative JUNB is administered directly to a tumor. In certain embodiments, a vector having tropism for tumor cells is administered.

In one example embodiment, a subject in need thereof is treated by expression of a dominant negative JUNB using a gene therapy approach as described herein (see, MCL1). In one example embodiment, a vector for use in gene therapy comprises a sequence encoding dominant negative JUNB and is used to deliver said sequence to tumor cells. Vector based embodiments to deliver polynucleotides to delivery dominant negative JUNB may be used as described above at [0078]-[0084].

### Recombinant Dominant Negative JUNB

In certain embodiments, recombinant dominant negative JUNB protein is delivered intracellularly to a subject in need thereof and is used as a protein therapeutic. Protein therapeutics offer high specificity, and the ability to treat “undruggable” targets, in diseases associated with protein deficiencies or mutations.

In certain embodiments, virus like particles (VLPs) are used to facilitate intracellular recombinant protein therapy (see, e.g., WO2020252455A1, U.S. Ser. No. 10/577,397B2). In certain embodiments, VLPs include a Gag-dominant negative JUNB fusion protein. The Gag-dominant negative JUNB fusion protein may include a matrix protein, a capsid protein, and/or a nucleocapsid protein covalently linked to JUNB. In certain embodiments, the VLPs include a membrane comprising a phospholipid bilayer with one or more human endogenous retrovirus (HERV) derived ENV/glycoprotein(s) on the external side; a HERV-derived GAG protein in the VLP core, and a JUNB fusion protein on the inside of the membrane, wherein JUNB is fused to a human-endogenous GAG or other plasma membrane recruitment domain (see, e.g., WO2020252455A1). Fusion proteins can be obtained using standard recombinant protein technology.

In certain embodiments, cell-penetrating peptides (CPPs) are used to facilitate intracellular recombinant protein therapy (see, e.g., Dinca A, Chien W-M, Chin M T. Intracellular Delivery of Proteins with Cell-Penetrating Peptides for Therapeutic Uses in Human Disease. International Journal of Molecular Sciences. 2016; 17(2):263). In certain embodiments, cell-penetrating peptides can be conjugated to JUNB, for example, using standard recombinant protein technology. In certain embodiments, cell-penetrating peptides can be concurrently delivered with recombinant JUNB.

In certain embodiments, nanocarriers are used to facilitate intracellular recombinant protein therapy (see, e.g., Lee Y W, Luther D C, Kretzmann J A, Burden A, Jeon T, Zhai S, Rotello V M. Protein Delivery into the Cell Cytosol using Non-Viral Nanocarriers. Theranostics 2019; 9(11):3280-3292). Non-limiting nanocarriers include, but are not limited to nanoparticles (e.g., silica, gold), polymers, lipid based (e.g., cationic lipid within a polymer shell, lipid-like nanoparticles).

### Method of Inhibiting B3GNT2, MCL1, BCLA21, and JunB Expression By RNAi

In one example embodiment, a method of enhancing anti-tumor activity comprises administering a RNAi therapeutic to reduce expression of one or more of B3GNT2, MCL1, BCLA21, and JUNB (collectively “target sequences”). A RNAi therapeutic comprises a polynucleotide that is complementary to a portion of the target sequence mRNA, generally ranging in size from 15 to 50 base pairs. RNAi modalities may include miRNA and siRNA. The RNAi modality may also be in the form a pre-miRNA which is processed by Dicer to form a miRNA. Likewise, the RNAi modality may be in the form of a dsRNA or shRNA which is processed by Dicer to form a siRNA. RNAi modalities may also be derived from endogenous microRNA. The RNAi polynucleotide may comprise one or more modifications to suppress innate immune activation, enhance activity and specificity, and reduce off-target induced toxicity. The RNAi therapeutic may further comprise a delivery platform for delivery of the RNAi polynucleotide.

The RNAi modalities used herein may be used to achieve gene silencing of B3GNT2 expression. As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

### Example Polynucleotide Sequences

The following sections provide example target sequences to which RNAi polynucleotides may be designed to hybridize to and induce RNAi mediated knockdown of target gene expression.

### B3GNT2 Sequences

B3GNT2, also known as 3-Gn-T1, 3-Gn-T2, B3Gn-T2, B3GNT, B2GNT-2, B3GNT1 BETA3GNT, BGNT2, BGnT-2, beta-1 beta-3Gn-T1, and beta3Gn-T2 is located on human chromosome 2, locus 2P15, accession No. NC_000002.12 from position 62196115 to 62/224,731. In one example embodiment, the polynucleotide sequence included in the vector is a DNA sequence with the primary accession numbers AC018462, AC093401 and CH471053. In another example embodiment, the DNA sequence is selected from the group consisting of AC018462, AC093401 and CH471053.

In another example embodiment, the polynucleotide sequence included in the vector is a RNA sequence derived from NM_006577.6 and NM_001319075.2. In another example embodiment, the polynucleotide sequence included in the vector is a RNA sequence selected from the group consisting of NM_006577.6 and NM_001319075.2. In another example embodiment, the sequence included in the vector is derived from mRNA selected from the group consisting of AB049584.1, AF09205.12, AF288208.1, AF288209.1, AJ006077.1, AK002009.1, BC030579.2 and BC047933.1. In another example embodiment, the sequence included in the vector is a mRNA sequence selected from the group consisting of AB049584.1, AF09205.12, AF288208.1, AF288209.1, AJ006077.1, AK002009.1, BC030579.2 and BC047933.1. In another example embodiment, the amino acid sequence is derived from the primary accession number Q9NY97, NP_006568.2, and NP_001306004.1. In another example embodiment, the amino acid sequence is selected from the group consisting of Q9NY97, NP_006568.2 and NP_001306004.1. In another example embodiment, the amino acid sequence is derived from the secondary accession numbers Q54AC1, Q9NQQ9, Q9NQR0 and Q9NUT9. In another example embodiment, the amino acid sequence is selected from the group consisting of Q54AC1, Q9NQQ9, Q9NQR0 and Q9NUT9.

### MCL1 Sequences

MCL1, also known as TM; EAT; MCL1L; MCL1S; Mcl-1; BCL2L3; MCL1-ES; bcl2-L-3; mcl1/EAT is located on human chromosome 1, locus 1Q21.2, accession no. NC_000001.11 from position, complement 150547034 to 150552086. In one example embodiment, the polynucleotide sequence included in the vector is a DNA sequence with the primary accession numbers AF147742.1, AF198614.1, AH009713.2, AL356356.17, CH471121.2 and NG_029146. In another example embodiment, the DNA sequence is selected from the group consisting of AF147742.1, AF198614.1, AH009713.2, AL356356.17, CH471121.2 and NG_029146.

In another example embodiment, the polynucleotide sequence included in the vector is a RNA sequence derived from NM_021960.5, NM_182763.3 and NM_001197320.2. In another example embodiment, the polynucleotide sequence included in the vector is a RNA sequence selected from the group consisting of NM_021960.5, NM_182763.3 and NM 001197320.2. In another example embodiment, the sequence included in the vector is derived from mRNA selected from the group consisting of AA453505.1, AF118124.1, AF118276.1, AF118277.1, AF118278.1, AK294462.1, AK297217.2, AK300499.1, AK304775.1, AK312508.1, AK316267.1, BC017197.2, BC071897.1, BC107735.1, BT006640.1, CA421486.1, FJ917536.1 and L08246.1. In another example embodiment, the sequence included in the vector is a mRNA sequence selected from the group consisting of AA453505.1, AF118124.1, AF118276.1, AF118277.1, AF118278.1, AK294462.1, AK297217.2, AK300499.1, AK304775.1, AK312508.1, AK316267.1, BC017197.2, BC071897.1, BC107735.1, BT006640.1, CA421486.1, FJ917536.1 and L08246.1. In another example embodiment, the amino acid sequence is derived from the primary accession number Q07820, NP_068779.1, NP_877495.1 and NP_001184249.1. In another example embodiment, the amino acid sequence is selected from the group consisting of Q07820, NP_068779.1, NP_877495.1 and NP_001184249.1. In another example embodiment, the amino acid sequence is derived from the secondary accession numbers B2R6B2, D3DV03, D3DV04, Q9HD91, Q9NRQ3, Q9NRQ4, Q9UHR7, Q9UHR8, Q9UHR9 and Q9UNJ1. In another example embodiment, the amino acid sequence is selected from the group consisting of B2R6B2, D3DV03, D3DV04, Q9HD91, Q9NRQ3, Q9NRQ4, Q9UHR7, Q9UHR8, Q9UHR9 and Q9UNJ1.

### BCLA21 Sequences

BCL2A1, also known as ACC-1, ACC-2, ACC1, ACC2, BCL2L5, BFL1, GRS, HBPA1 is located on human chromosome 15, locus 15Q25.1, accession no. NC_000015.10 from position, complement 79960892 to 79971196. In one example embodiment, the polynucleotide sequence included in the vector is a DNA sequence with the primary accession numbers AC015871.7, AF479683.1, CH471136.2, DQ081729.1, HI574046.1 and NG_029487. In another example embodiment, the DNA sequence is selected from the group consisting of AC015871.7, AF479683.1, CH471136.2, DQ081729.1, HI574046.1 and NG_029487.

In another example embodiment, the polynucleotide sequence included in the vector is a RNA sequence derived from NM_004049.4, and NM_001114735.2. In another example embodiment, the polynucleotide sequence included in the vector is a RNA sequence selected from the group consisting of NM_004049.4, and NM_001114735.2. In another example embodiment, the sequence included in the vector is derived from mRNA selected from the group consisting of AL110097.1, AY234180.1, BC016281.1, BG198875.1, BG204033.1, BG216703.1, BT007103.1, CD640106.1, CR541937.1, CR541962.1, U27467.1, U29680.1 and Y09397.1. In another example embodiment, the sequence included in the vector is a mRNA sequence selected from the group consisting of AL110097.1, AY234180.1, BC016281.1, BG198875.1, BG204033.1, BG216703.1, BT007103.1, CD640106.1, CR541937.1, CR541962.1, U27467.1, U29680.1 and Y09397.1. In another example embodiment, the amino acid sequence is derived from the primary accession number Q16548, NP_068779.1, NP_877495.1 and NP_001184249.1. In another example embodiment, the amino acid sequence is selected from the group consisting of Q16548, NP_068779.1, NP 877495.1 and NP_001184249.1. In another example embodiment, the amino acid sequence is derived from the secondary accession numbers Q6FGZ4, Q6FH19, Q86W13 and Q99524. In another example embodiment, the amino acid sequence is selected from the group consisting of Q6FGZ4, Q6FH19, Q86W13 and Q99524.

### JUNB Sequences

JUNB, also known as AP-1 is located on human chromosome 19, locus 19P13.13, accession no. NC_000019.10 from position 12791486 to 12793315. In one example embodiment, the polynucleotide sequence included in the vector is a DNA sequence with the primary accession numbers AC01861.6, AY751746.1, CH471106.1, KT584920.1, M29039.1 and U20734. In another example embodiment, the DNA sequence is selected from the group consisting of AC01861.6, AY751746.1, CH471106.1, KT584920.1, M29039.1 and U20734.

In another example embodiment, the polynucleotide sequence included in the vector is a RNA sequence derived from NM_002229.3. In another example embodiment, the polynucleotide sequence included in the vector is a RNA sequence selected from the group consisting of NM_002229.3. In another example embodiment, the sequence included in the vector is derived from mRNA selected from the group consisting of AK222532.1, AK313737.1, BC004250.1, BC009465.1, BC009466.1, BC1130372.1, BT019760.1, DQ650707.1 and X51345.1. In another example embodiment, the sequence included in the vector is a mRNA sequence selected from the group consisting of AK222532.1, AK313737.1, BC004250.1, BC009465.1, BC009466.1, BC1130372.1, BT019760.1, DQ650707.1 and X51345.1. In another example embodiment, the amino acid sequence is derived from the primary accession number P17275 and NP_002220.1. In another example embodiment, the amino acid sequence is selected from the group consisting of P17275 and NP_002220.1. In another example embodiment, the amino acid sequence is derived from the secondary accession numbers Q96GH3. In another example embodiment, the amino acid sequence is selected from the group consisting of Q96GH3.

All gene name symbols as used throughout the specification refer to the gene as commonly known in the art. The examples described herein that refer to gene names are to be understood to encompass human genes, as well as genes in any other organism (e.g., homologous, orthologous genes). The term, homolog, may apply to the relationship between genes separated by the event of speciation (e.g., ortholog). Orthologs are genes in different species that evolved from a common ancestral gene by speciation. Normally, orthologs retain the same function in the course of evolution. Gene symbols may be those referred to by the HUGO Gene Nomenclature Committee (HGNC) or National Center for Biotechnology Information (NCBI). Any reference to the gene symbol is a reference made to the entire gene or variants of the gene. Reference to a gene encompasses the gene product (e.g., protein encoded for by the gene).

### Example siRNA Embodiments

In one example embodiment, the RNAi modality is a siRNA. As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

### Example shRNA Embodiments

In one example embodiment, the RNAi modality is a shRNA. As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

### Example microRNA Embodiments

In one example embodiment, the RNAi modality is engineered microRNA derived from an endogenous. The terms “microRNA” or “miRNA”, used interchangeably herein, are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

### Example dsRNA Embodiments

In one example embodiment, the RNAi modality is a dsRNA. As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Example RNAi configurations

In an embodiment, a single-stranded RNAi molecule disclosed herein has a single-stranded oligonucleotide structure and mediates RNA interference against a target RNA (e.g., B3GNT2). A single-stranded B3GNT2 RNAi agent comprises: (a) a nucleic acid portion comprising a first nucleotide portion (N1) and a second nucleotide portion (N2), wherein said nucleic acid portion comprises at least 8 nucleotides that can base pair with a target RNA, and wherein the total number of nucleotides within the nucleic acid portion is from 8 to 26 nucleotides; and, (b) an internal spacer portion comprising at least a first non-nucleotide spacer portion (S1) that covalently links the first and second nucleotide portions. The first and second nucleotide portions are not self-complementary. The total number of nucleotides of a single-stranded B3GNT2 RNAi agent disclosed herein (e.g., 8 to 26) is distributed between the nucleotide portions of the RNAi molecule, wherein each nucleotide portion contains at least one nucleotide.

In one embodiment, the nucleic acid portion of a single-stranded B3GNT2 RNAi agent disclosed herein contains two nucleotide portions, referred to as the first nucleotide portion (N1) and the second nucleotide portion (N2). The first and second nucleotide portions of a B3GNT2 RNAi agent disclosed herein are covalently attached to a non-nucleotide spacer portion of the molecule. In another embodiment, the nucleic acid portion of the B3GNT2 single-stranded RNAi agent disclosed herein contains more than one nucleotide portion (e.g., 3, 4, or 5, referred to as third (N3), fourth (N4) or fifth (N5) nucleotide portions, respectively).

In one embodiment, the internal spacer portion of a single-stranded B3GNT2 RNAi agent disclosed herein contains only one non-nucleotide spacer portion, referred to as the first non-nucleotide spacer portion (S1). The first non-nucleotide spacer portion (S1) of a B3GNT2 RNAi agent disclosed herein is covalently attached to two nucleotides and/or non-nucleotide substitutes, each located within a distinct nucleotide portion of the single-stranded molecule. In another embodiment, the internal spacer portion of a single-stranded B3GNT2 RNAi agent disclosed herein contains more than one non-nucleotide spacer portion (e.g., 2, 3, or 4, referred to as second (S2), third (S3) or fourth (S4) non-nucleotide spacer portions, respectively).

A single-stranded B3GNT2 RNAi agent disclosed herein can comprise substitutions, chemically modified nucleotides, and non-nucleotides, including substitutions or modifications in the backbone, sugars, bases, or nucleosides. In certain embodiments, the use of substituted or modified single-stranded B3GNT2 RNAi agents can enable achievement of a given therapeutic effect at a lower dose since these molecules may be designed to have an increased half-life in a subject or biological samples (e.g., blood). Furthermore, certain substitutions or modifications can be used to improve the bioavailability of single-stranded B3GNT2 RNAi agents by targeting particular cells or tissues or improving cellular uptake of the single-stranded B3GNT2 RNAi agents.

In an embodiment, the internal spacer portion of a single-stranded B3GNT2 RNAi agent can comprise one or more non-nucleotide spacer portions. A non-nucleotide spacer portion can include any aliphatic or aromatic chemical group that can be further substituted, wherein said spacer portion does not contain a nucleotide. The spacer portion can be substituted with a chemical moiety that provides additional functionality to a single-stranded B3GNT2 RNAi agent. For example, a non-nucleotide spacer portion can be substituted with a moiety that binds specifically to a target molecule of interest or facilitates/enhances cellular delivery of the molecule. In one embodiment, a non-nucleotide spacer portion includes an alkyl, alkenyl or alkynyl chain of preferably 1 to 20 carbons that can be optionally substituted.

The single-stranded B3GNT2 RNAi molecules disclosed herein are useful agents, which can be used in methods for a variety of therapeutic, diagnostic, genetic engineering, and pharmacological applications. Thus, embodiments of the present disclosure further include methods comprising using a single-stranded B3GNT2 RNAi agent and methods for inhibiting B3GNT2 expression of one or more corresponding target mRNAs to enhance anti-tumor immunity in a cell or organism. Further, this disclosure provides methods and B3GNT2 RNAi agents for treating a subject, by enhancing anti-tumor immunity in a subject in need thereof, including a human cell, tissue, individual or subject.

### Modifications

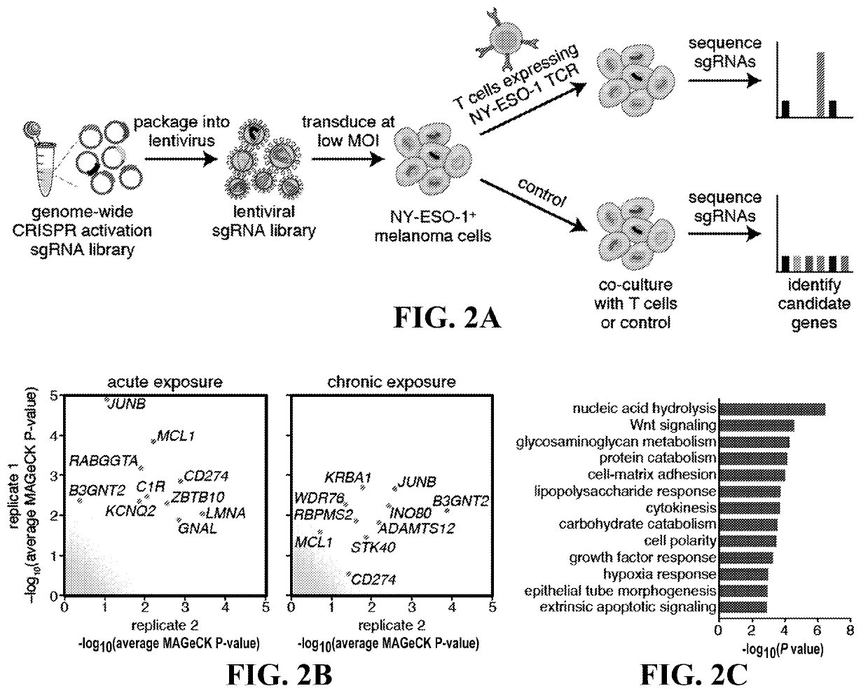

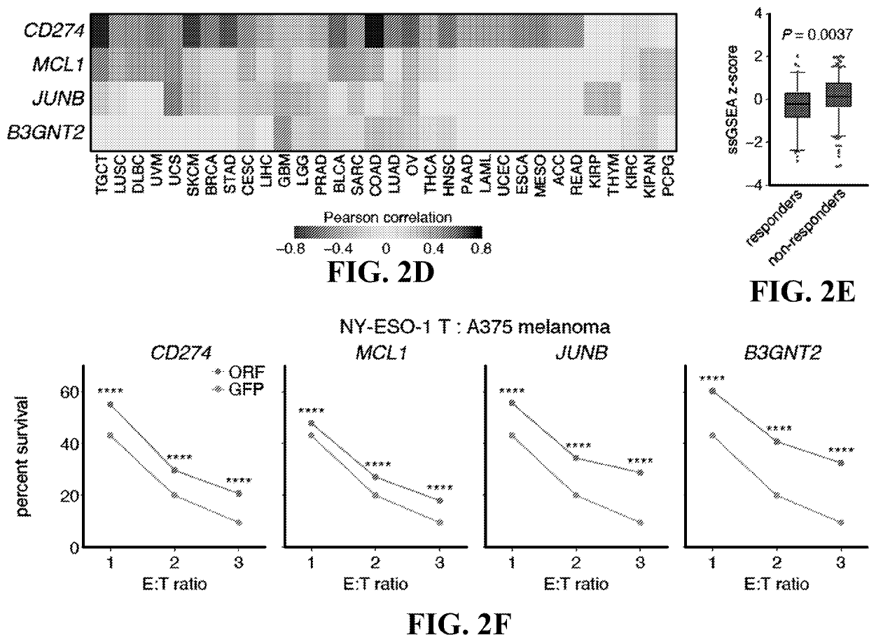

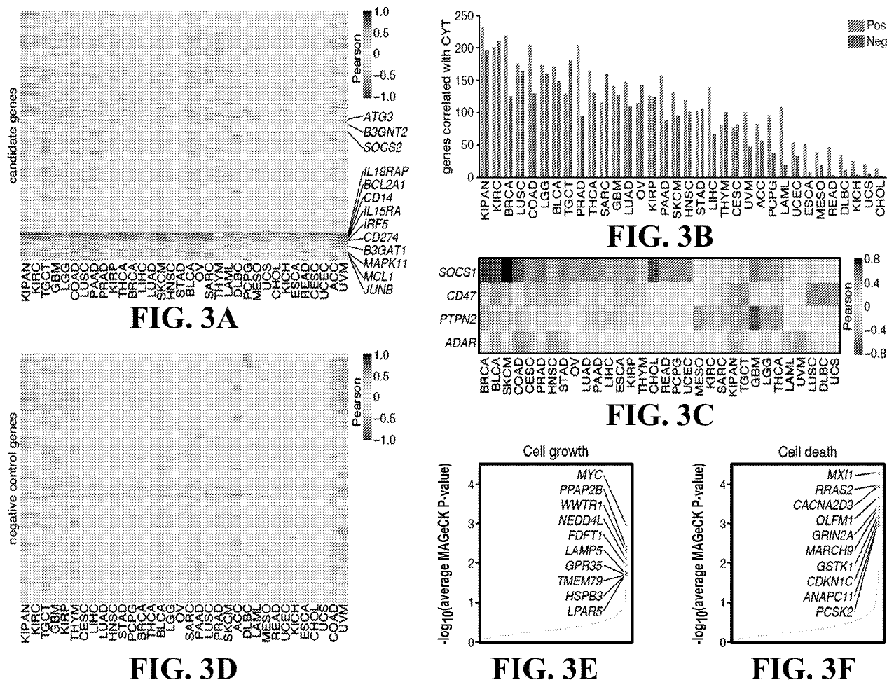

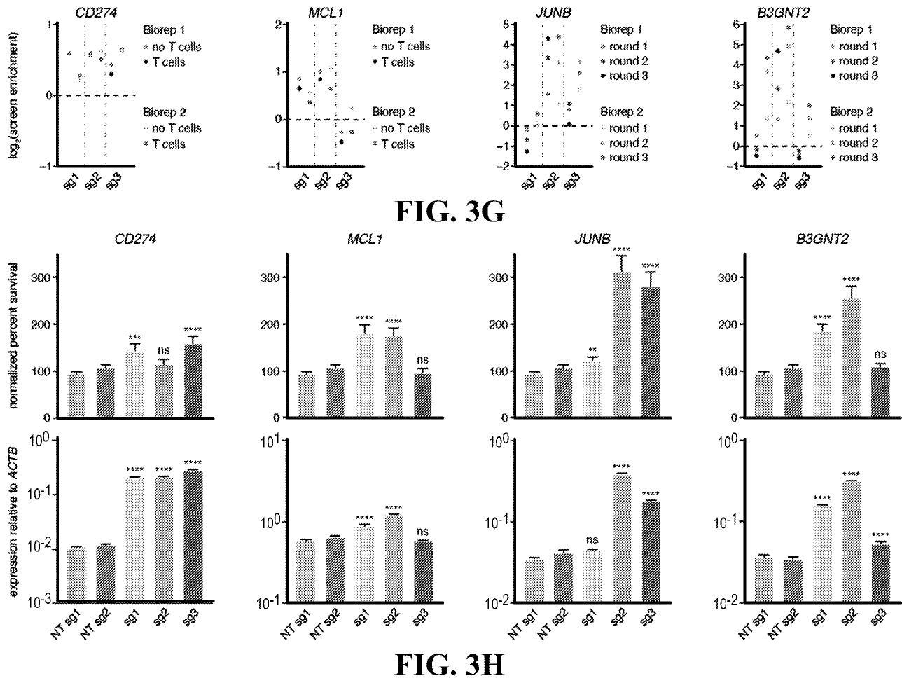

The RNAi modalities described above may comprise one or more modifications including, but not limited to, base modification, ribose modifications, and phosphate modifications. Example base modifications may include 2′-O-methyl, 2′0-methoxyethyl, 2′-arabinoo-fluoro, 2′-O-benzyl, 2′-O-methyl-4-pyridine, locked nucleic acid (LNA), (S)-cEt-BNA, tricyclo-DNA, PMO, unlocked nucleic acid, and glycol nucleic acid. Phosphate modifications include phophoorothioate (PS, Rp isomer, and PS, Sp isomer), phosphorodithioate, methylphosphonate, methoxypropyl-phosphonate, 5′-(E)-vinylphophonate, 5′-Methyl Phosphonate, (S)-5′-C-methyl with phosphate, 5′-phosphorothioate, and peptide nucleic acid. Base modifications may include pseudouridine, 2′-thiouridine, N6′-methyladenosine, 5′-methylcytidine, 5′-fluoro-2′-deoxyuridine, N-ethylpiperidine 7′-EAA triazole modified adenine, N-ethylpiperidine 6′-triazole modified adenine, 6′-phenylpyrrolo-cytosinie, 2′,4′-difluorotoluly ribonucleoside, and 5′-nitroindole. A summary of modifications and example locations within a RNAi polynucleotide for each modification are describe in Hu et al. “Therapeutic siRNA: state of the art” Signal Transduction and Targeted Therapy 5, Article number 100 (2020), particularly FIGS. 2 and 3, which are incorporated herein by reference.

### Delivery Platforms

While the RNAi polynucleotides described above may be delivered as naked RNA (with or without modification) in certain example embodiments, the RNAi therapeutic may further comprise a delivery platform. Example delivery planforms include, but are not limited to liposomes, conjugates, peptides, exosomes, polymers, dendrimers, and inorganic nanoparticles. Example liposomes include Dlin-DMA, Dlin-MC3-DMA, and EnCore.

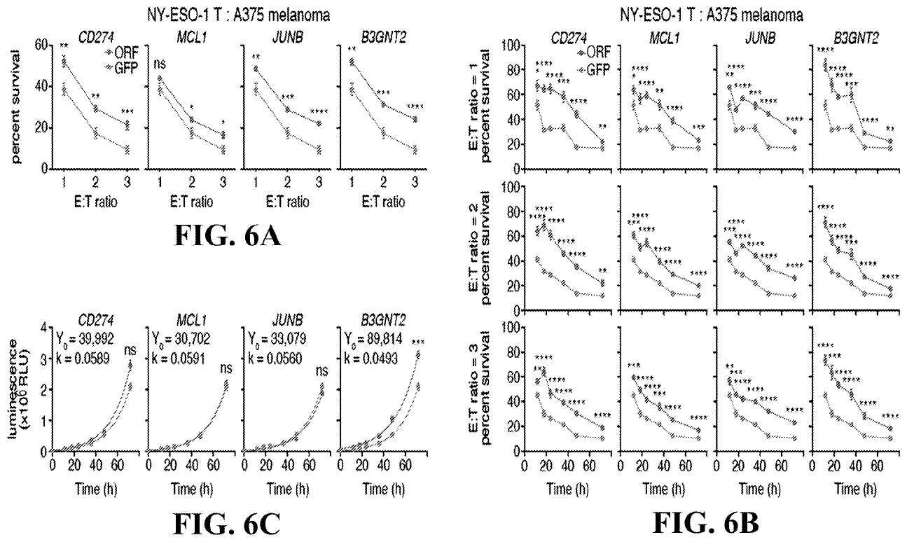

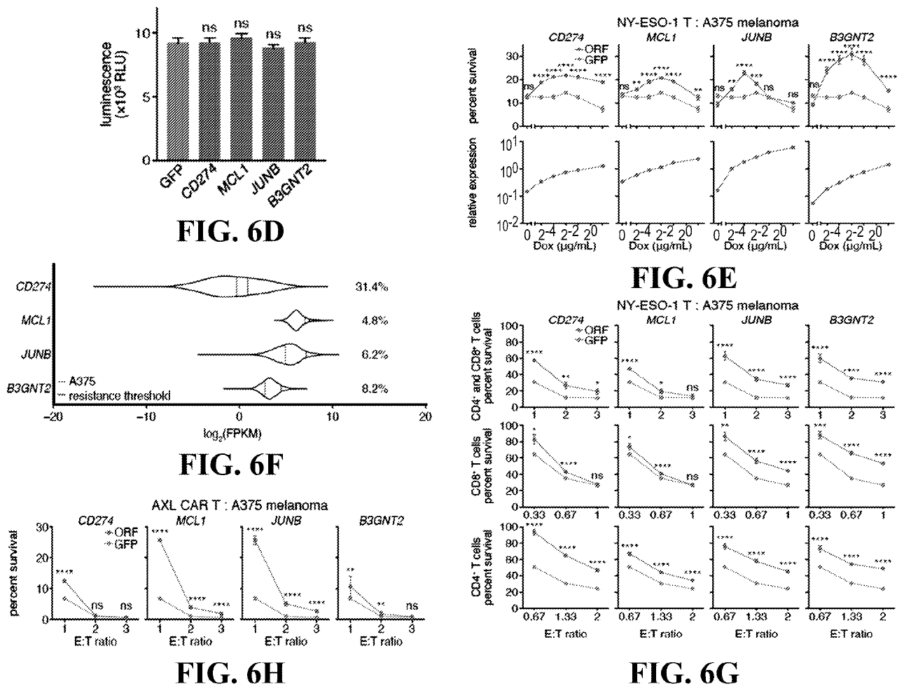

Example conjugates include GalNAc, cholesterol, and RGD. Example polymers include cyclodextrin, PBAVE, PEI, and PLGA. Example peptides include DPC2.0 (MLP), and PNP. Example delivery platforms are described in Hu et al. “Therapeutic siRNA: state of the art” Signal Transduction and Targeted Therapy 5, Article number 100 (2020), particularly pages 11-20 and FIG. 6, which are incorporated herein by reference.

In general, and throughout this specification, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. There are no limitations regarding the type of vector that can be used. The vector can be a cloning vector, suitable for propagation and for obtaining polynucleotides, gene constructs or expression vectors incorporated to several heterologous organisms. Suitable vectors include eukaryotic expression vectors based on viral vectors (e.g. adenoviruses, adeno-associated viruses as well as retroviruses and lentiviruses), as well as non-viral vectors such as plasmids. Exemplary therapeutic delivery vectors of RNAi including viruses are described in Nguyen et al. “RNAi therapeutics: An update on delivery” (2008). Current Opinion in Molecular Therapeutics 10(2): 158-167. Exemplary RNAi delivery vectors from a variety of viruses including, but not limited to, adenovirus (Ad), adeno-associated virus (AAV), retroviruses, et al. are described in Lundstrom, K. “Viral Vectors Applied for RNAi-Based Antiviral Therapy” Viruses (2020) 12, 924 doi: 10:3390/v12092924 (14 pages), particularly on pages 3 and 4, which are herein incorporated by reference. Exemplary viral vectors using alphaviruses, flaviviruses, measles viruses and rhabdoviruses are described in Lundstrom, K. “Self-Amplifying RNA Viruses as RNA Vaccines” 21, 5130 (2020); doi:10.3390/ijms21145130 (29 pages), particularly the viral vectors listed on page 6, which are herein incorporated by reference.

In one example embodiment, the vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). Viral vectors also include polynucleotides carried by a virus for transfection into a host cell. Certain vectors are capable of autonomous replication in a host cell into which they are introduced (e.g., episomal mammalian vectors). Other vectors (e.g., non-episomal mammalian vectors) are integrated into the genome of a host cell upon introduction into the host cell, and thereby are replicated along with the host genome.

In one example embodiment, the vector is a “plasmid,” which refers to a circular double stranded DNA loop into which additional DNA segments can be inserted, such as by standard molecular cloning techniques.

In one example embodiment, RNAi molecules are delivered via liposomes. The RNAi molecules may be modified.

Use of Gene Editing Systems to Reduce B3GNT2, MCL1, BCLA21, and/or JunB Expression or Activity in Tumor Cells

In one example embodiment, a gene editing system is used to reduce the expression or activity of one or more of B3GNT2, MCL1, BCLA21 and JUNB (collectively “target sequences”) in tumor cells. In one example embodiment, a programmable nuclease is used to make one or more insertions or deletions in one or more that target sequences that results in reduced expression of the one or more target sequences. In another example embodiment, a programmable nuclease, in combination with a donor template, is used to replace a portion one or more target genes with either a non-functional variant of the target gene or a modified target gene that results in expression of a gene produce of reduced activity. In one example embodiment, a catalytically inactive programmable nuclease is used to recruit a functional domain (e.g., repressor domain) to the target gene to reduce expression. In one example embodiment, the gene editing system is a base editing system. In one example embodiment, the base editing system is a DNA base editing system used to make one or more base or base pair edits to one or more target genes that reduce target gene expression. In one example embodiment, the base editing system is a RNA base editing system used to modify mRNA expressed from the one or more genes to reduce protein function, for example, by modifying one or more post-translation modification sites encoded by the mRNA In another example embodiment, the gene editing system is a prime editing system. A prime editing system may be used to edit DNA like a base editing system. A prime editing system may also be used to replace all or a portion of the target genes to produce a non-functional variant or expression of a gene product with reduced activity.

### Programmable Nucleases

In certain example embodiments, a programmable nuclease may be used to edit a genomic region comprising one or more genomic variants associated with decreased expression or activity of B3GNT2. Gene editing using programmable nucleases may utilize two different cell repair pathways, non-homologous end joining (NHEJ) and homology-directed repair (HDR). In certain example embodiment, HDR is used to provide a template that replaces a genomic region comprising the variant with a donor that edits the risk variant to a wild-type or non-risk variant. Example programmable nucleases for use in this manner include zinc finger nucleases (ZFN), TALE nucleases (TALENS), meganucleases, and CRISPR-Cas systems.

### CRISPR-Cas

In one example embodiment, the gene editing system is a CRISPR-Cas system. The CRISPR-Cas system comprises a Cas polypeptide and a guide sequence, wherein the guide sequence is capable of forming a CRISPR-Cas complex with the Cas polypeptide and directing site-specific binding of the CRISPR-Cas sequence to a target sequence. The Cas polypeptide may induce a double- or single-stranded break at a designated site in the target sequence. The site of CRISPR-Cas cleavage, for most CRISPR-Cas systems, is dictated by distance from a protospacer-adjacent motif (PAM), discussed in further detail below. Accordingly, a guide sequence may be selected to direct the CRISPR-Cas system to induce cleavage at a desired target site at or near the one or more variants.

### NHEJ-Based Editing

### B3GNT2

In one example embodiment, the CRISPR-Cas system is used to introduce one or more insertions or deletions that reduces or inhibits B3GNT2 expression or activity. More than one guide sequence may be selected to insert multiple insertions, deletions, or combinations thereof. Likewise, more than one Cas protein type may be used, for example, to maximize targets sites adjacent to different PAMs. In one example embodiment, a guide sequence is selected that directs the CRISPR-Cas system to make one or more insertions or deletions in wild type B3GNT2 that reduces B3GNT2 expression. The wild type human B3GNT2 gene, Locus and Accession number NM_006577, is 2761 bp encoding a protein of 397 amino acids. The B3GNT2 gene contains two exons from nt 1-241 and from nt 242 to 2761. The B3GNT2 ORF contains the feature of having an in-frame stop codon at nt 245 to nt 247. The B3GNT2 amino acid sequence contains a transmembrane region from AA 272 to 334, glycosylation sites at AA 485 to 487, 515 to 517, 629 to 631, 767 to 769, 905 to 907, a polyA signal sequence (AATAAA) at AA 2740 to 2745 and a major polyA site at AA 2761. In one example embodiment, a guide is selected that directs the CRISPR-Cas system to make one or more deletions that affects one or more splicing sites in the B3GNT2 gene causing a decrease in the expression of the B3GNT2 ORF. In an embodiment, a guide is selected that directs the CRISPR-Cas system to make a deletion in the B3GNT2 gene such that a frame-shift is introduced into the ORF causing a decrease in expression of the full-length B3GNT2 transcript. In one example embodiment, a guide sequence is selected that directs the CRISPR-Cas system to make an insertion that affects one or more splicing sites in the B3GNT2 gene, which causes a decrease in the expression of the B3GNT2 gene. In an embodiment, a guide is selected that directs the CRISPR-Cas system to make an insertion in the B3GNT2 gene such that a frame-shift is introduced into the ORF causing a decrease in expression of the full-length B3GNT2 transcript.

In one example embodiment, a guide is selected that directs the CRISPR-Cas system to make a deletion that affects the transmembrane region of the B3GNT2 protein, which causes instability and a decrease in the activity of the B3GNT2 protein. In an embodiment, a guide is selected that directs the CRISPR-Cas system to make a deletion in the B3GNT2 ORF such that a frame-shift is introduced into the ORF introducing a stop codon which leads to a decrease in activity of the truncated B3GNT2 protein. In an example embodiment, a guide is selected that directs the CRISPR-Cas system to make a substitution at amino acid position 245 (D, aspartic acid) which abolishes catalytic activity. In one example embodiment, a guide is selected that directs the CRISPR-Cas system to make a deletion or insertion of the B3GNT2 gene, which leads to disruption of the polyA signal sequence region of the protein, causing a decrease in activity of the B3GNT2 protein.

### MCL1

In one example embodiment, the CRISPR-Cas system is used to introduce one or more insertions or deletions that reduces or inhibits MCL1 expression or activity. More than one guide sequence may be selected to insert multiple insertions, deletions, or combinations thereof. Likewise, more than one Cas protein type may be used, for example, to maximize targets sites adjacent to different PAMs. In one example embodiment, a guide sequence is selected that directs the CRISPR-Cas system to make one or more insertions or deletions in wild type MCL1 that reduces MCL1 expression. The wild type human MCL1 gene, Locus and Accession number NM_021960, is 3950 bp encoding a protein of 350 amino acids. The MCL1 ORF contains three exons from nt 1-768, from nt 769 to 1016 and from nt 1017 to 3950. The MCL1 gene contains the feature of having a transcription initiation site at nt 1. The MCL1 amino acid sequence contains functional domains for multiple phosphorylation sites, a PEST-like site, two cleavage sites, a BH3-binding site, a BH2-binding site, a BH1-binding region, a transmembrane region, a polyA signal sequence and a major polyA site. In one example embodiment, a guide is selected that directs the CRISPR-Cas system to make one or more deletions that affects one or more splicing sites in the MCL1 gene causing a decrease in the expression of the MCL1 gene. In an embodiment, a guide is selected that directs the CRISPR-Cas system to make a deletion in the MCL1 gene such that a frame-shift is introduced into the ORF causing a decrease in expression of the full-length MCL1 transcript. In one example embodiment, a guide sequence is selected that directs the CRISPR-Cas system to make one or more insertions that affects one or more splicing sites in the MCL1 gene, which causes a decrease in the expression of the MCL1 gene. In an embodiment, a guide is selected that directs the CRISPR-Cas system to make one or more insertions in the MCL1 gene such that a frame-shift is introduced into the ORF causing a decrease in expression of the full-length MCL1 transcript.

In one example embodiment, a guide is selected that directs the CRISPR-Cas system to make one or more deletions that affects or disrupts one or more phosphorylation sites, one or more cleavage sites, the BH1-, BH2-, BH3-binding sites or the transmembrane region of the MCL1 protein, or any combination thereof, all of which cause protein instability and a decrease in the activity of the MCL1 protein. In an embodiment, a guide is selected that directs the CRISPR-Cas system to make a deletion in the MCL1 ORF such that a frame-shift is introduced into the ORF introducing a stop codon which leads to a decrease in activity of the truncated MCL1 protein. In one example embodiment, a guide is selected that directs the CRISPR-Cas system to make one or more deletions or insertions of the MCL1 gene, which leads to disruption of the polyA signal sequence region of the MCL1 protein, causing a decrease in activity of the MCL1 protein.

### BCL2A1

In one example embodiment, the CRISPR-Cas system is used to introduce one or more insertions or deletions that reduces or inhibits BCL2A1 expression or activity. More than one guide sequence may be selected to insert multiple insertions, deletions, or combinations thereof. Likewise, more than one Cas protein type may be used, for example, to maximize targets sites adjacent to different PAMs. In one example embodiment, a guide sequence is selected that directs the CRISPR-Cas system to make one or more insertions or deletions in wild type BCL2A1 that reduces BCL2A1 expression. The wild type human BCL2A1 gene, Locus and Accession number NM_004049 is 780 bp encoding a protein of 175 amino acids. The BCL2A1 ORF contains two exons from nt 1-497 and from nt 498 to 780. The BCL2A1 ORF contains the feature of having an in-frame stop codon at nt 3 to nt 5. The BCL2A1 amino acid sequence contains a BH2-binding site, a BH1-binding site, a polyA signal sequence and a major polyA site. In one example embodiment, a guide is selected that directs the CRISPR-Cas system to make one or more deletions that affects one or more splicing sites in the BCL2A1 gene causing a decrease in the expression of the BCL2A1 gene. In an embodiment, a guide is selected that directs the CRISPR-Cas system to make a deletion in the BCL2A1 gene such that a frame-shift is introduced into the ORF causing a decrease in expression of the full-length BCL2A1 transcript. In one example embodiment, a guide sequence is selected that directs the CRISPR-Cas system to make one or more insertions that affects one or more splicing sites in the BCL2A1 gene, which causes a decrease in the expression of the BCL2A1 gene. In an embodiment, a guide is selected that directs the CRISPR-Cas system to make one or more insertions in the BCL2A1 gene such that a frame-shift is introduced into the ORF causing a decrease in expression of the full-length BCL2A1 transcript.

In one example embodiment, a guide is selected that directs the CRISPR-Cas system to make one or more deletions that affects the transmembrane region of the BCL2A1 protein, which causes instability and a decrease in the activity of the BCL2A1 protein. In an embodiment, a guide is selected that directs the CRISPR-Cas system to make a deletion in the BCL2A1 ORF such that a frame-shift is introduced into the ORF introducing a stop codon which leads to a decrease in activity of the BCL2A1 protein. In one example embodiment, a guide is selected that directs the CRISPR-Cas system to make one or more deletions or insertions of the BCL2A1 gene which leads to disruption of the polyA signal sequence region of the BCL2A1 protein, causing a decrease in activity of the BCL2A1 protein.

### JUNB

In one example embodiment, the CRISPR-Cas system is used to introduce one or more insertions or deletions that reduces or inhibits JUNB expression or activity. More than one guide sequence may be selected to insert multiple insertions, deletions, or combinations thereof. Likewise, more than one Cas protein type may be used, for example, to maximize targets sites adjacent to different PAMs. In one example embodiment, a guide sequence is selected that directs the CRISPR-Cas system to make one or more insertions or deletions in wild type JUNB that reduces JUNB expression. The wild type human JUNB gene, Locus and Accession number NM_002229 is 1830 bp encoding a protein of 347 amino acids. The JUNB gene contains one exon from nt 1-1830. The JUNB gene contains the feature of having an in-frame stop codon at nt 8 to nt 10. The JUNB amino acid sequence contains multiple phosphorylation sites, an acetylation site, a leucine zipper region a polyA signal sequence and a major polyA site. In one example embodiment, a guide is selected that directs the CRISPR-Cas system to make one or more deletions that affects the splicing site in the JUNB gene causing a decrease in the expression of the JUNB gene. In an embodiment, a guide is selected that directs the CRISPR-Cas system to make a deletion in the JUNB gene such that a frame-shift is introduced into the ORF causing a decrease in expression of the full-length JUNB transcript. In one example embodiment, a guide sequence is selected that directs the CRISPR-Cas system to make one or more insertions that affects the splicing site in the JUNB gene, which causes a decrease in the expression of the JUNB gene. In an embodiment, a guide is selected that directs the CRISPR-Cas system to make one or more insertions in the JUNB gene such that a frame-shift is introduced into the ORF causing a decrease in expression of the full-length JUNB transcript.

In one example embodiment, a guide is selected that directs the CRISPR-Cas system to make one or more deletions that affects or disrupts one or more phosphorylation sites and/or the acetylation site, or combinations thereof, which cause protein instability and a decrease in the activity of the JUNB protein. In an embodiment, a guide is selected that directs the CRISPR-Cas system to make a deletion in the JUNB gene such that a frame-shift is introduced into the ORF introducing a stop codon which leads to a decrease in activity of the truncated JUNB protein. In one example embodiment, a guide is selected that directs the CRISPR-Cas system to make one or more deletions or insertions of the JUNB gene which leads to disruption of the polyA signal sequence region of the JUNB protein, causing a decrease in activity of the JUNB protein.

### HDR Template Based Editing

In one example embodiment, a donor template is provided to replace a genomic sequence comprising one or more variants that reduce B3GNT2 expression. A donor template may comprise an insertion sequence flanked by two homology regions. The insertion sequence comprises an edited sequence to be inserted in place of the target sequence (e.g. a portion of genomic DNA comprising the one or more variants). The homology regions comprise sequences that are homologous to the genomic DNA strands at the site of the CRISPR-Cas induced double-strand break. Cellular HDR mechanisms then facilitate insertion of the insertion sequence at the site of the DSB. Accordingly, in certain example embodiments, a donor template and guide sequence are selected to direct excision and replacement of a section of genome DNA comprising a variant that reduces B3GNT2 expression with an insertion sequence that edits the one or more variants to a wild-type or non-risk variant. In one example embodiment, the insertion sequence comprises a wild-type or non-risk variant that reduces B3GNT2 expression. In one example embodiment, the insertion sequence encodes a portion of genomic DNA in which the variant is changed from a C to a T.

The donor template may include a sequence which results in a change in sequence of 1, 2, 3, 4, 5, 6, 7, 8, 9, 10, 11, 12 or more nucleotides of the target sequence.

A donor template may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 1 10+/−10, 120+/−10, 130+/−10, 140+/−10, 150+/−10, 160+/−10, 170+/−10, 1 80+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 1 10+/−20, 120+/−20, 130+/−20, 140+/−20, I 50+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to300, 50 to 200, or 50 to 100 nucleotides in length.

The homology regions of the donor template may be complementary to a portion of a polynucleotide comprising the target sequence. When optimally aligned, a donor template might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The donor template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

Homology arms of the donor template may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

In one example embodiment, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

The donor template may further comprise a marker. Such a marker may make it easy to screen for targeted integrations. Examples of suitable markers include restriction sites, fluorescent proteins, or selectable markers. The donor template of the disclosure can be constructed using recombinant techniques (see, for example, Sambrook et al., 2001 and Ausubel et al., 1996).

In one example embodiment, a donor template is a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

### Class 1 Systems

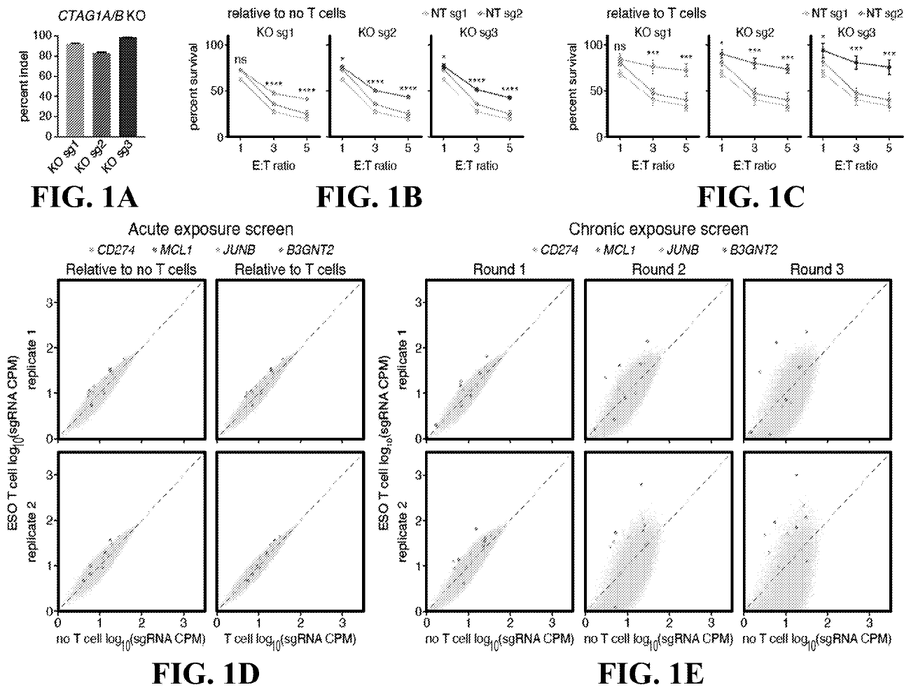

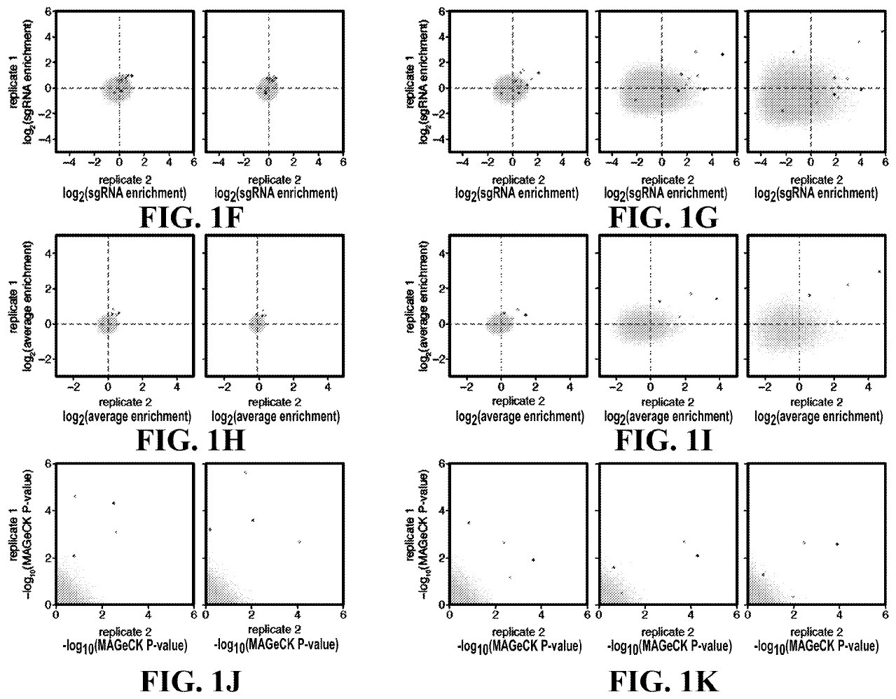

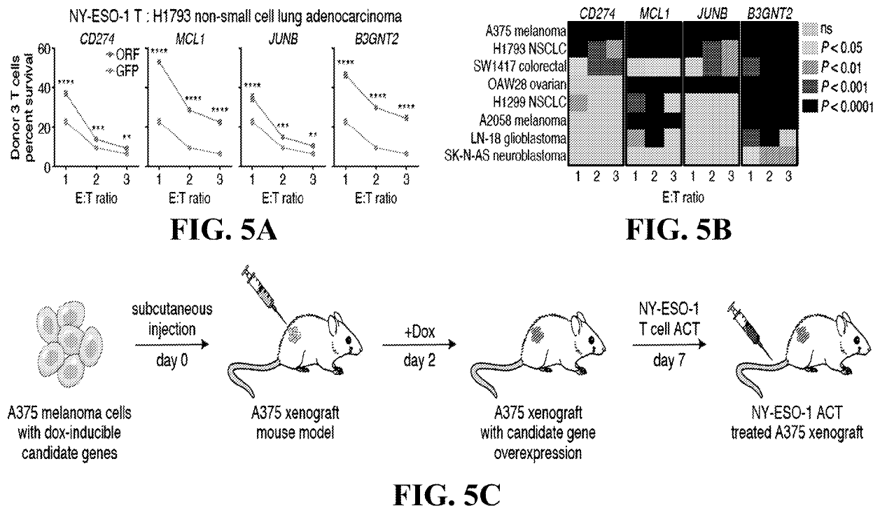

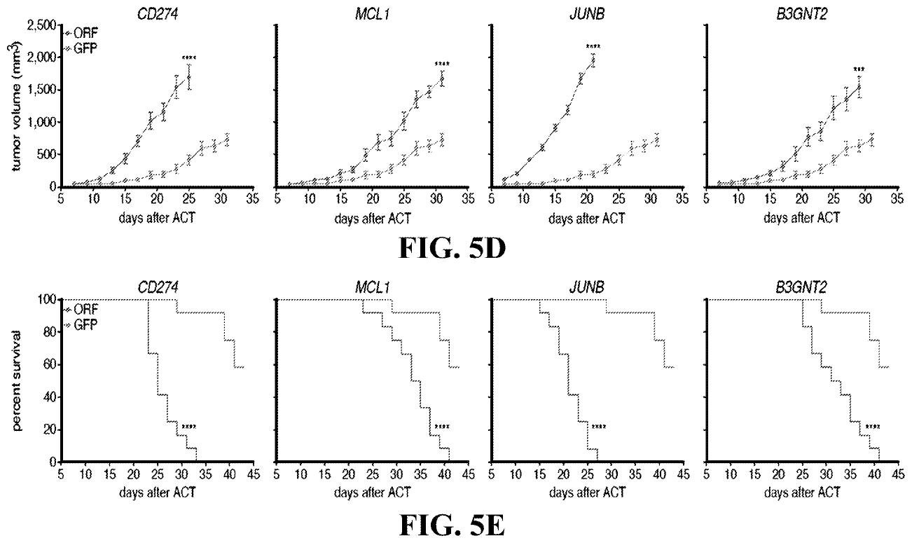

The CRISPR-Cas therapeutic methods disclosed herein may be designed for use with Class 1 CRISPR-Cas systems. In certain example embodiments, the Class 1 system may be Type I, Type III or Type IV CRISPR-Cas as described in Makarova et al. “Evolutionary classification of CRISPR-Cas systems: a burst of class 2 and derived variants” Nature Reviews Microbiology, 18:67-81 (February 2020), incorporated in its entirety herein by reference, and particularly as described in FIG. 1, p. 326. The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g. Cas1, Cas2, RNA nuclease), and/or one or more accessory proteins (e.g. Cas 4, DNA nuclease), CRISPR associated Rossman fold (CARF) domain containing proteins, and/or RNA transcriptase. Although Class 1 systems have limited sequence similarity, Class 1 system proteins can be identified by their similar architectures, including one or more Repeat Associated Mysterious Protein (RAMP) family subunits, e.g. Cas 5, Cas6, Cas7. RAMP proteins are characterized by having one or more RNA recognition motif domains. Large subunits (for example cas8 or cas10) and small subunits (for example, cas11) are also typical of Class 1 systems. See, e.g., FIGS. 1 and 2. Koonin E V, Makarova K S. 2019 Origins and evolution of CRISPR-Cas systems. Phil. Trans. R. Soc. B 374: 20180087, DOI: 10.1098/rstb.2018.0087. In one aspect, Class 1 systems are characterized by the signature protein Cas3. The Cascade in particular Class1 proteins can comprise a dedicated complex of multiple Cas proteins that binds pre-crRNA and recruits an additional Cas protein, for example Cas6 or Cas5, which is the nuclease directly responsible for processing pre-crRNA. In one aspect, the Type I CRISPR protein comprises an effector complex comprises one or more Cas5 subunits and two or more Cas7 subunits. Class 1 subtypes include Type I-A, I-B, I-C, I-U, I-D, I-E, and I-F, Type IV-A and IV-B, and Type III-A, III-D, III-C, and III-B. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas. 1709035114; see also, Makarova et al, the CRISPR Journal, v. 1, n5, FIG. 5.

### Class 2 Systems

The CRISPR-Cas therapeutic methods disclosed herein may be designed for use with. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside a split Ruv-C like nuclease domain sequence. The Type V systems (e.g., Cas12) only contain a RuvC-like nuclease domain that cleaves both strands. Some Type V systems have also been found to possess this collateral activity with two single-stranded DNA in in vitro contexts.

In one example embodiment, the Class 2 system is a Type II system. In one example embodiment, the Type II CRISPR-Cas system is a II-A CRISPR-Cas system. In one example embodiment, the Type II CRISPR-Cas system is a II-B CRISPR-Cas system. In one example embodiment, the Type II CRISPR-Cas system is a II-C1 CRISPR-Cas system. In one example embodiment, the Type II CRISPR-Cas system is a II-C2 CRISPR-Cas system. In one example embodiments, the Type II system is a Cas9 system. In some embodiments, the Type II system includes a Cas9.

In one example embodiment, the Class 2 system is a Type V system. In one example embodiment, the Type V CRISPR-Cas system is a V-A CRISPR-Cas system. In one example embodiment, the Type V CRISPR-Cas system is a V-B1 CRISPR-Cas system. In one example embodiment, the Type V CRISPR-Cas system is a V-B2 CRISPR-Cas system. In one example embodiment, the Type V CRISPR-Cas system is a V-C CRISPR-Cas system. In one example embodiment, the Type V CRISPR-Cas system is a V-D CRISPR-Cas system. In one example embodiment, the Type V CRISPR-Cas system is a V-E CRISPR-Cas system. In one example embodiment, the Type V CRISPR-Cas system is a V-F1 CRISPR-Cas system. In one example embodiment, the Type V CRISPR-Cas system is a V-F1 (V-U3) CRISPR-Cas system. In one example embodiment, the Type V CRISPR-Cas system is a V-F2 CRISPR-Cas system. In one example embodiment, the Type V CRISPR-Cas system is a V-F3 CRISPR-Cas system. In one example embodiment, the Type V CRISPR-Cas system is a V-G CRISPR-Cas system. In one example embodiment, the Type V CRISPR-Cas system is a V-H CRISPR-Cas system. In one example embodiment, the Type V CRISPR-Cas system is a V-I CRISPR-Cas system. In one example embodiment, the Type V CRISPR-Cas system is a V-K (V-U5) CRISPR-Cas system. In one example embodiment, the Type V CRISPR-Cas system is a V-U1 CRISPR-Cas system. In one example embodiment, the Type V CRISPR-Cas system is a V-U2 CRISPR-Cas system. In one example embodiment, the Type V CRISPR-Cas system is a V-U4 CRISPR-Cas system. In one example embodiment, the Type V CRISPR-Cas is a Cas12a (Cpf1), Cas12b (C2c1), Cas12c (C2c3), Cas12d (CasY), Cas12e (CasX), Cas14, and/or CasΦ.

### Guide Molecules

The following include general design principles that may be applied to the guide molecule. The terms guide molecule, guide sequence and guide polynucleotide refer to polynucleotides capable of guiding Cas to a target genomic locus and are used interchangeably as in foregoing cited documents such as International Patent Publication No. WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide sequence, and hence a nucleic acid-targeting guide, may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In one example embodiment, a guide RNA or crRNA may comprise, consist essentially of, or consist of a direct repeat (DR) sequence and a guide sequence or spacer sequence. In another example embodiment, the guide RNA or crRNA may comprise, consist essentially of, or consist of a direct repeat sequence fused or linked to a guide sequence or spacer sequence. In another example embodiment, the direct repeat sequence may be located upstream (i.e., 5′) from the guide sequence or spacer sequence. In other embodiments, the direct repeat sequence may be located downstream (i.e., 3′) from the guide sequence or spacer sequence.

In one example embodiment, the crRNA comprises a stem loop, preferably a single stem loop. In one example embodiment, the direct repeat sequence forms a stem loop, preferably a single stem loop.

In one example embodiment, the spacer length of the guide RNA is from 15 to 35 nt. In another example embodiment, the spacer length of the guide RNA is at least 15 nucleotides. In another example embodiment, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it being advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All of (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in International Patent Application No. PCT US2019/045582, specifically paragraphs [0178]-[0333]. which is incorporated herein by reference.

### Target Sequences, PAMs, and PESs

In the context of formation of a CRISPR complex, “target sequence” refers to a sequence to which a guide sequence is designed to have complementarity, where hybridization between a target sequence and a guide sequence promotes the formation of a CRISPR complex. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

PAM elements are sequences that can be recognized and bound by Cas proteins. Cas proteins/effector complexes can then unwind the dsDNA at a position adjacent to the PAM element. It will be appreciated that Cas proteins and systems target RNA do not require PAM sequences (Marraffini et al. 2010. Nature. 463:568-571). Instead, many rely on PFSs, which are discussed elsewhere herein. In one example embodiment, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 3 (from Gleditzsch et al. 2019) below shows several Cas polypeptides and the PAM sequence they recognize.

TABLE 3
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a (Cpf1) | TTTV |
| (including | |
| LbCpf1 and | |
| AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |
| Cas1 | 5′-CTT-3′ |
| Cas8e | 5′-ATG-3′ |
| Type I-A | 5′-CCN-3′ |
| Type I-B | TTC, ACT, TAA, TAT, |
| | TAG, and CAC |
| Type I-C | NTTC |
| Type I-E | 5′-AAG-3′ |
| Type I-F | GG |

In a preferred embodiment, the CRISPR effector protein may recognize a 3′ PAM. In one example embodiment, the CRISPR effector protein may recognize a 3′ PAM which is 5′H, wherein His A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas 13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead, such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Sequences Related to Nucleus Targeting and Transportation

In some embodiments, one or more components (e.g., the Cas protein) in the composition for engineering cells may comprise one or more sequences related to nucleus targeting and transportation. Such sequences may facilitate the one or more components in the composition for targeting a sequence within a cell. In order to improve targeting of the CRISPR-Cas protein used in the methods of the present disclosure to the nucleus, it may be advantageous to provide one or both of these components with one or more nuclear localization sequences (NLSs).

In one example embodiment, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID NO: 1) or PKKKRKVEAS (SEQ ID NO: 2); the NLS from nucleoplasmin (e.g., the nucleoplasmin bipartite NLS with the sequence KRPAATKKAGQAKKKK (SEQ ID NO: 3)); the c-myc NLS having the amino acid sequence PAAKRVKLD (SEQ ID NO: 4) or RQRRNELKRSP (SEQ ID NO: 5); the hRNPA1 M9 NLS having the sequence NQSSNFGPMKGGNFGGRSSGPYGGGGQYFAKPRNQGGY (SEQ ID NO: 6); the sequence RMRIZFKNKGKDTAELRRRRVEVSVELRKAKKDEQILKRRNV (SEQ ID NO: 7) of the IBB domain from importin-alpha; the sequences VSRKRPRP (SEQ ID NO: 8) and PPKKARED (SEQ ID NO: 9) of the myoma T protein; the sequence PQPKKKPL (SEQ ID NO: 10) of human p53; the sequence SALIKKKKKMAP (SEQ ID NO: 11) of mouse c-abl IV; the sequences DRLRR (SEQ ID NO: 12) and PKQKKRK (SEQ ID NO: 13) of the influenza virus NS1; the sequence RKLKKKIKKL (SEQ ID NO: 14) of the Hepatitis virus delta antigen; the sequence REKKKFLKRR (SEQ ID NO: 15) of the mouse Mx1 protein; the sequence KRKGDEVDGVDEVAKKKSKK (SEQ ID NO: 16) of the human poly(ADP-ribose) polymerase; and the sequence RKCLQAGMNLEARKTKK (SEQ ID NO: 17) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the Cas protein, or exposed to a Cas protein lacking the one or more NLSs.

The Cas proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the Cas proteins, an NLS attached to the C-terminal of the protein.

### Zinc-Finger Nucleases (ZFN)

Other preferred tools for genome editing for use in the context of this invention include zinc finger systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP). Zinc-finger nuclease (ZFNs) have been used to modify endogenous genes in various organisms, including viruses, bacteria, nematodes, frogs, plants, insects, fish and mammals such as mice, rats and pigs, as well as in cultured mammalian and avian cells.

In one example embodiment, a method of enhancing the anti-tumor immunity of a subject in need thereof comprises administering administering a zinc-finger nuclease editing system that generates variants with decreased expression or activity of B3GNT2.

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### TALENs

As disclosed herein editing can be made by way of the transcription activator-like effector nucleases (TALENs) system. In some embodiments, the programmable nuclease may be a transcription activator-like effector nuclease (TALEN), a functional fragment thereof, or a variant thereof. The present disclosure also includes nucleotide sequences that are or encode one or more components of a TALEN system. As disclosed herein, editing can be made by way of the transcription activator-like effector (TALEs) system, which have been used to modify endogenous genes in various species, including viruses, yeast, plants, nematodes, insects, frogs, fish and mammals such as mice, rats and pigs, as well as in cultured mammalian cells.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference. The structure and function of TALEs is further described in, for example, Moscou et al., Science 326:1501 (2009); Boch et al., Science 326:1509-1512 (2009); and Zhang et al., Nature Biotechnology 29:149-153 (2011).

In some embodiments, provided herein include isolated, non-naturally occurring, recombinant or engineered DNA binding proteins that comprise TALE monomers as a part of their organizational structure that enable the targeting of nucleic acid sequences with improved efficiency and expanded specificity.

In one example embodiment, a method of enhancing the anti-tumor immunity of a subject in need thereof comprises administering administering a TALENs editing system that generates variants with decreased expression or activity of B3GNT2.

In some embodiments, TALENs can be designed to target almost any given DNA sequence, which is a crucial advantage of TALENs over other types of nucleases. For example, small DNA sequences (such as enhancers or miRNA-coding sequences) may lack targetable sites for ZFNs or CRISPR-Cas systems but can be mutated preferentially using TALENs. The only limitation in the design of TALENs seems to be the requirement for a thymine at the 5′ end of the target sequence, which is recognized by two amino-terminal cryptic repeat folds. Although there have been conflicting reports that emphasize or refute the importance of this 5′ thymine, choosing a target sequence with a thymine at the 5′ end is usually recommended. Recently developed TALE variants that recognize other bases at the 5′ end would further broaden the range of targetable sites. Conventional TALENs cannot cleave target DNA that contains methylated cytosines. However, a methylated cytosine is indistinguishable from a thymine in the major groove; hence, the His-Asp RVD repeat (which recognizes cytosines) can be replaced with an Asn-Gly RVD repeat (which recognizes thymines) to generate TALENs that can cleave methylated DNA.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

### Meganucleases

In some embodiments, the programmable nuclease may be a meganuclease or system thereof. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated by reference.

In one example embodiment, a method of enhancing the anti-tumor immunity of a subject in need thereof comprises administering administering a meganuclease or system thereof that generates variants with decreased expression or activity of B3GNT2.

### Base Editing

Provided herein are methods for editing DNA or RNA, i.e., base editing, without inducing double-stranded breaks in the DNA targeted for modification. A base-editing system may comprise a Cas polypeptide linked to a nucleobase deaminase (“base editing system”) and a guide molecule capable of forming a complex with the Cas polypeptide and directing sequence-specific binding of the base editing system at a target sequence. In one example embodiment, the Cas polypeptide is catalytically inactive. In another example embodiment, the Cas polypeptide is a nickase. The Cas polypeptide may be any of the Cas polypeptides disclosed above. In one example embodiment, the Cas polypeptide is a Type II Cas polypeptide. In one example embodiment, the Cas polypeptide is a Cas9 polypeptide. In another example embodiment, the Cas polypeptide is a Type V Cas polypeptide. In one example embodiment, the Cas polypeptide is a Cas 12a or Cas 12b polypeptide. The nucleobase deaminase may be cytosine base editor (CBE) or adenosine base editors (ABEs). CBEs convert C·G base pairs into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). Example base editing systems are disclosed in Rees and Liu (2018), Nat. Rev. Genet. 19(12): 770-788, particularly at FIGS. 1b, 2a-2c, 3a-3f, and Table 1, which is specifically incorporated herein by reference. In certain example embodiments, the base editing system may further comprise a DNA glycosylase inhibitor.

In one example embodiment, a method of enhancing the anti-tumor immunity of a subject in need thereof comprises administering a base editing system that generates one or more variants with decreased expression or activity of B3GNT2.

The editing window of a base editing system may range over a 5-8 nucleotide window, depending on the base editing system used. Id. Accordingly, given the base editing system used, a guide sequence may be selected to direct the base editing system to convert a base or base pair of one or more variants resulting in reduced B3GNT2 expression. In one example embodiment, the variant is generated as using one or more of the gene editing methods described herein.

### ARCUS Based Editing

In one example embodiment, a method of enhancing the anti-tumor immunity of a subject in need thereof comprises administering administering an ARCUS base editing system. Exemplary methods for using ARCUS can be found in U.S. Pat. No. 10,851,358, US Publication No. 2020-0239544, and WIPO Publication No. 2020/206231 which are incorporated herein by reference

### DNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein, “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In some embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C·G base pair into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12): 770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471. Base editors may be further engineered to optimize conversion of nucleotides (e.g. A:T to G:C). Richter et al. 2020. Nature Biotechnology. doi.org/10.1038/s41587-020-0453-z.

Other Example Type V base editing systems are described in International Patent Publication Nos. WO 2018/213708, WO 2018/213726, and International Patent Applications No. PCT/US2018/067207, PCT/US2018/067225, and PCT/US2018/067307, each of which is incorporated herein by reference.

### RNA Base Editing

The present application relates, in part, to modifying a target RNA sequence of interest. In certain example embodiments, the base editing system may be a RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA based editor may be used to delete or introduce a post-translational modification site in the expressed mRNA. In contrast to DNA base editors, whose edits are permanent in the modified cell, RNA base editors can provide edits where finer temporal control may be needed, for example in modulating a particular immune response. Example Type VI RNA-base editing systems are described in Cox et al. 2017. Science 358: 1019-1027, WO 2019/005884, WO 2019/005886, WO 2019/071048, PCT/US20018/05179, PCT/US2018/067207, which are incorporated herein by reference. An example FnCas9 system that may be adapted for RNA base editing purposes is described in WO 2016/106236, which is incorporated herein by reference.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

Using RNA-targeting rather than DNA targeting offers several advantages relevant for therapeutic development. First, there are substantial safety benefits to targeting RNA: there will be fewer off-target events because the available sequence space in the transcriptome is significantly smaller than the genome, and if an off-target event does occur, it will be transient and less likely to induce negative side effects. Second, RNA-targeting therapeutics will be more efficient because they are cell-type independent and do not have to enter the nucleus, making them easier to deliver.

In an embodiment, the present disclosure includes an engineered composition for site-directed base editing comprising: a targeting domain; and an adenosine deaminase or catalytic domain thereof, wherein the adenosine deaminase is modified to convert activity to a cytidine deaminase.

In some embodiments, the adenosine deaminase is modified by one or more mutations at one or more positions selected from E396, C451, V351, R455, T375, K376, S486, Q488, R510, K594, R348, G593, S397, H443, L444, Y445, F442, E438, T448, A353, V355, T339, P539, V525 and 1520. In some embodiments, the adenosine deaminase is mutated at one or more positions selected from E488, V351, S486, T375, S370, P462, and N597. In some embodiments, the adenosine deaminase comprises one or more mutations selected from E488Q, V351G, S486A, T375S, S370C, P462A, and N597I. In some embodiments, the adenosine deaminase protein or catalytic domain thereof is a human, cephalopod, or Drosophila adenosine deaminase protein or catalytic domain thereof. In some embodiments, said adenosine deaminase protein or catalytic domain thereof has been modified to comprise a mutation at glutamic acid488 of the hADAR2-D amino acid sequence, or a corresponding position in a homologous ADAR protein. In some embodiments, said glutamic acid residue at position 488 or a corresponding position in a homologous ADAR protein is replaced by a glutamine residue (E488Q). In some embodiments, said adenosine deaminase protein or catalytic domain thereof is a mutated hADAR2d comprising mutation E488Q or a mutated hADAR1d comprising mutation E1008Q. In some embodiments, the targeting domain is a catalytically inactive Cas13 protein, or a nucleotide sequence encoding said catalytically inactive Cas13 protein. In some embodiments, the catalytically inactive Cas13 protein is catalytically inactive Cas13a, catalytically inactive Cas 13b, or catalytically inactive Cas13c. In some embodiments, said catalytically inactive Cas13 protein is obtained from a Cas13 nuclease derived from a bacterial species selected from the group consisting of the bacterial species listed in any of Tables 1, 2, 3, or 4. In some embodiments, the composition further comprising a guide molecule which comprises a guide sequence linked to a direct repeat sequence, or a nucleotide sequence encoding said guide molecule. In some embodiments, said adenosine deaminase protein or catalytic domain thereof is covalently or non-covalently linked to the targeting domain.

In another embodiment, the disclosure relates to a method of modifying an Adenine in a target RNA sequence of interest. In particular embodiments, the method comprises delivering to said target RNA: (a) a catalytically inactive (dead) Cas13 protein; (b) a guide molecule which comprises a guide sequence linked to a direct repeat sequence; and (c) an adenosine deaminase protein or catalytic domain thereof; wherein said adenosine deaminase protein or catalytic domain thereof is covalently or non-covalently linked to said dead Cas 13 protein or said guide molecule or is adapted to link thereto after delivery; wherein guide molecule forms a complex with said dead Cas13 protein and directs said complex to bind said target RNA sequence of interest, wherein said guide sequence is capable of hybridizing with a target sequence comprising said Adenine to form an RNA duplex, wherein said guide sequence comprises a non-pairing Cytosine at a position corresponding to said Adenine resulting in an A-C mismatch in the RNA duplex formed; wherein said adenosine deaminase protein or catalytic domain thereof deaminates said Adenine in said RNA duplex.

In some embodiments, the RNA editing is carried out using the Cas13 protein, wherein the Cas13 protein is Cas13a, Cas13b or Cas13c.

The adenosine deaminase protein or catalytic domain thereof is fused to N- or C-terminus of said dead Cas13 protein. In some embodiments, the adenosine deaminase protein or catalytic domain thereof is fused to said dead Cas13 protein by a linker.

In some embodiments, the adenosine deaminase protein or catalytic domain thereof is linked to an adaptor protein and said guide molecule or said dead Cas 13 protein comprises an aptamer sequence capable of binding to said adaptor protein. The adaptor sequence may be selected from MS2, PP7, QB, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s and PRR1.

In some embodiments, the adenosine deaminase protein or catalytic domain thereof is inserted into an internal loop of said dead Cas13 protein. In some embodiments, the Cas13a protein comprises one or more mutations in the two HEPN domains, particularly at position R474 and R1046 of Cas 13a protein originating from Leptotrichia wadei or amino acid positions corresponding thereto of a Cas13a ortholog.

In some embodiments, the Cas13 protein is a Cas13b proteins, and the Cas13b comprises a mutation in one or more of positions R116, H121, R1177, H1182 of Cas13b protein originating from Bergeyella zoohelcum ATCC 43767 or amino acid positions corresponding thereto of a Cas 13b ortholog. In some embodiments, the mutation is one or more of R116A, H121A, R1177A, H1182A of Cas13b protein originating from Bergeyella zoohelcum ATCC 43767 or amino acid positions corresponding thereto of a Cas13b ortholog.

In some embodiments, the guide sequence has a length of about 29-53 nt capable of forming said RNA duplex with said target sequence. In some embodiments, the guide sequence has a length of about 40-50 nt capable of forming said RNA duplex with said target sequence. In some embodiments, the distance between said non-pairing C and the 5′ end of said guide sequence is 20-30 nucleotides.

In some embodiments, the adenosine deaminase protein or catalytic domain thereof is a human, cephalopod, or Drosophila adenosine deaminase protein or catalytic domain thereof. In certain example embodiments, the adenosine deaminase protein or catalytic domain thereof has been modified to comprise a mutation at glutamic acid488 of the hADAR2-D amino acid sequence, or a corresponding position in a homologous ADAR protein. In some embodiments, the glutamic acid residue may be at position 488 or a corresponding position in a homologous ADAR protein is replaced by a glutamine residue (E488Q).

In some embodiments, the adenosine deaminase protein or catalytic domain thereof is a mutated hADAR2d comprising mutation E488Q or a mutated hADAR1d comprising mutation E1008Q.

In some embodiments, the guide sequence comprises more than one mismatch corresponding to different adenosine sites in the target RNA sequence or wherein two guide molecules are used, each comprising a mismatch corresponding to a different adenosine site in the target RNA sequence.

In some embodiments, the Cas13 protein and optionally said adenosine deaminase protein or catalytic domain thereof comprise one or more heterologous nuclear localization signal(s) (NLS(s)).

In some embodiments, the method further comprises, determining the target sequence of interest and selecting an adenosine deaminase protein or catalytic domain thereof which most efficiently deaminates said adenine present in then target sequence.

The components of the systems described herein may be delivered to said cell as a ribonucleoprotein complex or as one or more polynucleotide molecules. The one or more polynucleotide molecules may comprise one or more mRNA molecules encoding the components. The one or more polynucleotide molecules may be comprised within one or more vectors. The one or more polynucleotide molecules may further comprise one or more regulatory elements operably configured to express said Cas13 protein, said guide molecule, and said adenosine deaminase protein or catalytic domain thereof, optionally wherein said one or more regulatory elements comprise inducible promoters. The one or more polynucleotide molecules or said ribonucleoprotein complex may be delivered via particles, vesicles, or one or more viral vectors. The particles may comprise a lipid, a sugar, a metal or a protein. The particles may comprise lipid nanoparticles. The vesicles may comprise exosomes or liposomes. The one or more viral vectors may comprise one or more of adenovirus, one or more lentivirus or one or more adeno-associated virus.

The RNA editing methods disclosed herein may be used to modify a cell, a cell line or an organism by manipulation of one or more target RNA sequences.

In some embodiments, the deamination of said Adenine in said target RNA of interest remedies a disease caused by transcripts containing a pathogenic G→A or C→T point mutation.

The methods disclosed herein, may be used to make a modification that affects specific, targeted genes of an organism (e.g., B3GNT2). The modification may affect splicing of said target RNA sequence. The modification may introduce a mutation in a transcript that reduces expression of the targeted gene. The modification may introduce an amino acid change and cause a reduction in activity of the targeted protein.

In some embodiments, the deamination of the adenine in said target RNA of interest causes a loss of function or reduced expression of a gene. In certain example embodiments, the loss of function or reduced expression of the gene leads to an enhancement of anti-tumor immunity in a subject.

In some embodiments, the cytosine of the adenosine deaminase is not 5′ flanked by guanosine. In certain embodiments, said adenosine deaminase is ADAR, optionally huADAR, optionally (hu)ADAR1 or (hu)ADAR2. In certain embodiments, said Cas13, preferably Cas13b, is truncated, preferably C-terminally truncated, preferably wherein said Cas 13 is a truncated functional variant of the corresponding wild type Cas13.

In another aspect, the present disclosure includes a method of modifying an Adenine in a target RNA sequence of interest, comprising delivering to said target RNA: (a) a catalytically inactive (dead) Cas13 protein; (b) a guide molecule which comprises a guide sequence linked to a direct repeat sequence; and (c) an adenosine deaminase protein or catalytic domain thereof mutated to convert activity to a cytidine deaminase; wherein said adenosine deaminase protein or catalytic domain thereof is covalently or non-covalently linked to said dead Cas13 protein or said guide molecule or is adapted to link thereto after delivery; wherein said guide molecule forms a complex with said dead Cas13 protein and directs said complex to bind said target RNA sequence of interest, wherein said guide sequence is capable of hybridizing with a target sequence comprising said adenine to form an RNA duplex, wherein said guide sequence comprises a non-pairing cytosine at a position corresponding to said adenine resulting in an A-C mismatch in the RNA duplex formed; wherein said adenosine deaminase protein or catalytic domain thereof deaminates said adenine in said RNA duplex.

In some embodiments, the adenosine deaminase is mutated at one or more positions selected from E396, C451, V351, R455, T375, K376, S486, Q488, R510, K594, R348, G593, S397, H443, L444, Y445, F442, E438, T448, A353, V355, T339, P539, V525 and I520. In some example embodiments, the adenosine deaminase is mutated at one or more positions selected from E488, V351, S486, T375, S370, P462, and N597.

In some embodiments, the present disclosure includes an engineered, non-naturally occurring RNA editing system suitable for modifying an adenine in a target locus of interest. comprising (a) a guide molecule which comprises a guide sequence linked to a direct repeat sequence, or a nucleotide sequence encoding said guide molecule; (b) a catalytically inactive Cas13 protein, or a nucleotide sequence encoding said catalytically inactive Cas13 protein; (c) an adenosine deaminase protein or catalytic domain thereof, or a nucleotide sequence encoding said adenosine deaminase protein or catalytic domain thereof; wherein the adenosine deaminase is modified to convert activity to a cytidine deaminase; wherein said adenosine deaminase protein or catalytic domain thereof is covalently or non-covalently linked to said Cas13 protein or said guide molecule or is adapted to link thereto after delivery; wherein said guide sequence is capable of hybridizing with a target RNA sequence comprising an adenine to form an RNA duplex, wherein said guide sequence comprises a non-pairing cytosine at a position corresponding to said Adenine resulting in an A-C mismatch in the RNA duplex formed

In some embodiments, the adenosine deaminase is modified by one or more mutations selected from E396, C451, V351, R455, T375, K376, S486, Q488, R510, K594, R348, G593, S397, H443, L444, Y445, F442, E438, T448, A353, V355, T339, P539, V525 and I520. In some embodiments, the adenosine deaminase is mutated at one or more positions selected from E488, V351, S486, T375, S370, P462, and N597.

### Prime Editing

In one example embodiment, a method of enhancing the anti-tumor immunity of a subject in need thereof comprises administering administering a prime editing system that generates one or more variants with decreased expression or activity of B3GNT21. Like base editing systems, prime editing systems are capable of targeted modification of a polynucleotides without generating double stranded breaks. See e.g. Anzalone et al. 2019. Nature. 576: 149-157, incorporated herein by reference. Prime editing can operate via a “search-and-replace” methodology and can mediate targeted insertions, deletions, as well as all 12 possible base-to-base conversion and combinations thereof.

In one example embodiment, a prime editing system comprises a Cas polypeptide having nickase activity, a reverse transcriptase, and a prime editing guide RNA (pegRNA). Cas polypeptide, and/or reverse transcriptase can be coupled together or otherwise associate with each other to form a prime editing complex and edit a target sequence. The Cas polypeptide may be any of the Cas polypeptides disclosed above. In one example embodiment, the Cas polypeptide is a Type II Cas polypeptide. In another example embodiment, the Cas polypeptide is a Cas9 nickase. In one example embodiment, the Cas polypeptide is a Type V Cas polypeptide. In another example embodiment, the Cas polypeptide is a Cas12a or Cas12b.

The prime editing guide molecule (pegRNA) comprises a primer binding site (PBS) configured to hybridize with a portion of a nicked strand on a target polynucleotide (e.g. genomic DNA) a reverse transcriptase (RT) template comprising the edit to be inserted in the genomic DNA and a spacer sequence designed to hybridize to a target sequence at the site of the desired edit. The nicking site is dependent on the Cas polypeptide used and standard cutting preference for that Cas polypeptide relative to the PAM. Thus, based on the Cas polypeptide used, a pegRNA can be designed to direct the prime editing system to introduce a nick where the desired edit should take place. In one example embodiment, a pegRNA is configured to direct the prime editing system to convert a single base or base pair of the one or more variants associated with reduced B3GNT2 expression. In one example embodiment, a pegRNA is configured to direct the prime editing system to convert a single base or base pair of one or more variants associated with reduced B3GNT2 expression such that B3GNT2 protein activity is reduced.

The pegRNA can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c

### CRISPR-Associated Transposases (CAST) Systems

In one example embodiment, a method of enhancing the anti-tumor immunity of a subject in need thereof comprises administering a CAST system that incorporates or inserts a genomic region comprising one or more variants associated with decreased expression or activity of B3GNT2. In one example embodiment, a CAST system is used to replace all or a portion of B3GNT2 comprising one or more variants that reduce B3GNT2 expression or activity. In one example embodiment, the variant is generated using one or more of the gene editing methods described herein.

CAST systems comprise a Cas polypeptide, a guide sequence, a transposase, and a donor construct. The transposase is linked to or otherwise capable of forming a complex with the Cas polypeptide. The donor construct comprises a donor sequence to be inserted into a target polynucleotide and one or more transposase recognition elements. The transposase is capable of binding the donor construct and excising the donor template and directing insertion of the donor template into a target site on a target polynucleotide (e.g. genomic DNA). The guide molecule is capable of forming a CRISPR-Cas complex with the Cas polypeptide, and can be programmed to direct the entire CAST complex such that the transposase is positioned to insert the donor sequence at the target site on the target polynucleotide. For multimeric transposase, only those transposases needed for recognition of the donor construct and transposition of the donor sequence into the target polypeptide may be required. The Cas may be naturally catalytically inactive or engineered to be catalyically inactive.

In one example embodiment, the CAST system is a Tn7-like CAST system, wherein the transposase comprises one or more polypeptides from a Tn7 or Tn7-like transposase. The Cas polypeptide of the Tn7-like transposase may be a Class 1 (multimeric effector complex) or Class 2 (single protein effector) Cas polypeptide.

In one example embodiments, the Cas polypeptide is a Class 1 Type-If Cas polypeptide. In one example embodiment, the Cas polypeptide may comprise a cas6, a cas7, and a cas8-cas5 fusion. In one example embodiments, the Tn7 transposase may comprise TnsB, TnsC, and TniQ. In another example embodiment, the Tn7 transposase may comprise TnsB, TnsC, and TnsD. In certain example embodiments, the Tn7 transposase may comprise TnsD, TnsE, or both. As used herein, the terms “TnsAB”, “TnsAC”, “TnsBC”, or “TnsABC” refer to a transponson complex comprising TnsA and TnsB, TnsA and TnsC, TnsB and TnsC, TnsA and TnsB and TnsC, respectively. In these combinations, the transposases (TnsA, TnsB, TnsC) may form complexes or fusion proteins with each other. Similarly, the term TnsABC-TniQ refer to a transposon comprising TnsA, TnsB, TnsC, and TniQ, in a form of complex or fusion protein. An example Type If-Tn7 CAST system is described in Klompe et al. Nature, 2019, 571:219-224 and Vo et al. bioRxiv, 2021, doi.org/10.1101/2021.02.11.430876, which are incorporated herein by reference.

In one example embodiment, the Cas polypeptide is a Class 1 Type-1b Cas polypeptide. In one example embodiment, the Cas polypeptide may comprise a cas6, a cas7, and a cas8b (e.g. a ca8b3). In one example embodiments, the Tn7 transposase may comprise TnsB, TnsC, and TniQ. In another example embodiment, the Tn7 transposase may comprise TnsB, TnsC, and TnsD. In certain example embodiments, the Tn7 transposase may comprise TnsD, TnsE, or both. As used herein, the terms “TnsAB”, “TnsAC”, “TnsBC”, or “TnsABC” refer to a transponson complex comprising TnsA and TnsB, TnsA and TnsC, TnsB and TnsC, TnsA and TnsB and TnsC, respectively. In these combinations, the transposases (TnsA, TnsB, TnsC) may form complexes or fusion proteins with each other. Similarly, the term TnsABC-TniQ refer to a transposon comprising TnsA, TnsB, TnsC, and TniQ, in a form of complex or fusion protein.

In one example embodiment, the Cas polypeptide is Class 2, Type V Cas polypeptide. In one example embodiment, the Type V Cas polypeptide is a Cas12k. In one example embodiments, the Tn7 transposase may comprise TnsB, TnsC, and TniQ. In another example embodiment, the Tn7 transposase may comprise TnsB, TnsC, and TnsD. In certain example embodiments, the Tn7 transposase may comprise TnsD, TnsE, or both. As used herein, the terms “TnsAB”, “TnsAC”, “TnsBC”, or “TnsABC” refer to a transponson complex comprising TnsA and TnsB, TnsA and TnsC, TnsB and TnsC, TnsA and TnsB and TnsC, respectively. In these combinations, the transposases (TnsA, TnsB, TnsC) may form complexes or fusion proteins with each other. Similarly, the term TnsABC-TniQ refer to a transposon comprising TnsA, TnsB, TnsC, and TniQ, in a form of complex or fusion protein. An example Cas12k-Tn7 CAST system is described in Strecker et al. Science, 2019 365:48-53, which is incorporated herein by reference.

In one example embodiment, the CAST system is a Mu CAST system, wherein the transposase comprises one or more polypeptides of a Mu transposase. An example Mu CAST system is disclosed in WO/2021/041922 which is incorporated herein by reference.

In one example embodiment, the CAST comprise a catalytically inactive Type II Cas polypeptide (e.g. dCas9) fused to one or more polypeptides of a Tn5 transposase. In another example embodiment, the CAST system comprises a catalytically inactive Type II Cas polypeptide (e.g. dCas9) fused to a piggyback transposase.

### Donor Polynucleotides

The system may further comprise one or more donor polynucleotides (e.g., for insertion into the target polynucleotide). A donor polynucleotide may be an equivalent of a transposable element that can be inserted or integrated to a target site. The donor polynucleotide may be or comprise one or more components of a transposon. A donor polynucleotide may be any type of polynucleotides, including, but not limited to, a gene, a gene fragment, a non-coding polynucleotide, a regulatory polynucleotide, a synthetic polynucleotide, etc. The donor polynucleotide may include a transposon left end (LE) and transposon right end (RE). The LE and RE sequences may be endogenous sequences for the CAST used or may be heterologous sequences recognizable by the CAST used, or the LE or RE may be synthetic sequences that comprise a sequence or structure feature recognized by the CAST and sufficient to allow insertion of the donor polynucleotide into the target polynucleotides. In certain example embodiments, the LE and RE sequences are truncated. In certain example embodiments may be between 100-200 bps, between 100-190 base pairs, 100-180 base pairs, 100-170 base pairs, 100-160 base pairs, 100-150 base pairs, 100-140 base pairs, 100-130 base pairs, 100-120 base pairs, 100-110 base pairs, 20-100 base pairs, 20-90 base pairs, 20-80 base pairs, 20-70 base pairs, 20-60 base pairs, 20-50 base pairs, 20-40 base pairs, 20-30 base pairs, 50 to 100 base pairs, 60-100 base pairs, 70-100 base pairs, 80-100 base pairs, or 90-100 base pairs in length

The donor polynucleotide may be inserted at a position upstream or downstream of a PAM on a target polynucleotide. In some embodiments, a donor polynucleotide comprises a PAM sequence. Examples of PAM sequences include TTTN, ATTN, NGTN, RGTR, VGTD, or VGTR.

The donor polynucleotide may be inserted at a position between 10 bases and 200 bases, e.g., between 20 bases and 150 bases, between 30 bases and 100 bases, between 45 bases and 70 bases, between 45 bases and 60 bases, between 55 bases and 70 bases, between 49 bases and 56 bases or between 60 bases and 66 bases, from a PAM sequence on the target polynucleotide. In some cases, the insertion is at a position upstream of the PAM sequence. In some cases, the insertion is at a position downstream of the PAM sequence. In some cases, the insertion is at a position from 49 to 56 bases or base pairs downstream from a PAM sequence. In some cases, the insertion is at a position from 60 to 66 bases or base pairs downstream from a PAM sequence.

The donor polynucleotide may be used for editing the target polynucleotide. In some cases, the donor polynucleotide comprises one or more mutations to be introduced into the target polynucleotide. Examples of such mutations include substitutions, deletions, insertions, or a combination thereof. The mutations may cause a shift in an open reading frame on the target polynucleotide. In some cases, the donor polynucleotide alters a stop codon in the target polynucleotide. For example, the donor polynucleotide may correct a premature stop codon. The correction may be achieved by deleting the stop codon or introduces one or more mutations to the stop codon. In other example embodiments, the donor polynucleotide addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment refers to less than the entire copy of a gene by providing sufficient nucleotide sequence to restore the functionality of a wild type gene or non-coding regulatory sequence (e.g. sequences encoding long non-coding RNA). In certain example embodiments, the systems disclosed herein may be used to replace a single allele of a defective gene or defective fragment thereof. In another example embodiment, the systems disclosed herein may be used to replace both alleles of a defective gene or defective gene fragment. A “defective gene” or “defective gene fragment” is a gene or portion of a gene that when expressed fails to generate a functioning protein or non-coding RNA with functionality of a corresponding wild-type gene. In certain example embodiments, these defective genes may be associated with one or more disease phenotypes. In certain example embodiments, the defective gene or gene fragment is not replaced but the systems described herein are used to insert donor polynucleotides that encode gene or gene fragments that compensate for or override defective gene expression such that cell phenotypes associated with defective gene expression are eliminated or changed to a different or desired cellular phenotype.

In certain embodiments of the invention, the donor may include, but not be limited to, genes or gene fragments, encoding proteins or RNA transcripts to be expressed, regulatory elements, repair templates, and the like. According to the invention, the donor polynucleotides may comprise left end and right end sequence elements that function with transposition components that mediate insertion.

In certain cases, the donor polynucleotide manipulates a splicing site on the target polynucleotide. In some examples, the donor polynucleotide disrupts a splicing site. The disruption may be achieved by inserting the polynucleotide to a splicing site and/or introducing one or more mutations to the splicing site. In certain examples, the donor polynucleotide may restore a splicing site. For example, the polynucleotide may comprise a splicing site sequence.

The donor polynucleotide to be inserted may have a size from 10 bases to 50 kb in length, e.g., from 50 to 40 kb, from 100 to 30 kb, from 100 bases to 300 bases, from 200 bases to 400 bases, from 300 bases to 500 bases, from 400 bases to 600 bases, from 500 bases to 700 bases, from 600 bases to 800 bases, from 700 bases to 900 bases, from 800 bases to 1000 bases, from 900 bases to from 1100 bases, from 1000 bases to 1200 bases, from 1100 bases to 1300 bases, from 1200 bases to 1400 bases, from 1300 bases to 1500 bases, from 1400 bases to 1600 bases, from 1500 bases to 1700 bases, from 600 bases to 1800 bases, from 1700 bases to 1900 bases, from 1800 bases to 2000 bases, from 1900 bases to 2100 bases, from 2000 bases to 2200 bases, from 2100 bases to 2300 bases, from 2200 bases to 2400 bases, from 2300 bases to 2500 bases, from 2400 bases to 2600 bases, from 2500 bases to 2700 bases, from 2600 bases to 2800 bases, from 2700 bases to 2900 bases, or from 2800 bases to 3000 bases in length.

The components in the systems herein may comprise one or more mutations that alter their (e.g., the transposase(s)) binding affinity to the donor polynucleotide. In some examples, the mutations increase the binding affinity between the transposase(s) and the donor polynucleotide. In certain examples, the mutations decrease the binding affinity between the transposase(s) and the donor polynucleotide. The mutations may alter the activity of the Cas and/or transposase(s).

In certain embodiments, the systems disclosed herein are capable of unidirectional insertion, that is the system inserts the donor polynucleotide in only one orientation.

### B3GNT2 Example Target Modifications

B3GNT2-specific mutations may be introduced using the gene editing systems described above B3GNT2 structure, including human B3GNT2, has been elucidated and active site mutations reducing or eliminating polylactosamine synthesis activity have been performed (Hao, Y et al. J Biol. Chem. (2021), 296 100042; Kadirvelraj, R. et al. (2021), J. Biol. Chem. Research 296 100110). These studies evaluated the importance of specific residues in catalysis and substrate recognition and are expressly incorporated herein by reference. Prior studies by Okada, Y. et al. (2012), Reveille, J (2010) and Tsoi, L. (2012) implicated B3GNT2 as having a crucial role in immune responses involved in polylactosamine synthesis. Those studies supported the connection of B3GNT2 and immune system function in genome-wide association studies that revealed single nucleotide polymorphisms reduced expression of B3GNT2 and were associated with autoimmune diseases including rheumatoid arthritis, ankylosing spondylitis and psoriasis in different populations. Hao et al. reconstituted B3GNT2-deficient Jurkat cells with various point mutations and assessed polylactosamine levels via flow cytometry using the LEA lectin derived from Lycopersicon esculentum. CRISPR-mediated deletion of B3GNT2 in Jurkat cells resulted in a significant decrease in LEA binding relative to wildtype (WT) cells demonstrating B3GNT2 as the major N-acetylglucosaminyltransferase involved in polylactosamine biosynthesis. LEA staining in B3GNT2 KO cells could be recovered by retroviral overexpression if WT B3GNT2 but not empty GFP vector alone. In contrast, reconstitution of KO cells with point mutations within residues required for metal binding (D247A, H376Q, H376L, H376E), substrate binding (K149A, D245A, Y289F, D332A), or within the active site base (D333N) failed to restore cell surface polylactosamine levels, suggesting significantly reduced enzyme activity. Ala279 from the B5-B6 loop is less than 4 Å from the acetyl group of the GlcNAc of the acceptor substrate. A mutation of Ala279 to either Val or Leu may create steric hindrance with the GlcNAc and failed to restore LEA staining when expressed in KO cells. In comparison, expression of a A279G point mutant partially restored polylactosamine levels, although not as robustly as WT B3GNT2. Ala279 is conserved among the seven B3GNTs (B3GNT2-B3GNT8) except for B3GNT3 and B3GNT6 where it is replaced by a Val and a Ser, respectively.

In an embodiment, a programmable nuclease is used to generate an edited B3GNT2 protein that results in a reduction in polylactosamine synthesis (i.e., reduction in B3GNT2 activity). In an embodiment, B3GNT2 is edited to contain point mutations within residues required for metal binding (D247A, H376Q, H376L, H376E), any of which fails to produce polylactosamine or restore cell surface polylactosamine levels in living cells as a consequence of significantly reduced or no B3GNT2 enzyme activity (e.g., base editing). In an embodiment, B3GNT2 is edited to contain point mutations within residues required for substrate binding (K149A, D245A, Y289F, D332A), any of which fails to produce polylactosamine or restore cell surface polylactosamine levels in living cells as a consequence of significantly reduced or no B3GNT2 enzyme activity (e.g., base editing). In an embodiment, B3GNT2 is edited to contain a point mutation within the active site base (e.g., D333N), which fails to produce polylactosamine or restore cell surface polylactosamine levels in living cells as a consequence of significantly reduced or no B3GNT2 enzyme activity (e.g., base editing). In an embodiment, B3GNT2 is edited to contain a mutation of Ala279 from the β5-β6 loop of the B3GNT2 protein, which is less than 4 Å from the acetyl group of the GlcNAc of the acceptor substrate, to either a Val or Leu to create steric hindrance with the GlcNAc, resulting in a B3GNT2 protein which is unable to synthesize polylactosamine as a consequence of significantly reduced or no B3GNT2 enzyme activity.

### MCL1 Example Target Modifications

MCL1 is an anti-apoptotic protein of the BCL-2 family that is essential for the survival of multiple cell lineages and that is highly amplified in human cancer. Under physiological conditions, MCL1 expression is tightly regulated at multiple levels, involving transcriptional, post-transcriptional and post-translational processes. Initial studies of MCL1 identified that its expression was growth-factor dependent in many situations, and that it was capable of protecting cells from growth factor withdrawal-induced apoptosis (Bodrug, S. E. et al. Cell Death Diff. (1995), 2(3), 173-182). Ubiquitination of MCL1, that targets it for proteasomal degradation, allows for rapid elimination of the protein and triggering of cell death, in response to various cellular events. MCL1-specific mutations may be introduced using the gene editing systems described above. BCL-2-related proteins all contain at least one of the four conserved BCL-2 homology domains (BH1-BH4), which enable protein-protein interactions between the different members of the family. Proteins of the BCL-2 family display either anti-apoptotic or pro-apoptotic functions. The members that inhibit apoptosis include Bcl-2, Bcl-XL, MCL1, (BCL2A1; Bfl-1/A1), BCL-B and BCL-W. These anti-apoptotic proteins and the pro-apoptotic effector members, such as Bax and Bak, share at least three BH domains and a similar globular structure. These two groups are thus named multi-domain proteins and they mainly reside at the mitochondria. (Moldoveanu, T. et al. Trends Biochem. Sci. (2014), 39, 101-111).

A growing list of trophic factors has been shown to induce transcriptional upregulation of MCL1, including cytokines such as the interleukins IL-3, IL-5, IL-6, and granulocyte-macrophage colony-stimulating factor (GM-CSF), as well as growth factors such as epidermal growth factor (EGF) and vascular endothelial growth factor (VEGF). Intracellular regulation of MCL1 transcription is mediated by a number of externally activated and constitutively activated transcription factors, notably the signal transducers and activators of transcription (STAT) family. The promoter region of MCL1, characterized by Akgul et al. (Cell Mol. Life Sci. 57(4), 684-691), contains an array of putative and confirmed transcription factor binding sites, including consensus STAT response elements, cAMP response elements (CRE), and nuclear factor kappaB binding sites. Both STAT3, in response to IL-6, VEGF and IL-3, and STAT5, in response to Bcr-Abl signaling in chronic myeloid leukemia, have been shown to upregulate MCL1 transcription. It has been suggested that STAT3 activation by phosphorylation is absolutely required for MCL1-mediated macrophage survival (Liu, H. et al. Blood (2003), 102(1), 344-352). Other confirmed transcription factors known to upregulate MCL1 expression include cAMP response element binding protein, PU.1, SP1, and under hypoxic conditions, hypoxia-inducible factor 1a (Liu X. H., et al. J. Cell Biochem. (2006), 97(4), 755-765). Conversely, MCL1 may be down-regulated transcriptionally under a number of conditions, particularly under growth factor withdrawal, and on the induction of apoptosis induced by a variety of treatments, including staurosporine and UV exposure (Nijhawan, D. et al. Genes Dev. (2003), 7(12), 1475-1486). In most cases, down-regulation is mediated by inactivation of the transcription factors stimulating MCL1 transcription, but the MCL1 promoter is directly repressed by the binding of the E2F-1 transcription factor. Unlike other BCL-2-related survival proteins, MCL1 harbors a long unstructured N-terminus that appears to be involved in different post-translational modifications of MCL1 (Le Gouill, S., et al. Cell Cycle (2004), 3, 1259-1262). For example, it has been shown that, during apoptosis, MCL1 protein can be cleaved by caspases and granzyme B, at two distinct sites (Asp127 and Asp157) within the N-terminus (Han, J. et al. J. Biol. Chem. (2005), 280, 16383-16392; Herrant, M., et al. Oncogene (2004), 23, 7863-7873). Some studies reported that cleavage at these sites impairs the anti-apoptotic properties of MCL1, or even converts it into a pro-apoptotic protein (Han, J. et al. J. Biol. Chem. (2005), 280, 16383-16392; Micheals, J. et al. Oncogene (2004), 23, 4818-4827; Herrant, M., et al. Oncogene (2004), 23, 7863-7873). Cleavage of MCL1 thus appears to be a process through which apoptotic cells can inactivate residual MCL1 that could act as a brake to the achievement of cell death. The N-terminus of MCL1 contains PEST regions (Kozopas, K. M. et al., Proc. Natl. Acad. Sci. (1993), 90, 3516-3520), enriched in proline (P), glutamate (E), serine (S) and threonine (T) residues, which are common features of rapidly degraded proteins (Rechsteiner, M. et al. Trends Biochem. Sci. (1996), 21, 267-271). These regions contain the two caspase cleavage sites of MCL1 and many phosphorylation sites. Differential phosphorylation of MCL1 at specific sites has been reported to result in different outcomes. For example, the cell cycle-dependent phosphorylation of Ser64 by CDK1, CDK2 and JNK1 enhances the anti-apoptotic function of MCL1 by increasing its interaction with pro-apoptotic members of the BCL-2 family, without modifying its half-life (Kobayashi, S. et al. J. Biol. Chem. (2007), 282, 18407-18417). Two independent groups have also shown that MCL1 phosphorylation at Thr92 and Thr163, stimulated by TPA-induced ERK activation, stabilizes MCL1 in some cancer cell lines (Domina, A. M. et al. Oncogene (2004), 23, 5301-5315; Ding, Q. Cancer Res. (2008), 68, 6109-6117; Nifoussi, S. K. PloS One (2012), 7, e47060). Likewise, Ser121 and Thr163 have been found to be phosphorylated by JNK (Inoshita, S., J. Biol. Chem. (2002), 277, 43730-43734; Kodama, Y. et al., Gastroenterology 136, 1423-1434). In hepatocytes, this JNK-mediated phosphorylation stabilizes MCL1 and affords protection against TNF-induced apoptosis, whereas in endothelial cells submitted to oxidative stress, this phosphorylation reduces the anti-apoptotic effect of MCL1 (Inoshita, S., J. Biol. Chem. (2002), 277, 43730-43734; Kodama, Y. et al., Gastroenterology 136, 1423-1434). Moreover, phosphorylation of MCL1 at Ser155, Ser159 and Thr163, in a different cellular context, has been shown to favor MCL1 degradation by the ubiquitin-proteosome system (UPS).

In an embodiment, a gene editing system as described above is used to generate an edited MCL1 gene that results in a reduction or inhibition in MCL1 expression or activity. In an embodiment, MCL1 is edited to contain mutations including point mutations, deletions and insertions within the MCL1 promoter region required for upregulation of expression by an array of putative and confirmed transcription factors, including consensus STAT response elements, cAMP response elements (CRE), and nuclear factor kappaB binding sites, which lead to a reduction or inhibition in MCL1 expression because the transcription factor(s) can no longer bind efficiently to the MCL1 promoter region. Both STAT3, in response to IL-6, VEGF and IL-3, and STAT5, in response to Ber-Abl signaling in chronic cancer (e.g. myeloid leukemia), have been shown to upregulate MCL1 transcription. It has been suggested that STAT3 activation by phosphorylation is absolutely required for MCL1-mediated macrophage survival. In an embodiment, MCL1 is edited to contain mutations including point mutations, deletions and insertions within the MCL1 promoter region that reduces or inhibits STAT3 binding which leads to a reduction or inhibition in MCL1 expression or activity. In an embodiment, editing is used to mutate one or more transcription factor genes that stimulate MCL1 transcription. In an embodiment, the MCL1 transcription factors STAT3, cAMP response element binding protein, PU.1, SP1 and hypoxia-inducible factor 1a are edited and mutated, which leads to a reduction or inhibition of MCL1 expression or activity because the above transcription factors, either alone and in any combination thereof, are inactivated.

The MCL1 promoter is directly repressed by the binding of the E2F-1 transcription factor. In an embodiment, MCL1 expression and activity is decreased by editing and imparting mutations in the E2F-1 transcription factor whereby E2F-1 binding is increased by stabilizing the binding interaction between E2F-1 to the MCL1 promoter region, which leads to a reduction or inhibition in MCL1 expression and activity.

As disclosed herein, MCL1 protein can be cleaved by caspases and granzyme B at two distinct sites (Asp127 and Asp157) within the N-terminus and it appears that cleavage at these sites impairs the anti-apoptotic properties of MCL1, or even converts it into a pro-apoptotic protein. Cleavage of MCL1 thus appears to be a process through which apoptotic cells can inactivate residual MCL1, which in turn could act as a brake on apoptosis. In an embodiment, MCL1 is edited to contain one or more mutations to stabilize the Asp127 and Asp157 cleavage sites such that caspases and granzyme cleave the MCL1 Asp127 and Asp157 sites more efficiently during apoptosis leading to more cancer cell death.

As disclosed herein, the N-terminus of MCL1 contains PEST regions, which as described above are enriched in proline (P), glutamate (E), serine (S) and threonine (T) residues and are common features of rapidly degraded proteins. These regions contain the two caspase cleavage sites of MCL1 (Asp127 and Asp 157 discussed above) and many phosphorylation sites. Differential phosphorylation of MCL1 at specific sites has been reported to result in different outcomes. For example, the cell cycle-dependent phosphorylation of Ser64 by CDK1, CDK2 and JNK1 enhances the anti-apoptotic function of MCL1 by increasing its interaction with pro-apoptotic members of the BCL-2 family, without modifying its half-life. In an embodiment, MCL1 is edited at Ser64 which decreases or eliminates phosphorylation of the Ser64 residue which in turn reduces the anti-apoptotic function of MCL1 by reducing its interaction with pro-apoptotic members of the BCL-2 family. As discussed above, it has been shown that MCL1 phosphorylation at Thr92 and Thr163, stimulated by TPA-induced ERK activation, stabilizes MCL1 in some cancer cell lines. In an embodiment, MCL1 is edited at Thr92 and Thr163, which reduces phosphorylation at these two residues and leads to a reduction in MCL1 activity because the MCL1 protein is destabilized. Likewise, Ser121 and Thr163 have been found to be phosphorylated by JNK. In hepatocytes, JNK-mediated phosphorylation stabilizes MCL1 and affords protection against TNF-induced apoptosis, whereas in endothelial cells submitted to oxidative stress, this phosphorylation reduces the anti-apoptotic effect of MCL1. In an embodiment, MCL1 is edited and mutated at Ser121 and Thr163, which reduces phosphorylation at these two residues and leads to a reduction in MCL1 stabilization and affords a reduction in protection against TNF-induced apoptosis. Moreover, phosphorylation of MCL1 at Ser155, Ser159 and Thr163, in a different cellular context, has been shown to favor MCL1 degradation by the ubiquitin-proteosome system (UPS). In an embodiment, MCL1 is edited at one or more of Ser155, Ser 159 and Thr163, which increases phosphorylation at one or more of these residues and increases MCL1 degradation by the ubiquitin-proteosome system (UPS).

### BCL2A1 Example Target Modifications

The transcription of BCL2A1 is highly regulated. It was originally identified by as a gene induced by GM-CSF and LPS, suggesting that it may be an early-response gene. Later on it was found to be inducible by tumor necrosis factor A and identified as an NF-κB target gene (Zong, W. et al. Genes Dev (1999), 13, 382-387). Simultaneously, BCL2A1 transcription was reported to be induced in response to antigen receptor stimulation. Subsequently, several reports have demonstrated the importance of BCL2A1 upregulation for B-lymphocyte survival upon CD40 signaling (Lee, H. et al. Proc. Natl. Acad. Sci. (1999), 96, 9136-9141). In addition to CD40 signaling, PI3K and ERK signaling initiated by ICAM-1 binding have been found to induce NF-κB and subsequently BCL2A1 expression. Interestingly, both hyperoxia and low levels of reactive oxygen species were described to increase BCL2A1 transcription, possibly in an NF-κB-dependent manner (Kim, H. et al. Oncogene (2005), 24, 1252-1261). In both situations, BCL2A1 exerted a pro-survival function to prevent cell death. Besides NF-κB, several other transcription factors have been implicated in BCL2A1 transcriptional regulation, including all-trans retinoic acids or retinoic X receptor agonists, the (_EX5/_KTS) isoform of WT-142 and the transcriptional enhancer Spi-1/PU.1. On the other hand, BCL2A1 transcription is repressed by the plasma cell transcription factor PRDI-BF1/Blimp-1 (Shaffer, A. et al. Immunity (2002), 17, 51-62). In addition to the transcriptional regulation, BCL2A1 is also controlled at the post-translational level. In this regard, BCL2A1 is regulated by the ubiquitin/proteasome pathway and undergoes constitutive proteasome-mediated turnover, resulting in a short half-life of the protein. However, thus far no E3-ligase for BCL2A1 has been identified. Whether the proteasomal degradation of BCL2A1 can also be controlled by certain pro- or anti-apoptotic stimuli, for example, via phosphorylation events, has not been investigated yet but may provide an extra layer of regulation. In addition to the proteasomal turnover, cleavage by m-calpain can convert BCL2A1 from an anti-into a pro-apoptotic protein (Kucharczak, J. et al. Cell Death Diff. (2005), 12, 1225-1239).

BCL2A1 shares all four BH-domains with BCL-2. Of these, the BH1 and BH2 domains are highly conserved while the BH3 and in particular the BH4 domains have little homology with those of the cognate anti-apoptotic proteins. Similar to the other anti-apoptotic BCL-2 proteins, BCL2A1 consists of 8 a-helixes. In particular, the helixes α4, α5 and α6, corresponding to the BH3, BH1 and BH2 domains, respectively, form a groove on the protein surface that is able to interact with the BH3 domain of the pro-apoptotic BCL-2 family proteins (Smits, C. et al. Structure (2008), 16, 818-829). Despite the overall structural homology, BCL2A1 differs from all the other anti-apoptotic BCL-2 family members in its C-terminal end (Herman, M. et al. FEBS Letters (2008), 582, 3590-3594). While BCL-2, BCL-XL, BCL-W and MCL1 localize predominantly to inner cellular membranes via their hydrophobic region at the C-terminal end, the C-terminal portion of BCL2A1 contains a hydrophilic stretch responsible for ubiquitination and degradation via the proteosome pathway. Interestingly among all the pro-survival family members only BCL2A1 and MCL1 show such a rapid turnover rate. The half-life of BCL2A1 and MCL1 is estimated to be as short as 30 min while the half-life of BCL-2 is estimated to be around 24 h (Herold, J. et al. J. Biol. Chem., (2006), 281, 13663-13671). Whether BCL2A1 membrane association can be equated with insertion, as reported for BCL-W, remains to be investigated. Of note, BCL2A1 may interact with all BH-123 proteins, including Bok, as well as several BH3-only proteins. In a second study using in vitro translated protein, human A1/Bfl-1 was found to co-immunoprecipitate only with Bax when its transmembrane domain was deleted (Zhang, H. et al. J. Biol. Chem. (2000), 275, 11092-11099). Other studies performed in a more physiological context, reported a strong association between both human and murine BCL2A1 and endogenous Bak but not with Bax (Simmons, M. Oncogene (2008), 27, 1421-1428). BCL2A1 with Bax or Bak reflects a preferential binding affinity that can be found also in the other pro-survival members of the family. In general, the anti-apoptotic proteins can be subdivided depending on their ability to interact strongly with either Bax or Bak. The BH3-only peptide of Bax is able to interact with high affinity with BCL-2 and BCL-W, while Bak peptide binds potently Bcl-xL, MCL1 and BCL2A1 (Willis, S. et al. Genes Dev. (2005), 19, 1294-1305; Ku, B. et al. Cell Res. (2010), 21, 627-641). BCL2A1 interacts with different affinity also to the BH3-only proteins. Competitive binding assays using BH3-peptides show that BCL2A1, similarly to MCL1, has no affinity for Bad whereas it binds very tightly to Bim, Bid and BBC3 (PUMA); while BCL-2, Bcl-xL and Bcl-w interact potently with Bad, Bmf, Bim and BBC3 (PUMA) (Chen, L. et al. Mol. Cell (2005), 17, 393-403). The specific interaction pattern of BCL2A1 is probably due to the presence of an acidic residue, glutamate 78, within the binding groove that serves as an interaction surface for the BH3-domains of the pro-apoptotic BCL-2 family members (Ottina, E. et al. Exp. Cell Res. (2012), 318, 1291-1313). In contrast, all other anti-apoptotic proteins have a hydrophobic or slightly positively charged binding groove. Moreover, BCL2A1 can be stabilized by the interaction with Bim (Herold M. et al. J. Biol. Chem. (2006), 281, 13663-13671). The ability to be stabilized by BH3-only proteins and its cytosolic localization suggest that mouse BCL2A1 might act as a first barrier against premature or faulty activation of the apoptotic machinery. However, this feature does not seem to be conserved, as human BCL2A1 can target mitochondria, although it is unclear if it could also do so in the absence of pro-apoptotic BCL-2 family proteins.

The Bfl-1-Bim-BH3 peptide-binding pocket has similar overall properties as the corresponding groove in other BCL-2 proteins. Several hydrophobic patches line along the pocket at positions conserved in the BCL-2 proteins These hydrophobic patches interact with highly conserved residues on the amphipathic helix of BH3-only proteins. In the Bim peptide these residues are represented by Ile148, Leu152, Ile155 and Phe159. The Bim peptide makes two additional hydrophobic interactions; Trp147 of Bim stacks onto a surface patch formed by Leu52 and Cys55 of Bfl-1, and Tyr163 of Bim makes hydrophobic interactions with Phe148 and Val40 of BCL2A1(Bfl-1) (Herman, M. et al. FEBS Letters (2008), 582, 3590-3594). Since these latter residues are highly conserved in other anti-apoptotic BCL-2 proteins, this region is labeled the h5 patch. The part of the pocket lined by helix a4 and a5 has several polar interactions similar to those in other BCL-2 proteins. The most notable is the conserved Arg88 of the WGR motif in BH1, which interacts with the strictly conserved Asp157 of Bim. A characteristic feature of the Bfl-1 BH3-binding groove is the higher negative charge in its central region, contributed by Glu78 and Glu80 at the end of helix a4 (Herman, M. et al. FEBS Letters (2008), 582, 3590-3594).

In an embodiment, a gene editing system is used to generate an edited BCL2A1 gene that results in a reduction or inhibition in BCL2A1 expression or activity. In an embodiment, BCL2A1 is edited to contain mutations including point mutations, deletions and insertions within the BCL2A1 promoter region required for upregulation of expression by an array of putative and confirmed transcription factors, in which CD40 signaling, PI3K and ERK signaling initiated by ICAM-1 binding have been found to induce NF-κB and subsequently BCL2A1 expression. Further, both hyperoxia and low levels of reactive oxygen species were described to increase BCL2A1 transcription, possibly in an NF-κB-dependent manner. In an embodiment, the promoter region involved in binding the transcription activating factor NF-kB is mutated, which leads to a reduction or inhibition in BCL2A1 expression and activity because the NF-κB transcription factor can no longer bind efficiently to the BCL2A1 promoter region. Besides NF-κB, several other transcription factors have been implicated in BCL2A1 transcriptional regulation, including all-trans retinoic acids or retinoic X receptor agonists, the (_EX5/_KTS) isoform of WT-142 and the transcriptional enhancer Spi-1/PU.1. In an embodiment, the BCL2A1 promoter region involved in binding the transcription factors trans retinoic acids or retinoic X receptor agonists, the (_EX5/_KTS) isoform of WT-142 and the transcriptional enhancer Spi-1/PU.1 is mutated which leads to a reduction or inhibition in BCL2A1 expression and activity because the transcription factors can no longer bind efficiently to the BCL2A1 promoter region. Alternatively, BCL2A1 transcription is repressed by the plasma cell transcription factor PRDI-BF1/Blimp-1. In an embodiment, the BCL2A1 promoter region involved in binding transcription factor PRDI-BF1/Blimp-1 is mutated, which leads to an increase or improvement in BLIMP-1 binding and consequently increased repression of BCL2A1 expression and activity because the transcription factor binds more efficiently to the BCL2A1 promoter region.

As described herein, competitive binding assays using BH3-peptides show that BCL2A1, similarly to MCL1, has no affinity for Bad whereas it binds very tightly to Bim, Bid andBBC3 (PUMA); while BCL-2, Bcl-xL and Bcl-w interact potently with Bad, Bmf, Bim andBBC3 (PUMA). The specific interaction pattern of BCL2A1 is probably due to the presence of an acidic residue, glutamate 78, within the binding groove that serves as an interaction surface for the BH3-domains of the pro-apoptotic BCL-2 family members. In an embodiment, BCL2A1 protein activity is decreased by editing and imparting mutations corresponding to the acidic residue located at glutamate 78 (Glu78) in the native protein within the binding groove, which leads to a decrease in BCL2A1 activity due to a reduced binding interaction with BH3 peptide domains.

As described herein, the Bfl-1-Bim-BH3 peptide-binding pocket has similar overall properties as the corresponding groove in other BCL-2 proteins. Several hydrophobic patches line along the pocket at positions conserved in the BCL-2 proteins. These hydrophobic patches interact with highly conserved residues on the amphipathic helix of BH3-only proteins. In the Bim peptide these residues are represented by Ile148, Leu152, Ile155 and Phe159. In an embodiment, BCL2A1 protein activity is decreased by editing and imparting substitution mutations corresponding to the hydrophobic patches that interact with one or more highly conserved residues on the amphipathic helix of BH3-only proteins for example, the Bim peptide where these residues are represented by Ile148, Leu152, Ile155 and Phe159, which leads to a decrease in BCL2A1 activity due to a reduced binding interaction with BH3 (e.g., Bim) peptide domains. The Bim peptide makes two additional hydrophobic interactions with BCL2A1. Trp147 of Bim stacks onto a surface patch formed by Leu52 and Cys55 of BCL2A1 and Tyr163 of Bim makes hydrophobic interactions with Phe148 and Val40 of BCL2A1. In an embodiment, BCL2A1 protein activity is decreased by editing and imparting mutations corresponding to one or more of the additional hydrophobic regions that interact with highly conserved residues Trp147 of Bim, Leu52 and Cys55 of BCL2A1 and Tyr163 of Bim and makes hydrophobic interactions with Phe148 and Val40, of BCL2A1 which leads to a decrease in BCL2A1 activity due to a reduced binding interaction with Bim peptide domains.

These Phe148 and Val40 residues are highly conserved in other anti-apoptotic BCL-2 proteins. The part of the pocket lined by helix a4 and a5 has several polar interactions similar to those in other BCL-2 proteins. The most notable is the conserved Arg88 of the WGR motif in BH1, which interacts with the strictly conserved Asp157 of Bim. In an embodiment, BCL2A1 protein activity is decreased by editing and imparting substitution mutations corresponding to Arg88 of the WGR motif in BH1, which leads to a reduction in BCL2A1 activity because the polar interactions between Arg88 and Asp157 are reduced or eliminated.

### JUNB Example Target Modifications

The product of the junB gene is a member of the AP-1 family of transcription factors that activates transcription by binding to TPA-responsive elements (TREs) within the promoters of target genes. Components of AP-1 are immediate-early genes whose expression is upregulated by a plethora of extracellular stimuli and are important in mediating cellular proliferation and differentiation. Such stimuli include the pleiotropic cytokine interleukin-6 (IL-6) which plays a role in immune and inflammatory responses and ciliary neurotrophic factor (CNTF) which enhances survival and differentiation of neurons and glia. Expression from junB promoter-CAT reporter constructs in HepG2 cells identified a region between −196 and −91 that can mediate response to IL-6 and CNTF and was able to confer responsiveness to a heterologous promoter. It was shown by gel retardation analysis that distinct nuclear factors induced by IL-6 specifically bind to this interleukin-6 response element (IRE) (Lutticken, C. et al. Oncogene (1995), 10(5), 985-994). This region contains both a putative ETS- and a STAT-transcription factor binding site. Further, it was shown by mutational analysis and supershift data that the IL-6 induced complex contains the transcription factor APRF/Stat3 that is both necessary and sufficient for activation. This site does not appear to bind STAT1 itself, as was shown by supershift analysis and a lack of response to IFN-gamma both at the DNA-binding and transcriptional level. Furthermore, it was demonstrated that the junB-IRE-binding activity induced by IL-6 requires tyrosine kinase activity, whereas induced transactivation of IRE-constructs additionally occurs through an H7-sensitive pathway that is p21ras-independent, implicating serine/threonine kinases in the transactivation of IRE-binding factors (Lutticken, C. et al. Oncogene (1995), 10(5), 985-994).

JunB differs considerably from c-Jun in its ability to activate AP-1-responsive genes and induce oncogenic transformation. It has been demonstrated that the decreased ability of JunB to activate gene expression is the result of a small number of amino acid changes between its DNA-binding and dimerization motifs and the corresponding regions of c-Jun (Deng, T. et al. Genes and Dev. (1993), 7:479-490). Changes in its DNA-binding and dimerization motifs led to a 10-fold decrease in the DNA-binding activity of JunB. JunB can be converted into a c-Jun-like activator by substituting four amino acids in its DNA-binding and dimerization motifs with the corresponding c-Jun sequences. JunB can also attenuate trans-activation by c-Jun, an activity mediated by its leucine zipper. This ability depends on two glycine residues that decrease the stability of the JunB leucine zipper, resulting in decreased homodimerization and increased heterodimerization. It has been shown that small changes in primary structure, including chemically conservative changes, can result in functional divergence of two highly related transcriptional regulators.

The role of the DNA-binding domain in JUN proteins, was investigated by generating a restriction site at the sequence surrounding codons 260 of c-Jun and 273 of JunB, which correspond to the exact amino termini of their basic regions (Vogt. P. et al. Adv. Cancer Res. (1990), 55, 1-35). The results indicated that chimeras containing amino acids 1-260 of c-Jun and the JunB DNA-binding domain, CB5, was an inefficient activator of reporter gene expression as with JunB, whereas the reverse chimera BC5, was a potent activator similar to c-Jun. A chimera containing the first 278 amino acids of c-Jun, CB6, whose basic region is derived from c-Jun, but whose leucine zipper is from JunB was two-fold less efficient than c-Jun. Hence, the JunB leucine zipper may not be as effective as the corresponding region of c-Jun. This finding was further supported by the significant increase in activity of the reverse chimera, BC6, which contains mostly JunB sequences with the exception of the leucine zipper derived from c-Jun. In comparison with wild-type JunB, BC6 was 3.5-fold more active. These results indicated that the major determinants of the differential activity of c-Jun and JunB are located in the basic region, which directly contacts DNA, and the leucine zipper, which mediates dimerization (Vinson, C et al., Science et al. (1989), 246: 911-916).

Among the 11-amino acid differences between the two DNA-binding domains, all except two, E293G and N299G, are conservative changes. Importantly, these sequence substitutions result in the presence of two glycines within the leucine zipper of JunB. Glycine and proline residues are helix destabilizers and are discriminated against within leucine zippers (Landschulz, W. et al. Science (1988a), 240: 1759-1764). Therefore, these glycines are likely to decrease the stability of the α-helix formed by the JunB leucine zipper. Replacement of the two glycines of JunB with the corresponding c-Jun sequences, resulted in a four-to-five-fold increase in activity. Replacement of either glycine alone resulted in a smaller increase in JunB activity. Two of the other sequence differences, I264L and S267T, between the DNA-binding domains of c-Jun and JunB, reside in the basic region. Despite the conserved nature of these changes, replacement of the JunB sequences with the corresponding c-Jun sequences (L264I/T267S), resulted in a six-to-seven-fold increase in activity. The effect of the single substitutions was smaller, with T267S being more effective than L264I. Combination of the two basic region substitutions with the two leucine zipper substitutions resulted in a 12-fold increase in JunB activity, reaching almost the same level of activity as c-Jun. To confirm the importance of the two glycine substitutions within the leucine zipper, three other positions within the JunB leucine zipper were converted to the corresponding c-Jun sequences (A292S, S295A, and A297T). Individually, none of these substitutions had any effect on JunB activity (Deng, T, et al., Genes and Dev. (1993), 7, 479-490).

In an embodiment, a gene editing system as described above is used to generate an edited JUNB gene that results in a reduction or inhibition in JUNB expression or activity. In an embodiment, JUNB is edited to contain mutations including point mutations, deletions and insertions within the JUNB promoter region required for upregulation of expression by an array of putative and confirmed AP-1 family of transcription factors that activate transcription by binding to TPA-responsive elements (TREs) within the promoter region of JUNB. In an embodiment, the JUNB promoter region involved in binding the AP-1 family of transcription factors that activates transcription by binding to TPA-responsive elements (TREs) is mutated which leads to a reduction or inhibition in JUNB expression and activity because the binding of the transcription factors is decreased or inhibited at the JUNB promoter region.

In an embodiment, a programmable nuclease is used to generate an edited JUNB gene in the leucine zipper basic region which leads to a reduction in JUNB activity. In an embodiment, the gene edited mutation leads to an altered protein sequence with substitutions occurring from E293G and N299G to E293P and N299G, or E293G and N299P, or E293P and N299P. Importantly, any of these sequence substitutions result in the presence of one glycine and one proline or two prolines within the leucine zipper of JunB. Glycine and proline residues are known helix destabilizers and are discriminated against within leucine zippers. In an embodiment, the edited JUNB protein containing substitutions from residues E293G and N299G to E293P and N299G lead to a reduction in JUNB activity because the mutations decrease dimerization of the JUNB proteins. In an embodiment, the edited JUNB protein containing substitutions from residues E293G and N299G to E293G and N299P lead to a reduction in JUNB activity because the mutations decrease dimerization of the JUNB proteins. In an embodiment, the edited JUNB protein containing substitutions from residues E293G and N299G to E293P and N299P lead to a reduction in JUNB activity because the mutations decrease dimerization of the JUNB protein. Replacement of the two glycines of JunB with the corresponding c-Jun sequences, resulted in a four-to-five-fold increase in activity, indicating the importance of these residues on functional activity. Two of the other sequence differences, I264L and S267T, between the DNA-binding domains of c-Jun and JunB, reside in the basic region. Despite the conserved nature of these changes, replacement of the JunB sequences with the corresponding c-Jun sequences (L264I/T267S), resulted in a six-to-seven-fold increase in activity. In an embodiment, the edited JUNB protein containing substitutions in the basic region at positions 1264 and S267 lead to a reduction in JUNB activity because the basic region amino acids 1264 and S267 have been mutated. In an embodiment, the edited JUNB protein containing substitutions in the basic region at positions 1264 and S267 lead to a reduction in JUNB activity because the basic region amino acids 1264 and S267 have been mutated by substituting to non-conserved amino acids. The effect of the single substitutions was smaller, with T267S being more effective than L264I. In an embodiment, the edited JUNB protein containing a substitution in the basic region containing a single substitution at position I264 leads to a reduction in JUNB activity because the basic region amino acid 1264 has been mutated by substituting to a non-conserved amino acid. In an embodiment, the edited JUNB protein containing a substitution in the basic region containing a single mutation at position T267 leads to a reduction in JUNB activity because the basic region amino acid T267 has been mutated by substituting to a non-conserved amino acid. Combination of the two basic region substitutions with the two leucine zipper substitutions resulted in a 12-fold increase in JunB activity, reaching almost the same level of activity as c-Jun. In an embodiment, the edited JUNB protein containing substitutions in the two basic regions lead to a significant reduction in JUNB activity because the two basic regions amino acids 1264 and T267 and E293 and N299 have been mutated by substituting to non-conserved amino acids at one or more or all of these positions. The importance of the two glycine substitutions within the leucine zipper, were determined at three other positions within the JunB leucine zipper by converting to the corresponding c-Jun sequences at positions A292S, S295A, and A297T. Individually, none of these substitutions had any effect on JunB activity, thus demonstrating the importance of the above described amino acids. (Deng, T, et al., Genes and Dev. (1993), 7, 479-490).

### Method of Enhancing Anti-Tumor Immunity by Inhibiting Poly-LacNac Synthesis

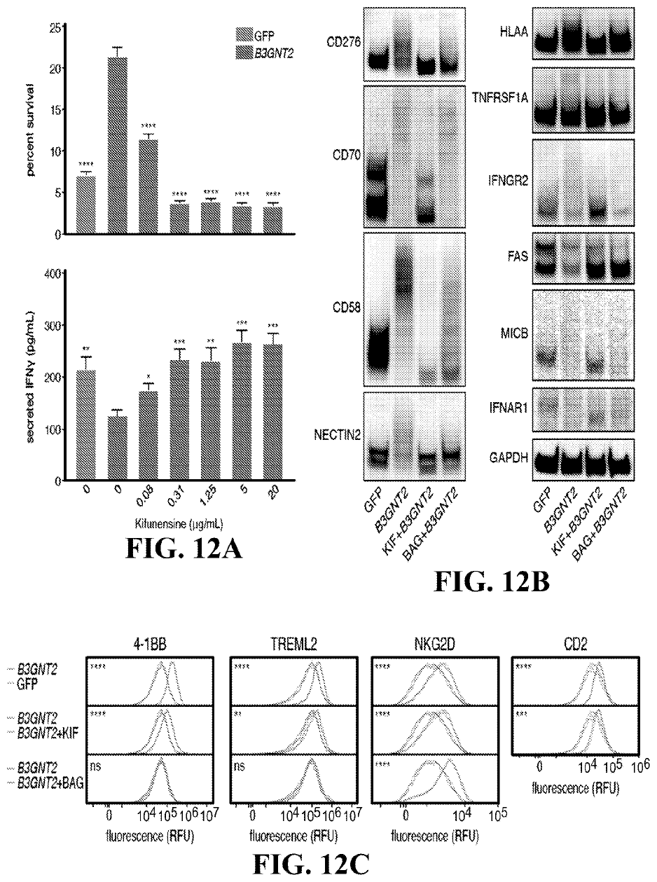

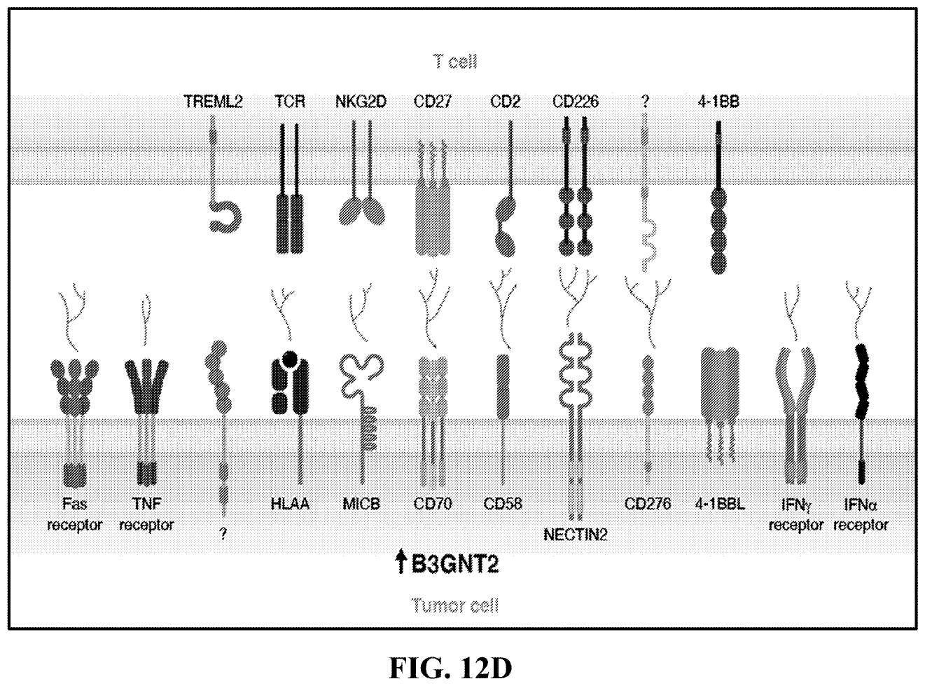

Poly-LacNac is increased on glycoproteins on the surface of tumors to inhibit binding of certain T cell activating surface proteins, thus allowing tumors to evade an anti-tumor immune response (e.g., by overexpressing B3GNT2). Poly-LacNac consists of repeated N-acetyl-lactosamine (Galβ1-4GlcNAc)ₙ residues formed as GlcNAc residues and are attached to galactosyl termini via the enzymatic activity of β-1,3 N-acetylglucosaminyltransferase (B3GNT family, including B3GNT2). B3GNT2 is a beta-1,3-N-acetylglucosaminyltransferase involved in poly-LacNac synthesis that has been suggested to glycosylate PD-1 in T cells and to affect T cell activation (Sun et al. 2020). In the present disclosure, B3GNT2 was shown to promote resistance through an orthogonal pathway by increasing poly-LacNac on at least 10 tumor ligands and receptors (CD276, CD70, CD58, NECTIN2, HLA-A, TNFRSF1A, IFNGR2, FAS, IFNARI, MICB). Increased poly-LacNac was confirmed using the potent inhibitors kifunensine or benzyl-O—N-acetylgalactosamide (BAG). All of these ligands and receptors are N-glycosylated, whereas a subset (CD276, CD58, NECTIN2, IFNGR2, FAS, and IFNAR1) are O-glycosylated (Example 1, FIG. 12). In a prior study investigating the function of B3GNT2, it was shown that B3GNT2 knockout mice have lower polyLacNac on B and T cells, resulting in hyperactivity (Togayachi et al. 2010).

In one example embodiments, one or more agents capable of decreasing Poly-LacNac is administered to a subject to enhance an anti-tumor immune response or an immunotherapy. In certain embodiments, the one or more therapeutics described herein are administered to a subject that has a tumor overexpressing B3GNT2 or has increased poly-LacNac on surface proteins. The present invention also provides for determining subjects that may respond to inhibition of poly-LacNac. For example, the tumor overexpresses B3GNT2 and does not overexpress another protein that allows evasion of an immune response.

### Small Molecule Inhibitors of Poly-LacNac Synthesis

In embodiments, the present disclosure provides methods of enhancing anti-tumor immunity by inhibiting poly-N-acetyl-lactosamine (poly-LacNac) synthesis. Poly-LacNac synthesis can be inhibited by agents that block synthesis of N- or O-linked glycan extension or inhibit a-mannosidase activity. For example, tunicamycin, is a potent inhibitor of N-glycan synthesis and kefunensine inhibits human endoplasmic reticulum a-1,2-mannosidase I and Golgi Class I mannosidases IA, IB and IC with Ki values of 130 and 23 nM, respectively.

In embodiments, poly-LacNac synthesis is inhibited by administering one or more small molecule agents selected from the group consisting of benzyl-O—N-acetylgalactosamide (BAG), kifunensine (KIF), tunicamycin, 3′-Azidothymidine (AZT), 2-acetamido-1,3,6-tri-O-acetyl-4-deoxy-4-fluoro-D-glucopyranose [4-F-GlcNAc], and deoxymannojirimycin (DMN).

### Conjugated Antibodies for Reduction of Poly-LacNAc on Tumor Surface

In another embodiment, the one or more agents comprise an antibody that binds to a tumor-specific marker and is linked to an enzyme capable of cleaving poly-LacNac. The marker can be any tumor specific surface marker or a tumor antigen presented on the surface by class I HLA molecules. The conjugated antibody may be an antibody or antigen binding fragment thereof, chemically linked to one or more enzymes. In a preferred embodiment, an antibody includes a linker that enables attachment or conjugation of the enzyme to the antibody. The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)2, Fabc, Fd, dAb, V_{HH} and scFv and/or Fv fragments.

Non-limiting tumor markers include the following: MR1 (see, e.g., Crowther, et al., 2020, Genome-wide CRISPR-Cas9 screening reveals ubiquitous T cell cancer targeting via the monomorphic MHC class I-related protein MR1, Nature Immunology volume 21, pages 178-185), B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); K-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (ICE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDGlcp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDGlcp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEMI/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukaemia/retinoic acid receptor a); PRAME (preferentially expressed antigen of melanoma); SAGE (sarcoma antigen); TEL/AMLI (translocation Ets-family leukemia/acute myeloid leukemia 1); TPI/m (triosephosphate isomerase mutated); CD70; and any combination thereof.

In certain example embodiments, the enzyme is selected from the group consisting of endo H, endo F₂, endo F₃, peptide:N-glycosidase F (PNGase F), endo D, O-glycosidase, endo-β-galactosidase, sialidase and O-sialoglycoprotease.

### Combination Therapy

The present invention also provides methods comprising combination therapy. As used herein, “combination therapy” includes administration of a therapeutically effective amount of the one or more agents described herein in combination with administering an immunotherapy. In an embodiment, the method further comprises administering an immunotherapy in combination with the one or more agents described herein. In embodiments, the immunotherapy is adoptive cell therapy. In another embodiment, the immunotherapy is checkpoint blockade (CPB) therapy, also referred to as immune checkpoint inhibition (ICI). The therapeutic agents described herein may shift a subject from an immunotherapy nonresponder to a responder. In certain embodiments, a tumor may overexpress any of the targets after administered an immunotherapy. Thus, overexpression of a target may be determined during immunotherapy treatment and thus guide the combination therapy.

### Adoptive Cell Transfer

In certain embodiments, the methods of enhancing an anti-tumor immune response are administered with adoptive cell transfer (ACT). As used herein, “ACT”, “adoptive cell therapy” and “adoptive cell transfer” may be used interchangeably. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 June; 24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

In certain embodiments, modified T cells are used for an adoptive cell therapy. In one example embodiment, a modified T cell that expresses an enzyme capable of cleaving poly-LacNAc on its cell surface is administered. Not being bound by a theory, the T cell will be resistant to increased glycosylation on the surface of a tumor. The enzyme may be selected from the group consisting of endo H, endo F2, endo F3, peptide:N-glycosidase F (PNGase F), endo D, O-glycosidase, endo-β-galactosidase, sialidase and O-sialoglycoprotease. Any method of expressing a protein on the cell surface may be used. For example, the enzyme can be fused to a short sequence encoding a lipid modification, such as myristoyl and paloutoyl that automatically targets to the plasma membrane (MyrPalm) (see, e.g., Zacharias D A, Violin J D. Newton A C, Tsien R Y. Partitioning of lipid-modified monomeric GFPs into membrane microdomains of live cells. Science. 2002:296(5569):913-916). Alternative methods for membrane-targeting of the enzyme include other protein modifications (e g. polybasic domains or isoprenylation) or fusing the enzyme to a transmembrane protein.

Aspects of the invention involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: MR1 (see, e.g., Crowther, et al., 2020, Genome-wide CRISPR-Cas9 screening reveals ubiquitous T cell cancer targeting via the monomorphic MHC class I-related protein MR1, Nature Immunology volume 21, pages 178-185), B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gplOO; BCR-ABL (breakpoint cluster region-Abelson); tyrosinase; New York esophageal squamous cell carcinoma 1 (NY-ESO-1); K-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (ICE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac(2-8)aNeu5Ac(2-3)bDGalp(1-4)bDGlcp(1-1)Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis(Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac(2-3)bDGalp(1-4)bDGlcp(1-1)Cer); TGS5; high molecular weight-melanoma-associated antigen (HMWMAA); o-acetyl-GD2 ganglioside (OAcGD2); Folate receptor alpha; Folate receptor beta; tumor endothelial marker 1 (TEMI/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1, -2, -3 or -4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2, -3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); L1CAM (L1 cell adhesion molecule); MC1R (melanocortin 1 receptor); Myosin/m (myosin mutated); MUM-1, -2, -3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukaemia/retinoic acid receptor a); PRAME (preferentially expressed antigen of melanoma); SAGE (sarcoma antigen); TEL/AML 1 (translocation Ets-family leukemia/acute myeloid leukemia 1); TPI/m (triosephosphate isomerase mutated); CD70; and any combination thereof.

In certain embodiments, an antigen to be targeted in adoptive cell therapy (such as particularly CAR or TCR T-cell therapy) of a disease (such as particularly of tumor or cancer) is a tumor-specific antigen (TSA).

In certain embodiments, an antigen to be targeted in adoptive cell therapy (such as particularly CAR or TCR T-cell therapy) of a disease (such as particularly of tumor or cancer) is a neoantigen.

In certain embodiments, an antigen to be targeted in adoptive cell therapy (such as particularly CAR or TCR T-cell therapy) of a disease (such as particularly of tumor or cancer) is a tumor-associated antigen (TAA).

In certain embodiments, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), and any combinations thereof.

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CML), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR a and B chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8α hinge domain and a CD8α transmembrane domain, to the transmembrane and intracellular signaling domains of either CD3ζ or FcRγ (scFv-CD33 or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3g-chain, CD97, GDI 1a-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon R1b), CD79a, CD79b, Fc gamma RIIa, DAP10, and DAP12. In certain preferred embodiments, the primary signaling domain comprises a functional signaling domain of CD3ζ or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3 chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM 006139 (sequence version 1, 2 or 3): IEVMYPPPYLDNEKSNGTIIHVKGKHLCPSPLFPGPSKPFWVLVVVGGVLACYSLLVT VAFIIFWVRSKRSRLLHSDYMNMTPRRPGPTRKHYQPYAPPRDFAAYRS)) (SEQ ID NO: 18). Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, co-stimulation may be orchestrated by expressing CARs in antigen-specific T cells, chosen so as to be activated and expanded following engagement of their native αβTCR, for example by antigen on professional antigen-presenting cells, with attendant co-stimulation. In addition, additional engineered receptors may be provided on the immunoresponsive cells, for example to improve targeting of a T-cell attack and/or minimize side effects

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-(molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-(molecule. The exact sequence of the CD28 molecule included in the FMC63-28Z CAR corresponded to Genbank identifier NM_006139; the sequence included all amino acids starting with the amino acid sequence IEVMYPPPY (SEQ ID NO: 19) and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY (SEQ ID NO: 19) and continuing all the way to the carboxy-terminus of the protein. The sequence is reproduced herein: IEVMYPPPYLDNEKSNGTIIHVKGKHLCPSPLFPGPSKPFWVLVVVGGVLACY SLLVTVAFIIFWVRSKRSRLLHSDYMNMTPRRPGPTRKHYQPYAPPRDFAAYRS (SEQ ID NO: 18). Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in WO2015187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signaling domains (CD28-CD32; 4-1BB-CD3ζ; CD27-CD3ζ; CD28-CD27-CD3ζ, 4-1BB-CD27-CD3ζ; CD27-4-1BB-CD3ζ; CD28-CD27-FcεR1 gamma chain; or CD28-FcεRI gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signaling domain as set forth in Table 1 of WO2015187528. Preferably, the antigen is CD19, more preferably the antigen-binding element is an anti-CD19 scFv, even more preferably the mouse or human anti-CD19 scFv as described in Example 1 of WO2015187528. In certain embodiments, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20(1):55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am. J. Pathol. 1995; 147: 1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-a and TCR-B) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLOS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3 and either CD28 or CD137. Viral vectors may for example include vectors based on HIV, SV40, EBV, HSV or BPV. In certain embodiments, inducible gene switches are used to regulate expression of a CAR or TCR (see, e.g., Chakravarti, Deboki et al. “Inducible Gene Switches with Memory in Human T Cells for Cellular Immunotherapy.” ACS synthetic biology vol. 8, 8 (2019): 1744-1754).

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with y-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

In certain embodiments, ACT includes co-transferring CD4+ Th1 cells and CD8+ CTLs to induce a synergistic antitumour response (see, e.g., Li et al., Adoptive cell therapy with CD4+ T helper 1 cells and CD8+ cytotoxic T cells enhances complete rejection of an established tumour, leading to generation of endogenous memory responses to non-targeted tumour epitopes. Clin Transl Immunology. 2017 October; 6(10): e160).

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4″T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells or population of cells, may be made resistant to at least one immunosuppressive agent due to the inactivation of a gene encoding a receptor for such immunosuppressive agent. In certain embodiments, the immunosuppressive treatment provides for the selection and expansion of the immunoresponsive T cells within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e. intracavity delivery) or directly into a tumor prior to resection (i.e. intratumoral delivery). In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf”adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23(9):2255-2266. doi: 10.1158/1078-0432.CCR-16-1300. Epub 2016 Nov. 4; Qasim et al., 2017, Molecular remission of infant B-ALL after infusion of universal TALEN gene-edited CAR T cells, Sci Transl Med. 2017 Jan. 25; 9(374); Legut, et al., 2018, CRISPR-mediated TCR replacement generates superior anticancer transgenic T cells. Blood, 131(3), 311-322; Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018; and Roth, T. L. Editing of Endogenous Genes in Cellular Immunotherapies. Curr Hematol Malig Rep 15, 235-240 (2020)). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see PCT Patent Publications: WO2013176915, WO2014059173, WO2014172606, WO2014184744, and WO2014191128).

In certain embodiments, editing may result in inactivation of a gene. By inactivating a gene, it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells (such as by CRISPR/Cas), particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, a and B, which assemble to form a heterodimer and associates with the CD3-transducing subunits to form the T cell receptor complex present on the cell surface. Each a and B chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells (such as by CRISPR/Cas), particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells (such as by CRISPR/Cas), particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1) (see, e.g., Rupp L J, Schumann K, Roybal K T, et al. CRISPR/Cas9-mediated PD-1 disruption enhances anti-tumor efficacy of human chimeric antigen receptor T cells. Sci Rep. 2017; 7(1):737). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, WO2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as CRISPR, TALEN or ZFN) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, such as by CRISPR, ZNF or TALEN (for example, as described in WO201704916).

In certain embodiments, editing of cells (such as by CRISPR/Cas), particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in WO2016011210 and WO2017011804).

In certain embodiments, editing of cells (such as by CRISPR/Cas), particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In certain embodiments, a cell may be multiply edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20: 1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. Nos. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MHC class I may be evaluated indirectly by monitoring the ability to promote incorporation of 1251 labeled β2-microglobulin (B2m) into MHC class I/B2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™ BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000-fold, or most preferably at least about 100,000-fold. The numbers of T cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR, may be manufactured as described in WO2015120096, by a method comprising: enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO2015120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in WO2017070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in WO2016191756 comprising administering to the patient a dose of cyclophosphamide between 200 mg/m2/day and 2000 mg/m2/day and a dose of fludarabine between 20 mg/m²/day and 900 mg/m²/day.

In certain embodiments, a patient in need of adoptive cell transfer may be administered a TLR agonist to enhance anti-tumor immunity (see, e.g., Urban-Wojciuk, et al., The Role of TLRs in Anti-cancer Immunity and Tumor Rejection, Front Immunol. 2019; 10: 2388; and Kaczanowska et al., TLR agonists: our best frenemy in cancer immunotherapy, J Leukoc Biol. 2013 June; 93(6): 847-863). In certain embodiments, TLR agonists are delivered in a nanoparticle system (see, e.g., Buss and Bhatia, Nanoparticle delivery of immunostimulatory oligonucleotides enhances response to checkpoint inhibitor therapeutics, Proc Natl Acad Sci USA. 2020 Jun. 3; 202001569). In certain embodiments, the agonist is a TLR9 agonist. Id.

In certain embodiments, the adoptive cell transfer method comprises the administration of CAR (chimeric antigen receptor) T cells or natural killer cells, T cells expressing a T cell receptor (TCR) specific for tumor antigen, or tumor infiltrating lymphocytes (TILs).

### Checkpoint Blockade Therapy

In embodiments, the immunotherapy is checkpoint blockade (CPB) therapy. Antibodies that block the activity of checkpoint receptors, including CTLA-4, PD-1, Tim-3, Lag-3, and TIGIT, either alone or in combination, have been associated with improved effector CD8+ T cell responses in multiple pre-clinical cancer models (Johnston et al., 2014. The immunoreceptor TIGIT regulates antitumor and antiviral CD8(+) T cell effector function. Cancer cell 26, 923-937; Ngiow et al., 2011. Anti-TIM3 antibody promotes T cell IFN-gamma-mediated antitumor immunity and suppresses established tumors. Cancer research 71, 3540-3551; Sakuishi et al., 2010. Targeting Tim-3 and PD-1 pathways to reverse T cell exhaustion and restore anti-tumor immunity. The Journal of experimental medicine 207, 2187-2194; and Woo et al., 2012. Immune inhibitory molecules LAG-3 and PD-1 synergistically regulate T-cell function to promote tumoral immune escape. Cancer research 72, 917-927). Similarly, blockade of CTLA-4 and PD-1 in patients (Brahmer et al., 2012. Safety and activity of anti-PD-L1 antibody in patients with advanced cancer. The New England journal of medicine 366, 2455-2465; Hodi et al., 2010. Improved survival with ipilimumab in patients with metastatic melanoma. The New England journal of medicine 363, 711-723; Schadendorf et al., 2015. Pooled Analysis of Long-Term Survival Data From Phase II and Phase III Trials of Ipilimumab in Unresectable or Metastatic Melanoma. Journal of clinical oncology: official journal of the American Society of Clinical Oncology 33, 1889-1894; Topalian et al., 2012. Safety, activity, and immune correlates of anti-PD-1 antibody in cancer. The New England journal of medicine 366, 2443-2454; and Wolchok et al., 2017. Overall Survival with Combined Nivolumab and Ipilimumab in Advanced Melanoma. The New England journal of medicine 377, 1345-1356) has shown increased frequencies of proliferating T cells, often with specificity for tumor antigens, as well as increased CD8⁺ T cell effector function (Ayers et al., 2017. IFN-gamma-related mRNA profile predicts clinical response to PD-1 blockade. The Journal of clinical investigation 127, 2930-2940; Das et al., 2015. Combination therapy with anti-CTLA-4 and anti-PD-1 leads to distinct immunologic changes in vivo. Journal of immunology 194, 950-959; Gubin et al., 2014. Checkpoint blockade cancer immunotherapy targets tumour-specific mutant antigens. Nature 515, 577-581; Huang et al., 2017. T-cell invigoration to tumour burden ratio associated with anti-PD-1 response. Nature 545, 60-65; Kamphorst et al., 2017. Proliferation of PD-1+CD8 T cells in peripheral blood after PD-1-targeted therapy in lung cancer patients. Proceedings of the National Academy of Sciences of the United States of America 114, 4993-4998; Kvistborg et al., 2014. Anti-CTLA-4 therapy broadens the melanoma-reactive CD8+ T cell response. Science translational medicine 6, 254ra128; van Rooij et al., 2013. Tumor exome analysis reveals neoantigen-specific T-cell reactivity in an ipilimumab-responsive melanoma. Journal of clinical oncology: official journal of the American Society of Clinical Oncology 31, e439-442; and Yuan et al., 2008. CTLA-4 blockade enhances polyfunctional NY-ESO-1 specific T cell responses in metastatic melanoma patients with clinical benefit. Proceedings of the National Academy of Sciences of the United States of America 105, 20410-20415). Accordingly, the success of checkpoint receptor blockade has been attributed to the binding of blocking antibodies to checkpoint receptors expressed on dysfunctional CD8⁺ T cells and restoring effector function in these cells. The check point blockade therapy may be an inhibitor of any check point protein described herein. The checkpoint blockade therapy may comprise anti-TIM3, anti-CTLA4, anti-PD-L1, anti-PD1, anti-TIGIT, anti-LAG3, or combinations thereof. Anti-PD1 antibodies are disclosed in U.S. Pat. No. 8,735,553. Antibodies to LAG-3 are disclosed in U.S. Pat. No. 9,132,281. Anti-CTLA4 antibodies are disclosed in U.S. Pat. Nos. 9,327,014; 9,320,811; and 9,062,111. Specific check point inhibitors include, but are not limited to, anti-CTLA4 antibodies (e.g., Ipilimumab and Tremelimumab), anti-PD-1 antibodies (e.g., Nivolumab, Pembrolizumab), and anti-PD-L1 antibodies (e.g., Atezolizumab).

In certain embodiments, checkpoint inhibition may be enhanced by administering a TLR agonist to enhance anti-tumor immunity (see, e.g., Urban-Wojciuk, et al., The Role of TLRs in Anti-cancer Immunity and Tumor Rejection, Front Immunol. 2019; 10: 2388; and Kaczanowska et al., TLR agonists: our best frenemy in cancer immunotherapy, J Leukoc Biol. 2013 June; 93(6): 847-863). In certain embodiments, a TLR9 agonist is administered (see, e.g., Chuang, et al., Adjuvant Effect of Toll-Like Receptor 9 Activation on Cancer Immunotherapy Using Checkpoint Blockade, Front. Immunol., 29 May 2020; and Reilley, et al., TLR9 activation cooperates with T cell checkpoint blockade to regress poorly immunogenic melanoma, J. Immunotherapy Cancer, 2019, 7, 323). In certain embodiments, TLR agonists are delivered in a nanoparticle system (see, e.g., Buss and Bhatia, Nanoparticle delivery of immunostimulatory oligonucleotides enhances response to checkpoint inhibitor therapeutics, Proc Natl Acad Sci USA. 2020 Jun. 3; 202001569).

### Recombinant Protein Formulation, Dosage, and Delivery

In one example embodiment, virus like particles (VLPs) are used to facilitate intracellular recombinant protein therapy (see, e.g., WO2020252455A1, U.S. Ser. No. 10/577,397B2). In certain embodiments, VLPs include a Gag-B3GNT2, a Gag-MCL1, a Gag-BCL2A1, or a Gag-JUNB fusion protein. The Gag-B3GNT2, Gag-MCL1, Gag-BCL2A1, or Gag-JUNB fusion proteins may include a matrix protein, a capsid protein, and/or a nucleocapsid protein covalently linked to B3GNT2, MCL1, BCL2A1 or JUNB. In certain embodiments, the VLPs include a membrane comprising a phospholipid bilayer with one or more human endogenous retrovirus (HERV) derived ENV/glycoprotein(s) on the external side; a HERV-derived GAG protein in the VLP core, and a Gag-B3GNT2, a Gag-MCL1, a Gag-BCL2A1, or a Gag-JUNB fusion protein on the inside of the membrane, wherein B3GNT2, MCL1, BCL2A1 OR JUNB is fused to a human-endogenous GAG or other plasma membrane recruitment domain (see, e.g., WO2020252455A1). Fusion proteins can be obtained using standard recombinant protein technology.

In one example embodiment, cell-penetrating peptides (CPPs) are used to facilitate intracellular recombinant protein therapy (see, e.g., Dinca A, Chien W-M, Chin M T. Intracellular Delivery of Proteins with Cell-Penetrating Peptides for Therapeutic Uses in Human Disease. International Journal of Molecular Sciences. 2016; 17(2):263). In certain embodiments, cell-penetrating peptides can be conjugated to B3GNT2, MCL1, BCL2A1 or JUNB, for example, using standard recombinant protein technology. In certain embodiments, cell-penetrating peptides can be concurrently delivered with recombinant B3GNT2, MCL1, BCL2A1 or JUNB.

In one example embodiment, nanocarriers are used to facilitate intracellular recombinant protein therapy (see, e.g., Lee Y W, Luther D C, Kretzmann J A, Burden A, Jeon T, Zhai S, Rotello V M. Protein Delivery into the Cell Cytosol using Non-Viral Nanocarriers. Theranostics 2019; 9(11):3280-3292). Non-limiting nanocarriers include, but are not limited to nanoparticles (e.g., silica, gold), polymers, lipid based (e.g., cationic lipid within a polymer shell, lipid-like nanoparticles).

The pharmaceutical composition of the invention may be administered locally or systemically. In a preferred embodiment, the pharmaceutical composition is administered near the tissue whose cells are to be transduced. In a particular embodiment, the pharmaceutical composition of the invention is administered locally to the subcutaneous adipose tissue, which is composed of varying amounts of the two different types of adipose tissue: white adipose tissue (WAT) that stores energy in the form of triacylglycerol (TAG) and brown adipose tissue (BAT) that dissipates energy as heat, “burning” fatty acids to maintain body temperature. In one example embodiment, the pharmaceutical composition of the invention is administered in the white adipose tissue (WAT) and/or in the brown adipose tissue (BAT) by intra-WAT or intra-BAT injection. In another preferred embodiment, the pharmaceutical composition of the invention is administered systemically.

The “adeno-associated virus” (AAV) can be formulated with a physiologically acceptable carrier for use in gene transfer and gene therapy applications. The dosage of the formulation can be measured or calculated as viral particles or as genome copies (“GC”)/viral genomes (“vg”). Any method known in the art can be used to determine the genome copy (GC) number of the viral compositions of the invention. One method for performing AAV GC number titration is as follows: purified AAV vector samples are first treated with DNase to eliminate un-encapsulated AAV genome DNA or contaminating plasmid DNA from the production process. The DNase resistant particles are then subjected to heat treatment to release the genome from the capsid. The released genomes are then quantitated by real-time PCR using primer/probe sets targeting specific region of the viral genome.

In any of the described methods the one or more vectors may be comprised in a delivery system. In any of the described methods the vectors may be delivered via liposomes, particles (e.g. nanoparticles), exosomes, microvesicles, a gene-gun. In any of the described methods viral vectors may be delivered by transduction of viral particles. The delivery systems may be administered systemically or by localized administration (e.g., direct injection). The term “systemically administered” and “systemic administration”, as used herein, means that the polynucleotides, vectors, polypeptides, or pharmaceutical compositions of the invention are administered to a subject in a non-localized manner. The systemic administration of the polynucleotides, vectors, polypeptides, or pharmaceutical compositions of the invention may reach several organs or tissues throughout the body of the subject or may reach specific organs or tissues of the subject. For example, the intravenous administration of a pharmaceutical composition of the invention may result in the transduction of more than one tissue or organ in a subject. The term “transduce” or “transduction”, as used herein, refers to the process whereby a foreign nucleotide sequence is introduced into a cell via a viral vector. The term “transfection”, as used herein, refers to the introduction of DNA into a recipient eukaryotic cell.

Recombinant protein compositions described herein may be administered systemically (e.g., intravenously) or administered locally to adipose tissue (e.g., injection). In preferred embodiments, the recombinant protein compositions are administered with an appropriate carrier to be administered to a mammal, especially a human, preferably a pharmaceutically acceptable composition. A “pharmaceutically acceptable composition” refers to a non-toxic semisolid, liquid, or aerosolized filler, diluent, encapsulating material, colloidal suspension or formulation auxiliary of any type. Preferably, this composition is suitable for injection. These may be in particular isotonic, sterile, saline solutions (monosodium or disodium phosphate, sodium, potassium, calcium or magnesium chloride and similar solutions or mixtures of such salts), or dry, especially freeze-dried compositions which upon addition, depending on the case, of sterilized water or physiological saline, permit the constitution of injectable solutions.

### Alternate Tumor Immune Evasion Targets

Applicants identified additional gene targets that were enriched in the overexpression screens. Applicants used MAGeCK (Li et al., 2014) and FDR analyses to identify candidate genes that were enriched in cells cultured with ESO T cells relative to control (FIG. 1h-k, FIG. 2b, Tables 1A-B, 2, and 3A-B). Pathway analysis performed on 576 genes prioritized by the MAGeCK analysis, which included the top 1% of multiple screening replicates by combining the acute and chronic screens, revealed pathways that were significantly enriched (FDR<0.05) within these top 1% of candidates, including some that have been previously shown to be important for tumor immune evasion, such as lipopolysaccharide response, extrinsic apoptosis signaling, NF-κB activation, JAK-STAT signaling, and antigen presentation (Lawson et al., 2020; Manguso et al., 2017; Pan et al., 2018; Patel et al., 2017; Vredevoogd et al., 2019). This analysis identified the novel immune evasion targets, B3GNT2, MCL1, BCL2A1 and JUNB. Validation studies were performed for these genes and knockout of all genes resulted in enhanced vulnerability to an immunotherapy. A similar approach can be used to test and identify further novel targets for enhancing anti-tumor immunity. In certain embodiments, targets are validated in a tumor animal model. In certain embodiments, the genes are knocked out in a tumor model and tested for resistance and sensitivity to T cell killing. In certain embodiments, the genes are over expressed in a tumor model and tested for resistance and sensitivity to T cell killing. In certain embodiments, the alternative targets are used to enhance anti-tumor immunity. In preferred embodiments, the targets used are one or more of the top ten ranked genes in either Table 1 or Table 3.

The additional targets in Table 1 and Table 3 may also be targeted using the same approaches laid out above for the four targets B3GNT2, MCL1, BCLA21, and JUNB. In one example embodiment, any of the aforementioned methods targeting B3GNT2, MCL1, BCLA21, and JUNB, may further comprise targeting one or more genes in Table 1 or Table 3.

TABLE 1A
MAGeCK analysis results of the CRISPRa screen for resistance to T cell cytotoxicity
in the acute screen. Gene targets are in ranked order. Gene names, RefSeq IDs,
and MAGeCK P-values for each of the top 1000 screening bioreps are listed.

| | | Acute no T | Acute T | Acute no T | Acute T |
|---|---|---|---|---|---|
| Gene | id | cells biorep 1 | cells biorep 1 | cells biorep 2 | cells biorep 2 |
| CD274 | NM_014143 | 7.89E−04 | 2.08E−03 | 2.54E−03 | 8.41E−05 |
| MCL1 | NM_021960 | 4.57E−05 | 2.48E−04 | 3.23E−03 | 8.84E−03 |
| ZBTB10 | NM_001277145 | 2.52E−03 | 7.80E−03 | 2.18E−03 | 3.47E−03 |
| LMNA | NM_001257374 | 5.71E−03 | 1.29E−02 | 1.93E−04 | 5.54E−04 |
| C1R | NM_001733 | 4.34E−03 | 2.65E−03 | 1.35E−02 | 4.30E−03 |
| RABGGTA | NM_182836 | 8.06E−04 | 5.54E−04 | 6.64E−03 | 1.79E−02 |
| GNAL | NM_001142339 | 1.67E−02 | 9.79E−03 | 1.83E−03 | 1.06E−03 |
| KCNQ2 | NM_004518 | 4.98E−03 | 4.13E−03 | 3.06E−03 | 2.42E−02 |
| COMMD4 | NM_017828 | 2.54E−02 | 7.70E−03 | 3.55E−03 | 7.36E−03 |
| WRAP53 | NM_001143991 | 1.97E−02 | 2.41E−02 | 5.02E−03 | 1.06E−03 |
| GRIP1 | NM_001178074 | 4.66E−03 | 1.80E−03 | 2.72E−02 | 1.63E−02 |
| SCMH1 | NM_001172222 | 1.40E−02 | 2.43E−02 | 1.71E−02 | 1.28E−02 |
| OLFM1 | NM_006334 | 6.79E−03 | 7.90E−03 | 4.73E−02 | 9.83E−03 |
| RAB27A | NM_183234 | 1.16E−02 | 2.69E−02 | 2.18E−02 | 1.18E−02 |
| MBD3L3 | NM_001164425 | 4.33E−04 | 7.52E−04 | 2.45E−02 | 4.76E−02 |
| HGD | NM_000187 | 3.65E−02 | 1.36E−02 | 1.87E−02 | 5.77E−03 |
| JMJD1C | NM_001282948 | 2.63E−02 | 2.71E−02 | 4.16E−03 | 1.98E−02 |
| NXNL1 | NM_138454 | 1.48E−02 | 2.48E−02 | 1.68E−02 | 2.21E−02 |
| RNF166 | NM_001171816 | 2.38E−02 | 2.89E−02 | 7.35E−03 | 2.14E−02 |
| ISLR | NM_201526 | 4.33E−02 | 9.40E−03 | 2.44E−02 | 4.93E−03 |
| NLRP2 | NM_001174081 | 1.39E−02 | 1.13E−02 | 7.85E−03 | 5.10E−02 |
| JADE3 | NM_001077445 | 1.38E−02 | 1.16E−02 | 5.30E−02 | 7.53E−03 |
| MAGED2 | NM_014599 | 3.03E−02 | 1.33E−02 | 3.49E−02 | 7.73E−03 |
| DUS4L | NM_181581 | 1.58E−02 | 2.10E−02 | 2.06E−02 | 2.92E−02 |
| BCL2L1 | NM_001191 | 1.97E−02 | 4.92E−02 | 1.67E−02 | 1.61E−03 |
| SYCE1 | NM_001143764 | 4.62E−03 | 1.94E−02 | 5.93E−02 | 9.74E−03 |
| C12orf73 | NM_001135570 | 2.26E−02 | 2.03E−02 | 2.55E−03 | 5.37E−02 |
| ST8SIA5 | NM_013305 | 5.83E−03 | 2.54E−02 | 5.33E−02 | 1.59E−02 |
| MYBL1 | NM_001144755 | 2.76E−02 | 6.64E−02 | 2.12E−04 | 6.68E−03 |
| CYB5D2 | NM_001254755 | 8.96E−03 | 5.72E−03 | 2.67E−02 | 6.32E−02 |
| CGREF1 | NM_001166241 | 8.24E−03 | 3.42E−02 | 6.81E−03 | 6.03E−02 |
| SEC22A | NM_012430 | 2.67E−02 | 1.52E−02 | 4.59E−02 | 2.39E−02 |
| PROSC | NM_007198 | 5.23E−02 | 3.47E−02 | 3.30E−03 | 2.15E−02 |
| AHR | NM_001621 | 5.75E−02 | 1.59E−02 | 1.98E−02 | 1.90E−02 |
| FLCN | NM_144997 | 2.57E−02 | 8.54E−02 | 9.28E−04 | 4.42E−04 |
| C7orf76 | NM_001201451 | 1.94E−02 | 8.94E−02 | 1.94E−03 | 5.98E−03 |
| RARRES3 | NM_004585 | 2.93E−02 | 4.10E−02 | 5.37E−02 | 3.87E−03 |
| SDC2 | NM_002998 | 3.77E−02 | 7.81E−03 | 7.64E−02 | 7.01E−03 |
| PNLIPRP1 | NM_006229 | 6.86E−04 | 1.31E−03 | 4.58E−02 | 8.21E−02 |
| R3HDM1 | NM_001282799 | 4.76E−03 | 9.13E−03 | 6.55E−02 | 5.47E−02 |
| TLE2 | NM_001144761 | 2.44E−02 | 1.11E−01 | 2.11E−04 | 2.19E−03 |
| CUZD1 | NM_022034 | 1.50E−02 | 6.46E−03 | 6.61E−02 | 5.16E−02 |
| HIST3H2BB | NM_175055 | 3.05E−02 | 6.94E−02 | 2.83E−02 | 1.78E−02 |
| ARMCX5 | NM_001168480 | 7.73E−02 | 5.83E−02 | 6.70E−03 | 6.20E−03 |
| RPS7 | NM_001011 | 2.02E−02 | 2.28E−03 | 2.35E−02 | 1.09E−01 |
| PANK1 | NM_138316 | 4.77E−03 | 2.92E−02 | 1.03E−02 | 1.11E−01 |
| RABEPK | NM_001174153 | 6.36E−02 | 8.33E−02 | 2.50E−03 | 7.06E−03 |
| SNCA | NM_000345 | 4.28E−02 | 2.70E−02 | 6.66E−02 | 2.03E−02 |
| STAG2 | NM_001042749 | 1.32E−02 | 3.91E−02 | 3.59E−02 | 7.11E−02 |
| TYMS | NM_001071 | 4.00E−02 | 1.72E−02 | 6.79E−02 | 3.58E−02 |
| OR4F29 | NM_001005221 | 3.81E−02 | 8.66E−02 | 7.24E−03 | 3.55E−02 |
| KIAA1958 | NM_133465 | 3.90E−02 | 1.98E−02 | 8.09E−02 | 2.85E−02 |
| INTS10 | NM_018142 | 1.58E−03 | 1.63E−03 | 5.73E−02 | 1.08E−01 |
| CXorf57 | NM_018015 | 6.22E−02 | 9.19E−02 | 8.61E−03 | 6.65E−03 |
| DEFA3 | NM_005217 | 4.14E−02 | 2.56E−02 | 5.29E−02 | 5.05E−02 |
| PRKAG1 | NM_002733 | 8.95E−03 | 6.46E−02 | 6.73E−02 | 2.99E−02 |
| DSC1 | NM_004948 | 4.81E−03 | 1.69E−02 | 5.10E−02 | 9.85E−02 |
| JUNB | NM_002229 | 2.36E−05 | 2.30E−06 | 1.53E−01 | 1.85E−02 |
| RECK | NM_021111 | 2.66E−02 | 1.18E−01 | 2.06E−02 | 8.31E−03 |
| MBD3L2 | NM_144614 | 3.61E−03 | 1.39E−03 | 3.29E−02 | 1.36E−01 |
| PCNA | NM_182649 | 1.05E−02 | 4.13E−02 | 2.69E−02 | 9.57E−02 |
| MGAT5 | NM_002410 | 3.18E−02 | 2.21E−02 | 9.13E−02 | 3.03E−02 |
| UBA3 | NM_003968 | 3.12E−02 | 6.00E−02 | 5.92E−02 | 2.77E−02 |
| AMER3 | NM_001105193 | 2.33E−02 | 1.43E−01 | 6.64E−03 | 8.27E−03 |
| LAMC3 | NM_006059 | 4.70E−02 | 7.01E−02 | 2.21E−02 | 4.24E−02 |
| GSTCD | NM_024751 | 3.47E−02 | 3.71E−02 | 9.06E−02 | 1.99E−02 |
| ACVRL1 | NM_001077401 | 1.62E−02 | 3.10E−02 | 2.60E−02 | 1.10E−01 |
| REG4 | NM_001159352 | 1.26E−01 | 4.49E−02 | 2.71E−04 | 1.46E−02 |
| ZNF750 | NM_024702 | 2.41E−02 | 3.94E−02 | 1.03E−01 | 2.09E−02 |
| CDK16 | NM_006201 | 2.18E−02 | 7.07E−02 | 3.78E−02 | 5.78E−02 |
| GPR161 | NM_001267614 | 5.45E−02 | 2.03E−02 | 7.53E−02 | 3.81E−02 |
| COLEC11 | NM_001255988 | 6.91E−02 | 2.47E−02 | 6.03E−02 | 3.53E−02 |
| TBC1D9B | NM_015043 | 1.85E−02 | 6.46E−02 | 6.26E−02 | 4.44E−02 |
| C17orf105 | NM_001136483 | 2.28E−02 | 7.64E−02 | 6.88E−02 | 2.33E−02 |
| TMEM19 | NM_018279 | 6.92E−02 | 1.93E−02 | 5.18E−03 | 9.76E−02 |
| TRIM58 | NM_015431 | 1.09E−02 | 1.11E−01 | 1.03E−02 | 6.06E−02 |
| RASA2 | NM_006506 | 1.26E−01 | 2.61E−02 | 2.31E−02 | 1.76E−02 |
| RPRM | NM_019845 | 1.63E−02 | 1.55E−02 | 2.57E−02 | 1.40E−01 |
| TMEM201 | NM_001010866 | 4.18E−03 | 9.52E−03 | 1.47E−01 | 3.74E−02 |
| PTRH2 | NM_016077 | 6.69E−02 | 3.04E−02 | 9.66E−02 | 5.56E−03 |
| C11orf70 | NM_001195005 | 9.52E−02 | 6.53E−03 | 8.36E−02 | 1.50E−02 |
| TYK2 | NM_003331 | 1.39E−02 | 2.17E−02 | 6.36E−02 | 1.06E−01 |
| EPHA1 | NM_005232 | 4.17E−02 | 4.60E−02 | 1.86E−02 | 9.93E−02 |
| HIST1H2BI | NM_003525 | 2.52E−02 | 2.16E−02 | 8.00E−02 | 7.89E−02 |
| MFSD12 | NM_174983 | 4.09E−02 | 9.61E−03 | 7.30E−02 | 8.31E−02 |
| TAF1 | NM_001286074 | 3.90E−02 | 4.67E−02 | 1.91E−02 | 1.03E−01 |
| KIF21A | NM_001173463 | 4.10E−02 | 7.17E−02 | 8.08E−02 | 1.53E−02 |
| PRKACB | NM_002731 | 1.27E−01 | 8.23E−02 | 1.08E−03 | 2.93E−04 |
| GPT2 | NM_001142466 | 1.94E−02 | 3.63E−02 | 6.58E−02 | 8.94E−02 |
| CNP | NM_033133 | 3.06E−02 | 7.91E−03 | 7.74E−02 | 9.67E−02 |
| STARD13 | NM_178006 | 1.17E−01 | 7.94E−02 | 9.75E−03 | 6.71E−03 |
| SH2D1B | NM_053282 | 7.33E−02 | 3.68E−02 | 6.57E−02 | 3.76E−02 |
| TRHDE | NM_013381 | 9.76E−02 | 9.39E−02 | 1.14E−02 | 1.20E−02 |
| DCST2 | NM_144622 | 9.47E−02 | 3.29E−02 | 7.61E−02 | 1.49E−02 |
| KCTD18 | NM_152387 | 3.32E−02 | 3.66E−02 | 1.25E−01 | 2.41E−02 |
| CAPG | NM_001256139 | 5.11E−02 | 6.33E−02 | 9.26E−02 | 1.26E−02 |
| FOXC1 | NM_001453 | 7.38E−02 | 2.96E−02 | 8.31E−02 | 3.61E−02 |
| ZSCAN26 | NM_152736 | 1.66E−02 | 1.78E−02 | 9.68E−02 | 9.18E−02 |
| TMX1 | NM_030755 | 1.06E−03 | 8.06E−04 | 8.59E−02 | 1.36E−01 |
| GMIP | NM_001288998 | 3.65E−02 | 2.84E−02 | 4.01E−02 | 1.18E−01 |
| FAM65C | NM_080829 | 6.28E−03 | 2.62E−02 | 1.05E−01 | 8.63E−02 |
| TEX11 | NM_031276 | 1.03E−02 | 1.86E−02 | 1.10E−01 | 8.53E−02 |
| WDR35 | NM_020779 | 6.58E−02 | 7.19E−02 | 7.91E−03 | 8.03E−02 |
| BCAR1 | NM_001170719 | 5.94E−02 | 2.89E−02 | 5.87E−02 | 7.99E−02 |
| ANKRD66 | NM_001162435 | 4.25E−02 | 2.94E−02 | 7.04E−02 | 8.91E−02 |
| NGLY1 | NM_001145294 | 4.38E−02 | 3.28E−02 | 5.44E−02 | 1.02E−01 |
| UBE2NL | NM_001012989 | 2.08E−02 | 3.34E−02 | 4.93E−02 | 1.31E−01 |
| CYP1A2 | NM_000761 | 5.43E−02 | 1.26E−01 | 1.10E−02 | 4.42E−02 |
| TAAR2 | NM_014626 | 3.75E−02 | 1.62E−02 | 7.81E−02 | 1.04E−01 |
| ZMYM4 | NM_005095 | 5.95E−03 | 4.31E−03 | 6.38E−02 | 1.61E−01 |
| RPS3A | NM_001006 | 6.72E−03 | 1.46E−02 | 3.90E−02 | 1.77E−01 |
| FKBP2 | NM_004470 | 9.13E−02 | 4.84E−02 | 4.66E−02 | 5.09E−02 |
| ARNT | NM_001668 | 1.09E−01 | 1.25E−01 | 4.54E−03 | 3.73E−04 |
| UGT2B15 | NM_001076 | 3.73E−02 | 1.02E−01 | 1.53E−02 | 8.70E−02 |
| KCNRG | NM_199464 | 8.41E−02 | 4.65E−02 | 4.72E−02 | 6.37E−02 |
| CXCL5 | NM_002994 | 1.18E−02 | 3.26E−03 | 1.19E−01 | 1.08E−01 |
| ZBTB25 | NM_006977 | 1.31E−01 | 5.58E−02 | 2.26E−02 | 3.35E−02 |
| ZNF281 | NM_001281294 | 1.14E−02 | 9.46E−03 | 1.48E−01 | 7.41E−02 |
| PCDHA12 | NM_018903 | 4.78E−03 | 5.25E−03 | 1.04E−01 | 1.31E−01 |
| CDO1 | NM_001801 | 4.22E−02 | 1.03E−03 | 1.31E−01 | 7.15E−02 |
| BIN3 | NM_018688 | 8.58E−02 | 1.39E−01 | 1.70E−03 | 2.08E−02 |
| RIN2 | NM_001242581 | 5.01E−02 | 1.31E−01 | 2.22E−02 | 4.51E−02 |
| STK4 | NM_006282 | 1.02E−01 | 5.90E−02 | 5.53E−02 | 3.19E−02 |
| MYOM1 | NM_019856 | 5.36E−02 | 3.12E−02 | 7.01E−02 | 9.56E−02 |
| SMAD4 | NM_005359 | 1.16E−01 | 9.56E−02 | 7.10E−03 | 3.49E−02 |
| BBS5 | NM_152384 | 1.60E−02 | 2.11E−02 | 1.54E−01 | 6.34E−02 |
| NACC1 | NM_052876 | 1.63E−01 | 6.55E−02 | 4.54E−03 | 2.18E−02 |
| MITD1 | NM_138798 | 1.91E−02 | 3.05E−02 | 1.76E−01 | 2.91E−02 |
| BTN3A2 | NM_001197249 | 9.53E−02 | 1.21E−01 | 3.61E−02 | 3.31E−03 |
| NMU | NM_006681 | 3.72E−02 | 5.47E−02 | 1.28E−01 | 3.61E−02 |
| GABARAPL2 | NM_007285 | 6.80E−02 | 5.88E−02 | 1.08E−01 | 2.28E−02 |
| OLIG1 | NM_138983 | 7.40E−02 | 1.49E−01 | 1.51E−02 | 2.10E−02 |
| TBX21 | NM_013351 | 1.05E−01 | 2.54E−02 | 5.20E−02 | 7.78E−02 |
| SATB2 | NM_015265 | 4.86E−02 | 6.23E−02 | 9.44E−02 | 5.48E−02 |
| FGR | NM_001042747 | 9.96E−02 | 1.07E−01 | 3.48E−02 | 2.13E−02 |
| DCTN4 | NM_001135643 | 2.52E−02 | 8.57E−02 | 1.06E−01 | 4.70E−02 |
| C1QTNF9B-AS1 | NM_001014442 | 4.69E−02 | 6.80E−02 | 8.45E−02 | 6.60E−02 |
| CYP2S1 | NM_030622 | 2.39E−02 | 9.48E−03 | 1.73E−01 | 6.04E−02 |
| SLC1A1 | NM_004170 | 5.20E−02 | 1.44E−01 | 2.25E−02 | 4.97E−02 |
| CCDC88A | NM_018084 | 5.21E−02 | 2.04E−02 | 4.02E−02 | 1.56E−01 |
| C2orf88 | NM_001042521 | 3.74E−03 | 3.21E−03 | 8.03E−02 | 1.83E−01 |
| IL34 | NM_001172772 | 2.05E−02 | 2.31E−02 | 3.24E−02 | 1.95E−01 |
| COL11A1 | NM_001190709 | 4.14E−02 | 3.90E−02 | 1.69E−01 | 2.16E−02 |
| TMEM87B | NM_032824 | 3.66E−02 | 1.02E−01 | 6.13E−02 | 7.16E−02 |
| EBPL | NM_001278636 | 3.91E−02 | 1.34E−01 | 7.57E−02 | 2.22E−02 |
| SEPTIN9 | NM_001113495 | 7.89E−02 | 1.11E−01 | 4.30E−02 | 3.97E−02 |
| TSPY1 | NM_001197242 | 1.87E−01 | 3.84E−02 | 1.62E−02 | 3.40E−02 |
| RREB1 | NM_001003699 | 2.09E−02 | 6.98E−02 | 5.12E−02 | 1.34E−01 |
| AMMECR1L | NM_031445 | 3.07E−02 | 1.86E−01 | 4.65E−02 | 1.35E−02 |
| C1orf228 | NM_001145636 | 8.16E−02 | 3.45E−02 | 4.50E−02 | 1.16E−01 |
| MYL4 | NM_001002841 | 8.17E−02 | 1.02E−01 | 2.84E−02 | 6.50E−02 |
| REL | NM_002908 | 1.70E−01 | 5.86E−02 | 2.52E−02 | 2.48E−02 |
| ZNF675 | NM_138330 | 9.47E−02 | 6.40E−02 | 6.72E−02 | 5.50E−02 |
| KLRC1 | NM_002259 | 2.46E−03 | 9.19E−03 | 1.80E−01 | 9.05E−02 |
| ANO3 | NM_031418 | 6.47E−02 | 3.23E−02 | 2.54E−02 | 1.60E−01 |
| ACOT6 | NM_001037162 | 6.20E−02 | 2.20E−01 | 4.42E−04 | 1.55E−04 |
| FLNA | NM_001110556 | 6.08E−02 | 2.28E−02 | 7.12E−02 | 1.28E−01 |
| C9orf50 | NM_199350 | 7.60E−03 | 9.24E−03 | 1.09E−01 | 1.58E−01 |
| NLGN4Y | NM_001206850 | 2.64E−02 | 3.12E−02 | 1.33E−01 | 9.36E−02 |
| RPLP1 | NM_213725 | 1.04E−03 | 1.87E−03 | 5.50E−02 | 2.27E−01 |
| TBC1D2 | NM_001267572 | 1.25E−02 | 5.76E−02 | 5.68E−02 | 1.58E−01 |
| PAMR1 | NM_001001991 | 1.13E−01 | 1.15E−01 | 2.35E−02 | 3.33E−02 |
| NOTCH1 | NM_017617 | 4.13E−02 | 8.10E−02 | 3.00E−02 | 1.35E−01 |
| MAGEA11 | NM_005366 | 9.25E−02 | 8.37E−02 | 8.01E−02 | 3.28E−02 |
| AMZ1 | NM_001284355 | 7.75E−02 | 7.83E−02 | 2.41E−02 | 1.12E−01 |
| GPHN | NM_020806 | 3.04E−02 | 2.24E−02 | 5.78E−02 | 1.82E−01 |
| SOCS2 | NM_001270468 | 2.40E−03 | 3.16E−02 | 1.17E−01 | 1.42E−01 |
| HIST1H2BM | NM_003521 | 1.08E−01 | 6.06E−02 | 8.24E−03 | 1.17E−01 |
| KRT31 | NM_002277 | 1.67E−01 | 1.13E−01 | 8.49E−03 | 6.75E−03 |
| SECISBP2 | NM_001282690 | 1.53E−02 | 6.14E−02 | 1.89E−02 | 2.00E−01 |
| SMPDL3B | NM_001009568 | 2.73E−02 | 1.92E−03 | 3.43E−02 | 2.32E−01 |
| ARHGAP27 | NM_001282290 | 7.66E−03 | 2.57E−02 | 1.44E−01 | 1.18E−01 |
| PM20D2 | NM_001010853 | 1.36E−03 | 6.28E−02 | 4.19E−02 | 1.90E−01 |
| ZFP62 | NM_152283 | 8.20E−02 | 1.48E−01 | 2.31E−02 | 4.35E−02 |
| BAMBI | NM_012342 | 3.97E−02 | 1.97E−02 | 1.33E−01 | 1.05E−01 |
| CSTF1 | NM_001033522 | 3.02E−02 | 6.93E−02 | 1.52E−01 | 4.72E−02 |
| TRAF3 | NM_001199427 | 9.13E−02 | 9.01E−02 | 5.21E−02 | 6.54E−02 |
| ZNF385B | NM_001113398 | 1.55E−01 | 1.20E−01 | 8.72E−03 | 1.64E−02 |
| FAM64A | NM_001195228 | 1.08E−01 | 1.22E−01 | 4.43E−02 | 2.72E−02 |
| NOL4 | NM_001198549 | 6.84E−02 | 5.52E−02 | 1.31E−01 | 4.77E−02 |
| PSMC1 | NM_002802 | 3.06E−02 | 6.26E−02 | 6.19E−02 | 1.47E−01 |
| FAM53C | NM_001135647 | 5.38E−02 | 6.09E−02 | 4.52E−02 | 1.43E−01 |
| CSTB | NM_000100 | 1.98E−01 | 5.98E−02 | 3.53E−02 | 1.06E−02 |
| AKNA | NM_030767 | 7.16E−03 | 1.23E−02 | 1.76E−01 | 1.09E−01 |
| NGFR | NM_002507 | 6.21E−02 | 9.96E−02 | 1.92E−02 | 1.23E−01 |
| FAM175A | NM_139076 | 2.68E−02 | 1.24E−02 | 5.43E−02 | 2.11E−01 |
| DNMT3B | NM_175850 | 3.17E−02 | 6.16E−02 | 1.07E−01 | 1.06E−01 |
| P2RX5 | NM_001204519 | 1.23E−01 | 1.77E−01 | 1.11E−03 | 6.18E−03 |
| KCNQ5 | NM_001160130 | 7.71E−02 | 7.18E−02 | 9.38E−02 | 6.44E−02 |
| FRAT2 | NM_012083 | 1.78E−03 | 7.13E−04 | 1.06E−01 | 1.99E−01 |
| KRTAP21-2 | NM_181617 | 1.24E−01 | 1.09E−01 | 3.33E−02 | 4.25E−02 |
| PDC | NM_022576 | 8.59E−03 | 3.56E−02 | 2.14E−02 | 2.44E−01 |
| UBAC2 | NM_001144072 | 5.37E−02 | 3.48E−02 | 1.29E−01 | 9.47E−02 |
| ZNF548 | NM_001172773 | 4.05E−02 | 1.12E−02 | 1.06E−01 | 1.55E−01 |
| SPARCL1 | NM_001128310 | 6.02E−03 | 3.15E−02 | 1.60E−01 | 1.16E−01 |
| TTC38 | NM_017931 | 6.77E−02 | 9.54E−02 | 6.70E−02 | 8.38E−02 |
| TBX20 | NM_001077653 | 7.80E−02 | 2.97E−02 | 5.74E−02 | 1.49E−01 |
| CLEC19A | NM_001256720 | 1.24E−02 | 8.38E−03 | 2.34E−02 | 2.70E−01 |
| RBFOX1 | NM_001142333 | 1.01E−01 | 5.64E−02 | 9.43E−02 | 6.27E−02 |
| PCGF5 | NM_032373 | 5.17E−02 | 1.35E−01 | 3.99E−02 | 8.77E−02 |
| STEAP2 | NM_001040666 | 4.54E−03 | 2.75E−02 | 8.22E−02 | 2.00E−01 |
| OTOP1 | NM_177998 | 1.75E−01 | 4.05E−02 | 1.55E−02 | 8.40E−02 |
| DPP6 | NM_001039350 | 2.70E−02 | 9.94E−03 | 2.29E−02 | 2.56E−01 |
| FADD | NM_003824 | 1.13E−01 | 2.91E−02 | 7.55E−02 | 9.79E−02 |
| MOBP | NM_182935 | 9.97E−03 | 1.30E−02 | 8.45E−02 | 2.08E−01 |
| RPS27A | NM_001135592 | 1.90E−01 | 1.17E−01 | 4.65E−03 | 3.94E−03 |
| MAFF | NM_001161573 | 4.37E−02 | 9.33E−02 | 8.31E−02 | 9.60E−02 |
| CLCA2 | NM_006536 | 1.00E−01 | 1.11E−01 | 1.84E−02 | 8.71E−02 |
| CHMP1A | NM_001083314 | 8.71E−04 | 2.15E−02 | 1.53E−01 | 1.42E−01 |
| CLIC6 | NM_053277 | 2.15E−02 | 1.08E−01 | 1.75E−01 | 1.38E−02 |
| PHF1 | NM_002636 | 3.68E−02 | 8.08E−02 | 8.58E−02 | 1.16E−01 |
| ORAI3 | NM_152288 | 3.11E−02 | 3.95E−02 | 1.71E−01 | 7.84E−02 |
| CDKN1A | NM_001220778 | 5.97E−02 | 4.70E−02 | 5.95E−02 | 1.54E−01 |
| LSMEM2 | NM_153215 | 1.45E−01 | 7.51E−02 | 6.55E−02 | 3.44E−02 |
| CBL | NM_005188 | 7.66E−02 | 1.78E−01 | 3.08E−02 | 3.53E−02 |
| KCNK6 | NM_004823 | 4.21E−02 | 2.12E−02 | 9.08E−02 | 1.67E−01 |
| BCL6 | NM_001706 | 8.26E−02 | 6.28E−02 | 1.15E−02 | 1.64E−01 |
| KRTAP10-2 | NM_198693 | 4.34E−02 | 8.20E−02 | 4.48E−02 | 1.53E−01 |
| MAP2K5 | NM_001206804 | 1.28E−02 | 8.59E−02 | 7.37E−02 | 1.53E−01 |
| SEPP1 | NM_005410 | 2.75E−02 | 4.19E−02 | 1.67E−01 | 8.92E−02 |
| CHCHD4 | NM_001098502 | 3.44E−02 | 6.38E−03 | 1.00E−01 | 1.85E−01 |
| SRBD1 | NM_018079 | 3.65E−02 | 1.23E−01 | 1.26E−01 | 4.03E−02 |
| CNTNAP3B | NM_001201380 | 1.36E−01 | 2.13E−02 | 3.77E−02 | 1.31E−01 |
| CIZ1 | NM_001131015 | 1.55E−01 | 9.64E−02 | 4.40E−02 | 3.15E−02 |
| COX7C | NM_001867 | 8.18E−02 | 2.46E−02 | 9.23E−02 | 1.28E−01 |
| PDF | NM_022341 | 8.26E−02 | 3.11E−02 | 3.78E−02 | 1.76E−01 |
| TECPR1 | NM_015395 | 8.29E−02 | 4.70E−02 | 9.81E−02 | 9.99E−02 |
| IGJ | NM_144646 | 5.75E−02 | 1.14E−01 | 5.57E−02 | 1.01E−01 |
| CCDC30 | NM_001080850 | 7.21E−02 | 1.87E−01 | 2.08E−02 | 4.87E−02 |
| ABCA2 | NM_212533 | 2.20E−02 | 4.81E−02 | 8.89E−02 | 1.70E−01 |
| AKR7A2 | NM_003689 | 1.13E−01 | 8.73E−02 | 1.24E−01 | 4.59E−03 |
| KLK1 | NM_002257 | 3.97E−02 | 9.12E−03 | 1.59E−01 | 1.21E−01 |
| ALS2CL | NM_001190707 | 6.30E−02 | 1.41E−01 | 2.78E−02 | 9.85E−02 |
| PRIM2 | NM_001282488 | 1.97E−01 | 8.87E−02 | 4.26E−02 | 1.98E−03 |
| PPIC | NM_000943 | 3.16E−02 | 4.53E−02 | 1.37E−01 | 1.19E−01 |
| EGR3 | NM_004430 | 1.39E−01 | 1.02E−01 | 1.98E−02 | 7.24E−02 |
| ANKRD35 | NM_001280799 | 1.57E−02 | 1.80E−02 | 1.58E−01 | 1.41E−01 |
| OR5K4 | NM_001005517 | 3.36E−02 | 1.86E−02 | 7.78E−02 | 2.04E−01 |
| DNAH14 | NM_144989 | 7.77E−02 | 5.69E−02 | 5.16E−02 | 1.48E−01 |
| ATF7IP | NM_018179 | 7.14E−02 | 8.19E−02 | 7.56E−02 | 1.07E−01 |
| ESRRB | NM_004452 | 9.83E−02 | 1.10E−01 | 5.54E−02 | 7.24E−02 |
| GNPDA2 | NM_138335 | 1.79E−01 | 1.73E−02 | 1.32E−01 | 8.34E−03 |
| DEFB127 | NM_139074 | 2.34E−01 | 1.03E−01 | 6.82E−05 | 1.21E−04 |
| ATAD3B | NM_031921 | 1.57E−02 | 3.27E−02 | 1.50E−01 | 1.38E−01 |
| MEIS2 | NM_172315 | 5.25E−03 | 3.35E−03 | 2.72E−01 | 5.71E−02 |
| STX1B | NM_052874 | 1.03E−01 | 1.14E−01 | 1.01E−01 | 2.06E−02 |
| CHD1L | NM_001256336 | 6.88E−02 | 1.76E−01 | 5.45E−02 | 3.93E−02 |
| SEBOX | NM_001080837 | 3.02E−02 | 4.55E−02 | 2.61E−01 | 2.63E−03 |
| ERMP1 | NM_024896 | 2.34E−02 | 1.23E−01 | 1.17E−01 | 7.78E−02 |
| MSH6 | NM_001281494 | 2.05E−02 | 4.91E−02 | 2.33E−01 | 3.88E−02 |
| BTBD7 | NM_001289133 | 1.07E−01 | 1.30E−01 | 2.28E−02 | 8.30E−02 |
| PPP1R1B | NM_032192 | 6.45E−03 | 1.66E−02 | 2.64E−01 | 5.55E−02 |
| CELF2 | NM_001083591 | 4.41E−02 | 1.38E−01 | 5.05E−02 | 1.10E−01 |
| TMEM213 | NM_001085429 | 8.18E−02 | 1.08E−01 | 7.33E−02 | 7.98E−02 |
| USP5 | NM_003481 | 4.39E−02 | 6.29E−02 | 6.39E−02 | 1.73E−01 |
| HLA-DMA | NM_006120 | 3.72E−03 | 2.57E−02 | 1.83E−01 | 1.31E−01 |
| SNX20 | NM_001144972 | 6.43E−02 | 1.30E−01 | 5.19E−02 | 9.82E−02 |
| ZNF395 | NM_018660 | 1.58E−01 | 1.25E−02 | 1.69E−01 | 3.83E−03 |
| CENPE | NM_001813 | 2.94E−02 | 8.34E−02 | 1.64E−01 | 6.80E−02 |
| C18orf8 | NM_001276342 | 1.91E−01 | 7.06E−02 | 5.00E−02 | 3.39E−02 |
| HOXC13 | NM_017410 | 2.64E−02 | 2.07E−02 | 9.91E−02 | 2.01E−01 |
| ASB2 | NM_001202429 | 7.13E−02 | 4.24E−02 | 1.71E−01 | 6.29E−02 |
| SEPTIN1 | NM_052838 | 3.86E−02 | 1.90E−01 | 9.99E−02 | 1.94E−02 |
| WDR74 | NM_018093 | 3.46E−02 | 4.60E−02 | 7.92E−02 | 1.88E−01 |
| ZNF573 | NM_152360 | 5.01E−02 | 2.72E−02 | 1.01E−01 | 1.71E−01 |
| SLC25A24 | NM_013386 | 1.47E−01 | 1.45E−01 | 1.85E−02 | 3.80E−02 |
| LTA4H | NM_001256643 | 1.41E−01 | 7.55E−02 | 8.67E−02 | 4.64E−02 |
| DIRAS2 | NM_017594 | 1.30E−01 | 3.25E−02 | 2.22E−02 | 1.67E−01 |
| GJB6 | NM_006783 | 2.66E−02 | 2.67E−02 | 2.06E−01 | 9.15E−02 |
| EMP1 | NM_001423 | 6.28E−02 | 7.40E−02 | 1.26E−02 | 2.02E−01 |
| TES | NM_152829 | 1.60E−01 | 9.27E−02 | 1.65E−02 | 8.19E−02 |
| CPXCR1 | NM_033048 | 4.21E−02 | 5.08E−02 | 5.12E−02 | 2.09E−01 |
| C6orf58 | NM_001010905 | 2.04E−02 | 2.77E−02 | 2.51E−01 | 5.35E−02 |
| HDAC9 | NM_178425 | 4.46E−02 | 9.25E−02 | 4.93E−02 | 1.68E−01 |
| TRAPPC11 | NM_021942 | 3.67E−02 | 8.02E−03 | 1.58E−01 | 1.52E−01 |
| RNLS | NM_018363 | 2.78E−03 | 2.11E−03 | 3.26E−01 | 2.45E−02 |
| MS4A2 | NM_001256916 | 1.89E−01 | 1.16E−01 | 2.20E−02 | 3.02E−02 |
| COL9A3 | NM_001853 | 1.69E−01 | 1.31E−01 | 1.10E−02 | 4.72E−02 |
| HTR6 | NM_000871 | 9.08E−02 | 1.08E−01 | 1.02E−01 | 5.72E−02 |
| CATSPERB | NM_024764 | 1.42E−01 | 1.60E−01 | 3.62E−02 | 1.97E−02 |
| HYAL4 | NM_012269 | 5.77E−02 | 5.48E−02 | 5.79E−02 | 1.88E−01 |
| MTHFD2 | NM_006636 | 3.49E−02 | 1.89E−01 | 3.58E−02 | 9.94E−02 |
| NLRC4 | NM_021209 | 6.56E−02 | 1.54E−01 | 8.21E−02 | 5.82E−02 |
| ANXA8 | NM_001271702 | 1.27E−01 | 2.18E−01 | 1.10E−02 | 4.69E−03 |
| SPATS2L | NM_001282735 | 1.73E−03 | 3.54E−02 | 8.06E−02 | 2.43E−01 |
| MAP3K13 | NM_001242317 | 6.32E−02 | 1.19E−01 | 9.16E−02 | 8.74E−02 |
| SFMBT2 | NM_001018039 | 1.33E−01 | 2.11E−01 | 1.21E−02 | 5.29E−03 |
| LHX9 | NM_020204 | 7.80E−02 | 5.45E−02 | 1.15E−01 | 1.14E−01 |
| RSLID1 | NM_015659 | 1.11E−02 | 1.84E−02 | 1.54E−01 | 1.80E−01 |
| FBLIM1 | NM_001024216 | 3.68E−02 | 1.26E−01 | 6.47E−02 | 1.36E−01 |
| KRTAP13-2 | NM_181621 | 5.59E−02 | 1.71E−01 | 5.81E−02 | 7.85E−02 |
| NUS1 | NM_138459 | 1.40E−01 | 1.20E−01 | 3.62E−02 | 6.81E−02 |
| PRICKLE1 | NM_153026 | 9.74E−02 | 1.15E−01 | 1.14E−01 | 3.95E−02 |
| TEX33 | NM_001163857 | 1.39E−01 | 1.76E−02 | 1.32E−01 | 7.69E−02 |
| TMEM255B | NM_182614 | 1.23E−01 | 1.49E−01 | 4.74E−02 | 4.75E−02 |
| DEGS1 | NM_003676 | 1.76E−01 | 5.71E−02 | 8.08E−02 | 5.50E−02 |
| WDR33 | NM_018383 | 1.24E−01 | 1.83E−01 | 6.06E−02 | 1.37E−03 |
| RHOG | NM_001665 | 1.94E−01 | 1.39E−01 | 3.51E−02 | 2.52E−03 |
| LIN54 | NM_001115007 | 1.34E−01 | 6.20E−02 | 1.25E−01 | 4.87E−02 |
| STAM | NM_003473 | 1.35E−01 | 9.36E−02 | 8.32E−02 | 5.90E−02 |
| SPIN2B | NM_001006683 | 1.75E−02 | 1.56E−01 | 7.62E−02 | 1.21E−01 |
| RAD54B | NM_012415 | 1.24E−01 | 2.13E−01 | 1.20E−02 | 2.28E−02 |
| SDR39U1 | NM_001290292 | 1.93E−01 | 8.77E−02 | 8.97E−02 | 1.55E−03 |
| TAPT1 | NM_153365 | 3.71E−02 | 4.29E−02 | 6.67E−02 | 2.26E−01 |
| APC | NM_001127510 | 9.95E−02 | 1.62E−01 | 8.73E−03 | 1.03E−01 |
| RORC | NM_005060 | 2.20E−02 | 2.85E−01 | 3.50E−02 | 3.21E−02 |
| HMCES | NM_020187 | 1.46E−01 | 4.71E−02 | 1.06E−01 | 7.44E−02 |
| TCEAL2 | NM_080390 | 1.00E−01 | 1.32E−02 | 1.35E−01 | 1.26E−01 |
| AP5S1 | NM_018347 | 4.79E−02 | 8.25E−02 | 1.21E−01 | 1.22E−01 |
| SERINC1 | NM_020755 | 8.45E−02 | 1.34E−01 | 1.36E−02 | 1.43E−01 |
| LHX3 | NM_014564 | 1.52E−02 | 1.12E−02 | 2.54E−01 | 9.51E−02 |
| TMEM25 | NM_032780 | 7.76E−03 | 5.26E−03 | 2.73E−01 | 9.04E−02 |
| C8orf76 | NM_032847 | 2.31E−01 | 1.45E−01 | 7.51E−05 | 1.45E−04 |
| PIGS | NM_033198 | 7.00E−02 | 5.23E−02 | 1.13E−01 | 1.42E−01 |
| CHST8 | NM_001127895 | 1.50E−01 | 5.15E−02 | 1.19E−01 | 5.67E−02 |
| CHERP | NM_006387 | 9.40E−02 | 2.94E−02 | 1.12E−01 | 1.43E−01 |
| TMEM234 | NM_019118 | 6.45E−02 | 7.52E−02 | 1.43E−01 | 9.63E−02 |
| CYP3A43 | NM_057096 | 2.83E−02 | 2.00E−01 | 1.76E−02 | 1.35E−01 |
| CORO1B | NM_020441 | 2.14E−01 | 1.38E−01 | 7.96E−03 | 2.15E−02 |
| WBSCR16 | NM_148842 | 1.26E−01 | 1.74E−01 | 5.39E−02 | 2.78E−02 |
| PTPRCAP | NM_005608 | 6.01E−02 | 6.64E−02 | 1.03E−01 | 1.54E−01 |
| FHDC1 | NM_033393 | 3.93E−02 | 7.92E−02 | 1.18E−01 | 1.47E−01 |
| PILRA | NM_178273 | 1.87E−01 | 1.20E−01 | 1.99E−02 | 5.83E−02 |
| ZNF583 | NM_152478 | 2.40E−01 | 1.17E−01 | 1.37E−02 | 1.45E−02 |
| IFT81 | NM_014055 | 6.10E−02 | 3.00E−01 | 1.66E−02 | 7.87E−03 |
| TCTE3 | NM_174910 | 4.91E−02 | 2.92E−01 | 1.96E−02 | 2.44E−02 |
| ACADVL | NM_001270447 | 1.76E−01 | 1.85E−01 | 9.63E−03 | 1.59E−02 |
| OSGIN2 | NM_004337 | 9.50E−02 | 2.11E−01 | 5.95E−02 | 2.04E−02 |
| SMC4 | NM_001288753 | 8.17E−03 | 1.42E−02 | 1.40E−01 | 2.23E−01 |
| PRELID2 | NM_138492 | 1.36E−01 | 1.98E−01 | 3.70E−02 | 1.62E−02 |
| KRT20 | NM_019010 | 2.61E−02 | 2.93E−02 | 1.32E−01 | 2.00E−01 |
| SRSF9 | NM_003769 | 2.57E−01 | 1.17E−01 | 1.08E−02 | 4.78E−03 |
| ITFG2 | NM_018463 | 1.18E−01 | 1.48E−01 | 1.07E−01 | 1.65E−02 |
| CASC1 | NM_001204102 | 8.71E−02 | 1.64E−01 | 7.65E−02 | 6.14E−02 |
| NKX2-1 | NM_001079668 | 3.04E−02 | 6.60E−02 | 1.86E−03 | 2.91E−01 |
| CD72 | NM_001782 | 6.36E−02 | 4.50E−02 | 8.90E−02 | 1.92E−01 |
| DDX3Y | NM_001122665 | 1.81E−01 | 1.03E−01 | 3.28E−02 | 7.30E−02 |
| SLC16A11 | NM_153357 | 1.83E−01 | 1.74E−01 | 1.31E−02 | 1.90E−02 |
| ACYP1 | NM_001107 | 7.38E−02 | 4.30E−02 | 1.65E−01 | 1.07E−01 |
| KRTCAP2 | NM_173852 | 6.96E−02 | 2.69E−02 | 1.00E−01 | 1.93E−01 |
| CNPY3 | NM_006586 | 1.86E−01 | 8.36E−02 | 3.65E−02 | 8.40E−02 |
| CABYR | NM_153770 | 4.97E−02 | 3.87E−02 | 1.64E−01 | 1.37E−01 |
| PREPL | NM_006036 | 1.04E−01 | 1.49E−01 | 8.63E−02 | 5.13E−02 |
| IQCJ-SCHIP1 | NM_001197113 | 6.57E−03 | 2.70E−02 | 8.32E−02 | 2.73E−01 |
| C6orf211 | NM_001286562 | 3.50E−02 | 8.65E−02 | 9.20E−02 | 1.78E−01 |
| LOC100129520 | NM_001195272 | 1.19E−01 | 8.83E−02 | 5.22E−02 | 1.32E−01 |
| SREK1IP1 | NM_173829 | 3.15E−02 | 5.42E−02 | 2.07E−01 | 1.00E−01 |
| NAV3 | NM_014903 | 3.69E−03 | 1.27E−02 | 8.54E−02 | 2.91E−01 |
| ARID1B | NM_020732 | 1.01E−01 | 1.56E−01 | 7.77E−02 | 5.74E−02 |
| PRDM10 | NM_199439 | 9.82E−02 | 1.28E−01 | 9.79E−02 | 6.88E−02 |
| TOR1A | NM_000113 | 8.78E−02 | 3.41E−02 | 1.16E−01 | 1.56E−01 |
| HES1 | NM_005524 | 3.95E−03 | 7.82E−03 | 8.38E−02 | 2.99E−01 |
| CHID1 | NM_001142675 | 7.52E−02 | 2.26E−01 | 6.35E−02 | 2.96E−02 |
| COX14 | NM_001257134 | 7.25E−04 | 1.26E−03 | 2.00E−01 | 1.93E−01 |
| F8 | NM_000132 | 1.93E−01 | 1.05E−01 | 8.60E−02 | 1.12E−02 |
| PQBP1 | NM_001167990 | 1.79E−01 | 1.81E−01 | 3.09E−02 | 5.08E−03 |
| TK2 | NM_001271934 | 2.35E−02 | 3.56E−02 | 1.46E−01 | 1.92E−01 |
| COMTD1 | NM_144589 | 4.83E−02 | 8.43E−02 | 5.92E−02 | 2.08E−01 |
| ALKBH8 | NM_138775 | 1.06E−01 | 4.79E−02 | 1.84E−01 | 6.22E−02 |
| XRCC6BP1 | NM_033276 | 3.81E−02 | 6.76E−02 | 2.41E−01 | 5.32E−02 |
| DEFA6 | NM_001926 | 5.30E−02 | 1.34E−02 | 1.31E−01 | 2.03E−01 |
| ETV3L | NM_001004341 | 1.44E−01 | 1.62E−01 | 5.06E−02 | 4.35E−02 |
| LNX2 | NM_153371 | 1.49E−01 | 6.34E−02 | 5.76E−02 | 1.31E−01 |
| MNAT1 | NM_002431 | 9.82E−02 | 1.02E−01 | 2.27E−02 | 1.78E−01 |
| CREM | NM_182724 | 1.19E−01 | 2.27E−01 | 1.01E−02 | 4.56E−02 |
| DTNA | NM_001198944 | 1.16E−01 | 1.05E−01 | 1.41E−01 | 3.88E−02 |
| GPR149 | NM_001038705 | 2.31E−01 | 1.43E−01 | 9.84E−03 | 1.77E−02 |
| GALK2 | NM_001001556 | 8.10E−03 | 2.79E−02 | 1.85E−01 | 1.81E−01 |
| COQ2 | NM_015697 | 5.74E−02 | 6.34E−02 | 1.52E−01 | 1.30E−01 |
| STAP2 | NM_017720 | 2.73E−02 | 2.58E−01 | 4.63E−02 | 7.20E−02 |
| CALHM1 | NM_001001412 | 1.11E−01 | 5.00E−02 | 8.20E−02 | 1.61E−01 |
| RXRB | NM_021976 | 5.92E−02 | 7.73E−02 | 1.62E−01 | 1.06E−01 |
| HIST1H4F | NM_003540 | 1.62E−01 | 2.23E−01 | 7.68E−03 | 1.27E−02 |
| PRDM13 | NM_021620 | 1.51E−02 | 6.09E−03 | 2.09E−01 | 1.77E−01 |
| PELO | NM_015946 | 5.39E−03 | 1.93E−02 | 1.52E−01 | 2.31E−01 |
| WIBG | NM_001143853 | 8.40E−02 | 1.36E−01 | 1.20E−01 | 6.84E−02 |
| UBE2J1 | NM_016021 | 1.60E−01 | 2.40E−01 | 5.92E−03 | 4.04E−03 |
| SDHB | NM_003000 | 1.31E−01 | 2.44E−01 | 3.14E−02 | 2.81E−03 |
| EIF1AD | NM_001242484 | 7.31E−02 | 1.38E−01 | 1.50E−01 | 5.04E−02 |
| DGCR6L | NM_033257 | 4.14E−02 | 1.09E−01 | 2.11E−01 | 5.07E−02 |
| SLC7A5 | NM_003486 | 1.88E−01 | 1.64E−01 | 4.73E−02 | 1.35E−02 |
| MYADM | NM_001020818 | 8.89E−02 | 5.87E−02 | 1.28E−01 | 1.37E−01 |
| CAMKK2 | NM_172226 | 8.65E−02 | 6.69E−02 | 2.53E−01 | 6.54E−03 |
| ZNF667 | NM_022103 | 1.77E−01 | 3.65E−02 | 1.46E−01 | 5.46E−02 |
| TRIM64C | NM_001206631 | 1.55E−01 | 7.12E−02 | 5.37E−02 | 1.34E−01 |
| ACP6 | NM_016361 | 1.77E−01 | 1.63E−01 | 6.70E−02 | 7.58E−03 |
| YOD1 | NM_018566 | 1.08E−01 | 7.92E−02 | 1.36E−01 | 9.10E−02 |
| TSHZ1 | NM_005786 | 2.32E−02 | 8.95E−02 | 1.10E−01 | 1.92E−01 |
| IDH2 | NM_001289910 | 3.37E−02 | 8.92E−02 | 1.63E−01 | 1.30E−01 |
| AKR1C1 | NM_001353 | 5.19E−02 | 3.96E−02 | 2.33E−01 | 9.34E−02 |
| MAML1 | NM_014757 | 1.12E−01 | 1.52E−02 | 1.60E−01 | 1.32E−01 |
| KRTAP26-1 | NM_203405 | 4.75E−03 | 1.85E−02 | 2.09E−01 | 1.86E−01 |
| IFT80 | NM_001190241 | 1.38E−01 | 1.89E−01 | 5.02E−02 | 4.16E−02 |
| MLPH | NM_001281473 | 1.13E−02 | 1.28E−02 | 3.12E−01 | 8.29E−02 |
| SEMA4G | NM_001203244 | 1.69E−02 | 9.93E−02 | 1.68E−01 | 1.35E−01 |
| VAT1L | NM_020927 | 2.09E−02 | 2.15E−02 | 2.66E−01 | 1.12E−01 |
| RAB41 | NM_001032726 | 1.20E−01 | 1.05E−01 | 1.21E−01 | 7.53E−02 |
| CELA2B | NM_015849 | 2.13E−04 | 1.57E−04 | 4.24E−02 | 3.79E−01 |
| H2AFY2 | NM_018649 | 8.86E−02 | 1.13E−01 | 1.74E−01 | 4.58E−02 |
| TMED2 | NM_006815 | 9.94E−02 | 9.17E−02 | 8.26E−02 | 1.49E−01 |
| PPFIBP2 | NM_003621 | 3.36E−02 | 4.55E−02 | 8.97E−02 | 2.54E−01 |
| ARHGAP29 | NM_004815 | 6.11E−02 | 1.15E−02 | 7.58E−02 | 2.75E−01 |
| MAGEE1 | NM_020932 | 1.54E−02 | 7.41E−02 | 1.96E−01 | 1.39E−01 |
| KDELC1 | NM_024089 | 8.49E−02 | 1.37E−01 | 1.68E−01 | 3.41E−02 |
| RING1 | NM_002931 | 2.89E−01 | 6.33E−02 | 1.86E−02 | 5.45E−02 |
| TMEM116 | NM_001193453 | 2.93E−01 | 7.19E−02 | 1.67E−02 | 4.43E−02 |
| MARVELD3 | NM_052858 | 2.98E−01 | 6.11E−02 | 4.37E−02 | 2.35E−02 |
| OR9K2 | NM_001005243 | 1.21E−01 | 1.14E−01 | 1.41E−01 | 5.11E−02 |
| TSEN34 | NM_001282333 | 2.61E−01 | 5.68E−02 | 8.67E−02 | 2.19E−02 |
| HAPLN4 | NM_023002 | 2.29E−01 | 9.75E−03 | 1.20E−01 | 6.79E−02 |
| HRASLS5 | NM_054108 | 1.20E−01 | 1.65E−01 | 4.46E−02 | 9.77E−02 |
| COX4I2 | NM_032609 | 1.86E−01 | 1.86E−01 | 1.30E−02 | 4.22E−02 |
| SLC45A3 | NM_033102 | 9.38E−02 | 6.36E−02 | 8.78E−02 | 1.83E−01 |
| AATF | NM_012138 | 7.46E−02 | 7.42E−02 | 9.78E−02 | 1.82E−01 |
| SMIM12 | NM_138428 | 5.62E−02 | 1.54E−01 | 1.73E−01 | 4.71E−02 |
| LPPR5 | NM_001037317 | 2.04E−01 | 1.52E−01 | 4.91E−02 | 2.53E−02 |
| SMIM13 | NM_001135575 | 9.51E−02 | 6.75E−02 | 5.76E−02 | 2.10E−01 |
| SLC39A1 | NM_001271958 | 1.67E−01 | 1.73E−01 | 2.02E−02 | 7.08E−02 |
| ELAC2 | NM_001165962 | 2.14E−01 | 1.32E−01 | 1.76E−02 | 6.90E−02 |
| LOC100131303 | NM_001282442 | 2.76E−03 | 2.52E−03 | 3.94E−02 | 3.89E−01 |
| SETD1A | NM_014712 | 6.80E−02 | 1.49E−01 | 1.65E−01 | 5.16E−02 |
| MASP2 | NM_006610 | 1.61E−01 | 9.03E−02 | 1.02E−01 | 8.00E−02 |
| BAHD1 | NM_014952 | 1.64E−01 | 1.25E−01 | 8.64E−02 | 5.91E−02 |
| CDK19 | NM_015076 | 1.71E−03 | 1.07E−03 | 3.24E−01 | 1.08E−01 |
| SLCO1B3 | NM_019844 | 1.54E−01 | 1.82E−01 | 2.38E−02 | 7.51E−02 |
| ITPRIPL2 | NM_001034841 | 1.25E−01 | 1.42E−01 | 1.41E−01 | 2.71E−02 |
| GATA1 | NM_002049 | 9.91E−02 | 1.94E−01 | 6.17E−02 | 8.06E−02 |
| FUT4 | NM_002033 | 3.13E−02 | 3.14E−02 | 1.98E−01 | 1.75E−01 |
| FAM156B | NM_001099684 | 1.81E−01 | 2.15E−01 | 1.71E−02 | 2.40E−02 |
| OR5H6 | NM_001005479 | 9.15E−03 | 3.04E−02 | 1.46E−01 | 2.52E−01 |
| MAGEC3 | NM_138702 | 5.90E−02 | 8.05E−02 | 1.75E−01 | 1.23E−01 |
| MAP3K3 | NM_203351 | 1.66E−01 | 2.40E−01 | 2.97E−03 | 2.93E−02 |
| IMP3 | NM_018285 | 1.57E−01 | 4.68E−02 | 9.37E−02 | 1.41E−01 |
| LMOD1 | NM_012134 | 1.97E−01 | 1.76E−01 | 2.55E−02 | 3.99E−02 |
| ARHGAP18 | NM_033515 | 8.10E−03 | 1.57E−02 | 2.63E−01 | 1.54E−01 |
| KCNIP4 | NM_001035004 | 3.44E−02 | 7.57E−02 | 1.10E−01 | 2.21E−01 |
| SKA1 | NM_145060 | 1.21E−01 | 1.89E−01 | 1.62E−02 | 1.15E−01 |
| METRN | NM_024042 | 2.12E−02 | 3.02E−03 | 3.50E−01 | 6.74E−02 |
| CEACAM1 | NM_001024912 | 5.36E−02 | 2.55E−02 | 9.43E−02 | 2.68E−01 |
| PKD2L2 | NM_001258449 | 1.14E−01 | 1.92E−01 | 5.68E−02 | 7.91E−02 |
| PUM2 | NM_001282791 | 6.90E−02 | 1.77E−01 | 2.95E−02 | 1.68E−01 |
| REPS1 | NM_001128617 | 5.53E−02 | 1.49E−01 | 1.53E−01 | 8.74E−02 |
| HIST1H4J | NM_021968 | 8.29E−02 | 6.05E−02 | 2.37E−01 | 6.51E−02 |
| LRRC29 | NM_001004055 | 2.15E−01 | 1.83E−01 | 1.39E−02 | 3.40E−02 |
| SMIM5 | NM_001162995 | 6.71E−02 | 7.41E−02 | 7.88E−02 | 2.26E−01 |
| STAT3 | NM_213662 | 7.42E−02 | 1.81E−01 | 6.86E−03 | 1.84E−01 |
| PBRM1 | NM_018313 | 1.66E−01 | 8.39E−02 | 2.56E−02 | 1.72E−01 |
| ISLR2 | NM_001130136 | 1.86E−01 | 5.17E−02 | 1.54E−01 | 5.58E−02 |
| PLEKHF1 | NM_024310 | 1.68E−01 | 3.23E−02 | 8.59E−02 | 1.61E−01 |
| C2orf50 | NM_182500 | 1.55E−01 | 1.28E−01 | 6.36E−02 | 1.01E−01 |
| INPP5A | NM_005539 | 4.07E−02 | 1.50E−01 | 2.90E−02 | 2.29E−01 |
| ELP5 | NM_015362 | 1.21E−01 | 1.72E−01 | 1.18E−01 | 3.77E−02 |
| DLK2 | NM_023932 | 1.29E−01 | 1.03E−01 | 8.37E−02 | 1.33E−01 |
| MTA2 | NM_004739 | 1.52E−01 | 7.42E−02 | 1.82E−01 | 4.19E−02 |
| SPTBN1 | NM_003128 | 6.26E−02 | 4.67E−03 | 2.77E−01 | 1.07E−01 |
| DEDD | NM_001039712 | 2.50E−01 | 1.32E−01 | 4.30E−02 | 2.79E−02 |
| PFKFB2 | NM_006212 | 2.41E−01 | 7.34E−02 | 1.08E−01 | 3.07E−02 |
| CPNE8 | NM_153634 | 1.96E−01 | 1.88E−01 | 4.78E−02 | 2.24E−02 |
| PRR35 | NM_145270 | 1.33E−01 | 2.08E−01 | 9.34E−02 | 1.86E−02 |
| PHF21A | NM_016621 | 2.13E−01 | 5.21E−02 | 7.38E−02 | 1.14E−01 |
| RANBP10 | NM_020850 | 1.86E−02 | 5.53E−02 | 1.40E−01 | 2.41E−01 |
| CCDC22 | NM_014008 | 1.47E−01 | 1.71E−01 | 7.13E−02 | 6.53E−02 |
| ISLR2 | NM_001130137 | 9.77E−02 | 4.76E−02 | 1.32E−01 | 1.78E−01 |
| STPG1 | NM_001199012 | 1.01E−01 | 2.51E−01 | 2.50E−02 | 7.85E−02 |
| TAL1 | NM_003189 | 8.62E−02 | 7.89E−02 | 1.24E−01 | 1.67E−01 |
| LINC01272 | NM_001278655 | 3.63E−02 | 3.98E−02 | 2.80E−01 | 9.98E−02 |
| N4BP2L2 | NM_014887 | 1.64E−02 | 3.59E−03 | 1.67E−01 | 2.70E−01 |
| BCAT1 | NM_001178093 | 1.62E−01 | 1.08E−01 | 1.80E−01 | 7.29E−03 |
| CNGB1 | NM_001297 | 4.32E−02 | 1.73E−01 | 7.47E−02 | 1.67E−01 |
| USP9X | NM_001039590 | 1.42E−01 | 1.20E−01 | 8.49E−02 | 1.12E−01 |
| VSTM2B | NM_001146339 | 5.85E−02 | 1.37E−01 | 1.06E−01 | 1.59E−01 |
| ABCC8 | NM_001287174 | 1.18E−03 | 1.06E−03 | 2.38E−01 | 2.19E−01 |
| OR2AG2 | NM_001004490 | 3.95E−02 | 7.97E−02 | 1.55E−01 | 1.86E−01 |
| COMMD1 | NM_152516 | 2.65E−02 | 1.92E−02 | 2.60E−01 | 1.55E−01 |
| TMCO2 | NM_001008740 | 3.68E−02 | 6.02E−02 | 2.21E−01 | 1.43E−01 |
| HOXA4 | NM_002141 | 1.29E−01 | 1.33E−01 | 7.57E−02 | 1.24E−01 |
| G0S2 | NM_015714 | 1.03E−01 | 2.93E−02 | 2.41E−01 | 8.80E−02 |
| KLF7 | NM_001270942 | 5.39E−02 | 1.87E−02 | 3.23E−01 | 6.61E−02 |
| PRAF2 | NM_007213 | 1.38E−01 | 1.92E−01 | 2.73E−02 | 1.04E−01 |
| TPP1 | NM_000391 | 2.22E−02 | 2.32E−02 | 9.17E−02 | 3.25E−01 |
| PIRT | NM_001101387 | 2.09E−01 | 1.53E−01 | 6.55E−02 | 3.58E−02 |
| CXCL1 | NM_001511 | 2.05E−01 | 1.96E−01 | 3.82E−02 | 2.47E−02 |
| PRSS1 | NM_002769 | 9.53E−02 | 1.49E−01 | 2.13E−01 | 7.41E−03 |
| ZFP28 | NM_020828 | 9.52E−03 | 5.37E−03 | 2.58E−01 | 1.92E−01 |
| CEP83 | NM_001042399 | 4.08E−02 | 6.87E−02 | 1.31E−01 | 2.24E−01 |
| NGDN | NM_015514 | 2.07E−01 | 5.99E−02 | 7.47E−02 | 1.23E−01 |
| SSX7 | NM_173358 | 1.27E−01 | 3.27E−01 | 2.77E−03 | 8.41E−03 |
| APBB1IP | NM_019043 | 1.41E−02 | 1.19E−02 | 1.53E−01 | 2.87E−01 |
| OR52D1 | NM_001005163 | 1.30E−01 | 3.01E−02 | 1.10E−01 | 1.96E−01 |
| GTF2F1 | NM_002096 | 6.08E−02 | 9.78E−02 | 6.81E−02 | 2.40E−01 |
| RPS27A | NM_001177413 | 1.63E−01 | 3.66E−02 | 2.27E−02 | 2.45E−01 |
| HSD17B1 | NM_000413 | 1.05E−01 | 1.31E−01 | 1.76E−01 | 5.69E−02 |
| TCF23 | NM_175769 | 2.78E−02 | 3.09E−02 | 3.99E−01 | 1.05E−02 |
| DHX33 | NM_001199699 | 1.72E−01 | 1.45E−01 | 1.14E−01 | 3.86E−02 |
| MARCO | NM_006770 | 8.32E−02 | 1.24E−01 | 1.99E−01 | 6.27E−02 |
| TAL2 | NM_005421 | 1.39E−02 | 1.10E−01 | 2.73E−01 | 7.28E−02 |
| SNN | NM_003498 | 1.13E−01 | 2.09E−01 | 9.52E−02 | 5.20E−02 |
| IMMP1L | NM_144981 | 1.18E−01 | 6.45E−02 | 3.00E−02 | 2.58E−01 |
| TDRD10 | NM_182499 | 2.01E−01 | 2.20E−01 | 2.94E−02 | 1.92E−02 |
| TBL1X | NM_001139467 | 4.08E−02 | 9.20E−02 | 2.07E−01 | 1.31E−01 |
| MGAT5B | NM_001199172 | 2.30E−01 | 2.30E−01 | 8.37E−03 | 2.40E−03 |
| MAFA | NM_201589 | 1.57E−01 | 1.74E−01 | 1.31E−02 | 1.26E−01 |
| PDXDC1 | NM_015027 | 3.77E−02 | 1.14E−01 | 1.28E−01 | 1.92E−01 |
| TBC1D4 | NM_014832 | 1.88E−01 | 8.43E−02 | 8.30E−02 | 1.17E−01 |
| ZBTB7B | NM_001252406 | 1.64E−01 | 1.31E−01 | 1.18E−01 | 5.90E−02 |
| FIGN | NM_018086 | 1.31E−01 | 1.13E−01 | 9.22E−02 | 1.36E−01 |
| LIPT1 | NM_145198 | 6.15E−02 | 8.32E−02 | 2.70E−01 | 5.81E−02 |
| TMC5 | NM_001261841 | 4.80E−02 | 4.18E−02 | 3.34E−02 | 3.50E−01 |
| FBXO42 | NM_018994 | 1.91E−01 | 1.94E−01 | 4.41E−02 | 4.43E−02 |
| ASZ1 | NM_130768 | 2.19E−02 | 4.94E−03 | 1.78E−01 | 2.69E−01 |
| HERPUD2 | NM_022373 | 2.72E−01 | 9.80E−02 | 9.97E−02 | 4.54E−03 |
| LGALS9C | NM_001040078 | 2.32E−02 | 4.06E−02 | 1.81E−01 | 2.30E−01 |
| SPTBN1 | NM_178313 | 3.12E−01 | 1.60E−01 | 2.07E−03 | 8.67E−04 |
| SLC35C1 | NM_018389 | 4.24E−02 | 6.17E−02 | 2.91E−01 | 8.15E−02 |
| UBASH3B | NM_032873 | 6.59E−02 | 2.48E−01 | 9.47E−02 | 6.84E−02 |
| CHST5 | NM_024533 | 8.46E−02 | 6.11E−02 | 2.45E−01 | 8.65E−02 |
| RNF121 | NM_018320 | 8.03E−02 | 3.64E−01 | 6.97E−03 | 2.54E−02 |
| IPO4 | NM_024658 | 1.83E−01 | 2.21E−01 | 5.25E−02 | 2.00E−02 |
| KLK7 | NM_005046 | 3.64E−01 | 9.49E−02 | 1.32E−02 | 5.39E−03 |
| VSX2 | NM_182894 | 1.53E−02 | 4.84E−03 | 2.91E−01 | 1.67E−01 |
| MDM2 | NM_002392 | 2.87E−02 | 6.55E−02 | 3.19E−02 | 3.54E−01 |
| GBP4 | NM_052941 | 2.78E−02 | 2.25E−02 | 2.93E−01 | 1.37E−01 |
| AMPD1 | NM_000036 | 6.89E−02 | 1.01E−01 | 1.54E−01 | 1.57E−01 |
| OR4C11 | NM_001004700 | 2.80E−01 | 1.60E−01 | 1.29E−02 | 2.82E−02 |
| CCDC104 | NM_001282761 | 7.44E−02 | 1.00E−01 | 2.80E−01 | 2.73E−02 |
| HTR2A | NM_001165947 | 1.77E−02 | 3.09E−01 | 6.41E−02 | 9.08E−02 |
| TDRD3 | NM_001146070 | 9.01E−02 | 3.09E−01 | 1.28E−02 | 7.04E−02 |
| SAMD3 | NM_001258275 | 4.95E−03 | 7.24E−03 | 5.48E−02 | 4.16E−01 |
| LIN7A | NM_004664 | 1.97E−01 | 6.13E−02 | 1.16E−01 | 1.09E−01 |
| C16orf74 | NM_206967 | 1.23E−01 | 3.19E−02 | 2.90E−01 | 3.78E−02 |
| UQCRHL | NM_001089591 | 6.74E−02 | 9.98E−02 | 1.44E−01 | 1.72E−01 |
| ACADVL | NM_000018 | 2.57E−02 | 6.48E−02 | 3.15E−01 | 7.76E−02 |
| GUCY1A3 | NM_001130684 | 1.47E−02 | 4.65E−02 | 2.14E−01 | 2.09E−01 |
| GLB1 | NM_001135602 | 1.43E−01 | 8.76E−02 | 1.34E−01 | 1.21E−01 |
| MAST3 | NM_015016 | 8.28E−02 | 2.04E−02 | 2.60E−01 | 1.22E−01 |
| ARMCX3 | NM_016607 | 7.74E−02 | 7.56E−02 | 1.43E−01 | 1.90E−01 |
| C1orf85 | NM_001256608 | 1.45E−01 | 3.13E−02 | 8.43E−02 | 2.25E−01 |
| MYO1B | NM_001161819 | 1.85E−02 | 3.80E−02 | 1.18E−01 | 3.11E−01 |
| CCDC7 | NM_145023 | 4.53E−02 | 3.60E−02 | 1.77E−01 | 2.27E−01 |
| DLG4 | NM_001365 | 1.57E−01 | 1.20E−01 | 6.42E−02 | 1.45E−01 |
| ATP6V1C1 | NM_001695 | 2.14E−01 | 2.37E−01 | 2.43E−02 | 1.02E−02 |
| FAM78A | NM_033387 | 2.25E−01 | 3.56E−02 | 1.11E−01 | 1.15E−01 |
| FOXN1 | NM_003593 | 1.34E−01 | 2.71E−01 | 4.32E−02 | 3.81E−02 |
| CDKN2A | NM_000077 | 2.72E−01 | 2.03E−01 | 8.10E−03 | 4.37E−03 |
| ELL2 | NM_012081 | 9.65E−02 | 2.12E−01 | 1.41E−01 | 3.71E−02 |
| RIIAD1 | NM_001144956 | 3.37E−01 | 1.47E−01 | 2.05E−03 | 1.92E−03 |
| PRKAG3 | NM_017431 | 7.90E−02 | 5.65E−02 | 1.82E−01 | 1.70E−01 |
| HPSE2 | NM_001166244 | 1.60E−01 | 2.51E−01 | 1.32E−02 | 6.30E−02 |
| OR51I1 | NM_001005288 | 2.22E−01 | 1.90E−01 | 7.70E−03 | 6.87E−02 |
| XRCC2 | NM_005431 | 2.33E−02 | 6.25E−02 | 1.98E−01 | 2.05E−01 |
| PPP2R2C | NM_001206996 | 8.06E−02 | 1.79E−01 | 7.40E−02 | 1.55E−01 |
| ATF6 | NM_007348 | 1.69E−01 | 2.66E−01 | 2.71E−02 | 2.67E−02 |
| ETV6 | NM_001987 | 9.22E−02 | 6.90E−02 | 9.05E−02 | 2.37E−01 |
| NAIP | NM_004536 | 1.54E−02 | 1.09E−01 | 2.35E−01 | 1.30E−01 |
| XAGE1E | NM_001097604 | 8.23E−02 | 2.00E−01 | 1.38E−01 | 6.86E−02 |
| IPP | NM_001145349 | 2.24E−01 | 2.38E−01 | 1.45E−02 | 1.30E−02 |
| DYRK1A | NM_001396 | 4.39E−02 | 5.43E−02 | 1.08E−01 | 2.84E−01 |
| DYRK4 | NM_003845 | 1.11E−01 | 1.15E−01 | 2.08E−01 | 5.58E−02 |
| SIN3A | NM_015477 | 2.53E−02 | 7.65E−02 | 1.22E−01 | 2.67E−01 |
| C10orf71 | NM_001135196 | 9.59E−02 | 2.66E−01 | 1.77E−02 | 1.11E−01 |
| MAP2K2 | NM_030662 | 1.88E−01 | 3.21E−02 | 1.69E−01 | 1.02E−01 |
| BMPR1B | NM_001256793 | 8.80E−02 | 1.11E−01 | 2.12E−01 | 8.10E−02 |
| BCL7B | NM_001707 | 9.86E−03 | 3.05E−02 | 9.44E−02 | 3.57E−01 |
| LMF1 | NM_022773 | 3.65E−03 | 1.15E−02 | 2.12E−01 | 2.65E−01 |
| DOCK8 | NM_203447 | 2.27E−01 | 2.53E−01 | 1.92E−03 | 1.03E−02 |
| ODF3L2 | NM_182577 | 1.12E−01 | 1.29E−01 | 1.62E−01 | 8.92E−02 |
| TRUB2 | NM_015679 | 1.80E−01 | 1.21E−01 | 6.26E−02 | 1.30E−01 |
| ACSL6 | NM_001205251 | 1.15E−01 | 2.69E−01 | 3.73E−02 | 7.29E−02 |
| PDE1B | NM_001165975 | 2.21E−01 | 1.55E−01 | 5.60E−02 | 6.23E−02 |
| SRPR | NM_001177842 | 4.94E−02 | 1.92E−01 | 9.23E−02 | 1.61E−01 |
| HIPK1 | NM_198269 | 1.52E−01 | 7.91E−02 | 2.12E−01 | 5.09E−02 |
| OR5D16 | NM_001005496 | 1.27E−01 | 4.56E−02 | 1.65E−01 | 1.58E−01 |
| BFSP1 | NM_001195 | 4.43E−02 | 6.44E−02 | 2.74E−01 | 1.13E−01 |
| MAPK10 | NM_002753 | 7.05E−02 | 1.58E−01 | 1.46E−01 | 1.21E−01 |
| CLEC12A | NM_201623 | 1.93E−02 | 7.76E−02 | 2.34E−01 | 1.66E−01 |
| DYNLRB1 | NM_014183 | 1.78E−01 | 1.63E−01 | 6.30E−02 | 9.34E−02 |
| COPZ1 | NM_001271735 | 9.47E−02 | 8.42E−02 | 1.88E−01 | 1.30E−01 |
| CTBP2 | NM_001083914 | 1.13E−02 | 3.18E−02 | 3.16E−01 | 1.38E−01 |
| GSG1 | NM_001206843 | 1.36E−01 | 1.42E−01 | 1.54E−01 | 6.58E−02 |
| SLC4A2 | NM_001199692 | 3.18E−01 | 1.64E−01 | 6.15E−03 | 8.35E−03 |
| HIST1H3A | NM_003529 | 1.38E−01 | 1.42E−01 | 7.20E−02 | 1.46E−01 |
| SLC7A1 | NM_003045 | 3.64E−02 | 1.45E−02 | 1.15E−01 | 3.33E−01 |
| MORC1 | NM_014429 | 7.29E−02 | 1.56E−01 | 1.55E−01 | 1.17E−01 |
| TLE3 | NM_020908 | 1.47E−01 | 7.60E−02 | 1.50E−02 | 2.62E−01 |
| LAMTOR2 | NM_001145264 | 4.50E−02 | 6.94E−02 | 1.46E−01 | 2.40E−01 |
| EIF3H | NM_003756 | 7.23E−02 | 7.78E−02 | 2.06E−01 | 1.45E−01 |
| SCD5 | NM_001037582 | 6.32E−02 | 7.72E−02 | 1.23E−01 | 2.38E−01 |
| ITPKA | NM_002220 | 6.38E−02 | 1.56E−01 | 1.33E−01 | 1.50E−01 |
| LIPH | NM_139248 | 4.89E−02 | 1.44E−01 | 1.98E−01 | 1.11E−01 |
| WDR18 | NM_024100 | 1.78E−01 | 1.30E−01 | 1.50E−01 | 4.54E−02 |
| WDR55 | NM_017706 | 1.92E−02 | 1.77E−02 | 2.72E−01 | 1.95E−01 |
| ALDH8A1 | NM_022568 | 1.28E−01 | 1.27E−01 | 4.81E−02 | 2.02E−01 |
| TMEM256 | NM_152766 | 1.92E−01 | 2.64E−01 | 3.55E−02 | 1.29E−02 |
| MYH10 | NM_001256095 | 1.64E−01 | 2.47E−01 | 5.18E−02 | 4.23E−02 |
| TERF2IP | NM_018975 | 8.64E−03 | 2.40E−03 | 4.29E−01 | 6.47E−02 |
| UBE2V2 | NM_003350 | 1.37E−01 | 1.12E−01 | 5.47E−02 | 2.02E−01 |
| SLC39A13 | NM_001128225 | 5.56E−02 | 5.96E−02 | 1.17E−01 | 2.73E−01 |
| MUC22 | NM_001198815 | 2.24E−02 | 1.47E−01 | 9.82E−02 | 2.38E−01 |
| SLC35F2 | NM_017515 | 1.42E−01 | 8.06E−02 | 2.33E−01 | 5.27E−02 |
| PHLPP2 | NM_001289003 | 5.70E−02 | 7.01E−02 | 1.05E−01 | 2.76E−01 |
| LTA | NM_001159740 | 9.85E−02 | 2.21E−01 | 1.12E−01 | 7.82E−02 |
| MATR3 | NM_001194954 | 4.23E−02 | 2.21E−02 | 3.12E−01 | 1.33E−01 |
| DDRGK1 | NM_023935 | 2.23E−02 | 1.30E−01 | 1.02E−01 | 2.55E−01 |
| THBD | NM_000361 | 6.09E−02 | 1.59E−01 | 2.77E−01 | 1.33E−02 |
| MYO15A | NM_016239 | 2.34E−01 | 5.88E−02 | 1.34E−02 | 2.04E−01 |
| MRPL55 | NM_181456 | 1.21E−01 | 1.65E−01 | 1.27E−01 | 9.69E−02 |
| OR5R1 | NM_001004744 | 7.03E−02 | 3.82E−01 | 1.37E−02 | 4.42E−02 |
| FBXL14 | NM_152441 | 8.95E−02 | 1.27E−01 | 2.16E−01 | 7.80E−02 |
| JAZF1 | NM_175061 | 3.84E−02 | 8.26E−02 | 1.73E−01 | 2.16E−01 |
| TRAPPC3 | NM_001270895 | 1.50E−01 | 5.39E−02 | 2.01E−01 | 1.06E−01 |
| MAPT | NM_005910 | 1.43E−01 | 1.61E−01 | 1.43E−01 | 6.30E−02 |
| FBXO32 | NM_148177 | 1.24E−01 | 2.97E−01 | 6.82E−02 | 2.20E−02 |
| FAIM | NM_001033032 | 2.14E−02 | 4.96E−02 | 3.17E−01 | 1.23E−01 |
| TMEM31 | NM_182541 | 4.30E−01 | 6.73E−02 | 1.41E−02 | 5.85E−04 |
| OR5AN1 | NM_001004729 | 6.30E−03 | 6.88E−03 | 3.90E−01 | 1.09E−01 |
| C1orf21 | NM_030806 | 2.99E−01 | 1.87E−01 | 2.55E−02 | 2.16E−03 |
| CKMT1A | NM_001015001 | 5.30E−02 | 1.47E−01 | 1.71E−01 | 1.43E−01 |
| TMEM11 | NM_003876 | 1.19E−01 | 4.73E−02 | 2.42E−01 | 1.06E−01 |
| TBX18 | NM_001080508 | 3.17E−02 | 9.74E−03 | 1.47E−01 | 3.26E−01 |
| THSD1 | NM_199263 | 2.56E−02 | 7.46E−03 | 2.42E−01 | 2.42E−01 |
| ZBTB18 | NM_001278196 | 2.58E−01 | 2.40E−01 | 6.35E−03 | 1.22E−02 |
| ANKRD26 | NM_014915 | 7.58E−02 | 3.80E−01 | 9.36E−03 | 5.30E−02 |
| TMED5 | NM_001167830 | 1.66E−01 | 2.37E−01 | 4.97E−02 | 6.60E−02 |
| PAIP2B | NM_020459 | 6.06E−02 | 2.20E−02 | 3.50E−01 | 8.73E−02 |
| CEP112 | NM_001037325 | 4.29E−02 | 1.45E−02 | 2.35E−01 | 2.27E−01 |
| MRPL23 | NM_021134 | 3.81E−02 | 2.99E−02 | 2.96E−01 | 1.56E−01 |
| UBA5 | NM_198329 | 1.96E−01 | 3.07E−01 | 5.97E−03 | 1.14E−02 |
| DYNLT3 | NM_006520 | 7.26E−02 | 7.41E−02 | 2.02E−01 | 1.72E−01 |
| PLG | NM_000301 | 3.43E−03 | 1.86E−02 | 3.39E−01 | 1.61E−01 |
| SLC12A6 | NM_001042497 | 7.80E−03 | 6.65E−03 | 2.11E−01 | 2.98E−01 |
| LPAR6 | NM_001162497 | 1.17E−01 | 1.40E−01 | 1.59E−01 | 1.07E−01 |
| OSR1 | NM_145260 | 1.02E−01 | 8.56E−02 | 6.14E−02 | 2.75E−01 |
| CLEC4E | NM_014358 | 1.90E−01 | 1.32E−01 | 5.64E−02 | 1.46E−01 |
| MED14 | NM_004229 | 1.55E−01 | 9.15E−02 | 2.53E−01 | 2.63E−02 |
| SLCO1B7 | NM_001009562 | 1.36E−01 | 1.19E−01 | 1.28E−01 | 1.43E−01 |
| MARCHF1 | NM_017923 | 3.11E−01 | 1.52E−01 | 5.65E−03 | 5.68E−02 |
| DAPK1 | NM_001288731 | 7.43E−02 | 7.38E−02 | 2.12E−01 | 1.66E−01 |
| CDK20 | NM_001039803 | 1.69E−01 | 2.36E−01 | 1.01E−01 | 2.03E−02 |
| ITM2C | NM_030926 | 1.60E−02 | 7.28E−03 | 2.00E−01 | 3.04E−01 |
| XAGE2 | NM_130777 | 1.90E−01 | 1.99E−01 | 9.83E−02 | 4.06E−02 |
| ROS1 | NM_002944 | 6.20E−02 | 5.57E−02 | 4.25E−02 | 3.68E−01 |
| IPO7 | NM_006391 | 8.25E−03 | 5.60E−03 | 3.39E−01 | 1.76E−01 |
| C20orf197 | NM_173644 | 1.76E−01 | 5.01E−02 | 1.24E−01 | 1.79E−01 |
| OPN5 | NM_181744 | 1.12E−01 | 1.20E−01 | 1.52E−01 | 1.48E−01 |
| SMAD1 | NM_005900 | 1.93E−01 | 1.79E−01 | 1.20E−01 | 3.91E−02 |
| PAK3 | NM_001128168 | 3.83E−02 | 6.82E−02 | 1.64E−01 | 2.61E−01 |
| CTRL | NM_001907 | 1.70E−01 | 2.02E−01 | 8.99E−02 | 6.85E−02 |
| PITX2 | NM_001204398 | 6.92E−02 | 3.39E−01 | 6.76E−02 | 5.58E−02 |
| MEF2C | NM_001193349 | 4.35E−02 | 5.50E−02 | 2.62E−02 | 4.07E−01 |
| G3BP1 | NM_198395 | 1.02E−01 | 2.06E−01 | 9.98E−02 | 1.25E−01 |
| SLC35A2 | NM_005660 | 3.60E−01 | 1.12E−01 | 1.31E−02 | 4.85E−02 |
| SPCS3 | NM_021928 | 1.98E−01 | 2.67E−02 | 1.16E−01 | 1.92E−01 |
| CCSER2 | NM_001284242 | 2.05E−01 | 3.18E−01 | 8.88E−03 | 1.44E−03 |
| SOS1 | NM_005633 | 5.78E−02 | 1.14E−01 | 7.40E−02 | 2.88E−01 |
| EIF2AK2 | NM_001135652 | 2.04E−02 | 4.42E−02 | 2.84E−01 | 1.85E−01 |
| SRP68 | NM_001260503 | 3.10E−02 | 2.06E−02 | 2.46E−01 | 2.37E−01 |
| TRIM68 | NM_018073 | 5.16E−02 | 1.29E−01 | 6.06E−02 | 2.93E−01 |
| GSTK1 | NM_001143680 | 2.39E−01 | 1.24E−01 | 1.43E−01 | 2.92E−02 |
| AKAP14 | NM_178813 | 2.21E−01 | 1.86E−01 | 2.92E−02 | 9.95E−02 |
| DNAH8 | NM_001206927 | 8.41E−02 | 2.81E−01 | 1.87E−02 | 1.52E−01 |
| CCDC88B | NM_032251 | 1.40E−01 | 1.36E−01 | 1.43E−01 | 1.18E−01 |
| PTPRR | NM_001207016 | 6.90E−02 | 5.20E−02 | 1.68E−01 | 2.48E−01 |
| SLC35F3 | NM_173508 | 4.47E−03 | 5.67E−03 | 3.27E−01 | 1.99E−01 |
| CD44 | NM_001202557 | 1.84E−01 | 5.87E−02 | 2.77E−01 | 1.73E−02 |
| LRRC36 | NM_001161575 | 2.97E−01 | 2.16E−01 | 2.06E−02 | 3.38E−03 |
| C1orf27 | NM_001164246 | 1.42E−02 | 3.07E−02 | 4.08E−01 | 8.49E−02 |
| ALKBH6 | NM_032878 | 7.27E−02 | 1.19E−01 | 3.44E−01 | 2.13E−03 |
| TLR10 | NM_030956 | 2.43E−01 | 2.38E−01 | 2.35E−02 | 3.36E−02 |
| C6orf47 | NM_021184 | 1.35E−01 | 1.19E−01 | 1.08E−01 | 1.76E−01 |
| SLFNL1 | NM_144990 | 3.31E−02 | 1.24E−01 | 1.26E−01 | 2.55E−01 |
| RASSF1 | NM_001206957 | 1.71E−01 | 5.61E−02 | 2.25E−01 | 8.72E−02 |
| BUB1B | NM_001211 | 1.90E−01 | 2.03E−01 | 1.03E−01 | 4.32E−02 |
| ADK | NM_001202449 | 1.30E−01 | 2.76E−02 | 1.88E−01 | 1.93E−01 |
| PSMD10 | NM_002814 | 1.05E−01 | 4.54E−02 | 1.54E−01 | 2.36E−01 |
| PSMB4 | NM_002796 | 2.83E−02 | 2.72E−02 | 1.95E−01 | 2.90E−01 |
| PWP1 | NM_007062 | 1.93E−04 | 4.42E−04 | 1.59E−01 | 3.81E−01 |
| EFCAB3 | NM_001144933 | 3.99E−02 | 2.98E−02 | 3.66E−01 | 1.06E−01 |
| AKT1 | NM_005163 | 1.76E−01 | 1.38E−01 | 8.49E−02 | 1.42E−01 |
| OR12D2 | NM_013936 | 1.41E−01 | 2.12E−01 | 2.48E−02 | 1.65E−01 |
| NAPSA | NM_004851 | 1.50E−01 | 2.87E−01 | 4.75E−02 | 5.79E−02 |
| TRAK1 | NM_001265609 | 2.45E−01 | 2.21E−01 | 4.50E−02 | 3.25E−02 |
| USP33 | NM_015017 | 2.57E−01 | 2.40E−01 | 1.20E−02 | 3.50E−02 |
| POU5F1 | NM_203289 | 1.72E−01 | 3.28E−01 | 1.22E−02 | 3.16E−02 |
| KRTAP19-8 | NM_001099219 | 4.64E−02 | 3.26E−02 | 1.31E−01 | 3.35E−01 |
| PLEKHG5 | NM_001042665 | 1.64E−02 | 9.96E−03 | 3.27E−01 | 1.92E−01 |
| SMPDL3A | NM_006714 | 1.92E−01 | 2.19E−01 | 6.63E−02 | 6.94E−02 |
| HIF3A | NM_ 152794 | 3.75E−03 | 1.12E−02 | 2.96E−01 | 2.35E−01 |
| RAB3IP | NM_022456 | 1.54E−01 | 2.27E−01 | 3.67E−02 | 1.30E−01 |
| HIST1H2BH | NM_003524 | 2.46E−02 | 4.05E−02 | 3.79E−01 | 1.04E−01 |
| FIGF | NM_004469 | 1.02E−01 | 1.91E−01 | 2.39E−01 | 1.58E−02 |
| CHD7 | NM_017780 | 1.68E−01 | 1.75E−01 | 7.75E−02 | 1.28E−01 |
| ZNF664-FAM101A | NM_001204299 | 3.10E−01 | 5.49E−02 | 1.65E−01 | 1.91E−02 |
| AASDHPPT | NM_015423 | 7.56E−02 | 7.13E−02 | 1.68E−01 | 2.34E−01 |
| AQP2 | NM_000486 | 2.60E−01 | 2.28E−01 | 5.97E−02 | 7.44E−04 |
| ZNF808 | NM_001039886 | 4.43E−02 | 3.79E−02 | 1.39E−01 | 3.28E−01 |
| VCX | NM_013452 | 1.04E−01 | 1.61E−01 | 2.35E−01 | 4.96E−02 |
| SLC4A8 | NM_001258403 | 2.28E−03 | 9.86E−03 | 3.26E−01 | 2.12E−01 |
| USP44 | NM_032147 | 1.36E−01 | 1.87E−01 | 1.22E−01 | 1.05E−01 |
| ACSS1 | NM_001252676 | 3.25E−02 | 2.20E−02 | 2.05E−01 | 2.92E−01 |
| AIFM2 | NM_032797 | 4.79E−02 | 3.78E−02 | 2.18E−01 | 2.48E−01 |
| KHDC1L | NM_001126063 | 4.45E−02 | 4.77E−02 | 4.11E−01 | 4.90E−02 |
| VN1R5 | NM_173858 | 1.18E−01 | 3.55E−02 | 1.68E−01 | 2.31E−01 |
| DNAJA4 | NM_001130183 | 3.57E−01 | 8.72E−02 | 9.57E−02 | 1.24E−02 |
| MID1 | NM_001193277 | 1.84E−01 | 1.56E−01 | 8.78E−02 | 1.25E−01 |
| PARP9 | NM_001146104 | 8.81E−02 | 4.84E−02 | 2.82E−01 | 1.35E−01 |
| DHTKD1 | NM_018706 | 9.40E−02 | 9.62E−02 | 6.68E−02 | 2.96E−01 |
| PIK3C2A | NM_002645 | 1.79E−01 | 1.69E−01 | 1.89E−01 | 1.62E−02 |
| RHPN2 | NM_033103 | 9.29E−02 | 2.99E−02 | 1.56E−01 | 2.75E−01 |
| FOCAD | NM_017794 | 1.08E−01 | 1.27E−01 | 2.62E−01 | 5.59E−02 |
| LRP10 | NM_014045 | 1.99E−01 | 2.20E−01 | 6.49E−02 | 6.94E−02 |
| FSD1 | NM_024333 | 2.50E−01 | 2.33E−01 | 6.58E−02 | 5.10E−03 |
| TRAP1 | NM_016292 | 2.02E−01 | 2.37E−01 | 3.23E−03 | 1.12E−01 |
| CD209 | NM_001144897 | 1.84E−01 | 4.68E−02 | 6.21E−02 | 2.62E−01 |
| RICTOR | NM_152756 | 2.03E−01 | 2.11E−01 | 9.07E−02 | 4.93E−02 |
| MDH1 | NM_001199112 | 2.89E−02 | 2.24E−02 | 4.47E−01 | 5.64E−02 |
| ZNF763 | NM_001012753 | 1.54E−01 | 1.66E−01 | 3.22E−02 | 2.03E−01 |
| PTGR2 | NM_152444 | 1.20E−01 | 1.31E−01 | 9.56E−02 | 2.09E−01 |
| MINPP1 | NM_001178118 | 2.61E−01 | 2.81E−01 | 7.58E−03 | 6.18E−03 |
| AQP8 | NM_001169 | 2.06E−01 | 3.48E−01 | 6.86E−04 | 1.80E−03 |
| STX3 | NM_004177 | 1.38E−01 | 4.66E−02 | 2.21E−01 | 1.50E−01 |
| MCAM | NM_006500 | 3.28E−02 | 1.17E−01 | 3.62E−01 | 4.41E−02 |
| PSG6 | NM_001031850 | 2.41E−01 | 1.36E−01 | 8.61E−02 | 9.40E−02 |
| SLC35G4 | NM_001282300 | 8.00E−02 | 8.48E−02 | 2.04E−01 | 1.89E−01 |
| FRMPD3 | NM_032428 | 1.06E−02 | 1.05E−02 | 2.23E−01 | 3.12E−01 |
| SELENOF | NM_004261 | 1.78E−01 | 1.45E−01 | 4.84E−02 | 1.85E−01 |
| CTNNA2 | NM_001282599 | 1.30E−01 | 1.38E−01 | 2.35E−01 | 5.43E−02 |
| COL19A1 | NM_001858 | 2.21E−01 | 1.14E−01 | 1.62E−01 | 6.14E−02 |
| OPRK1 | NM_000912 | 8.70E−02 | 1.90E−01 | 1.92E−01 | 8.89E−02 |
| SLC2A6 | NM_001145099 | 2.03E−01 | 4.87E−02 | 4.76E−02 | 2.60E−01 |
| GFRA2 | NM_001165038 | 2.00E−01 | 2.90E−01 | 4.04E−02 | 2.93E−02 |
| UNKL | NM_001276414 | 1.98E−01 | 1.72E−01 | 1.22E−02 | 1.78E−01 |
| JADE3 | NM_014735 | 1.56E−01 | 1.46E−01 | 9.25E−02 | 1.65E−01 |
| ZAR1 | NM_175619 | 2.02E−01 | 3.83E−02 | 1.81E−01 | 1.39E−01 |
| STAG3 | NM_012447 | 1.64E−01 | 1.41E−01 | 6.67E−02 | 1.88E−01 |
| SNX27 | NM_030918 | 2.55E−01 | 9.55E−02 | 7.40E−03 | 2.03E−01 |
| SERPINB12 | NM_080474 | 1.40E−01 | 2.79E−01 | 6.47E−02 | 7.65E−02 |
| NXPE3 | NM_001134456 | 3.55E−01 | 1.05E−01 | 9.96E−03 | 9.11E−02 |
| SEMA4B | NM_020210 | 1.01E−01 | 4.39E−01 | 1.51E−02 | 6.46E−03 |
| LHFPL1 | NM_178175 | 2.15E−01 | 1.13E−01 | 1.80E−01 | 5.40E−02 |
| ZNF92 | NM_152626 | 2.60E−01 | 2.36E−01 | 5.01E−02 | 1.57E−02 |
| CNR2 | NM_001841 | 2.02E−01 | 4.62E−02 | 4.08E−02 | 2.73E−01 |
| PRDM8 | NM_020226 | 2.95E−01 | 1.33E−01 | 2.00E−02 | 1.15E−01 |
| KLB | NM_175737 | 1.87E−01 | 2.00E−01 | 4.19E−02 | 1.34E−01 |
| FAM115A | NM_001206938 | 8.83E−02 | 6.13E−02 | 2.36E−01 | 1.77E−01 |
| TMEM95 | NM_198154 | 3.53E−01 | 1.90E−01 | 8.22E−03 | 1.18E−02 |
| SPG21 | NM_001127889 | 1.33E−01 | 1.72E−01 | 6.90E−02 | 1.90E−01 |
| SLC35E2 | NM_001199787 | 1.92E−01 | 1.75E−01 | 5.25E−03 | 1.91E−01 |
| ETFB | NM_001985 | 1.91E−01 | 3.52E−01 | 1.01E−02 | 1.11E−02 |
| HPR | NM_020995 | 4.31E−03 | 4.36E−02 | 3.30E−01 | 1.87E−01 |
| OR2T1 | NM_030904 | 1.40E−02 | 5.06E−02 | 2.44E−01 | 2.56E−01 |
| ARPP21 | NM_001267617 | 3.59E−01 | 1.50E−01 | 6.58E−03 | 4.83E−02 |
| OR9Q1 | NM_001005212 | 2.22E−01 | 2.19E−01 | 1.02E−01 | 2.21E−02 |
| ATXN1L | NM_001137675 | 2.66E−01 | 9.37E−02 | 7.65E−02 | 1.29E−01 |
| AP3B2 | NM_001278511 | 2.59E−02 | 4.75E−03 | 2.83E−01 | 2.52E−01 |
| IVL | NM_005547 | 1.13E−01 | 1.04E−01 | 3.03E−01 | 4.73E−02 |
| SCARF1 | NM_145350 | 1.15E−01 | 8.88E−02 | 1.46E−01 | 2.18E−01 |
| GPX1 | NM_000581 | 1.11E−01 | 2.68E−03 | 1.53E−01 | 3.00E−01 |
| NABP2 | NM_024068 | 1.72E−02 | 5.31E−02 | 3.30E−01 | 1.67E−01 |
| BCKDHB | NM_000056 | 2.09E−01 | 1.11E−01 | 8.21E−02 | 1.65E−01 |
| MRPL30 | NM_145212 | 1.02E−01 | 1.46E−01 | 2.06E−01 | 1.14E−01 |
| FIBP | NM_004214 | 4.81E−02 | 3.55E−02 | 4.01E−01 | 8.42E−02 |
| BCAS1 | NM_003657 | 4.71E−02 | 6.33E−02 | 3.81E−01 | 7.77E−02 |
| BMP2K | NM_198892 | 1.27E−01 | 6.69E−02 | 1.41E−01 | 2.34E−01 |
| SLC25A36 | NM_018155 | 5.59E−02 | 2.27E−01 | 1.63E−01 | 1.23E−01 |
| MACROD2 | NM_080676 | 2.84E−01 | 2.71E−01 | 5.68E−03 | 8.70E−03 |
| KDELR1 | NM_006801 | 1.93E−01 | 3.20E−01 | 5.21E−02 | 5.09E−03 |
| C3orf18 | NM_001171741 | 9.85E−02 | 1.11E−01 | 2.65E−02 | 3.34E−01 |
| CDK2AP1 | NM_001270434 | 1.08E−02 | 1.65E−02 | 3.10E−01 | 2.33E−01 |
| FCGRT | NM_001136019 | 1.97E−01 | 9.06E−02 | 2.65E−01 | 1.93E−02 |
| TNIP1 | NM_001258454 | 8.72E−02 | 6.11E−02 | 1.44E−01 | 2.80E−01 |
| SRM | NM_003132 | 1.53E−02 | 7.28E−02 | 2.82E−01 | 2.03E−01 |
| COMMD10 | NM_016144 | 2.02E−02 | 2.40E−02 | 2.88E−01 | 2.40E−01 |
| ARL4A | NM_001195396 | 1.52E−01 | 1.54E−01 | 2.27E−01 | 4.09E−02 |
| CPSF2 | NM_017437 | 9.47E−02 | 1.51E−01 | 2.04E−01 | 1.23E−01 |
| KCNN2 | NM_170775 | 2.82E−01 | 1.15E−01 | 1.07E−01 | 6.86E−02 |
| HEY2 | NM_012259 | 2.22E−01 | 1.38E−01 | 6.49E−02 | 1.49E−01 |
| PIP5K1C | NM_012398 | 1.76E−01 | 7.82E−02 | 1.41E−01 | 1.78E−01 |
| PASK | NM_001252122 | 1.28E−01 | 1.30E−01 | 5.12E−02 | 2.64E−01 |
| TBC1D10C | NM_198517 | 9.11E−02 | 1.36E−01 | 3.10E−01 | 3.66E−02 |
| DGCR6 | NM_005675 | 2.40E−01 | 1.19E−01 | 1.51E−01 | 6.43E−02 |
| CHST6 | NM_021615 | 2.52E−01 | 2.86E−01 | 1.86E−02 | 1.91E−02 |
| SCML4 | NM_001286409 | 8.26E−02 | 1.14E−01 | 2.53E−01 | 1.26E−01 |
| HOOK2 | NM_001100176 | 2.98E−01 | 2.52E−01 | 9.64E−03 | 1.66E−02 |
| MLC1 | NM_015166 | 1.96E−01 | 2.33E−01 | 9.23E−02 | 5.52E−02 |
| SMC5 | NM_015110 | 1.94E−01 | 1.36E−01 | 1.05E−01 | 1.42E−01 |
| MARCHF5 | NM_017824 | 2.68E−01 | 1.36E−01 | 1.59E−01 | 1.45E−02 |
| WNT5B | NM_030775 | 1.29E−01 | 3.56E−01 | 2.84E−02 | 6.40E−02 |
| CSNK1G2 | NM_001319 | 9.37E−02 | 1.03E−01 | 1.57E−01 | 2.24E−01 |
| C6orf223 | NM_153246 | 4.82E−02 | 9.72E−02 | 8.35E−02 | 3.49E−01 |
| HNRNPL | NM_001533 | 1.42E−01 | 1.60E−01 | 1.48E−01 | 1.29E−01 |
| CMTM4 | NM_181521 | 2.27E−01 | 6.97E−02 | 1.36E−01 | 1.46E−01 |
| NOTCH2 | NM_024408 | 2.03E−01 | 2.87E−01 | 4.97E−02 | 4.02E−02 |
| EPPIN-WFDC6 | NM_001198986 | 1.01E−02 | 5.12E−02 | 2.34E−01 | 2.85E−01 |
| NOP16 | NM_001256539 | 2.82E−01 | 2.60E−01 | 2.76E−02 | 1.16E−02 |
| SPAM1 | NM_001174045 | 7.89E−02 | 3.57E−01 | 5.63E−02 | 8.93E−02 |
| ATP6V1H | NM_213620 | 1.18E−01 | 8.14E−02 | 2.10E−01 | 1.72E−01 |
| NFS1 | NM_001198989 | 1.80E−01 | 7.23E−02 | 8.66E−02 | 2.43E−01 |
| CBFA2T3 | NM_005187 | 1.08E−02 | 1.37E−02 | 1.62E−01 | 3.96E−01 |
| HS3ST4 | NM_006040 | 5.64E−02 | 6.40E−02 | 1.31E−01 | 3.31E−01 |
| C18orf21 | NM_001201476 | 2.25E−02 | 8.46E−03 | 4.52E−01 | 9.95E−02 |
| APOLD1 | NM_030817 | 1.25E−01 | 1.07E−01 | 2.87E−01 | 6.31E−02 |
| KLHL22 | NM_032775 | 1.69E−01 | 7.74E−02 | 1.48E−01 | 1.88E−01 |
| CCDC136 | NM_001201372 | 5.97E−02 | 4.65E−02 | 1.89E−01 | 2.88E−01 |
| KIAA1024 | NM_015206 | 2.83E−01 | 1.26E−01 | 7.02E−02 | 1.03E−01 |
| NKG7 | NM_005601 | 3.78E−02 | 3.03E−02 | 3.88E−01 | 1.27E−01 |
| TTC7A | NM_020458 | 2.58E−01 | 1.53E−01 | 2.59E−02 | 1.47E−01 |
| ATP1A3 | NM_001256214 | 2.08E−01 | 2.42E−01 | 3.13E−02 | 1.03E−01 |
| BATF2 | NM_138456 | 2.07E−01 | 3.04E−01 | 6.63E−02 | 6.99E−03 |
| SPANXA1 | NM_013453 | 1.63E−01 | 1.16E−01 | 8.14E−02 | 2.24E−01 |
| ID1 | NM_181353 | 1.32E−01 | 3.91E−01 | 2.57E−02 | 3.55E−02 |
| QTRTD1 | NM_024638 | 2.00E−02 | 1.31E−01 | 2.94E−01 | 1.39E−01 |
| AIMP1 | NM_001142415 | 2.25E−01 | 2.18E−01 | 7.84E−02 | 6.35E−02 |
| RASGRF2 | NM_006909 | 1.21E−01 | 1.56E−01 | 5.19E−02 | 2.56E−01 |
| KHDRBS1 | NM_006559 | 2.88E−01 | 1.30E−01 | 7.89E−02 | 8.78E−02 |
| LCMT2 | NM_014793 | 1.81E−01 | 2.82E−01 | 5.22E−02 | 6.98E−02 |
| EXOC6 | NM_019053 | 8.91E−02 | 1.87E−01 | 1.75E−01 | 1.34E−01 |
| NIF3L1 | NM_021824 | 3.36E−02 | 9.66E−03 | 3.23E−01 | 2.19E−01 |
| PSMB1 | NM_002793 | 6.99E−02 | 1.30E−01 | 5.97E−02 | 3.26E−01 |
| DOC2A | NM_003586 | 1.46E−01 | 4.81E−02 | 2.35E−01 | 1.56E−01 |
| NUDT10 | NM_153183 | 1.93E−01 | 2.48E−01 | 7.28E−02 | 7.19E−02 |
| RAC3 | NM_005052 | 7.91E−02 | 1.11E−02 | 1.39E−01 | 3.57E−01 |
| SDHA | NM_004168 | 4.13E−02 | 2.82E−02 | 2.35E−01 | 2.83E−01 |
| GPBAR1 | NM_170699 | 2.71E−01 | 4.64E−02 | 1.55E−01 | 1.15E−01 |
| AIFM3 | NM_001146288 | 1.76E−01 | 3.09E−01 | 8.29E−02 | 1.90E−02 |
| CCDC94 | NM_018074 | 1.04E−01 | 1.36E−01 | 1.61E−01 | 1.86E−01 |
| NFKBIE | NM_004556 | 3.59E−02 | 5.07E−01 | 8.56E−03 | 3.69E−02 |
| HDLBP | NM_203346 | 1.32E−01 | 2.03E−01 | 2.26E−01 | 2.65E−02 |
| KCNQ3 | NM_004519 | 7.52E−02 | 1.96E−02 | 2.31E−01 | 2.63E−01 |
| SLURP1 | NM_020427 | 1.80E−01 | 6.85E−02 | 1.74E−01 | 1.66E−01 |
| PTPRB | NM_001206971 | 1.42E−01 | 2.21E−01 | 4.33E−02 | 1.83E−01 |
| NR1H4 | NM_001206993 | 6.83E−02 | 5.66E−03 | 1.83E−01 | 3.32E−01 |
| CXorf23 | NM_198279 | 1.91E−01 | 2.81E−01 | 9.98E−02 | 1.79E−02 |
| HOXB13 | NM_006361 | 2.52E−02 | 1.59E−02 | 3.16E−01 | 2.33E−01 |
| FCAR | NM_133278 | 3.50E−01 | 1.19E−01 | 2.21E−02 | 9.90E−02 |
| GPT | NM_005309 | 3.63E−03 | 1.99E−02 | 2.88E−01 | 2.80E−01 |
| GLIPR1 | NM_006851 | 9.36E−02 | 1.29E−01 | 2.47E−01 | 1.22E−01 |
| PP2D1 | NM_001252657 | 7.97E−02 | 7.64E−02 | 2.14E−01 | 2.21E−01 |
| FUT6 | NM_000150 | 2.69E−01 | 3.20E−01 | 1.51E−03 | 8.09E−04 |
| IFT140 | NM_014714 | 3.23E−02 | 3.77E−01 | 1.24E−01 | 5.79E−02 |
| SIN3B | NM_015260 | 8.64E−02 | 1.73E−01 | 1.71E−01 | 1.61E−01 |
| UXS1 | NM_001253875 | 1.63E−01 | 4.04E−01 | 1.90E−02 | 6.29E−03 |
| SLC6A12 | NM_001206931 | 1.38E−01 | 1.07E−01 | 2.15E−01 | 1.32E−01 |
| SYT11 | NM_152280 | 1.24E−01 | 1.22E−01 | 1.81E−01 | 1.67E−01 |
| MRPS10 | NM_018141 | 2.42E−02 | 6.26E−02 | 2.25E−01 | 2.82E−01 |
| TMC2 | NM_080751 | 1.72E−01 | 3.25E−01 | 2.59E−02 | 7.20E−02 |
| IP6K3 | NM_054111 | 1.02E−01 | 2.76E−01 | 1.28E−01 | 8.93E−02 |
| NCAPD2 | NM_014865 | 8.87E−02 | 1.56E−01 | 2.42E−01 | 1.09E−01 |
| C15orf41 | NM_001130010 | 8.93E−02 | 8.09E−02 | 2.20E−01 | 2.04E−01 |
| ZNF200 | NM_003454 | 6.11E−02 | 6.08E−02 | 2.69E−01 | 2.04E−01 |
| ACADL | NM_001608 | 1.72E−01 | 6.28E−02 | 2.14E−01 | 1.47E−01 |
| GP1BB | NM_000407 | 3.17E−01 | 1.64E−01 | 1.10E−01 | 5.79E−03 |
| TTLL11 | NM_001139442 | 4.77E−02 | 2.58E−02 | 3.52E−01 | 1.71E−01 |
| PRR5 | NM_001017528 | 1.49E−01 | 3.72E−01 | 2.55E−02 | 5.02E−02 |
| BMPR2 | NM_001204 | 8.93E−02 | 3.74E−01 | 8.66E−03 | 1.24E−01 |
| PTGES3 | NM_006601 | 1.78E−01 | 1.37E−01 | 1.22E−01 | 1.60E−01 |
| BDKRB1 | NM_000710 | 8.75E−02 | 5.02E−02 | 2.07E−01 | 2.54E−01 |
| NXF1 | NM_001081491 | 1.13E−01 | 7.40E−02 | 1.14E−01 | 2.97E−01 |
| HAND2 | NM_021973 | 1.96E−01 | 1.82E−01 | 4.20E−02 | 1.78E−01 |
| GRID2 | NM_001286838 | 3.77E−01 | 6.98E−02 | 8.10E−02 | 7.16E−02 |
| KLHL30 | NM_198582 | 9.96E−02 | 1.62E−01 | 2.29E−01 | 1.09E−01 |
| CCR6 | NM_004367 | 3.74E−02 | 7.64E−02 | 1.08E−01 | 3.77E−01 |
| KIAA0368 | NM_001080398 | 1.20E−01 | 1.68E−01 | 7.28E−02 | 2.39E−01 |
| PRPF38A | NM_032864 | 1.05E−01 | 1.10E−01 | 1.38E−01 | 2.46E−01 |
| MAP3K13 | NM_004721 | 2.60E−01 | 3.02E−01 | 9.79E−03 | 2.79E−02 |
| CORO6 | NM_032854 | 3.60E−01 | 1.92E−01 | 2.78E−02 | 2.05E−02 |
| ARHGEF25 | NM_001111270 | 2.64E−01 | 1.63E−01 | 7.49E−02 | 9.78E−02 |
| AMOTL2 | NM_001278683 | 2.56E−01 | 2.33E−01 | 7.09E−02 | 4.02E−02 |
| TRMT5 | NM_020810 | 3.31E−02 | 1.59E−01 | 5.95E−02 | 3.50E−01 |
| ARSF | NM_001201538 | 4.17E−01 | 8.71E−02 | 5.06E−02 | 4.65E−02 |
| PACS2 | NM_001100913 | 1.64E−01 | 9.48E−02 | 1.86E−01 | 1.56E−01 |
| LSM7 | NM_016199 | 8.05E−02 | 1.88E−01 | 1.59E−01 | 1.74E−01 |
| MMP11 | NM_005940 | 2.87E−02 | 1.95E−02 | 4.01E−01 | 1.52E−01 |
| SLC25A37 | NM_016612 | 3.33E−02 | 2.85E−01 | 1.41E−01 | 1.43E−01 |
| KRT14 | NM_000526 | 1.31E−03 | 6.86E−04 | 2.96E−01 | 3.04E−01 |
| ANKRD37 | NM_181726 | 6.98E−02 | 3.64E−02 | 2.50E−01 | 2.47E−01 |
| EPHA7 | NM_001288629 | 3.13E−02 | 9.11E−02 | 2.54E−01 | 2.28E−01 |
| CSTF1 | NM_001033521 | 8.49E−03 | 4.90E−03 | 2.01E−01 | 3.90E−01 |
| SPATA13 | NM_153023 | 3.45E−02 | 1.05E−01 | 1.65E−01 | 3.01E−01 |
| HN1 | NM_016185 | 2.11E−01 | 1.44E−01 | 2.05E−01 | 4.55E−02 |
| PARD6G | NM_032510 | 3.63E−01 | 1.57E−01 | 5.02E−02 | 3.56E−02 |
| GAPDHS | NM_014364 | 1.67E−01 | 3.86E−01 | 3.87E−02 | 1.32E−02 |
| SYNCRIP | NM_001159673 | 2.39E−01 | 4.78E−02 | 6.42E−02 | 2.55E−01 |
| MXI1 | NM_130439 | 3.71E−01 | 2.25E−01 | 2.06E−03 | 7.74E−03 |
| DMPK | NM_001081563 | 2.70E−01 | 6.36E−02 | 1.52E−01 | 1.20E−01 |
| IPO5 | NM_002271 | 3.00E−01 | 9.77E−02 | 8.80E−02 | 1.21E−01 |
| ATP10B | NM_025153 | 4.20E−01 | 1.35E−01 | 7.80E−03 | 4.36E−02 |
| RPL17-C18orf32 | NM_001199355 | 2.48E−01 | 1.66E−01 | 7.95E−02 | 1.13E−01 |
| TRIM42 | NM_152616 | 1.60E−01 | 1.51E−01 | 2.75E−02 | 2.69E−01 |
| CDC20 | NM_001255 | 3.47E−01 | 2.53E−01 | 2.75E−03 | 4.93E−03 |
| ATF3 | NM_001030287 | 1.39E−01 | 2.04E−01 | 1.80E−01 | 8.59E−02 |
| FARS2 | NM_006567 | 1.93E−01 | 1.84E−01 | 2.05E−01 | 2.65E−02 |
| MYO1C | NM_033375 | 6.64E−03 | 1.74E−02 | 2.74E−01 | 3.10E−01 |
| POLR1D | NM_001206559 | 2.42E−01 | 2.22E−01 | 9.06E−02 | 5.47E−02 |
| LUC7L2 | NM_001270643 | 5.69E−02 | 9.42E−02 | 2.52E−01 | 2.07E−01 |
| RASAL1 | NM_001193520 | 1.59E−01 | 1.49E−01 | 1.69E−01 | 1.33E−01 |
| DNAJC25-GNG10 | NM_004125 | 9.52E−02 | 2.01E−01 | 1.91E−01 | 1.23E−01 |
| MAGEB1 | NM_002363 | 1.51E−01 | 2.86E−01 | 1.65E−01 | 8.56E−03 |
| TJP2 | NM_001170414 | 2.68E−01 | 1.78E−01 | 4.08E−02 | 1.24E−01 |
| ATP1A1 | NM_001160233 | 4.31E−02 | 6.71E−02 | 4.31E−01 | 6.93E−02 |
| RRM2 | NM_001165931 | 9.44E−02 | 1.94E−02 | 2.97E−01 | 2.00E−01 |
| CCDC50 | NM_178335 | 1.77E−02 | 5.74E−03 | 3.82E−01 | 2.06E−01 |
| CTDSP1 | NM_001206878 | 6.09E−02 | 1.89E−01 | 1.06E−01 | 2.56E−01 |
| TMEM89 | NM_001008269 | 6.32E−02 | 1.46E−01 | 1.53E−01 | 2.50E−01 |
| BICD2 | NM_015250 | 1.57E−01 | 2.77E−01 | 1.57E−01 | 2.20E−02 |
| KLHL25 | NM_022480 | 2.31E−01 | 3.78E−02 | 2.73E−01 | 7.20E−02 |
| CCDC33 | NM_025055 | 1.51E−01 | 2.59E−01 | 1.06E−01 | 9.79E−02 |
| SNX32 | NM_152760 | 3.54E−01 | 1.39E−01 | 2.66E−02 | 9.35E−02 |
| MATR3 | NM_018834 | 2.70E−01 | 8.08E−02 | 3.01E−02 | 2.34E−01 |
| SLC46A3 | NM_001135919 | 2.38E−01 | 1.43E−01 | 9.04E−02 | 1.43E−01 |
| NLRC4 | NM_001199139 | 1.69E−01 | 8.32E−02 | 3.03E−01 | 5.84E−02 |
| PSMF1 | NM_178578 | 4.61E−02 | 1.38E−01 | 3.41E−04 | 4.30E−01 |
| CPNE9 | NM_153635 | 2.83E−02 | 9.97E−03 | 2.88E−01 | 2.89E−01 |
| KLF11 | NM_001177716 | 2.12E−01 | 8.38E−02 | 1.35E−01 | 1.85E−01 |
| CSN3 | NM_005212 | 1.39E−01 | 1.09E−01 | 1.87E−01 | 1.82E−01 |
| TRIM39 | NM_021253 | 2.79E−01 | 3.26E−01 | 7.46E−03 | 4.01E−03 |
| YY1AP1 | NM_001198903 | 1.37E−01 | 1.13E−01 | 3.06E−01 | 6.13E−02 |
| BTN2A1 | NM_001197234 | 3.01E−02 | 5.61E−02 | 2.67E−01 | 2.64E−01 |
| SMARCAD1 | NM_001254949 | 1.80E−03 | 3.11E−03 | 1.15E−01 | 4.98E−01 |
| PHYHIPL | NM_001143774 | 1.77E−02 | 2.24E−02 | 1.32E−01 | 4.45E−01 |
| PHACTR4 | NM_023923 | 1.07E−02 | 1.71E−03 | 2.45E−01 | 3.62E−01 |
| LTA4H | NM_000895 | 5.58E−02 | 5.70E−02 | 1.02E−01 | 4.05E−01 |
| TMEM30B | NM_001017970 | 1.72E−01 | 1.99E−01 | 1.26E−01 | 1.22E−01 |
| CCDC9 | NM_015603 | 1.39E−01 | 1.01E−01 | 1.74E−01 | 2.05E−01 |
| NOV | NM_002514 | 3.30E−01 | 1.38E−01 | 8.11E−02 | 7.08E−02 |
| NDUFB5 | NM_002492 | 2.83E−01 | 2.98E−02 | 1.27E−01 | 1.80E−01 |
| ARHGEF33 | NM_001145451 | 2.43E−01 | 2.49E−01 | 4.59E−02 | 8.27E−02 |
| ALG1 | NM_019109 | 1.40E−01 | 2.63E−01 | 1.73E−01 | 4.46E−02 |
| MAP4K4 | NM_001242559 | 2.72E−01 | 8.06E−02 | 3.90E−02 | 2.28E−01 |
| KCTD8 | NM_198353 | 2.39E−01 | 1.20E−01 | 2.08E−01 | 5.38E−02 |
| PDE4D | NM_001197219 | 2.14E−01 | 1.52E−01 | 1.27E−01 | 1.29E−01 |
| PCDHGA4 | NM_018917 | 7.76E−02 | 5.51E−02 | 4.20E−01 | 6.89E−02 |
| OPN1SW | NM_001708 | 7.01E−02 | 1.83E−02 | 3.88E−01 | 1.45E−01 |
| YBEY | NM_058181 | 8.97E−02 | 1.22E−01 | 2.15E−01 | 1.95E−01 |
| ASRGL1 | NM_001083926 | 7.12E−02 | 2.39E−02 | 2.65E−01 | 2.62E−01 |
| NDUFAB1 | NM_005003 | 3.69E−01 | 2.24E−01 | 1.71E−02 | 1.22E−02 |
| GGTLC1 | NM_178312 | 1.20E−01 | 2.53E−01 | 1.75E−01 | 7.48E−02 |
| FIZ1 | NM_032836 | 2.69E−01 | 3.23E−01 | 6.53E−03 | 2.55E−02 |
| LMNA | NM_001282625 | 9.67E−02 | 5.60E−02 | 1.12E−01 | 3.60E−01 |
| NUBP1 | NM_002484 | 2.10E−01 | 2.63E−01 | 1.38E−01 | 1.27E−02 |
| ATF2 | NM_001256093 | 3.71E−01 | 1.97E−01 | 2.08E−02 | 3.55E−02 |
| GNRH2 | NM_001501 | 1.37E−01 | 3.57E−01 | 1.89E−02 | 1.13E−01 |
| TPM3 | NM_153649 | 1.48E−01 | 2.84E−01 | 1.23E−01 | 7.10E−02 |
| NRROS | NM_198565 | 3.68E−02 | 8.08E−02 | 1.66E−01 | 3.42E−01 |
| OVGP1 | NM_002557 | 6.05E−02 | 3.66E−02 | 2.17E−01 | 3.11E−01 |
| ATP2A1 | NM_173201 | 2.44E−01 | 2.28E−01 | 4.08E−02 | 1.13E−01 |
| SERF2 | NM_001199878 | 4.81E−04 | 1.95E−04 | 4.01E−01 | 2.25E−01 |
| TRIB1 | NM_001282985 | 2.62E−01 | 2.47E−01 | 3.59E−02 | 8.09E−02 |
| CCDC61 | NM_001267723 | 3.35E−03 | 1.36E−02 | 2.06E−01 | 4.04E−01 |
| WDR1 | NM_017491 | 1.42E−01 | 3.80E−01 | 5.61E−03 | 9.90E−02 |
| FBXO9 | NM_033481 | 2.29E−01 | 3.40E−01 | 1.05E−02 | 4.71E−02 |
| VWA5A | NM_014622 | 9.29E−02 | 4.16E−02 | 3.30E−01 | 1.63E−01 |
| C1orf146 | NM_001012425 | 3.37E−01 | 2.82E−01 | 8.78E−05 | 8.25E−03 |
| ZAN | NM_003386 | 3.39E−01 | 2.04E−01 | 3.23E−02 | 5.40E−02 |
| PLA2G4C | NM_001159323 | 4.19E−01 | 1.17E−01 | 1.45E−02 | 7.86E−02 |
| SHFM1 | NM_006304 | 9.60E−02 | 1.51E−01 | 2.02E−01 | 1.81E−01 |
| ANAPC11 | NM_001002245 | 2.86E−02 | 8.95E−03 | 2.06E−01 | 3.86E−01 |
| APOA1BP | NM_144772 | 1.11E−01 | 2.44E−01 | 1.60E−01 | 1.15E−01 |
| SUPT4H1 | NM_003168 | 2.24E−01 | 2.68E−01 | 1.07E−01 | 3.11E−02 |
| KLHL13 | NM_001168299 | 4.34E−02 | 1.60E−01 | 3.36E−01 | 9.02E−02 |
| DCX | NM_000555 | 1.83E−01 | 1.88E−01 | 1.14E−01 | 1.45E−01 |
| CLDN10 | NM_001160100 | 3.74E−01 | 2.16E−01 | 8.37E−03 | 3.14E−02 |
| SMIM2 | NM_024058 | 6.56E−02 | 4.02E−01 | 9.11E−02 | 7.11E−02 |
| EBF2 | NM_022659 | 1.73E−02 | 1.06E−01 | 2.30E−01 | 2.77E−01 |
| TRPM3 | NM_001007471 | 1.76E−01 | 1.71E−01 | 8.24E−02 | 2.00E−01 |
| HBZ | NM_005332 | 3.11E−03 | 2.87E−03 | 3.48E−01 | 2.77E−01 |
| TRADD | NM_003789 | 7.20E−02 | 7.47E−02 | 2.96E−02 | 4.55E−01 |
| GNAI2 | NM_002070 | 1.14E−01 | 2.08E−01 | 1.82E−01 | 1.28E−01 |
| KIF12 | NM_138424 | 2.30E−01 | 2.04E−01 | 1.21E−01 | 7.68E−02 |
| OR5AC2 | NM_054106 | 1.23E−01 | 1.19E−01 | 2.19E−01 | 1.71E−01 |
| SCTR | NM_002980 | 1.19E−01 | 9.75E−02 | 1.55E−01 | 2.60E−01 |
| LRRC27 | NM_001143757 | 1.08E−01 | 3.65E−02 | 2.98E−01 | 1.90E−01 |
| ZNF410 | NM_001242924 | 1.73E−01 | 4.07E−01 | 3.29E−02 | 2.00E−02 |
| CLECL1 | NM_001253750 | 1.27E−01 | 4.62E−02 | 3.86E−01 | 7.36E−02 |
| NPHS2 | NM_014625 | 9.01E−02 | 1.08E−01 | 3.02E−01 | 1.33E−01 |
| C16orf13 | NM_032366 | 2.34E−01 | 2.32E−01 | 6.54E−02 | 1.03E−01 |
| CDKN1A | NM_078467 | 7.18E−02 | 4.84E−02 | 1.60E−01 | 3.54E−01 |
| APOF | NM_001638 | 1.34E−01 | 4.23E−01 | 2.77E−02 | 4.99E−02 |
| BSCL2 | NM_032667 | 3.29E−01 | 9.02E−02 | 1.00E−01 | 1.16E−01 |
| BIRC5 | NM_001168 | 1.37E−01 | 1.27E−02 | 2.11E−01 | 2.74E−01 |
| PRKACB | NM_182948 | 8.76E−02 | 1.49E−01 | 2.13E−01 | 1.86E−01 |
| ALAS2 | NM_001037968 | 9.40E−02 | 4.09E−02 | 3.39E−01 | 1.61E−01 |
| COL10A1 | NM_000493 | 3.45E−01 | 2.82E−01 | 8.68E−03 | 1.36E−04 |
| SLC2A5 | NM_003039 | 4.39E−02 | 3.04E−02 | 2.04E−01 | 3.57E−01 |
| ELAVL4 | NM_001144775 | 1.93E−01 | 3.22E−01 | 1.43E−02 | 1.07E−01 |
| LIMS1 | NM_004987 | 3.17E−01 | 3.00E−01 | 1.17E−02 | 7.80E−03 |
| PKHD1 | NM_138694 | 2.99E−02 | 5.26E−03 | 1.78E−01 | 4.23E−01 |
| NUP188 | NM_015354 | 1.10E−01 | 1.36E−02 | 1.99E−01 | 3.14E−01 |
| OR4C15 | NM_001001920 | 2.04E−01 | 9.00E−02 | 1.10E−01 | 2.33E−01 |
| PABPC4L | NM_001114734 | 6.25E−02 | 4.84E−02 | 1.33E−01 | 3.93E−01 |
| TM7SF2 | NM_001277233 | 2.67E−01 | 2.55E−01 | 7.00E−02 | 4.53E−02 |
| POC5 | NM_001099271 | 1.62E−01 | 1.29E−01 | 2.76E−01 | 7.11E−02 |
| C1orf86 | NM_182533 | 3.00E−01 | 2.63E−01 | 6.42E−02 | 1.22E−02 |
| SBSN | NM_001166034 | 2.93E−01 | 3.02E−01 | 2.91E−03 | 4.14E−02 |
| DEF8 | NM_001242821 | 3.38E−01 | 2.16E−01 | 2.35E−02 | 6.21E−02 |
| APLP1 | NM_005166 | 1.02E−01 | 5.84E−02 | 3.34E−01 | 1.45E−01 |
| DCK | NM_000788 | 1.60E−01 | 4.12E−01 | 5.54E−03 | 6.24E−02 |
| CLDN24 | NM_001185149 | 2.46E−01 | 1.37E−01 | 1.37E−01 | 1.20E−01 |
| CETN2 | NM_004344 | 2.12E−01 | 2.36E−01 | 1.29E−01 | 6.43E−02 |
| CTSA | NM_001127695 | 3.13E−02 | 5.49E−03 | 2.78E−01 | 3.27E−01 |

TABLE 1B
MAGeCK analysis results of the CRISPRa screen for resistance to T cell cytotoxicity
in the chronic screen. Gene targets are in ranked order. Gene names, RefSeq IDs,and
MAGeCK P-values for each of the top 1000 screening bioreps are listed.

| | | Chronic | Chronic | Chronic | Chronic | Chronic | Chronic |
|---|---|---|---|---|---|---|---|
| | | Round 1 | Round 1 | Round 2 | Round 2 | Round3 | Round 3 |
| Gene | id | biorep 1 | biorep 2 | biorep 1 | biorep 2 | biorep 1 | biorep 2 |
| JUNB | NM_002229 | 2.28E−03 | 4.31E−03 | 2.05E−03 | 1.97E−04 | 2.25E−03 | 3.57E−03 |
| B3GNT2 | NM_006577 | 1.24E−02 | 2.28E−04 | 7.91E−03 | 5.20E−05 | 2.64E−03 | 1.29E−04 |
| INO80 | NM_017553 | 3.59E−03 | 9.27E−03 | 3.23E−03 | 5.03E−04 | 1.06E−02 | 1.55E−03 |
| KRBA1 | NM_001290187 | 4.50E−04 | 4.23E−02 | 1.61E−03 | 4.58E−03 | 3.97E−03 | 2.85E−03 |
| ADAMTS12 | NM_030955 | 3.65E−02 | 8.21E−04 | 6.75E−03 | 7.50E−03 | 2.00E−03 | 1.14E−02 |
| RBPMS2 | NM_194272 | 3.53E−02 | 4.52E−02 | 3.57E−03 | 6.45E−03 | 2.23E−03 | 2.15E−02 |
| WDR76 | NM_001167941 | 1.45E−02 | 9.20E−02 | 2.01E−04 | 3.06E−03 | 1.55E−03 | 3.71E−02 |
| STK40 | NM_032017 | 8.27E−02 | 5.61E−03 | 1.03E−02 | 2.58E−02 | 1.41E−02 | 9.75E−03 |
| GABBR1 | NM_021904 | 2.11E−02 | 1.20E−01 | 3.46E−03 | 5.41E−03 | 1.40E−03 | 2.27E−02 |
| C2orf88 | NM_001042520 | 9.24E−03 | 2.04E−02 | 3.87E−02 | 2.40E−02 | 6.17E−02 | 2.11E−02 |
| GPR26 | NM_153442 | 7.08E−02 | 4.32E−02 | 2.89E−02 | 1.24E−02 | 1.20E−02 | 1.21E−02 |
| KCNF1 | NM_002236 | 3.67E−02 | 1.06E−01 | 3.11E−03 | 4.65E−03 | 1.52E−02 | 1.64E−02 |
| HHATL | NM_020707 | 2.40E−03 | 3.15E−03 | 3.95E−02 | 3.67E−02 | 1.80E−02 | 9.34E−02 |
| ATP2A2 | NM_170665 | 1.95E−03 | 1.41E−01 | 2.16E−02 | 5.44E−03 | 1.35E−02 | 1.09E−02 |
| EFNA1 | NM_004428 | 8.43E−02 | 3.64E−02 | 1.07E−02 | 5.77E−02 | 7.46E−03 | 1.17E−03 |
| CIDEC | NM_001199623 | 1.24E−01 | 3.89E−03 | 6.36E−02 | 1.56E−03 | 7.17E−03 | 7.74E−05 |
| DKK2 | NM_014421 | 2.60E−02 | 8.08E−02 | 3.96E−06 | 3.52E−02 | 2.30E−06 | 5.89E−02 |
| ZNF497 | NM_001207009 | 4.06E−03 | 2.00E−03 | 1.98E−02 | 8.65E−02 | 2.90E−02 | 6.50E−02 |
| TNS3 | NM_022748 | 1.62E−01 | 1.34E−03 | 1.84E−02 | 1.17E−02 | 9.65E−03 | 5.70E−03 |
| KCNH7 | NM_033272 | 5.14E−02 | 6.60E−02 | 3.03E−02 | 2.61E−02 | 1.32E−02 | 2.55E−02 |
| CLDN4 | NM_001305 | 3.25E−02 | 5.24E−02 | 6.73E−03 | 5.57E−02 | 1.68E−02 | 6.25E−02 |
| AP3M1 | NM_207012 | 1.59E−01 | 2.00E−02 | 4.78E−03 | 3.71E−02 | 2.48E−03 | 7.11E−03 |
| NME2 | NM_001018139 | 4.11E−02 | 1.73E−02 | 1.93E−04 | 1.84E−02 | 6.93E−04 | 1.55E−01 |
| RBBP8 | NM_002894 | 4.58E−02 | 2.61E−02 | 4.78E−02 | 1.12E−03 | 1.12E−01 | 1.54E−03 |
| NIPAL1 | NM_207330 | 2.04E−02 | 2.82E−02 | 4.89E−02 | 2.23E−02 | 8.06E−02 | 4.06E−02 |
| IQSEC3 | NM_015232 | 4.36E−02 | 1.76E−02 | 3.18E−02 | 9.93E−02 | 2.16E−02 | 2.73E−02 |
| FEZ2 | NM_005102 | 6.68E−02 | 1.07E−01 | 7.59E−03 | 2.21E−02 | 1.76E−02 | 2.56E−02 |
| ERMN | NM_001009959 | 1.31E−02 | 9.99E−02 | 1.16E−02 | 2.89E−02 | 1.60E−02 | 7.95E−02 |
| NYNRIN | NM_025081 | 4.67E−02 | 6.86E−02 | 1.73E−02 | 3.81E−02 | 1.58E−02 | 6.40E−02 |
| ACTG1 | NM_001199954 | 1.37E−01 | 2.70E−02 | 2.64E−02 | 2.45E−02 | 6.31E−03 | 3.26E−02 |
| NELFA | NM_005663 | 2.23E−02 | 2.89E−02 | 2.75E−02 | 1.02E−01 | 2.52E−02 | 4.85E−02 |
| ACTA1 | NM_001100 | 5.13E−02 | 1.72E−02 | 9.57E−02 | 1.71E−02 | 5.12E−02 | 2.63E−02 |
| MAGEA4 | NM_001011549 | 5.60E−03 | 1.51E−01 | 1.18E−03 | 2.96E−02 | 4.93E−03 | 6.64E−02 |
| ESRRG | NM_001134285 | 1.35E−01 | 4.08E−02 | 2.07E−02 | 2.23E−03 | 5.84E−02 | 4.79E−03 |
| ITLN1 | NM_017625 | 8.31E−02 | 2.37E−02 | 8.43E−03 | 4.69E−02 | 5.74E−03 | 9.54E−02 |
| ECHDC1 | NM_018479 | 1.85E−02 | 2.07E−03 | 5.00E−02 | 1.56E−01 | 4.40E−02 | 2.18E−03 |
| RALGDS | NM_001271774 | 1.43E−01 | 3.31E−02 | 3.50E−02 | 4.91E−03 | 5.59E−02 | 6.87E−03 |
| LGALS14 | NM_203471 | 1.73E−02 | 6.99E−03 | 1.55E−01 | 1.75E−02 | 6.06E−02 | 2.34E−02 |
| SGPP2 | NM_152386 | 1.71E−02 | 7.69E−02 | 1.92E−02 | 7.45E−02 | 3.20E−02 | 6.46E−02 |
| CCDC66 | NM_001141947 | 1.08E−01 | 1.09E−02 | 4.28E−02 | 6.26E−04 | 1.24E−01 | 1.34E−03 |
| ZNF576 | NM_024327 | 1.13E−01 | 1.19E−01 | 1.96E−02 | 2.82E−02 | 4.27E−03 | 6.06E−03 |
| MACROD2 | NM_001033087 | 2.70E−02 | 7.67E−02 | 7.80E−02 | 7.41E−02 | 1.83E−02 | 1.59E−02 |
| HMGB2 | NM_001130689 | 3.64E−02 | 1.03E−01 | 4.11E−02 | 4.10E−03 | 1.04E−01 | 2.64E−03 |
| JUN | NM_002228 | 2.84E−02 | 1.12E−02 | 3.59E−02 | 9.50E−02 | 8.28E−02 | 5.26E−02 |
| PPP2R2C | NM_001206995 | 1.56E−02 | 1.14E−02 | 1.07E−01 | 4.14E−02 | 1.13E−01 | 2.15E−02 |
| RCAN2 | NM_001251974 | 2.02E−02 | 1.20E−01 | 3.70E−03 | 4.13E−02 | 8.05E−03 | 1.22E−01 |
| SATB1 | NM_001195470 | 9.02E−03 | 9.09E−02 | 3.55E−03 | 1.35E−01 | 2.81E−02 | 4.92E−02 |
| F8A2 | NM_001007523 | 6.75E−02 | 4.82E−02 | 3.37E−02 | 6.40E−02 | 3.24E−02 | 7.14E−02 |
| MCRS1 | NM_006337 | 8.90E−02 | 4.55E−02 | 3.75E−02 | 5.73E−02 | 3.25E−02 | 5.55E−02 |
| CCDC160 | NM_001101357 | 5.70E−02 | 2.94E−02 | 1.96E−02 | 7.44E−02 | 1.61E−02 | 1.22E−01 |
| USP10 | NM_001272075 | 6.17E−02 | 1.98E−02 | 4.55E−02 | 4.41E−02 | 6.08E−02 | 8.80E−02 |
| LAD1 | NM_005558 | 6.17E−02 | 8.25E−02 | 3.17E−02 | 6.39E−02 | 2.46E−02 | 5.90E−02 |
| CCBL2 | NM_001008661 | 3.42E−03 | 1.34E−01 | 2.68E−02 | 4.00E−02 | 9.51E−02 | 2.58E−02 |
| FAM118A | NM_017911 | 1.38E−03 | 2.79E−01 | 2.12E−03 | 9.03E−03 | 2.28E−02 | 1.14E−02 |
| RINT1 | NM_021930 | 1.25E−01 | 7.92E−04 | 8.66E−02 | 5.51E−02 | 5.91E−02 | 2.85E−03 |
| SF3A2 | NM_007165 | 6.61E−02 | 1.77E−01 | 2.10E−02 | 2.95E−02 | 5.49E−03 | 3.09E−02 |
| ASXL3 | NM_030632 | 9.94E−02 | 2.35E−02 | 1.95E−03 | 7.64E−02 | 2.54E−03 | 1.28E−01 |
| PCDH7 | NM_032457 | 9.21E−02 | 2.31E−02 | 3.66E−02 | 8.02E−02 | 1.59E−02 | 8.37E−02 |
| HNRNPF | NM_001098204 | 1.70E−02 | 4.06E−02 | 1.39E−01 | 4.74E−02 | 8.09E−02 | 1.35E−02 |
| ZNF728 | NM_001267716 | 7.66E−02 | 5.18E−02 | 7.22E−03 | 6.46E−02 | 4.60E−03 | 1.38E−01 |
| CHRND | NM_001256657 | 2.19E−01 | 7.80E−03 | 4.90E−02 | 1.01E−02 | 2.27E−02 | 3.81E−02 |
| MEPE | NM_001291183 | 9.15E−02 | 1.39E−02 | 5.30E−02 | 9.63E−02 | 1.95E−02 | 7.58E−02 |
| REM1 | NM_014012 | 1.64E−01 | 2.03E−02 | 3.23E−02 | 2.88E−02 | 1.16E−02 | 9.36E−02 |
| RPUSD4 | NM_032795 | 4.70E−03 | 3.67E−02 | 9.50E−03 | 1.02E−01 | 3.07E−02 | 1.67E−01 |
| RPS11 | NM_001015 | 8.68E−02 | 7.76E−03 | 1.05E−01 | 2.11E−02 | 9.78E−02 | 3.68E−02 |
| VAMP2 | NM_014232 | 4.60E−02 | 4.00E−02 | 1.89E−02 | 9.93E−02 | 2.01E−02 | 1.31E−01 |
| DUSP10 | NM_144728 | 1.37E−01 | 9.08E−02 | 6.90E−02 | 1.22E−02 | 1.38E−02 | 3.26E−02 |
| NSG1 | NM_001287763 | 4.81E−03 | 8.65E−02 | 2.21E−03 | 7.11E−02 | 1.58E−01 | 3.38E−02 |
| CDKN2A | NM_000077 | 2.52E−02 | 1.32E−02 | 3.08E−02 | 4.49E−02 | 2.29E−01 | 1.36E−02 |
| MBD3L3 | NM_001164425 | 3.71E−03 | 6.80E−02 | 1.18E−02 | 1.06E−01 | 7.68E−02 | 9.45E−02 |
| CCDC109B | NM_017918 | 1.24E−01 | 1.34E−01 | 1.59E−02 | 3.74E−02 | 5.98E−03 | 4.40E−02 |
| CMTM4 | NM_181521 | 9.98E−02 | 1.52E−02 | 1.90E−02 | 5.62E−02 | 5.71E−02 | 1.16E−01 |
| PLA2G4B | NM_001114633 | 2.10E−02 | 1.64E−01 | 4.71E−02 | 5.11E−02 | 4.40E−02 | 3.60E−02 |
| SRC | NM_198291 | 1.77E−01 | 8.69E−02 | 4.50E−03 | 3.67E−02 | 1.23E−03 | 5.80E−02 |
| WBP1L | NM_017787 | 3.36E−02 | 6.43E−02 | 1.27E−02 | 4.52E−02 | 1.99E−01 | 1.18E−02 |
| SCN11A | NM_014139 | 1.36E−01 | 1.80E−03 | 1.90E−02 | 1.03E−01 | 6.31E−02 | 5.15E−02 |
| LHX2 | NM_004789 | 2.05E−01 | 1.03E−02 | 6.41E−02 | 3.68E−02 | 5.06E−02 | 7.82E−03 |
| DISP2 | NM_033510 | 1.41E−02 | 2.22E−01 | 1.18E−02 | 4.27E−02 | 3.14E−03 | 8.52E−02 |
| PLEC | NM_201382 | 3.26E−02 | 1.45E−01 | 8.27E−02 | 3.35E−02 | 4.98E−02 | 4.09E−02 |
| SHROOM3 | NM_020859 | 6.29E−03 | 1.98E−01 | 4.42E−03 | 6.15E−02 | 5.90E−03 | 1.10E−01 |
| TCF7L2 | NM_001198529 | 6.63E−02 | 1.36E−01 | 2.37E−02 | 6.47E−02 | 7.49E−03 | 9.57E−02 |
| TMPRSS5 | NM_030770 | 7.17E−02 | 5.46E−02 | 7.16E−03 | 8.56E−02 | 8.48E−03 | 1.68E−01 |
| ANKEF1 | NM_198798 | 1.02E−01 | 2.43E−01 | 3.83E−03 | 1.26E−02 | 6.62E−03 | 3.36E−02 |
| CALD1 | NM_033140 | 1.40E−02 | 3.43E−02 | 4.35E−02 | 1.58E−01 | 4.24E−02 | 1.11E−01 |
| NKX2-2 | NM_002509 | 1.10E−01 | 1.18E−01 | 2.65E−02 | 2.82E−02 | 1.57E−02 | 1.05E−01 |
| RPL15 | NM_001253384 | 1.25E−01 | 1.25E−02 | 6.18E−02 | 3.54E−03 | 2.03E−01 | 2.28E−03 |
| TMEM55B | NM_144568 | 7.24E−02 | 1.81E−02 | 8.17E−02 | 6.26E−02 | 8.91E−02 | 8.51E−02 |
| STYX | NM_001130701 | 3.87E−02 | 5.08E−03 | 1.18E−01 | 7.94E−02 | 1.02E−01 | 6.72E−02 |
| EIF2B2 | NM_014239 | 9.31E−02 | 1.06E−01 | 3.73E−02 | 6.70E−02 | 4.50E−02 | 6.27E−02 |
| GOLGA8M | NM_001282468 | 1.35E−01 | 8.66E−02 | 1.20E−01 | 1.50E−02 | 5.17E−02 | 7.57E−03 |
| SMIM12 | NM_001164825 | 8.83E−02 | 1.60E−01 | 6.06E−02 | 6.55E−03 | 7.98E−02 | 1.98E−02 |
| CYB5R3 | NM_001171661 | 1.15E−01 | 7.58E−02 | 4.22E−02 | 6.28E−02 | 6.98E−02 | 5.15E−02 |
| POP1 | NM_015029 | 1.52E−01 | 3.10E−02 | 6.56E−02 | 3.57E−02 | 9.83E−02 | 3.48E−02 |
| PRAC1 | NM_032391 | 2.44E−02 | 5.20E−02 | 6.53E−02 | 3.57E−02 | 1.73E−01 | 7.05E−02 |
| LTN1 | NM_015565 | 6.29E−02 | 9.84E−02 | 6.78E−02 | 8.46E−02 | 2.92E−02 | 8.14E−02 |
| ABCA4 | NM_000350 | 2.42E−02 | 2.82E−02 | 1.59E−01 | 1.04E−01 | 7.19E−02 | 4.14E−02 |
| DAO | NM_001917 | 5.74E−02 | 1.51E−01 | 3.70E−02 | 3.59E−02 | 4.52E−02 | 1.04E−01 |
| FAM47A | NM_203408 | 4.28E−02 | 2.43E−01 | 3.07E−02 | 3.38E−02 | 3.07E−02 | 5.05E−02 |
| TMTC2 | NM_152588 | 6.66E−02 | 6.90E−02 | 1.07E−01 | 5.70E−02 | 8.13E−02 | 5.11E−02 |
| ALAS1 | NM_199166 | 1.04E−01 | 2.47E−02 | 1.28E−01 | 3.73E−02 | 1.22E−01 | 1.78E−02 |
| LIN7C | NM_018362 | 1.84E−01 | 3.79E−02 | 9.41E−02 | 2.21E−02 | 4.86E−02 | 4.60E−02 |
| C3orf80 | NM_001168214 | 9.04E−02 | 4.85E−02 | 4.06E−02 | 1.08E−01 | 4.02E−02 | 1.06E−01 |
| TRIM50 | NM_001281451 | 1.13E−02 | 8.34E−02 | 3.13E−02 | 3.80E−02 | 1.86E−01 | 8.65E−02 |
| CMTM3 | NM_144601 | 1.87E−01 | 1.93E−02 | 8.37E−02 | 5.90E−02 | 8.67E−02 | 1.08E−03 |
| LEMD2 | NM_181336 | 2.30E−01 | 3.31E−02 | 1.66E−02 | 3.94E−02 | 3.39E−03 | 1.15E−01 |
| DPM3 | NM_153741 | 1.77E−01 | 1.67E−02 | 1.34E−01 | 2.83E−03 | 1.13E−01 | 2.55E−03 |
| TRIM7 | NM_203293 | 1.07E−01 | 3.00E−03 | 9.13E−02 | 7.56E−03 | 2.16E−01 | 2.01E−02 |
| YY1AP1 | NM_001198902 | 2.57E−01 | 5.07E−03 | 1.02E−01 | 1.87E−02 | 4.21E−02 | 2.13E−02 |
| KCNV1 | NM_014379 | 7.28E−02 | 1.64E−02 | 1.54E−01 | 3.94E−02 | 6.25E−02 | 1.02E−01 |
| NDRG4 | NM_022910 | 1.93E−02 | 2.33E−01 | 5.20E−02 | 4.00E−02 | 2.72E−02 | 7.61E−02 |
| SH3TC1 | NM_018986 | 6.37E−02 | 1.80E−01 | 8.64E−03 | 1.89E−02 | 8.74E−03 | 1.68E−01 |
| LITAF | NM_004862 | 1.26E−02 | 3.03E−02 | 8.15E−02 | 3.83E−02 | 2.38E−01 | 4.82E−02 |
| CD109 | NM_001159587 | 1.74E−01 | 5.71E−03 | 1.27E−01 | 1.55E−02 | 1.12E−01 | 1.52E−02 |
| ATG16L2 | NM_033388 | 1.20E−01 | 2.26E−02 | 3.82E−02 | 1.08E−01 | 9.74E−02 | 6.42E−02 |
| FAM151B | NM_205548 | 2.39E−02 | 1.21E−01 | 7.67E−02 | 8.04E−02 | 6.46E−02 | 8.65E−02 |
| ANK1 | NM_020475 | 1.93E−01 | 2.30E−02 | 7.85E−02 | 5.01E−03 | 4.64E−02 | 1.08E−01 |
| ATF7IP | NM_018179 | 2.47E−02 | 6.73E−02 | 5.40E−02 | 7.62E−02 | 4.26E−02 | 1.90E−01 |
| PLAA | NM_001031689 | 8.70E−03 | 2.32E−01 | 2.74E−02 | 8.10E−02 | 1.94E−02 | 8.80E−02 |
| FCER1A | NM_002001 | 1.55E−02 | 1.44E−01 | 7.49E−02 | 8.75E−02 | 3.79E−02 | 9.79E−02 |
| HGC6.3 | NM_001129895 | 2.18E−01 | 3.68E−02 | 4.06E−02 | 3.08E−02 | 9.85E−03 | 1.24E−01 |
| EXOC4 | NM_021807 | 9.51E−02 | 1.02E−01 | 2.05E−02 | 1.50E−01 | 7.55E−02 | 2.08E−02 |
| CHD3 | NM_001005271 | 9.99E−02 | 6.73E−02 | 7.89E−02 | 4.29E−02 | 2.81E−02 | 1.50E−01 |
| LZTR1 | NM_006767 | 2.06E−01 | 9.78E−02 | 4.92E−02 | 3.29E−02 | 2.07E−02 | 6.06E−02 |
| ZIC5 | NM_033132 | 2.39E−02 | 1.78E−01 | 7.86E−03 | 9.17E−02 | 1.18E−01 | 5.18E−02 |
| EFCAB1 | NM_001142857 | 3.24E−02 | 8.53E−02 | 1.31E−01 | 5.16E−02 | 1.06E−01 | 6.63E−02 |
| TPRN | NM_001128228 | 1.65E−01 | 2.14E−02 | 1.46E−01 | 4.09E−03 | 1.20E−01 | 1.67E−02 |
| LRFN1 | NM_020862 | 7.98E−02 | 8.76E−02 | 4.17E−02 | 9.53E−02 | 1.60E−01 | 8.76E−03 |
| NSL1 | NM_015471 | 1.45E−02 | 2.09E−01 | 2.80E−02 | 1.55E−01 | 1.54E−02 | 5.16E−02 |
| CELF2 | NM_006561 | 3.83E−03 | 2.23E−01 | 1.73E−02 | 9.70E−02 | 4.68E−02 | 8.65E−02 |
| GHRHR | NM_000823 | 6.90E−02 | 2.00E−02 | 4.26E−02 | 1.38E−01 | 8.03E−02 | 1.25E−01 |
| FZR1 | NM_016263 | 5.21E−02 | 5.89E−02 | 3.08E−01 | 8.41E−04 | 3.50E−02 | 2.13E−02 |
| SERF2 | NM_001199877 | 1.57E−02 | 1.00E−01 | 1.11E−03 | 1.41E−01 | 9.08E−02 | 1.28E−01 |
| ZEB1 | NM_001174096 | 1.84E−01 | 1.58E−02 | 5.26E−02 | 7.88E−02 | 2.30E−02 | 1.25E−01 |
| PPP3R2 | NM_147180 | 3.10E−01 | 2.97E−02 | 8.74E−02 | 2.91E−03 | 3.88E−02 | 1.07E−02 |
| CST4 | NM_001899 | 1.62E−01 | 1.76E−03 | 1.94E−01 | 8.45E−04 | 1.23E−01 | 2.74E−03 |
| PCYT2 | NM_001184917 | 1.44E−02 | 4.53E−02 | 1.12E−02 | 2.14E−01 | 2.10E−02 | 1.79E−01 |
| DCN | NM_001920 | 2.37E−01 | 4.42E−04 | 1.33E−01 | 7.49E−03 | 9.65E−02 | 1.11E−02 |
| KRAS | NM_004985 | 7.54E−02 | 8.06E−04 | 1.98E−01 | 3.86E−03 | 1.97E−01 | 1.05E−02 |
| KCNMA1 | NM_001271518 | 2.86E−01 | 3.07E−02 | 1.03E−01 | 4.89E−03 | 4.76E−02 | 1.37E−02 |
| SMARCA2 | NM_001289397 | 3.79E−02 | 3.06E−02 | 1.32E−02 | 9.24E−02 | 3.23E−02 | 2.81E−01 |
| SSX4B | NM_001034832 | 6.97E−02 | 1.54E−01 | 6.85E−02 | 7.26E−02 | 6.16E−02 | 6.11E−02 |
| LDLRAD3 | NM_174902 | 2.10E−01 | 4.20E−03 | 2.40E−02 | 5.47E−02 | 1.10E−01 | 8.58E−02 |
| RPL6 | NM_000970 | 1.13E−01 | 2.30E−03 | 1.98E−01 | 4.85E−03 | 1.66E−01 | 5.56E−03 |
| FLI1 | NM_001167681 | 2.22E−01 | 1.82E−02 | 2.78E−02 | 8.26E−02 | 1.72E−02 | 1.22E−01 |
| KCNQ4 | NM_004700 | 7.37E−02 | 6.00E−02 | 1.11E−01 | 3.55E−02 | 8.71E−02 | 1.25E−01 |
| BCL11B | NM_138576 | 2.55E−02 | 9.14E−02 | 1.21E−01 | 8.58E−02 | 7.69E−02 | 9.32E−02 |
| ATP7A | NM_000052 | 1.30E−01 | 5.79E−02 | 5.93E−02 | 1.51E−01 | 6.61E−02 | 3.04E−02 |
| CACNA1B | NM_001243812 | 1.12E−01 | 8.87E−02 | 1.22E−01 | 3.13E−02 | 1.16E−01 | 2.54E−02 |
| COL11A1 | NM_001190709 | 1.35E−01 | 9.37E−02 | 3.62E−02 | 7.31E−02 | 1.77E−02 | 1.40E−01 |
| SSH3 | NM_017857 | 8.47E−02 | 5.71E−02 | 3.34E−02 | 7.82E−02 | 5.77E−02 | 1.85E−01 |
| LOC113230 | NM_001291291 | 1.28E−01 | 3.69E−02 | 7.80E−02 | 4.71E−02 | 2.07E−01 | 1.49E−03 |
| PLA2G7 | NM_001168357 | 1.64E−01 | 1.40E−01 | 3.91E−02 | 1.81E−02 | 2.75E−02 | 1.11E−01 |
| CACNA2D2 | NM_001174051 | 3.02E−02 | 9.41E−02 | 4.65E−02 | 4.29E−02 | 1.74E−01 | 1.12E−01 |
| UBE2T | NM_014176 | 1.29E−01 | 1.08E−02 | 5.16E−02 | 3.87E−02 | 2.38E−01 | 3.45E−02 |
| SPTBN2 | NM_006946 | 9.07E−02 | 1.73E−01 | 4.18E−02 | 8.61E−02 | 4.38E−02 | 6.83E−02 |
| SERPINB8 | NM_001276490 | 1.02E−01 | 2.00E−03 | 1.25E−01 | 4.86E−02 | 1.99E−01 | 2.81E−02 |
| TMEM182 | NM_144632 | 7.59E−02 | 6.12E−02 | 1.61E−01 | 4.61E−02 | 1.06E−01 | 5.43E−02 |
| EFHC1 | NM_001172420 | 6.57E−02 | 3.72E−03 | 1.07E−01 | 1.54E−02 | 3.00E−01 | 1.36E−02 |
| KIF13A | NM_001105568 | 2.01E−01 | 2.33E−02 | 2.90E−02 | 1.23E−01 | 7.24E−02 | 5.99E−02 |
| SOCS2 | NM_001270467 | 2.34E−01 | 7.11E−02 | 5.05E−02 | 5.28E−02 | 3.15E−02 | 6.87E−02 |
| GCNT2 | NM_145649 | 1.26E−02 | 1.45E−01 | 2.15E−02 | 9.20E−02 | 1.66E−01 | 7.30E−02 |
| PHKB | NM_000293 | 7.41E−02 | 1.70E−01 | 6.83E−02 | 6.07E−02 | 4.97E−02 | 8.87E−02 |
| ABI1 | NM_001178116 | 4.80E−02 | 6.97E−02 | 1.09E−01 | 6.43E−02 | 1.20E−01 | 1.06E−01 |
| OCLN | NM_001205255 | 2.53E−02 | 1.32E−01 | 6.92E−02 | 1.02E−01 | 2.89E−02 | 1.59E−01 |
| TRIM5 | NM_033092 | 1.08E−01 | 1.85E−02 | 1.62E−01 | 4.66E−02 | 1.32E−01 | 5.00E−02 |
| NFE2 | NM_001136023 | 3.94E−03 | 1.43E−01 | 2.47E−02 | 1.89E−01 | 2.19E−02 | 1.36E−01 |
| PHKA1 | NM_001172436 | 6.59E−02 | 1.26E−01 | 3.35E−03 | 7.69E−02 | 2.69E−02 | 2.23E−01 |
| OR2C3 | NM_198074 | 3.05E−02 | 2.33E−01 | 1.11E−02 | 1.25E−01 | 1.26E−03 | 1.20E−01 |
| LTBR | NM_002342 | 1.64E−01 | 1.68E−03 | 2.38E−01 | 1.38E−02 | 8.74E−02 | 1.67E−02 |
| TACR3 | NM_001059 | 8.28E−02 | 7.57E−04 | 1.81E−01 | 2.05E−02 | 2.18E−01 | 1.98E−02 |
| PRKRA | NM_001139518 | 2.64E−01 | 9.51E−02 | 3.10E−02 | 1.43E−02 | 8.61E−02 | 3.62E−02 |
| PKIG | NM_001281444 | 9.46E−03 | 9.63E−02 | 3.36E−02 | 2.61E−01 | 6.60E−02 | 6.18E−02 |
| YPEL5 | NM_001127400 | 8.93E−02 | 4.32E−02 | 6.79E−02 | 8.25E−02 | 1.44E−01 | 1.02E−01 |
| TGFBR1 | NM_001130916 | 1.80E−01 | 1.12E−02 | 3.55E−02 | 4.01E−02 | 1.37E−01 | 1.27E−01 |
| UBXN7 | NM_015562 | 4.72E−02 | 3.11E−02 | 1.98E−01 | 2.77E−02 | 1.66E−01 | 6.13E−02 |
| PRDX1 | NM_001202431 | 1.29E−04 | 1.67E−01 | 3.00E−03 | 1.74E−01 | 9.57E−03 | 1.81E−01 |
| KBTBD7 | NM_032138 | 5.53E−02 | 8.49E−02 | 1.41E−01 | 6.99E−02 | 9.21E−02 | 9.24E−02 |
| SHE | NM_001010846 | 8.72E−02 | 1.51E−03 | 6.85E−02 | 7.96E−03 | 3.46E−01 | 2.45E−02 |
| ZNF486 | NM_052852 | 1.45E−01 | 6.22E−02 | 1.98E−01 | 4.14E−04 | 1.29E−01 | 1.81E−03 |
| TTLL10 | NM_001130045 | 1.92E−01 | 5.51E−02 | 3.50E−02 | 7.47E−02 | 9.89E−02 | 8.36E−02 |
| BACE1 | NM_138971 | 1.35E−01 | 1.71E−01 | 6.95E−02 | 3.17E−02 | 9.50E−02 | 3.74E−02 |
| GNG11 | NM_004126 | 1.09E−01 | 6.39E−03 | 2.02E−01 | 1.51E−01 | 4.93E−02 | 2.20E−02 |
| HUS1B | NM_148959 | 9.86E−02 | 7.86E−02 | 1.79E−01 | 1.46E−02 | 1.47E−01 | 2.16E−02 |
| ART1 | NM_004314 | 1.99E−01 | 8.50E−03 | 9.16E−02 | 5.54E−02 | 4.90E−02 | 1.36E−01 |
| MTRR | NM_024010 | 3.98E−02 | 2.86E−02 | 1.40E−02 | 2.38E−01 | 4.06E−02 | 1.79E−01 |
| TLE3 | NM_020908 | 1.53E−01 | 9.97E−03 | 1.71E−01 | 2.87E−03 | 2.01E−01 | 2.80E−03 |
| WARS2 | NM_015836 | 1.80E−03 | 9.17E−02 | 4.42E−04 | 1.22E−01 | 2.17E−03 | 3.23E−01 |
| ADCYAP1 | NM_001099733 | 9.29E−02 | 1.72E−01 | 2.27E−02 | 5.93E−02 | 1.26E−01 | 6.91E−02 |
| FAM155A | NM_001080396 | 2.82E−02 | 9.06E−02 | 8.35E−02 | 1.33E−01 | 3.65E−02 | 1.70E−01 |
| CD151 | NM_139030 | 1.42E−01 | 1.14E−01 | 2.90E−02 | 2.36E−02 | 1.70E−01 | 6.38E−02 |
| KIF3A | NM_007054 | 1.23E−01 | 4.11E−02 | 7.51E−02 | 4.36E−02 | 1.51E−01 | 1.11E−01 |
| CCDC84 | NM_198489 | 1.58E−01 | 4.66E−03 | 1.14E−01 | 5.92E−02 | 1.95E−01 | 1.66E−02 |
| OR4C11 | NM_001004700 | 9.85E−02 | 4.21E−02 | 4.58E−02 | 1.43E−01 | 1.84E−01 | 3.54E−02 |
| ZNF274 | NM_133502 | 1.32E−01 | 2.30E−02 | 1.35E−01 | 3.64E−02 | 1.82E−01 | 4.12E−02 |
| GNG7 | NM_052847 | 7.90E−02 | 1.85E−01 | 6.29E−02 | 6.92E−02 | 3.46E−02 | 1.19E−01 |
| TTC37 | NM_014639 | 5.04E−04 | 9.77E−02 | 4.33E−03 | 7.50E−02 | 8.13E−03 | 3.64E−01 |
| DNPEP | NM_012100 | 1.94E−02 | 7.68E−02 | 3.59E−03 | 1.72E−01 | 5.66E−03 | 2.76E−01 |
| GPRASP2 | NM_001184876 | 2.72E−02 | 9.80E−02 | 1.43E−01 | 2.92E−03 | 2.78E−01 | 4.28E−03 |
| C9orf92 | NM_001271829 | 1.46E−01 | 1.20E−02 | 1.80E−01 | 3.54E−02 | 1.18E−01 | 6.39E−02 |
| MIER1 | NM_001146112 | 1.03E−01 | 1.04E−01 | 7.82E−02 | 8.82E−02 | 7.85E−02 | 1.04E−01 |
| SMIM24 | NM_001136503 | 4.64E−02 | 1.83E−01 | 3.56E−02 | 1.02E−01 | 8.56E−02 | 1.09E−01 |
| CYB5D2 | NM_144611 | 8.35E−02 | 1.55E−01 | 6.73E−02 | 9.37E−02 | 1.82E−02 | 1.45E−01 |
| ABHD8 | NM_024527 | 5.25E−03 | 1.76E−01 | 3.47E−03 | 1.75E−01 | 1.93E−03 | 2.01E−01 |
| MX1 | NM_001178046 | 1.78E−02 | 1.09E−01 | 6.08E−02 | 1.26E−01 | 8.53E−02 | 1.64E−01 |
| TNFAIP8L2- | NM_001204848 | 1.88E−01 | 8.00E−02 | 4.19E−02 | 5.15E−02 | 1.32E−01 | 7.12E−02 |
| SCNM1 | | | | | | | |
| BEX2 | NM_001168401 | 4.71E−02 | 1.25E−01 | 5.16E−02 | 1.03E−01 | 8.09E−02 | 1.58E−01 |
| SLC25A1 | NM_005984 | 8.58E−02 | 1.29E−01 | 8.31E−02 | 8.38E−02 | 3.18E−02 | 1.53E−01 |
| GPS2 | NM_004489 | 9.06E−02 | 2.14E−01 | 6.41E−02 | 6.10E−02 | 2.57E−02 | 1.11E−01 |
| CORO1C | NM_001105237 | 1.01E−01 | 1.62E−02 | 1.60E−01 | 9.07E−02 | 1.63E−01 | 3.69E−02 |
| FMO3 | NM_006894 | 6.90E−02 | 9.04E−02 | 1.77E−01 | 1.59E−02 | 1.98E−01 | 1.75E−02 |
| THOC7 | NM_025075 | 2.68E−01 | 1.10E−01 | 5.04E−02 | 6.45E−02 | 1.03E−02 | 6.48E−02 |
| ENY2 | NM_001193557 | 2.36E−02 | 1.56E−02 | 9.24E−02 | 8.29E−02 | 2.23E−01 | 1.32E−01 |
| LARP6 | NM_001286679 | 1.92E−01 | 1.73E−01 | 3.80E−02 | 1.57E−02 | 1.25E−01 | 2.77E−02 |
| AP1S1 | NM_001283 | 6.46E−02 | 5.32E−02 | 1.00E−01 | 8.52E−02 | 1.13E−01 | 1.58E−01 |
| HAUS4 | NM_001166269 | 3.90E−01 | 4.63E−03 | 6.60E−02 | 3.56E−02 | 8.14E−03 | 6.98E−02 |
| SHD | NM_020209 | 1.43E−03 | 2.01E−01 | 2.16E−03 | 2.02E−01 | 2.72E−03 | 1.64E−01 |
| ADRA2A | NM_000681 | 1.25E−01 | 1.15E−01 | 1.09E−01 | 3.08E−02 | 1.06E−01 | 8.83E−02 |
| NCDN | NM_001014839 | 1.06E−02 | 1.42E−01 | 1.12E−01 | 1.27E−01 | 8.19E−02 | 1.01E−01 |
| ZNF764 | NM_001172679 | 1.92E−01 | 1.02E−02 | 1.68E−01 | 2.09E−02 | 1.34E−01 | 5.48E−02 |
| KIAA0247 | NM_014734 | 1.72E−01 | 3.48E−02 | 2.17E−01 | 2.15E−02 | 1.12E−01 | 2.37E−02 |
| ZNF283 | NM_181845 | 1.23E−01 | 1.18E−01 | 1.52E−01 | 3.17E−02 | 6.05E−02 | 9.80E−02 |
| LSP1 | NM_001013253 | 2.08E−01 | 3.19E−04 | 1.82E−01 | 2.19E−02 | 1.26E−01 | 4.54E−02 |
| ATG16L1 | NM_017974 | 7.74E−02 | 1.33E−01 | 1.09E−01 | 5.09E−02 | 1.71E−01 | 4.31E−02 |
| NOL12 | NM_024313 | 5.42E−02 | 2.18E−02 | 5.00E−02 | 4.98E−02 | 2.72E−01 | 1.40E−01 |
| TMEM132A | NM_017870 | 9.46E−02 | 8.61E−02 | 6.09E−02 | 2.75E−02 | 2.84E−01 | 3.65E−02 |
| MPP2 | NM_001278375 | 1.26E−01 | 5.94E−02 | 2.07E−01 | 1.12E−02 | 1.71E−01 | 1.46E−02 |
| ABCG4 | NM_022169 | 3.61E−03 | 3.51E−02 | 1.27E−01 | 8.69E−02 | 2.02E−01 | 1.36E−01 |
| GSDMD | NM_001166237 | 2.44E−01 | 6.47E−02 | 4.38E−02 | 8.97E−02 | 1.19E−03 | 1.48E−01 |
| OR2D3 | NM_001004684 | 8.35E−02 | 1.53E−01 | 2.63E−02 | 5.89E−02 | 1.20E−01 | 1.54E−01 |
| PLXDC2 | NM_001282736 | 2.23E−01 | 1.47E−01 | 9.95E−02 | 1.44E−02 | 7.48E−02 | 3.84E−02 |
| EGR1 | NM_001964 | 3.63E−02 | 2.08E−01 | 4.68E−02 | 1.24E−01 | 2.54E−02 | 1.58E−01 |
| IMPG2 | NM_016247 | 1.54E−01 | 1.02E−01 | 7.34E−02 | 3.75E−02 | 2.15E−02 | 2.11E−01 |
| C1orf100 | NM_001276349 | 1.59E−01 | 1.29E−01 | 9.03E−02 | 7.89E−02 | 5.59E−02 | 8.74E−02 |
| SLC25A35 | NM_201520 | 3.23E−02 | 1.32E−02 | 1.48E−01 | 1.11E−01 | 2.08E−01 | 9.00E−02 |
| NFIC | NM_205843 | 1.46E−01 | 1.60E−03 | 3.96E−02 | 1.00E−01 | 4.97E−02 | 2.70E−01 |
| HNRNPR | NM_001102398 | 6.11E−02 | 3.86E−02 | 2.01E−01 | 3.20E−02 | 2.26E−01 | 4.78E−02 |
| GALE | NM_001127621 | 1.30E−01 | 2.21E−01 | 9.75E−03 | 5.83E−02 | 5.51E−02 | 1.34E−01 |
| WNT2B | NM_004185 | 1.60E−01 | 1.57E−02 | 5.04E−02 | 2.43E−01 | 1.13E−01 | 2.66E−02 |
| RNF150 | NM_020724 | 2.40E−01 | 1.19E−01 | 3.88E−02 | 1.51E−01 | 2.21E−02 | 3.90E−02 |
| EXOC3L1 | NM_178516 | 3.61E−02 | 4.84E−02 | 1.43E−01 | 1.47E−01 | 8.04E−02 | 1.56E−01 |
| DAPL1 | NM_001017920 | 9.96E−02 | 1.69E−01 | 1.09E−01 | 6.19E−02 | 1.56E−01 | 1.59E−02 |
| MROH6 | NM_001100878 | 1.48E−01 | 1.16E−01 | 6.59E−02 | 9.55E−02 | 1.17E−01 | 6.97E−02 |
| TMEM147 | NM_032635 | 2.50E−04 | 3.55E−01 | 6.74E−03 | 1.04E−01 | 1.27E−01 | 2.03E−02 |
| SLC39A1 | NM_001271960 | 1.09E−01 | 1.28E−01 | 4.71E−02 | 6.91E−02 | 1.53E−01 | 1.07E−01 |
| POLD3 | NM_006591 | 4.85E−03 | 1.46E−01 | 5.09E−03 | 2.75E−01 | 1.16E−01 | 6.60E−02 |
| PHF19 | NM_001286840 | 1.54E−01 | 5.19E−03 | 2.90E−02 | 9.01E−02 | 2.08E−01 | 1.28E−01 |
| IL18RAP | NM_003853 | 1.03E−01 | 2.03E−01 | 1.14E−02 | 1.64E−01 | 1.17E−02 | 1.21E−01 |
| CLTCL1 | NM_001835 | 8.96E−02 | 1.08E−01 | 9.33E−02 | 6.73E−02 | 4.49E−02 | 2.12E−01 |
| NDUFA5 | NM_001282419 | 8.61E−02 | 3.83E−01 | 1.42E−02 | 7.55E−02 | 3.25E−02 | 2.59E−02 |
| RIMKLB | NM_020734 | 2.48E−02 | 3.94E−03 | 2.75E−01 | 3.37E−03 | 3.04E−01 | 6.65E−03 |
| NMT2 | NM_004808 | 3.22E−02 | 2.51E−01 | 1.80E−02 | 1.90E−01 | 1.25E−02 | 1.15E−01 |
| DEDD | NM_001039711 | 3.94E−02 | 2.07E−01 | 2.74E−02 | 1.36E−01 | 7.45E−02 | 1.36E−01 |
| RAI14 | NM_001145520 | 8.76E−02 | 2.18E−01 | 4.79E−02 | 9.74E−02 | 5.23E−02 | 1.17E−01 |
| HIST1H3G | NM_003534 | 1.45E−01 | 1.09E−01 | 1.37E−01 | 3.76E−02 | 1.25E−01 | 6.51E−02 |
| MANEA | NM_024641 | 1.93E−04 | 1.86E−01 | 6.86E−04 | 1.85E−01 | 8.61E−04 | 2.47E−01 |
| PLAG1 | NM_001114635 | 5.82E−02 | 4.66E−01 | 1.02E−02 | 2.78E−02 | 1.06E−03 | 5.68E−02 |
| LDB3 | NM_001171610 | 5.32E−02 | 2.10E−01 | 1.17E−01 | 1.29E−01 | 3.67E−02 | 7.40E−02 |
| DUPD1 | NM_001003892 | 1.85E−01 | 5.19E−03 | 2.11E−01 | 6.19E−03 | 2.13E−01 | 1.23E−03 |
| RHPN1 | NM_052924 | 5.69E−02 | 6.23E−02 | 2.66E−02 | 1.66E−01 | 4.79E−02 | 2.62E−01 |
| ARHGEF26 | NM_001251963 | 2.11E−01 | 4.70E−02 | 1.84E−01 | 1.94E−02 | 1.45E−01 | 1.70E−02 |
| LTF | NM_001199149 | 3.88E−02 | 7.38E−02 | 2.01E−02 | 3.01E−01 | 1.19E−01 | 7.17E−02 |
| C11orf83 | NM_001085372 | 2.50E−01 | 3.42E−02 | 1.55E−01 | 4.56E−02 | 7.57E−02 | 6.49E−02 |
| ITGB1BP2 | NM_012278 | 4.47E−02 | 1.22E−01 | 7.88E−02 | 5.86E−02 | 2.26E−01 | 9.57E−02 |
| RFPL2 | NM_001098527 | 6.13E−02 | 3.42E−01 | 2.24E−02 | 1.16E−01 | 2.70E−02 | 5.68E−02 |
| IQCF2 | NM_203424 | 1.40E−01 | 1.92E−02 | 1.66E−01 | 6.33E−02 | 1.45E−01 | 9.42E−02 |
| SRP54 | NM_003136 | 1.73E−01 | 5.95E−02 | 8.64E−03 | 1.38E−01 | 7.09E−02 | 1.80E−01 |
| SLURP1 | NM_020427 | 1.19E−01 | 6.65E−03 | 6.83E−02 | 7.16E−02 | 2.51E−01 | 1.14E−01 |
| TAF3 | NM_031923 | 2.25E−01 | 1.51E−01 | 1.06E−02 | 1.08E−01 | 1.93E−04 | 1.36E−01 |
| MFSD5 | NM_001170790 | 2.44E−01 | 4.46E−02 | 7.85E−02 | 9.56E−03 | 2.28E−01 | 2.62E−02 |
| TVP23B | NM_016078 | 1.67E−02 | 7.55E−02 | 2.01E−02 | 2.38E−01 | 3.02E−02 | 2.51E−01 |
| EEFSEC | NM_021937 | 8.61E−02 | 7.49E−02 | 7.48E−02 | 5.21E−02 | 2.54E−01 | 8.99E−02 |
| IFIT3 | NM_001549 | 8.60E−02 | 1.56E−01 | 3.90E−02 | 1.05E−01 | 1.35E−01 | 1.12E−01 |
| CCNT2 | NM_058241 | 1.65E−01 | 1.09E−02 | 7.17E−02 | 2.22E−01 | 8.46E−02 | 7.89E−02 |
| OBP2A | NM_014582 | 3.17E−02 | 9.45E−02 | 1.71E−02 | 1.89E−01 | 1.22E−01 | 1.81E−01 |
| ZBTB22 | NM_001145338 | 2.41E−01 | 3.23E−03 | 1.89E−01 | 3.51E−02 | 1.14E−01 | 5.22E−02 |
| LRP10 | NM_014045 | 9.12E−02 | 2.85E−02 | 5.29E−02 | 9.29E−02 | 1.55E−01 | 2.18E−01 |
| CSAG1 | NM_153478 | 3.74E−02 | 2.10E−01 | 1.03E−01 | 1.59E−01 | 1.17E−01 | 1.21E−02 |
| AICDA | NM_020661 | 3.58E−02 | 1.17E−01 | 1.05E−01 | 2.41E−01 | 6.67E−02 | 7.50E−02 |
| PDK2 | NM_001199900 | 7.22E−03 | 2.42E−02 | 1.20E−01 | 1.05E−01 | 3.17E−01 | 6.70E−02 |
| ASB8 | NM_024095 | 6.92E−02 | 6.20E−02 | 1.45E−02 | 2.72E−02 | 4.28E−01 | 4.08E−02 |
| ZNF277 | NM_021994 | 1.89E−02 | 2.90E−01 | 1.64E−02 | 4.41E−02 | 1.94E−01 | 7.82E−02 |
| RAB2A | NM_002865 | 1.18E−01 | 1.39E−01 | 4.63E−02 | 1.30E−01 | 7.07E−02 | 1.38E−01 |
| C10orf105 | NM_001164375 | 9.48E−02 | 1.49E−01 | 1.69E−01 | 4.06E−02 | 6.47E−02 | 1.26E−01 |
| CCDC169 | NM_001144984 | 3.14E−02 | 1.04E−01 | 8.82E−02 | 5.74E−02 | 2.68E−01 | 9.39E−02 |
| XPNPEP1 | NM_001167604 | 4.03E−04 | 2.50E−01 | 2.68E−02 | 1.33E−01 | 1.77E−02 | 2.16E−01 |
| MXRA8 | NM_001282582 | 1.23E−01 | 5.65E−02 | 9.69E−02 | 2.76E−01 | 4.99E−02 | 4.23E−02 |
| TOX2 | NM_001098797 | 1.44E−01 | 1.04E−01 | 5.94E−02 | 2.71E−02 | 2.18E−01 | 9.15E−02 |
| TEC | NM_003215 | 2.85E−03 | 9.17E−02 | 1.48E−01 | 7.72E−02 | 3.00E−01 | 2.60E−02 |
| SYNGR4 | NM_012451 | 6.43E−02 | 2.85E−01 | 7.27E−02 | 8.27E−02 | 2.81E−02 | 1.14E−01 |
| DYNC1I1 | NM_001135556 | 2.69E−01 | 1.18E−01 | 7.84E−02 | 7.05E−02 | 2.40E−02 | 9.06E−02 |
| DEXI | NM_014015 | 1.35E−01 | 2.78E−02 | 7.55E−02 | 4.15E−02 | 3.01E−01 | 6.94E−02 |
| PLAC1 | NM_021796 | 1.03E−01 | 5.50E−02 | 1.70E−01 | 5.25E−02 | 2.26E−01 | 4.41E−02 |
| PNOC | NM_001284244 | 3.39E−02 | 5.90E−02 | 2.34E−02 | 3.35E−01 | 2.09E−03 | 1.99E−01 |
| ZNF827 | NM_178835 | 6.82E−03 | 5.15E−02 | 4.18E−01 | 7.71E−03 | 1.58E−01 | 1.06E−02 |
| UIMC1 | NM_001199297 | 3.72E−01 | 1.89E−01 | 5.10E−02 | 1.73E−02 | 1.24E−02 | 1.31E−02 |
| TAAR8 | NM_053278 | 9.58E−02 | 1.77E−02 | 9.83E−02 | 4.20E−02 | 3.51E−01 | 5.00E−02 |
| HNRNPF | NM_004966 | 1.83E−01 | 1.63E−02 | 1.86E−01 | 5.42E−02 | 9.01E−02 | 1.25E−01 |
| NREP | NM_001142483 | 2.19E−01 | 1.97E−01 | 1.27E−02 | 8.78E−02 | 9.52E−03 | 1.29E−01 |
| ZNF605 | NM_001164715 | 1.28E−02 | 2.53E−01 | 3.07E−02 | 1.10E−01 | 5.18E−02 | 1.98E−01 |
| SHISA5 | NM_001272066 | 7.00E−02 | 1.72E−01 | 1.15E−01 | 6.73E−02 | 1.25E−01 | 1.08E−01 |
| MCL1 | NM_021960 | 3.22E−04 | 1.45E−01 | 2.52E−02 | 2.21E−01 | 5.20E−02 | 2.14E−01 |
| TRIM62 | NM_018207 | 7.03E−02 | 1.71E−01 | 5.25E−02 | 1.19E−01 | 5.37E−02 | 1.91E−01 |
| C9orf57 | NM_001128618 | 5.67E−02 | 1.70E−01 | 7.74E−02 | 1.50E−01 | 9.52E−02 | 1.09E−01 |
| RLTPR | NM_001013838 | 1.18E−02 | 1.33E−01 | 8.16E−02 | 1.01E−01 | 7.14E−02 | 2.60E−01 |
| RPL13 | NM_001243130 | 2.08E−01 | 9.52E−02 | 6.92E−02 | 9.65E−02 | 6.13E−02 | 1.30E−01 |
| RNF170 | NM_001160224 | 3.02E−01 | 1.44E−01 | 7.99E−02 | 2.54E−02 | 2.84E−02 | 8.06E−02 |
| TLX2 | NM_016170 | 5.01E−02 | 1.38E−01 | 7.09E−02 | 2.27E−02 | 2.98E−01 | 8.31E−02 |
| ZBTB49 | NM_145291 | 1.87E−01 | 1.13E−01 | 3.84E−02 | 1.14E−01 | 7.81E−02 | 1.33E−01 |
| MEX3A | NM_001093725 | 1.50E−01 | 1.08E−01 | 6.88E−02 | 4.38E−02 | 2.06E−01 | 8.71E−02 |
| TERF2 | NM_005652 | 4.13E−01 | 6.27E−02 | 5.07E−02 | 1.33E−02 | 1.11E−01 | 1.60E−02 |
| SLC8A2 | NM_015063 | 1.98E−02 | 8.50E−02 | 1.28E−01 | 8.70E−02 | 2.19E−01 | 1.28E−01 |
| FBXO33 | NM_203301 | 7.15E−02 | 6.71E−02 | 7.87E−02 | 1.29E−01 | 2.90E−01 | 3.13E−02 |
| NLGN4Y | NM_014893 | 1.46E−01 | 2.06E−01 | 4.46E−02 | 1.86E−01 | 5.50E−02 | 2.95E−02 |
| PRDM8 | NM_020226 | 1.54E−02 | 3.56E−01 | 1.64E−02 | 4.11E−02 | 7.35E−02 | 1.65E−01 |
| C9orf84 | NM_173521 | 1.75E−02 | 1.73E−01 | 7.81E−02 | 1.59E−01 | 2.10E−01 | 3.21E−02 |
| MRPS15 | NM_031280 | 5.91E−02 | 2.43E−01 | 8.17E−02 | 1.06E−01 | 7.32E−02 | 1.08E−01 |
| AHCYL1 | NM_001242674 | 1.33E−01 | 1.86E−01 | 4.62E−02 | 1.21E−01 | 3.08E−02 | 1.56E−01 |
| ZNF766 | NM_001010851 | 2.49E−01 | 1.54E−02 | 2.04E−01 | 6.52E−03 | 1.89E−01 | 7.56E−03 |
| LOXL1 | NM_005576 | 7.10E−02 | 2.44E−01 | 1.36E−01 | 4.04E−02 | 1.17E−01 | 6.37E−02 |
| PRMT7 | NM_001290018 | 8.67E−02 | 3.01E−01 | 6.80E−02 | 1.18E−01 | 1.52E−03 | 9.86E−02 |
| ELP3 | NM_018091 | 7.87E−02 | 2.65E−01 | 3.11E−03 | 7.88E−02 | 1.40E−01 | 1.08E−01 |
| CNN3 | NM_001839 | 6.82E−02 | 5.83E−02 | 1.45E−01 | 1.24E−01 | 1.53E−01 | 1.25E−01 |
| NPY5R | NM_006174 | 2.34E−02 | 1.68E−02 | 3.39E−02 | 2.72E−01 | 2.33E−02 | 3.07E−01 |
| SLITRK1 | NM_052910 | 1.12E−01 | 1.92E−01 | 2.31E−01 | 6.37E−02 | 5.25E−02 | 2.79E−02 |
| TEAD4 | NM_003213 | 7.93E−03 | 3.42E−01 | 5.19E−03 | 1.26E−01 | 1.65E−03 | 1.98E−01 |
| THOP1 | NM_003249 | 2.46E−01 | 1.70E−01 | 1.78E−01 | 1.85E−02 | 4.93E−02 | 1.88E−02 |
| GSN | NM_001258030 | 1.96E−01 | 5.48E−02 | 1.34E−01 | 8.68E−02 | 9.13E−02 | 1.18E−01 |
| APRT | NM_000485 | 4.29E−02 | 1.67E−01 | 1.23E−01 | 9.02E−02 | 9.38E−02 | 1.65E−01 |
| CORO6 | NM_032854 | 1.15E−01 | 4.90E−02 | 5.24E−02 | 1.35E−01 | 5.12E−02 | 2.80E−01 |
| COL9A3 | NM_001853 | 2.43E−02 | 6.13E−02 | 9.70E−02 | 8.18E−02 | 2.97E−01 | 1.21E−01 |
| MAGEB17 | NM_001277307 | 7.71E−02 | 2.27E−01 | 9.85E−02 | 1.73E−01 | 8.19E−02 | 2.58E−02 |
| CD97 | NM_078481 | 2.31E−02 | 4.32E−03 | 1.27E−01 | 1.19E−01 | 2.20E−01 | 1.90E−01 |
| PPM1G | NM_177983 | 3.43E−02 | 2.16E−01 | 6.45E−02 | 1.53E−01 | 6.95E−02 | 1.46E−01 |
| DVL3 | NM_004423 | 7.26E−02 | 3.83E−02 | 2.28E−01 | 5.13E−02 | 2.55E−01 | 3.99E−02 |
| CREG2 | NM_153836 | 4.68E−01 | 4.04E−02 | 1.13E−01 | 3.78E−03 | 5.29E−02 | 7.79E−03 |
| DMPK | NM_001081563 | 8.29E−02 | 2.32E−02 | 9.12E−02 | 1.01E−01 | 6.48E−02 | 3.23E−01 |
| EIF2B4 | NM_172195 | 1.05E−01 | 2.72E−01 | 1.54E−01 | 2.38E−02 | 7.85E−02 | 5.25E−02 |
| PRSS56 | NM_001195129 | 2.61E−01 | 1.20E−01 | 1.35E−01 | 8.06E−02 | 6.25E−02 | 2.75E−02 |
| UAP1L1 | NM_207309 | 7.10E−02 | 1.03E−01 | 2.27E−02 | 1.45E−01 | 1.94E−02 | 3.26E−01 |
| AKAP6 | NM_004274 | 2.01E−01 | 1.44E−01 | 1.44E−01 | 7.78E−02 | 5.61E−02 | 6.41E−02 |
| ZIC1 | NM_003412 | 9.20E−02 | 4.21E−01 | 5.71E−03 | 4.98E−02 | 7.07E−02 | 4.94E−02 |
| DDX43 | NM_018665 | 8.34E−03 | 6.94E−03 | 1.75E−02 | 1.97E−01 | 2.41E−01 | 2.19E−01 |
| NAE1 | NM_003905 | 2.36E−01 | 6.32E−02 | 1.06E−01 | 3.52E−02 | 1.82E−01 | 6.76E−02 |
| PLS1 | NM_002670 | 5.05E−02 | 2.19E−01 | 1.36E−01 | 1.16E−01 | 1.13E−01 | 5.53E−02 |
| TBP | NM_001172085 | 6.20E−02 | 8.95E−02 | 5.81E−02 | 4.36E−02 | 3.23E−01 | 1.16E−01 |
| FAM217A | NM_173563 | 2.33E−01 | 1.59E−02 | 2.34E−01 | 1.09E−01 | 9.60E−02 | 4.64E−03 |
| EXOC3L2 | NM_138568 | 1.67E−01 | 7.14E−02 | 1.41E−01 | 2.83E−02 | 1.37E−01 | 1.49E−01 |
| SOCS3 | NM_003955 | 1.76E−01 | 1.90E−02 | 1.18E−01 | 2.05E−01 | 7.19E−02 | 1.04E−01 |
| TRIM60 | NM_001258025 | 3.66E−01 | 1.65E−02 | 8.02E−02 | 1.21E−02 | 1.96E−01 | 2.31E−02 |
| SAMD8 | NM_144660 | 1.34E−01 | 1.66E−02 | 6.35E−02 | 1.51E−01 | 3.10E−01 | 1.95E−02 |
| SMPDL3B | NM_001009568 | 5.65E−02 | 1.24E−01 | 1.52E−01 | 2.19E−01 | 1.04E−01 | 4.18E−02 |
| FOXP1 | NM_001244808 | 2.00E−02 | 2.82E−01 | 6.04E−02 | 1.55E−01 | 6.84E−02 | 1.12E−01 |
| CHD4 | NM_001273 | 4.63E−01 | 4.45E−02 | 4.18E−02 | 3.64E−02 | 6.41E−03 | 1.06E−01 |
| ELMO1 | NM_014800 | 2.26E−01 | 1.21E−01 | 5.50E−02 | 1.19E−01 | 3.64E−02 | 1.43E−01 |
| PLEKHM3 | NM_001080475 | 3.19E−01 | 1.66E−02 | 1.00E−01 | 3.82E−03 | 2.53E−01 | 8.42E−03 |
| MOB4 | NM_015387 | 2.57E−01 | 4.15E−02 | 1.28E−01 | 3.61E−02 | 2.29E−01 | 1.27E−02 |
| C6orf15 | NM_014070 | 6.86E−04 | 3.84E−01 | 4.91E−02 | 8.89E−02 | 1.03E−01 | 8.17E−02 |
| RANBP1 | NM_001278641 | 1.44E−01 | 2.90E−02 | 2.29E−01 | 9.21E−03 | 2.83E−01 | 1.39E−02 |
| TDRD9 | NM_153046 | 2.59E−01 | 7.23E−02 | 1.25E−01 | 7.99E−02 | 2.58E−02 | 1.48E−01 |
| ATP6V0C | NM_001694 | 5.73E−02 | 9.29E−03 | 2.55E−01 | 3.86E−02 | 2.55E−01 | 9.59E−02 |
| TMPRSS9 | NM_182973 | 6.13E−02 | 9.91E−02 | 1.34E−01 | 1.42E−01 | 1.49E−01 | 1.26E−01 |
| ZNF408 | NM_001184751 | 4.00E−02 | 9.87E−02 | 3.82E−02 | 2.17E−01 | 1.50E−01 | 1.67E−01 |
| ZNF648 | NM_001009992 | 8.06E−02 | 1.82E−01 | 6.02E−02 | 2.18E−01 | 2.95E−02 | 1.42E−01 |
| USP22 | NM_015276 | 4.08E−01 | 1.42E−01 | 7.96E−02 | 2.13E−02 | 1.97E−02 | 4.32E−02 |
| PCDP1 | NM_001271049 | 2.22E−01 | 1.65E−01 | 1.18E−01 | 2.85E−02 | 8.44E−02 | 9.60E−02 |
| PROC | NM_000312 | 1.72E−01 | 1.83E−01 | 7.71E−02 | 1.47E−01 | 7.76E−02 | 5.72E−02 |
| COQ6 | NM_182480 | 3.81E−01 | 4.03E−02 | 1.58E−01 | 5.25E−03 | 1.14E−01 | 1.57E−02 |
| C12orf29 | NM_001009894 | 5.99E−03 | 1.85E−01 | 4.59E−03 | 3.25E−01 | 1.96E−02 | 1.74E−01 |
| CDC27 | NM_001114091 | 1.37E−01 | 7.52E−02 | 1.39E−01 | 7.15E−02 | 2.11E−01 | 8.09E−02 |
| PCDHGB1 | NM_018922 | 1.67E−01 | 9.02E−02 | 1.19E−01 | 9.76E−02 | 1.47E−01 | 9.43E−02 |
| SS18L1 | NM_198935 | 1.03E−01 | 7.24E−02 | 6.72E−02 | 1.89E−01 | 6.04E−02 | 2.27E−01 |
| KLK10 | NM_002776 | 8.38E−02 | 1.29E−01 | 8.89E−02 | 2.30E−01 | 4.47E−02 | 1.44E−01 |
| GJB3 | NM_001005752 | 2.85E−01 | 8.08E−03 | 6.98E−02 | 1.41E−01 | 4.54E−02 | 1.71E−01 |
| TXLNA | NM_175852 | 2.04E−01 | 2.15E−02 | 2.18E−01 | 1.61E−02 | 2.53E−01 | 8.78E−03 |
| YWHAZ | NM_001135699 | 7.49E−02 | 2.61E−01 | 1.84E−02 | 1.81E−01 | 2.04E−02 | 1.66E−01 |
| C10orf82 | NM_144661 | 2.17E−01 | 3.85E−02 | 1.58E−01 | 1.18E−01 | 3.70E−02 | 1.54E−01 |
| FOXI1 | NM_012188 | 2.99E−03 | 3.05E−01 | 1.83E−02 | 9.67E−02 | 9.48E−02 | 2.07E−01 |
| RTN2 | NM_206900 | 3.76E−01 | 1.25E−01 | 9.13E−02 | 5.30E−02 | 6.07E−02 | 2.00E−02 |
| LSP1 | NM_001289005 | 5.40E−03 | 2.27E−01 | 1.45E−03 | 2.33E−01 | 1.28E−01 | 1.32E−01 |
| PAAF1 | NM_001267803 | 2.01E−02 | 1.43E−02 | 3.93E−02 | 2.74E−01 | 9.32E−02 | 2.87E−01 |
| HAVCR1 | NM_001173393 | 6.13E−02 | 7.73E−02 | 1.91E−01 | 9.59E−02 | 1.90E−01 | 1.13E−01 |
| MGMT | NM_002412 | 2.47E−01 | 2.40E−03 | 1.86E−01 | 2.56E−02 | 2.16E−01 | 5.21E−02 |
| MBNL3 | NM_133486 | 5.50E−02 | 1.42E−01 | 1.46E−01 | 1.52E−01 | 9.86E−02 | 1.36E−01 |
| SPTBN4 | NM_025213 | 1.77E−02 | 4.02E−01 | 4.14E−02 | 1.70E−01 | 7.42E−02 | 2.69E−02 |
| AMER1 | NM_152424 | 8.45E−05 | 1.95E−01 | 2.42E−03 | 2.13E−01 | 1.09E−01 | 2.14E−01 |
| TMEM235 | NM_001204212 | 1.19E−01 | 1.59E−01 | 4.81E−02 | 2.02E−01 | 3.98E−02 | 1.65E−01 |
| IL20RB | NM_144717 | 9.20E−02 | 4.85E−02 | 2.31E−01 | 1.08E−02 | 3.23E−01 | 2.77E−02 |
| SULT2B1 | NM_177973 | 6.77E−02 | 4.09E−02 | 4.07E−01 | 6.11E−03 | 2.09E−01 | 3.02E−03 |
| SPAG8 | NM_172312 | 2.30E−02 | 1.72E−01 | 1.26E−02 | 2.24E−01 | 4.26E−03 | 2.98E−01 |
| FLYWCH2 | NM_001142499 | 1.21E−01 | 4.01E−02 | 5.71E−02 | 2.76E−01 | 8.82E−03 | 2.32E−01 |
| CPEB1 | NM_030594 | 5.19E−02 | 5.07E−02 | 1.46E−02 | 2.21E−01 | 1.10E−01 | 2.87E−01 |
| SYNE3 | NM_152592 | 4.09E−01 | 9.90E−02 | 1.12E−01 | 2.76E−02 | 3.96E−02 | 4.89E−02 |
| PTDSS2 | NM_030783 | 5.90E−02 | 2.15E−01 | 2.93E−02 | 1.79E−01 | 3.32E−02 | 2.21E−01 |
| NPB | NM_148896 | 2.12E−01 | 1.49E−01 | 1.34E−01 | 2.91E−02 | 1.39E−01 | 7.29E−02 |
| P2RX1 | NM_002558 | 8.81E−02 | 1.92E−01 | 6.84E−02 | 6.24E−02 | 2.16E−01 | 1.12E−01 |
| TESPA1 | NM_001136030 | 1.22E−01 | 1.33E−01 | 7.90E−02 | 8.87E−02 | 1.71E−01 | 1.46E−01 |
| SMO | NM_005631 | 1.78E−01 | 7.10E−04 | 1.66E−01 | 4.47E−02 | 3.15E−01 | 3.53E−02 |
| SMARCA2 | NM_001289396 | 9.67E−02 | 4.03E−01 | 1.41E−02 | 1.37E−01 | 2.65E−03 | 8.74E−02 |
| RAPGEF6 | NM_016340 | 3.70E−02 | 2.74E−03 | 1.41E−01 | 2.15E−02 | 5.18E−01 | 2.00E−02 |
| DDX47 | NM_016355 | 2.51E−01 | 2.76E−02 | 2.31E−01 | 7.40E−03 | 2.03E−01 | 2.17E−02 |
| TMEM52 | NM_178545 | 3.04E−01 | 1.20E−01 | 8.43E−02 | 5.79E−02 | 6.72E−02 | 1.09E−01 |
| POU4F1 | NM_006237 | 1.93E−01 | 1.70E−01 | 1.49E−01 | 3.43E−02 | 1.73E−01 | 2.32E−02 |
| LMX1B | NM_001174147 | 2.40E−01 | 3.09E−03 | 3.04E−01 | 1.16E−03 | 1.91E−01 | 3.92E−03 |
| NSUN3 | NM_022072 | 2.98E−02 | 5.35E−02 | 4.29E−02 | 2.26E−01 | 8.43E−02 | 3.07E−01 |
| ZNF135 | NM_001289401 | 1.89E−01 | 3.27E−01 | 4.94E−02 | 5.85E−02 | 6.53E−02 | 5.50E−02 |
| RNF24 | NM_001134338 | 9.63E−02 | 1.40E−03 | 1.45E−01 | 6.55E−02 | 3.55E−01 | 8.04E−02 |
| ZNF81 | NM_007137 | 3.00E−03 | 4.61E−01 | 3.35E−03 | 9.80E−02 | 1.16E−02 | 1.68E−01 |
| SHCBP1L | NM_030933 | 7.70E−02 | 9.54E−02 | 1.32E−01 | 9.51E−02 | 2.26E−01 | 1.19E−01 |
| DEFB127 | NM_139074 | 8.77E−02 | 3.14E−01 | 5.64E−02 | 1.02E−01 | 3.22E−02 | 1.53E−01 |
| TAF7L | NM_024885 | 6.33E−02 | 1.52E−01 | 7.41E−02 | 7.73E−02 | 3.25E−01 | 5.45E−02 |
| EIF4EBP1 | NM_004095 | 1.87E−01 | 4.34E−02 | 1.89E−01 | 7.00E−02 | 8.93E−02 | 1.68E−01 |
| YTHDF1 | NM_017798 | 1.75E−02 | 3.32E−01 | 1.10E−01 | 7.49E−02 | 1.16E−01 | 9.62E−02 |
| AWAT2 | NM_001002254 | 6.28E−02 | 1.04E−01 | 2.32E−01 | 2.08E−02 | 2.93E−01 | 3.42E−02 |
| KRT18 | NM_199187 | 2.08E−01 | 2.76E−01 | 1.19E−01 | 2.52E−02 | 9.78E−02 | 2.22E−02 |
| TACR2 | NM_001057 | 2.75E−02 | 1.37E−01 | 1.03E−01 | 1.50E−01 | 1.49E−01 | 1.82E−01 |
| NTRK3 | NM_002530 | 2.02E−01 | 2.99E−01 | 1.25E−01 | 2.29E−02 | 8.98E−02 | 1.15E−02 |
| KRT83 | NM_002282 | 2.14E−01 | 2.53E−01 | 1.22E−01 | 3.15E−02 | 8.92E−02 | 4.15E−02 |
| MAPK9 | NM_139070 | 5.17E−02 | 6.80E−02 | 1.27E−02 | 2.52E−01 | 1.27E−01 | 2.40E−01 |
| UBL4B | NM_203412 | 1.77E−01 | 1.29E−01 | 1.80E−01 | 3.23E−02 | 1.85E−01 | 4.77E−02 |
| PMM1 | NM_002676 | 2.05E−02 | 2.84E−02 | 8.20E−02 | 1.64E−01 | 3.29E−01 | 1.27E−01 |
| FAM124B | NM_024785 | 1.73E−01 | 5.84E−02 | 1.07E−01 | 1.05E−01 | 1.48E−01 | 1.61E−01 |
| PPAT | NM_002703 | 4.15E−01 | 3.40E−02 | 1.10E−01 | 2.16E−02 | 1.43E−01 | 2.89E−02 |
| DAZAP2 | NM_001136269 | 1.89E−01 | 3.21E−01 | 4.01E−02 | 8.32E−02 | 7.23E−02 | 4.67E−02 |
| TTLL3 | NM_001025930 | 1.69E−01 | 1.98E−01 | 6.23E−02 | 1.57E−01 | 1.31E−02 | 1.54E−01 |
| OR1N1 | NM_012363 | 1.19E−02 | 1.35E−01 | 1.20E−01 | 2.09E−01 | 2.06E−01 | 7.10E−02 |
| GATC | NM_176818 | 7.38E−02 | 2.95E−01 | 1.66E−01 | 9.20E−02 | 6.18E−02 | 6.47E−02 |
| DCP1A | NM_001290204 | 2.05E−03 | 2.96E−01 | 6.54E−03 | 2.04E−01 | 7.25E−03 | 2.39E−01 |
| ZMYM3 | NM_001171162 | 3.26E−01 | 1.61E−01 | 7.14E−02 | 5.64E−02 | 4.31E−02 | 9.63E−02 |
| FGF14 | NM_004115 | 2.33E−01 | 1.60E−02 | 2.13E−01 | 1.99E−03 | 2.85E−01 | 5.88E−03 |
| FBXO6 | NM_018438 | 1.87E−01 | 1.11E−01 | 1.47E−01 | 9.25E−02 | 1.02E−01 | 1.18E−01 |
| C8orf33 | NM_023080 | 3.40E−03 | 2.00E−01 | 6.78E−05 | 2.53E−01 | 2.90E−05 | 3.05E−01 |
| CTBP1 | NM_001012614 | 2.25E−01 | 1.60E−02 | 3.60E−01 | 6.68E−04 | 1.57E−01 | 3.12E−03 |
| ZDHHC8 | NM_013373 | 1.46E−01 | 2.47E−01 | 1.22E−01 | 9.91E−02 | 3.31E−02 | 1.15E−01 |
| DMXL1 | NM_005509 | 1.94E−01 | 9.10E−02 | 1.74E−01 | 4.84E−02 | 1.32E−01 | 1.23E−01 |
| TUSC3 | NM_006765 | 1.63E−01 | 9.84E−02 | 1.68E−01 | 1.90E−01 | 1.30E−01 | 1.30E−02 |
| SPATA31A5 | NM_001113541 | 4.35E−03 | 1.01E−02 | 1.45E−01 | 1.48E−01 | 3.57E−01 | 9.89E−02 |
| C17orf67 | NM_001085430 | 6.47E−02 | 2.12E−01 | 2.18E−01 | 5.30E−02 | 1.10E−01 | 1.06E−01 |
| MYL6B | NM_002475 | 2.04E−01 | 3.09E−02 | 1.38E−01 | 2.39E−01 | 7.25E−02 | 8.01E−02 |
| CAPN1 | NM_005186 | 1.28E−01 | 1.41E−02 | 2.18E−01 | 1.87E−01 | 1.96E−01 | 2.21E−02 |
| PGM5 | NM_021965 | 2.36E−01 | 2.67E−01 | 1.69E−01 | 1.82E−03 | 6.92E−02 | 2.42E−02 |
| HSD17B1 | NM_000413 | 1.43E−01 | 6.21E−02 | 3.25E−01 | 7.40E−02 | 6.78E−02 | 9.61E−02 |
| DSEL | NM_032160 | 5.13E−03 | 3.53E−01 | 5.49E−03 | 2.09E−01 | 1.01E−02 | 1.86E−01 |
| OSTF1 | NM_012383 | 1.36E−01 | 3.59E−02 | 7.06E−02 | 1.65E−01 | 1.33E−01 | 2.30E−01 |
| KDM2A | NM_001256405 | 8.49E−02 | 3.04E−01 | 2.90E−01 | 5.12E−02 | 3.45E−02 | 1.09E−02 |
| SP100 | NM_001206704 | 3.54E−01 | 4.40E−02 | 1.62E−01 | 3.99E−02 | 7.42E−02 | 1.03E−01 |
| DNAH5 | NM_001369 | 5.09E−02 | 2.60E−02 | 2.76E−01 | 6.36E−02 | 2.74E−01 | 8.75E−02 |
| GFPT2 | NM_005110 | 2.46E−02 | 4.24E−02 | 9.78E−02 | 2.36E−01 | 2.99E−01 | 7.81E−02 |
| EIF5A | NM_001143760 | 3.36E−01 | 1.09E−01 | 9.11E−02 | 1.81E−01 | 1.73E−02 | 4.37E−02 |
| REC8 | NM_001048205 | 3.01E−02 | 3.95E−01 | 1.81E−02 | 6.51E−02 | 2.12E−02 | 2.50E−01 |
| ZFAND6 | NM_001242917 | 1.13E−01 | 2.79E−01 | 8.42E−02 | 1.30E−01 | 4.75E−02 | 1.26E−01 |
| PACRG | NM_001080378 | 1.42E−02 | 2.51E−01 | 9.76E−02 | 1.46E−01 | 1.44E−01 | 1.28E−01 |
| TNFAIP2 | NM_006291 | 4.83E−02 | 2.31E−01 | 5.86E−02 | 1.06E−01 | 1.83E−01 | 1.55E−01 |
| KIAA1377 | NM_020802 | 1.18E−01 | 7.46E−02 | 2.29E−01 | 7.60E−02 | 2.43E−01 | 4.11E−02 |
| ZNF354A | NM_005649 | 1.44E−01 | 2.14E−01 | 1.91E−01 | 2.85E−02 | 1.54E−01 | 4.99E−02 |
| OPRL1 | NM_000913 | 1.82E−02 | 1.50E−01 | 1.11E−02 | 2.47E−01 | 3.71E−02 | 3.20E−01 |
| KLHL32 | NM_001286252 | 6.15E−02 | 1.08E−01 | 1.94E−01 | 6.93E−02 | 2.24E−01 | 1.27E−01 |
| MOSPD1 | NM_019556 | 7.55E−02 | 3.80E−02 | 3.21E−02 | 2.31E−01 | 1.64E−01 | 2.44E−01 |
| SARDH | NM_007101 | 7.40E−02 | 1.57E−01 | 1.21E−01 | 1.67E−01 | 8.48E−02 | 1.81E−01 |
| NPFF | NM_003717 | 8.05E−02 | 5.05E−02 | 1.86E−02 | 3.15E−01 | 1.02E−01 | 2.18E−01 |
| ATP8B3 | NM_138813 | 4.19E−02 | 4.81E−01 | 3.14E−02 | 7.02E−02 | 1.74E−02 | 1.44E−01 |
| PDIA6 | NM_001282705 | 2.20E−01 | 2.11E−01 | 5.28E−02 | 1.46E−01 | 2.75E−02 | 1.29E−01 |
| RRP1 | NM_003683 | 3.29E−01 | 7.85E−03 | 2.40E−01 | 4.30E−02 | 1.17E−01 | 5.04E−02 |
| ALG1 | NM_019109 | 9.22E−02 | 1.22E−01 | 1.32E−01 | 7.05E−02 | 2.94E−01 | 7.70E−02 |
| TMEM135 | NM_001168724 | 3.23E−02 | 4.18E−01 | 1.25E−02 | 1.05E−01 | 5.69E−02 | 1.67E−01 |
| OR2B2 | NM_033057 | 5.65E−01 | 8.25E−03 | 1.07E−01 | 2.69E−02 | 2.74E−02 | 5.72E−02 |
| MEFV | NM_001198536 | 3.73E−01 | 2.43E−01 | 1.49E−01 | 6.10E−03 | 1.51E−02 | 5.38E−03 |
| C6orf48 | NM_001287484 | 1.43E−01 | 3.65E−02 | 7.44E−02 | 8.42E−02 | 3.02E−01 | 1.52E−01 |
| KIAA0020 | NM_014878 | 1.81E−01 | 6.32E−02 | 1.34E−01 | 2.71E−01 | 8.78E−02 | 5.60E−02 |
| MCF2L | NM_001112732 | 4.01E−02 | 7.36E−03 | 1.03E−01 | 1.93E−01 | 1.58E−01 | 2.91E−01 |
| HIST1H2BK | NM_080593 | 7.42E−02 | 1.67E−01 | 5.51E−02 | 7.49E−02 | 3.09E−01 | 1.13E−01 |
| PRR5 | NM_001017528 | 1.61E−01 | 2.47E−01 | 1.64E−01 | 7.81E−02 | 1.04E−01 | 3.98E−02 |
| IRGC | NM_019612 | 3.19E−01 | 1.46E−01 | 1.67E−01 | 2.30E−02 | 8.43E−02 | 5.50E−02 |
| ACOT7 | NM_181864 | 6.73E−02 | 1.29E−01 | 1.58E−01 | 1.49E−01 | 7.53E−02 | 2.16E−01 |
| RPS15A | NM_001019 | 3.87E−01 | 5.00E−02 | 4.77E−02 | 8.60E−02 | 1.24E−02 | 2.12E−01 |
| GDA | NM_001242506 | 5.56E−03 | 4.29E−01 | 1.27E−02 | 6.82E−02 | 1.86E−01 | 9.64E−02 |
| PCDH1 | NM_001278613 | 1.01E−01 | 1.00E−01 | 1.46E−01 | 3.05E−02 | 3.89E−01 | 3.13E−02 |
| TNNT1 | NM_003283 | 4.64E−01 | 3.71E−02 | 7.21E−02 | 2.68E−02 | 1.96E−01 | 2.28E−03 |
| ACKR1 | NM_001122951 | 4.98E−02 | 2.06E−02 | 1.13E−01 | 3.03E−01 | 7.27E−02 | 2.40E−01 |
| CDC42SE2 | NM_001038702 | 8.72E−02 | 7.52E−02 | 1.76E−01 | 8.13E−02 | 1.72E−01 | 2.07E−01 |
| SLC9A7 | NM_032591 | 4.35E−02 | 4.52E−01 | 6.55E−02 | 7.77E−02 | 4.44E−02 | 1.17E−01 |
| C7orf26 | NM_024067 | 6.13E−02 | 2.33E−01 | 1.67E−01 | 1.56E−01 | 6.49E−02 | 1.19E−01 |
| MBD6 | NM_052897 | 1.25E−02 | 6.13E−01 | 2.55E−02 | 5.40E−02 | 5.78E−02 | 3.83E−02 |
| LYPLA1 | NM_001279356 | 1.10E−01 | 1.52E−01 | 2.07E−01 | 6.22E−02 | 2.23E−01 | 4.75E−02 |
| PELP1 | NM_014389 | 2.81E−01 | 2.03E−02 | 2.28E−01 | 4.33E−02 | 1.77E−01 | 5.18E−02 |
| SLC2A8 | NM_014580 | 2.31E−01 | 2.33E−01 | 1.95E−01 | 7.86E−03 | 1.29E−01 | 7.04E−03 |
| ODF3L2 | NM_182577 | 1.58E−01 | 2.00E−01 | 8.31E−04 | 2.48E−01 | 7.68E−03 | 1.88E−01 |
| IFNA10 | NM_002171 | 1.14E−01 | 3.20E−01 | 8.98E−02 | 8.77E−02 | 9.16E−02 | 1.01E−01 |
| LY6E | NM_002346 | 4.39E−01 | 1.14E−02 | 1.85E−01 | 4.67E−02 | 4.03E−02 | 8.12E−02 |
| CHMP2B | NM_014043 | 2.24E−02 | 3.77E−01 | 1.65E−02 | 1.27E−01 | 1.40E−01 | 1.19E−01 |
| NINJ2 | NM_016533 | 2.76E−01 | 1.25E−01 | 1.63E−01 | 3.84E−02 | 1.35E−01 | 6.71E−02 |
| TM7SF3 | NM_016551 | 2.07E−01 | 2.11E−01 | 9.42E−02 | 1.67E−02 | 2.41E−01 | 3.54E−02 |
| ATP6V0D2 | NM_152565 | 4.08E−02 | 3.79E−01 | 6.12E−02 | 9.44E−02 | 8.93E−02 | 1.39E−01 |
| C1QTNF3 | NM_181435 | 4.54E−03 | 1.83E−01 | 2.69E−02 | 3.09E−01 | 4.43E−02 | 2.38E−01 |
| SLC26A8 | NM_001193476 | 3.18E−01 | 4.65E−02 | 1.12E−01 | 1.51E−01 | 3.55E−02 | 1.43E−01 |
| SPRR1B | NM_003125 | 1.79E−01 | 6.23E−02 | 2.24E−01 | 8.70E−02 | 1.40E−01 | 1.15E−01 |
| WNT8A | NM_058244 | 2.49E−01 | 1.33E−01 | 2.39E−01 | 1.03E−02 | 1.64E−01 | 1.16E−02 |
| EDARADD | NM_080738 | 4.96E−02 | 1.07E−01 | 2.67E−01 | 6.86E−02 | 2.62E−01 | 5.44E−02 |
| GSDMB | NM_001042471 | 1.66E−01 | 2.98E−01 | 3.58E−02 | 7.79E−02 | 1.34E−01 | 9.66E−02 |
| KCNK2 | NM_001017424 | 1.43E−02 | 2.60E−01 | 1.37E−02 | 1.73E−01 | 1.86E−01 | 1.62E−01 |
| IQCA1 | NM_001270585 | 1.86E−02 | 3.76E−01 | 1.21E−02 | 1.07E−01 | 1.79E−01 | 1.19E−01 |
| NKX2-1 | NM_003317 | 8.42E−02 | 6.66E−02 | 3.26E−01 | 2.20E−02 | 2.81E−01 | 3.28E−02 |
| ZNF627 | NM_145295 | 5.29E−01 | 1.13E−02 | 1.09E−01 | 4.25E−02 | 3.36E−02 | 8.84E−02 |
| SLC1A5 | NM_001145145 | 1.13E−01 | 2.65E−01 | 8.71E−02 | 1.56E−01 | 1.44E−01 | 4.97E−02 |
| SETDB2 | NM_001160308 | 9.40E−03 | 2.84E−01 | 3.37E−02 | 2.81E−01 | 3.51E−02 | 1.73E−01 |
| LRRC49 | NM_017691 | 1.51E−02 | 1.03E−01 | 1.94E−02 | 2.21E−01 | 1.72E−01 | 2.86E−01 |
| RCN2 | NM_002902 | 1.01E−01 | 1.04E−01 | 6.42E−02 | 2.19E−01 | 1.39E−02 | 3.14E−01 |
| FBXO7 | NM_012179 | 1.04E−01 | 3.13E−01 | 2.51E−02 | 1.53E−01 | 6.47E−03 | 2.15E−01 |
| PACRG | NM_001080379 | 7.02E−02 | 1.02E−01 | 3.61E−02 | 2.89E−01 | 2.27E−02 | 2.97E−01 |
| SEPTIN9 | NM_001113496 | 1.32E−01 | 1.82E−02 | 4.08E−01 | 1.51E−02 | 2.08E−01 | 3.51E−02 |
| LMO3 | NM_001243610 | 1.30E−01 | 3.59E−02 | 1.47E−01 | 5.75E−02 | 3.35E−01 | 1.13E−01 |
| LDHA | NM_001165414 | 1.53E−01 | 1.20E−01 | 2.62E−01 | 3.60E−02 | 2.02E−01 | 4.57E−02 |
| MSRB1 | NM_016332 | 6.34E−02 | 3.34E−01 | 2.22E−01 | 6.51E−03 | 1.19E−01 | 7.64E−02 |
| RCOR3 | NM_001136223 | 1.53E−01 | 2.28E−01 | 1.89E−01 | 1.82E−02 | 1.87E−01 | 4.50E−02 |
| ZNF165 | NM_003447 | 1.06E−01 | 5.67E−02 | 2.11E−01 | 3.20E−02 | 3.54E−01 | 6.14E−02 |
| ASNSD1 | NM_019048 | 2.06E−01 | 2.11E−01 | 2.78E−01 | 1.09E−02 | 9.79E−02 | 1.80E−02 |
| ARHGAP26 | NM_015071 | 3.15E−01 | 1.06E−01 | 6.71E−02 | 1.86E−01 | 1.62E−02 | 1.33E−01 |
| ZNF443 | NM_005815 | 3.15E−01 | 1.73E−01 | 1.44E−01 | 2.66E−02 | 9.44E−02 | 7.09E−02 |
| STC1 | NM_003155 | 1.54E−01 | 6.08E−02 | 1.95E−01 | 2.74E−02 | 3.06E−01 | 8.18E−02 |
| ASB4 | NM_016116 | 1.87E−01 | 3.59E−02 | 2.36E−01 | 1.08E−01 | 2.01E−01 | 5.81E−02 |
| ZNF93 | NM_031218 | 1.76E−01 | 1.19E−01 | 1.58E−01 | 8.21E−02 | 1.55E−01 | 1.36E−01 |
| GSTZ1 | NM_145870 | 8.63E−02 | 1.86E−01 | 2.87E−01 | 2.12E−02 | 2.36E−01 | 9.08E−03 |
| AASDH | NM_181806 | 3.70E−02 | 1.87E−01 | 1.62E−01 | 8.94E−02 | 2.60E−01 | 9.19E−02 |
| CDK2AP2 | NM_001271849 | 5.14E−02 | 6.48E−02 | 3.47E−01 | 5.10E−03 | 3.52E−01 | 7.11E−03 |
| CDKN2B | NM_004936 | 9.45E−02 | 1.73E−01 | 1.64E−01 | 1.39E−01 | 2.05E−01 | 5.17E−02 |
| QARS | NM_005051 | 9.27E−04 | 4.77E−02 | 7.38E−02 | 2.39E−01 | 1.09E−01 | 3.57E−01 |
| NUP50 | NM_153645 | 1.35E−02 | 1.36E−01 | 8.99E−02 | 1.35E−01 | 2.17E−01 | 2.36E−01 |
| SCNN1G | NM_001039 | 8.01E−02 | 8.96E−02 | 2.92E−02 | 2.23E−01 | 1.74E−01 | 2.32E−01 |
| PRDX6 | NM_004905 | 3.35E−01 | 2.05E−03 | 1.95E−01 | 7.33E−03 | 2.77E−01 | 1.32E−02 |
| MLEC | NM_014730 | 3.61E−02 | 1.63E−01 | 4.52E−02 | 1.74E−01 | 1.00E−01 | 3.12E−01 |
| HMMR | NM_012484 | 4.55E−01 | 6.93E−03 | 2.34E−01 | 6.80E−03 | 1.26E−01 | 2.45E−03 |
| ENAH | NM_018212 | 1.34E−01 | 5.83E−02 | 2.70E−01 | 8.19E−02 | 2.63E−01 | 2.32E−02 |
| CNGB3 | NM_019098 | 2.46E−01 | 1.17E−01 | 8.16E−02 | 1.54E−01 | 4.79E−02 | 1.87E−01 |
| ADCK3 | NM_020247 | 1.12E−01 | 1.36E−01 | 2.76E−02 | 1.75E−02 | 4.51E−01 | 8.87E−02 |
| SH2D4B | NM_001145719 | 2.20E−02 | 1.32E−01 | 3.84E−02 | 1.84E−01 | 2.65E−01 | 1.92E−01 |
| EEF1A1 | NM_001402 | 1.87E−01 | 9.89E−02 | 1.95E−01 | 1.29E−01 | 2.11E−01 | 1.23E−02 |
| GPATCH3 | NM_022078 | 2.95E−01 | 1.00E−01 | 1.87E−01 | 8.01E−02 | 1.60E−01 | 1.23E−02 |
| CBLC | NM_012116 | 2.12E−01 | 1.09E−01 | 6.18E−03 | 3.00E−01 | 2.86E−02 | 1.80E−01 |
| ATG4B | NM_178326 | 3.21E−02 | 4.31E−01 | 3.08E−02 | 1.73E−01 | 3.28E−02 | 1.36E−01 |
| TACR1 | NM_001058 | 1.25E−01 | 2.05E−01 | 1.81E−01 | 1.59E−01 | 2.19E−02 | 1.44E−01 |
| IGFBP4 | NM_001552 | 1.11E−01 | 6.39E−02 | 4.95E−02 | 1.67E−01 | 1.78E−01 | 2.67E−01 |
| CYP19A1 | NM_031226 | 1.38E−01 | 1.78E−01 | 1.70E−01 | 2.30E−01 | 6.59E−02 | 5.73E−02 |
| LHX6 | NM_001242333 | 1.30E−01 | 3.07E−01 | 2.37E−02 | 2.15E−01 | 5.16E−03 | 1.58E−01 |
| GPC5 | NM_004466 | 3.99E−01 | 7.31E−02 | 1.79E−01 | 1.36E−02 | 1.44E−01 | 3.10E−02 |
| TPTE2 | NM_001271850 | 4.31E−03 | 3.84E−01 | 4.90E−03 | 1.79E−01 | 1.28E−02 | 2.56E−01 |
| NBEAL2 | NM_015175 | 7.45E−02 | 9.07E−02 | 8.19E−02 | 2.26E−01 | 5.51E−02 | 3.13E−01 |
| AGPAT4 | NM_020133 | 6.35E−02 | 1.67E−01 | 9.50E−02 | 1.60E−01 | 8.27E−02 | 2.72E−01 |
| SERINC1 | NM_020755 | 8.25E−03 | 2.60E−01 | 4.07E−02 | 2.59E−01 | 7.80E−02 | 1.96E−01 |
| ALPPL2 | NM_031313 | 1.79E−01 | 5.27E−02 | 2.64E−01 | 8.36E−02 | 1.46E−01 | 1.17E−01 |
| DUSP22 | NM_020185 | 2.19E−01 | 6.61E−02 | 5.14E−02 | 1.47E−01 | 9.90E−02 | 2.60E−01 |
| NIPA1 | NM_001142275 | 2.32E−01 | 2.54E−01 | 3.98E−02 | 1.06E−01 | 3.43E−02 | 1.78E−01 |
| POU2F3 | NM_014352 | 1.36E−01 | 1.07E−01 | 1.99E−01 | 6.14E−02 | 2.57E−01 | 8.41E−02 |
| HTR5A | NM_024012 | 1.37E−01 | 1.01E−02 | 2.53E−01 | 6.21E−02 | 2.29E−01 | 1.53E−01 |
| TXNL1 | NM_004786 | 2.63E−01 | 9.32E−03 | 2.77E−01 | 1.40E−02 | 2.63E−01 | 1.75E−02 |
| RAPGEF4 | NM_007023 | 1.81E−01 | 2.20E−01 | 1.66E−01 | 6.68E−02 | 1.99E−01 | 1.20E−02 |
| HACE1 | NM_020771 | 5.17E−02 | 5.25E−03 | 3.64E−01 | 2.14E−02 | 3.71E−01 | 3.13E−02 |
| MSRB3 | NM_001193460 | 5.76E−02 | 2.80E−01 | 1.40E−01 | 1.13E−01 | 1.49E−01 | 1.07E−01 |
| PWWP2B | NM_138499 | 1.08E−01 | 2.43E−01 | 5.49E−02 | 1.13E−01 | 5.33E−02 | 2.74E−01 |
| SAYSD1 | NM_018322 | 1.20E−01 | 5.17E−02 | 2.31E−01 | 7.42E−02 | 3.37E−01 | 3.18E−02 |
| GPRC5C | NM_022036 | 2.60E−01 | 1.15E−01 | 1.81E−01 | 7.25E−03 | 2.71E−01 | 1.31E−02 |
| SPEG | NM_001173476 | 1.95E−01 | 3.50E−01 | 1.43E−01 | 2.99E−03 | 1.54E−01 | 2.20E−03 |
| GSTM4 | NM_000850 | 1.67E−01 | 5.24E−02 | 2.20E−01 | 2.77E−02 | 3.18E−01 | 6.28E−02 |
| CNTN1 | NM_001256064 | 1.30E−01 | 6.72E−02 | 1.79E−01 | 1.43E−01 | 2.04E−01 | 1.27E−01 |
| CTNND2 | NM_001332 | 1.97E−01 | 2.28E−01 | 9.40E−02 | 1.67E−01 | 1.29E−01 | 3.47E−02 |
| TANGO2 | NM_001283199 | 2.36E−01 | 1.33E−01 | 1.95E−01 | 3.82E−02 | 2.39E−01 | 7.49E−03 |
| TOMM34 | NM_006809 | 2.89E−02 | 4.00E−01 | 1.13E−02 | 1.93E−01 | 1.08E−01 | 1.09E−01 |
| FBXL12 | NM_017703 | 2.45E−01 | 1.81E−01 | 2.55E−01 | 3.03E−02 | 5.69E−02 | 8.24E−02 |
| ZNF692 | NM_001136036 | 1.13E−01 | 1.49E−01 | 7.88E−02 | 1.54E−01 | 1.22E−01 | 2.36E−01 |
| SOCS1 | NM_003745 | 7.47E−02 | 8.82E−02 | 2.38E−01 | 9.95E−02 | 2.31E−01 | 1.22E−01 |
| EPHA3 | NM_182644 | 1.39E−02 | 1.44E−01 | 1.20E−01 | 2.12E−01 | 3.35E−02 | 3.29E−01 |
| CTDNEP1 | NM_001143775 | 8.39E−02 | 7.50E−02 | 1.09E−01 | 9.80E−02 | 2.99E−01 | 1.88E−01 |
| VDAC1 | NM_003374 | 2.07E−01 | 1.41E−01 | 2.28E−01 | 1.13E−02 | 2.40E−01 | 2.70E−02 |
| CLDN19 | NM_001185117 | 2.35E−01 | 1.24E−01 | 1.98E−01 | 3.95E−02 | 2.05E−01 | 5.29E−02 |
| VSIG10L | NM_001163922 | 1.19E−01 | 1.27E−02 | 2.15E−01 | 1.32E−01 | 1.58E−01 | 2.17E−01 |
| VAC14 | NM_018052 | 1.98E−01 | 7.82E−02 | 1.08E−01 | 2.18E−01 | 1.12E−01 | 1.40E−01 |
| LTV1 | NM_032860 | 1.22E−01 | 3.20E−02 | 3.74E−01 | 7.24E−02 | 1.97E−01 | 5.94E−02 |
| SHCBP1 | NM_024745 | 5.24E−02 | 7.06E−02 | 9.09E−02 | 1.92E−01 | 2.15E−01 | 2.37E−01 |
| GAPVD1 | NM_001282680 | 1.24E−01 | 8.29E−02 | 1.29E−01 | 5.25E−02 | 2.65E−01 | 2.04E−01 |
| FBXO5 | NM_012177 | 1.12E−01 | 7.51E−02 | 2.98E−01 | 9.13E−02 | 2.65E−01 | 1.78E−02 |
| RASGRP1 | NM_001128602 | 8.40E−02 | 2.15E−01 | 5.66E−02 | 2.50E−01 | 4.88E−02 | 2.04E−01 |
| NSD1 | NM_172349 | 4.93E−02 | 3.02E−01 | 3.42E−02 | 1.01E−01 | 2.72E−01 | 1.02E−01 |
| TAP1 | NM_000593 | 8.40E−02 | 7.65E−03 | 4.47E−02 | 2.37E−01 | 1.49E−01 | 3.39E−01 |
| ZNF454 | NM_182594 | 5.27E−02 | 6.16E−02 | 1.24E−01 | 8.61E−02 | 3.53E−01 | 1.85E−01 |
| PHF21A | NM_016621 | 3.65E−01 | 8.68E−02 | 3.77E−02 | 8.97E−02 | 2.43E−02 | 2.58E−01 |
| PPP1R10 | NM_002714 | 5.98E−02 | 2.47E−01 | 2.55E−02 | 2.44E−01 | 1.21E−01 | 1.65E−01 |
| LRP1 | NM_002332 | 6.82E−02 | 1.13E−01 | 1.49E−01 | 1.33E−01 | 2.49E−01 | 1.50E−01 |
| SMARCA5 | NM_003601 | 3.70E−01 | 1.42E−01 | 1.43E−01 | 2.62E−02 | 1.47E−01 | 3.64E−02 |
| EDC3 | NM_025083 | 9.89E−03 | 4.19E−01 | 1.36E−02 | 1.92E−01 | 1.51E−01 | 8.00E−02 |
| BTK | NM_000061 | 4.34E−02 | 4.81E−02 | 8.57E−02 | 3.10E−01 | 9.25E−02 | 2.86E−01 |
| PAM16 | NM_016069 | 1.67E−03 | 2.99E−01 | 9.04E−03 | 1.34E−01 | 1.42E−01 | 2.80E−01 |
| NBR1 | NM_001291571 | 2.52E−03 | 9.47E−02 | 5.09E−02 | 2.49E−01 | 8.70E−02 | 3.83E−01 |
| C9orf139 | NM_207511 | 4.14E−01 | 1.71E−01 | 9.26E−02 | 1.46E−01 | 2.05E−02 | 2.38E−02 |
| EPC1 | NM_001282391 | 2.29E−02 | 2.92E−01 | 8.69E−02 | 2.32E−01 | 1.56E−01 | 7.80E−02 |
| WDR54 | NM_032118 | 2.63E−02 | 1.22E−01 | 2.23E−02 | 2.24E−01 | 1.34E−01 | 3.40E−01 |
| ZNF248 | NM_001267606 | 1.82E−02 | 7.33E−02 | 1.30E−01 | 1.01E−01 | 4.87E−01 | 5.97E−02 |
| ETS2 | NM_005239 | 2.11E−01 | 7.38E−02 | 2.74E−01 | 9.61E−03 | 2.84E−01 | 1.67E−02 |
| MBD2 | NM_003927 | 2.84E−01 | 4.46E−01 | 5.64E−02 | 6.20E−02 | 1.21E−02 | 8.05E−03 |
| CD81 | NM_004356 | 1.64E−01 | 1.51E−01 | 1.15E−01 | 4.61E−02 | 3.47E−01 | 4.57E−02 |
| ADAMTSL2 | NM_014694 | 8.25E−02 | 1.97E−01 | 1.78E−01 | 1.45E−01 | 1.17E−01 | 1.52E−01 |
| RARG | NM_001243731 | 3.51E−02 | 2.72E−01 | 4.11E−02 | 2.87E−01 | 7.25E−02 | 1.63E−01 |
| NRG3 | NM_001165973 | 7.76E−03 | 3.90E−01 | 1.25E−01 | 1.01E−01 | 1.69E−01 | 7.67E−02 |
| ADH5 | NM_000671 | 5.01E−02 | 7.53E−02 | 1.04E−01 | 2.25E−01 | 1.69E−01 | 2.47E−01 |
| NALCN | NM_052867 | 1.00E−01 | 5.19E−01 | 2.08E−02 | 1.01E−01 | 1.23E−02 | 1.18E−01 |
| SOSTDC1 | NM_015464 | 1.80E−01 | 2.43E−02 | 2.64E−01 | 2.87E−02 | 2.93E−01 | 8.36E−02 |
| MPI | NM_002435 | 1.32E−01 | 1.55E−01 | 1.00E−01 | 1.58E−01 | 1.33E−01 | 1.96E−01 |
| C20orf96 | NM_153269 | 2.39E−01 | 2.14E−02 | 2.54E−01 | 6.21E−03 | 3.46E−01 | 6.20E−03 |
| KRT79 | NM_175834 | 4.05E−02 | 7.86E−02 | 2.74E−02 | 2.53E−01 | 1.02E−01 | 3.73E−01 |
| CYBB | NM_000397 | 7.27E−02 | 3.60E−01 | 1.27E−01 | 1.23E−01 | 6.50E−02 | 1.28E−01 |
| PLA2G7 | NM_005084 | 2.20E−01 | 1.60E−02 | 2.33E−01 | 5.68E−02 | 3.16E−01 | 3.43E−02 |
| USP2 | NM_001243759 | 1.30E−01 | 7.89E−02 | 2.06E−01 | 1.05E−01 | 2.02E−01 | 1.54E−01 |
| FBXO15 | NM_001142958 | 5.41E−01 | 1.48E−03 | 1.53E−01 | 1.17E−02 | 1.49E−01 | 2.02E−02 |
| ROPN1 | NM_017578 | 1.41E−02 | 3.47E−01 | 5.29E−03 | 2.22E−01 | 5.24E−02 | 2.36E−01 |
| GZMM | NM_001258351 | 1.77E−01 | 1.67E−01 | 9.81E−02 | 1.82E−01 | 8.79E−02 | 1.65E−01 |
| H3F3C | NM_001013699 | 3.09E−01 | 4.58E−02 | 1.15E−01 | 4.92E−02 | 2.52E−01 | 1.07E−01 |
| ELP2 | NM_001242875 | 6.28E−02 | 2.55E−01 | 1.48E−01 | 1.24E−01 | 1.42E−01 | 1.46E−01 |
| PIWIL2 | NM_001135721 | 2.15E−01 | 2.20E−01 | 1.57E−02 | 1.54E−01 | 9.67E−04 | 2.73E−01 |
| EMC4 | NM_001286420 | 2.92E−01 | 2.11E−01 | 1.55E−01 | 3.46E−02 | 1.43E−01 | 4.42E−02 |
| CKAP5 | NM_001008938 | 1.32E−01 | 2.15E−01 | 6.77E−02 | 7.09E−02 | 2.95E−01 | 9.93E−02 |
| GSC | NM_173849 | 3.90E−02 | 1.21E−01 | 8.01E−02 | 2.81E−01 | 1.46E−01 | 2.12E−01 |
| AES | NM_198969 | 1.48E−01 | 2.31E−01 | 1.01E−01 | 8.28E−02 | 2.31E−01 | 8.53E−02 |
| SLCO5A1 | NM_001146008 | 2.01E−02 | 1.33E−02 | 1.09E−01 | 2.80E−01 | 3.10E−01 | 1.49E−01 |
| ARSH | NM_001011719 | 2.69E−02 | 2.38E−01 | 5.60E−02 | 1.27E−01 | 3.01E−01 | 1.33E−01 |
| WNK4 | NM_032387 | 3.08E−02 | 3.67E−01 | 1.20E−01 | 1.36E−01 | 6.49E−02 | 1.62E−01 |
| CPNE4 | NM_153429 | 2.30E−01 | 1.48E−02 | 3.26E−01 | 7.09E−02 | 1.80E−01 | 5.94E−02 |
| KIF25 | NM_030615 | 2.03E−01 | 2.94E−01 | 1.16E−01 | 9.14E−02 | 1.08E−01 | 7.11E−02 |
| LRIT1 | NM_015613 | 3.27E−01 | 1.08E−01 | 1.24E−01 | 1.21E−01 | 1.04E−01 | 9.85E−02 |
| CMTM1 | NM_181296 | 2.07E−01 | 2.52E−02 | 2.65E−01 | 1.48E−02 | 3.40E−01 | 3.08E−02 |
| ZNF687 | NM_020832 | 4.28E−01 | 2.77E−02 | 1.10E−01 | 5.45E−02 | 1.70E−01 | 9.37E−02 |
| TSSK3 | NM_052841 | 5.57E−02 | 3.70E−03 | 3.67E−01 | 3.54E−02 | 3.06E−01 | 1.17E−01 |
| CCDC155 | NM_144688 | 5.41E−02 | 5.01E−01 | 5.19E−02 | 1.22E−01 | 4.24E−02 | 1.13E−01 |
| KCNJ15 | NM_170736 | 1.13E−01 | 1.98E−01 | 5.75E−02 | 2.33E−01 | 8.70E−02 | 1.96E−01 |
| C2CD3 | NM_001286577 | 2.21E−01 | 1.84E−01 | 1.61E−01 | 5.49E−02 | 1.89E−01 | 7.58E−02 |
| KREMEN1 | NM_032045 | 3.27E−01 | 2.11E−01 | 1.64E−01 | 4.73E−02 | 1.00E−01 | 3.66E−02 |
| FOXP3 | NM_014009 | 1.41E−01 | 3.23E−02 | 1.02E−01 | 1.98E−01 | 6.39E−02 | 3.48E−01 |
| VASH2 | NM_001136474 | 3.49E−01 | 2.99E−02 | 2.41E−01 | 1.79E−02 | 2.22E−01 | 2.54E−02 |
| UBR5 | NM_015902 | 3.63E−01 | 2.71E−02 | 7.43E−02 | 1.96E−01 | 1.74E−01 | 5.13E−02 |
| OR2T33 | NM_001004695 | 6.11E−02 | 2.60E−01 | 5.14E−02 | 2.69E−01 | 1.95E−01 | 4.97E−02 |
| DZIP1L | NM_173543 | 4.99E−01 | 7.58E−03 | 1.37E−01 | 7.26E−02 | 5.78E−02 | 1.12E−01 |
| SMIM11 | NM_058182 | 2.95E−01 | 2.37E−01 | 5.70E−02 | 5.77E−02 | 1.68E−01 | 7.25E−02 |
| EBF1 | NM_024007 | 1.74E−01 | 2.53E−01 | 9.39E−02 | 1.11E−01 | 2.99E−02 | 2.25E−01 |
| HABP4 | NM_014282 | 3.74E−01 | 1.23E−01 | 1.74E−01 | 4.53E−02 | 1.07E−01 | 6.54E−02 |
| WDR61 | NM_025234 | 5.12E−01 | 4.50E−02 | 1.98E−01 | 9.73E−03 | 1.08E−01 | 1.64E−02 |
| VGLL3 | NM_016206 | 1.98E−01 | 7.02E−02 | 1.79E−01 | 1.68E−01 | 2.27E−01 | 4.80E−02 |
| CHST8 | NM_001127896 | 4.89E−01 | 6.58E−03 | 2.03E−01 | 3.72E−02 | 9.60E−02 | 5.93E−02 |
| CRISP3 | NM_001190986 | 1.41E−01 | 2.13E−01 | 2.07E−02 | 2.48E−01 | 3.70E−02 | 2.32E−01 |
| PPP1CA | NM_206873 | 3.50E−01 | 1.12E−01 | 1.70E−01 | 1.87E−02 | 2.10E−01 | 3.20E−02 |
| PIGZ | NM_025163 | 4.06E−01 | 8.95E−03 | 2.72E−01 | 5.57E−03 | 1.95E−01 | 6.80E−03 |
| TMEM255A | NM_001104544 | 2.06E−01 | 6.49E−03 | 2.16E−01 | 1.26E−01 | 3.12E−01 | 2.88E−02 |
| SPON1 | NM_006108 | 2.65E−01 | 1.40E−02 | 2.32E−01 | 1.01E−02 | 3.30E−01 | 4.39E−02 |
| SMARCB1 | NM_003073 | 4.62E−03 | 4.06E−01 | 3.37E−02 | 2.03E−01 | 8.90E−02 | 1.59E−01 |
| PTBP1 | NM_031991 | 7.17E−03 | 4.44E−01 | 1.26E−01 | 1.17E−01 | 5.54E−02 | 1.48E−01 |
| PPP1R27 | NM_001007533 | 3.54E−01 | 1.11E−01 | 1.68E−01 | 2.26E−02 | 9.10E−02 | 1.50E−01 |
| ATP2C1 | NM_001199181 | 1.89E−01 | 5.57E−02 | 1.41E−01 | 3.35E−01 | 1.09E−01 | 6.71E−02 |
| ADAM19 | NM_033274 | 5.49E−03 | 2.59E−01 | 5.85E−02 | 1.62E−01 | 9.24E−02 | 3.20E−01 |
| GKAP1 | NM_001135953 | 2.95E−01 | 9.24E−02 | 2.05E−01 | 5.54E−02 | 2.02E−01 | 4.70E−02 |
| NREP | NM_001142482 | 2.04E−01 | 1.46E−01 | 2.96E−02 | 2.23E−01 | 2.04E−02 | 2.74E−01 |
| SLC39A9 | NM_001252152 | 1.03E−01 | 1.77E−01 | 2.66E−01 | 1.31E−01 | 8.97E−02 | 1.31E−01 |
| NPC2 | NM_006432 | 2.94E−01 | 8.17E−02 | 1.83E−01 | 5.71E−02 | 1.90E−01 | 9.26E−02 |
| SYNJ1 | NM_003895 | 3.28E−01 | 5.69E−02 | 2.49E−01 | 7.52E−02 | 1.13E−01 | 7.61E−02 |
| ATP6V1G1 | NM_004888 | 4.18E−01 | 1.43E−02 | 1.77E−01 | 8.51E−02 | 1.61E−01 | 4.29E−02 |
| PITPNB | NM_001284278 | 5.89E−01 | 2.06E−02 | 2.36E−01 | 4.14E−03 | 4.39E−02 | 5.12E−03 |
| ZNF623 | NM_001261843 | 7.61E−02 | 1.91E−01 | 1.73E−01 | 1.83E−01 | 1.41E−01 | 1.35E−01 |
| DDX1 | NM_004939 | 8.09E−02 | 1.06E−01 | 1.08E−01 | 1.04E−01 | 4.02E−01 | 9.99E−02 |
| NUDT7 | NM_001243661 | 3.68E−02 | 6.96E−01 | 5.35E−02 | 2.37E−02 | 3.59E−02 | 5.50E−02 |
| S100A13 | NM_005979 | 1.56E−01 | 1.24E−01 | 7.52E−02 | 2.02E−01 | 5.21E−02 | 2.91E−01 |
| SEMA3C | NM_006379 | 2.81E−01 | 1.02E−01 | 1.90E−01 | 2.48E−01 | 2.68E−02 | 5.37E−02 |
| EOMES | NM_005442 | 3.15E−02 | 6.20E−02 | 1.12E−01 | 1.49E−01 | 4.55E−01 | 9.20E−02 |
| FBLN1 | NM_006487 | 2.53E−02 | 2.50E−01 | 1.45E−01 | 1.18E−01 | 1.97E−01 | 1.67E−01 |
| FAM131B | NM_014690 | 8.98E−02 | 3.16E−01 | 1.49E−01 | 9.61E−02 | 1.27E−01 | 1.24E−01 |
| MTARC2 | NM_017898 | 3.23E−03 | 1.16E−01 | 3.09E−02 | 3.27E−01 | 7.30E−02 | 3.53E−01 |
| TIFA | NM_052864 | 2.22E−01 | 4.73E−01 | 8.84E−03 | 9.42E−02 | 2.98E−03 | 1.03E−01 |
| RNF135 | NM_197939 | 1.82E−01 | 1.54E−01 | 2.22E−01 | 8.67E−02 | 1.65E−01 | 9.42E−02 |
| ALDH3B2 | NM_001031615 | 4.89E−02 | 5.20E−01 | 1.46E−02 | 6.81E−02 | 1.30E−01 | 1.24E−01 |
| COL8A1 | NM_001850 | 3.82E−02 | 3.54E−01 | 1.93E−02 | 2.83E−01 | 1.14E−03 | 2.11E−01 |
| CCT4 | NM_001256721 | 2.04E−01 | 2.79E−01 | 1.59E−01 | 1.50E−02 | 1.70E−01 | 7.96E−02 |
| QRICH1 | NM_017730 | 1.79E−01 | 2.00E−01 | 1.13E−01 | 1.63E−01 | 1.09E−01 | 1.43E−01 |
| RIOK2 | NM_018343 | 3.74E−01 | 2.08E−01 | 7.12E−02 | 1.18E−01 | 5.92E−02 | 7.72E−02 |
| EFHC1 | NM_018100 | 5.94E−02 | 5.65E−01 | 1.84E−03 | 1.40E−01 | 1.70E−02 | 1.24E−01 |
| AATK | NM_001080395 | 1.16E−01 | 3.64E−01 | 1.06E−01 | 1.89E−01 | 6.16E−02 | 7.06E−02 |
| FAM217B | NM_001190826 | 2.35E−01 | 1.77E−01 | 6.12E−02 | 1.97E−01 | 5.06E−02 | 1.86E−01 |
| CDK5RAP1 | NM_016082 | 2.68E−01 | 1.24E−01 | 1.19E−01 | 1.01E−01 | 2.60E−01 | 3.84E−02 |
| ITIH1 | NM_001166434 | 2.50E−01 | 1.52E−01 | 2.59E−02 | 1.81E−01 | 1.69E−01 | 1.33E−01 |
| FBXO34 | NM_152231 | 1.60E−01 | 2.55E−01 | 1.62E−01 | 3.33E−02 | 2.17E−01 | 8.38E−02 |
| ZFAT | NM_001289394 | 4.19E−01 | 5.28E−03 | 1.27E−01 | 1.73E−01 | 3.94E−02 | 1.47E−01 |
| RABGAP1L | NM_001243764 | 5.43E−01 | 2.09E−02 | 1.72E−01 | 3.69E−03 | 1.59E−01 | 1.18E−02 |
| KIF21B | NM_001252100 | 1.28E−01 | 1.47E−01 | 8.34E−02 | 1.50E−01 | 4.74E−02 | 3.57E−01 |
| GABARAP | NM_007278 | 1.39E−01 | 8.44E−04 | 3.60E−01 | 1.47E−01 | 2.61E−01 | 4.99E−03 |
| CLCN3 | NM_001243374 | 5.27E−02 | 3.99E−01 | 1.46E−02 | 1.42E−01 | 2.38E−01 | 6.67E−02 |
| RAD21 | NM_006265 | 2.78E−01 | 7.78E−02 | 2.63E−01 | 1.47E−02 | 2.54E−01 | 2.67E−02 |
| RNF165 | NM_152470 | 6.96E−02 | 2.46E−01 | 1.18E−01 | 1.93E−01 | 6.26E−02 | 2.25E−01 |
| EFCAB2 | NM_032328 | 3.67E−01 | 1.32E−01 | 4.78E−02 | 1.68E−01 | 4.93E−02 | 1.51E−01 |
| POTEH | NM_001136213 | 9.86E−02 | 3.14E−01 | 4.11E−02 | 1.35E−01 | 1.66E−01 | 1.61E−01 |
| TRIM73 | NM_198924 | 2.03E−01 | 1.67E−01 | 8.85E−02 | 1.13E−01 | 2.03E−01 | 1.42E−01 |
| LHFP | NM_005780 | 2.14E−01 | 3.84E−02 | 3.81E−01 | 1.96E−02 | 2.14E−01 | 5.01E−02 |
| PODN | NM_001199081 | 7.89E−02 | 3.59E−01 | 2.97E−02 | 2.43E−01 | 5.66E−03 | 2.01E−01 |
| SYT15 | NM_181519 | 1.16E−01 | 6.81E−02 | 2.55E−01 | 1.65E−01 | 8.68E−02 | 2.27E−01 |
| TNPO2 | NM_001136195 | 3.83E−01 | 4.18E−03 | 3.57E−01 | 6.78E−05 | 1.73E−01 | 6.78E−05 |
| KRT19 | NM_002276 | 6.80E−02 | 1.82E−01 | 1.12E−01 | 4.50E−02 | 4.93E−01 | 1.98E−02 |
| FAM178B | NM_001172667 | 1.35E−01 | 1.04E−02 | 2.81E−01 | 9.92E−02 | 2.82E−01 | 1.13E−01 |
| TBX1 | NM_080646 | 3.30E−01 | 3.73E−02 | 2.51E−01 | 3.02E−02 | 2.53E−01 | 1.87E−02 |
| MSL2 | NM_001145417 | 5.74E−02 | 2.09E−01 | 2.46E−01 | 3.85E−02 | 2.05E−01 | 1.65E−01 |
| ZNF30 | NM_194325 | 4.96E−01 | 8.12E−02 | 1.58E−01 | 5.43E−03 | 1.78E−01 | 3.18E−03 |
| C1orf158 | NM_152290 | 2.32E−01 | 2.10E−01 | 2.42E−01 | 1.72E−02 | 1.81E−01 | 3.93E−02 |
| HSPB7 | NM_014424 | 1.35E−01 | 1.94E−01 | 1.41E−01 | 3.81E−02 | 3.89E−01 | 2.50E−02 |
| MAG | NM_001199216 | 3.59E−01 | 2.59E−03 | 2.31E−01 | 1.43E−01 | 1.50E−01 | 3.76E−02 |
| LMO2 | NM_001142316 | 3.09E−01 | 2.37E−01 | 1.01E−01 | 9.22E−02 | 9.74E−02 | 8.73E−02 |
| PHYHIP | NM_001099335 | 1.11E−01 | 1.07E−01 | 2.84E−01 | 7.12E−02 | 2.35E−01 | 1.15E−01 |
| TIMM23B | NM_001290117 | 5.16E−02 | 9.58E−02 | 8.26E−02 | 2.57E−01 | 1.74E−01 | 2.63E−01 |
| PSMA5 | NM_001199773 | 1.45E−02 | 4.35E−01 | 2.15E−02 | 3.63E−01 | 3.17E−02 | 5.83E−02 |
| ATP11A | NM_032189 | 1.47E−01 | 1.05E−01 | 2.26E−01 | 3.07E−02 | 3.99E−01 | 1.74E−02 |
| C20orf78 | NM_001242671 | 2.16E−03 | 3.80E−01 | 3.38E−03 | 4.52E−02 | 1.07E−01 | 3.88E−01 |
| C20orf196 | NM_152504 | 4.20E−01 | 2.46E−02 | 1.46E−01 | 1.22E−01 | 7.57E−02 | 1.37E−01 |
| UHRF1BP1L | NM_015054 | 1.24E−01 | 2.35E−01 | 1.54E−02 | 3.25E−01 | 6.62E−03 | 2.19E−01 |
| PTCH1 | NM_000264 | 8.92E−02 | 6.71E−02 | 9.68E−02 | 2.54E−01 | 3.70E−01 | 5.00E−02 |
| RGS12 | NM_198227 | 2.31E−01 | 1.66E−01 | 1.36E−01 | 1.67E−01 | 6.90E−02 | 1.57E−01 |
| RCN3 | NM_020650 | 2.16E−01 | 1.18E−01 | 1.73E−01 | 9.84E−02 | 1.98E−01 | 1.24E−01 |
| NAT8L | NM_178557 | 4.77E−01 | 6.75E−03 | 2.17E−01 | 1.08E−03 | 2.26E−01 | 6.87E−04 |
| CCNI | NM_006835 | 7.90E−02 | 1.71E−01 | 1.22E−01 | 2.61E−01 | 1.59E−01 | 1.35E−01 |
| ECSCR | NM_001077693 | 1.16E−02 | 1.04E−01 | 9.27E−02 | 7.47E−02 | 5.49E−01 | 9.65E−02 |
| MTX3 | NM_001167741 | 3.91E−02 | 1.22E−01 | 1.37E−01 | 1.99E−01 | 2.93E−01 | 1.38E−01 |
| TENM3 | NM_001080477 | 2.03E−02 | 2.37E−01 | 5.10E−02 | 1.21E−01 | 2.46E−01 | 2.54E−01 |
| CTXN3 | NM_001048252 | 6.56E−02 | 6.34E−02 | 2.07E−01 | 1.35E−01 | 4.17E−01 | 4.08E−02 |
| ANAPC1 | NM_022662 | 1.86E−01 | 2.82E−01 | 1.49E−01 | 1.57E−01 | 1.07E−01 | 4.90E−02 |
| ZNF469 | NM_001127464 | 1.73E−02 | 5.91E−02 | 1.27E−01 | 2.62E−01 | 2.73E−01 | 1.91E−01 |
| FAM170A | NM_182761 | 4.13E−01 | 4.63E−02 | 1.99E−01 | 1.64E−01 | 9.10E−02 | 1.61E−02 |
| MBD3L2 | NM_144614 | 3.30E−02 | 5.76E−02 | 2.33E−01 | 7.55E−02 | 3.11E−01 | 2.21E−01 |
| PUSL1 | NM_153339 | 1.91E−01 | 4.31E−03 | 2.43E−01 | 7.21E−02 | 2.22E−01 | 1.99E−01 |
| DIS3L | NM_001143688 | 3.56E−01 | 2.28E−03 | 2.44E−01 | 6.34E−02 | 1.74E−01 | 9.17E−02 |
| AHCYL2 | NM_001130722 | 3.96E−01 | 1.92E−02 | 2.18E−01 | 1.68E−01 | 1.00E−01 | 3.12E−02 |
| HOXB4 | NM_024015 | 6.36E−01 | 5.45E−02 | 1.65E−01 | 1.13E−02 | 2.25E−02 | 4.32E−02 |
| YBEY | NM_058181 | 1.59E−02 | 1.67E−01 | 3.01E−02 | 3.41E−01 | 4.13E−02 | 3.38E−01 |
| DPH5 | NM_015958 | 4.60E−01 | 5.74E−02 | 1.73E−01 | 3.92E−02 | 1.11E−01 | 9.25E−02 |
| RREB1 | NM_001003699 | 1.24E−01 | 2.02E−02 | 3.00E−01 | 1.78E−01 | 3.06E−01 | 4.82E−03 |
| CNTNAP4 | NM_033401 | 2.07E−01 | 2.88E−02 | 2.98E−01 | 3.13E−02 | 3.64E−01 | 4.17E−03 |
| SIRT3 | NM_012239 | 5.75E−01 | 4.46E−03 | 2.83E−02 | 3.95E−02 | 1.37E−01 | 1.49E−01 |
| RHOB | NM_004040 | 5.35E−01 | 8.98E−02 | 1.70E−01 | 3.50E−02 | 4.00E−02 | 6.46E−02 |
| ZSWIM7 | NM_001042698 | 1.67E−01 | 4.11E−02 | 2.45E−01 | 1.53E−01 | 2.82E−01 | 4.69E−02 |
| CABP5 | NM_019855 | 5.31E−01 | 7.16E−02 | 1.57E−01 | 3.14E−02 | 3.67E−02 | 1.07E−01 |
| PPCS | NM_001287506 | 2.44E−01 | 2.31E−02 | 1.48E−01 | 1.60E−01 | 2.08E−01 | 1.54E−01 |
| MAMSTR | NM_001130915 | 9.08E−02 | 3.15E−01 | 7.19E−02 | 1.85E−01 | 5.99E−02 | 2.15E−01 |
| HEY1 | NM_001282851 | 1.83E−01 | 6.44E−02 | 9.20E−02 | 9.94E−02 | 3.92E−01 | 1.07E−01 |
| UBE2L3 | NM_001256355 | 6.50E−02 | 1.95E−02 | 3.79E−02 | 2.44E−01 | 2.93E−01 | 2.79E−01 |
| PPP1R37 | NM_019121 | 1.29E−01 | 4.51E−01 | 6.21E−02 | 1.11E−01 | 4.86E−02 | 1.37E−01 |
| CD3G | NM_000073 | 7.98E−02 | 2.77E−01 | 9.59E−03 | 1.94E−01 | 1.41E−03 | 3.79E−01 |
| GAR1 | NM_032993 | 2.33E−01 | 3.41E−01 | 1.48E−01 | 6.79E−02 | 8.18E−02 | 6.85E−02 |
| ZC3H15 | NM_018471 | 1.74E−02 | 4.51E−02 | 2.42E−02 | 3.34E−01 | 2.18E−02 | 4.98E−01 |
| F13B | NM_001994 | 1.82E−01 | 5.14E−02 | 1.17E−01 | 2.08E−01 | 1.48E−01 | 2.35E−01 |
| CACFD1 | NM_017586 | 2.24E−01 | 2.05E−01 | 2.58E−02 | 2.79E−01 | 8.67E−03 | 1.99E−01 |
| COX6B2 | NM_144613 | 1.23E−01 | 6.88E−03 | 3.57E−01 | 1.77E−02 | 4.09E−01 | 2.76E−02 |
| SPEN | NM_015001 | 1.26E−02 | 1.07E−01 | 4.07E−02 | 3.24E−01 | 9.16E−02 | 3.67E−01 |
| PHLDA3 | NM_012396 | 1.53E−02 | 5.65E−01 | 4.37E−02 | 1.13E−01 | 8.51E−02 | 1.20E−01 |
| IL1RL2 | NM_003854 | 1.16E−01 | 4.70E−02 | 9.57E−02 | 2.88E−01 | 2.21E−01 | 1.76E−01 |
| TMEM170A | NM_145254 | 2.82E−01 | 9.87E−02 | 1.60E−01 | 1.49E−01 | 9.11E−02 | 1.63E−01 |
| PBX3 | NM_006195 | 3.56E−02 | 4.76E−01 | 2.32E−04 | 1.32E−01 | 8.33E−05 | 3.00E−01 |
| ZNF718 | NM_001039127 | 1.51E−01 | 3.13E−02 | 8.09E−02 | 2.60E−01 | 1.79E−01 | 2.41E−01 |
| LYPD5 | NM_001288763 | 3.52E−01 | 2.39E−01 | 1.83E−01 | 3.76E−02 | 7.68E−02 | 5.58E−02 |
| TMEM51 | NM_001136218 | 2.81E−01 | 1.90E−01 | 5.60E−02 | 1.36E−01 | 5.63E−02 | 2.25E−01 |
| NFIA | NM_001134673 | 2.32E−01 | 1.92E−01 | 5.89E−02 | 1.55E−01 | 1.52E−01 | 1.55E−01 |
| ZNF711 | NM_021998 | 3.69E−02 | 6.18E−01 | 8.73E−03 | 2.32E−01 | 1.56E−02 | 3.37E−02 |
| UBE2E1 | NM_001202476 | 2.71E−01 | 2.66E−02 | 1.35E−01 | 1.79E−01 | 2.98E−01 | 3.72E−02 |
| VEGFA | NM_001171630 | 2.08E−02 | 1.55E−01 | 1.63E−01 | 3.02E−01 | 9.27E−02 | 2.13E−01 |
| GSTP1 | NM_000852 | 2.50E−01 | 1.24E−01 | 1.96E−01 | 9.11E−02 | 2.75E−01 | 1.08E−02 |
| KIAA1107 | NM_015237 | 1.61E−01 | 1.02E−01 | 1.61E−01 | 1.06E−01 | 2.23E−01 | 1.93E−01 |
| MXRA8 | NM_032348 | 7.89E−02 | 8.08E−02 | 1.96E−02 | 4.19E−01 | 1.36E−02 | 3.36E−01 |
| OSBPL1A | NM_080597 | 2.52E−01 | 1.75E−01 | 1.97E−01 | 1.28E−01 | 8.63E−02 | 1.11E−01 |
| VNN1 | NM_004666 | 2.83E−01 | 2.82E−02 | 1.53E−01 | 1.57E−01 | 9.46E−02 | 2.34E−01 |
| DEFB125 | NM_153325 | 3.21E−01 | 5.96E−02 | 2.49E−01 | 4.76E−02 | 1.95E−01 | 7.75E−02 |
| ZNF185 | NM_001178113 | 3.62E−01 | 1.96E−01 | 2.75E−01 | 4.32E−02 | 6.71E−02 | 6.71E−03 |
| GNGT2 | NM_001198754 | 7.74E−03 | 1.84E−01 | 8.78E−02 | 2.44E−01 | 1.40E−01 | 2.88E−01 |
| COG4 | NM_015386 | 2.61E−01 | 1.05E−01 | 1.29E−01 | 9.30E−02 | 2.53E−01 | 1.10E−01 |
| DIO3 | NM_001362 | 2.18E−01 | 7.79E−02 | 1.68E−01 | 5.38E−03 | 2.57E−01 | 2.26E−01 |
| SLC7A6 | NM_001076785 | 9.12E−02 | 3.97E−03 | 2.87E−01 | 2.12E−01 | 1.76E−01 | 1.82E−01 |
| GRIN3A | NM_133445 | 2.20E−01 | 8.68E−02 | 1.52E−01 | 2.01E−01 | 1.14E−01 | 1.78E−01 |
| SGCB | NM_000232 | 7.94E−03 | 3.03E−01 | 1.99E−02 | 2.75E−01 | 5.34E−03 | 3.41E−01 |
| TAF6 | NM_139315 | 5.50E−01 | 1.49E−02 | 1.74E−01 | 4.08E−02 | 1.19E−01 | 5.56E−02 |
| XRCC4 | NM_022550 | 3.69E−01 | 2.33E−01 | 4.28E−02 | 1.32E−01 | 6.46E−02 | 1.13E−01 |
| NAT8 | NM_003960 | 2.27E−02 | 4.06E−02 | 3.53E−02 | 4.21E−01 | 2.90E−02 | 4.05E−01 |
| HOXB13 | NM_006361 | 2.37E−01 | 1.73E−01 | 1.72E−01 | 1.34E−01 | 1.20E−01 | 1.18E−01 |
| FAM96B | NM_016062 | 3.31E−01 | 8.86E−03 | 2.65E−01 | 6.40E−02 | 1.46E−01 | 1.40E−01 |
| SLC16A3 | NM_004207 | 1.79E−01 | 1.21E−01 | 2.23E−01 | 9.14E−02 | 2.41E−01 | 1.01E−01 |
| NDUFA7 | NM_005001 | 1.94E−01 | 9.65E−02 | 2.44E−01 | 1.21E−02 | 3.90E−01 | 1.92E−02 |
| MYH4 | NM_017533 | 5.05E−01 | 1.18E−01 | 1.75E−01 | 8.39E−02 | 5.15E−02 | 2.27E−02 |
| FOXD4 | NM_207305 | 2.24E−02 | 3.01E−01 | 4.98E−02 | 3.19E−01 | 6.03E−02 | 2.04E−01 |
| PABPN1 | NM_004643 | 1.11E−01 | 4.69E−02 | 5.17E−02 | 3.41E−01 | 1.68E−01 | 2.38E−01 |
| TPCN1 | NM_017901 | 1.40E−01 | 4.05E−01 | 5.13E−02 | 2.28E−01 | 5.16E−02 | 8.08E−02 |
| PIANP | NM_001244015 | 4.07E−02 | 1.67E−01 | 7.10E−03 | 2.92E−01 | 3.87E−03 | 4.46E−01 |
| LTBP2 | NM_000428 | 1.06E−01 | 4.25E−01 | 3.74E−02 | 1.26E−01 | 3.94E−02 | 2.24E−01 |
| C17orf49 | NM_001142798 | 3.70E−01 | 1.19E−02 | 2.45E−01 | 4.82E−02 | 1.84E−01 | 1.01E−01 |
| SFT2D3 | NM_032740 | 1.95E−01 | 9.26E−02 | 2.74E−01 | 4.12E−02 | 2.55E−01 | 1.02E−01 |
| DTWD2 | NM_173666 | 3.64E−01 | 7.79E−02 | 1.18E−01 | 1.67E−01 | 4.03E−02 | 1.92E−01 |
| IHH | NM_002181 | 7.48E−02 | 2.73E−01 | 6.19E−02 | 1.43E−01 | 3.03E−01 | 1.04E−01 |
| NAA40 | NM_024771 | 2.53E−02 | 4.14E−01 | 5.32E−02 | 1.98E−01 | 1.16E−01 | 1.54E−01 |
| SPCS1 | NM_014041 | 2.19E−01 | 1.74E−02 | 1.64E−01 | 1.48E−01 | 3.67E−01 | 4.45E−02 |
| SEMA5A | NM_003966 | 2.34E−01 | 4.67E−01 | 9.50E−02 | 3.16E−02 | 4.50E−02 | 8.89E−02 |
| CA7 | NM_005182 | 5.81E−02 | 2.32E−01 | 1.63E−03 | 2.84E−01 | 2.23E−03 | 3.84E−01 |
| TNFRSF10A | NM_003844 | 7.63E−02 | 8.80E−02 | 1.48E−01 | 1.73E−01 | 2.21E−01 | 2.56E−01 |
| C2CD4C | NM_001136263 | 1.59E−01 | 1.74E−01 | 2.28E−01 | 1.40E−01 | 6.01E−02 | 2.03E−01 |
| CD72 | NM_001782 | 6.19E−02 | 1.58E−01 | 1.97E−02 | 2.64E−01 | 2.88E−01 | 1.71E−01 |
| VWA2 | NM_001272046 | 1.44E−01 | 1.43E−01 | 1.73E−01 | 1.83E−01 | 1.61E−01 | 1.60E−01 |
| DAOA | NM_001161812 | 6.39E−02 | 4.12E−01 | 1.15E−01 | 5.33E−02 | 2.25E−01 | 9.47E−02 |
| NKX3-2 | NM_001189 | 3.98E−01 | 2.15E−01 | 2.67E−02 | 1.31E−01 | 3.87E−02 | 1.54E−01 |
| IER3 | NM_003897 | 6.26E−02 | 4.59E−02 | 5.14E−02 | 3.57E−01 | 1.71E−01 | 2.79E−01 |
| OR51A7 | NM_001004749 | 5.20E−01 | 2.02E−02 | 2.14E−01 | 4.59E−02 | 5.79E−02 | 1.10E−01 |
| ARRDC3 | NM_020801 | 4.08E−01 | 3.26E−02 | 2.11E−01 | 2.81E−02 | 2.44E−01 | 4.38E−02 |
| C16orf59 | NM_025108 | 5.31E−02 | 3.38E−01 | 2.64E−02 | 2.27E−01 | 7.21E−02 | 2.51E−01 |
| CD274 | NM_014143 | 7.10E−02 | 2.35E−03 | 3.25E−01 | 9.87E−02 | 4.60E−01 | 1.06E−02 |
| EXOSC1 | NM_016046 | 2.40E−01 | 3.42E−01 | 1.25E−01 | 7.33E−02 | 5.35E−02 | 1.35E−01 |
| PELI1 | NM_020651 | 2.09E−01 | 1.53E−01 | 1.88E−01 | 1.72E−01 | 1.18E−01 | 1.28E−01 |
| ATG4A | NM_178270 | 3.62E−01 | 2.51E−01 | 6.12E−02 | 5.89E−02 | 1.61E−01 | 7.55E−02 |
| FAM228A | NM_001040710 | 1.71E−01 | 1.56E−01 | 1.62E−01 | 1.36E−01 | 2.00E−01 | 1.45E−01 |
| USP7 | NM_003470 | 2.37E−01 | 2.23E−01 | 6.12E−02 | 6.27E−02 | 2.83E−01 | 1.02E−01 |
| YOD1 | NM_001276320 | 2.36E−02 | 3.72E−01 | 7.98E−02 | 1.59E−01 | 6.02E−02 | 2.75E−01 |
| SLC52A2 | NM_024531 | 3.01E−01 | 3.60E−01 | 8.46E−02 | 1.11E−01 | 2.28E−02 | 8.95E−02 |
| PCDH11X | NM_001168360 | 5.99E−03 | 8.37E−02 | 2.74E−03 | 3.79E−01 | 1.55E−01 | 3.43E−01 |
| ASIC3 | NM_004769 | 4.52E−02 | 3.44E−01 | 2.66E−01 | 4.13E−02 | 2.20E−01 | 5.35E−02 |
| PLS1 | NM_001145319 | 1.70E−01 | 3.91E−01 | 3.23E−02 | 1.56E−01 | 7.25E−02 | 1.49E−01 |
| C17orf102 | NM_207454 | 5.28E−02 | 2.39E−01 | 5.92E−03 | 2.24E−01 | 1.97E−03 | 4.49E−01 |
| ARMC7 | NM_024585 | 1.09E−01 | 4.52E−02 | 8.37E−02 | 2.37E−01 | 2.52E−01 | 2.47E−01 |
| POU3F2 | NM_005604 | 6.10E−02 | 2.63E−01 | 1.47E−01 | 1.03E−01 | 6.61E−02 | 3.34E−01 |
| TFPI | NM_006287 | 1.25E−01 | 1.40E−01 | 1.88E−01 | 1.87E−01 | 1.16E−01 | 2.19E−01 |
| FAM83H | NM_198488 | 4.00E−01 | 1.65E−01 | 1.83E−01 | 4.60E−02 | 1.25E−01 | 5.47E−02 |
| ITGA7 | NM_002206 | 1.50E−01 | 1.41E−01 | 2.38E−02 | 1.79E−01 | 2.41E−01 | 2.39E−01 |
| B3GNT4 | NM_030765 | 1.70E−01 | 2.14E−01 | 2.09E−01 | 1.91E−01 | 1.31E−01 | 5.98E−02 |
| ARHGAP25 | NM_001166277 | 1.77E−01 | 1.56E−01 | 1.04E−01 | 2.37E−01 | 5.70E−02 | 2.44E−01 |
| LHX3 | NM_178138 | 4.02E−02 | 3.90E−01 | 8.07E−02 | 1.91E−01 | 4.70E−02 | 2.27E−01 |
| VHLL | NM_001004319 | 2.89E−01 | 3.33E−02 | 7.30E−02 | 2.19E−01 | 7.94E−02 | 2.83E−01 |
| KIAA1432 | NM_020829 | 2.32E−01 | 1.50E−01 | 1.72E−01 | 8.12E−02 | 1.67E−01 | 1.74E−01 |
| NGFRAP1 | NM_014380 | 8.06E−02 | 4.34E−01 | 2.58E−02 | 1.24E−01 | 1.79E−01 | 1.34E−01 |
| FXN | NM_001161706 | 1.56E−01 | 8.46E−02 | 3.73E−02 | 2.79E−01 | 1.05E−01 | 3.15E−01 |
| NTNG1 | NM_001113228 | 2.10E−01 | 1.09E−01 | 5.60E−02 | 2.70E−01 | 2.26E−02 | 3.11E−01 |
| PIK3R3 | NM_003629 | 8.43E−02 | 7.09E−03 | 1.72E−01 | 1.80E−01 | 1.55E−01 | 3.81E−01 |
| EVL | NM_016337 | 2.67E−02 | 1.52E−01 | 7.92E−02 | 3.74E−01 | 7.28E−02 | 2.75E−01 |
| LARS | NM_020117 | 1.08E−01 | 6.87E−03 | 2.91E−01 | 2.01E−02 | 5.41E−01 | 1.21E−02 |
| TOE1 | NM_025077 | 7.67E−02 | 7.34E−03 | 3.42E−01 | 1.93E−01 | 1.52E−01 | 2.08E−01 |
| KRT38 | NM_006771 | 6.12E−02 | 2.24E−01 | 2.25E−01 | 7.84E−02 | 2.94E−01 | 9.80E−02 |
| PRKD2 | NM_001079882 | 1.61E−01 | 2.52E−01 | 1.16E−01 | 2.29E−01 | 6.50E−02 | 1.59E−01 |
| FUNDC1 | NM_173794 | 5.79E−02 | 1.57E−01 | 9.02E−02 | 2.59E−01 | 1.56E−01 | 2.61E−01 |
| ALYREF | NM_005782 | 1.73E−01 | 4.65E−01 | 1.51E−02 | 1.14E−01 | 1.16E−01 | 9.98E−02 |
| KBTBD12 | NM_207335 | 9.84E−02 | 2.96E−01 | 2.06E−01 | 4.87E−02 | 2.67E−01 | 6.69E−02 |
| ADAL | NM_001159280 | 3.05E−02 | 7.47E−03 | 1.72E−01 | 3.10E−01 | 2.05E−01 | 2.58E−01 |
| P2RY10 | NM_014499 | 1.65E−01 | 8.35E−02 | 1.34E−01 | 1.58E−01 | 2.56E−01 | 1.86E−01 |
| NSUN4 | NM_001256128 | 2.58E−01 | 7.64E−02 | 2.85E−01 | 2.19E−02 | 2.59E−01 | 8.23E−02 |
| ANKRD45 | NM_198493 | 8.44E−02 | 6.64E−02 | 3.58E−01 | 1.21E−03 | 4.70E−01 | 3.37E−03 |
| HELZ2 | NM_033405 | 4.25E−02 | 2.02E−01 | 4.11E−02 | 1.61E−01 | 2.54E−01 | 2.84E−01 |
| NAP1L5 | NM_153757 | 5.52E−02 | 3.24E−01 | 2.55E−02 | 2.54E−01 | 2.78E−02 | 2.97E−01 |
| BABAM1 | NM_014173 | 1.43E−01 | 1.75E−01 | 1.00E−01 | 2.89E−01 | 6.82E−02 | 2.09E−01 |
| ADTRP | NM_032744 | 1.77E−02 | 3.10E−01 | 3.92E−02 | 2.92E−01 | 1.07E−01 | 2.19E−01 |
| AIF1 | NM_001623 | 2.09E−01 | 2.28E−01 | 7.12E−02 | 1.38E−01 | 1.98E−01 | 1.42E−01 |
| GPBP1 | NM_001127236 | 1.82E−01 | 4.73E−02 | 3.83E−01 | 2.17E−02 | 2.81E−01 | 7.07E−02 |
| RPS10 | NM_001203245 | 9.83E−02 | 4.20E−01 | 5.17E−02 | 1.95E−01 | 4.04E−02 | 1.83E−01 |
| C1orf162 | NM_174896 | 2.83E−01 | 2.46E−02 | 2.64E−01 | 4.35E−02 | 3.08E−01 | 6.47E−02 |
| SYNCRIP | NM_006372 | 1.43E−01 | 2.44E−01 | 1.54E−01 | 1.55E−01 | 6.65E−02 | 2.25E−01 |
| GRK4 | NM_182982 | 1.93E−01 | 4.96E−02 | 2.37E−01 | 3.25E−02 | 4.20E−01 | 5.66E−02 |
| TBC1D21 | NM_153356 | 1.67E−01 | 3.44E−01 | 1.60E−01 | 1.59E−01 | 1.10E−01 | 4.93E−02 |
| PDE4D | NM_001197222 | 6.50E−01 | 1.23E−01 | 7.51E−02 | 5.31E−02 | 2.17E−02 | 6.67E−02 |
| RPN1 | NM_002950 | 2.64E−01 | 2.43E−01 | 1.06E−01 | 9.25E−02 | 1.14E−01 | 1.72E−01 |
| FBXL19 | NM_001282351 | 4.46E−01 | 6.32E−02 | 1.91E−01 | 1.25E−01 | 1.43E−01 | 2.33E−02 |
| OR4S2 | NM_001004059 | 3.36E−02 | 5.50E−02 | 1.87E−02 | 4.19E−01 | 4.55E−02 | 4.19E−01 |
| ORC6 | NM_014321 | 6.02E−02 | 2.35E−01 | 5.57E−02 | 3.25E−01 | 3.90E−02 | 2.76E−01 |
| MRPL18 | NM_014161 | 3.15E−01 | 2.69E−01 | 2.08E−01 | 6.53E−02 | 9.71E−02 | 3.89E−02 |
| GALNT9 | NM_021808 | 2.21E−01 | 4.46E−01 | 5.12E−02 | 1.36E−01 | 2.22E−02 | 1.16E−01 |
| PIN4 | NM_001170747 | 3.10E−01 | 2.27E−02 | 2.42E−01 | 7.09E−02 | 2.87E−01 | 6.04E−02 |
| C2orf15 | NM_144706 | 2.52E−02 | 2.78E−01 | 5.60E−03 | 2.40E−01 | 3.44E−03 | 4.42E−01 |
| TUSC5 | NM_172367 | 6.25E−02 | 1.46E−01 | 2.80E−01 | 1.34E−01 | 3.62E−01 | 1.06E−02 |
| B4GALT2 | NM_003780 | 4.30E−01 | 4.39E−02 | 9.21E−02 | 1.88E−01 | 2.79E−02 | 2.14E−01 |
| NBL1 | NM_001278166 | 4.89E−02 | 3.29E−01 | 2.08E−01 | 7.42E−02 | 2.71E−01 | 6.53E−02 |
| AGK | NM_018238 | 2.15E−01 | 2.02E−01 | 1.01E−01 | 1.80E−01 | 8.70E−02 | 2.12E−01 |
| TCP11L1 | NM_001145541 | 9.13E−02 | 1.66E−01 | 2.12E−02 | 3.48E−01 | 8.29E−02 | 2.88E−01 |
| SOWAHC | NM_023016 | 2.09E−02 | 2.29E−01 | 1.34E−01 | 2.97E−01 | 1.06E−01 | 2.10E−01 |
| FOPNL | NM_144600 | 7.00E−02 | 6.40E−01 | 4.87E−02 | 8.91E−02 | 4.94E−02 | 1.01E−01 |
| NMNAT2 | NM_170706 | 2.67E−01 | 7.96E−02 | 1.21E−01 | 2.03E−01 | 9.40E−02 | 2.34E−01 |
| OR5P2 | NM_153444 | 2.06E−01 | 5.70E−02 | 3.01E−01 | 3.93E−02 | 3.23E−01 | 7.29E−02 |
| CPLX2 | NM_006650 | 3.74E−01 | 1.07E−01 | 3.93E−01 | 2.69E−02 | 7.53E−02 | 2.38E−02 |
| GNA13 | NM_006572 | 1.68E−01 | 3.57E−01 | 1.52E−01 | 3.77E−02 | 2.41E−01 | 4.36E−02 |
| PDXDC1 | NM_001285448 | 6.19E−02 | 2.82E−01 | 8.05E−02 | 1.39E−01 | 1.44E−01 | 2.92E−01 |
| NFE2L2 | NM_006164 | 7.58E−02 | 8.85E−02 | 4.07E−01 | 6.06E−02 | 2.63E−01 | 1.06E−01 |
| CYP2U1 | NM_183075 | 2.38E−01 | 3.07E−01 | 4.26E−02 | 1.38E−01 | 9.61E−02 | 1.80E−01 |
| PAGE5 | NM_001013435 | 2.94E−01 | 5.28E−02 | 2.72E−01 | 2.54E−02 | 3.28E−01 | 2.99E−02 |
| PVRL3 | NM_001243288 | 5.76E−02 | 4.75E−01 | 2.33E−02 | 1.12E−01 | 6.55E−02 | 2.69E−01 |
| SOAT1 | NM_001252511 | 1.68E−02 | 1.40E−01 | 1.49E−02 | 2.23E−01 | 1.18E−02 | 5.96E−01 |
| RRAGD | NM_021244 | 2.48E−02 | 5.44E−01 | 2.02E−02 | 1.59E−01 | 3.60E−02 | 2.19E−01 |
| KIF19 | NM_153209 | 3.44E−01 | 2.01E−02 | 3.09E−01 | 3.31E−02 | 2.00E−01 | 9.72E−02 |
| GUCY1A3 | NM_001130684 | 5.12E−02 | 8.15E−01 | 3.15E−03 | 2.66E−02 | 9.63E−04 | 1.07E−01 |
| PARP12 | NM_022750 | 1.25E−01 | 5.19E−01 | 5.87E−02 | 2.15E−01 | 3.84E−02 | 4.82E−02 |
| C2orf42 | NM_017880 | 1.81E−01 | 1.13E−01 | 1.96E−01 | 1.73E−01 | 1.32E−01 | 2.11E−01 |
| DPPA5 | NM_001025290 | 2.89E−02 | 1.78E−02 | 6.85E−02 | 4.08E−01 | 7.18E−02 | 4.12E−01 |
| NFASC | NM_001160331 | 1.58E−01 | 2.20E−01 | 2.63E−01 | 9.65E−02 | 1.67E−01 | 1.03E−01 |
| MED10 | NM_032286 | 1.20E−02 | 2.67E−01 | 2.44E−02 | 3.51E−01 | 2.08E−02 | 3.33E−01 |
| MSRB3 | NM_001193461 | 1.38E−01 | 3.83E−01 | 1.44E−01 | 7.20E−02 | 2.24E−01 | 4.59E−02 |
| RRP1B | NM_015056 | 1.80E−01 | 5.62E−02 | 3.74E−01 | 2.05E−03 | 3.90E−01 | 5.18E−03 |
| MAPKAP1 | NM_024117 | 7.91E−03 | 5.71E−01 | 7.03E−02 | 1.22E−01 | 1.01E−01 | 1.37E−01 |
| PDLIM2 | NM_021630 | 5.42E−01 | 1.50E−01 | 1.73E−01 | 4.68E−02 | 9.68E−03 | 8.75E−02 |
| TPSG1 | NM_012467 | 4.45E−02 | 1.64E−01 | 7.39E−02 | 7.53E−02 | 3.59E−01 | 2.93E−01 |
| STK32B | NM_018401 | 3.23E−02 | 1.03E−01 | 1.94E−01 | 2.90E−01 | 2.55E−01 | 1.36E−01 |
| VSIG4 | NM_001100431 | 2.40E−01 | 2.81E−01 | 2.03E−01 | 7.62E−02 | 1.16E−01 | 9.49E−02 |
| SIRT1 | NM_001142498 | 2.47E−01 | 5.85E−02 | 2.12E−01 | 3.49E−02 | 3.61E−01 | 9.74E−02 |
| TRAF3IP1 | NM_015650 | 9.03E−02 | 4.90E−03 | 3.33E−01 | 1.06E−02 | 5.31E−01 | 4.17E−02 |
| VILL | NM_015873 | 1.10E−01 | 4.67E−01 | 1.88E−01 | 2.03E−02 | 2.02E−01 | 2.35E−02 |
| PLCG1 | NM_002660 | 6.95E−02 | 1.76E−01 | 2.06E−01 | 1.13E−01 | 3.41E−01 | 1.07E−01 |
| TNXB | NM_032470 | 1.20E−01 | 1.05E−01 | 3.78E−02 | 5.08E−01 | 3.80E−02 | 2.03E−01 |
| SUMO2 | NM_006937 | 1.73E−01 | 1.64E−01 | 1.54E−01 | 1.74E−01 | 2.06E−01 | 1.41E−01 |
| OR10G7 | NM_001004463 | 2.54E−03 | 3.80E−01 | 3.58E−02 | 1.92E−01 | 2.28E−01 | 1.74E−01 |
| ARID2 | NM_152641 | 4.94E−01 | 4.97E−02 | 2.18E−01 | 3.95E−03 | 1.05E−01 | 1.45E−01 |
| CPEB1 | NM_001079533 | 2.10E−01 | 1.86E−01 | 4.76E−02 | 2.47E−01 | 2.73E−01 | 5.20E−02 |
| TNS4 | NM_032865 | 1.63E−01 | 2.28E−01 | 6.74E−02 | 2.60E−01 | 1.04E−01 | 1.94E−01 |
| MS4A6A | NM_152851 | 3.93E−01 | 2.82E−01 | 1.18E−01 | 8.77E−02 | 1.05E−01 | 3.24E−02 |
| DPF2 | NM_006268 | 1.76E−01 | 4.05E−01 | 1.55E−01 | 9.54E−02 | 1.29E−01 | 5.87E−02 |
| BRI3 | NM_015379 | 1.12E−01 | 2.95E−01 | 2.32E−02 | 2.41E−01 | 5.44E−02 | 2.94E−01 |
| PARD3 | NM_001184788 | 5.69E−02 | 5.55E−01 | 9.44E−03 | 9.15E−02 | 4.59E−02 | 2.61E−01 |
| AP5Z1 | NM_014855 | 2.02E−01 | 3.10E−02 | 4.67E−02 | 4.05E−01 | 1.05E−01 | 2.30E−01 |
| SLITRK6 | NM_032229 | 3.20E−02 | 4.29E−01 | 8.60E−02 | 1.07E−01 | 7.11E−02 | 2.95E−01 |
| MST4 | NM_001042452 | 3.78E−01 | 9.38E−02 | 1.83E−01 | 1.14E−01 | 1.76E−01 | 7.63E−02 |
| SMAD7 | NM_001190822 | 5.55E−02 | 5.67E−01 | 9.21E−02 | 5.82E−02 | 1.02E−01 | 1.47E−01 |
| YES1 | NM_005433 | 1.53E−03 | 1.06E−01 | 6.65E−02 | 3.12E−01 | 1.41E−01 | 3.94E−01 |
| CHRD | NM_003741 | 1.32E−01 | 1.82E−01 | 2.38E−01 | 1.86E−01 | 1.44E−01 | 1.41E−01 |
| TNIP2 | NM_024309 | 2.84E−01 | 2.48E−01 | 6.98E−02 | 1.98E−01 | 2.61E−03 | 2.20E−01 |
| ZRANB2 | NM_005455 | 1.27E−01 | 1.23E−01 | 2.45E−01 | 7.42E−02 | 3.30E−01 | 1.24E−01 |
| KIAA0586 | NM_001244189 | 3.44E−01 | 7.65E−02 | 2.86E−02 | 2.02E−01 | 8.60E−02 | 2.86E−01 |
| USPL1 | NM_005800 | 2.85E−02 | 2.89E−01 | 2.24E−03 | 3.16E−01 | 1.35E−01 | 2.52E−01 |
| HCRTR2 | NM_001526 | 2.77E−01 | 8.07E−04 | 2.96E−01 | 1.70E−02 | 4.08E−01 | 2.47E−02 |
| ZNF397 | NM_001135178 | 2.19E−01 | 6.96E−02 | 1.25E−01 | 6.32E−02 | 2.29E−01 | 3.18E−01 |
| FAM53B | NM_014661 | 2.72E−01 | 1.42E−02 | 2.81E−01 | 6.29E−03 | 4.36E−01 | 1.40E−02 |
| LYAR | NM_017816 | 1.01E−01 | 6.28E−01 | 2.99E−02 | 6.13E−02 | 3.56E−02 | 1.68E−01 |
| CAV1 | NM_001172896 | 2.61E−01 | 2.50E−01 | 1.34E−01 | 1.86E−01 | 8.97E−02 | 1.04E−01 |
| TMEM89 | NM_001008269 | 4.06E−01 | 1.73E−02 | 3.26E−01 | 5.87E−03 | 2.56E−01 | 1.30E−02 |
| SPPL2C | NM_175882 | 1.95E−01 | 4.46E−02 | 2.41E−01 | 1.24E−01 | 3.56E−01 | 6.50E−02 |
| ATE1 | NM_001288736 | 3.04E−01 | 5.92E−02 | 2.54E−01 | 1.66E−01 | 1.07E−01 | 1.36E−01 |
| CLDN23 | NM_194284 | 1.39E−01 | 2.74E−01 | 4.25E−02 | 2.75E−01 | 3.83E−02 | 2.58E−01 |
| PLEKHG1 | NM_001029884 | 1.48E−01 | 1.11E−01 | 2.20E−01 | 2.10E−01 | 1.80E−01 | 1.59E−01 |
| ETFDH | NM_001281738 | 7.58E−02 | 1.13E−01 | 4.08E−02 | 2.87E−01 | 1.53E−01 | 3.59E−01 |
| ISYNA1 | NM_016368 | 4.61E−02 | 1.97E−01 | 2.69E−01 | 7.86E−02 | 3.36E−01 | 1.03E−01 |
| CAV1 | NM_001172897 | 1.87E−01 | 3.20E−01 | 8.99E−02 | 1.33E−01 | 1.75E−01 | 1.24E−01 |
| EMILIN3 | NM_052846 | 2.25E−01 | 1.04E−02 | 2.84E−01 | 1.88E−02 | 3.76E−01 | 1.16E−01 |
| ASB15 | NM_080928 | 1.37E−01 | 2.23E−01 | 5.79E−02 | 9.37E−02 | 3.97E−01 | 1.21E−01 |
| IFNA2 | NM_000605 | 4.56E−02 | 2.50E−01 | 7.82E−02 | 2.19E−01 | 2.28E−01 | 2.09E−01 |
| RRAS | NM_006270 | 1.33E−01 | 3.17E−01 | 1.70E−01 | 1.73E−01 | 4.76E−02 | 1.92E−01 |
| SHANK3 | NM_033517 | 1.55E−02 | 5.16E−01 | 3.73E−02 | 1.46E−01 | 1.34E−01 | 1.83E−01 |
| TGIF1 | NM_173211 | 3.70E−01 | 1.54E−01 | 1.61E−01 | 6.78E−02 | 1.72E−01 | 1.06E−01 |
| SFRP2 | NM_003013 | 7.58E−02 | 1.71E−01 | 3.73E−01 | 1.01E−01 | 3.06E−01 | 5.11E−03 |
| CSRP2BP | NM_020536 | 1.61E−01 | 2.43E−01 | 1.40E−01 | 2.16E−01 | 8.39E−02 | 1.88E−01 |
| RBMXL2 | NM_014469 | 7.58E−02 | 1.26E−01 | 4.25E−01 | 2.01E−04 | 4.05E−01 | 4.33E−04 |
| GH2 | NM_022556 | 3.57E−01 | 1.21E−01 | 4.98E−02 | 1.57E−01 | 1.50E−01 | 1.98E−01 |
| GP1BB | NM_000407 | 4.93E−02 | 5.89E−01 | 3.49E−02 | 2.33E−01 | 3.89E−02 | 8.86E−02 |
| RAB9B | NM_016370 | 7.06E−01 | 3.75E−03 | 1.31E−01 | 3.35E−02 | 1.19E−01 | 3.97E−02 |
| IRX1 | NM_024337 | 2.72E−02 | 3.86E−02 | 3.92E−01 | 7.08E−02 | 4.46E−01 | 5.96E−02 |
| KLK13 | NM_015596 | 2.49E−01 | 2.19E−01 | 1.23E−01 | 1.63E−01 | 8.45E−02 | 1.96E−01 |
| CBX3 | NM_016587 | 6.49E−01 | 5.27E−03 | 1.85E−01 | 6.56E−02 | 6.04E−02 | 6.88E−02 |
| NFKB1 | NM_001165412 | 4.58E−01 | 4.78E−03 | 1.66E−01 | 9.85E−02 | 1.12E−01 | 1.94E−01 |
| PPP1R3F | NM_033215 | 2.44E−01 | 2.65E−02 | 2.67E−01 | 7.23E−02 | 3.40E−01 | 8.43E−02 |
| CCDC9 | NM_015603 | 5.97E−02 | 9.42E−02 | 3.02E−01 | 7.05E−02 | 4.91E−01 | 1.82E−02 |
| ORC4 | NM_002552 | 1.48E−01 | 3.45E−01 | 1.42E−01 | 2.56E−01 | 8.25E−02 | 6.20E−02 |
| PLEC | NM_201378 | 1.56E−01 | 6.88E−02 | 2.51E−01 | 1.39E−01 | 2.48E−01 | 1.76E−01 |
| WDR5 | NM_052821 | 1.47E−01 | 4.72E−01 | 1.10E−01 | 1.02E−01 | 1.06E−01 | 1.01E−01 |
| CA2 | NM_000067 | 4.12E−01 | 3.63E−02 | 3.27E−01 | 1.14E−02 | 2.00E−01 | 5.11E−02 |
| VANGL1 | NM_138959 | 7.11E−02 | 3.31E−01 | 1.42E−01 | 1.10E−01 | 1.45E−01 | 2.39E−01 |
| FOXK2 | NM_004514 | 1.53E−01 | 3.15E−01 | 1.76E−01 | 1.17E−01 | 1.56E−01 | 1.23E−01 |
| STEAP1B | NM_207342 | 3.24E−01 | 2.28E−01 | 2.16E−01 | 2.35E−02 | 2.33E−01 | 1.37E−02 |
| TRIM48 | NM_024114 | 2.49E−01 | 7.57E−02 | 2.64E−01 | 1.13E−01 | 3.28E−01 | 9.26E−03 |
| VMAC | NM_001017921 | 4.61E−01 | 2.28E−01 | 1.65E−01 | 6.33E−02 | 9.58E−02 | 2.64E−02 |
| PSME2 | NM_002818 | 4.36E−02 | 1.95E−01 | 1.12E−01 | 2.96E−01 | 9.75E−02 | 2.96E−01 |
| ZNF275 | NM_001080485 | 3.10E−01 | 5.57E−03 | 3.56E−01 | 1.97E−02 | 3.32E−01 | 1.71E−02 |
| CAPRIN1 | NM_203364 | 1.85E−01 | 4.27E−01 | 1.37E−01 | 7.35E−02 | 1.10E−01 | 1.07E−01 |
| ADCK2 | NM_052853 | 2.52E−01 | 2.97E−01 | 1.86E−01 | 1.73E−02 | 2.70E−01 | 1.90E−02 |
| DNAH12 | NM_178504 | 2.76E−02 | 3.13E−01 | 5.97E−02 | 3.58E−01 | 2.04E−01 | 7.92E−02 |
| WBSCR27 | NM_152559 | 4.16E−01 | 3.77E−03 | 2.11E−01 | 3.18E−02 | 3.41E−01 | 3.99E−02 |
| BRINP1 | NM_014618 | 3.89E−01 | 5.28E−02 | 1.05E−01 | 1.07E−01 | 2.32E−01 | 1.58E−01 |
| ZNF331 | NM_001079906 | 1.05E−02 | 6.71E−02 | 7.09E−02 | 3.88E−01 | 9.00E−02 | 4.17E−01 |
| UBXN1 | NM_001286078 | 4.08E−02 | 5.67E−01 | 1.12E−02 | 2.66E−01 | 5.71E−03 | 1.53E−01 |
| ART5 | NM_001079536 | 1.55E−02 | 1.17E−01 | 2.29E−01 | 2.14E−01 | 2.20E−01 | 2.49E−01 |
| IMPDH1 | NM_001142574 | 3.04E−01 | 4.09E−02 | 1.84E−01 | 9.51E−02 | 3.07E−01 | 1.12E−01 |
| HAUS8 | NM_033417 | 9.22E−02 | 1.52E−01 | 5.73E−01 | 5.74E−02 | 1.09E−01 | 6.10E−02 |
| ARFIP2 | NM_012402 | 3.67E−01 | 4.97E−02 | 2.06E−01 | 9.44E−02 | 2.06E−01 | 1.22E−01 |
| KRI1 | NM_023008 | 1.52E−01 | 1.35E−01 | 2.31E−01 | 1.89E−01 | 2.62E−01 | 7.75E−02 |
| TOP3B | NM_001282113 | 1.27E−01 | 2.07E−02 | 5.16E−02 | 3.27E−01 | 1.80E−01 | 3.39E−01 |
| COL5A2 | NM_000393 | 1.31E−01 | 3.31E−01 | 2.61E−01 | 7.67E−02 | 2.33E−01 | 1.28E−02 |
| PPP1R3D | NM_006242 | 3.29E−01 | 1.37E−01 | 1.66E−01 | 1.10E−01 | 2.14E−01 | 8.99E−02 |
| OR6N2 | NM_001005278 | 1.09E−01 | 2.65E−01 | 2.56E−01 | 3.67E−02 | 3.19E−01 | 6.08E−02 |
| GLTSCR2 | NM_015710 | 5.13E−01 | 1.27E−01 | 9.58E−02 | 7.54E−02 | 1.23E−01 | 1.13E−01 |
| SLC1A3 | NM_001166695 | 3.51E−01 | 5.00E−02 | 2.27E−01 | 1.57E−01 | 9.14E−02 | 1.70E−01 |
| DOCK11 | NM_144658 | 1.44E−01 | 3.24E−01 | 6.02E−02 | 2.47E−01 | 6.37E−02 | 2.08E−01 |
| RNPC3 | NM_017619 | 6.16E−02 | 1.56E−01 | 1.15E−01 | 1.61E−01 | 1.87E−01 | 3.66E−01 |
| VPS37A | NM_001145152 | 4.27E−02 | 9.79E−01 | 5.51E−03 | 1.45E−02 | 1.84E−03 | 3.71E−03 |
| PEX3 | NM_003630 | 3.16E−01 | 4.58E−01 | 6.01E−02 | 1.28E−01 | 5.31E−02 | 3.24E−02 |
| MAL | NM_022440 | 6.01E−01 | 8.87E−02 | 2.20E−01 | 2.08E−02 | 1.12E−01 | 5.71E−03 |
| OR51T1 | NM_001004759 | 5.26E−01 | 2.17E−01 | 1.40E−01 | 9.04E−02 | 3.86E−02 | 3.68E−02 |
| RORC | NM_001001523 | 1.56E−01 | 2.27E−01 | 6.62E−02 | 1.49E−01 | 2.20E−01 | 2.32E−01 |
| URI1 | NM_001252641 | 3.72E−02 | 2.66E−01 | 1.64E−01 | 1.93E−01 | 1.62E−01 | 2.28E−01 |
| NR2F2 | NM_021005 | 3.02E−01 | 2.86E−01 | 4.15E−02 | 2.08E−01 | 5.34E−02 | 1.59E−01 |
| CGB7 | NM_033142 | 1.30E−01 | 2.38E−01 | 1.81E−01 | 1.94E−01 | 1.72E−01 | 1.35E−01 |
| BCL2A1 | NM_001114735 | 9.39E−02 | 5.87E−03 | 2.14E−01 | 2.20E−01 | 1.25E−01 | 3.92E−01 |
| ZNF215 | NM_013250 | 1.88E−01 | 1.64E−01 | 1.05E−02 | 3.31E−01 | 1.64E−02 | 3.41E−01 |
| INA | NM_032727 | 1.40E−02 | 2.67E−01 | 6.14E−03 | 3.77E−01 | 3.02E−03 | 3.85E−01 |
| FAM69A | NM_001252271 | 5.90E−02 | 3.22E−01 | 1.46E−01 | 1.66E−01 | 2.44E−01 | 1.14E−01 |
| EPHX4 | NM_173567 | 3.87E−02 | 2.16E−01 | 4.60E−02 | 3.71E−01 | 3.75E−02 | 3.43E−01 |
| ITPKB | NM_002221 | 7.08E−02 | 4.15E−01 | 5.04E−02 | 3.03E−01 | 3.80E−02 | 1.75E−01 |
| C10orf25 | NM_001039380 | 7.29E−01 | 2.03E−01 | 3.72E−02 | 2.74E−02 | 1.37E−02 | 4.28E−02 |
| FRZB | NM_001463 | 1.81E−01 | 3.70E−01 | 1.32E−01 | 1.63E−01 | 7.63E−02 | 1.31E−01 |
| SMIM15 | NM_001048249 | 2.71E−01 | 2.18E−01 | 1.54E−01 | 7.75E−02 | 9.15E−02 | 2.43E−01 |
| IPCEF1 | NM_015553 | 4.80E−02 | 1.17E−01 | 1.21E−01 | 1.06E−01 | 5.41E−01 | 1.20E−01 |
| SPINK2 | NM_021114 | 3.12E−01 | 1.00E−01 | 2.18E−01 | 2.17E−01 | 1.66E−01 | 4.21E−02 |

TABLE 2
Negative control genes and candidate genes enriched in the CRISPRa screen. Set of 311
negative control housekeeping genes for estimating FDR of screening results and evaluating
cytolytic activity. Negative control genes consisted of ribosomal proteins, RNA polymerases,
translation factors, mitochondrial ribosomal proteins, GAPDH, and ACTB. Candidate genes
were enriched in the top 1% across at least two screening replicates.

| Negative | Negative | Candidate | Candidate | Candidate | Candidate |
|---|---|---|---|---|---|
| Control | Control | Gene | Gene | Gene | Gene |
| Name | ID | Name | Refseq | Name | Refseq |
| ACTB | NM 001101 | ABCC8 | NM 001287174 | MAP4K4 | NM 001242559 |
| EIF1 | NM 005801 | ABHD5 | NM 016006 | MAPK11 | NM 002751 |
| EIF1AD | NM 001242484 | ABHD8 | NM 024527 | MAPRE2 | NM 014268 |
| EIF1AX | NM 001412 | ACACA | NM 198839 | MBD3L2 | NM 144614 |
| EIF1AY | NM 004681 | ACOT6 | NM 001037162 | MBD3L3 | NM 001164425 |
| EIF1B | NM_005875 | ACP6 | NM_016361 | MBLAC1 | NM_203397 |
| EIF2A | NM 032025 | ACSL1 | NM 001286708 | MCAM | NM 006500 |
| EIF2AK1 | NM_001134335 | ACTR2 | NM_005722 | MCL1 | NM_021960 |
| EIF2AK2 | NM 002759 | ADAMTS12 | NM 030955 | MCU | NM 138357 |
| EIF2AK2 | NM 001135652 | ADAMTS13 | NM 139027 | MDK | NM 001270550 |
| EIF2AK3 | NM_004836 | AFAP1L1 | NM_001146337 | ME2 | NM_001168335 |
| EIF2AK4 | NM 001013703 | AMER1 | NM 152424 | MEFV | NM 001198536 |
| EIF2B1 | NM_001414 | AMER3 | NM_001105193 | MEIS2 | NM_172315 |
| EIF2B2 | NM 014239 | AMER3 | NM 001105195 | MEN1 | NM 000244 |
| EIF2B3 | NM 001261418 | ANGEL2 | NM 144567 | MFSD6 | NM 017694 |
| EIF2B4 | NM 172195 | ANGPTL7 | NM 021146 | MGAT5B | NM 001199172 |
| EIF2B4 | NM 015636 | ANKAR | NM 144708 | MINPP1 | NM 001178118 |
| EIF2B5 | NM_003907 | ANKEF1 | NM_198798 | MKNK2 | NM_017572 |
| EIF2D | NM 006893 | ANKRD45 | NM 198493 | MLNR | NM 001507 |
| EIF2S1 | NM_004094 | ANKRD52 | NM_173595 | MOCS1 | NM_001075098 |
| EIF2S2 | NM 003908 | AP1B1 | NM 001127 | MPC1L | NM 001195522 |
| EIF2S3 | NM 001415 | AP3M1 | NM 207012 | MPP2 | NM 001278371 |
| EIF3A | NM_003750 | APC | NM_001127510 | MPZL2 | NM_005797 |
| EIF3B | NM 003751 | APLP2 | NM 001243299 | MRPL15 | NM 014175 |
| EIF3C | NM_001037808 | AQP1 | NM_198098 | MRPL53 | NM_053050 |
| EIF3C | NM 001199142 | AOP8 | NM 001169 | MSS51 | NM 001024593 |
| EIF3CL | NM 001099661 | ARFGEF1 | NM 006421 | MXI1 | NM 130439 |
| EIF3D | NM 003753 | ARHGAP27 | NM 001282290 | MYBL1 | NM 001144755 |
| EIF3E | NM 001568 | ARHGAP27 | NM 174919 | MYL9 | NM 006097 |
| EIF3F | NM_003754 | ARHGAP28 | NM_001010000 | MZF1 | NM_001267033 |
| EIF3G | NM 003755 | ARHGEF10L | NM 018125 | NAT8L | NM 178557 |
| EIF3H | NM 003756 | ARID3C | NM 001017363 | NBL1 | NM 001204086 |
| EIF3I | NM 003757 | ARL6IP5 | NM 006407 | NBL1 | NM 001278165 |
| EIF3J | NM 003758 | ARMCX5 | NM 001168478 | NBPF15 | NM 173638 |
| EIF3K | NM_013234 | ARMCX5 | NM_001168480 | NCOA7 | NM_001199619 |
| EIF3L | NM 001242923 | ARNT | NM 001668 | NEDD4L | NM 001144966 |
| EIF3M | NM_006360 | ARPP21 | NM_001267617 | NEDD4L | NM_001144968 |
| EIF4A1 | NM 001416 | ARPP21 | NM 001267619 | NEK6 | NM 001166169 |
| EIF4A2 | NM 001967 | ARRDC5 | NM 001080523 | NEK6 | NM 001166171 |
| EIF4A3 | NM_014740 | ASXL3 | NM_030632 | NFATC1 | NM_001278670 |
| EIF4B | NM 001417 | ATG3 | NM 022488 | NFE2L2 | NM 001145413 |
| EIF4E | NM_001130678 | ATP2A2 | NM_170665 | NIFK | NM_032390 |
| EIF4E | NM 001968 | ATP2C1 | NM 001001485 | NIPAL4 | NM 001172292 |
| EIF4E1B | NM 001099408 | ATP2C1 | NM 001001487 | NME1 | NM 000269 |
| EIF4E2 | NM 001276337 | ATP5S | NM 015684 | NME2 | NM 001018139 |
| EIF4E3 | NM 001134650 | B3GAT1 | NM 018644 | NOL9 | NM 024654 |
| EIF4E3 | NM_001134651 | B3GNT2 | NM_006577 | NSG1 | NM_001287763 |
| EIF4E3 | NM 001134649 | BCAR1 | NM 001170717 | NSG1 | NM 014392 |
| EIF4E3 | NM_173359 | BCL2A1 | NM_001114735 | NSMCE4A | NM_001167865 |
| EIF4E3 | NM 001282886 | BCOR | NM 001123385 | NUDT14 | NM 177533 |
| EIF4EBP1 | NM 004095 | BMP5 | NM 021073 | NUDT18 | NM 024815 |
| EIF4EBP2 | NM_004096 | BMX | NM_001721 | NXT2 | NM_018698 |
| EIF4EBP3 | NM 003732 | BMX | NM 203281 | ODF3 | NM 001286136 |
| EIF4ENIF1 | NM_001164501 | BPIFB3 | NM_182658 | ODF3L2 | NM_182577 |
| EIF4ENIF1 | NM 001164502 | BSG | NM 198589 | OGG1 | NM 002542 |
| EIF4G1 | NM 182917 | BSG | NM 198591 | OLAH | NM 018324 |
| EIF4G1 | NM_001291157 | BTAF1 | NM_003972 | OLFM1 | NM_006334 |
| EIF4G1 | NM 004953 | BTC | NM 001729 | OR13C2 | NM 001004481 |
| EIF4G1 | NM_198244 | BTNL10 | NM_001287262 | OR4C12 | NM_001005270 |
| EIF4G2 | NM 001172705 | C12orf29 | NM 001009894 | OR51L1 | NM 001004755 |
| EIF4G2 | NM 001418 | C12orf5 | NM 020375 | OR5AN1 | NM 001004729 |
| EIF4G3 | NM 003760 | C12orf74 | NM 001178097 | ORAI3 | NM 152288 |
| EIF4G3 | NM 001198803 | C16orf70 | NM 025187 | OTOP3 | NM 178233 |
| EIF4G3 | NM_001198802 | C16orf90 | NM_001080524 | OTX1 | NM_014562 |
| EIF4H | NM 022170 | C16orf93 | NM 001014979 | OVCH1 | NM 183378 |
| EIF5 | NM_001969 | C17orf102 | NM_207454 | OXR1 | NM_001198532 |
| EIF5 | NM 183004 | C19orf52 | NM 138358 | P2RX5 | NM 001204519 |
| EIF5A | NM 001143761 | C1orf146 | NM 001012425 | P2RX6 | NM 001159554 |
| EIF5A | NM_001143762 | C1orf64 | NM_178840 | P4HA3 | NM_182904 |
| EIF5A | NM 001143760 | C1orf87 | NM 152377 | PAM16 | NM 016069 |
| EIF5A | NM_001970 | C1R | NM_001733 | PBX3 | NM_006195 |
| EIF5A2 | NM 020390 | C20orf78 | NM 001242671 | PCBP1 | NM 006196 |
| EIF5AL1 | NM_001099692 | C20orf96 | NM_153269 | PCDH11X | NM_001168360 |
| EIF5B | NM 015904 | C2orf15 | NM 144706 | PCDHA12 | NM 018903 |
| EIF6 | NM_181468 | C2orf76 | NM_001017927 | PCDHGA1 | NM_018912 |
| EIF6 | NM 181466 | C2orf88 | NM 001042520 | PCP4 | NM 006198 |
| GAPDH | NM_001289746 | C2orf88 | NM_001042521 | PCSK9 | NM_174936 |
| GAPDH | NM 002046 | C5orf55 | NM 138464 | PCYOX1L | NM 024028 |
| GAPDH | NM 001256799 | C7orf76 | NM 001201451 | PDCL2 | NM 152401 |
| MRPL1 | NM_020236 | C8orf22 | NM_001256598 | PDE1A | NM_001003683 |
| MRPL10 | NM 148887 | C8orf33 | NM 023080 | PDE1A | NM 001258314 |
| MRPL11 | NM_016050 | C8orf76 | NM_032847 | PDE1A | NM_005019 |
| MRPL12 | NM 002949 | C8orf86 | NM 207412 | PDE4C | NM 001098818 |
| MRPL13 | NM 014078 | C9orf40 | NM 017998 | PDIA3 | NM 005313 |
| MRPL14 | NM 032111 | C9orf50 | NM 199350 | PDK2 | NM 001199898 |
| MRPL15 | NM 014175 | CA7 | NM 005182 | PDK2 | NM 001199900 |
| MRPL16 | NM_017840 | CAB39L | NM_001287339 | PEG3 | NM_001146186 |
| MRPL17 | NM 022061 | CALM1 | NM 006888 | PGM2L1 | NM 173582 |
| MRPL18 | NM_014161 | CAPNS2 | NM_032330 | PHF11 | NM_001040443 |
| MRPL19 | NM 014763 | CARD10 | NM 014550 | PIANP | NM 001244015 |
| MRPL2 | NM 015950 | CARHSP1 | NM 001042476 | PIGO | NM 001201484 |
| MRPL20 | NM_017971 | CATSPERG | NM_021185 | PIGZ | NM_025163 |
| MRPL21 | NM 181515 | CCDC66 | NM 001141947 | PITPNB | NM 001284278 |
| MRPL22 | NM_014180 | CCNC | NM_005190 | PITRM1 | NM_001242307 |
| MRPL23 | NM 021134 | CCR4 | NM 005508 | PITX1 | NM 002653 |
| MRPL24 | NM 024540 | CCSER2 | NM 001284242 | PLCD1 | NM 001130964 |
| MRPL27 | NM 016504 | CD14 | NM 001174105 | PLEKHG2 | NM 022835 |
| MRPL28 | NM 006428 | CD164L2 | NM 207397 | PLEKHG5 | NM 001042663 |
| MRPL3 | NM_007208 | CD200R1L | NM_001199215 | PLEKHM3 | NM_001080475 |
| MRPL30 | NM 145212 | CD274 | NM 014143 | PLN | NM 002667 |
| MRPL32 | NM 031903 | CD36 | NM 001127444 | PNLIPRP1 | NM 006229 |
| MRPL33 | NM 004891 | CD36 | NM 001289908 | POC1B | NM 001199777 |
| MRPL34 | NM 023937 | CD3G | NM 000073 | POLD3 | NM 006591 |
| MRPL35 | NM_145644 | CDC14A | NM_033312 | POLR3GL | NM_032305 |
| MRPL36 | NM 032479 | CDC20 | NM 001255 | POP7 | NM 005837 |
| MRPL37 | NM_016491 | CDC42BPB | NM_006035 | POU3F1 | NM_002699 |
| MRPL38 | NM 032478 | CDH18 | NM 001167667 | PPME1 | NM 001271593 |
| MRPL39 | NM 017446 | CDK10 | NM 052988 | PPOX | NM 000309 |
| MRPL4 | NM_015956 | CDK19 | NM_015076 | PPP1R18 | NM_001134870 |
| MRPL40 | NM 003776 | CDK2AP2 | NM 001271849 | PPP1R8 | NM 002713 |
| MRPL41 | NM_032477 | CDKN1A | NM_001220778 | PPRC1 | NM_015062 |
| MRPL42 | NM 172177 | CDKN2A | NM 000077 | PQBP1 | NM 001167989 |
| MRPL43 | NM 176792 | CDYL | NM 004824 | PQBP1 | NM 001167990 |
| MRPL44 | NM 022915 | CEBPE | NM 001805 | PRDM2 | NM 001007257 |
| MRPL45 | NM 001278279 | CELA2B | NM 015849 | PRDX1 | NM 001202431 |
| MRPL46 | NM_022163 | CEP41 | NM_001257159 | PRDX6 | NM_004905 |
| MRPL47 | NM 020409 | CGREF1 | NM 001166241 | PRH1 | NM 001291314 |
| MRPL48 | NM_016055 | CHCHD10 | NM_213720 | PRICKLE1 | NM_001144883 |
| MRPL49 | NM 004927 | CHID1 | NM 001142676 | PRKACB | NM 002731 |
| MRPL50 | NM 019051 | CHRNA10 | NM 020402 | PRKAG1 | NM 001206710 |
| MRPL51 | NM_016497 | CHRNA9 | NM_017581 | PRKAG1 | NM_002733 |
| MRPL52 | NM 181306 | CHST13 | NM 152889 | PROCA1 | NM 152465 |
| MRPL53 | NM_053050 | CHTOP | NM_015607 | PSMF1 | NM_006814 |
| MRPL54 | NM 172251 | CIART | NM 144697 | PSMF1 | NM 178578 |
| MRPL55 | NM 181456 | CIDEB | NM 014430 | PSMG3 | NM 032302 |
| MRPL57 | NM_024026 | CIDEC | NM_001199623 | PTGR1 | NM_001146109 |
| MRPL9 | NM 031420 | CLDN14 | NM 001146077 | PTK2 | NM 001199649 |
| MRPS10 | NM_018141 | CLDN17 | NM_012131 | PTPDC1 | NM_001253830 |
| MRPS11 | NM 176805 | CLDN8 | NM 199328 | PTPDC1 | NM 177995 |
| MRPS12 | NM 021107 | CLEC2L | NM 001080511 | PTPMT1 | NM 001143984 |
| MRPS12 | NM 033362 | CLIP3 | NM 001199570 | PTTG1IP | NM 004339 |
| MRPS14 | NM 022100 | CLIP3 | NM 015526 | PWP1 | NM 007062 |
| MRPS15 | NM_031280 | COL10A1 | NM_000493 | R3HDM1 | NM_001282799 |
| MRPS16 | NM 016065 | COL20A1 | NM 020882 | RAB43 | NM 001204885 |
| MRPS17 | NM_015969 | COL25A1 | NM_001256074 | RABEPK | NM_001174153 |
| MRPS18A | NM 001193343 | COL4A3 | NM 000091 | RABGAP1L | NM 001035230 |
| MRPS18B | NM 014046 | COMMD4 | NM 017828 | RABGAP1L | NM 001243764 |
| MRPS18C | NM_016067 | CORO2B | NM_006091 | RABGGTA | NM_182836 |
| MRPS2 | NM 016034 | COX10 | NM 001303 | RAD51AP2 | NM 001099218 |
| MRPS21 | NM_018997 | COX14 | NM_001257134 | RALGDS | NM_001271774 |
| MRPS22 | NM 020191 | CPLX3 | NM 001030005 | RALGDS | NM 001271775 |
| MRPS23 | NM_016070 | CPN2 | NM_001080513 | RASA2 | NM_006506 |
| MRPS24 | NM 032014 | CRB3 | NM 174881 | RASD2 | NM 014310 |
| MRPS25 | NM_022497 | CREG2 | NM_153836 | RASSF3 | NM_178169 |
| MRPS26 | NM_030811 | CREM | NM 183012 | RBBP8 | NM 002894 |
| MRPS27 | NM_001286751 | CSMD2 | NM_052896 | RBM24 | NM_001143941 |
| MRPS28 | NM 014018 | CST2 | NM 001322 | RBMXL2 | NM 014469 |
| MRPS30 | NM 016640 | CST4 | NM 001899 | RBPMS2 | NM 194272 |
| MRPS31 | NM_005830 | CSTF1 | NM_001033521 | RCAN2 | NM_001251974 |
| MRPS33 | NM 053035 | CTBP1 | NM 001012614 | RFC2 | NM 002914 |
| MRPS33 | NM_016071 | CXorf57 | NM_018015 | RFNG | NM_002917 |
| MRPS34 | NM 023936 | CYB5D2 | NM 001254755 | RHCG | NM 016321 |
| MRPS35 | NM 001190864 | CYB5RL | NM 001031672 | RHOH | NM 001278369 |
| MRPS36 | NM 033281 | DAAM2 | NM 001201427 | RIC8B | NM 018157 |
| MRPS5 | NM 031902 | DAAM2 | NM 015345 | RIIAD1 | NM 001144956 |
| MRPS6 | NM_032476 | DAB2IP | NM_032552 | RIMKLB | NM_020734 |
| MRPS7 | NM 015971 | DCN | NM 001920 | RINT1 | NM 021930 |
| MRPS9 | NM_182640 | DCP1A | NM_001290204 | RNF146 | NM_001242851 |
| POLR1A | NM 015425 | DCTN2 | NM 001261413 | RNF165 | NM 152470 |
| POLR1B | NM 001282772 | DDHD2 | NM 001164234 | RNF186 | NM 019062 |
| POLR1B | NM_001282776 | DDIT4L | NM_145244 | RNF213 | NM_001256071 |
| POLR1C | NM 203290 | DDX43 | NM 018665 | RNLS | NM 018363 |
| POLR1D | NM_015972 | DEFB127 | NM_139074 | RPA1 | NM_002945 |
| POLR1D | NM 001206559 | DKK2 | NM 014421 | RPL15 | NM 001253384 |
| POLR1E | NM 001282766 | DNAJB1 | NM 006145 | RPL6 | NM 000970 |
| POLR2A | NM 000937 | DNPEP | NM 012100 | RPLP1 | NM 213725 |
| POLR2B | NM 000938 | DPF1 | NM 004647 | RPN2 | NM 001135771 |
| POLR2C | NM_032940 | DPM3 | NM_153741 | RPS27A | NM_001135592 |
| POLR2D | NM 004805 | DSEL | NM 032160 | RPUSD4 | NM 032795 |
| POLR2E | NM 002695 | DUPD1 | NM 001003892 | RRP1B | NM 015056 |
| POLR2F | NM 021974 | DUT | NM 001025249 | RXRA | NM 002957 |
| POLR2G | NM 002696 | DUT | NM 001948 | RXRG | NM 001256571 |
| POLR2H | NM_001278715 | ECHDC1 | NM_018479 | SAMD1 | NM_138352 |
| POLR2H | NM 001278700 | EFHC1 | NM 001172420 | SAMD3 | NM 001258275 |
| POLR2I | NM_006233 | EFHC1 | NM_018100 | SATB1 | NM_001195470 |
| POLR2J | NM 006234 | EFHD1 | NM 025202 | SBSN | NM 001166034 |
| POLR2J2 | NM 032959 | EFNA1 | NM 004428 | SCP2D1 | NM 178483 |
| POLR2J3 | NM_001097615 | EIF2B3 | NM_001261418 | SCRN1 | NM_001145514 |
| POLR2K | NM 005034 | EIF4G3 | NM 003760 | SDC2 | NM 002998 |
| POLR2L | NM_021128 | EMX2 | NM_001165924 | SEMA3E | NM_001178129 |
| POLR2M | NM 015532 | EN1 | NM 001426 | SEPN1 | NM 020451 |
| POLR3A | NM 007055 | ENHO | NM 198573 | SEPTIN9 | NM 001113491 |
| POLR3B | NM 001160708 | ENO2 | NM 001975 | SERF2 | NM 001199876 |
| POLR3B | NM 018082 | ENPP3 | NM 005021 | SERF2 | NM 001199877 |
| POLR3C | NM_006468 | ERCC6-PGBD3 | NM_001277059 | SERF2 | NM_001199878 |
| POLR3D | NM 001722 | ESRRG | NM 001134285 | SERPINB1 | NM 030666 |
| POLR3E | NM_001258034 | ESX1 | NM_153448 | SETD4 | NM_001007261 |
| POLR3F | NM 001282526 | EXOSC9 | NM 005033 | SETDB2 | NM 001160308 |
| POLR3G | NM 006467 | FAHD2B | NM 199336 | SETDB2 | NM 031915 |
| POLR3GL | NM_032305 | FAM118A | NM_017911 | SFMBT2 | NM_001018039 |
| POLR3H | NM 001282884 | FAM153A | NM 173663 | SFMBT2 | NM 001029880 |
| POLR3H | NM_001018052 | FAM222A | NM_032829 | SFTPC | NM_001172410 |
| POLR3K | NM 016310 | FAM58A | NM 001130997 | SGCB | NM 000232 |
| POLRMT | NM 005035 | FAM84A | NM 145175 | SH3TC1 | NM 018986 |
| RPL10 | NM_001256577 | FANCA | NM_001286167 | SHD | NM_020209 |
| RPL10A | NM 007104 | FASTKD2 | NM 014929 | SHE | NM 001010846 |
| RPL10L | NM_080746 | FBXL12 | NM_017703 | SHROOM3 | NM_020859 |
| RPL11 | NM 001199802 | FGF14 | NM 004115 | SLC12A6 | NM 001042497 |
| RPL12 | NM 000976 | FLCN | NM 144997 | SLC25A23 | NM 024103 |
| RPL13 | NM 001243130 | FLOT2 | NM 004475 | SLC2A8 | NM 014580 |
| RPL13 | NM 033251 | FLVCR2 | NM 001195283 | SLC35F3 | NM 173508 |
| RPL13A | NM_012423 | FNDC8 | NM_017559 | SLC4A2 | NM_001199692 |
| RPL14 | NM 001034996 | FRAT2 | NM 012083 | SLC7A13 | NM 138817 |
| RPL14 | NM_003973 | FSCN3 | NM_020369 | SLC9A1 | NM_003047 |
| RPL15 | NM 001253380 | FSD1 | NM 024333 | SLMO1 | NM 006553 |
| RPL15 | NM 001253384 | FUK | NM 145059 | SMARCAD1 | NM 001254949 |
| RPL15 | NM_001253382 | FUOM | NM_198472 | SMIM13 | NM_001135575 |
| RPL15 | NM 001253383 | FURIN | NM 001289824 | SOCS2 | NM 001270468 |
| RPL17 | NM_001199340 | FUT10 | NM_032664 | SOCS2 | NM_003877 |
| RPL17 | NM 001199342 | FUT6 | NM 000150 | SPEG | NM 001173476 |
| RPL17- | NM_001199355 | FXYD3 | NM_005971 | SPOCK3 | NM_016950 |
| C18orf32 | | | | | |
| RPL18 | NM 000979 | GABARAP | NM 007278 | SPTBN1 | NM 003128 |
| RPL18A | NM_000980 | GABBR1 | NM_021904 | SPTBN1 | NM_178313 |
| RPL19 | NM 000981 | GABRB3 | NM 001191321 | SRC | NM 198291 |
| RPL21 | NM_000982 | GABRR1 | NM_001256704 | SSX7 | NM_173358 |
| RPL22 | NM 000983 | GABRR1 | NM 001267582 | STAMBP | NM 006463 |
| RPL22L1 | NM 001099645 | GALK2 | NM 001001556 | STEAP2 | NM 001040666 |
| RPL23 | NM_000978 | GALK2 | NM_002044 | STEAP2 | NM_001244946 |
| RPL23A | NM 000984 | GATA2 | NM 001145662 | STK40 | NM 032017 |
| RPL24 | NM_000986 | GATA2 | NM_032638 | STRN3 | NM_001083893 |
| RPL26 | NM 000987 | GGN | NM 152657 | SULT2B1 | NM 177973 |
| RPL26L1 | NM 016093 | GHR | NM 001242406 | SUSD1 | NM 022486 |
| RPL27 | NM 000988 | GNAL | NM 001142339 | SUV39H1 | NM 003173 |
| RPL27A | NM 000990 | GNG13 | NM 016541 | SYAP1 | NM 032796 |
| RPL28 | NM_000991 | GNGT1 | NM_021955 | TAF1A | NM_001201536 |
| RPL29 | NM 000992 | GOLM1 | NM 016548 | TAPBPL | NM 018009 |
| RPL3 | NM_000967 | GPKOW | NM_015698 | TCF4 | NM_001243233 |
| RPL30 | NM 000989 | GPR119 | NM 178471 | TDO2 | NM 005651 |
| RPL31 | NM 001098577 | GPR87 | NM 023915 | TEAD4 | NM 003213 |
| RPL32 | NM_000994 | GPRASP2 | NM_001184876 | TEAD4 | NM_201443 |
| RPL32 | NM 001007074 | GPX6 | NM 182701 | TERF2IP | NM 018975 |
| RPL34 | NM_033625 | GRIP1 | NM_001178074 | TERT | NM_198253 |
| RPL34 | NM 000995 | GSTO2 | NM 001191014 | TGFBR2 | NM 003242 |
| RPL35 | NM 007209 | GUCY1A3 | NM 001130684 | TIFA | NM 052864 |
| RPL35A | NM 000996 | HAUS4 | NM 001166269 | TLE2 | NM 001144761 |
| RPL36 | NM 015414 | HBZ | NM 005332 | TLE3 | NM 020908 |
| RPL36 | NM_033643 | HES1 | NM_005524 | TMEM125 | NM_144626 |
| RPL36A- | NM 001199973 | HGD | NM 000187 | TMEM141 | NM 032928 |
| HNRNPH2 | | | | | |
| RPL36AL | NM_001001 | HHATL | NM_020707 | TMEM147 | NM_032635 |
| RPL37 | NM 000997 | HIST1H2BB | NM 021062 | TMEM164 | NM 017698 |
| RPL37A | NM_000998 | HMGB2 | NM_001130689 | TMEM170B | NM_001100829 |
| RPL38 | NM 000999 | HMMR | NM 012484 | TMEM201 | NM 001010866 |
| RPL39 | NM 001000 | HOOK1 | NM 015888 | TMEM223 | NM 001080501 |
| RPL39L | NM 052969 | HOXB6 | NM 018952 | TMEM25 | NM 032780 |
| RPL3L | NM 005061 | HRH2 | NM 022304 | TMEM5 | NM 014254 |
| RPL4 | NM_000968 | HRNR | NM_001009931 | TMEM53 | NM_024587 |
| RPL41 | NM 001035267 | HYPK | NM 016400 | TMEM56 | NM 001199679 |
| RPL5 | NM_000969 | IARS2 | NM_018060 | TMEM56 | NM_152487 |
| RPL6 | NM 000970 | IFI27L2 | NM 032036 | TMEM92 | NM 153229 |
| RPL7 | NM 000971 | IFNA7 | NM 021057 | TMPRSS5 | NM 030770 |
| RPL7A | NM_000972 | IFNAR1 | NM_000629 | TMX1 | NM_030755 |
| RPL7L1 | NM 198486 | IL15RA | NM 001243539 | TNIP2 | NM 001161527 |
| RPL8 | NM_000973 | IL18RAP | NM_003853 | TNIP2 | NM_024309 |
| RPL8 | NM 033301 | IL1A | NM 000575 | TNIP3 | NM 024873 |
| RPL9 | NM 001024921 | INA | NM 032727 | TNPO2 | NM 001136195 |
| RPL9 | NM 000661 | INO80 | NM 017553 | TNS3 | NM 022748 |
| RPLP0 | NM 001002 | INTS10 | NM 018142 | TPTE2 | NM 001271850 |
| RPLP1 | NM_213725 | INTS3 | NM_023015 | TREH | NM_007180 |
| RPLP2 | NM 001004 | IPO7 | NM 006391 | TRIM39 | NM 021253 |
| RPS10 | NM_001203245 | IQGAP2 | NM_001285460 | TRIM7 | NM_203293 |
| RPS10- | NM 001202470 | IRF5 | NM 032643 | TSHR | NM 000369 |
| NUDT3 | | | | | |
| RPS11 | NM 001015 | ISLR | NM 201526 | TSPAN5 | NM 005723 |
| RPS12 | NM_001016 | ISOC1 | NM_016048 | TSSK6 | NM_032037 |
| RPS13 | NM 001017 | ITLN1 | NM 017625 | TSTD3 | NM 001195131 |
| RPS14 | NM_005617 | JMJD7-PLA2G4B | NM_005090 | TTC37 | NM_014639 |
| RPS15 | NM 001018 | JUNB | NM 002229 | TULP2 | NM 003323 |
| RPS15A | NM 001019 | KCNF1 | NM 002236 | UBA5 | NM 024818 |
| RPS16 | NM_001020 | KCNH6 | NM_173092 | UBA5 | NM_198329 |
| RPS17 | NM 001021 | KCNQ2 | NM 004518 | UBASH3A | NM 018961 |
| RPS17L | NM_001199057 | KHNYN | NM_015299 | UBE2J1 | NM_016021 |
| RPS18 | NM 022551 | KIAA1456 | NM 001099677 | UGT1A9 | NM 021027 |
| RPS19 | NM 001022 | KIAA1456 | NM 020844 | UHRF1 | NM 001290050 |
| RPS19BP1 | NM 194326 | KIAA1462 | NM 020848 | UHRF1 | NM 001290051 |
| RPS2 | NM 002952 | KLHL2 | NM 007246 | URGCP-MRPS24 | NM 001204871 |
| RPS20 | NM_001146227 | KLK14 | NM_022046 | VCPIP1 | NM_025054 |
| RPS21 | NM 001024 | KLK9 | NM 012315 | VEPH1 | NM 024621 |
| RPS23 | NM_001025 | KLRC1 | NM_002259 | VGLL2 | NM_153453 |
| RPS24 | NM 033022 | KRAS | NM 004985 | VPS37A | NM 001145152 |
| RPS25 | NM 001028 | KRBA1 | NM 001290187 | VPS9D1 | NM 004913 |
| RPS26 | NM 001029 | KRT14 | NM 000526 | WARS2 | NM 015836 |
| RPS27 | NM_001030 | KRT17 | NM_000422 | WDR76 | NM_001167941 |
| RPS27A | NM 001177413 | KRT31 | NM 002277 | WRAP53 | NM 001143991 |
| RPS27A | NM_002954 | KRTAP20-2 | NM_181616 | XRCC6 | NM_001469 |
| RPS27A | NM 001135592 | KRTAP20-3 | NM 001128077 | XRN1 | NM 019001 |
| RPS27L | NM 015920 | KRTAP5-6 | NM 001012416 | YEATS4 | NM 006530 |
| RPS28 | NM_001031 | L3MBTL1 | NM_032107 | YTHDF3 | NM_001277817 |
| RPS29 | NM 001030001 | LARP1B | NM 018078 | YTHDF3 | NM 001277818 |
| RPS3 | NM_001005 | LDHB | NM_002300 | YY1AP1 | NM_001198902 |
| RPS3A | NM 001006 | LIFR | NM 002310 | YY1AP1 | NM 018253 |
| RPS4X | NM 001007 | LINGO4 | NM 001004432 | ZBTB10 | NM 001277145 |
| RPS4Y1 | NM 001008 | LMBRD2 | NM 001007527 | ZC3H6 | NM 198581 |
| RPS4Y2 | NM 001039567 | LMNA | NM 001257374 | ZFP41 | NM 001271156 |
| RPS5 | NM_001009 | LMO7 | NM_005358 | ZFP91 | NM_053023 |
| RPS6 | NM 001010 | LMX1B | NM 001174147 | ZFX | NM 001178086 |
| RPS6KA1 | NM_001006665 | LOC100131094 | NM_001242901 | ZMYM4 | NM_005095 |
| RPS6KA1 | NM 002953 | LOC100131303 | NM 001282442 | ZNF14 | NM 021030 |
| RPS6KA2 | NM 021135 | LRRC69 | NM 001129890 | ZNF182 | NM 006962 |
| RPS6KA2 | NM_001006932 | LRRFIP2 | NM_006309 | ZNF267 | NM_003414 |
| RPS6KA3 | NM 004586 | LSM4 | NM 012321 | ZNF30 | NM 194325 |
| RPS6KA4 | NM_003942 | LSMEM1 | NM_182597 | ZNF302 | NM_001289191 |
| RPS6KA5 | NM 004755 | LSP1 | NM 001013253 | ZNF471 | NM 020813 |
| RPS6KA6 | NM 014496 | LSP1 | NM 001289005 | ZNF486 | NM 052852 |
| RPS6KB1 | NM 003161 | LYG2 | NM 175735 | ZNF497 | NM 001207009 |
| RPS6KB2 | NM 003952 | LYVE1 | NM 006691 | ZNF560 | NM 152476 |
| RPS6KC1 | NM_001136138 | MACROD1 | NM_014067 | ZNF576 | NM_024327 |
| RPS6KL1 | NM 031464 | MACROD2 | NM 080676 | ZNF653 | NM 138783 |
| RPS7 | NM 001011 | MAGEA4 | NM 001011549 | ZNF710 | NM 198526 |
| RPS8 | NM 001012 | MAGEA4 | NM 001011550 | ZNF728 | NM 001267716 |
| RPS9 | NM 001013 | MAGEB17 | NM 001277307 | ZNF766 | NM 001010851 |
| RPSA | NM_002295 | MANEA | NM_024641 | ZNF81 | NM_007137 |
| RPSA | NM 001012321 | MAP3K7CL | NM 001286624 | ZNF827 | NM 178835 |
| | | MAP3K7CL | NM_001286634 | | |

TABLE 3A
FDR of the CRISPRa acute screening results. Gene targets are in ranked order.
Gene names, RefSeq IDs, and estimated FDR using negative control genes (see
Methods) for each of the top 1000 genes in the screening bioreps are listed.

| | | Acute no | Acute | Acute no | Acute |
|---|---|---|---|---|---|
| | | T cells | T cells | T cells | T cells |
| Gene | id | biorep 1 | biorep 1 | biorep 2 | biorep 2 |
| CYB5D2 | NM_001254755 | 0.00000 | 0.00000 | 0.00000 | 0.00322 |
| ISLR | NM_201526 | 0.00322 | 0.00322 | 0.00000 | 0.00000 |
| CD274 | NM_014143 | 0.00000 | 0.00322 | 0.00643 | 0.00000 |
| AHR | NM_001621 | 0.00643 | 0.00965 | 0.00322 | 0.00322 |
| WRAP53 | NM_001143991 | 0.02251 | 0.00322 | 0.00322 | 0.00000 |
| MCL1 | NM_021960 | 0.00000 | 0.00643 | 0.00000 | 0.02251 |
| MAGED2 | NM_014599 | 0.00000 | 0.00965 | 0.01608 | 0.00965 |
| KCNQ2 | NM_004518 | 0.00000 | 0.01608 | 0.00000 | 0.02251 |
| GNAL | NM_001142339 | 0.03215 | 0.00965 | 0.00000 | 0.00000 |
| MYBL1 | NM_001144755 | 0.00000 | 0.03859 | 0.00000 | 0.00322 |
| SCMH1 | NM_001172222 | 0.00322 | 0.00965 | 0.01929 | 0.00965 |
| C1R | NM_001733 | 0.00322 | 0.00965 | 0.01929 | 0.00965 |
| SDC2 | NM_002998 | 0.01286 | 0.00643 | 0.02251 | 0.00322 |
| ZBTB10 | NM_001277145 | 0.00000 | 0.00965 | 0.00000 | 0.04180 |
| CYP2S1 | NM_030622 | 0.01286 | 0.00965 | 0.01929 | 0.00965 |
| TBC1D9B | NM_015043 | 0.00643 | 0.01608 | 0.01608 | 0.01608 |
| SREK1IP1 | NM_173829 | 0.00000 | 0.01286 | 0.04180 | 0.00322 |
| JADE3 | NM_001077445 | 0.00322 | 0.02894 | 0.01608 | 0.00965 |
| KCTD18 | NM_152387 | 0.01286 | 0.00965 | 0.03215 | 0.00322 |
| LMNA | NM_001257374 | 0.01286 | 0.04502 | 0.00000 | 0.00322 |
| JMJD1C | NM_001282948 | 0.00965 | 0.01608 | 0.00322 | 0.03215 |
| EGR3 | NM_004430 | 0.02572 | 0.01286 | 0.00643 | 0.01608 |
| RPS7 | NM_001011 | 0.00965 | 0.00322 | 0.02572 | 0.02894 |
| STAG2 | NM_001042749 | 0.01286 | 0.02251 | 0.01929 | 0.01286 |
| JUNB | NM_002229 | 0.00000 | 0.00000 | 0.05788 | 0.00965 |
| HIST3H2BB | NM_175055 | 0.01286 | 0.02251 | 0.01286 | 0.01929 |
| LGALS9C | NM_001040078 | 0.00000 | 0.00643 | 0.01929 | 0.05466 |
| C12orf73 | NM_001135570 | 0.03215 | 0.03859 | 0.00000 | 0.00965 |
| GRIP1 | NM_001178074 | 0.00000 | 0.00322 | 0.06752 | 0.01608 |
| COMMD4 | NM_017828 | 0.06431 | 0.00965 | 0.00643 | 0.00643 |
| TBX20 | NM_001077653 | 0.02251 | 0.00965 | 0.01929 | 0.03537 |
| CENPE | NM_001813 | 0.02251 | 0.02894 | 0.02251 | 0.01608 |
| MEIS2 | NM_172315 | 0.00965 | 0.01286 | 0.07395 | 0.00965 |
| HIST1H2BI | NM_003525 | 0.00965 | 0.02572 | 0.03537 | 0.04180 |
| TLE2 | NM_001144761 | 0.02251 | 0.09003 | 0.00000 | 0.00000 |
| DHX33 | NM_001199699 | 0.05788 | 0.03859 | 0.01608 | 0.00000 |
| RABEPK | NM_001174153 | 0.05466 | 0.06431 | 0.00000 | 0.00000 |
| RREB1 | NM_001003699 | 0.00643 | 0.09003 | 0.00322 | 0.02251 |
| COX14 | NM_001257134 | 0.00000 | 0.00322 | 0.06752 | 0.05466 |
| CGREF1 | NM_001166241 | 0.00965 | 0.09003 | 0.00643 | 0.02251 |
| MFSD12 | NM_174983 | 0.04180 | 0.02251 | 0.02251 | 0.04180 |
| TSPY1 | NM_001197242 | 0.08360 | 0.04180 | 0.00322 | 0.00322 |
| CXCL5 | NM_002994 | 0.00000 | 0.00322 | 0.05466 | 0.07395 |
| BCAR1 | NM_001170719 | 0.06431 | 0.01286 | 0.02251 | 0.03215 |
| IL34 | NM_001172772 | 0.00965 | 0.01286 | 0.00643 | 0.10932 |
| SYNCRIP | NM_001159673 | 0.07717 | 0.00322 | 0.00000 | 0.05788 |
| ANXA8 | NM_001271702 | 0.03215 | 0.10611 | 0.00000 | 0.00000 |
| DSC1 | NM_004948 | 0.00000 | 0.04502 | 0.06752 | 0.02572 |
| CDK19 | NM_015076 | 0.00000 | 0.00643 | 0.10611 | 0.02572 |
| SLC35G4 | NM_001282300 | 0.00643 | 0.00965 | 0.07074 | 0.05466 |
| LAMC3 | NM_006059 | 0.04180 | 0.04180 | 0.00643 | 0.05145 |
| FLNA | NM_001110556 | 0.05145 | 0.04180 | 0.01608 | 0.03537 |
| KIF21A | NM_001173463 | 0.04180 | 0.08039 | 0.02251 | 0.00000 |
| KCNK6 | NM_004823 | 0.05788 | 0.00643 | 0.03215 | 0.04823 |
| TCF23 | NM_175769 | 0.00000 | 0.00965 | 0.13505 | 0.00322 |
| CDO1 | NM_001801 | 0.00000 | 0.00322 | 0.03215 | 0.11254 |
| PRKACB | NM_002731 | 0.06431 | 0.08039 | 0.00643 | 0.00000 |
| STARD13 | NM_178006 | 0.11576 | 0.02572 | 0.00643 | 0.00322 |
| TMEM19 | NM_018279 | 0.03859 | 0.03859 | 0.01929 | 0.05466 |
| EPHA1 | NM_005232 | 0.00965 | 0.00965 | 0.04180 | 0.09646 |
| TMEM87B | NM_032824 | 0.03859 | 0.09646 | 0.01286 | 0.00965 |
| FLCN | NM_144997 | 0.06109 | 0.09646 | 0.00000 | 0.00000 |
| RABGGTA | NM_182836 | 0.06109 | 0.08360 | 0.00643 | 0.00965 |
| PPIC | NM_000943 | 0.00322 | 0.00965 | 0.07074 | 0.07717 |
| TRIM58 | NM_015431 | 0.00965 | 0.09003 | 0.02251 | 0.04180 |
| ZNF583 | NM_152478 | 0.14148 | 0.02251 | 0.00000 | 0.00000 |
| CSTB | NM_000100 | 0.08039 | 0.05466 | 0.01608 | 0.01608 |
| DCST2 | NM_144622 | 0.03859 | 0.00965 | 0.10611 | 0.01286 |
| BCL2L1 | NM_001191 | 0.04180 | 0.03859 | 0.08682 | 0.00322 |
| REG4 | NM_001159352 | 0.08360 | 0.00965 | 0.00322 | 0.07395 |
| RNF166 | NM_001171816 | 0.03859 | 0.07074 | 0.00965 | 0.05466 |
| RNLS | NM_018363 | 0.02251 | 0.00965 | 0.11576 | 0.02572 |
| ZBTB7B | NM_001252406 | 0.06431 | 0.07395 | 0.01608 | 0.02251 |
| RASA2 | NM_006506 | 0.06431 | 0.02251 | 0.01608 | 0.07717 |
| ST8SIA5 | NM_013305 | 0.05466 | 0.08039 | 0.04180 | 0.00643 |
| ACYP1 | NM_001107 | 0.01286 | 0.00965 | 0.12862 | 0.03537 |
| DUS4L | NM_181581 | 0.00965 | 0.04502 | 0.04180 | 0.09003 |
| NXNL1 | NM_138454 | 0.07074 | 0.08360 | 0.00965 | 0.02251 |
| PCNA | NM_182649 | 0.00965 | 0.01286 | 0.06109 | 0.10289 |
| TCTE3 | NM_174910 | 0.05466 | 0.09646 | 0.00643 | 0.03215 |
| C7orf76 | NM_001201451 | 0.08360 | 0.08360 | 0.00322 | 0.02251 |
| SYCE1 | NM_001143764 | 0.00322 | 0.04823 | 0.09003 | 0.05466 |
| PPP1R1B | NM_032192 | 0.00965 | 0.00965 | 0.12219 | 0.05466 |
| CSTF1 | NM_001033522 | 0.06431 | 0.08039 | 0.04180 | 0.00965 |
| RARRES3 | NM_004585 | 0.04180 | 0.09325 | 0.05788 | 0.00322 |
| SNX20 | NM_001144972 | 0.04180 | 0.11576 | 0.03215 | 0.00965 |
| NLRP2 | NM_001174081 | 0.08360 | 0.02572 | 0.01608 | 0.07395 |
| SLC1A1 | NM_004170 | 0.03859 | 0.14791 | 0.00000 | 0.01286 |
| AMZ1 | NM_001284355 | 0.04180 | 0.08682 | 0.03215 | 0.04180 |
| NACC1 | NM_052876 | 0.09968 | 0.08360 | 0.00322 | 0.01608 |
| SRBD1 | NM_018079 | 0.05466 | 0.06752 | 0.01286 | 0.07074 |
| METRN | NM_024042 | 0.00000 | 0.00322 | 0.17685 | 0.02572 |
| MYL4 | NM_001002841 | 0.05145 | 0.09003 | 0.01608 | 0.05145 |
| ERMP1 | NM_024896 | 0.03215 | 0.04502 | 0.11576 | 0.01608 |
| TBC1D2 | NM_001267572 | 0.00643 | 0.06109 | 0.08682 | 0.05466 |
| STK4 | NM_006282 | 0.02572 | 0.02572 | 0.08039 | 0.07717 |
| AKR1C1 | NM_001353 | 0.02251 | 0.00965 | 0.15434 | 0.02572 |
| PRICKLE1 | NM_153026 | 0.01286 | 0.02894 | 0.11897 | 0.05466 |
| NAV3 | NM_014903 | 0.00322 | 0.03859 | 0.07395 | 0.09968 |
| GNPDA2 | NM_138335 | 0.14148 | 0.00965 | 0.05788 | 0.00643 |
| ZNF281 | NM_001281294 | 0.00965 | 0.00965 | 0.14791 | 0.05145 |
| MYO15A | NM_016239 | 0.14791 | 0.00965 | 0.00643 | 0.05466 |
| FOXC1 | NM_001453 | 0.04180 | 0.06431 | 0.08682 | 0.02572 |
| GSTCD | NM_024751 | 0.08360 | 0.05145 | 0.03215 | 0.05466 |
| CATSPERB | NM_024764 | 0.05466 | 0.06431 | 0.02894 | 0.07395 |
| RAB27A | NM_183234 | 0.06752 | 0.09968 | 0.01286 | 0.04180 |
| ACVRL1 | NM_001077401 | 0.01608 | 0.02572 | 0.01286 | 0.17042 |
| LIPT1 | NM_145198 | 0.08682 | 0.03859 | 0.08682 | 0.01286 |
| MAGEA11 | NM_005366 | 0.05145 | 0.08039 | 0.07074 | 0.02251 |
| TYK2 | NM_003331 | 0.03215 | 0.07395 | 0.02572 | 0.09325 |
| HES1 | NM_005524 | 0.00000 | 0.00965 | 0.11576 | 0.10289 |
| TAAR2 | NM_014626 | 0.03859 | 0.02251 | 0.07717 | 0.09003 |
| YY1AP1 | NM_001198903 | 0.06109 | 0.08039 | 0.08682 | 0.00322 |
| TRIM39 | NM_021253 | 0.10289 | 0.12540 | 0.00000 | 0.00322 |
| C1QTNF9B- | NM_001014442 | 0.02251 | 0.06752 | 0.06752 | 0.07395 |
| AS1 | | | | | |
| UBA3 | NM_003968 | 0.03859 | 0.02572 | 0.12862 | 0.04180 |
| CPXCR1 | NM_033048 | 0.02251 | 0.03859 | 0.01608 | 0.16077 |
| C20orf197 | NM_173644 | 0.08360 | 0.02572 | 0.02572 | 0.10289 |
| ARNT | NM_001668 | 0.11576 | 0.10932 | 0.01608 | 0.00000 |
| PRKAG1 | NM_002733 | 0.08360 | 0.13826 | 0.01608 | 0.00322 |
| NOTCH2 | NM_024408 | 0.09968 | 0.11576 | 0.01608 | 0.00965 |
| ARMCX5 | NM_001168480 | 0.14148 | 0.09325 | 0.00643 | 0.00322 |
| SOCS2 | NM_001270468 | 0.00965 | 0.02572 | 0.11897 | 0.09003 |
| PHF21A | NM_016621 | 0.14148 | 0.06431 | 0.02894 | 0.00965 |
| SMIM13 | NM_001135575 | 0.12219 | 0.06752 | 0.01608 | 0.04180 |
| MRPL23 | NM_021134 | 0.03215 | 0.05788 | 0.10611 | 0.05466 |
| TRAF3 | NM_001199427 | 0.03859 | 0.09003 | 0.01286 | 0.11254 |
| SMIM12 | NM_138428 | 0.07395 | 0.12540 | 0.04180 | 0.01286 |
| HPSE2 | NM_001166244 | 0.06752 | 0.09003 | 0.01929 | 0.07717 |
| GOS2 | NM_015714 | 0.06109 | 0.01608 | 0.07074 | 0.10932 |
| OTOP1 | NM_177998 | 0.08360 | 0.08360 | 0.01608 | 0.07717 |
| OR4C11 | NM_001004700 | 0.16399 | 0.06431 | 0.02251 | 0.00965 |
| ZNF385B | NM_001113398 | 0.10611 | 0.10289 | 0.01608 | 0.03537 |
| PREPL | NM_006036 | 0.08360 | 0.13826 | 0.02251 | 0.01608 |
| UBE2NL | NM_001012989 | 0.03859 | 0.05466 | 0.01929 | 0.15113 |
| BCL6 | NM_001706 | 0.04180 | 0.03215 | 0.02251 | 0.16720 |
| HIST1H4J | NM_021968 | 0.04180 | 0.02572 | 0.18006 | 0.01608 |
| PROSC | NM_007198 | 0.12540 | 0.11254 | 0.01608 | 0.01608 |
| COL9A3 | NM_001853 | 0.12540 | 0.10289 | 0.00643 | 0.03537 |
| ZNF763 | NM_001012753 | 0.13183 | 0.09325 | 0.00643 | 0.04180 |
| DEFB127 | NM_139074 | 0.11576 | 0.15756 | 0.00000 | 0.00000 |
| ALS2CL | NM_001190707 | 0.06431 | 0.08039 | 0.02894 | 0.09968 |
| RPS3A | NM_001006 | 0.05145 | 0.05466 | 0.01608 | 0.15434 |
| CST4 | NM_001899 | 0.00000 | 0.00322 | 0.25723 | 0.01608 |
| DPP6 | NM_001039350 | 0.06752 | 0.03215 | 0.00643 | 0.17042 |
| CELF2 | NM_001083591 | 0.03859 | 0.12540 | 0.02251 | 0.09003 |
| KRT31 | NM_002277 | 0.11576 | 0.15434 | 0.00322 | 0.00322 |
| ATXNIL | NM_001137675 | 0.16077 | 0.08360 | 0.01929 | 0.01608 |
| CUZD1 | NM_022034 | 0.13183 | 0.08360 | 0.02251 | 0.04180 |
| SFMBT2 | NM_001018039 | 0.10611 | 0.16720 | 0.00322 | 0.00322 |
| FBXO9 | NM_033481 | 0.09968 | 0.09968 | 0.01286 | 0.06752 |
| RPS27A | NM_001135592 | 0.10932 | 0.15113 | 0.00643 | 0.01608 |
| ARL4A | NM_001195396 | 0.09968 | 0.11576 | 0.04180 | 0.02572 |
| TAF1 | NM_001286074 | 0.08360 | 0.03859 | 0.01608 | 0.14791 |
| SRSF9 | NM_003769 | 0.12540 | 0.16077 | 0.00000 | 0.00000 |
| GPBAR1 | NM_170699 | 0.14148 | 0.00965 | 0.08682 | 0.04823 |
| COLEC11 | NM_001255988 | 0.14469 | 0.03859 | 0.06752 | 0.03537 |
| TRIM64C | NM_001206631 | 0.06752 | 0.00965 | 0.01608 | 0.19614 |
| CXorf57 | NM_018015 | 0.07717 | 0.09325 | 0.01608 | 0.10289 |
| RECK | NM_021111 | 0.05145 | 0.21865 | 0.01608 | 0.00322 |
| HGD | NM_000187 | 0.17685 | 0.08360 | 0.01929 | 0.00965 |
| PRDM8 | NM_020226 | 0.15434 | 0.10932 | 0.00322 | 0.02251 |
| SMIM19 | NM_138436 | 0.09646 | 0.00322 | 0.11576 | 0.07395 |
| STK25 | NM_001271978 | 0.00000 | 0.02572 | 0.18650 | 0.07717 |
| KLHL25 | NM_022480 | 0.19936 | 0.00965 | 0.06752 | 0.01608 |
| CYP1A2 | NM_000761 | 0.12540 | 0.09003 | 0.00643 | 0.07074 |
| TMEM116 | NM_001193453 | 0.16399 | 0.12219 | 0.00322 | 0.00322 |
| DEFA3 | NM_005217 | 0.08682 | 0.06431 | 0.08682 | 0.05466 |
| ACOT6 | NM_001037162 | 0.13826 | 0.15434 | 0.00000 | 0.00000 |
| PPFIBP2 | NM_003621 | 0.03215 | 0.02894 | 0.03215 | 0.19936 |
| COMMD1 | NM_152516 | 0.04180 | 0.03215 | 0.14148 | 0.07717 |
| AVEN | NM_020371 | 0.01286 | 0.08360 | 0.14469 | 0.05466 |
| MBD3L3 | NM_001164425 | 0.01286 | 0.02572 | 0.08682 | 0.17042 |
| MGAT5 | NM_002410 | 0.07395 | 0.08039 | 0.06752 | 0.07395 |
| ZNF675 | NM_138330 | 0.14469 | 0.07074 | 0.03215 | 0.04823 |
| BBS5 | NM_152384 | 0.03215 | 0.04180 | 0.07074 | 0.15113 |
| LIN54 | NM_001115007 | 0.16399 | 0.08360 | 0.01608 | 0.03537 |
| F8 | NM_000132 | 0.14148 | 0.08039 | 0.04180 | 0.03537 |
| C18orf21 | NM_001201476 | 0.00000 | 0.00965 | 0.25723 | 0.03215 |
| CDC20 | NM_001255 | 0.15113 | 0.14791 | 0.00000 | 0.00000 |
| PRAF2 | NM_007213 | 0.10289 | 0.09325 | 0.02251 | 0.08039 |
| ZFP62 | NM_152283 | 0.07395 | 0.13826 | 0.03215 | 0.05466 |
| TSEN34 | NM_001282333 | 0.16077 | 0.08360 | 0.01608 | 0.04180 |
| FRAT2 | NM_012083 | 0.01286 | 0.00643 | 0.08682 | 0.19614 |
| FAM78A | NM_033387 | 0.14148 | 0.08360 | 0.02251 | 0.05466 |
| P2RX5 | NM_001204519 | 0.16077 | 0.13826 | 0.00000 | 0.00322 |
| GPHN | NM_020806 | 0.03859 | 0.06752 | 0.00643 | 0.18971 |
| LSMEM2 | NM_153215 | 0.08682 | 0.03215 | 0.09325 | 0.09325 |
| TCEAL2 | NM_080390 | 0.06109 | 0.01608 | 0.06752 | 0.16077 |
| PFKFB2 | NM_006212 | 0.12219 | 0.08360 | 0.02251 | 0.07717 |
| GPT2 | NM_001142466 | 0.10289 | 0.15113 | 0.01286 | 0.04180 |
| SPATA31A6 | NM_001145196 | 0.00965 | 0.21543 | 0.01608 | 0.06752 |
| COMTD1 | NM_144589 | 0.03215 | 0.08360 | 0.02251 | 0.17042 |
| MAP3K13 | NM_001242317 | 0.07395 | 0.08360 | 0.09968 | 0.05466 |
| EIF1AD | NM_001242484 | 0.05466 | 0.06431 | 0.08360 | 0.10932 |
| JADE3 | NM_014735 | 0.19936 | 0.03859 | 0.03215 | 0.04180 |
| TMX1 | NM_030755 | 0.00000 | 0.00322 | 0.08360 | 0.22508 |
| MYO1B | NM_001161819 | 0.03215 | 0.03859 | 0.11897 | 0.12219 |
| FBXO42 | NM_018994 | 0.16399 | 0.11576 | 0.01929 | 0.01608 |
| TBX21 | NM_013351 | 0.10289 | 0.01286 | 0.05788 | 0.14148 |
| LINC01272 | NM_001278655 | 0.00965 | 0.02251 | 0.16077 | 0.12540 |
| TMEM256 | NM_152766 | 0.11897 | 0.11576 | 0.06752 | 0.01608 |
| HTR6 | NM_000871 | 0.09968 | 0.09646 | 0.08682 | 0.03537 |
| ZNF667 | NM_022103 | 0.11576 | 0.07395 | 0.06752 | 0.06109 |
| PCDHA12 | NM_018903 | 0.00965 | 0.00965 | 0.17042 | 0.13183 |
| LNX2 | NM_153371 | 0.09003 | 0.04502 | 0.01929 | 0.16720 |
| KRTAP10-2 | NM_198693 | 0.04180 | 0.08360 | 0.01608 | 0.18006 |
| SLC16A11 | NM_153357 | 0.17685 | 0.13826 | 0.00322 | 0.00322 |
| C18orf8 | NM_001276342 | 0.23151 | 0.05466 | 0.02251 | 0.01608 |
| FKBP2 | NM_004470 | 0.12540 | 0.11576 | 0.01608 | 0.06752 |
| TES | NM_152829 | 0.12219 | 0.15434 | 0.00643 | 0.04180 |
| POC5 | NM_001099271 | 0.10289 | 0.10932 | 0.08682 | 0.02572 |
| ANO3 | NM_031418 | 0.09968 | 0.03215 | 0.04180 | 0.15113 |
| ARSF | NM_001201538 | 0.20900 | 0.09003 | 0.01929 | 0.00965 |
| ALKBH6 | NM_032878 | 0.06752 | 0.05466 | 0.20257 | 0.00322 |
| SHROOM2 | NM_001649 | 0.01286 | 0.01286 | 0.27010 | 0.03215 |
| PHF1 | NM_002636 | 0.06752 | 0.16077 | 0.06431 | 0.03537 |
| TCEAL4 | NM_024863 | 0.19614 | 0.08682 | 0.02251 | 0.02251 |
| PABPC4L | NM_001114734 | 0.03215 | 0.02251 | 0.09646 | 0.17685 |
| SNX27 | NM_030918 | 0.11576 | 0.03215 | 0.01608 | 0.16720 |
| HAPLN4 | NM_023002 | 0.24759 | 0.01929 | 0.03215 | 0.03215 |
| KCNQ5 | NM_001160130 | 0.09968 | 0.09003 | 0.07074 | 0.07395 |
| INTS10 | NM_018142 | 0.00000 | 0.00965 | 0.07395 | 0.25080 |
| TMEM234 | NM_019118 | 0.14148 | 0.07395 | 0.04180 | 0.07717 |
| RORC | NM_005060 | 0.11576 | 0.14469 | 0.04180 | 0.03215 |
| MSH6 | NM_001281494 | 0.01286 | 0.00965 | 0.23473 | 0.08039 |
| IMP3 | NM_018285 | 0.08360 | 0.04502 | 0.01608 | 0.19293 |
| OR7A5 | NM_017506 | 0.00000 | 0.00965 | 0.07395 | 0.25402 |
| SLC35C1 | NM_018389 | 0.06752 | 0.08360 | 0.10932 | 0.07717 |
| ATAD3B | NM_031921 | 0.04180 | 0.07395 | 0.08360 | 0.13826 |
| AMPD1 | NM_000036 | 0.10289 | 0.10289 | 0.08039 | 0.05145 |
| UBE2J1 | NM_016021 | 0.08360 | 0.24116 | 0.00643 | 0.00965 |
| GUCY1A3 | NM_001130684 | 0.01608 | 0.03859 | 0.15756 | 0.12862 |
| SLFNL1 | NM_001168247 | 0.15756 | 0.11576 | 0.01286 | 0.05466 |
| FAM115A | NM_001206938 | 0.01286 | 0.00965 | 0.08682 | 0.23151 |
| OR9Q1 | NM_001005212 | 0.14148 | 0.16077 | 0.02251 | 0.01608 |
| C1orf27 | NM_001164246 | 0.03859 | 0.05466 | 0.12862 | 0.12219 |
| SLC4A8 | NM_001258403 | 0.00965 | 0.03859 | 0.12540 | 0.17042 |
| ATF7IP | NM_018179 | 0.05145 | 0.15756 | 0.02572 | 0.11254 |
| MAP2K2 | NM_030662 | 0.12540 | 0.00965 | 0.18971 | 0.02251 |
| DNASE1L1 | NM_006730 | 0.09968 | 0.11897 | 0.08682 | 0.04180 |
| LYVE1 | NM_006691 | 0.20900 | 0.12540 | 0.00643 | 0.00965 |
| MTHFD2 | NM_006636 | 0.08360 | 0.18328 | 0.01608 | 0.06752 |
| PDF | NM_022341 | 0.01608 | 0.00965 | 0.01929 | 0.30547 |
| KDELC1 | NM_024089 | 0.16077 | 0.09003 | 0.09646 | 0.00322 |
| MBD3L2 | NM_144614 | 0.00322 | 0.00965 | 0.10611 | 0.23151 |
| EPHA6 | NM_001278300 | 0.06431 | 0.11576 | 0.12862 | 0.04180 |
| BCL7B | NM_001707 | 0.00643 | 0.08039 | 0.01929 | 0.24437 |
| SLFNL1 | NM_144990 | 0.04180 | 0.11897 | 0.02251 | 0.16720 |
| ZNF664- | NM_001204299 | 0.14148 | 0.10289 | 0.09325 | 0.01608 |
| FAM101A | | | | | |
| MASP2 | NM_006610 | 0.16399 | 0.09003 | 0.03215 | 0.06752 |
| UNKL | NM_001276414 | 0.11576 | 0.08360 | 0.01608 | 0.13826 |
| SEPP1 | NM_005410 | 0.10289 | 0.10289 | 0.09325 | 0.05466 |
| CHERP | NM_006387 | 0.12540 | 0.09003 | 0.02572 | 0.11254 |
| ITM2C | NM_030926 | 0.00000 | 0.00322 | 0.08360 | 0.26688 |
| MARVELD3 | NM_052858 | 0.16077 | 0.07395 | 0.08360 | 0.03537 |
| SH2D1B | NM_053282 | 0.09003 | 0.02572 | 0.09003 | 0.14791 |
| ISLR2 | NM_001130137 | 0.04180 | 0.02572 | 0.03215 | 0.25402 |
| OPRK1 | NM_000912 | 0.06109 | 0.12540 | 0.02894 | 0.13826 |
| PIP5K1C | NM_012398 | 0.11576 | 0.06431 | 0.02251 | 0.15113 |
| UBAC2 | NM_001144072 | 0.16077 | 0.07395 | 0.07395 | 0.04823 |
| BCAT1 | NM_001178093 | 0.16077 | 0.15113 | 0.04180 | 0.00322 |
| BAHD1 | NM_014952 | 0.16077 | 0.15756 | 0.02251 | 0.01608 |
| MTA2 | NM_004739 | 0.19614 | 0.10611 | 0.02572 | 0.02894 |
| ASL | NM_001024943 | 0.20900 | 0.09325 | 0.05466 | 0.00322 |
| CERKL | NM_001160277 | 0.16077 | 0.10932 | 0.04180 | 0.04823 |
| MFNG | NM_001166343 | 0.04180 | 0.00965 | 0.08682 | 0.22186 |
| NGFR | NM_002507 | 0.10289 | 0.08039 | 0.01608 | 0.16077 |
| ZKSCAN3 | NM_024493 | 0.03215 | 0.02572 | 0.20900 | 0.09325 |
| CDKNIA | NM_001220778 | 0.03215 | 0.02572 | 0.05788 | 0.24437 |
| HYAL4 | NM_012269 | 0.05145 | 0.02894 | 0.02251 | 0.25723 |
| OLFM1 | NM_001282612 | 0.27653 | 0.02572 | 0.01286 | 0.04823 |
| CDK16 | NM_033018 | 0.03859 | 0.10611 | 0.20257 | 0.01608 |
| CACNB2 | NM_201596 | 0.15434 | 0.09003 | 0.01608 | 0.10289 |
| ZSCAN26 | NM_152736 | 0.00965 | 0.04180 | 0.02251 | 0.28939 |
| DEFB130 | NM_001037804 | 0.08360 | 0.00965 | 0.22186 | 0.05145 |
| CORO6 | NM_032854 | 0.23473 | 0.12540 | 0.00322 | 0.00322 |
| CETN2 | NM_004344 | 0.15113 | 0.16077 | 0.01608 | 0.04180 |
| CXCL1 | NM_001511 | 0.11576 | 0.24437 | 0.00643 | 0.00322 |
| SATB2 | NM_015265 | 0.13183 | 0.19614 | 0.01608 | 0.02572 |
| CORO1B | NM_020441 | 0.18971 | 0.16077 | 0.00322 | 0.01608 |
| OLIG1 | NM_138983 | 0.16077 | 0.19614 | 0.00000 | 0.01286 |
| SMPDL3B | NM_001009568 | 0.03215 | 0.04180 | 0.01608 | 0.27974 |
| KCNQ3 | NM_004519 | 0.04180 | 0.03859 | 0.09325 | 0.19614 |
| TMEM261 | NM_033428 | 0.04180 | 0.05466 | 0.19614 | 0.07717 |
| PANK1 | NM_138316 | 0.05145 | 0.04823 | 0.02251 | 0.24759 |
| SMC4 | NM_001288753 | 0.03859 | 0.06109 | 0.06431 | 0.20579 |
| PLEKHF1 | NM_024310 | 0.04180 | 0.00965 | 0.08682 | 0.23473 |
| ZAN | NM_003386 | 0.23151 | 0.13183 | 0.00643 | 0.00643 |
| ZNF214 | NM_013249 | 0.16077 | 0.16077 | 0.01608 | 0.03859 |
| TBL1X | NM_001139467 | 0.04180 | 0.05466 | 0.16720 | 0.11254 |
| CFC1 | NM_032545 | 0.16077 | 0.18650 | 0.01608 | 0.01286 |
| STAT3 | NM_213662 | 0.08360 | 0.10611 | 0.01608 | 0.17042 |
| PWWP2B | NM_138499 | 0.10289 | 0.22508 | 0.00643 | 0.04180 |
| TAL2 | NM_005421 | 0.04180 | 0.10611 | 0.17363 | 0.05466 |
| NLRC4 | NM_021209 | 0.06109 | 0.19936 | 0.07395 | 0.04180 |
| LPPR5 | NM_001037317 | 0.11576 | 0.23151 | 0.01608 | 0.01608 |
| DNAH14 | NM_144989 | 0.04180 | 0.05466 | 0.15113 | 0.13183 |
| ST6GALNAC2 | NM_006456 | 0.02894 | 0.21543 | 0.11897 | 0.01608 |
| ANXA2 | NM_001136015 | 0.04180 | 0.00322 | 0.14148 | 0.19293 |
| C11orf70 | NM_001195005 | 0.10289 | 0.02251 | 0.08682 | 0.16720 |
| RING1 | NM_002931 | 0.21865 | 0.12219 | 0.00322 | 0.03537 |
| NOV | NM_002514 | 0.17363 | 0.09646 | 0.06752 | 0.04180 |
| ZMYM4 | NM_005095 | 0.02251 | 0.03859 | 0.09646 | 0.22186 |
| FUT6 | NM_000150 | 0.14148 | 0.24116 | 0.00000 | 0.00000 |
| C15orf41 | NM_001130010 | 0.11576 | 0.09003 | 0.09646 | 0.08039 |
| ARPC4 | NM_001024960 | 0.14148 | 0.01286 | 0.09003 | 0.13826 |
| PQBP1 | NM_001167990 | 0.11576 | 0.12540 | 0.10611 | 0.03537 |
| URB1 | NM_014825 | 0.06109 | 0.09003 | 0.20579 | 0.02572 |
| LHX9 | NM_020204 | 0.00322 | 0.06431 | 0.08360 | 0.23151 |
| CASKIN2 | NM_001142643 | 0.07717 | 0.09325 | 0.08360 | 0.13183 |
| HSF1 | NM_005526 | 0.08360 | 0.19936 | 0.03215 | 0.07074 |
| CBL | NM_005188 | 0.11576 | 0.21543 | 0.02251 | 0.03215 |
| SPTBN1 | NM_003128 | 0.08360 | 0.00322 | 0.23473 | 0.07074 |
| DPP6 | NM_001290253 | 0.16399 | 0.06431 | 0.08682 | 0.07717 |
| CTRL | NM_001907 | 0.09003 | 0.15113 | 0.05788 | 0.09325 |
| SMAD4 | NM_005359 | 0.24759 | 0.10611 | 0.00000 | 0.03859 |
| COQ2 | NM_015697 | 0.05145 | 0.03859 | 0.20257 | 0.09968 |
| BIN3 | NM_018688 | 0.11576 | 0.27010 | 0.00322 | 0.00322 |
| LMOD1 | NM_012134 | 0.18650 | 0.15113 | 0.01929 | 0.03537 |
| H2AFY2 | NM_018649 | 0.14469 | 0.11576 | 0.09325 | 0.04180 |
| FAM175A | NM_139076 | 0.09003 | 0.07395 | 0.07074 | 0.16077 |
| ANKRD66 | NM_001162435 | 0.11576 | 0.03859 | 0.05788 | 0.18650 |
| HEY2 | NM_012259 | 0.18328 | 0.14469 | 0.01929 | 0.05145 |
| SNCA | NM_000345 | 0.11897 | 0.08360 | 0.10932 | 0.09003 |
| RNF121 | NM_018320 | 0.17042 | 0.21543 | 0.01286 | 0.00322 |
| SLC45A3 | NM_033102 | 0.19936 | 0.13826 | 0.02251 | 0.04180 |
| MNAT1 | NM_002431 | 0.10289 | 0.02572 | 0.03215 | 0.24116 |
| HNRNPCL1 | NM_001013631 | 0.16077 | 0.00322 | 0.15113 | 0.09003 |
| UQCRHL | NM_001089591 | 0.07074 | 0.09325 | 0.03215 | 0.20900 |
| C9orf50 | NM_199350 | 0.00643 | 0.01608 | 0.12540 | 0.25723 |
| AMMECR1L | NM_031445 | 0.08360 | 0.23151 | 0.01286 | 0.07717 |
| PLEKHG5 | NM_001042665 | 0.03859 | 0.02894 | 0.17685 | 0.16077 |
| OR5R1 | NM_001004744 | 0.04180 | 0.13826 | 0.15113 | 0.07395 |
| TRHDE | NM_013381 | 0.18328 | 0.21543 | 0.00322 | 0.00322 |
| CHST5 | NM_024533 | 0.17042 | 0.08360 | 0.08682 | 0.06752 |
| TTC38 | NM_017931 | 0.16077 | 0.17363 | 0.01929 | 0.05466 |
| ZNF750 | NM_024702 | 0.21543 | 0.12219 | 0.06109 | 0.00965 |
| IMMP1L | NM_144981 | 0.14148 | 0.15113 | 0.01608 | 0.09968 |
| MAPKAPK3 | NM_004635 | 0.06109 | 0.18006 | 0.09003 | 0.07717 |
| COL4A5 | NM_000495 | 0.08360 | 0.09325 | 0.16077 | 0.07395 |
| NMU | NM_006681 | 0.11576 | 0.24759 | 0.01608 | 0.03215 |
| SERPINB12 | NM_080474 | 0.19293 | 0.16077 | 0.02251 | 0.03537 |
| CHMP1A | NM_001083314 | 0.00000 | 0.00322 | 0.13826 | 0.27010 |
| USP5 | NM_001098536 | 0.20900 | 0.13505 | 0.04180 | 0.02572 |
| TDRD3 | NM_001146070 | 0.11576 | 0.21543 | 0.01286 | 0.06752 |
| CAPG | NM_001256139 | 0.08039 | 0.13826 | 0.17042 | 0.02251 |
| RARRES2 | NM_002889 | 0.19936 | 0.17363 | 0.02251 | 0.01608 |
| ECI2 | NM_006117 | 0.10289 | 0.21543 | 0.08360 | 0.00965 |
| ELAC2 | NM_001165962 | 0.26045 | 0.09003 | 0.01929 | 0.04180 |
| COL11A1 | NM_001190709 | 0.14469 | 0.09003 | 0.16720 | 0.01286 |
| CASC1 | NM_001204102 | 0.16077 | 0.13183 | 0.08682 | 0.03537 |
| SOS1 | NM_005633 | 0.11576 | 0.09003 | 0.04180 | 0.16720 |
| PNLIPRP1 | NM_006229 | 0.00000 | 0.00322 | 0.12862 | 0.28296 |
| GABARAPL2 | NM_007285 | 0.18650 | 0.09003 | 0.12219 | 0.01608 |
| KIAA1217 | NM_001098500 | 0.23794 | 0.09968 | 0.03537 | 0.04180 |
| XRCC6BP1 | NM_033276 | 0.04180 | 0.02572 | 0.27331 | 0.07395 |
| DMPK | NM_001081563 | 0.29582 | 0.02572 | 0.05788 | 0.03537 |
| AMER3 | NM_001105193 | 0.24759 | 0.16077 | 0.00322 | 0.00643 |
| TMEM95 | NM_198154 | 0.19614 | 0.17042 | 0.01608 | 0.03537 |
| KCNIP4 | NM_001035004 | 0.06109 | 0.18328 | 0.07074 | 0.10289 |
| NXPE3 | NM_001134456 | 0.27653 | 0.08360 | 0.00643 | 0.05466 |
| CLN6 | NM_017882 | 0.15434 | 0.09003 | 0.01286 | 0.16399 |
| MAGEE1 | NM_020932 | 0.00965 | 0.04180 | 0.11897 | 0.25080 |
| BCAS1 | NM_003657 | 0.03859 | 0.08682 | 0.25723 | 0.04180 |
| IFNA7 | NM_021057 | 0.00000 | 0.00322 | 0.15113 | 0.27010 |
| ZNF395 | NM_018660 | 0.09968 | 0.00322 | 0.27010 | 0.05145 |
| PTGES3 | NM_006601 | 0.09968 | 0.15756 | 0.12862 | 0.04180 |
| SEPTIN1 | NM_052838 | 0.09003 | 0.25080 | 0.07074 | 0.01608 |
| FAM195B | NM_001288799 | 0.01608 | 0.08039 | 0.02572 | 0.30547 |
| MAZ | NM_002383 | 0.11576 | 0.09325 | 0.08039 | 0.13826 |
| ALS2 | NM_020919 | 0.02251 | 0.00965 | 0.30868 | 0.08682 |
| SEBOX | NM_001080837 | 0.02251 | 0.02572 | 0.36977 | 0.00965 |
| GON4L | NM_001282856 | 0.20900 | 0.08360 | 0.08360 | 0.05466 |
| CXorf23 | NM_198279 | 0.16077 | 0.17042 | 0.08682 | 0.01286 |
| CNTNAP3B | NM_001201380 | 0.12219 | 0.04502 | 0.06752 | 0.19614 |
| HIST1H4F | NM_003540 | 0.19936 | 0.21543 | 0.00000 | 0.01608 |
| NKG7 | NM_005601 | 0.03215 | 0.03859 | 0.28617 | 0.07395 |
| EXOC5 | NM_006544 | 0.03215 | 0.09003 | 0.14791 | 0.16077 |
| PACRG | NM_001080378 | 0.16720 | 0.10289 | 0.08360 | 0.07717 |
| SIPA1L1 | NM_001284245 | 0.14469 | 0.15434 | 0.08360 | 0.04823 |
| NUS1 | NM_138459 | 0.16077 | 0.09003 | 0.04180 | 0.13826 |
| SETD1A | NM_014712 | 0.14791 | 0.23473 | 0.04180 | 0.00965 |
| NACAD | NM_001146334 | 0.32476 | 0.09646 | 0.00322 | 0.00965 |
| OSGIN2 | NM_004337 | 0.11576 | 0.12540 | 0.11576 | 0.07717 |
| PAICS | NM_006452 | 0.20579 | 0.02572 | 0.01286 | 0.18971 |
| TOR1A | NM_000113 | 0.19936 | 0.12219 | 0.01929 | 0.09325 |
| TLE3 | NM_020908 | 0.18650 | 0.09325 | 0.04180 | 0.11254 |
| C16orf93 | NM_001014979 | 0.06752 | 0.00965 | 0.11254 | 0.24759 |
| KLF7 | NM_001270942 | 0.03859 | 0.00965 | 0.18006 | 0.20900 |
| BTBD7 | NM_001289133 | 0.09003 | 0.09003 | 0.07074 | 0.18650 |
| FBXW12 | NM_207102 | 0.17363 | 0.16720 | 0.08682 | 0.00965 |
| CEACAM1 | NM_001024912 | 0.09968 | 0.07395 | 0.07074 | 0.19293 |
| PTRH2 | NM_016077 | 0.10289 | 0.07395 | 0.23473 | 0.02572 |
| KLK7 | NM_005046 | 0.25080 | 0.15434 | 0.02251 | 0.00965 |
| MAML1 | NM_014757 | 0.07395 | 0.02251 | 0.11897 | 0.22186 |
| ARID1B | NM_020732 | 0.11576 | 0.28939 | 0.02251 | 0.00965 |
| SPP2 | NM_006944 | 0.14469 | 0.12862 | 0.12540 | 0.04180 |
| PHACTR4 | NM_023923 | 0.00965 | 0.00322 | 0.17042 | 0.25723 |
| ASB2 | NM_001202429 | 0.14148 | 0.08039 | 0.18650 | 0.03215 |
| HIST1H2BH | NM_003524 | 0.06109 | 0.08360 | 0.26367 | 0.03215 |
| KRTAP26-1 | NM_203405 | 0.02251 | 0.03859 | 0.09003 | 0.28939 |
| CAMKK2 | NM_172226 | 0.11576 | 0.09003 | 0.22830 | 0.00965 |
| NGLY1 | NM_001145294 | 0.11576 | 0.15113 | 0.07395 | 0.10289 |
| MLPH | NM_001281473 | 0.00965 | 0.02572 | 0.30547 | 0.10289 |
| HDLBP | NM_005336 | 0.10289 | 0.13826 | 0.16720 | 0.03537 |
| SEC22A | NM_012430 | 0.27010 | 0.09968 | 0.03215 | 0.04180 |
| MRPS7 | NM_015971 | 0.23151 | 0.14469 | 0.04502 | 0.02251 |
| CCDC116 | NM_152612 | 0.02251 | 0.02894 | 0.02251 | 0.36977 |
| TBC1D4 | NM_014832 | 0.16077 | 0.15756 | 0.07395 | 0.05466 |
| NKX2-1 | NM_001079668 | 0.08682 | 0.08360 | 0.00643 | 0.27010 |
| ANKRD37 | NM_181726 | 0.04823 | 0.07074 | 0.09646 | 0.23151 |
| OR2Z1 | NM_001004699 | 0.13505 | 0.11576 | 0.09325 | 0.10289 |
| MAPK1 | NM_002745 | 0.06431 | 0.06431 | 0.21865 | 0.09968 |
| PRMT8 | NM_001256536 | 0.14148 | 0.20900 | 0.04502 | 0.05466 |
| PDE1B | NM_001165975 | 0.16077 | 0.16720 | 0.02251 | 0.09968 |
| CCNC | NM_005190 | 0.14148 | 0.17363 | 0.09325 | 0.04180 |
| PM20D2 | NM_001010853 | 0.00965 | 0.08360 | 0.01929 | 0.33762 |
| ATP1A1 | NM_001160233 | 0.04180 | 0.02894 | 0.30868 | 0.07074 |
| HMCES | NM_020187 | 0.23794 | 0.16077 | 0.03215 | 0.01929 |
| C8orf76 | NM_032847 | 0.31190 | 0.13826 | 0.00000 | 0.00000 |
| TBC1D10C | NM_198517 | 0.10289 | 0.09646 | 0.21543 | 0.03537 |
| FAM65C | NM_080829 | 0.01286 | 0.09003 | 0.12862 | 0.22186 |
| PCOLCE2 | NM_013363 | 0.04180 | 0.10289 | 0.14148 | 0.16720 |
| TMEM213 | NM_001085429 | 0.16720 | 0.23151 | 0.01929 | 0.03537 |
| LRRC29 | NM_001004055 | 0.24759 | 0.18971 | 0.00322 | 0.01608 |
| EML2 | NM_001193269 | 0.05466 | 0.08360 | 0.14791 | 0.17042 |
| MUC22 | NM_001198815 | 0.02251 | 0.09003 | 0.02251 | 0.32154 |
| NTF3 | NM_001102654 | 0.19614 | 0.09968 | 0.02251 | 0.13826 |
| PCGF5 | NM_032373 | 0.11897 | 0.09003 | 0.08039 | 0.17042 |
| DNAJA4 | NM_001130183 | 0.16720 | 0.20257 | 0.08682 | 0.00322 |
| CTSK | NM_000396 | 0.09968 | 0.09003 | 0.08682 | 0.18328 |
| PAX6 | NM_001258463 | 0.18650 | 0.12540 | 0.04180 | 0.10611 |
| ACADVL | NM_001270447 | 0.23473 | 0.15434 | 0.02251 | 0.04823 |
| LOC100131303 | NM_001282442 | 0.00000 | 0.00322 | 0.08682 | 0.36977 |
| FUT4 | NM_002033 | 0.03859 | 0.03859 | 0.09003 | 0.29260 |
| FBLIM1 | NM_017556 | 0.03859 | 0.08360 | 0.02572 | 0.31190 |
| MED31 | NM_016060 | 0.04180 | 0.08360 | 0.17042 | 0.16720 |
| BTN2A1 | NM_001197234 | 0.07074 | 0.08360 | 0.10289 | 0.20579 |
| HERC1 | NM_003922 | 0.03215 | 0.07395 | 0.08682 | 0.27010 |
| SLAIN2 | NM_020846 | 0.14469 | 0.15756 | 0.11897 | 0.04180 |
| ANP32E | NM_001136479 | 0.02572 | 0.02251 | 0.16077 | 0.25402 |
| PNPLA6 | NM_001166111 | 0.14148 | 0.10932 | 0.17685 | 0.03537 |
| CARD11 | NM_032415 | 0.10289 | 0.08360 | 0.21222 | 0.06431 |
| C1orf35 | NM_024319 | 0.08039 | 0.09325 | 0.08360 | 0.20900 |
| MYO1C | NM_033375 | 0.00000 | 0.01286 | 0.17685 | 0.27653 |
| CPNE9 | NM_153635 | 0.05466 | 0.00965 | 0.06752 | 0.33441 |
| ZNF354C | NM_014594 | 0.06752 | 0.03859 | 0.27331 | 0.08682 |
| SLITRK3 | NM_014926 | 0.17042 | 0.28939 | 0.00322 | 0.00322 |
| GNAI2 | NM_002070 | 0.10289 | 0.21865 | 0.11576 | 0.02894 |
| HOXB13 | NM_006361 | 0.06109 | 0.02572 | 0.30225 | 0.07717 |
| PRDM13 | NM_021620 | 0.04180 | 0.02251 | 0.18006 | 0.22186 |
| C16orf13 | NM_032366 | 0.10289 | 0.15434 | 0.02251 | 0.18650 |
| NFKBIE | NM_004556 | 0.01608 | 0.42122 | 0.00000 | 0.03215 |
| TMEM201 | NM_001010866 | 0.11576 | 0.16077 | 0.10611 | 0.08682 |
| FGF22 | NM_020637 | 0.03859 | 0.11897 | 0.21865 | 0.09325 |
| VNIR5 | NM_173858 | 0.16077 | 0.03215 | 0.10932 | 0.16720 |
| XRCC2 | NM_005431 | 0.09003 | 0.06431 | 0.22830 | 0.08682 |
| GBP4 | NM_052941 | 0.01608 | 0.02572 | 0.25723 | 0.17042 |
| VCX | NM_013452 | 0.14148 | 0.12862 | 0.17685 | 0.02251 |
| NTF4 | NM_006179 | 0.11576 | 0.21543 | 0.08682 | 0.05466 |
| SUMO2 | NM_006937 | 0.09003 | 0.21543 | 0.08682 | 0.08039 |
| ZFP28 | NM_020828 | 0.00000 | 0.00322 | 0.38907 | 0.08039 |
| BIRC5 | NM_001168 | 0.10289 | 0.00965 | 0.17042 | 0.18971 |
| TMUB2 | NM_177441 | 0.14148 | 0.16077 | 0.07074 | 0.09968 |
| N4BP2L2 | NM_033111 | 0.06431 | 0.10611 | 0.07074 | 0.23151 |
| OLFM1 | NM_006334 | 0.23151 | 0.21865 | 0.01608 | 0.00965 |
| FBLIM1 | NM_001024216 | 0.09003 | 0.11576 | 0.04180 | 0.22830 |
| DEGS1 | NM_003676 | 0.12540 | 0.07395 | 0.20257 | 0.07395 |
| GJB6 | NM_006783 | 0.05466 | 0.07395 | 0.24437 | 0.10289 |
| OR12D2 | NM_013936 | 0.12540 | 0.22508 | 0.01286 | 0.11254 |
| PBRM1 | NM_018313 | 0.20579 | 0.21543 | 0.00000 | 0.05466 |
| FSD1 | NM_024333 | 0.26367 | 0.19936 | 0.01286 | 0.00000 |
| GH2 | NM_022556 | 0.16077 | 0.25080 | 0.02251 | 0.04180 |
| CDKN2A | NM_000077 | 0.25080 | 0.21543 | 0.00643 | 0.00322 |
| TSTD1 | NM_001113207 | 0.11576 | 0.04180 | 0.21865 | 0.09968 |
| ACSL6 | NM_001205251 | 0.16077 | 0.18328 | 0.02251 | 0.10932 |
| HPR | NM_020995 | 0.02251 | 0.09325 | 0.17685 | 0.18328 |
| DEDD | NM_001039712 | 0.29260 | 0.15756 | 0.01286 | 0.01608 |
| FARS2 | NM_006567 | 0.16399 | 0.11576 | 0.15756 | 0.04180 |
| CLEC4E | NM_014358 | 0.20579 | 0.21543 | 0.01608 | 0.04180 |
| SMARCAD1 | NM_001254949 | 0.01608 | 0.05466 | 0.11897 | 0.28939 |
| NOTCH1 | NM_017617 | 0.05466 | 0.19936 | 0.01929 | 0.20579 |
| TMEM47 | NM_031442 | 0.15434 | 0.08360 | 0.09325 | 0.14791 |
| ATG3 | NM_022488 | 0.26367 | 0.21543 | 0.00000 | 0,00000 |
| ZFX | NM_001178086 | 0.00000 | 0.00322 | 0.07074 | 0.40514 |
| STAM | NM_003473 | 0.13505 | 0.20257 | 0.03215 | 0.10932 |
| C6orf223 | NM_153246 | 0.06109 | 0.19293 | 0.01929 | 0.20579 |
| ARHGAP29 | NM_004815 | 0.08360 | 0.02251 | 0.06431 | 0.30868 |
| PHF8 | NM_001184898 | 0.08360 | 0.17042 | 0.09646 | 0.13183 |
| SLC15A1 | NM_005073 | 0.08360 | 0.16077 | 0.01608 | 0.22186 |
| BAMBI | NM_012342 | 0.11576 | 0.08360 | 0.11576 | 0.16720 |
| NOP14 | NM_003703 | 0.02251 | 0.03215 | 0.15756 | 0.27010 |
| PHPT1 | NM_001287343 | 0.04180 | 0.24116 | 0.18650 | 0.01608 |
| SAMD3 | NM_001258275 | 0.00643 | 0.01286 | 0.14148 | 0.32476 |
| LILRB4 | NM_001278427 | 0.03537 | 0.08360 | 0.19614 | 0.17042 |
| LSM7 | NM_016199 | 0.08039 | 0.20900 | 0.03537 | 0.16077 |
| SLC2A6 | NM_001145099 | 0.14469 | 0.13826 | 0.04180 | 0.16077 |
| IQCJ-SCHIP1 | NM_001197113 | 0.02251 | 0.02251 | 0.16077 | 0.27974 |
| PHLPP2 | NM_001289003 | 0.09968 | 0.11897 | 0.08682 | 0.18006 |
| CELA2B | NM_015849 | 0.00000 | 0.00322 | 0.06752 | 0.41479 |
| MAFA | NM_201589 | 0.29582 | 0.09968 | 0.01608 | 0.07395 |
| RRM2 | NM_001165931 | 0.13505 | 0.00965 | 0.22830 | 0.11254 |
| LOC81691 | NM_001199053 | 0.14148 | 0.09968 | 0.17685 | 0.06752 |
| C1orf228 | NM_001145636 | 0.16077 | 0.14791 | 0.05466 | 0.12540 |
| C4orf26 | NM_001257072 | 0.05145 | 0.24116 | 0.18650 | 0.00965 |
| HOXC13 | NM_017410 | 0.11576 | 0.09325 | 0.10932 | 0.17042 |
| HIF3A | NM_152794 | 0.01608 | 0.03215 | 0.17042 | 0.27010 |
| CLEC12A | NM_201623 | 0.07717 | 0.08039 | 0.17042 | 0.16077 |
| RBFOX1 | NM_001142333 | 0.19614 | 0.21543 | 0.04180 | 0.03537 |
| MXRA8 | NM_001282583 | 0.23794 | 0.08360 | 0.09968 | 0.06752 |
| GATA1 | NM_002049 | 0.14148 | 0.30868 | 0.02251 | 0.01608 |
| WNT5B | NM_030775 | 0.10289 | 0.16077 | 0.08682 | 0.13826 |
| PDZD4 | NM_032512 | 0.05788 | 0.01286 | 0.25080 | 0.16720 |
| MAST3 | NM_015016 | 0.07717 | 0.03859 | 0.18650 | 0.18650 |
| SLC25A37 | NM_016612 | 0.08360 | 0.13826 | 0.12862 | 0.13826 |
| PRPF38A | NM_032864 | 0.05466 | 0.04823 | 0.11576 | 0.27331 |
| KIAA1549L | NM_012194 | 0.16077 | 0.23794 | 0.02251 | 0.07074 |
| SLC7A5 | NM_003486 | 0.27010 | 0.19614 | 0.02251 | 0.00322 |
| CUEDC2 | NM_024040 | 0.27010 | 0.13826 | 0.00322 | 0.08039 |
| HOXA4 | NM_002141 | 0.18006 | 0.26688 | 0.01929 | 0.02572 |
| YOD1 | NM_018566 | 0.16077 | 0.17042 | 0.07074 | 0.09325 |
| ANAPC11 | NM_001002245 | 0.00643 | 0.00965 | 0.14148 | 0.33762 |
| HOOK2 | NM_001100176 | 0.18328 | 0.19293 | 0.07074 | 0.04823 |
| MED14 | NM_004229 | 0.13183 | 0.06431 | 0.26367 | 0.03537 |
| ALDOA | NM_001243177 | 0.36977 | 0.06752 | 0.02251 | 0.03537 |
| BCKDHB | NM_000056 | 0.18650 | 0.08360 | 0.04180 | 0.18650 |
| TRPM3 | NM_001007471 | 0.14469 | 0.13826 | 0.02251 | 0.19293 |
| SLCO1B3 | NM_019844 | 0.17042 | 0.20900 | 0.06752 | 0.05466 |
| CCNA1 | NM_001111047 | 0.15113 | 0.11576 | 0.06752 | 0.16720 |
| LRP4 | NM_002334 | 0.14148 | 0.21543 | 0.09325 | 0.05145 |
| HAND2 | NM_021973 | 0.10289 | 0.12540 | 0.06752 | 0.20579 |
| C17orf100 | NM_001105520 | 0.34405 | 0.08360 | 0.01929 | 0.05466 |
| OR5K4 | NM_001005517 | 0.10289 | 0.06431 | 0.06431 | 0.27010 |
| CHCHD4 | NM_001098502 | 0.05145 | 0.06431 | 0.12540 | 0.26367 |
| PRR35 | NM_145270 | 0.16077 | 0.18971 | 0.12862 | 0.02572 |
| ODF3L2 | NM_182577 | 0.10289 | 0.11576 | 0.07717 | 0.20900 |
| UTP6 | NM_018428 | 0.11576 | 0.23151 | 0.08682 | 0.07074 |
| MAGEC3 | NM_138702 | 0.14148 | 0.10611 | 0.09646 | 0.16077 |
| ESRRB | NM_004452 | 0.18006 | 0.21543 | 0.03215 | 0.07717 |
| RNASEH2A | NM_006397 | 0.29260 | 0.10611 | 0.09646 | 0.00965 |
| SBSN | NM_001166034 | 0.19936 | 0.24116 | 0.01286 | 0.05466 |
| PUM2 | NM_001282791 | 0.08360 | 0.17363 | 0.05466 | 0.19614 |
| SMAD1 | NM_005900 | 0.17042 | 0.21543 | 0.09003 | 0.03215 |
| CDK16 | NM_006201 | 0.16720 | 0.21543 | 0.04180 | 0.08360 |
| EPPK1 | NM_031308 | 0.17363 | 0.16077 | 0.09325 | 0.08039 |
| MID1 | NM_001193277 | 0.08360 | 0.10289 | 0.02572 | 0.29582 |
| BTF3 | NM_001037637 | 0.16077 | 0.25080 | 0.01608 | 0.08360 |
| STEAP2 | NM_001040666 | 0.04180 | 0.16077 | 0.12862 | 0.18006 |
| PSMA6 | NM_001282234 | 0.13183 | 0.09968 | 0.09325 | 0.18650 |
| EMP1 | NM_001423 | 0.16077 | 0.12540 | 0.01929 | 0.20579 |
| SLC18A1 | NM_003053 | 0.12540 | 0.02894 | 0.18650 | 0.17042 |
| KRTAP21-2 | NM_181617 | 0.19936 | 0.13826 | 0.09325 | 0.08039 |
| HRASLS5 | NM_054108 | 0.14148 | 0.14791 | 0.06431 | 0.15756 |
| CCDC22 | NM_014008 | 0.18006 | 0.24437 | 0.01929 | 0.06752 |
| HSBP1 | NM_001537 | 0.12540 | 0.25402 | 0.04180 | 0.09325 |
| IFT81 | NM_014055 | 0.17685 | 0.32154 | 0.00322 | 0.01286 |
| NLGN4Y | NM_001206850 | 0.14148 | 0.05466 | 0.12862 | 0.18971 |
| RNF8 | NM_003958 | 0.09003 | 0.08360 | 0.11897 | 0.22186 |
| APOLD1 | NM_030817 | 0.15113 | 0.18971 | 0.12219 | 0.05145 |
| NOSTRIN | NM_001039724 | 0.06431 | 0.19936 | 0.01929 | 0.23151 |
| IRX3 | NM_024336 | 0.14148 | 0.16077 | 0.10932 | 0.10289 |
| OR2AG2 | NM_001004490 | 0.09003 | 0.09003 | 0.12862 | 0.20900 |
| SDHB | NM_003000 | 0.15756 | 0.27974 | 0.06431 | 0.01608 |
| MORN3 | NM_173855 | 0.03859 | 0.08360 | 0.12540 | 0.27010 |
| KRTAP13-2 | NM_181621 | 0.06752 | 0.18650 | 0.09325 | 0.17042 |
| CTDSP1 | NM_001206878 | 0.03859 | 0.23151 | 0.05788 | 0.18971 |
| HIST1H4G | NM_003547 | 0.09968 | 0.02572 | 0.11897 | 0.27331 |
| BTBD2 | NM_017797 | 0.11576 | 0.25080 | 0.08682 | 0.06431 |
| ATP8B2 | NM_020452 | 0.05466 | 0.09968 | 0.18006 | 0.18328 |
| OR5AN1 | NM_001004729 | 0.00322 | 0.01608 | 0.39228 | 0.10932 |
| AASDHPPT | NM_015423 | 0.20900 | 0.07717 | 0.09646 | 0.13826 |
| TMEM11 | NM_003876 | 0.17685 | 0.13183 | 0.18650 | 0.02572 |
| TDO2 | NM_005651 | 0.17685 | 0.16720 | 0.09646 | 0.08039 |
| NOS1AP | NM_014697 | 0.29260 | 0.16720 | 0.02572 | 0.03537 |
| KCNA3 | NM_002232 | 0.13183 | 0.23794 | 0.11576 | 0.03859 |
| KHDC1L | NM_001126063 | 0.05466 | 0.06752 | 0.34727 | 0.05466 |
| GABPA | NM_001197297 | 0.16077 | 0.35048 | 0.00322 | 0.00965 |
| URGCP- | NM_001204871 | 0.19614 | 0.32476 | 0.00000 | 0.00322 |
| MRPS24 | | | | | |
| NFAT5 | NM_001113178 | 0.15434 | 0.21543 | 0.04180 | 0.11254 |
| LOC100129520 | NM_001195272 | 0.17685 | 0.11576 | 0.02251 | 0.20900 |
| MINPP1 | NM_001178118 | 0.15434 | 0.32476 | 0.01929 | 0.02572 |
| MB21D2 | NM_178496 | 0.03859 | 0.02572 | 0.22830 | 0.23151 |
| MTUS1 | NM_001001931 | 0.27010 | 0.13183 | 0.02251 | 0.10289 |
| EFCAB3 | NM_001144933 | 0.09003 | 0.13183 | 0.27331 | 0.03537 |
| SLC19A1 | NM_001205207 | 0.23151 | 0.19293 | 0.08360 | 0.02251 |
| SECISBP2 | NM_001282690 | 0.14469 | 0.26367 | 0.00965 | 0.11254 |
| LIN7A | NM_004664 | 0.09968 | 0.09003 | 0.16077 | 0.18006 |
| TBL2 | NM_012453 | 0.06752 | 0.02572 | 0.16720 | 0.27010 |
| USP53 | NM_019050 | 0.08360 | 0.11576 | 0.13505 | 0.19614 |
| OSR1 | NM_145260 | 0.08360 | 0.08360 | 0.05788 | 0.30547 |
| DYNLT3 | NM_006520 | 0.01608 | 0.02251 | 0.22186 | 0.27010 |
| BLOC1S1 | NM_001487 | 0.13505 | 0.09646 | 0.11897 | 0.18006 |
| KLF11 | NM_001177716 | 0.13183 | 0.04180 | 0.09003 | 0.27010 |
| HIST1H3A | NM_003529 | 0.11576 | 0.20900 | 0.08682 | 0.12219 |
| APLP1 | NM_005166 | 0.09003 | 0.08360 | 0.22830 | 0.13183 |
| CXCL13 | NM_006419 | 0.23794 | 0.27010 | 0.01608 | 0.00965 |
| BCL2A1 | NM_001114735 | 0.00000 | 0.00322 | 0.11897 | 0.41158 |
| SLC35F2 | NM_017515 | 0.13505 | 0.11576 | 0.15756 | 0.12540 |
| C1orf21 | NM_030806 | 0.31511 | 0.21543 | 0.00322 | 0.00000 |
| S100A13 | NM_001024210 | 0.18328 | 0.02572 | 0.06752 | 0.25723 |
| LAMTOR2 | NM_001145264 | 0.06752 | 0.13183 | 0.08360 | 0.25402 |
| FGR | NM_001042747 | 0.14148 | 0.10289 | 0.08682 | 0.20579 |
| CCDC30 | NM_001080850 | 0.09003 | 0.17363 | 0.06431 | 0.20900 |
| PRELP | NM_002725 | 0.16077 | 0.34084 | 0.02572 | 0.00965 |
| PIGS | NM_033198 | 0.14791 | 0.07395 | 0.09325 | 0.22186 |
| DGCR6L | NM_033257 | 0.14148 | 0.12540 | 0.22830 | 0.04180 |
| IP6K3 | NM_054111 | 0.10289 | 0.33119 | 0.05466 | 0.04823 |
| PTPRB | NM_001109754 | 0.04823 | 0.09968 | 0.10611 | 0.28296 |
| RAB4A | NM_001271998 | 0.27010 | 0.08682 | 0.04180 | 0.13826 |
| SECISBP2 | NM_024077 | 0.23151 | 0.11576 | 0.01929 | 0.17042 |
| ZNF491 | NM_152356 | 0.05788 | 0.07717 | 0.25723 | 0.14469 |
| CHST8 | NM_001127895 | 0.25080 | 0.15434 | 0.05788 | 0.07395 |
| MGRN1 | NM_001142291 | 0.17685 | 0.15113 | 0.08039 | 0.13183 |
| LPAR6 | NM_001162497 | 0.14469 | 0.10289 | 0.20579 | 0.08682 |
| SLC12A1 | NM_001184832 | 0.14148 | 0.21543 | 0.01608 | 0.16720 |
| PSME2 | NM_002818 | 0.17685 | 0.22508 | 0.08360 | 0.05466 |
| NAT16 | NM_198571 | 0.08360 | 0.27010 | 0.08682 | 0.09968 |
| AKAP3 | NM_001278309 | 0.05466 | 0.13183 | 0.12540 | 0.23151 |
| ECHS1 | NM_004092 | 0.11576 | 0.10289 | 0.11897 | 0.20579 |
| STAG3 | NM_012447 | 0.19936 | 0.23151 | 0.02251 | 0.09003 |
| RANBP10 | NM_020850 | 0.06752 | 0.11576 | 0.08360 | 0.27653 |
| ZNF182 | NM_001178099 | 0.31511 | 0.01286 | 0.00000 | 0.21543 |
| FAM64A | NM_001195228 | 0.15756 | 0.13505 | 0.15756 | 0.09325 |
| PTPN14 | NM_005401 | 0.05145 | 0.14148 | 0.12862 | 0.22186 |
| ARRDC2 | NM_015683 | 0.01608 | 0.09003 | 0.14791 | 0.28939 |
| AZIN1 | NM_015878 | 0.16399 | 0.11576 | 0.18650 | 0.07717 |
| ASZ1 | NM_130768 | 0.12540 | 0.03859 | 0.18006 | 0.19936 |
| RFC4 | NM_181573 | 0.01286 | 0.00965 | 0.41801 | 0.10289 |
| CD247 | NM_198053 | 0.10289 | 0.10932 | 0.08360 | 0.24759 |
| SLC25A35 | NM_201520 | 0.02251 | 0.01286 | 0.20257 | 0.30547 |
| MAP3K3 | NM_203351 | 0.19293 | 0.30868 | 0.00965 | 0.03215 |
| KCND1 | NM_004979 | 0.13183 | 0.13183 | 0.04180 | 0.24116 |
| GPR174 | NM_032553 | 0.04180 | 0.09325 | 0.17042 | 0.24116 |
| RIIAD1 | NM_001144956 | 0.32476 | 0.21543 | 0.00322 | 0.00322 |
| NFS1 | NM_001198989 | 0.10289 | 0.15434 | 0.08682 | 0.20257 |
| NLRC4 | NM_001199139 | 0.11576 | 0.08682 | 0.20579 | 0.13826 |
| CNP | NM_033133 | 0.05466 | 0.06431 | 0.22186 | 0.20579 |
| EMP3 | NM_001425 | 0.32797 | 0.08039 | 0.09646 | 0.04180 |
| NXF1 | NM_001081491 | 0.06752 | 0.02572 | 0.08039 | 0.37299 |
| GLIPR2 | NM_001287013 | 0.02251 | 0.05466 | 0.06109 | 0.40836 |
| SEMA4B | NM_020210 | 0.20900 | 0.28296 | 0.01608 | 0.04180 |
| SLC39A13 | NM_001128225 | 0.16720 | 0.12540 | 0.08682 | 0.17042 |
| ETFB | NM_001985 | 0.21543 | 0.31190 | 0.00643 | 0.01608 |
| PRSS1 | NM_002769 | 0.16077 | 0.03859 | 0.33762 | 0.01286 |
| LHX3 | NM_014564 | 0.04180 | 0.02572 | 0.28617 | 0.19614 |
| PRPF38B | NM_018061 | 0.11576 | 0.09325 | 0.11897 | 0.22186 |
| LMF1 | NM_022773 | 0.00000 | 0.00322 | 0.27010 | 0.27653 |
| ANKRD24 | NM_133475 | 0.14469 | 0.04502 | 0.31833 | 0.04180 |
| ARHGAP11A | NM_014783 | 0.19614 | 0.24116 | 0.03215 | 0.08039 |
| STX1B | NM_052874 | 0.20900 | 0.09003 | 0.24437 | 0.00965 |
| PKD2L2 | NM_001258449 | 0.31511 | 0.14791 | 0.01608 | 0.07395 |
| R3HDM1 | NM_001282799 | 0.00643 | 0.00965 | 0.26688 | 0.27010 |
| ARHGEF10 | NM_014629 | 0.12540 | 0.21543 | 0.17042 | 0.04180 |
| PQBP1 | NM_001167989 | 0.25080 | 0.30225 | 0.00000 | 0.00000 |
| FADD | NM_003824 | 0.17042 | 0.09646 | 0.10611 | 0.18006 |
| RPL5 | NM_000969 | 0.08039 | 0.08039 | 0.30225 | 0.09325 |
| CYB5RL | NM_001031672 | 0.28617 | 0.21543 | 0.02251 | 0.03215 |
| DOK7 | NM_173660 | 0.25080 | 0.22186 | 0.06752 | 0.01608 |
| MAFF | NM_001161573 | 0.18328 | 0.31833 | 0.01608 | 0.04180 |
| BTN3A2 | NM_001197249 | 0.23794 | 0.21543 | 0.09003 | 0.01608 |
| SBDS | NM_016038 | 0.40514 | 0.01286 | 0.01608 | 0.12540 |
| FRMPD3 | NM_032428 | 0.01608 | 0.01608 | 0.29582 | 0.23151 |
| MORF4L1 | NM_206839 | 0.19936 | 0.26367 | 0.01608 | 0.08039 |
| GPRASP1 | NM_001099411 | 0.52090 | 0.03859 | 0.00000 | 0.00000 |
| ANKRD35 | NM_001280799 | 0.03215 | 0.02894 | 0.33762 | 0.16077 |
| ZMYM2 | NM_001190965 | 0.16077 | 0.11576 | 0.11576 | 0.17042 |
| CISH | NM_013324 | 0.16720 | 0.13826 | 0.08682 | 0.17042 |
| RSL1D1 | NM_015659 | 0.05145 | 0.08360 | 0.10611 | 0.32154 |
| EXOC6 | NM_019053 | 0.19936 | 0.09003 | 0.07395 | 0.19936 |
| OR4F29 | NM_001005221 | 0.16077 | 0.29260 | 0.01929 | 0.09003 |
| CBX1 | NM_001127228 | 0.36334 | 0.15434 | 0.02572 | 0.01929 |
| ACAA1 | NM_001607 | 0.30868 | 0.21543 | 0.02251 | 0.01608 |
| CUL5 | NM_003478 | 0.03215 | 0.03859 | 0.27010 | 0.22186 |
| MLYCD | NM_012213 | 0.15113 | 0.35691 | 0.01929 | 0.03537 |
| CEP68 | NM_015147 | 0.06109 | 0.07717 | 0.12862 | 0.29582 |
| S100A4 | NM_019554 | 0.27010 | 0.19936 | 0.01929 | 0.07395 |
| SLCO1B7 | NM_001009562 | 0.16077 | 0.24116 | 0.08682 | 0.07717 |
| RPS27A | NM_001177413 | 0.16720 | 0.11897 | 0.04823 | 0.23151 |
| SIGLEC5 | NM_003830 | 0.16399 | 0.09003 | 0.12862 | 0.18328 |
| CLUH | NM_015229 | 0.07395 | 0.02894 | 0.39228 | 0.07074 |
| KIAA1033 | NM_015275 | 0.16077 | 0.17363 | 0.12862 | 0.10289 |
| ARHGAP21 | NM_020824 | 0.15434 | 0.16077 | 0.03215 | 0.21865 |
| MRPL55 | NM_181456 | 0.11576 | 0.10932 | 0.29904 | 0.04180 |
| RPN2 | NM_001135771 | 0.39550 | 0.16399 | 0.00000 | 0.00965 |
| UBA5 | NM_198329 | 0.23794 | 0.30868 | 0.00643 | 0.01608 |
| ARHGAP27 | NM_001282290 | 0.02894 | 0.01929 | 0.24437 | 0.27653 |
| STAP2 | NM_017720 | 0.11576 | 0.22186 | 0.08039 | 0.15113 |
| OR5H6 | NM_001005479 | 0.00000 | 0.00965 | 0.18650 | 0.37299 |
| RNF222 | NM_001146684 | 0.27010 | 0.24116 | 0.01608 | 0.04502 |
| AATF | NM_012138 | 0.08360 | 0.13183 | 0.08682 | 0.27010 |
| C2orf50 | NM_182500 | 0.20579 | 0.24116 | 0.04180 | 0.08360 |
| DLX1 | NM_178120 | 0.36334 | 0.19614 | 0.00322 | 0.00965 |
| TMCO2 | NM_001008740 | 0.09968 | 0.16077 | 0.11576 | 0.19614 |
| FNDC3A | NM_001079673 | 0.26367 | 0.25080 | 0.01608 | 0.04180 |
| NRG1 | NM_001159996 | 0.05466 | 0.13826 | 0.01929 | 0.36013 |
| SEPTIN9 | NM_001113495 | 0.14469 | 0.32154 | 0.01929 | 0.08682 |
| PTPMT1 | NM_001143984 | 0.03859 | 0.06431 | 0.28617 | 0.18328 |
| DPT | NM_001937 | 0.08682 | 0.17042 | 0.17685 | 0.13826 |
| RSPRY1 | NM_133368 | 0.02251 | 0.00965 | 0.16720 | 0.37299 |
| KANSL1L | NM_152519 | 0.04180 | 0.13826 | 0.09325 | 0.29904 |
| SNX32 | NM_152760 | 0.32476 | 0.21543 | 0.00322 | 0.02894 |
| TXLNA | NM_175852 | 0.03859 | 0.00965 | 0.21865 | 0.30547 |
| KRTAP8-1 | NM_175857 | 0.17363 | 0.23151 | 0.11254 | 0.05466 |
| CLIC6 | NM_053277 | 0.14148 | 0.18650 | 0.22186 | 0.02251 |
| EXOSC8 | NM_181503 | 0.16077 | 0.31833 | 0.04180 | 0.05466 |
| OR9G4 | NM_001005284 | 0.27974 | 0.15113 | 0.07395 | 0.07074 |
| SEPTIN5 | NM_001009939 | 0.03859 | 0.21543 | 0.03537 | 0.28617 |
| VAT1L | NM_020927 | 0.13183 | 0.21543 | 0.11897 | 0.10932 |
| TEX11 | NM_031276 | 0.05145 | 0.09968 | 0.26367 | 0.16077 |
| WDR7 | NM_052834 | 0.30868 | 0.13826 | 0.08682 | 0.04180 |
| C9orf169 | NM_199001 | 0.11576 | 0.06431 | 0.26367 | 0.13183 |
| UGT2B15 | NM_001076 | 0.18328 | 0.13826 | 0.06752 | 0.18650 |
| LRRC43 | NM_001098519 | 0.13826 | 0.09325 | 0.18006 | 0.16720 |
| CHID1 | NM_001142675 | 0.16077 | 0.12540 | 0.21543 | 0.07717 |
| ELAVL4 | NM_001144775 | 0.31833 | 0.21543 | 0.01286 | 0.03215 |
| MBNL3 | NM_001170701 | 0.23151 | 0.08360 | 0.08360 | 0.18006 |
| TET3 | NM_001287491 | 0.11897 | 0.32154 | 0.03859 | 0.09968 |
| DIRAS2 | NM_017594 | 0.15113 | 0.09003 | 0.08360 | 0.25402 |
| KCNN2 | NM_170775 | 0.21865 | 0.10932 | 0.08039 | 0.17042 |
| MOBP | NM_182935 | 0.00965 | 0.01286 | 0.21865 | 0.33762 |
| LSP1 | NM_001242932 | 0.27010 | 0.19936 | 0.03215 | 0.07717 |
| RUNDC3A | NM_006695 | 0.30868 | 0.21865 | 0.02251 | 0.02894 |
| TDRD10 | NM_182499 | 0.26688 | 0.28296 | 0.01608 | 0.01608 |
| ID1 | NM_181353 | 0.19936 | 0.35048 | 0.01929 | 0.01286 |
| REPS1 | NM_001128617 | 0.11576 | 0.16077 | 0.14791 | 0.15756 |
| TMED5 | NM_001167830 | 0.15434 | 0.19614 | 0.09325 | 0.13826 |
| ZC4H2 | NM_001178033 | 0.21222 | 0.32154 | 0.01608 | 0.03215 |
| ETV6 | NM_001987 | 0.16720 | 0.13826 | 0.08360 | 0.19293 |
| PIK3C2A | NM_002645 | 0.19936 | 0.23151 | 0.09646 | 0.05466 |
| HS3ST4 | NM_006040 | 0.14469 | 0.09003 | 0.06109 | 0.28617 |
| SAC3D1 | NM_013299 | 0.12540 | 0.10932 | 0.04180 | 0.30547 |
| CHST6 | NM_021615 | 0.25723 | 0.25723 | 0.01286 | 0.05466 |
| LCE1F | NM_178354 | 0.27010 | 0.23794 | 0.03215 | 0.04180 |
| VSX2 | NM_182894 | 0.03215 | 0.00965 | 0.27010 | 0.27010 |
| SULF2 | NM_198596 | 0.08682 | 0.09325 | 0.14791 | 0.25402 |
| TRAK1 | NM_001265609 | 0.16077 | 0.12219 | 0.07074 | 0.23151 |
| ALDH8A1 | NM_022568 | 0.09003 | 0.21865 | 0.11576 | 0.16077 |
| C20orf173 | NM_001145350 | 0.23794 | 0.33119 | 0.01286 | 0.00322 |
| CFLAR | NM_001202517 | 0.27010 | 0.29582 | 0.00322 | 0.01608 |
| METTL21A | NM_145280 | 0.08360 | 0.40836 | 0.01608 | 0.07717 |
| RIN2 | NM_001242581 | 0.07074 | 0.04180 | 0.10611 | 0.36656 |
| PSMC1 | NM_002802 | 0.07074 | 0.08360 | 0.02251 | 0.40836 |
| TSPY2 | NM_022573 | 0.08039 | 0.14791 | 0.29582 | 0.06109 |
| TTC24 | NM_001105669 | 0.27010 | 0.26688 | 0.01286 | 0.03859 |
| SLC6A8 | NM_005629 | 0.13183 | 0.42122 | 0.01929 | 0.01608 |
| RGL3 | NM_001035223 | 0.26045 | 0.20900 | 0.08682 | 0.03215 |
| CCDC104 | NM_001282761 | 0.08360 | 0.19614 | 0.23473 | 0.07395 |
| CAST | NM_001284212 | 0.03859 | 0.03215 | 0.39228 | 0.12540 |
| PLN | NM_002667 | 0.03859 | 0.08360 | 0.24437 | 0.22186 |
| NAIP | NM_004536 | 0.02251 | 0.16077 | 0.23473 | 0.17042 |
| IVL | NM_005547 | 0.23151 | 0.21543 | 0.08682 | 0.05466 |
| MITD1 | NM_138798 | 0.04180 | 0.11576 | 0.18650 | 0.24437 |
| NR1H4 | NM_001206993 | 0.04502 | 0.02894 | 0.26367 | 0.25080 |
| NUDT10 | NM_153183 | 0.15113 | 0.25080 | 0.08682 | 0.09968 |
| ADCYAPIR1 | NM_001199637 | 0.06752 | 0.09003 | 0.22830 | 0.20579 |
| MAP2K5 | NM_001206804 | 0.13183 | 0.19614 | 0.05788 | 0.20579 |
| DLAT | NM_001931 | 0.26045 | 0.24759 | 0.04180 | 0.04180 |
| MAP3K11 | NM_002419 | 0.16720 | 0.09003 | 0.06431 | 0.27010 |
| FIGF | NM_004469 | 0.23151 | 0.27010 | 0.08682 | 0.00322 |
| GGN | NM_152657 | 0.00000 | 0.00965 | 0.27010 | 0.31190 |
| DNAJC17 | NM_018163 | 0.13183 | 0.09003 | 0.17363 | 0.19614 |
| FGD6 | NM_018351 | 0.10289 | 0.12219 | 0.15756 | 0.20900 |
| DNAJC25- | NM_004125 | 0.11897 | 0.10289 | 0.17685 | 0.19293 |
| GNG10 | | | | | |
| LRRC36 | NM_001161575 | 0.32154 | 0.24116 | 0.01608 | 0.01608 |
| GALK2 | NM_001001556 | 0.08360 | 0.08360 | 0.15756 | 0.27010 |
| FAM213B | NM_001195736 | 0.16077 | 0.13826 | 0.12540 | 0.17042 |
| SERF2 | NM_001199878 | 0.00000 | 0.00322 | 0.44051 | 0.15113 |
| ACP6 | NM_016361 | 0.31511 | 0.27010 | 0.00643 | 0.00322 |
| ANKRD13C | NM_030816 | 0.20900 | 0.08360 | 0.03215 | 0.27010 |
| KIAA1958 | NM_133465 | 0.31833 | 0.16720 | 0.07395 | 0.03537 |
| TIGD6 | NM_001243253 | 0.14148 | 0.06752 | 0.09646 | 0.28939 |
| BIVM-ERCC5 | NM_001204425 | 0.00000 | 0.01286 | 0.26367 | 0.32154 |
| OSBPL8 | NM_020841 | 0.27974 | 0.08039 | 0.08682 | 0.15113 |
| TANC2 | NM_025185 | 0.16077 | 0.09003 | 0.23473 | 0.11254 |
| OR6K6 | NM_001005184 | 0.39228 | 0.20257 | 0.00000 | 0.00322 |
| CREM | NM_182724 | 0.23473 | 0.21543 | 0.00965 | 0.13826 |
| MRPL30 | NM_145212 | 0.07074 | 0.18006 | 0.17363 | 0.17363 |
| SPATA13 | NM_153023 | 0.17685 | 0.17363 | 0.07074 | 0.17685 |
| METTL20 | NM_001135863 | 0.17685 | 0.08682 | 0.28617 | 0.05145 |
| DEFB133 | NM_001166478 | 0.10289 | 0.08682 | 0.08682 | 0.32476 |
| HN1 | NM_001288609 | 0.18650 | 0.05466 | 0.27331 | 0.08682 |
| CD36 | NM_001289908 | 0.24759 | 0.32476 | 0.00322 | 0.02572 |
| IL13 | NM_002188 | 0.15756 | 0.44051 | 0.00322 | 0.00000 |
| WBSCR16 | NM_148842 | 0.39228 | 0.17042 | 0.01286 | 0.02572 |
| EBPL | NM_001278636 | 0.10289 | 0.13183 | 0.16077 | 0.20579 |
| FAM9C | NM_174901 | 0.23794 | 0.21543 | 0.06752 | 0.08039 |
| METTL13 | NM_015935 | 0.24759 | 0.21543 | 0.12540 | 0.01608 |
| OXR1 | NM_001198532 | 0.02251 | 0.01286 | 0.40836 | 0.16077 |
| MDH1 | NM_001199112 | 0.14148 | 0.09003 | 0.27010 | 0.10289 |
| CARD8 | NM_014959 | 0.25080 | 0.30868 | 0.00643 | 0.03859 |
| FCRL6 | NM_001004310 | 0.04180 | 0.05466 | 0.21865 | 0.28939 |
| QTRTD1 | NM_024638 | 0.10289 | 0.19614 | 0.12540 | 0.18006 |
| PSMB1 | NM_002793 | 0.16077 | 0.15434 | 0.01608 | 0.27653 |
| APOL2 | NM_030882 | 0.16077 | 0.11576 | 0.31833 | 0.01286 |
| OR2M5 | NM_001004690 | 0.14148 | 0.10289 | 0.09325 | 0.27010 |
| EIF2AK2 | NM_001135652 | 0.11897 | 0.15756 | 0.14791 | 0.18328 |
| AQP8 | NM_001169 | 0.16077 | 0.42122 | 0.00322 | 0.02251 |
| DEF8 | NM_001242821 | 0.35691 | 0.15756 | 0.01929 | 0.07395 |
| CLEC19A | NM_001256720 | 0.11576 | 0.13826 | 0.01608 | 0.33762 |
| TMED2 | NM_006815 | 0.08360 | 0.09646 | 0.12862 | 0.29904 |
| TAC1 | NM_013997 | 0.16077 | 0.09646 | 0.10611 | 0.24437 |
| TOLLIP | NM_019009 | 0.25080 | 0.12219 | 0.14148 | 0.09325 |
| ARHGAP18 | NM_033515 | 0.01286 | 0.02251 | 0.38585 | 0.18650 |
| TMEM255B | NM_182614 | 0.19936 | 0.35691 | 0.02251 | 0.02894 |
| KIAA1456 | NM_001099677 | 0.27010 | 0.30868 | 0.00322 | 0.02572 |
| PLK3 | NM_004073 | 0.25723 | 0.30868 | 0.00643 | 0.03537 |
| KIAA0368 | NM_001080398 | 0.14148 | 0.17363 | 0.07074 | 0.22186 |
| FAM163B | NM_001080515 | 0.14469 | 0.09003 | 0.15434 | 0.22186 |
| DTNA | NM_001198944 | 0.15434 | 0.21543 | 0.20257 | 0.03859 |
| CBFA2T3 | NM_005187 | 0.02251 | 0.00965 | 0.11897 | 0.45981 |
| STAMBP | NM_006463 | 0.00000 | 0.00000 | 0.10289 | 0.50804 |
| ASB17 | NM_080868 | 0.31511 | 0.25080 | 0.00322 | 0.04180 |
| KIAA1804 | NM_032435 | 0.10611 | 0.21543 | 0.18650 | 0.10289 |
| AK3 | NM_001199853 | 0.14469 | 0.12219 | 0.07074 | 0.27653 |
| MDM2 | NM_002392 | 0.12540 | 0.21543 | 0.02572 | 0.24759 |
| SRM | NM_003132 | 0.06109 | 0.12540 | 0.18328 | 0.24437 |
| PAIP2B | NM_020459 | 0.06109 | 0.07395 | 0.28617 | 0.19293 |
| EN1 | NM_001426 | 0.27974 | 0.32476 | 0.00000 | 0.01286 |
| ATP11C | NM_173694 | 0.36334 | 0.23473 | 0.00643 | 0.01286 |
| HTR2A | NM_001165947 | 0.03537 | 0.16077 | 0.22830 | 0.19293 |
| OSBPL3 | NM_145322 | 0.12219 | 0.11576 | 0.21865 | 0.16077 |
| PHF21B | NM_001135862 | 0.30868 | 0.23151 | 0.04180 | 0.03537 |
| PSMB4 | NM_002796 | 0.01929 | 0.04502 | 0.28296 | 0.27010 |
| EIF4EBP1 | NM_004095 | 0.08360 | 0.23151 | 0.28939 | 0.01286 |
| FRMPD4 | NM_014728 | 0.08682 | 0.12540 | 0.08682 | 0.32154 |
| PCDHGB3 | NM_018924 | 0.06752 | 0.10289 | 0.12540 | 0.32476 |
| LXN | NM_020169 | 0.07074 | 0.16720 | 0.09325 | 0.28939 |
| OR2T1 | NM_030904 | 0.00965 | 0.06109 | 0.30868 | 0.24116 |
| C17orf62 | NM_001193655 | 0.31511 | 0.29260 | 0.00322 | 0.01286 |
| FOXP1 | NM_001244813 | 0.18971 | 0.10289 | 0.17042 | 0.16077 |
| SUPT4H1 | NM_003168 | 0.14791 | 0.23151 | 0.22830 | 0.01608 |
| PDE4D | NM_006203 | 0.27010 | 0.26688 | 0.01608 | 0.07074 |
| MAL | NM_022440 | 0.00000 | 0.01286 | 0.44373 | 0.16720 |
| PDC | NM_022576 | 0.03859 | 0.08360 | 0.06752 | 0.43408 |
| MEF2C | NM_001193349 | 0.14148 | 0.10611 | 0.08682 | 0.28939 |
| IL36RN | NM_173170 | 0.13505 | 0.12862 | 0.07074 | 0.28939 |
| NSMCE2 | NM_173685 | 0.32476 | 0.24437 | 0.01608 | 0.04180 |
| MBOAT7 | NM_001146056 | 0.16077 | 0.09003 | 0.12540 | 0.25080 |
| ALDH18A1 | NM_002860 | 0.08360 | 0.09003 | 0.26367 | 0.18971 |
| MARCHF1 | NM_017923 | 0.31511 | 0.21543 | 0.01608 | 0.08039 |
| MS4A2 | NM_001256916 | 0.16077 | 0.30868 | 0.02572 | 0.13183 |
| SCML4 | NM_001286409 | 0.03859 | 0.09003 | 0.36013 | 0.13826 |
| KLRC2 | NM_002260 | 0.00643 | 0.04180 | 0.30868 | 0.27010 |
| FBXL12 | NM_017703 | 0.30868 | 0.31833 | 0.00000 | 0,00000 |
| AMPD2 | NM_001257360 | 0.09968 | 0.15756 | 0.08039 | 0.28939 |
| USP5 | NM_003481 | 0.08360 | 0.13826 | 0.14791 | 0.25723 |
| TSPAN5 | NM_005723 | 0.11576 | 0.35048 | 0.02251 | 0.13826 |
| CNBD1 | NM_173538 | 0.26367 | 0.22830 | 0.11576 | 0.01929 |
| MMP11 | NM_005940 | 0.13183 | 0.13505 | 0.30868 | 0.05466 |
| EBF2 | NM_022659 | 0.00965 | 0.08360 | 0.12862 | 0.40836 |
| MXI1 | NM_130439 | 0.32154 | 0.30868 | 0.00000 | 0.00000 |
| CERS2 | NM_181746 | 0.15434 | 0.35048 | 0.09003 | 0.03537 |
| SPATS2L | NM_001282735 | 0.00643 | 0.07395 | 0.13505 | 0.41479 |
| CASKIN1 | NM_020764 | 0.41479 | 0.16077 | 0.02251 | 0.03215 |
| ZNF200 | NM_003454 | 0.09646 | 0.06752 | 0.19614 | 0.27010 |
| CYP3A43 | NM_057096 | 0.14469 | 0.22830 | 0.06431 | 0.19293 |
| ZEB1 | NM_001174094 | 0.24759 | 0.35691 | 0.01286 | 0.01608 |
| MYADM | NM_001020818 | 0.16077 | 0.21865 | 0.09325 | 0.16077 |
| RAB41 | NM_001032726 | 0.12540 | 0.13183 | 0.18006 | 0.19614 |
| GPR149 | NM_001038705 | 0.32154 | 0.27010 | 0.00643 | 0.03537 |
| KLHL3 | NM_001257194 | 0.03859 | 0.09325 | 0.11576 | 0.38585 |
| MYRF | NM_013279 | 0.16077 | 0.07395 | 0.31833 | 0.08039 |
| TMEM141 | NM_032928 | 0.18328 | 0.28296 | 0.00000 | 0.16720 |
| ACPT | NM_033068 | 0.19614 | 0.38264 | 0.01608 | 0.03859 |
| HAUS8 | NM_033417 | 0.31511 | 0.13826 | 0.12540 | 0.05466 |
| FBLIM1 | NM_001024215 | 0.22830 | 0.24116 | 0.15434 | 0.00965 |
| SHFM1 | NM_006304 | 0.09003 | 0.11576 | 0.17685 | 0.25080 |
| FGD3 | NM_033086 | 0.14148 | 0.18650 | 0.17685 | 0.12862 |
| PEX11B | NM_003846 | 0.16399 | 0.21543 | 0.08682 | 0.17042 |
| WDR1 | NM_017491 | 0.10289 | 0.30868 | 0.01608 | 0.20900 |
| SLC25A36 | NM_018155 | 0.09003 | 0.26688 | 0.09325 | 0.18650 |
| DDRGK1 | NM_023935 | 0.03859 | 0.08682 | 0.23473 | 0.27653 |
| CDC26 | NM_139286 | 0.31833 | 0.19614 | 0.08682 | 0.03537 |
| ATP6V1H | NM_213620 | 0.12540 | 0.10289 | 0.17685 | 0.23151 |
| GOLGA8J | NM_001282472 | 0.19936 | 0.33119 | 0.00643 | 0.09968 |
| PDCL3 | NM_024065 | 0.19936 | 0.21543 | 0.05788 | 0.16399 |
| CD36 | NM_001289909 | 0.23151 | 0.30868 | 0.06109 | 0.03537 |
| ALG10 | NM_032834 | 0.08360 | 0.18650 | 0.22830 | 0.13826 |
| KCNC3 | NM_004977 | 0.16077 | 0.16077 | 0.12540 | 0.19293 |
| TRIL | NM_014817 | 0.14148 | 0.31190 | 0.01608 | 0.17042 |
| PIH1D1 | NM_017916 | 0.23151 | 0.18971 | 0.08682 | 0.13183 |
| MGAT5B | NM_001199172 | 0.32476 | 0.30868 | 0.00643 | 0.00000 |
| MARCHF5 | NM_017824 | 0.23794 | 0.20579 | 0.18006 | 0.01608 |
| TM7SF2 | NM_001277233 | 0.30868 | 0.21865 | 0.07074 | 0.04180 |
| OARD1 | NM_145063 | 0.25723 | 0.19936 | 0.06752 | 0.11576 |
| IL18BP | NM_001145057 | 0.14148 | 0.05466 | 0.09646 | 0.34727 |
| EEF1A2 | NM_001958 | 0.14148 | 0.06431 | 0.12862 | 0.30547 |
| SIDT1 | NM_017699 | 0.27010 | 0.08360 | 0.10611 | 0.18006 |
| KIRREL | NM_018240 | 0.23151 | 0.15113 | 0.22830 | 0.02894 |
| MAK16 | NM_032509 | 0.16077 | 0.09003 | 0.02251 | 0.36656 |
| SSX5 | NM_175723 | 0.15113 | 0.22830 | 0.15756 | 0.10289 |
| TOX | NM_014729 | 0.16077 | 0.03215 | 0.16399 | 0.28617 |
| CIRH1A | NM_032830 | 0.26367 | 0.19614 | 0.12862 | 0.05466 |
| RICTOR | NM_152756 | 0.27010 | 0.35691 | 0.01286 | 0.00322 |
| PNLIPRP3 | NM_001011709 | 0.27653 | 0.10932 | 0.07074 | 0.18650 |
| CXorf27 | NM_012274 | 0.14469 | 0.14791 | 0.04180 | 0.30868 |
| CABYR | NM_153770 | 0.10611 | 0.03859 | 0.41158 | 0.08682 |
| GLB1 | NM_001135602 | 0.23794 | 0.18328 | 0.04180 | 0.18006 |
| MCAM | NM_006500 | 0.11576 | 0.08360 | 0.41801 | 0.02572 |
| ZBTB25 | NM_006977 | 0.26045 | 0.23794 | 0.02251 | 0.12219 |
| ZDHHC1 | NM_013304 | 0.13826 | 0.24116 | 0.04180 | 0.22186 |
| ULBP2 | NM_025217 | 0.17363 | 0.13826 | 0.16077 | 0.17042 |
| FOXN1 | NM_003593 | 0.23151 | 0.36013 | 0.04180 | 0.01286 |
| PLA2G2C | NM_001105572 | 0.16077 | 0.34727 | 0.08682 | 0.05145 |
| BMPR2 | NM_001204 | 0.16720 | 0.23151 | 0.00643 | 0.24116 |
| C18orf54 | NM_001288982 | 0.31833 | 0.24116 | 0.07074 | 0.01608 |
| GBP6 | NM_198460 | 0.11897 | 0.09003 | 0.27010 | 0.16720 |
| TRIM60 | NM_001258025 | 0.09003 | 0.42122 | 0.06752 | 0.06752 |
| ACADM | NM_001286043 | 0.31511 | 0.27010 | 0.01929 | 0.04180 |
| EIF3H | NM_003756 | 0.18328 | 0.27010 | 0.07395 | 0.11897 |
| TPP1 | NM_000391 | 0.07717 | 0.07074 | 0.07074 | 0.42765 |
| SPARCL1 | NM_001128310 | 0.00000 | 0.00965 | 0.45016 | 0.18650 |
| SLC39A1 | NM_001271958 | 0.19293 | 0.18650 | 0.08682 | 0.18006 |
| HLA-DMA | NM_006120 | 0.00000 | 0.01929 | 0.32154 | 0.30547 |
| HMHA1 | NM_012292 | 0.02251 | 0.00965 | 0.20579 | 0.40836 |
| BPIFB2 | NM_025227 | 0.02251 | 0.00643 | 0.45016 | 0.16720 |
| PKHD1 | NM_138694 | 0.05466 | 0.02572 | 0.10611 | 0.45981 |
| DPYSL3 | NM_001387 | 0.17685 | 0.13826 | 0.27010 | 0.06109 |
| ACADL | NM_001608 | 0.10289 | 0.17363 | 0.07074 | 0.29904 |
| AIFM3 | NM_001146288 | 0.24759 | 0.36334 | 0.02251 | 0.01608 |
| ZNF548 | NM_001172773 | 0.00643 | 0.00322 | 0.39228 | 0.24759 |
| TRPV1 | NM_080704 | 0.23151 | 0.16077 | 0.08682 | 0.17042 |
| MROH7 | NM_001039464 | 0.30868 | 0.26688 | 0.03215 | 0.04180 |
| SMIM5 | NM_001162995 | 0.15434 | 0.04502 | 0.11576 | 0.33441 |
| CCDC93 | NM_019044 | 0.09968 | 0.07395 | 0.27010 | 0.20579 |
| PRELID2 | NM_138492 | 0.06431 | 0.13183 | 0.15756 | 0.29582 |
| IGJ | NM_144646 | 0.11576 | 0.30868 | 0.03215 | 0.19293 |
| ELL2 | NM_012081 | 0.14469 | 0.30225 | 0.17685 | 0.02572 |
| NCBP1 | NM_002486 | 0.32476 | 0.19293 | 0.08682 | 0.04823 |
| PID1 | NM_017933 | 0.17042 | 0.09325 | 0.12862 | 0.26045 |
| RBMS3 | NM_001177711 | 0.25080 | 0.09003 | 0.08360 | 0.22830 |
| STX10 | NM_001271609 | 0.32154 | 0.15434 | 0.14469 | 0.03215 |
| EPB41L3 | NM_001281535 | 0.17363 | 0.16077 | 0.27010 | 0.04823 |
| SECTM1 | NM_003004 | 0.16399 | 0.12862 | 0.25723 | 0.10289 |
| PTPRO | NM_030667 | 0.15113 | 0.14791 | 0.26367 | 0.09003 |
| ZNF566 | NM_032838 | 0.27010 | 0.24116 | 0.09968 | 0.04180 |
| ABCA2 | NM_212533 | 0.15434 | 0.18328 | 0.09325 | 0.22186 |
| OPRM1 | NM_001145287 | 0.23151 | 0.34727 | 0.02251 | 0.05145 |
| CEP83 | NM_001042399 | 0.11576 | 0.11576 | 0.09003 | 0.33441 |
| FAM156B | NM_001099684 | 0.16720 | 0.30868 | 0.04180 | 0.13826 |
| RNF19B | NM_001127361 | 0.09003 | 0.01929 | 0.22186 | 0.32476 |
| APC | NM_001127510 | 0.16720 | 0.21543 | 0.00322 | 0.27010 |
| TRAPPC3 | NM_001270895 | 0.25402 | 0.09003 | 0.11897 | 0.19293 |
| NGDN | NM_015514 | 0.23151 | 0.17363 | 0.11897 | 0.13183 |
| IFT140 | NM_014714 | 0.10611 | 0.29582 | 0.18006 | 0.07395 |
| ZNF518A | NM_014803 | 0.01286 | 0.00965 | 0.08682 | 0.54984 |
| DGCR6 | NM_005675 | 0.13183 | 0.05466 | 0.26688 | 0.20579 |
| STS | NM_000351 | 0.26367 | 0.17363 | 0.01608 | 0.20579 |
| DPP9 | NM_139159 | 0.27010 | 0.30547 | 0.01608 | 0.06752 |
| KLRC1 | NM_002259 | 0.00965 | 0.01608 | 0.26688 | 0.36656 |
| IPO5 | NM_002271 | 0.20900 | 0.17363 | 0.12862 | 0.14791 |
| WNT5B | NM_032642 | 0.27653 | 0.38264 | 0.00000 | 0.00000 |
| ARHGEF1 | NM_199002 | 0.16077 | 0.22830 | 0.21543 | 0.05466 |
| KCNAB3 | NM_004732 | 0.13826 | 0.22508 | 0.14791 | 0.14791 |
| KIAA1024 | NM_015206 | 0.15756 | 0.13826 | 0.14148 | 0.22186 |
| HEATR2 | NM_017802 | 0.19614 | 0.02251 | 0.06752 | 0.37299 |
| SLAIN1 | NM_001242868 | 0.20900 | 0.17042 | 0.19614 | 0.08682 |
| CD84 | NM_001184879 | 0.09003 | 0.08360 | 0.36013 | 0.12862 |
| PACS2 | NM_001243127 | 0.31511 | 0.19936 | 0.09325 | 0.05466 |
| BDKRB1 | NM_000710 | 0.14148 | 0.09003 | 0.20257 | 0.22830 |
| C6orf58 | NM_001010905 | 0.03859 | 0.08360 | 0.27010 | 0.27010 |
| SULF1 | NM_001128205 | 0.11897 | 0.07395 | 0.22830 | 0.24116 |
| CIZ1 | NM_001131015 | 0.31511 | 0.09003 | 0.17685 | 0.08039 |
| TEX33 | NM_001163857 | 0.19614 | 0.04823 | 0.22508 | 0.19293 |
| GHR | NM_001242406 | 0.18650 | 0.42122 | 0.00643 | 0.04823 |
| NME3 | NM_002513 | 0.32154 | 0.26688 | 0.02572 | 0.04823 |
| KLC1 | NM_005552 | 0.01286 | 0.00965 | 0.18006 | 0.45981 |
| RASGRF2 | NM_006909 | 0.11576 | 0.09003 | 0.07074 | 0.38585 |
| MPHOSPH8 | NM_017520 | 0.16720 | 0.18328 | 0.14148 | 0.17042 |
| PCDHGA4 | NM_018917 | 0.08360 | 0.02572 | 0.36013 | 0.19293 |
| CARF | NM_024744 | 0.18971 | 0.25080 | 0.01608 | 0.20579 |
| BMP2K | NM_198892 | 0.03859 | 0.00965 | 0.34084 | 0.27331 |
| SCAP | NM_012235 | 0.27010 | 0.35691 | 0.00643 | 0.02894 |
| PHTF1 | NM_006608 | 0.14469 | 0.11576 | 0.06752 | 0.33441 |
| PTRH1 | NM_001002913 | 0.19614 | 0.20900 | 0.09325 | 0.16720 |
| GRIN2A | NM_001134408 | 0.12540 | 0.08360 | 0.18006 | 0.27653 |
| KCNH1 | NM_002238 | 0.01286 | 0.09003 | 0.19614 | 0.36656 |
| NUP188 | NM_015354 | 0.01608 | 0.00643 | 0.37621 | 0.26688 |
| WRNIP1 | NM_130395 | 0.04180 | 0.08039 | 0.15113 | 0.39228 |
| LTA4H | NM_001256643 | 0.23794 | 0.16720 | 0.18328 | 0.07717 |
| UBIAD1 | NM_013319 | 0.35691 | 0.21222 | 0.09325 | 0.00322 |
| CDKN1A | NM_078467 | 0.02894 | 0.04502 | 0.20257 | 0.38907 |
| RNF165 | NM_152470 | 0.33762 | 0.27010 | 0.00643 | 0.05145 |
| TMEM233 | NM_001136534 | 0.06752 | 0.10289 | 0.32797 | 0.16720 |
| CCSER1 | NM_207491 | 0.10289 | 0.10289 | 0.37942 | 0.08039 |
| LOC200726 | NM_001102659 | 0.23151 | 0.27010 | 0.12540 | 0.04180 |
| DDX19B | NM_001257173 | 0.32154 | 0.08039 | 0.16720 | 0.09968 |
| SBNO2 | NM_014963 | 0.01286 | 0.01608 | 0.21865 | 0.42122 |
| SSX7 | NM_173358 | 0.27010 | 0.36013 | 0.01608 | 0.02251 |
| LRRC27 | NM_001143757 | 0.16720 | 0.01608 | 0.18006 | 0.30547 |
| PHACTR3 | NM_001199505 | 0.00965 | 0.08360 | 0.28617 | 0.28939 |
| NDUFAB1 | NM_005003 | 0.39550 | 0.26688 | 0.00643 | 0.00000 |
| APBB1IP | NM_019043 | 0.04180 | 0.07395 | 0.30868 | 0.24437 |
| TENC1 | NM_198316 | 0.23794 | 0.09646 | 0.08682 | 0.24759 |
| OGN | NM_014057 | 0.27974 | 0.34727 | 0.00643 | 0.03537 |
| UTP3 | NM_020368 | 0.06109 | 0.08360 | 0.23473 | 0.28939 |
| TSHZ1 | NM_005786 | 0.10289 | 0.09003 | 0.17685 | 0.29904 |
| PPIP5K2 | NM_015216 | 0.16077 | 0.06431 | 0.22186 | 0.22186 |
| PSMB8 | NM_148919 | 0.25080 | 0.34727 | 0.01929 | 0.05145 |

TABLE 3B
FDR of the CRISPRa chronic screening results. Gene targets are in ranked order.
Gene names, RefSeq IDs, and estimated FDR using negative control genes (see
Methods) for each of the top 1000 genes in the screening bioreps are listed.

| | | Chronic | Chronic | Chronic | Chronic | Chronic | Chronic |
|---|---|---|---|---|---|---|---|
| | | Round 1 | Round 1 | Round 2 | Round 2 | Round 3 | Round 3 |
| Gene | id | biorep 1 | biorep 2 | biorep 1 | biorep 2 | biorep 1 | biorep 2 |
| B3GNT2 | NM_006577 | 0.00322 | 0.00000 | 0.00000 | 0.00000 | 0.00000 | 0.00000 |
| JUNB | NM_002229 | 0.00000 | 0.00322 | 0.00000 | 0.00965 | 0.00000 | 0.01286 |
| INO80 | NM_017553 | 0.00000 | 0.00965 | 0.00000 | 0.00965 | 0.03537 | 0.00965 |
| F8A2 | NM_001007523 | 0.00965 | 0.00965 | 0.00000 | 0.01608 | 0.00643 | 0.02572 |
| STK40 | NM_032017 | 0.00965 | 0.01286 | 0.00000 | 0.01286 | 0.01929 | 0.01286 |
| C2orf88 | NM_001042520 | 0.00000 | 0.01608 | 0.00643 | 0.00965 | 0.05466 | 0.00965 |
| KRBA1 | NM_001290187 | 0.00000 | 0.05145 | 0.00000 | 0.02894 | 0.00000 | 0.02894 |
| DKK2 | NM_014421 | 0.00000 | 0.02894 | 0.00000 | 0.04823 | 0.00000 | 0.03859 |
| ITLN1 | NM_017625 | 0.01929 | 0.05145 | 0.00000 | 0.02572 | 0.00000 | 0.03537 |
| IQSEC3 | NM_015232 | 0.02251 | 0.02894 | 0.02251 | 0.02572 | 0.01608 | 0.02251 |
| NIPAL1 | NM_207330 | 0.00965 | 0.01286 | 0.03859 | 0.00965 | 0.06109 | 0.01286 |
| FEZ2 | NM_005102 | 0.00000 | 0.09646 | 0.00000 | 0.02572 | 0.01286 | 0.01286 |
| WDR76 | NM_001167941 | 0.00000 | 0.09003 | 0.00000 | 0.02572 | 0.03537 | 0.01286 |
| ZNF497 | NM_001207009 | 0.00322 | 0.02894 | 0.03537 | 0.03537 | 0.06109 | 0.01286 |
| CIDEC | NM_001199623 | 0.09325 | 0.00322 | 0.04823 | 0.00965 | 0.01608 | 0.00965 |
| KCNF1 | NM_002236 | 0.02894 | 0.05145 | 0.02251 | 0.01286 | 0.04180 | 0.02251 |
| RINT1 | NM_021930 | 0.06752 | 0.00322 | 0.03859 | 0.00965 | 0.04823 | 0.01608 |
| LGALS14 | NM_203471 | 0.04823 | 0.00322 | 0.07717 | 0.00965 | 0.05466 | 0.01286 |
| ATP2A2 | NM_170665 | 0.00000 | 0.11897 | 0.00965 | 0.03537 | 0.01286 | 0.03537 |
| TMEM55B | NM_144568 | 0.04823 | 0.01286 | 0.06431 | 0.01608 | 0.05788 | 0.01608 |
| CHRND | NM_001256657 | 0.10289 | 0.01929 | 0.03859 | 0.00965 | 0.03537 | 0.01286 |
| LHX2 | NM_004789 | 0.10289 | 0.00322 | 0.04180 | 0.00965 | 0.04823 | 0.01608 |
| ZNF576 | NM_024327 | 0.02894 | 0.13505 | 0.00322 | 0.02894 | 0.01286 | 0.01608 |
| CCBL2 | NM_001008661 | 0.00965 | 0.09003 | 0.03859 | 0.02572 | 0.05466 | 0.00965 |
| GABBR1 | NM_021904 | 0.00000 | 0.17042 | 0.00000 | 0.01608 | 0.01929 | 0.02251 |
| GOLGA8M | NM_001282468 | 0.01286 | 0.02894 | 0.09646 | 0.00965 | 0.07717 | 0.00643 |
| CCDC160 | NM_001101357 | 0.00965 | 0.09646 | 0.00000 | 0.05466 | 0.00000 | 0.07395 |
| RBPMS2 | NM_194272 | 0.03859 | 0.13826 | 0.01286 | 0.00965 | 0.02894 | 0.01286 |
| CMTM3 | NM_144601 | 0.05788 | 0.00643 | 0.07395 | 0.02894 | 0.05788 | 0.02251 |
| KCNH7 | NM_033272 | 0.00000 | 0.06431 | 0.02251 | 0.03859 | 0.00965 | 0.11897 |
| YY1AP1 | NM_001198902 | 0.16399 | 0.00000 | 0.05788 | 0.00965 | 0.01608 | 0.00965 |
| ASXL3 | NM_030632 | 0.02894 | 0.02572 | 0.02572 | 0.06431 | 0.02251 | 0.09003 |
| RCAN2 | NM_001251974 | 0.01929 | 0.07717 | 0.00000 | 0.06431 | 0.00000 | 0.09968 |
| ADAMTS12 | NM_030955 | 0.08360 | 0.00322 | 0.04823 | 0.06431 | 0.00965 | 0.05466 |
| SGPP2 | NM_152386 | 0.02251 | 0.07717 | 0.01286 | 0.05145 | 0.05466 | 0.04502 |
| CCDC66 | NM_001141947 | 0.07717 | 0.02572 | 0.08039 | 0.00965 | 0.06109 | 0.01286 |
| JUN | NM_002228 | 0.01929 | 0.00322 | 0.09646 | 0.03537 | 0.09968 | 0.01608 |
| ACTG1 | NM_001199954 | 0.07074 | 0.07717 | 0.03537 | 0.05466 | 0.01608 | 0.02251 |
| MBD3L3 | NM_001164425 | 0.02894 | 0.04502 | 0.03859 | 0.07395 | 0.03859 | 0.05145 |
| MAGEA4 | NM_001011549 | 0.00000 | 0.13183 | 0.00000 | 0.04823 | 0.00000 | 0.09968 |
| RPS11 | NM_001015 | 0.06431 | 0.02572 | 0.05788 | 0.02894 | 0.08360 | 0.01929 |
| POP1 | NM_015029 | 0.09003 | 0.02894 | 0.06109 | 0.02894 | 0.06431 | 0.01286 |
| SMIM12 | NM_001164825 | 0.05145 | 0.14791 | 0.01286 | 0.01608 | 0.03859 | 0.02251 |
| RPUSD4 | NM_032795 | 0.00000 | 0.05466 | 0.00000 | 0.07717 | 0.01608 | 0.14148 |
| SCN11A | NM_014139 | 0.06752 | 0.00322 | 0.07717 | 0.03537 | 0.05466 | 0.05466 |
| FLI1 | NM_001167681 | 0.09325 | 0.00643 | 0.01286 | 0.06431 | 0.01608 | 0.09968 |
| FAM118A | NM_017911 | 0.00000 | 0.27010 | 0.00000 | 0.00965 | 0.00322 | 0.00965 |
| PCYT2 | NM_001184917 | 0.01929 | 0.09646 | 0.00000 | 0.07395 | 0.00322 | 0.09968 |
| CALD1 | NM_033140 | 0.07074 | 0.07717 | 0.03859 | 0.04502 | 0.03859 | 0.02572 |
| SSX4B | NM_001034832 | 0.04180 | 0.11576 | 0.03859 | 0.03537 | 0.04502 | 0.02251 |
| NELFA | NM_005663 | 0.03537 | 0.03859 | 0.03859 | 0.06752 | 0.05466 | 0.06431 |
| CDKN2A | NM_000077 | 0.00965 | 0.05145 | 0.08039 | 0.01608 | 0.11576 | 0.02894 |
| STYX | NM_001130701 | 0.03215 | 0.01929 | 0.06752 | 0.03215 | 0.06109 | 0.09003 |
| ZBTB22 | NM_001145338 | 0.10289 | 0.00965 | 0.10611 | 0.00965 | 0.06431 | 0.01286 |
| ATG16L2 | NM_033388 | 0.05466 | 0.00643 | 0.03859 | 0.04502 | 0.05466 | 0.10932 |
| CD109 | NM_001159587 | 0.11254 | 0.00643 | 0.12219 | 0.00965 | 0.05466 | 0.00643 |
| ACTA1 | NM_001100 | 0.04823 | 0.08039 | 0.09325 | 0.02894 | 0.05466 | 0.01286 |
| BEX2 | NM_001168401 | 0.00965 | 0.07717 | 0.00322 | 0.04502 | 0.03537 | 0.14791 |
| SHROOM3 | NM_020859 | 0.00000 | 0.20257 | 0.00000 | 0.04823 | 0.00000 | 0.06752 |
| ERMN | NM_001009959 | 0.01929 | 0.19293 | 0.00000 | 0.03537 | 0.00000 | 0.07395 |
| SRC | NM_198291 | 0.05788 | 0.20579 | 0.00643 | 0.02894 | 0.00965 | 0.01608 |
| TACR3 | NM_001059 | 0.09003 | 0.00322 | 0.09968 | 0.00965 | 0.11576 | 0.00643 |
| NYNRIN | NM_025081 | 0.14469 | 0.09325 | 0.00000 | 0.04502 | 0.00643 | 0.03537 |
| FCER1A | NM_002001 | 0.02894 | 0.10611 | 0.03859 | 0.05466 | 0.03859 | 0.05788 |
| EFCAB1 | NM_001142857 | 0.03537 | 0.04502 | 0.08039 | 0.02894 | 0.12862 | 0.01608 |
| CYB5R3 | NM_001171661 | 0.04502 | 0.09646 | 0.06752 | 0.04502 | 0.06752 | 0.01608 |
| PPP2R2C | NM_001206995 | 0.03537 | 0.00322 | 0.09325 | 0.02572 | 0.16077 | 0.02251 |
| MCRS1 | NM_006337 | 0.13505 | 0.07717 | 0.03859 | 0.03215 | 0.05466 | 0.01608 |
| LRFN1 | NM_020862 | 0.07717 | 0.09646 | 0.07074 | 0.03215 | 0.05466 | 0.02894 |
| EFNA1 | NM_004428 | 0.12540 | 0.13183 | 0.03859 | 0.02572 | 0.01608 | 0.02572 |
| MACROD2 | NM_001033087 | 0.08360 | 0.16077 | 0.01929 | 0.03215 | 0.01286 | 0.05466 |
| NSL1 | NM_015471 | 0.00322 | 0.11576 | 0.00000 | 0.11576 | 0.00965 | 0.11897 |
| HGC6.3 | NM_001129895 | 0.05466 | 0.02894 | 0.03859 | 0.05788 | 0.01929 | 0.16399 |
| C11orf83 | NM_001085372 | 0.11576 | 0.02894 | 0.09968 | 0.03215 | 0.06431 | 0.02251 |
| CPEB1 | NM_030594 | 0.00322 | 0.06431 | 0.02251 | 0.07717 | 0.05466 | 0.15113 |
| RPL13 | NM_001243130 | 0.11576 | 0.07395 | 0.01608 | 0.03859 | 0.03859 | 0.09325 |
| RBBP8 | NM_002894 | 0.14791 | 0.04180 | 0.10611 | 0.00965 | 0.06109 | 0.01286 |
| CMTM4 | NM_181521 | 0.00965 | 0.01929 | 0.04180 | 0.07395 | 0.09968 | 0.13505 |
| ABCA4 | NM_000350 | 0.06752 | 0.08039 | 0.08039 | 0.03215 | 0.06431 | 0.05788 |
| RPL15 | NM_001253384 | 0.10289 | 0.04180 | 0.08682 | 0.00643 | 0.14791 | 0.00322 |
| SF3A2 | NM_007165 | 0.01929 | 0.22508 | 0.07074 | 0.00965 | 0.05466 | 0.00965 |
| REM1 | NM_014012 | 0.17685 | 0.01929 | 0.03859 | 0.05145 | 0.01286 | 0.09003 |
| LTN1 | NM_015565 | 0.05788 | 0.13183 | 0.07074 | 0.06431 | 0.03537 | 0.02894 |
| DISP2 | NM_033510 | 0.00000 | 0.22508 | 0.00000 | 0.07395 | 0.00000 | 0.09325 |
| NSG1 | NM_001287763 | 0.00000 | 0.08039 | 0.00643 | 0.16077 | 0.05788 | 0.09003 |
| TMEM147 | NM_032635 | 0.00000 | 0.20257 | 0.00965 | 0.04823 | 0.06109 | 0.07717 |
| FAM47A | NM_203408 | 0.00000 | 0.27010 | 0.00322 | 0.03215 | 0.04502 | 0.04823 |
| EXOC3L1 | NM_178516 | 0.04823 | 0.06109 | 0.10611 | 0.05145 | 0.06109 | 0.07074 |
| LITAF | NM_004862 | 0.04823 | 0.11576 | 0.08039 | 0.01608 | 0.13183 | 0.01286 |
| ZIC5 | NM_033132 | 0.00000 | 0.11576 | 0.03859 | 0.07074 | 0.08039 | 0.09968 |
| CCDC169 | NM_001144984 | 0.09003 | 0.05145 | 0.08039 | 0.03537 | 0.09968 | 0.05145 |
| SATB1 | NM_001195470 | 0.00000 | 0.12219 | 0.00643 | 0.09646 | 0.03537 | 0.14791 |
| EXOC4 | NM_021807 | 0.10932 | 0.09003 | 0.03859 | 0.05788 | 0.04823 | 0.06431 |
| HMGB2 | NM_001130689 | 0.00000 | 0.20257 | 0.09646 | 0.00965 | 0.10289 | 0.00322 |
| ESRRG | NM_001134285 | 0.07074 | 0.07717 | 0.09325 | 0.00965 | 0.16077 | 0.00643 |
| LTBR | NM_002342 | 0.03859 | 0.00322 | 0.18971 | 0.00965 | 0.16720 | 0.00965 |
| CLDN4 | NM_001305 | 0.00965 | 0.07717 | 0.00322 | 0.11254 | 0.02894 | 0.18650 |
| ECHDC1 | NM_018479 | 0.19936 | 0.00643 | 0.09646 | 0.02894 | 0.06109 | 0.02894 |
| WBP1L | NM_017787 | 0.04823 | 0.05466 | 0.10611 | 0.03215 | 0.15113 | 0.03537 |
| KCNQ4 | NM_004700 | 0.10289 | 0.02894 | 0.13183 | 0.02572 | 0.06431 | 0.07395 |
| NSUN3 | NM_022072 | 0.02251 | 0.04502 | 0.03859 | 0.07395 | 0.07074 | 0.17685 |
| TMEM132A | NM_017870 | 0.09003 | 0.07717 | 0.08360 | 0.01608 | 0.15113 | 0.01286 |
| DVL3 | NM_004423 | 0.05466 | 0.09003 | 0.14148 | 0.06431 | 0.06431 | 0.01608 |
| SLC25A35 | NM_201520 | 0.00965 | 0.03215 | 0.09646 | 0.07395 | 0.16720 | 0.05145 |
| MX1 | NM_001178046 | 0.00965 | 0.14469 | 0.03859 | 0.07717 | 0.05466 | 0.10932 |
| KLK10 | NM_002776 | 0.09325 | 0.13183 | 0.03859 | 0.09325 | 0.01929 | 0.05788 |
| CD97 | NM_078481 | 0.02894 | 0.00965 | 0.11897 | 0.03537 | 0.15113 | 0.09003 |
| ATG16L1 | NM_017974 | 0.08360 | 0.07717 | 0.08039 | 0.06431 | 0.10289 | 0.02572 |
| TMPRSS5 | NM_030770 | 0.03537 | 0.05466 | 0.00965 | 0.09646 | 0.05466 | 0.18650 |
| SPATA31A5 | NM_001113541 | 0.05466 | 0.03859 | 0.10932 | 0.05788 | 0.13183 | 0.04823 |
| CD151 | NM_139030 | 0.15434 | 0.15756 | 0.03859 | 0.00965 | 0.05466 | 0.02572 |
| PLEC | NM_201382 | 0.07074 | 0.10611 | 0.12219 | 0.04823 | 0.07074 | 0.02251 |
| CELF2 | NM_006561 | 0.00322 | 0.18328 | 0.00965 | 0.13826 | 0.05466 | 0.05466 |
| DUSP10 | NM_144728 | 0.12540 | 0.15434 | 0.09646 | 0.02251 | 0.01286 | 0.03537 |
| MEPE | NM_001291183 | 0.15434 | 0.02251 | 0.07395 | 0.05788 | 0.04823 | 0.09003 |
| LOC113230 | NM_001291291 | 0.07717 | 0.01286 | 0.14791 | 0.02572 | 0.16077 | 0.02251 |
| MYL6B | NM_002475 | 0.09003 | 0.03215 | 0.07074 | 0.07717 | 0.03537 | 0.14148 |
| KCNV1 | NM_014379 | 0.00965 | 0.03215 | 0.09325 | 0.06752 | 0.14148 | 0.10289 |
| CNN3 | NM_001839 | 0.03859 | 0.07717 | 0.09646 | 0.06431 | 0.09968 | 0.07074 |
| KIF3A | NM_007054 | 0.12219 | 0.02894 | 0.10932 | 0.02894 | 0.09968 | 0.05788 |
| NOL12 | NM_024313 | 0.08360 | 0.00643 | 0.07717 | 0.03215 | 0.16077 | 0.09003 |
| SLC25A1 | NM_005984 | 0.04823 | 0.18328 | 0.04502 | 0.03537 | 0.04180 | 0.09646 |
| UBE2T | NM_014176 | 0.08682 | 0.04502 | 0.10611 | 0.02572 | 0.13505 | 0.05145 |
| FAM155A | NM_001080396 | 0.00965 | 0.07717 | 0.08039 | 0.11254 | 0.05466 | 0.11897 |
| DAPL1 | NM_001017920 | 0.03859 | 0.21543 | 0.04502 | 0.04502 | 0.06109 | 0.05145 |
| NFE2 | NM_001136023 | 0.00000 | 0.17042 | 0.00000 | 0.17363 | 0.01286 | 0.09968 |
| PKIG | NM_001281444 | 0.00000 | 0.06431 | 0.01286 | 0.17685 | 0.05466 | 0.14791 |
| CCDC109B | NM_017918 | 0.16399 | 0.11897 | 0.05788 | 0.04502 | 0.04823 | 0.02251 |
| TGFBR1 | NM_001130916 | 0.12219 | 0.05145 | 0.08039 | 0.04180 | 0.06431 | 0.10289 |
| C1orf158 | NM_152290 | 0.12219 | 0.11576 | 0.12862 | 0.01608 | 0.05788 | 0.02251 |
| ZFAT | NM_001289394 | 0.15756 | 0.01286 | 0.09325 | 0.07074 | 0.06431 | 0.06752 |
| TRIM7 | NM_203293 | 0.15434 | 0.00322 | 0.13505 | 0.00965 | 0.15756 | 0.00965 |
| MTRR | NM_024010 | 0.02251 | 0.11897 | 0.00322 | 0.17685 | 0.01608 | 0.13505 |
| TAP1 | NM_000593 | 0.09003 | 0.05466 | 0.05466 | 0.05788 | 0.08682 | 0.13826 |
| SHE | NM_001010846 | 0.06752 | 0.00322 | 0.16399 | 0.00965 | 0.22508 | 0.01286 |
| TRIM50 | NM_001281451 | 0.00322 | 0.09003 | 0.09646 | 0.06752 | 0.14148 | 0.08360 |
| MPP2 | NM_001278375 | 0.15434 | 0.08682 | 0.12219 | 0.00965 | 0.10289 | 0.00965 |
| MIER1 | NM_001146112 | 0.18971 | 0.05788 | 0.06752 | 0.05788 | 0.05788 | 0.05466 |
| EDC3 | NM_025083 | 0.00000 | 0.24759 | 0.00322 | 0.07395 | 0.03537 | 0.12862 |
| GSC | NM_173849 | 0.12540 | 0.06109 | 0.04180 | 0.09003 | 0.06109 | 0.10932 |
| NPY5R | NM_006174 | 0.02894 | 0.05145 | 0.00643 | 0.17042 | 0.01929 | 0.21222 |
| HHATL | NM_020707 | 0.00000 | 0.00322 | 0.06431 | 0.16399 | 0.01929 | 0.24116 |
| TAAR8 | NM_053278 | 0.11576 | 0.07717 | 0.12540 | 0.01608 | 0.14791 | 0.01286 |
| ASB8 | NM_024095 | 0.07717 | 0.08039 | 0.10932 | 0.00965 | 0.20900 | 0.01286 |
| TTLL10 | NM_001130045 | 0.20579 | 0.04180 | 0.08039 | 0.05788 | 0.06431 | 0.04823 |
| ABCG4 | NM_022169 | 0.00965 | 0.11254 | 0.10932 | 0.03537 | 0.17042 | 0.06109 |
| BCL11B | NM_138576 | 0.03859 | 0.17685 | 0.08039 | 0.07717 | 0.06109 | 0.06431 |
| USP10 | NM_001272075 | 0.10289 | 0.06431 | 0.13505 | 0.02572 | 0.14791 | 0.02251 |
| TMEM182 | NM_144632 | 0.23794 | 0.09003 | 0.09325 | 0.01608 | 0.05145 | 0.01286 |
| VAMP2 | NM_014232 | 0.08360 | 0.07717 | 0.03859 | 0.12862 | 0.05466 | 0.11897 |
| ZNF135 | NM_001289401 | 0.08360 | 0.16720 | 0.09325 | 0.03537 | 0.06431 | 0.05788 |
| AP1S1 | NM_001283 | 0.06431 | 0.09003 | 0.09646 | 0.05788 | 0.08360 | 0.10932 |
| DSEL | NM_032160 | 0.00000 | 0.22508 | 0.00000 | 0.12862 | 0.00322 | 0.14791 |
| LDLRAD3 | NM_174902 | 0.27974 | 0.00322 | 0.06431 | 0.02894 | 0.10289 | 0.02572 |
| EIF5A | NM_001143760 | 0.13505 | 0.11254 | 0.06109 | 0.08039 | 0.03537 | 0.08360 |
| GNG11 | NM_004126 | 0.13826 | 0.02251 | 0.17685 | 0.04180 | 0.07074 | 0.05788 |
| MBNL3 | NM_133486 | 0.06752 | 0.09646 | 0.11254 | 0.06109 | 0.10289 | 0.06752 |
| UIMC1 | NM_001199297 | 0.36334 | 0.11576 | 0.01286 | 0.00965 | 0.00000 | 0.00965 |
| GCNT2 | NM_145649 | 0.03859 | 0.28617 | 0.03859 | 0.04823 | 0.08682 | 0.01608 |
| SHD | NM_020209 | 0.00965 | 0.23151 | 0.00000 | 0.16399 | 0.00000 | 0.10932 |
| SULT2B1 | NM_177973 | 0.09003 | 0.08039 | 0.17685 | 0.01608 | 0.11576 | 0.03537 |
| SRP54 | NM_003136 | 0.09003 | 0.07717 | 0.07395 | 0.09325 | 0.05466 | 0.12862 |
| ZNF605 | NM_001164715 | 0.00000 | 0.17363 | 0.00000 | 0.09325 | 0.00965 | 0.24116 |
| C9orf84 | NM_173521 | 0.02251 | 0.13505 | 0.03859 | 0.05466 | 0.24116 | 0.02572 |
| ADCYAP1 | NM_001099733 | 0.12540 | 0.14148 | 0.07074 | 0.07074 | 0.05466 | 0.05788 |
| ORIN1 | NM_012363 | 0.03537 | 0.07395 | 0.13505 | 0.07074 | 0.16077 | 0.04502 |
| SEPTIN9 | NM_001113496 | 0.09003 | 0.03859 | 0.19936 | 0.00965 | 0.17363 | 0.01286 |
| HAVCR1 | NM_001173393 | 0.07717 | 0.14148 | 0.09325 | 0.05788 | 0.10289 | 0.05466 |
| C3orf80 | NM_001168214 | 0.04502 | 0.09646 | 0.03859 | 0.18006 | 0.06109 | 0.10611 |
| TTC37 | NM_014639 | 0.00000 | 0.10289 | 0.00322 | 0.06431 | 0.01929 | 0.33762 |
| DDX43 | NM_018665 | 0.01286 | 0.02251 | 0.06752 | 0.10289 | 0.10932 | 0.21222 |
| ZNF277 | NM_021994 | 0.00000 | 0.34405 | 0.03537 | 0.04502 | 0.07074 | 0.03859 |
| AP3M1 | NM_207012 | 0.33441 | 0.02251 | 0.03859 | 0.03215 | 0.06752 | 0.03859 |
| TNFAIP8L2- | NM_001204848 | 0.19936 | 0.17042 | 0.08039 | 0.02572 | 0.04823 | 0.01608 |
| SCNM1 | | | | | | | |
| SMPDL3B | NM_001009568 | 0.03859 | 0.15756 | 0.11254 | 0.07717 | 0.06431 | 0.09003 |
| CORO1C | NM_001105237 | 0.13826 | 0.01929 | 0.15113 | 0.04502 | 0.12862 | 0.05788 |
| CDK2AP2 | NM_001271849 | 0.04180 | 0.03859 | 0.17042 | 0.00965 | 0.27331 | 0.00965 |
| RIMKLB | NM_020734 | 0.09003 | 0.00322 | 0.19293 | 0.00000 | 0.25402 | 0.00322 |
| TNS3 | NM_022748 | 0.44051 | 0.00643 | 0.06431 | 0.00965 | 0.01929 | 0.00322 |
| GATC | NM_176818 | 0.08360 | 0.14469 | 0.09646 | 0.08682 | 0.10289 | 0.02894 |
| SOCS3 | NM_003955 | 0.08360 | 0.07395 | 0.12540 | 0.13183 | 0.07717 | 0.05466 |
| C10orf105 | NM_001164375 | 0.03537 | 0.16077 | 0.12540 | 0.03215 | 0.10932 | 0.08360 |
| SSH3 | NM_017857 | 0.09003 | 0.14791 | 0.02572 | 0.06431 | 0.03537 | 0.18650 |
| LSP1 | NM_001289005 | 0.00000 | 0.22186 | 0.00000 | 0.19936 | 0.02251 | 0.10932 |
| YPEL5 | NM_001127400 | 0.25080 | 0.07717 | 0.04823 | 0.05788 | 0.06431 | 0.05466 |
| OBP2A | NM_014582 | 0.11576 | 0.14148 | 0.07395 | 0.09325 | 0.04180 | 0.09003 |
| TRIM5 | NM_033092 | 0.11576 | 0.02894 | 0.15434 | 0.03537 | 0.20900 | 0.01286 |
| SMIM24 | NM_001136503 | 0.02572 | 0.13826 | 0.04180 | 0.15756 | 0.09325 | 0.09968 |
| IL1RL2 | NM_003854 | 0.14148 | 0.09646 | 0.09325 | 0.09325 | 0.06431 | 0.06752 |
| KIF13A | NM_001105568 | 0.15434 | 0.04180 | 0.08039 | 0.03537 | 0.12862 | 0.11897 |
| IRX1 | NM_024337 | 0.00965 | 0.00643 | 0.17363 | 0.03537 | 0.28296 | 0.05145 |
| CD81 | NM_004356 | 0.04823 | 0.15756 | 0.10289 | 0.02894 | 0.17363 | 0.04823 |
| AHCYL1 | NM_001242674 | 0.09003 | 0.24116 | 0.03859 | 0.06431 | 0.03859 | 0.09003 |
| GPR26 | NM_153442 | 0.30547 | 0.19614 | 0.03859 | 0.00965 | 0.00322 | 0.00965 |
| RPL6 | NM_000970 | 0.14791 | 0.00322 | 0.25402 | 0.00965 | 0.14148 | 0.00643 |
| OR4C11 | NM_001004700 | 0.13505 | 0.05145 | 0.13505 | 0.08360 | 0.06431 | 0.09325 |
| ZNF408 | NM_001184751 | 0.08360 | 0.07717 | 0.07074 | 0.15756 | 0.06752 | 0.10611 |
| SLC26A8 | NM_001193476 | 0.25402 | 0.00643 | 0.08039 | 0.07717 | 0.04180 | 0.10289 |
| FMO3 | NM_006894 | 0.06752 | 0.06752 | 0.16077 | 0.03215 | 0.22186 | 0.01286 |
| HNRNPR | NM_001102398 | 0.09325 | 0.06431 | 0.18971 | 0.02894 | 0.17363 | 0.01608 |
| TPRN | NM_001128228 | 0.10289 | 0.02894 | 0.12219 | 0.04502 | 0.15113 | 0.11576 |
| CACNA2D2 | NM_001174051 | 0.02572 | 0.14148 | 0.09325 | 0.05788 | 0.13826 | 0.10932 |
| EFHC1 | NM_001172420 | 0.06752 | 0.00322 | 0.18971 | 0.00965 | 0.28939 | 0.00965 |
| TEC | NM_003215 | 0.01929 | 0.07395 | 0.13505 | 0.04502 | 0.24437 | 0.05145 |
| MSRB1 | NM_016332 | 0.07717 | 0.23151 | 0.10611 | 0.03215 | 0.06431 | 0.06109 |
| ZNF248 | NM_001267606 | 0.03859 | 0.03215 | 0.15113 | 0.03859 | 0.24116 | 0.07395 |
| PELP1 | NM_014389 | 0.29260 | 0.07717 | 0.10611 | 0.00965 | 0.07717 | 0.01286 |
| PROC | NM_000312 | 0.13826 | 0.17042 | 0.07074 | 0.07717 | 0.06109 | 0.05788 |
| NPB | NM_148896 | 0.11576 | 0.13505 | 0.12540 | 0.02572 | 0.14791 | 0.02572 |
| LTF | NM_001199149 | 0.00322 | 0.18006 | 0.03859 | 0.15756 | 0.05466 | 0.14791 |
| FBXO33 | NM_203301 | 0.09003 | 0.08682 | 0.09968 | 0.06752 | 0.14791 | 0.09003 |
| ZEB1 | NM_001174096 | 0.24437 | 0.13183 | 0.04180 | 0.06431 | 0.01929 | 0.08039 |
| MCL1 | NM_021960 | 0.00000 | 0.20257 | 0.00000 | 0.17685 | 0.02894 | 0.17685 |
| FOPNL | NM_144600 | 0.09968 | 0.30547 | 0.03859 | 0.07074 | 0.01286 | 0.05788 |
| UBE2L3 | NM_001256355 | 0.04502 | 0.00643 | 0.09325 | 0.11254 | 0.16077 | 0.17042 |
| SMARCA2 | NM_001289397 | 0.01286 | 0.00643 | 0.00000 | 0.17363 | 0.01608 | 0.37942 |
| TRIM62 | NM_018207 | 0.02894 | 0.14148 | 0.07717 | 0.09968 | 0.05466 | 0.18650 |
| UBXN7 | NM_015562 | 0.04502 | 0.16077 | 0.19614 | 0.01286 | 0.16077 | 0.01608 |
| ATF7IP | NM_018179 | 0.04180 | 0.07717 | 0.04180 | 0.11897 | 0.03537 | 0.27653 |
| ABI1 | NM_001178116 | 0.08360 | 0.08682 | 0.11254 | 0.07074 | 0.13826 | 0.09968 |
| EEFSEC | NM_021937 | 0.09325 | 0.05145 | 0.14469 | 0.04823 | 0.20257 | 0.05466 |
| USP7 | NM_003470 | 0.07717 | 0.20579 | 0.08682 | 0.03215 | 0.14148 | 0.05145 |
| TMPRSS9 | NM_182973 | 0.16399 | 0.05466 | 0.09003 | 0.13505 | 0.07074 | 0.08039 |
| C9orf92 | NM_001271829 | 0.22830 | 0.00643 | 0.19293 | 0.00965 | 0.14791 | 0.01286 |
| SAMD8 | NM_144660 | 0.21865 | 0.01286 | 0.10611 | 0.05788 | 0.11576 | 0.08682 |
| ZNF766 | NM_001010851 | 0.12540 | 0.00965 | 0.19614 | 0.00965 | 0.25402 | 0.00643 |
| DNAH5 | NM_001369 | 0.12540 | 0.03215 | 0.19293 | 0.03537 | 0.18971 | 0.02572 |
| GPS2 | NM_004489 | 0.10289 | 0.19614 | 0.07074 | 0.07395 | 0.04823 | 0.10932 |
| NFIC | NM_205843 | 0.16399 | 0.00965 | 0.06752 | 0.11254 | 0.06109 | 0.18650 |
| TERF2 | NM_005652 | 0.41479 | 0.02894 | 0.09325 | 0.00965 | 0.04823 | 0.00965 |
| HNRNPF | NM_004966 | 0.15113 | 0.03537 | 0.20579 | 0.03215 | 0.11576 | 0.06431 |
| NPFF | NM_003717 | 0.02572 | 0.11576 | 0.03859 | 0.17685 | 0.06109 | 0.18650 |
| CST4 | NM_001899 | 0.07074 | 0.00643 | 0.22830 | 0.00965 | 0.28296 | 0.00965 |
| FLOT2 | NM_004475 | 0.16720 | 0.04502 | 0.00000 | 0.18650 | 0.00000 | 0.20900 |
| PPP3R2 | NM_147180 | 0.39871 | 0.02251 | 0.10611 | 0.00965 | 0.05466 | 0.01608 |
| PLA2G4B | NM_001114633 | 0.01929 | 0.20257 | 0.03859 | 0.13505 | 0.04823 | 0.16399 |
| PGAP2 | NM_001256235 | 0.06752 | 0.09646 | 0.08039 | 0.09646 | 0.13826 | 0.12862 |
| ADRA2A | NM_000681 | 0.08360 | 0.13183 | 0.09325 | 0.04502 | 0.14791 | 0.10932 |
| C7orf26 | NM_024067 | 0.03537 | 0.16720 | 0.11254 | 0.16077 | 0.06109 | 0.07395 |
| AMER1 | NM_152424 | 0.00000 | 0.29582 | 0.00000 | 0.13505 | 0.03215 | 0.14791 |
| EOMES | NM_005442 | 0.03537 | 0.04502 | 0.17685 | 0.07074 | 0.23794 | 0.04823 |
| CYB5D2 | NM_144611 | 0.08360 | 0.13183 | 0.09646 | 0.09968 | 0.09325 | 0.10932 |
| RCOR3 | NM_001136223 | 0.10289 | 0.20257 | 0.10932 | 0.02572 | 0.14791 | 0.02572 |
| TBP | NM_001172085 | 0.10289 | 0.04180 | 0.13505 | 0.02572 | 0.25723 | 0.05145 |
| SLURP1 | NM_020427 | 0.11576 | 0.05145 | 0.09003 | 0.15113 | 0.09325 | 0.11576 |
| PLAG1 | NM_001114635 | 0.01929 | 0.29904 | 0.00000 | 0.13826 | 0.00000 | 0.16077 |
| ELP3 | NM_018091 | 0.04180 | 0.29582 | 0.02572 | 0.09646 | 0.05788 | 0.09968 |
| CTDNEP1 | NM_001143775 | 0.02572 | 0.07717 | 0.03859 | 0.11897 | 0.15113 | 0.20900 |
| PPAT | NM_002703 | 0.31833 | 0.02894 | 0.08039 | 0.02894 | 0.15113 | 0.01286 |
| RAPGEF6 | NM_016340 | 0.18971 | 0.02894 | 0.18006 | 0.00965 | 0.20257 | 0.00965 |
| TOP3B | NM_001282113 | 0.09325 | 0.06109 | 0.08039 | 0.15434 | 0.06431 | 0.17042 |
| MTARC2 | NM_017898 | 0.03859 | 0.09003 | 0.02572 | 0.18971 | 0.05466 | 0.22508 |
| ITGB1BP2 | NM_012278 | 0.12540 | 0.18328 | 0.13183 | 0.04502 | 0.09003 | 0.05145 |
| PLAA | NM_001031689 | 0.01929 | 0.40514 | 0.02251 | 0.07717 | 0.05466 | 0.04823 |
| ATP7A | NM_000052 | 0.06752 | 0.13826 | 0.07074 | 0.13826 | 0.10289 | 0.10932 |
| GDA | NM_001242506 | 0.00643 | 0.45981 | 0.01286 | 0.04502 | 0.05466 | 0.05145 |
| MPHOSPH8 | NM_017520 | 0.12862 | 0.00643 | 0.19293 | 0.04502 | 0.23151 | 0.02572 |
| TLE3 | NM_020908 | 0.12540 | 0.00643 | 0.19936 | 0.00000 | 0.29904 | 0.00000 |
| ADH5 | NM_000671 | 0.06109 | 0.05788 | 0.10611 | 0.10932 | 0.13183 | 0.16399 |
| RPS15A | NM_001019 | 0.19936 | 0.03215 | 0.06752 | 0.10932 | 0.00643 | 0.21543 |
| SH2D4B | NM_001145719 | 0.09003 | 0.19293 | 0.03859 | 0.13826 | 0.06109 | 0.10932 |
| SMARCAD1 | NM_001254949 | 0.00322 | 0.14148 | 0.03859 | 0.17042 | 0.08682 | 0.19293 |
| ALAS1 | NM_199166 | 0.15434 | 0.00643 | 0.20900 | 0.03215 | 0.21865 | 0.01286 |
| ATP6V0D2 | NM_152565 | 0.03859 | 0.31833 | 0.04180 | 0.07074 | 0.05466 | 0.10932 |
| IL20RB | NM_144717 | 0.13505 | 0.03859 | 0.17363 | 0.00965 | 0.26367 | 0.01286 |
| SOCS2 | NM_001270467 | 0.39871 | 0.01929 | 0.08039 | 0.05145 | 0.04823 | 0.03859 |
| MBD3L2 | NM_144614 | 0.02572 | 0.02894 | 0.12540 | 0.05466 | 0.23794 | 0.16399 |
| MTX3 | NM_001167741 | 0.05466 | 0.07717 | 0.09325 | 0.14469 | 0.14791 | 0.11897 |
| HIST1H2BK | NM_080593 | 0.09325 | 0.14469 | 0.10611 | 0.03537 | 0.17363 | 0.08360 |
| CTBP1 | NM_001012614 | 0.10289 | 0.00322 | 0.22830 | 0.00000 | 0.29582 | 0.00643 |
| KBTBD7 | NM_032138 | 0.33441 | 0.08039 | 0.10611 | 0.03215 | 0.05788 | 0.02572 |
| DNPEP | NM_012100 | 0.00000 | 0.04502 | 0.00000 | 0.24759 | 0.00000 | 0.34727 |
| EIF2B2 | NM_014239 | 0.15756 | 0.18328 | 0.07395 | 0.05145 | 0.12540 | 0.04823 |
| LRIT1 | NM_015613 | 0.17685 | 0.13183 | 0.12540 | 0.06431 | 0.10289 | 0.03859 |
| PACRG | NM_001080378 | 0.02894 | 0.33441 | 0.04180 | 0.11254 | 0.05145 | 0.07074 |
| LARP6 | NM_001286679 | 0.29260 | 0.14148 | 0.08039 | 0.04502 | 0.05466 | 0.02572 |
| MOB4 | NM_015387 | 0.27653 | 0.01286 | 0.15113 | 0.02894 | 0.14791 | 0.02251 |
| HAUS4 | NM_001166269 | 0.32476 | 0.00643 | 0.16399 | 0.03215 | 0.06752 | 0.04823 |
| RALGDS | NM_001271774 | 0.39871 | 0.03859 | 0.02572 | 0.06431 | 0.04823 | 0.06752 |
| ENY2 | NM_001193557 | 0.03215 | 0.03859 | 0.19293 | 0.07395 | 0.20257 | 0.10289 |
| HSD17B1 | NM_000413 | 0.11576 | 0.03859 | 0.29904 | 0.05466 | 0.07717 | 0.05788 |
| PAAF1 | NM_001267803 | 0.06752 | 0.05466 | 0.04823 | 0.17363 | 0.05466 | 0.24759 |
| THOP1 | NM_003249 | 0.23794 | 0.13183 | 0.17685 | 0.00965 | 0.08039 | 0.00965 |
| EXOC3L2 | NM_138568 | 0.09325 | 0.07395 | 0.09968 | 0.03859 | 0.12862 | 0.21222 |
| PRDX1 | NM_001202431 | 0.00000 | 0.22508 | 0.00000 | 0.17363 | 0.03537 | 0.21222 |
| BACE1 | NM_138971 | 0.09325 | 0.23473 | 0.09646 | 0.02572 | 0.16077 | 0.03859 |
| DYNC1I1 | NM_001135556 | 0.37942 | 0.08039 | 0.09968 | 0.02894 | 0.03859 | 0.02251 |
| FAM178B | NM_001172667 | 0.06752 | 0.01929 | 0.25080 | 0.03215 | 0.24437 | 0.03537 |
| PHKB | NM_000293 | 0.03859 | 0.41479 | 0.01286 | 0.07395 | 0.04502 | 0.06752 |
| TCF7L2 | NM_001198529 | 0.00965 | 0.38264 | 0.03859 | 0.10611 | 0.01608 | 0.09968 |
| CSAG1 | NM_153478 | 0.07074 | 0.29260 | 0.11254 | 0.05788 | 0.09325 | 0.02572 |
| AKAP6 | NM_004274 | 0.18650 | 0.24759 | 0.09646 | 0.05466 | 0.05466 | 0.01608 |
| POLD3 | NM_006591 | 0.00965 | 0.23473 | 0.03859 | 0.17042 | 0.05466 | 0.14791 |
| PCDHGB1 | NM_018922 | 0.20579 | 0.06752 | 0.10611 | 0.06752 | 0.16077 | 0.05145 |
| COL9A3 | NM_001853 | 0.00000 | 0.06109 | 0.09968 | 0.07074 | 0.32154 | 0.10611 |
| PMM1 | NM_002676 | 0.06431 | 0.03859 | 0.17685 | 0.06752 | 0.24437 | 0.06752 |
| TTLL3 | NM_001025930 | 0.08360 | 0.14148 | 0.09325 | 0.17042 | 0.04823 | 0.12540 |
| C6orf48 | NM_001287484 | 0.16720 | 0.07717 | 0.16399 | 0.03537 | 0.15113 | 0.06752 |
| SHCBP1 | NM_024745 | 0.03859 | 0.07717 | 0.15113 | 0.07717 | 0.15756 | 0.16077 |
| PLEKHM3 | NM_001080475 | 0.16077 | 0.02894 | 0.17042 | 0.02572 | 0.24116 | 0.03537 |
| BTK | NM_000061 | 0.10932 | 0.07717 | 0.08360 | 0.15756 | 0.07717 | 0.16077 |
| YES1 | NM_005433 | 0.00000 | 0.09003 | 0.01286 | 0.25402 | 0.05466 | 0.25402 |
| EIF4EBP1 | NM_004095 | 0.06752 | 0.08039 | 0.16720 | 0.07717 | 0.10289 | 0.17042 |
| DMPK | NM_001081563 | 0.17363 | 0.00643 | 0.12219 | 0.06752 | 0.06109 | 0.23473 |
| LMO2 | NM_001142316 | 0.18650 | 0.17363 | 0.05466 | 0.13826 | 0.05466 | 0.05788 |
| GHRHR | NM_000823 | 0.04823 | 0.06752 | 0.03859 | 0.25402 | 0.04823 | 0.21222 |
| ABHD8 | NM_024527 | 0.01929 | 0.14148 | 0.03859 | 0.22186 | 0.05466 | 0.19293 |
| RCN2 | NM_002902 | 0.04180 | 0.08039 | 0.03859 | 0.21222 | 0.00322 | 0.29260 |
| KIAA0247 | NM_014734 | 0.09968 | 0.01286 | 0.36334 | 0.00965 | 0.17363 | 0.01286 |
| EPC1 | NM_001282391 | 0.00965 | 0.30547 | 0.11254 | 0.10932 | 0.06109 | 0.07395 |
| VSIG10L | NM_001163922 | 0.09968 | 0.02894 | 0.09325 | 0.16399 | 0.08039 | 0.20900 |
| NBR1 | NM_001291571 | 0.01286 | 0.09003 | 0.03859 | 0.17685 | 0.05466 | 0.30225 |
| LZTR1 | NM_006767 | 0.15434 | 0.29260 | 0.07717 | 0.04502 | 0.05466 | 0.05145 |
| CA7 | NM_005182 | 0.02572 | 0.19614 | 0.00000 | 0.18971 | 0.00965 | 0.25402 |
| ACOT7 | NM_181864 | 0.02572 | 0.11897 | 0.08039 | 0.17042 | 0.06109 | 0.21865 |
| ATP2C1 | NM_001199181 | 0.13505 | 0.07717 | 0.08682 | 0.15434 | 0.10289 | 0.11897 |
| CD274 | NM_014143 | 0.07717 | 0.00643 | 0.18971 | 0.02572 | 0.36334 | 0.01608 |
| LEMD2 | NM_181336 | 0.46624 | 0.03215 | 0.01286 | 0.04502 | 0.00000 | 0.12219 |
| CLDN19 | NM_001185117 | 0.14469 | 0.11576 | 0.15113 | 0.05788 | 0.17363 | 0.03537 |
| ZNF728 | NM_001267716 | 0.29904 | 0.18006 | 0.00000 | 0.07395 | 0.00000 | 0.12862 |
| IHH | NM_002181 | 0.03859 | 0.35691 | 0.07395 | 0.05788 | 0.10289 | 0.05145 |
| RREB1 | NM_001003699 | 0.08360 | 0.02572 | 0.19936 | 0.04502 | 0.25402 | 0.07717 |
| CLCN3 | NM_001243374 | 0.04823 | 0.30547 | 0.08039 | 0.13505 | 0.06431 | 0.05145 |
| RNF150 | NM_020724 | 0.32476 | 0.15434 | 0.03859 | 0.05788 | 0.03537 | 0.07395 |
| CDC27 | NM_001114091 | 0.04823 | 0.07717 | 0.11254 | 0.07074 | 0.32154 | 0.05788 |
| IFIT3 | NM_001549 | 0.02894 | 0.29260 | 0.04502 | 0.15756 | 0.04823 | 0.11576 |
| POU2F3 | NM_014352 | 0.12219 | 0.14791 | 0.12219 | 0.07395 | 0.15113 | 0.07074 |
| KCNMA1 | NM_001271518 | 0.18971 | 0.03215 | 0.17363 | 0.05788 | 0.14791 | 0.08682 |
| HUS1B | NM_148959 | 0.16720 | 0.03215 | 0.28296 | 0.00965 | 0.18650 | 0.01286 |
| WARS2 | NM_015836 | 0.00000 | 0.13183 | 0.00000 | 0.17685 | 0.00000 | 0.38264 |
| KRT18 | NM_199187 | 0.07074 | 0.22186 | 0.18971 | 0.03537 | 0.15756 | 0.01608 |
| FAM217B | NM_001190826 | 0.13505 | 0.17042 | 0.08039 | 0.13826 | 0.05466 | 0.11254 |
| LYPD5 | NM_001288763 | 0.29260 | 0.16077 | 0.12540 | 0.03537 | 0.05466 | 0.02251 |
| CHMP2B | NM_014043 | 0.00965 | 0.29260 | 0.03859 | 0.16720 | 0.06431 | 0.11897 |
| AICDA | NM_020661 | 0.03537 | 0.22508 | 0.03859 | 0.17685 | 0.03537 | 0.18006 |
| EDARADD | NM_080738 | 0.07074 | 0.09003 | 0.24116 | 0.05788 | 0.21865 | 0.01608 |
| ZNF239 | NM_005674 | 0.15434 | 0.03859 | 0.17685 | 0.04823 | 0.20257 | 0.07395 |
| COQ6 | NM_182480 | 0.25080 | 0.07717 | 0.16077 | 0.00643 | 0.18971 | 0.00965 |
| RFPL2 | NM_001098527 | 0.02572 | 0.19614 | 0.11254 | 0.13826 | 0.14791 | 0.07395 |
| CCNI | NM_006835 | 0.06431 | 0.14148 | 0.10611 | 0.13183 | 0.15113 | 0.09968 |
| SPEG | NM_001173476 | 0.12540 | 0.29260 | 0.14148 | 0.01608 | 0.10932 | 0.01286 |
| FBXO15 | NM_001142958 | 0.28939 | 0.00000 | 0.13183 | 0.00965 | 0.25402 | 0.01286 |
| PHF3 | NM_001290260 | 0.12219 | 0.09003 | 0.08360 | 0.15756 | 0.10289 | 0.14148 |
| OR2C3 | NM_198074 | 0.00965 | 0.29904 | 0.00643 | 0.22186 | 0.01286 | 0.14791 |
| GNB4 | NM_021629 | 0.03859 | 0.03859 | 0.22508 | 0.03537 | 0.30868 | 0.05145 |
| HIST1H3G | NM_003534 | 0.08360 | 0.14148 | 0.18006 | 0.03215 | 0.23473 | 0.02572 |
| FXN | NM_001161706 | 0.06752 | 0.07717 | 0.08039 | 0.17685 | 0.04823 | 0.25080 |
| PIANP | NM_001244015 | 0.08682 | 0.11576 | 0.00000 | 0.24116 | 0.00000 | 0.26045 |
| PIGZ | NM_025163 | 0.19936 | 0.02251 | 0.27974 | 0.00965 | 0.18650 | 0.00643 |
| ANKRD45 | NM_198493 | 0.08682 | 0.12219 | 0.18006 | 0.00965 | 0.29904 | 0.00643 |
| MRPS15 | NM_031280 | 0.13826 | 0.17363 | 0.07074 | 0.12219 | 0.09003 | 0.10932 |
| SLC39A1 | NM_001271960 | 0.26688 | 0.16399 | 0.07395 | 0.06752 | 0.06431 | 0.07074 |
| KAZALD1 | NM_030929 | 0.06431 | 0.14148 | 0.03859 | 0.17363 | 0.05466 | 0.23473 |
| DDX1 | NM_004939 | 0.09003 | 0.08682 | 0.19614 | 0.06431 | 0.22508 | 0.04823 |
| WNT2B | NM_004185 | 0.09003 | 0.03859 | 0.10932 | 0.24437 | 0.10289 | 0.12862 |
| CTXN3 | NM_001048252 | 0.08360 | 0.09646 | 0.15113 | 0.03537 | 0.30868 | 0.03859 |
| GSN | NM_001258030 | 0.33762 | 0.05145 | 0.09646 | 0.06752 | 0.07074 | 0.09003 |
| USP22 | NM_015276 | 0.38907 | 0.14148 | 0.08682 | 0.02572 | 0.04823 | 0.02251 |
| LGALS9B | NM_001042685 | 0.04180 | 0.06109 | 0.18971 | 0.08682 | 0.22508 | 0.10932 |
| MSRB3 | NM_001193460 | 0.03859 | 0.34405 | 0.09325 | 0.08039 | 0.08682 | 0.07395 |
| ZNF354A | NM_005649 | 0.16720 | 0.15756 | 0.16399 | 0.02572 | 0.18650 | 0.01608 |
| TMEM184C | NM_018241 | 0.03859 | 0.05145 | 0.18971 | 0.11254 | 0.19614 | 0.12862 |
| PHF19 | NM_001286840 | 0.27653 | 0.02251 | 0.09646 | 0.05466 | 0.18650 | 0.08039 |
| P2RX1 | NM_002558 | 0.12540 | 0.17042 | 0.06752 | 0.06752 | 0.18971 | 0.09646 |
| CORO6 | NM_032854 | 0.07074 | 0.13183 | 0.05788 | 0.17363 | 0.05466 | 0.22830 |
| SLC7A6 | NM_001076785 | 0.12862 | 0.03859 | 0.17685 | 0.07395 | 0.18971 | 0.11254 |
| NDRG4 | NM_022910 | 0.00322 | 0.30225 | 0.03859 | 0.15756 | 0.05788 | 0.16077 |
| SLC16A3 | NM_004207 | 0.16399 | 0.17042 | 0.12540 | 0.06431 | 0.13826 | 0.05788 |
| MOSPD1 | NM_019556 | 0.03859 | 0.03215 | 0.05788 | 0.27653 | 0.06752 | 0.24759 |
| ASB4 | NM_016116 | 0.25402 | 0.02894 | 0.18006 | 0.04823 | 0.15113 | 0.05788 |
| PPM1G | NM_177983 | 0.05466 | 0.24759 | 0.04502 | 0.18328 | 0.05145 | 0.14148 |
| SERPINB8 | NM_001276490 | 0.29260 | 0.00322 | 0.20579 | 0.01286 | 0.18650 | 0.02251 |
| MROH6 | NM_001100878 | 0.14469 | 0.22186 | 0.12219 | 0.12862 | 0.06431 | 0.04180 |
| LTBP1 | NM_001166266 | 0.15756 | 0.00643 | 0.17363 | 0.07395 | 0.09646 | 0.21543 |
| SLCO5A1 | NM_001146008 | 0.02251 | 0.02894 | 0.09325 | 0.20257 | 0.23794 | 0.14148 |
| SOSTDC1 | NM_015464 | 0.12862 | 0.00322 | 0.25402 | 0.02572 | 0.27974 | 0.03537 |
| TVP23B | NM_016078 | 0.00965 | 0.08039 | 0.00000 | 0.27974 | 0.01929 | 0.33762 |
| IQCF2 | NM_203424 | 0.14791 | 0.13183 | 0.19293 | 0.05788 | 0.14791 | 0.04823 |
| CES3 | NM_001185176 | 0.10289 | 0.22508 | 0.13505 | 0.05788 | 0.11576 | 0.09003 |
| POU4F1 | NM_006237 | 0.31190 | 0.17042 | 0.09646 | 0.03859 | 0.09968 | 0.01286 |
| CCNT2 | NM_058241 | 0.15756 | 0.03215 | 0.15113 | 0.06752 | 0.20900 | 0.11254 |
| NDUFA5 | NM_001282419 | 0.03859 | 0.24759 | 0.02572 | 0.23473 | 0.07717 | 0.10611 |
| ZNF443 | NM_005815 | 0.27653 | 0.14148 | 0.07717 | 0.06752 | 0.06109 | 0.10611 |
| OCLN | NM_001205255 | 0.00000 | 0.13183 | 0.08039 | 0.19614 | 0.05145 | 0.27331 |
| PPP1R37 | NM_019121 | 0.03537 | 0.39871 | 0.03859 | 0.09325 | 0.05466 | 0.11254 |
| NKX2-1 | NM_003317 | 0.01286 | 0.07717 | 0.27653 | 0.07395 | 0.22186 | 0.07074 |
| ZNF283 | NM_181845 | 0.19614 | 0.19614 | 0.17685 | 0.02894 | 0.08039 | 0.05466 |
| PCDH1 | NM_001278613 | 0.15113 | 0.08682 | 0.19936 | 0.03215 | 0.25402 | 0.01286 |
| DCP1A | NM_001290204 | 0.00322 | 0.16077 | 0.00000 | 0.27974 | 0.00000 | 0.29260 |
| MXRA8 | NM_001282582 | 0.25723 | 0.06752 | 0.12219 | 0.11897 | 0.07074 | 0.09968 |
| INA | NM_032727 | 0.01929 | 0.18650 | 0.00000 | 0.22186 | 0.00000 | 0.30868 |
| CPEB1 | NM_001079533 | 0.17685 | 0.21543 | 0.09646 | 0.06431 | 0.12540 | 0.05788 |
| OR2D3 | NM_001004684 | 0.04823 | 0.24759 | 0.03537 | 0.10932 | 0.05466 | 0.24437 |
| KIAA1109 | NM_015312 | 0.00965 | 0.14148 | 0.07074 | 0.15434 | 0.23151 | 0.13183 |
| DPM3 | NM_153741 | 0.32476 | 0.02894 | 0.17685 | 0.00965 | 0.18971 | 0.00965 |
| ARL1 | NM_001177 | 0.03537 | 0.18006 | 0.07717 | 0.19293 | 0.06752 | 0.18650 |
| TMTC2 | NM_152588 | 0.06431 | 0.11576 | 0.08039 | 0.16720 | 0.06109 | 0.25080 |
| DAZAP2 | NM_001136269 | 0.18971 | 0.17042 | 0.06431 | 0.15756 | 0.09325 | 0.06752 |
| ART1 | NM_004314 | 0.28939 | 0.00643 | 0.17685 | 0.05466 | 0.09325 | 0.12219 |
| NMT2 | NM_004808 | 0.00643 | 0.22508 | 0.00643 | 0.27653 | 0.01608 | 0.21222 |
| TIMM23B | NM_001290117 | 0.01286 | 0.04502 | 0.03859 | 0.25402 | 0.09968 | 0.29260 |
| TDRD9 | NM_153046 | 0.08360 | 0.33441 | 0.09968 | 0.06752 | 0.04823 | 0.10932 |
| PTDSS2 | NM_030783 | 0.03537 | 0.17042 | 0.01286 | 0.23473 | 0.04823 | 0.24437 |
| ZNF764 | NM_001172679 | 0.32476 | 0.02894 | 0.19293 | 0.03537 | 0.11576 | 0.04823 |
| DEFB127 | NM_139074 | 0.11576 | 0.34405 | 0.03859 | 0.07074 | 0.05466 | 0.12219 |
| CNTN1 | NM_001256064 | 0.15434 | 0.07717 | 0.18971 | 0.06752 | 0.19293 | 0.06431 |
| PRKRA | NM_001139518 | 0.52733 | 0.03859 | 0.10611 | 0.00965 | 0.05466 | 0.01286 |
| HMMR | NM_012484 | 0.40836 | 0.00643 | 0.20900 | 0.00965 | 0.10289 | 0.01286 |
| ROPN1 | NM_017578 | 0.01929 | 0.19614 | 0.00965 | 0.24437 | 0.04180 | 0.23794 |
| MCF2L | NM_001112732 | 0.06431 | 0.03215 | 0.15113 | 0.09646 | 0.15756 | 0.24759 |
| NCDN | NM_001014839 | 0.02572 | 0.27010 | 0.08039 | 0.16720 | 0.14148 | 0.06752 |
| USP2 | NM_001243759 | 0.14791 | 0.09646 | 0.19936 | 0.04823 | 0.19293 | 0.06752 |
| KCNK2 | NM_001017424 | 0.02894 | 0.37299 | 0.03859 | 0.13826 | 0.05466 | 0.11897 |
| ATP6V0C | NM_001694 | 0.03859 | 0.00322 | 0.25723 | 0.06431 | 0.28296 | 0.10611 |
| SPTBN4 | NM_025213 | 0.02251 | 0.34405 | 0.02572 | 0.17685 | 0.06431 | 0.11897 |
| ITGB4 | NM_001005619 | 0.03859 | 0.13826 | 0.11897 | 0.08682 | 0.24437 | 0.12862 |
| RNF24 | NM_001134338 | 0.09003 | 0.00643 | 0.23473 | 0.03215 | 0.33441 | 0.05788 |
| NLGN4Y | NM_014893 | 0.32476 | 0.14148 | 0.04180 | 0.11254 | 0.03537 | 0.10289 |
| TRIM73 | NM_198924 | 0.09003 | 0.16077 | 0.12540 | 0.13826 | 0.10289 | 0.14148 |
| ELP2 | NM_001242875 | 0.01929 | 0.16720 | 0.09646 | 0.16720 | 0.17363 | 0.13505 |
| YTHDF1 | NM_017798 | 0.00965 | 0.48232 | 0.07395 | 0.06752 | 0.06109 | 0.06431 |
| CHD4 | NM_001273 | 0.42444 | 0.02251 | 0.07717 | 0.06752 | 0.05145 | 0.11897 |
| SOCS1 | NM_003745 | 0.11576 | 0.09646 | 0.19614 | 0.05788 | 0.24116 | 0.05466 |
| C20orf196 | NM_152504 | 0.30547 | 0.03859 | 0.16399 | 0.05145 | 0.13826 | 0.06431 |
| NMNAT2 | NM_170706 | 0.28296 | 0.04502 | 0.09646 | 0.10611 | 0.10932 | 0.12219 |
| AWAT2 | NM_001002254 | 0.08682 | 0.03215 | 0.20579 | 0.01608 | 0.33441 | 0.09003 |
| SLITRK1 | NM_052910 | 0.08682 | 0.35370 | 0.18971 | 0.04823 | 0.07395 | 0.01286 |
| TBX1 | NM_080646 | 0.16077 | 0.07717 | 0.21543 | 0.01608 | 0.28617 | 0.00965 |
| SLC28A3 | NM_022127 | 0.04502 | 0.03859 | 0.12219 | 0.17042 | 0.13826 | 0.25080 |
| NRG3 | NM_001165973 | 0.02894 | 0.24759 | 0.10611 | 0.22186 | 0.07717 | 0.08682 |
| RHOB | NM_004040 | 0.42765 | 0.08039 | 0.15113 | 0.03537 | 0.03859 | 0.03537 |
| PPCS | NM_001287506 | 0.29260 | 0.07717 | 0.13505 | 0.07717 | 0.09646 | 0.09003 |
| INHBC | NM_005538 | 0.00000 | 0.17042 | 0.03859 | 0.24116 | 0.05466 | 0.26688 |
| TOE1 | NM_025077 | 0.10289 | 0.01286 | 0.19293 | 0.12862 | 0.16720 | 0.16720 |
| LDHA | NM_001165414 | 0.12862 | 0.13183 | 0.17363 | 0.13505 | 0.12862 | 0.07395 |
| NAE1 | NM_003905 | 0.29260 | 0.05145 | 0.15434 | 0.05788 | 0.16077 | 0.05788 |
| COG4 | NM_015386 | 0.28617 | 0.04502 | 0.08039 | 0.04502 | 0.24759 | 0.07395 |
| DAO | NM_001917 | 0.25402 | 0.23473 | 0.03859 | 0.07074 | 0.05145 | 0.12862 |
| RNASEH2B | NM_024570 | 0.27974 | 0.06752 | 0.19293 | 0.03537 | 0.18650 | 0.01608 |
| NTRK3 | NM_002530 | 0.24437 | 0.22508 | 0.12540 | 0.03215 | 0.13826 | 0.01286 |
| TLX2 | NM_016170 | 0.04502 | 0.24759 | 0.15434 | 0.03215 | 0.23794 | 0.06431 |
| HTR5A | NM_024012 | 0.12862 | 0.01929 | 0.20579 | 0.04502 | 0.24437 | 0.13826 |
| GJB3 | NM_001005752 | 0.18971 | 0.02251 | 0.09325 | 0.15756 | 0.10932 | 0.21222 |
| CAV1 | NM_001172896 | 0.15756 | 0.17363 | 0.09325 | 0.18971 | 0.07074 | 0.09968 |
| ELMO1 | NM_014800 | 0.27653 | 0.14148 | 0.10611 | 0.12219 | 0.01929 | 0.11897 |
| LMO3 | NM_001243610 | 0.13505 | 0.03215 | 0.19293 | 0.05466 | 0.28296 | 0.09003 |
| EXOSC1 | NM_016046 | 0.11576 | 0.17042 | 0.13505 | 0.07074 | 0.16720 | 0.12862 |
| PRDM8 | NM_020226 | 0.05466 | 0.32154 | 0.03859 | 0.06752 | 0.05466 | 0.25080 |
| EPG5 | NM_020964 | 0.04180 | 0.29260 | 0.04502 | 0.15756 | 0.10289 | 0.14791 |
| ARSH | NM_001011719 | 0.03859 | 0.25723 | 0.15434 | 0.07717 | 0.18650 | 0.07395 |
| DEDD | NM_001039711 | 0.03859 | 0.20257 | 0.03859 | 0.25723 | 0.03859 | 0.21222 |
| DUSP22 | NM_020185 | 0.10289 | 0.06431 | 0.13183 | 0.07395 | 0.18971 | 0.22508 |
| KCNH2 | NM_172056 | 0.02251 | 0.13826 | 0.04502 | 0.28939 | 0.07717 | 0.21543 |
| TOX2 | NM_001098797 | 0.11576 | 0.08682 | 0.08039 | 0.10611 | 0.28296 | 0.11897 |
| PCDH11X | NM_001168360 | 0.00965 | 0.20257 | 0.01286 | 0.25402 | 0.06109 | 0.25080 |
| SPEN | NM_015001 | 0.00000 | 0.22508 | 0.03859 | 0.22186 | 0.05466 | 0.25080 |
| NSD1 | NM_172349 | 0.02894 | 0.31190 | 0.12219 | 0.10932 | 0.13826 | 0.08039 |
| EGR1 | NM_001964 | 0.02572 | 0.33119 | 0.03859 | 0.17685 | 0.02572 | 0.19293 |
| C16orf72 | NM_014117 | 0.06431 | 0.26367 | 0.08360 | 0.17685 | 0.10289 | 0.09968 |
| TBCCD1 | NM_018138 | 0.10289 | 0.27010 | 0.14148 | 0.06431 | 0.07395 | 0.13826 |
| WDR54 | NM_032118 | 0.01929 | 0.11897 | 0.02251 | 0.26045 | 0.08039 | 0.28939 |
| TENM3 | NM_001080477 | 0.01929 | 0.17363 | 0.07074 | 0.13505 | 0.13826 | 0.25402 |
| PIP4K2B | NM_003559 | 0.16720 | 0.32154 | 0.15434 | 0.03537 | 0.10289 | 0.01286 |
| LIN7C | NM_018362 | 0.44051 | 0.03859 | 0.10611 | 0.04502 | 0.05466 | 0.10932 |
| PCDP1 | NM_001271049 | 0.19293 | 0.24759 | 0.12540 | 0.05788 | 0.07074 | 0.09968 |
| NDUFS6 | NM_004553 | 0.14148 | 0.32154 | 0.08360 | 0.07074 | 0.10289 | 0.07395 |
| IQCA1 | NM_001270585 | 0.03859 | 0.44373 | 0.07717 | 0.09646 | 0.06431 | 0.07395 |
| GAR1 | NM_032993 | 0.17685 | 0.33119 | 0.13183 | 0.06431 | 0.06431 | 0.02572 |
| ZNF274 | NM_133502 | 0.28617 | 0.09003 | 0.14469 | 0.01608 | 0.24759 | 0.01286 |
| CACNA1B | NM_001243812 | 0.15434 | 0.17042 | 0.15756 | 0.14469 | 0.12219 | 0.04823 |
| DPH5 | NM_015958 | 0.37299 | 0.07717 | 0.15434 | 0.03215 | 0.11576 | 0.04502 |
| IL18RAP | NM_003853 | 0.15756 | 0.35370 | 0.00322 | 0.16720 | 0.02894 | 0.09003 |
| KBTBD12 | NM_207335 | 0.09325 | 0.19614 | 0.18971 | 0.04502 | 0.24116 | 0.03537 |
| PNOC | NM_001284244 | 0.09003 | 0.02894 | 0.15434 | 0.25402 | 0.06431 | 0.20900 |
| NFE2L2 | NM_006164 | 0.27653 | 0.05788 | 0.18006 | 0.01608 | 0.24437 | 0.02572 |
| F13B | NM_001994 | 0.10289 | 0.08682 | 0.08039 | 0.18650 | 0.14148 | 0.20579 |
| EXO1 | NM_003686 | 0.38585 | 0.02251 | 0.13505 | 0.02894 | 0.18650 | 0.04502 |
| HEY1 | NM_001282851 | 0.17363 | 0.02894 | 0.19614 | 0.06752 | 0.27331 | 0.06431 |
| CBLC | NM_012116 | 0.14148 | 0.33441 | 0.00000 | 0.16720 | 0.06109 | 0.09968 |
| VWA2 | NM_001272046 | 0.08360 | 0.21865 | 0.17363 | 0.07717 | 0.16077 | 0.09325 |
| ALDH3B2 | NM_001031615 | 0.00643 | 0.29582 | 0.01286 | 0.17685 | 0.07074 | 0.24437 |
| ZNF81 | NM_007137 | 0.01929 | 0.22508 | 0.00000 | 0.27010 | 0.00000 | 0.29260 |
| C10orf82 | NM_144661 | 0.11576 | 0.09646 | 0.22508 | 0.15756 | 0.06431 | 0.14791 |
| ENAH | NM_018212 | 0.10289 | 0.05788 | 0.29260 | 0.03215 | 0.29904 | 0.02251 |
| OR51A7 | NM_001004749 | 0.35048 | 0.04180 | 0.19614 | 0.01608 | 0.16720 | 0.03859 |
| CKAP5 | NM_001008938 | 0.08682 | 0.16399 | 0.17685 | 0.07074 | 0.24437 | 0.06752 |
| PHKA1 | NM_001172436 | 0.00965 | 0.16077 | 0.00000 | 0.25402 | 0.01608 | 0.36977 |
| RAI14 | NM_001145520 | 0.09003 | 0.32797 | 0.03859 | 0.17685 | 0.05466 | 0.12219 |
| SBSN | NM_001166034 | 0.00643 | 0.16077 | 0.06752 | 0.23473 | 0.09646 | 0.24437 |
| SHCBP1L | NM_030933 | 0.12219 | 0.18650 | 0.11254 | 0.04502 | 0.26367 | 0.08039 |
| NUDT7 | NM_001243661 | 0.01929 | 0.50804 | 0.07717 | 0.06431 | 0.06752 | 0.07395 |
| KCNIP2 | NM_173193 | 0.11576 | 0.24116 | 0.15113 | 0.08360 | 0.10289 | 0.11897 |
| CCDC84 | NM_198489 | 0.41479 | 0.00322 | 0.18006 | 0.02894 | 0.16077 | 0.02572 |
| SMIM13 | NM_001135575 | 0.36334 | 0.00643 | 0.18971 | 0.00965 | 0.23794 | 0.00965 |
| OR2T33 | NM_001004695 | 0.05466 | 0.33119 | 0.12219 | 0.07395 | 0.16077 | 0.07395 |
| DPPA5 | NM_001025290 | 0.08039 | 0.04502 | 0.07074 | 0.27653 | 0.05466 | 0.28939 |
| VDAC1 | NM_003374 | 0.13826 | 0.13826 | 0.19614 | 0.04502 | 0.24437 | 0.05466 |
| DNTTIP2 | NM_014597 | 0.06431 | 0.08039 | 0.16077 | 0.10932 | 0.26367 | 0.13826 |
| LYPLA1 | NM_001279356 | 0.10932 | 0.22508 | 0.18971 | 0.04502 | 0.23794 | 0.01286 |
| DEFB125 | NM_153325 | 0.32154 | 0.03859 | 0.19614 | 0.07074 | 0.13183 | 0.06109 |
| TEAD4 | NM_003213 | 0.00000 | 0.34405 | 0.00322 | 0.18971 | 0.01929 | 0.26367 |
| QARS | NM_005051 | 0.00000 | 0.16077 | 0.03859 | 0.18650 | 0.14148 | 0.29260 |
| SGCB | NM_000232 | 0.00000 | 0.24759 | 0.00000 | 0.28296 | 0.00000 | 0.29260 |
| YBEY | NM_058181 | 0.12219 | 0.16077 | 0.06109 | 0.21543 | 0.05466 | 0.20900 |
| TUSC5 | NM_172367 | 0.09003 | 0.07717 | 0.19936 | 0.07395 | 0.31511 | 0.06752 |
| TUSC3 | NM_006765 | 0.11576 | 0.11897 | 0.22830 | 0.06431 | 0.25723 | 0.03859 |
| SP100 | NM_001206704 | 0.32476 | 0.01286 | 0.20900 | 0.02894 | 0.19293 | 0.05788 |
| ADCK3 | NM_020247 | 0.08360 | 0.16077 | 0.08039 | 0.03215 | 0.38264 | 0.08682 |
| TCP11L1 | NM_001145541 | 0.06752 | 0.22508 | 0.06752 | 0.18650 | 0.05466 | 0.22508 |
| PODN | NM_001199081 | 0.02251 | 0.29904 | 0.04180 | 0.22186 | 0.05466 | 0.18650 |
| UBE2E1 | NM_001202476 | 0.34405 | 0.01286 | 0.18006 | 0.06431 | 0.15113 | 0.07395 |
| ZNF627 | NM_145295 | 0.59164 | 0.01286 | 0.14148 | 0.02572 | 0.02894 | 0.02572 |
| SYT15 | NM_181519 | 0.13826 | 0.09646 | 0.19293 | 0.16399 | 0.05466 | 0.18006 |
| PRMT7 | NM_001290018 | 0.18650 | 0.18328 | 0.09646 | 0.11254 | 0.03537 | 0.21222 |
| TAF6 | NM_139315 | 0.48553 | 0.01286 | 0.17042 | 0.02572 | 0.12219 | 0.01286 |
| APRT | NM_000485 | 0.15434 | 0.13505 | 0.16720 | 0.09646 | 0.12862 | 0.14791 |
| TNPO2 | NM_001136195 | 0.42122 | 0.00643 | 0.22830 | 0.00000 | 0.17363 | 0.00000 |
| Clorf100 | NM_001276349 | 0.18328 | 0.33441 | 0.13505 | 0.06752 | 0.07074 | 0.03859 |
| TMEM179 | NM_001286389 | 0.01286 | 0.14469 | 0.03859 | 0.25402 | 0.06109 | 0.32154 |
| AGPAT4 | NM_020133 | 0.02572 | 0.14148 | 0.10611 | 0.16399 | 0.16720 | 0.22830 |
| PLXDC2 | NM_001282736 | 0.29582 | 0.23473 | 0.18006 | 0.02894 | 0.07074 | 0.02251 |
| LDB3 | NM_001171610 | 0.37299 | 0.19293 | 0.13183 | 0.07395 | 0.04823 | 0.01608 |
| IER3 | NM_003897 | 0.03859 | 0.09646 | 0.09325 | 0.27010 | 0.09968 | 0.24116 |
| OR2J2 | NM_030905 | 0.01929 | 0.03859 | 0.09646 | 0.16720 | 0.28296 | 0.23473 |
| Clorf162 | NM_174896 | 0.28296 | 0.05145 | 0.22830 | 0.02572 | 0.22508 | 0.02572 |
| HBD | NM_000519 | 0.12219 | 0.18328 | 0.07074 | 0.17685 | 0.14791 | 0.13826 |
| NKIRAS2 | NM_001144927 | 0.05466 | 0.24116 | 0.08039 | 0.18971 | 0.03859 | 0.23794 |
| ADAL | NM_001159280 | 0.06752 | 0.02251 | 0.15756 | 0.16720 | 0.24116 | 0.18650 |
| PLS3 | NM_001172335 | 0.02894 | 0.07717 | 0.10611 | 0.18650 | 0.24759 | 0.19614 |
| FAM151B | NM_205548 | 0.19293 | 0.17363 | 0.12219 | 0.18328 | 0.06109 | 0.10932 |
| SH3TC1 | NM_018986 | 0.04823 | 0.22508 | 0.03859 | 0.15756 | 0.05466 | 0.32154 |
| LRP10 | NM_014045 | 0.12540 | 0.07717 | 0.09646 | 0.18328 | 0.05466 | 0.30868 |
| WARS | NM_173701 | 0.09003 | 0.22508 | 0.10611 | 0.07717 | 0.16077 | 0.18650 |
| KRT79 | NM_175834 | 0.04823 | 0.07717 | 0.03859 | 0.30225 | 0.05788 | 0.32154 |
| PRR5 | NM_001017528 | 0.13826 | 0.39871 | 0.09325 | 0.06431 | 0.10289 | 0.04823 |
| SERF2 | NM_001199877 | 0.02572 | 0.29260 | 0.02572 | 0.25402 | 0.05466 | 0.19293 |
| C9orf57 | NM_001128618 | 0.18971 | 0.24759 | 0.05466 | 0.20579 | 0.05466 | 0.09646 |
| ZNF711 | NM_021998 | 0.00965 | 0.50161 | 0.00000 | 0.21543 | 0.00322 | 0.11897 |
| PLCG1 | NM_002660 | 0.02572 | 0.19614 | 0.15756 | 0.11254 | 0.28296 | 0.07395 |
| SFRP2 | NM_003013 | 0.25402 | 0.14148 | 0.24759 | 0.03215 | 0.15756 | 0.01608 |
| TXLNA | NM_175852 | 0.10932 | 0.02894 | 0.29582 | 0.02572 | 0.37621 | 0.01286 |
| PRAC1 | NM_032391 | 0.19293 | 0.20257 | 0.10611 | 0.03215 | 0.28939 | 0.02572 |
| CNOT10 | NM_001256742 | 0.09003 | 0.16720 | 0.13505 | 0.18328 | 0.14791 | 0.12862 |
| SRY | NM_003140 | 0.21865 | 0.00643 | 0.31190 | 0.04502 | 0.16077 | 0.10932 |
| MBD2 | NM_003927 | 0.18971 | 0.24759 | 0.18006 | 0.05145 | 0.16077 | 0.02251 |
| EVL | NM_016337 | 0.01286 | 0.20257 | 0.08039 | 0.22186 | 0.10932 | 0.22508 |
| UBE4A | NM_001204077 | 0.14791 | 0.11897 | 0.20900 | 0.07074 | 0.25402 | 0.05466 |
| LAD1 | NM_005558 | 0.08039 | 0.02894 | 0.07395 | 0.27653 | 0.08682 | 0.30868 |
| REP15 | NM_001029874 | 0.19936 | 0.16399 | 0.10611 | 0.12862 | 0.14148 | 0.11576 |
| GNGT2 | NM_001198754 | 0.03537 | 0.24759 | 0.08039 | 0.18006 | 0.07717 | 0.23473 |
| IRGC | NM_019612 | 0.38264 | 0.14148 | 0.19614 | 0.01608 | 0.10289 | 0.01608 |
| TACR1 | NM_001058 | 0.10289 | 0.22508 | 0.19293 | 0.15756 | 0.06431 | 0.11254 |
| CRISP3 | NM_001190986 | 0.03215 | 0.14148 | 0.00000 | 0.34084 | 0.01929 | 0.32154 |
| POTEH | NM_001136213 | 0.10289 | 0.29260 | 0.09968 | 0.14148 | 0.07717 | 0.14148 |
| ZNF668 | NM_024706 | 0.16720 | 0.19293 | 0.24759 | 0.01286 | 0.23151 | 0.00643 |
| TACR2 | NM_001057 | 0.03859 | 0.30547 | 0.07074 | 0.12862 | 0.18650 | 0.12862 |
| FAM222B | NM_001288633 | 0.15434 | 0.19614 | 0.10611 | 0.11576 | 0.08039 | 0.20579 |
| GSDMB | NM_001042471 | 0.28939 | 0.34405 | 0.06752 | 0.06431 | 0.04180 | 0.05145 |
| ROS1 | NM_002944 | 0.00322 | 0.19614 | 0.01286 | 0.29904 | 0.01929 | 0.32797 |
| MAMSTR | NM_001130915 | 0.08360 | 0.35691 | 0.07717 | 0.11254 | 0.09646 | 0.13505 |
| SHISA5 | NM_001272066 | 0.31833 | 0.09646 | 0.10611 | 0.07717 | 0.16077 | 0.10289 |
| TRAF3IP1 | NM_015650 | 0.07717 | 0.00322 | 0.22830 | 0.04502 | 0.37942 | 0.13183 |
| PAM16 | NM_016069 | 0.00000 | 0.16720 | 0.03859 | 0.24116 | 0.06109 | 0.35691 |
| ATG4B | NM_178326 | 0.02894 | 0.31511 | 0.03859 | 0.27974 | 0.05466 | 0.14791 |
| ACKR1 | NM_001122951 | 0.18971 | 0.06109 | 0.15756 | 0.14791 | 0.16077 | 0.14791 |
| THOC7 | NM_025075 | 0.17363 | 0.07717 | 0.20257 | 0.06431 | 0.25723 | 0.09003 |
| RAB2A | NM_002865 | 0.09325 | 0.24759 | 0.07074 | 0.23473 | 0.06109 | 0.16077 |
| HNRNPF | NM_001098204 | 0.02572 | 0.05788 | 0.09003 | 0.27974 | 0.06109 | 0.35370 |
| SPRR1B | NM_003125 | 0.36656 | 0.11576 | 0.19936 | 0.05145 | 0.06752 | 0.06752 |
| RUNDC3B | NM_138290 | 0.24437 | 0.28617 | 0.08360 | 0.10611 | 0.07717 | 0.07395 |
| SPTBN2 | NM_006946 | 0.09968 | 0.44373 | 0.04180 | 0.17685 | 0.05466 | 0.05466 |
| AGK | NM_018238 | 0.27974 | 0.16077 | 0.05788 | 0.15756 | 0.06752 | 0.14791 |
| PTPRR | NM_001207015 | 0.10932 | 0.08682 | 0.10611 | 0.21222 | 0.13826 | 0.21865 |
| UGT1A9 | NM_021027 | 0.00000 | 0.24759 | 0.04180 | 0.25723 | 0.09325 | 0.23473 |
| ALPPL2 | NM_031313 | 0.13826 | 0.04502 | 0.28617 | 0.07395 | 0.24116 | 0.09003 |
| FOXI1 | NM_012188 | 0.00000 | 0.37621 | 0.03859 | 0.15756 | 0.05145 | 0.25080 |
| UHRF1BP1L | NM_015054 | 0.03859 | 0.14148 | 0.01286 | 0.36977 | 0.03537 | 0.27653 |
| EMC4 | NM_001286420 | 0.28939 | 0.15756 | 0.15756 | 0.02572 | 0.22186 | 0.02251 |
| SLC7A13 | NM_138817 | 0.00000 | 0.22186 | 0.00643 | 0.28939 | 0.06431 | 0.29260 |
| HABP4 | NM_014282 | 0.43408 | 0.13183 | 0.19293 | 0.02572 | 0.07717 | 0.01608 |
| LHX3 | NM_178138 | 0.04502 | 0.18328 | 0.08039 | 0.24437 | 0.05466 | 0.27331 |
| NREP | NM_001142482 | 0.22830 | 0.10611 | 0.03537 | 0.20900 | 0.04180 | 0.26045 |
| TAAR5 | NM_003967 | 0.17363 | 0.25723 | 0.18971 | 0.07717 | 0.13183 | 0.05145 |
| FAM69A | NM_001252271 | 0.05466 | 0.24759 | 0.13505 | 0.18650 | 0.18650 | 0.07395 |
| KDM2A | NM_001256405 | 0.16399 | 0.36656 | 0.22830 | 0.04502 | 0.05466 | 0.02572 |
| GTF2B | NM_001514 | 0.12540 | 0.13826 | 0.16399 | 0.07395 | 0.28296 | 0.09968 |
| PCDHB15 | NM_018935 | 0.19936 | 0.13183 | 0.24759 | 0.00965 | 0.28296 | 0.01608 |
| DUT | NM_001025249 | 0.00000 | 0.22508 | 0.03859 | 0.28939 | 0.05466 | 0.27974 |
| LRRC30 | NM_001105581 | 0.30225 | 0.19293 | 0.20579 | 0.02251 | 0.14791 | 0.01608 |
| NREP | NM_001142483 | 0.29260 | 0.33441 | 0.00643 | 0.12862 | 0.00322 | 0.12219 |
| KIAA0586 | NM_001244189 | 0.34727 | 0.14148 | 0.07717 | 0.11897 | 0.05466 | 0.14791 |
| AES | NM_198969 | 0.17685 | 0.22508 | 0.10611 | 0.06431 | 0.27653 | 0.03859 |
| COL11A1 | NM_001190709 | 0.03215 | 0.33119 | 0.09646 | 0.13826 | 0.14148 | 0.14791 |
| LSMEM2 | NM_153215 | 0.13826 | 0.23151 | 0.05466 | 0.20257 | 0.05466 | 0.20900 |
| CD72 | NM_001782 | 0.15756 | 0.09646 | 0.07395 | 0.19614 | 0.22508 | 0.14148 |
| IFNA10 | NM_002171 | 0.12540 | 0.36334 | 0.08039 | 0.15756 | 0.04823 | 0.11897 |
| MFSD5 | NM_001170790 | 0.44051 | 0.07395 | 0.19293 | 0.00965 | 0.16720 | 0.01286 |
| RBMXL2 | NM_014469 | 0.10289 | 0.07717 | 0.27653 | 0.00965 | 0.42122 | 0.00965 |
| OR5M3 | NM_001004742 | 0.03859 | 0.36334 | 0.03859 | 0.21543 | 0.05466 | 0.18650 |
| SCNN1G | NM_001039 | 0.11576 | 0.11897 | 0.10611 | 0.25402 | 0.08682 | 0.21543 |
| VHLL | NM_001004319 | 0.34405 | 0.13183 | 0.06431 | 0.13826 | 0.05788 | 0.16077 |
| VSIG4 | NM_001100431 | 0.15434 | 0.44373 | 0.10611 | 0.07395 | 0.06431 | 0.05466 |
| SOWAHC | NM_023016 | 0.02251 | 0.13183 | 0.18006 | 0.22186 | 0.18650 | 0.15434 |
| LMX1B | NM_001174147 | 0.27653 | 0.00643 | 0.35048 | 0.00965 | 0.24437 | 0.01286 |
| EBP | NM_006579 | 0.05466 | 0.13183 | 0.00643 | 0.33441 | 0.01929 | 0.35370 |
| NALCN | NM_052867 | 0.06109 | 0.46624 | 0.00965 | 0.13826 | 0.01608 | 0.20900 |
| WNK4 | NM_032387 | 0.05466 | 0.35370 | 0.09646 | 0.13826 | 0.14791 | 0.10932 |
| PANX1 | NM_015368 | 0.04180 | 0.30547 | 0.03859 | 0.26688 | 0.06109 | 0.18650 |
| AGPAT1 | NM_032741 | 0.16399 | 0.14148 | 0.11576 | 0.10932 | 0.19293 | 0.17685 |
| RPL26 | NM_000987 | 0.04502 | 0.26688 | 0.04180 | 0.22508 | 0.07074 | 0.25402 |
| PPP1R27 | NM_001007533 | 0.28617 | 0.14148 | 0.15756 | 0.06752 | 0.07074 | 0.18006 |
| GPBP1 | NM_001127236 | 0.38585 | 0.04502 | 0.21543 | 0.02894 | 0.17363 | 0.05466 |
| PLEC | NM_201378 | 0.09003 | 0.08682 | 0.23473 | 0.10611 | 0.22186 | 0.16720 |
| LARS | NM_020117 | 0.30547 | 0.00322 | 0.19614 | 0.00965 | 0.37942 | 0.01286 |
| HSF4 | NM_001538 | 0.15756 | 0.07717 | 0.17685 | 0.10611 | 0.23794 | 0.15113 |
| ARFGAP2 | NM_032389 | 0.12219 | 0.08039 | 0.15434 | 0.17685 | 0.16077 | 0.21222 |
| FAM96B | NM_016062 | 0.14469 | 0.00643 | 0.29582 | 0.06431 | 0.25723 | 0.13826 |
| NKX2-2 | NM_002509 | 0.21865 | 0.09003 | 0.08039 | 0.19293 | 0.09325 | 0.23473 |
| MEFV | NM_001198536 | 0.43730 | 0.17042 | 0.17685 | 0.04502 | 0.03537 | 0.04502 |
| TMEM56 | NM_001199679 | 0.26045 | 0.00322 | 0.28617 | 0.06431 | 0.24116 | 0.05466 |
| FZR1 | NM_016263 | 0.08360 | 0.02251 | 0.21543 | 0.03859 | 0.38264 | 0.16720 |
| TMEM170A | NM_145254 | 0.32797 | 0.05466 | 0.17363 | 0.13826 | 0.09325 | 0.12219 |
| OR2B2 | NM_033057 | 0.44051 | 0.00322 | 0.28617 | 0.00965 | 0.16077 | 0.01286 |
| ZNF331 | NM_001079906 | 0.01286 | 0.13183 | 0.08039 | 0.27974 | 0.15756 | 0.25080 |
| PGBD2 | NM_170725 | 0.04823 | 0.30547 | 0.03859 | 0.24437 | 0.03859 | 0.23794 |
| ARFIP2 | NM_012402 | 0.29582 | 0.04502 | 0.19936 | 0.03537 | 0.28296 | 0.05466 |
| EBF1 | NM_024007 | 0.18971 | 0.24759 | 0.08039 | 0.12862 | 0.05466 | 0.21222 |
| PRM1 | NM_002761 | 0.00000 | 0.16077 | 0.10611 | 0.17363 | 0.35691 | 0.11576 |
| ARHGAP26 | NM_015071 | 0.22830 | 0.13183 | 0.12219 | 0.14148 | 0.15113 | 0.13826 |
| AASDH | NM_181806 | 0.15113 | 0.14148 | 0.19614 | 0.07395 | 0.29904 | 0.05145 |
| CLN3 | NM_001286104 | 0.28296 | 0.14148 | 0.17685 | 0.09646 | 0.14791 | 0.06752 |
| PEX26 | NM_001127649 | 0.00965 | 0.36334 | 0.07074 | 0.20257 | 0.14469 | 0.12540 |
| ECSCR | NM_001077693 | 0.02572 | 0.13183 | 0.17363 | 0.04502 | 0.49518 | 0.04502 |
| ASGR2 | NM_080913 | 0.38264 | 0.14148 | 0.19614 | 0.02572 | 0.12219 | 0.04823 |
| MPPED2 | NM_001584 | 0.11254 | 0.16077 | 0.19293 | 0.09646 | 0.21865 | 0.13505 |
| FBXO7 | NM_012179 | 0.05788 | 0.36977 | 0.03859 | 0.20257 | 0.03537 | 0.21222 |
| CNGB3 | NM_019098 | 0.29582 | 0.14148 | 0.08039 | 0.17685 | 0.04823 | 0.17363 |
| GKAP1 | NM_001135953 | 0.27653 | 0.09003 | 0.22830 | 0.04502 | 0.25723 | 0.02251 |
| ITIH1 | NM_001166434 | 0.15434 | 0.26367 | 0.08682 | 0.18006 | 0.09325 | 0.14148 |
| C1QTNF3 | NM_181435 | 0.01286 | 0.39871 | 0.02572 | 0.25402 | 0.05466 | 0.17363 |
| FAM170A | NM_182761 | 0.44051 | 0.06109 | 0.22830 | 0.05788 | 0.08682 | 0.04502 |
| PHYHIP | NM_001099335 | 0.14791 | 0.09646 | 0.29582 | 0.07395 | 0.20900 | 0.09646 |
| PCTP | NM_021213 | 0.10289 | 0.14148 | 0.09325 | 0.23151 | 0.13826 | 0.21222 |
| OR5P2 | NM_153444 | 0.09003 | 0.04502 | 0.32154 | 0.05466 | 0.35370 | 0.05788 |
| ASIC3 | NM_004769 | 0.08360 | 0.20257 | 0.27010 | 0.13183 | 0.17685 | 0.05788 |
| SYNE3 | NM_152592 | 0.51447 | 0.17685 | 0.12862 | 0.02572 | 0.06109 | 0.01608 |
| NAT16 | NM_198571 | 0.38264 | 0.07717 | 0.19293 | 0.05788 | 0.16077 | 0.05145 |
| YOD1 | NM_001276320 | 0.01929 | 0.34727 | 0.02572 | 0.20257 | 0.03537 | 0.29260 |
| MANEA | NM_024641 | 0.00000 | 0.37299 | 0.00000 | 0.25723 | 0.00000 | 0.29260 |
| LTV1 | NM_032860 | 0.34405 | 0.04502 | 0.26688 | 0.04502 | 0.18650 | 0.03859 |
| ADAMTSL1 | NM_001040272 | 0.02572 | 0.06431 | 0.00643 | 0.33762 | 0.01608 | 0.47588 |
| AMER2 | NM_152704 | 0.09003 | 0.34405 | 0.10611 | 0.08682 | 0.11576 | 0.18650 |
| OR2W5 | NM_001004698 | 0.24437 | 0.03215 | 0.22830 | 0.04502 | 0.34084 | 0.03859 |
| PARP15 | NM_001113523 | 0.09003 | 0.17042 | 0.03859 | 0.29582 | 0.04180 | 0.29260 |
| PIK3C2G | NM_001288772 | 0.10289 | 0.16399 | 0.08360 | 0.31190 | 0.05466 | 0.21222 |
| LY6E | NM_002346 | 0.47910 | 0.05145 | 0.29904 | 0.02572 | 0.05788 | 0.01608 |
| PPT1 | NM_000310 | 0.44373 | 0.03215 | 0.15756 | 0.03537 | 0.20257 | 0.05788 |
| UPP1 | NM_003364 | 0.12862 | 0.33119 | 0.17363 | 0.06431 | 0.19614 | 0.03537 |
| ZBTB49 | NM_145291 | 0.25080 | 0.22508 | 0.08039 | 0.17685 | 0.05466 | 0.14148 |
| LOXL1 | NM_005576 | 0.04823 | 0.31190 | 0.15756 | 0.11897 | 0.20900 | 0.08682 |
| C17orf49 | NM_001142798 | 0.39228 | 0.00322 | 0.27331 | 0.02572 | 0.18971 | 0.04823 |
| DDX47 | NM_016355 | 0.45659 | 0.02894 | 0.19936 | 0.00965 | 0.22508 | 0.01286 |
| SERINC1 | NM_020755 | 0.05788 | 0.22508 | 0.03859 | 0.31511 | 0.05788 | 0.23794 |
| SP5 | NM_001003845 | 0.03859 | 0.18006 | 0.03859 | 0.28939 | 0.05466 | 0.33119 |
| FOXP3 | NM_014009 | 0.12219 | 0.07717 | 0.17363 | 0.13505 | 0.17363 | 0.25080 |
| PHF19 | NM_001286843 | 0.08682 | 0.14148 | 0.12219 | 0.21543 | 0.20900 | 0.16077 |
| SYNGR4 | NM_012451 | 0.08682 | 0.46302 | 0.03859 | 0.18006 | 0.01286 | 0.15434 |
| BTBD3 | NM_014962 | 0.09968 | 0.23151 | 0.20579 | 0.08682 | 0.21865 | 0.09325 |
| AATK | NM_001080395 | 0.04823 | 0.35691 | 0.10611 | 0.15756 | 0.11897 | 0.14791 |
| GPR128 | NM_032787 | 0.23151 | 0.11576 | 0.08039 | 0.16399 | 0.15113 | 0.19293 |
| ETFDH | NM_001281738 | 0.02894 | 0.13826 | 0.04823 | 0.25723 | 0.06752 | 0.39550 |
| ASB15 | NM_080928 | 0.18650 | 0.20257 | 0.17363 | 0.07717 | 0.18650 | 0.10932 |
| FOXP1 | NM_001244808 | 0.15756 | 0.34727 | 0.08039 | 0.13826 | 0.06431 | 0.14791 |
| CCDC34 | NM_030771 | 0.04823 | 0.08682 | 0.28939 | 0.06431 | 0.33441 | 0.11576 |
| CACFD1 | NM_017586 | 0.12540 | 0.22186 | 0.03859 | 0.29260 | 0.04823 | 0.21222 |
| RMDN1 | NM_001286719 | 0.06431 | 0.21543 | 0.19293 | 0.07717 | 0.28296 | 0.10932 |
| OPRL1 | NM_000913 | 0.02251 | 0.14469 | 0.02572 | 0.28296 | 0.06109 | 0.40514 |
| ZNF540 | NM_152606 | 0.03859 | 0.36334 | 0.00000 | 0.22186 | 0.00000 | 0.32154 |
| IQCD | NM_138451 | 0.05145 | 0.56913 | 0.03859 | 0.13505 | 0.06752 | 0.08360 |
| FAM9B | NM_205849 | 0.11576 | 0.34405 | 0.08360 | 0.21222 | 0.08682 | 0.10289 |
| ARHGAP25 | NM_001166277 | 0.32797 | 0.16077 | 0.12540 | 0.14469 | 0.05788 | 0.12862 |
| STC1 | NM_003155 | 0.12219 | 0.11576 | 0.20579 | 0.02894 | 0.42765 | 0.04823 |
| PPP2R2C | NM_181876 | 0.34084 | 0.03859 | 0.22830 | 0.04502 | 0.24437 | 0.05145 |
| CDRT1 | NM_001282540 | 0.03859 | 0.39871 | 0.14791 | 0.11254 | 0.18971 | 0.06109 |
| STK32B | NM_018401 | 0.08682 | 0.19614 | 0.18328 | 0.13826 | 0.24437 | 0.09968 |
| P2RY10 | NM_014499 | 0.08360 | 0.11897 | 0.14791 | 0.24437 | 0.13183 | 0.22508 |
| P2RX5 | NM_001204519 | 0.01929 | 0.68489 | 0.05466 | 0.07395 | 0.10289 | 0.01608 |
| B4GALT2 | NM_003780 | 0.32797 | 0.08682 | 0.08039 | 0.17685 | 0.07717 | 0.20257 |
| C16orf59 | NM_025108 | 0.03859 | 0.42122 | 0.06752 | 0.18006 | 0.04823 | 0.19614 |
| FOXD4 | NM_207305 | 0.02251 | 0.32154 | 0.08039 | 0.21543 | 0.15113 | 0.16077 |
| RPL10L | NM_080746 | 0.06109 | 0.09646 | 0.05466 | 0.33762 | 0.02572 | 0.37621 |
| PLS1 | NM_001145319 | 0.09325 | 0.32797 | 0.03859 | 0.27974 | 0.05466 | 0.16077 |
| GALE | NM_001127621 | 0.07717 | 0.37942 | 0.03859 | 0.17685 | 0.04180 | 0.24116 |
| RAB9B | NM_016370 | 0.52090 | 0.00322 | 0.30225 | 0.01286 | 0.10289 | 0.01286 |
| CREG2 | NM_153836 | 0.37942 | 0.05788 | 0.22508 | 0.00322 | 0.28296 | 0.00643 |
| MTFP1 | NM_016498 | 0.09003 | 0.17042 | 0.19614 | 0.07717 | 0.30225 | 0.12219 |
| GP1BB | NM_000407 | 0.02251 | 0.35691 | 0.01286 | 0.43730 | 0.04823 | 0.08039 |
| FLYWCH2 | NM_001142499 | 0.17363 | 0.13183 | 0.08039 | 0.26367 | 0.04823 | 0.26045 |
| MPI | NM_002435 | 0.09003 | 0.16077 | 0.12219 | 0.21865 | 0.11576 | 0.25080 |
| ZFP36L2 | NM_006887 | 0.42444 | 0.03215 | 0.12540 | 0.13505 | 0.09325 | 0.14791 |
| KCNJ15 | NM_170736 | 0.10932 | 0.19293 | 0.08039 | 0.19936 | 0.15113 | 0.22508 |
| KIF17 | NM_001122819 | 0.02894 | 0.29582 | 0.04180 | 0.23473 | 0.06431 | 0.29260 |
| SNRPB2 | NM_003092 | 0.02894 | 0.32154 | 0.06431 | 0.29582 | 0.09968 | 0.14791 |
| THBD | NM_000361 | 0.12219 | 0.17042 | 0.15113 | 0.13826 | 0.18650 | 0.19293 |
| TOMM34 | NM_006809 | 0.00965 | 0.26045 | 0.03859 | 0.42444 | 0.06431 | 0.16399 |
| PPARG | NM_015869 | 0.11576 | 0.04502 | 0.15756 | 0.17363 | 0.30225 | 0.16720 |
| GNG7 | NM_052847 | 0.21865 | 0.45659 | 0.07395 | 0.07395 | 0.04823 | 0.09003 |
| TMEM125 | NM_144626 | 0.02251 | 0.30225 | 0.03859 | 0.28296 | 0.05466 | 0.26045 |
| PSTK | NM_153336 | 0.30225 | 0.13826 | 0.15756 | 0.05788 | 0.19614 | 0.10932 |
| AMPD2 | NM_001257361 | 0.23151 | 0.13183 | 0.18006 | 0.13183 | 0.08039 | 0.20900 |
| ZNF648 | NM_001009992 | 0.04823 | 0.29260 | 0.07395 | 0.25723 | 0.05466 | 0.23794 |
| ETFA | NM_001127716 | 0.29582 | 0.27010 | 0.12219 | 0.05145 | 0.16077 | 0.06431 |
| ZMYM3 | NM_001171162 | 0.41801 | 0.29582 | 0.09646 | 0.04502 | 0.05466 | 0.05466 |
| ZNF827 | NM_178835 | 0.06752 | 0.19293 | 0.35048 | 0.03215 | 0.28296 | 0.03859 |
| SPAG8 | NM_172312 | 0.00000 | 0.32797 | 0.00000 | 0.28939 | 0.01608 | 0.33119 |
| PDIA6 | NM_001282705 | 0.37942 | 0.24759 | 0.12540 | 0.07717 | 0.05466 | 0.08360 |
| NTNG1 | NM_001113228 | 0.34727 | 0.08039 | 0.08360 | 0.19614 | 0.04823 | 0.21222 |
| SLC1A5 | NM_001145145 | 0.10289 | 0.19293 | 0.09968 | 0.17042 | 0.24437 | 0.15756 |
| ZC3H15 | NM_018471 | 0.02894 | 0.01286 | 0.12540 | 0.26688 | 0.03537 | 0.50161 |
| MRPL22 | NM_014180 | 0.11897 | 0.03859 | 0.15434 | 0.27331 | 0.10932 | 0.27653 |
| ORC6 | NM_014321 | 0.04180 | 0.30547 | 0.03859 | 0.26688 | 0.05466 | 0.26367 |
| HSPB7 | NM_014424 | 0.09325 | 0.34405 | 0.17363 | 0.05145 | 0.29582 | 0.01286 |
| IKZF4 | NM_022465 | 0.03537 | 0.45659 | 0.17042 | 0.05466 | 0.22508 | 0.02894 |
| FAM49A | NM_030797 | 0.07717 | 0.23473 | 0.15434 | 0.03537 | 0.43087 | 0.03859 |
| TBC1D16 | NM_019020 | 0.09003 | 0.33441 | 0.19293 | 0.15756 | 0.09646 | 0.09968 |
| C12orf77 | NM_001101339 | 0.11576 | 0.02251 | 0.20900 | 0.15113 | 0.31511 | 0.16077 |
| RAPGEF1 | NM_198679 | 0.65273 | 0.09646 | 0.12540 | 0.03215 | 0.05145 | 0.01608 |
| STEAP1B | NM_207342 | 0.37621 | 0.16720 | 0.18971 | 0.00965 | 0.22186 | 0.00965 |
| POLR3H | NM_001018052 | 0.25402 | 0.05466 | 0.18006 | 0.11897 | 0.22186 | 0.14469 |
| SHOC2 | NM_007373 | 0.10289 | 0.16077 | 0.28939 | 0.03215 | 0.30225 | 0.08682 |
| ECHDC2 | NM_018281 | 0.10289 | 0.14148 | 0.14791 | 0.24437 | 0.07074 | 0.26688 |
| KRTAP4-7 | NM_033061 | 0.52090 | 0.14469 | 0.19936 | 0.02894 | 0.05466 | 0.02572 |
| HELZ2 | NM_033405 | 0.02251 | 0.30225 | 0.08039 | 0.18006 | 0.13826 | 0.25080 |
| PDK2 | NM_001199900 | 0.00965 | 0.04502 | 0.22830 | 0.07074 | 0.55949 | 0.06431 |
| TBC1D21 | NM_153356 | 0.29582 | 0.23151 | 0.13826 | 0.07717 | 0.16077 | 0.07395 |
| GFPT2 | NM_005110 | 0.02894 | 0.09325 | 0.17363 | 0.17685 | 0.25080 | 0.25402 |
| MEX3A | NM_001093725 | 0.28617 | 0.36013 | 0.03859 | 0.06752 | 0.14148 | 0.08682 |
| PPP1R10 | NM_002714 | 0.05466 | 0.33119 | 0.08039 | 0.23473 | 0.10932 | 0.17042 |
| GPATCH3 | NM_022078 | 0.38264 | 0.13183 | 0.19614 | 0.02894 | 0.20257 | 0.03859 |
| SMIM14 | NM_174921 | 0.00965 | 0.06752 | 0.03859 | 0.32476 | 0.06431 | 0.47588 |
| MEF2A | NM_005587 | 0.33762 | 0.22186 | 0.11897 | 0.03215 | 0.22186 | 0.04823 |
| ZNF609 | NM_015042 | 0.08360 | 0.17363 | 0.27010 | 0.07395 | 0.27331 | 0.10611 |
| KIAA1377 | NM_020802 | 0.37942 | 0.02894 | 0.27653 | 0.03215 | 0.24116 | 0.02251 |
| RLTPR | NM_001013838 | 0.02251 | 0.35691 | 0.04180 | 0.18650 | 0.06431 | 0.30868 |
| GGA3 | NM_001291642 | 0.27974 | 0.20257 | 0.12540 | 0.10611 | 0.16077 | 0.10932 |
| ANK1 | NM_001142446 | 0.44373 | 0.05145 | 0.31190 | 0.03215 | 0.12862 | 0.01608 |
| SLC15A4 | NM_145648 | 0.37942 | 0.07717 | 0.22186 | 0.00965 | 0.28296 | 0.01286 |
| TMEM52 | NM_178545 | 0.31833 | 0.22186 | 0.17042 | 0.05145 | 0.14791 | 0.07395 |
| RTN2 | NM_206900 | 0.42765 | 0.09003 | 0.19936 | 0.02894 | 0.22508 | 0.01286 |
| RASGRP1 | NM_001128602 | 0.06752 | 0.17042 | 0.11254 | 0.25723 | 0.14791 | 0.22830 |
| YWHAZ | NM_001135699 | 0.00965 | 0.33441 | 0.00000 | 0.35370 | 0.03537 | 0.25080 |
| PRRX1 | NM_006902 | 0.06431 | 0.11576 | 0.03859 | 0.40836 | 0.05466 | 0.30225 |
| EFCAB2 | NM_032328 | 0.37299 | 0.13183 | 0.03859 | 0.18650 | 0.06109 | 0.19293 |
| ZNF185 | NM_001178113 | 0.44695 | 0.07717 | 0.35048 | 0.02572 | 0.07395 | 0.00965 |
| SP100 | NM_003113 | 0.37621 | 0.36334 | 0.07395 | 0.06752 | 0.01608 | 0.09003 |
| NCBP1 | NM_002486 | 0.58199 | 0.11576 | 0.19293 | 0.01608 | 0.05466 | 0.02572 |
| ASNSD1 | NM_019048 | 0.31190 | 0.18328 | 0.27010 | 0.00965 | 0.20257 | 0.00965 |
| SFT2D3 | NM_032740 | 0.38264 | 0.07717 | 0.20900 | 0.02894 | 0.23794 | 0.05145 |
| CYBB | NM_000397 | 0.15756 | 0.22508 | 0.17042 | 0.18650 | 0.06109 | 0.18650 |
| ST20- | NM_001199760 | 0.29260 | 0.07717 | 0.29904 | 0.00965 | 0.29260 | 0.01608 |
| MTHFS | | | | | | | |
| GABARAP | NM_007278 | 0.10289 | 0.00322 | 0.36977 | 0.04823 | 0.41158 | 0.05145 |
| KIF25 | NM_030615 | 0.41158 | 0.22186 | 0.18971 | 0.07395 | 0.05466 | 0.03537 |
| CNTNAP4 | NM_033401 | 0.29260 | 0.02894 | 0.28939 | 0.00965 | 0.35370 | 0.01286 |
| EPHA3 | NM_182644 | 0.00000 | 0.20579 | 0.09325 | 0.27974 | 0.05466 | 0.35691 |
| FIBP | NM_004214 | 0.15756 | 0.22186 | 0.19293 | 0.11897 | 0.22508 | 0.07395 |
| SLC7A11 | NM_014331 | 0.10289 | 0.00322 | 0.36977 | 0.02894 | 0.43730 | 0.04823 |
| MRPS17 | NM_015969 | 0.34084 | 0.15113 | 0.14148 | 0.09646 | 0.13826 | 0.12219 |
| FAM83B | NM_001010872 | 0.09003 | 0.30547 | 0.04502 | 0.25402 | 0.04502 | 0.25402 |
| BCOR | NM_001123384 | 0.34405 | 0.06752 | 0.19936 | 0.13826 | 0.12219 | 0.12219 |
| NKX3-2 | NM_001189 | 0.42765 | 0.16720 | 0.08039 | 0.11254 | 0.06431 | 0.14148 |
| CLTCL1 | NM_001835 | 0.10289 | 0.08039 | 0.17042 | 0.21543 | 0.10289 | 0.32154 |
| HOXB13 | NM_006361 | 0.37942 | 0.14148 | 0.15756 | 0.07074 | 0.18650 | 0.05788 |
| SLITRK6 | NM_032229 | 0.01286 | 0.31190 | 0.03859 | 0.18006 | 0.05466 | 0.39871 |
| VNN1 | NM_004666 | 0.44373 | 0.05145 | 0.19293 | 0.07395 | 0.08682 | 0.14791 |
| C9orf117 | NM_001012502 | 0.04823 | 0.22186 | 0.24116 | 0.07717 | 0.33441 | 0.07395 |
| RHPN1 | NM_052924 | 0.03859 | 0.11576 | 0.03859 | 0.29582 | 0.10289 | 0.40514 |
| VWA5A | NM_014622 | 0.34405 | 0.00643 | 0.29582 | 0.03537 | 0.28939 | 0.02572 |
| VEGFA | NM_001171630 | 0.09003 | 0.16399 | 0.12540 | 0.32476 | 0.09968 | 0.19614 |
| TMEM225 | NM_001013743 | 0.10289 | 0.13183 | 0.12219 | 0.25402 | 0.14469 | 0.24437 |
| INPP4B | NM_003866 | 0.37942 | 0.16077 | 0.16399 | 0.04502 | 0.18650 | 0.06431 |
| EID1 | NM_014335 | 0.08360 | 0.11576 | 0.17685 | 0.11897 | 0.34727 | 0.15756 |
| CDH19 | NM_021153 | 0.05788 | 0.24759 | 0.10932 | 0.22186 | 0.12219 | 0.24116 |
| COX6B2 | NM_144613 | 0.12540 | 0.01286 | 0.34084 | 0.05788 | 0.42122 | 0.04180 |
| SS18L1 | NM_198935 | 0.29260 | 0.13183 | 0.13183 | 0.11897 | 0.13826 | 0.18650 |
| CDKN2B | NM_004936 | 0.14791 | 0.24759 | 0.15113 | 0.13826 | 0.22186 | 0.09325 |
| ECHDC3 | NM_024693 | 0.36656 | 0.08682 | 0.23151 | 0.06431 | 0.18971 | 0.06109 |
| RIOK2 | NM_018343 | 0.38907 | 0.15434 | 0.10611 | 0.06431 | 0.14791 | 0.13826 |
| IGFBP4 | NM_001552 | 0.27653 | 0.07395 | 0.08682 | 0.18971 | 0.09325 | 0.28296 |
| TAF7L | NM_024885 | 0.24437 | 0.17042 | 0.18971 | 0.11897 | 0.23151 | 0.04823 |
| KRT83 | NM_002282 | 0.27653 | 0.37299 | 0.08039 | 0.09325 | 0.07074 | 0.10932 |
| HRH1 | NM_001098212 | 0.44695 | 0.07717 | 0.13183 | 0.12862 | 0.06109 | 0.15756 |
| PIN4 | NM_001170747 | 0.41801 | 0.01286 | 0.19614 | 0.03537 | 0.28939 | 0.05145 |
| CHRD | NM_003741 | 0.14791 | 0.19293 | 0.20900 | 0.15434 | 0.18650 | 0.11254 |
| PABPN1 | NM_004643 | 0.18971 | 0.07717 | 0.17685 | 0.17685 | 0.21865 | 0.16399 |
| TSSK3 | NM_052841 | 0.04180 | 0.00322 | 0.45981 | 0.01608 | 0.43087 | 0.05466 |
| OAF | NM_178507 | 0.04823 | 0.34405 | 0.09325 | 0.27974 | 0.05466 | 0.18650 |
| SPINK8 | NM_001080525 | 0.28617 | 0.09325 | 0.19614 | 0.15756 | 0.09646 | 0.17685 |
| CAPN1 | NM_005186 | 0.12862 | 0.05145 | 0.29582 | 0.17685 | 0.24437 | 0.10932 |
| HIGD1B | NM_016438 | 0.02572 | 0.00643 | 0.29582 | 0.00965 | 0.65916 | 0.00965 |
| TM7SF3 | NM_016551 | 0.37621 | 0.20257 | 0.17042 | 0.02894 | 0.19293 | 0.03537 |
| SYNCRIP | NM_006372 | 0.03859 | 0.16077 | 0.13505 | 0.23473 | 0.14791 | 0.28939 |
| OR4S2 | NM_001004059 | 0.03859 | 0.13183 | 0.01286 | 0.38907 | 0.01608 | 0.42122 |
| SLC2A8 | NM_014580 | 0.28617 | 0.32154 | 0.22186 | 0.00965 | 0.16077 | 0.00965 |
| MAGEB17 | NM_001277307 | 0.13183 | 0.13183 | 0.09646 | 0.20579 | 0.28296 | 0.16077 |
| PLA2G7 | NM_001168357 | 0.37299 | 0.24759 | 0.08039 | 0.06752 | 0.04823 | 0.19293 |
| PLK4 | NM_001190799 | 0.02572 | 0.13505 | 0.03859 | 0.35048 | 0.09646 | 0.36334 |
| HAUS6 | NM_001270890 | 0.12540 | 0.21543 | 0.17685 | 0.11897 | 0.23794 | 0.13505 |
| PEX3 | NM_003630 | 0.15756 | 0.42122 | 0.15756 | 0.13505 | 0.06109 | 0.07717 |
| PDXDC1 | NM_001285448 | 0.00965 | 0.30547 | 0.04823 | 0.17042 | 0.16077 | 0.31833 |
| NIPA1 | NM_001142275 | 0.07074 | 0.41801 | 0.03859 | 0.22186 | 0.05466 | 0.20900 |
| NFATC3 | NM_173165 | 0.07074 | 0.30547 | 0.09325 | 0.24437 | 0.04823 | 0.25080 |
| CAPN3 | NM_173088 | 0.19936 | 0.06431 | 0.07395 | 0.25402 | 0.06109 | 0.36013 |
| ZNF385B | NM_001282725 | 0.03859 | 0.22508 | 0.19936 | 0.05466 | 0.43408 | 0.06431 |
| ANAPC1 | NM_022662 | 0.29260 | 0.13183 | 0.19614 | 0.11254 | 0.16077 | 0.12219 |
| AKAP13 | NM_001270546 | 0.29260 | 0.09646 | 0.04180 | 0.26367 | 0.03537 | 0.28617 |
| C17orf67 | NM_001085430 | 0.05145 | 0.16399 | 0.13183 | 0.25402 | 0.19293 | 0.22508 |
| PHTF2 | NM_001127358 | 0.04502 | 0.12219 | 0.15756 | 0.17685 | 0.24116 | 0.27653 |
| SETDB2 | NM_001160308 | 0.10289 | 0.33441 | 0.00322 | 0.38907 | 0.00322 | 0.18650 |
| NBL1 | NM_001278166 | 0.07717 | 0.30547 | 0.21543 | 0.06752 | 0.31833 | 0.03537 |
| C6orf15 | NM_014070 | 0.00000 | 0.53698 | 0.04180 | 0.22186 | 0.09646 | 0.12219 |
| PLCB2 | NM_001284297 | 0.02572 | 0.33441 | 0.18006 | 0.16720 | 0.17363 | 0.13826 |
| CDK5RAP1 | NM_016082 | 0.41801 | 0.13826 | 0.22186 | 0.03537 | 0.13826 | 0.06752 |
| FBXO6 | NM_018438 | 0.29260 | 0.30547 | 0.17042 | 0.05145 | 0.14791 | 0.05466 |
| HIST1H1D | NM_005320 | 0.46624 | 0.16077 | 0.18650 | 0.02894 | 0.12219 | 0.05788 |
| FAM124B | NM_024785 | 0.43730 | 0.07395 | 0.18971 | 0.06752 | 0.14148 | 0.11254 |
| KRR1 | NM_007043 | 0.15434 | 0.28617 | 0.01286 | 0.28939 | 0.01929 | 0.26045 |
| ZNF93 | NM_031218 | 0.22186 | 0.13826 | 0.22186 | 0.08682 | 0.22508 | 0.12862 |
| ABHD13 | NM_032859 | 0.08360 | 0.30547 | 0.13505 | 0.16399 | 0.18650 | 0.14791 |
| DZIP1L | NM_173543 | 0.69132 | 0.03859 | 0.14791 | 0.03537 | 0.06109 | 0.04823 |
| HCRTR2 | NM_001526 | 0.10611 | 0.00322 | 0.29582 | 0.05788 | 0.48553 | 0.07395 |
| CUTC | NM_015960 | 0.12540 | 0.22508 | 0.09325 | 0.25402 | 0.09325 | 0.23473 |
| TRIM48 | NM_024114 | 0.54984 | 0.07717 | 0.19614 | 0.02894 | 0.15756 | 0.01608 |
| FBXO36 | NM_174899 | 0.44051 | 0.16399 | 0.22830 | 0.06431 | 0.09003 | 0.03859 |
| PIWIL2 | NM_001135721 | 0.37621 | 0.19614 | 0.03859 | 0.17363 | 0.00000 | 0.24116 |
| AKR1C1 | NM_001353 | 0.06431 | 0.06431 | 0.15756 | 0.12862 | 0.39550 | 0.21543 |
| TCP10L | NM_144659 | 0.22186 | 0.22508 | 0.28939 | 0.03215 | 0.24437 | 0.01286 |
| HNRNPL | NM_001533 | 0.13505 | 0.36977 | 0.17685 | 0.07395 | 0.11576 | 0.15434 |
| C2orf42 | NM_017880 | 0.34084 | 0.13826 | 0.15113 | 0.13826 | 0.09968 | 0.15756 |
| TMEM14B | NM_001286489 | 0.43730 | 0.16399 | 0.19293 | 0.03215 | 0.18971 | 0.01286 |
| TET2 | NM_017628 | 0.07074 | 0.32154 | 0.12540 | 0.20257 | 0.12219 | 0.18650 |
| ZNF233 | NM_001207005 | 0.16399 | 0.02251 | 0.08039 | 0.31190 | 0.07074 | 0.37942 |
| ARAP2 | NM_015230 | 0.12219 | 0.14469 | 0.07395 | 0.26688 | 0.06752 | 0.35370 |
| CCDC122 | NM_144974 | 0.19614 | 0.14148 | 0.20900 | 0.07395 | 0.29904 | 0.10932 |
| REC8 | NM_001048205 | 0.11576 | 0.32476 | 0.00322 | 0.18006 | 0.01286 | 0.39550 |
| TULP2 | NM_003323 | 0.01929 | 0.23151 | 0.00322 | 0.35370 | 0.00000 | 0.42444 |
| CD83 | NM_004233 | 0.00965 | 0.49196 | 0.03859 | 0.20257 | 0.09646 | 0.19293 |
| TAF3 | NM_031923 | 0.12862 | 0.11897 | 0.19936 | 0.15756 | 0.28296 | 0.14791 |
| TGFB1I1 | NM_001042454 | 0.04502 | 0.07717 | 0.12219 | 0.11254 | 0.49839 | 0.18006 |
| DMXL1 | NM_005509 | 0.25080 | 0.16077 | 0.19293 | 0.06431 | 0.24437 | 0.12219 |
| ART5 | NM_001079536 | 0.04823 | 0.41801 | 0.10611 | 0.18650 | 0.07395 | 0.20257 |
| URI1 | NM_001252641 | 0.09003 | 0.30547 | 0.13505 | 0.14791 | 0.19614 | 0.16077 |
| AHCYL2 | NM_001130722 | 0.37621 | 0.00643 | 0.29582 | 0.05788 | 0.22508 | 0.07395 |
| HIST1H3A | NM_003529 | 0.01929 | 0.03215 | 0.00322 | 0.35370 | 0.06431 | 0.56270 |
| KREMEN1 | NM_032045 | 0.39871 | 0.10289 | 0.22186 | 0.03537 | 0.25402 | 0.02251 |
| RB1CC1 | NM_014781 | 0.07074 | 0.05466 | 0.23473 | 0.26688 | 0.16077 | 0.25080 |
| UBA5 | NM_024818 | 0.00000 | 0.24759 | 0.00643 | 0.26045 | 0.03537 | 0.48875 |
| FAM65C | NM_080829 | 0.01286 | 0.35691 | 0.10611 | 0.19936 | 0.15113 | 0.21222 |
| ATP5A1 | NM_001001937 | 0.28617 | 0.16077 | 0.18328 | 0.12862 | 0.20900 | 0.07395 |
| UHRF1 | NM_001290050 | 0.57235 | 0.01286 | 0.12219 | 0.07395 | 0.13183 | 0.12862 |
| CPNE4 | NM_153429 | 0.39228 | 0.03859 | 0.31833 | 0.03537 | 0.24116 | 0.01608 |
| RASSF3 | NM_178169 | 0.05466 | 0.20257 | 0.27010 | 0.01608 | 0.47267 | 0.02572 |
| PARP12 | NM_022750 | 0.07074 | 0.57556 | 0.03859 | 0.15756 | 0.05145 | 0.14791 |
| CCT4 | NM_001256721 | 0.17685 | 0.19293 | 0.16399 | 0.04502 | 0.28296 | 0.18006 |
| LMNA | NM_001257374 | 0.00000 | 0.14148 | 0.00000 | 0.42122 | 0.00000 | 0.47910 |
| RPN1 | NM_002950 | 0.17363 | 0.22508 | 0.09325 | 0.15756 | 0.14791 | 0.24437 |
| ZNF718 | NM_001039127 | 0.23151 | 0.09003 | 0.12862 | 0.18006 | 0.21865 | 0.19293 |
| PLP1 | NM_001128834 | 0.06431 | 0.21222 | 0.09646 | 0.30225 | 0.07717 | 0.28939 |
| MED23 | NM_001270521 | 0.29260 | 0.05466 | 0.32154 | 0.01608 | 0.33441 | 0.02572 |
| PSMA5 | NM_001199774 | 0.17685 | 0.11576 | 0.28617 | 0.04502 | 0.38264 | 0.03859 |
| HCFC1R1 | NM_001288668 | 0.06431 | 0.24437 | 0.10611 | 0.21543 | 0.05466 | 0.36013 |
| RNF31 | NM_017999 | 0.10289 | 0.16077 | 0.18971 | 0.15756 | 0.21543 | 0.21865 |
| NTRK2 | NM_001018064 | 0.02572 | 0.15434 | 0.13505 | 0.27653 | 0.17363 | 0.27974 |
| ZIC1 | NM_003412 | 0.06431 | 0.47910 | 0.08039 | 0.17363 | 0.06109 | 0.18650 |
| MME | NM_007287 | 0.01286 | 0.22508 | 0.18971 | 0.17685 | 0.25402 | 0.18650 |
| IFIT3 | NM_001289759 | 0.46945 | 0.17042 | 0.11897 | 0.09646 | 0.10289 | 0.09003 |
| ORC4 | NM_002552 | 0.04180 | 0.35691 | 0.18971 | 0.16720 | 0.21222 | 0.08039 |
| DMD | NM_004006 | 0.05466 | 0.20257 | 0.08039 | 0.32797 | 0.03537 | 0.34727 |
| KRI1 | NM_023008 | 0.10289 | 0.17042 | 0.20900 | 0.17685 | 0.24437 | 0.14469 |
| ZNF813 | NM_001004301 | 0.18971 | 0.01286 | 0.33119 | 0.03537 | 0.44051 | 0.03859 |
| HOXA10 | NM_018951 | 0.10289 | 0.14148 | 0.26688 | 0.04823 | 0.41801 | 0.07074 |
| SEH1L | NM_031216 | 0.53055 | 0.02894 | 0.19293 | 0.00965 | 0.26367 | 0.02251 |
| COMMD9 | NM_014186 | 0.20579 | 0.33441 | 0.16399 | 0.04502 | 0.21865 | 0.08360 |
| GLYATL1 | NM_001220494 | 0.24437 | 0.07717 | 0.19614 | 0.10289 | 0.34727 | 0.08360 |
| RBBP9 | NM_006606 | 0.38264 | 0.00322 | 0.19293 | 0.03537 | 0.34727 | 0.09003 |
| AXIN1 | NM_003502 | 0.10289 | 0.02894 | 0.02251 | 0.36977 | 0.05145 | 0.47588 |
| OBSL1 | NM_015311 | 0.05788 | 0.51125 | 0.00000 | 0.23151 | 0.00000 | 0.25080 |
| HACE1 | NM_020771 | 0.14469 | 0.00322 | 0.44695 | 0.00965 | 0.43730 | 0.01286 |
| MAB21L2 | NM_006439 | 0.02251 | 0.36977 | 0.00322 | 0.26367 | 0.00322 | 0.39228 |
| SARDH | NM_007101 | 0.07717 | 0.48875 | 0.09646 | 0.17042 | 0.06752 | 0.15434 |
| HTR2B | NM_000867 | 0.10289 | 0.24437 | 0.10611 | 0.25723 | 0.09968 | 0.24437 |
| PLS1 | NM_002670 | 0.03859 | 0.29904 | 0.11897 | 0.23473 | 0.10289 | 0.26045 |
| PCP4 | NM_006198 | 0.06431 | 0.17363 | 0.11897 | 0.29582 | 0.15113 | 0.25080 |
| LRP4 | NM_002334 | 0.15434 | 0.16399 | 0.19614 | 0.17363 | 0.26367 | 0.10611 |
| ZNF432 | NM_014650 | 0.06431 | 0.48232 | 0.17685 | 0.07395 | 0.20900 | 0.05145 |
| FAM114A2 | NM_018691 | 0.05788 | 0.28617 | 0.00322 | 0.36656 | 0.05145 | 0.29260 |
| ARMC7 | NM_024585 | 0.08360 | 0.09646 | 0.12540 | 0.18650 | 0.35370 | 0.21222 |
| MFN1 | NM_033540 | 0.07074 | 0.04502 | 0.18971 | 0.20257 | 0.28939 | 0.26045 |
| C12orf74 | NM_001178097 | 0.17363 | 0.02251 | 0.40193 | 0.02894 | 0.41801 | 0.01286 |
| PTCH1 | NM_000264 | 0.10932 | 0.07717 | 0.18006 | 0.17042 | 0.40193 | 0.12219 |
| PELI1 | NM_020651 | 0.25080 | 0.09003 | 0.20579 | 0.16720 | 0.20900 | 0.13826 |
| RPS10 | NM_001203245 | 0.08682 | 0.55949 | 0.01286 | 0.23151 | 0.01929 | 0.15113 |
| ISYNA1 | NM_016368 | 0.12540 | 0.20579 | 0.18971 | 0.17042 | 0.24437 | 0.12862 |
| QRSL1 | NM_018292 | 0.34084 | 0.03215 | 0.31190 | 0.01608 | 0.34727 | 0.01608 |
| PNPLA1 | NM_001145717 | 0.12862 | 0.33441 | 0.09646 | 0.17042 | 0.15756 | 0.17685 |
| CDC42SE2 | NM_001038702 | 0.08360 | 0.08039 | 0.24116 | 0.10611 | 0.34084 | 0.21222 |
| FER1L6 | NM_001039112 | 0.02894 | 0.42122 | 0.00322 | 0.27974 | 0.02894 | 0.30225 |
| BSG | NM_198589 | 0.00000 | 0.23151 | 0.00000 | 0.38907 | 0.00000 | 0.44373 |
| KIF21B | NM_001252100 | 0.27653 | 0.13505 | 0.11254 | 0.13826 | 0.14791 | 0.25402 |
| RNF19A | NM_183419 | 0.13505 | 0.21543 | 0.10611 | 0.23151 | 0.06431 | 0.31190 |
| ZSWIM7 | NM_001042698 | 0.16720 | 0.07717 | 0.19614 | 0.07717 | 0.43087 | 0.11897 |
| KIAA0020 | NM_014878 | 0.29582 | 0.13183 | 0.13183 | 0.20257 | 0.10289 | 0.20257 |
| BCL2A1 | NM_001114735 | 0.18971 | 0.03215 | 0.17685 | 0.16399 | 0.15113 | 0.35370 |
| RBMX | NM_001164803 | 0.00965 | 0.14148 | 0.12540 | 0.29904 | 0.12540 | 0.36656 |
| ARHGEF26 | NM_001251963 | 0.38585 | 0.02894 | 0.35691 | 0.02894 | 0.25402 | 0.01286 |
| MLLT10 | NM_004641 | 0.27653 | 0.07717 | 0.15113 | 0.13505 | 0.14791 | 0.27974 |
| AGAP11 | NM_133447 | 0.12540 | 0.29260 | 0.20900 | 0.18971 | 0.10289 | 0.14791 |
| ZNF260 | NM_001166037 | 0.42444 | 0.16720 | 0.09325 | 0.13505 | 0.09003 | 0.16077 |
| SPPL2C | NM_175882 | 0.18650 | 0.05145 | 0.27010 | 0.05145 | 0.44051 | 0.07074 |
| COL8A1 | NM_001850 | 0.08360 | 0.41801 | 0.01286 | 0.29260 | 0.01286 | 0.25080 |
| RCAN1 | NM_001285392 | 0.15434 | 0.17042 | 0.17685 | 0.22186 | 0.16077 | 0.18650 |
| TNFRSF10A | NM_003844 | 0.25723 | 0.13183 | 0.13505 | 0.12862 | 0.23151 | 0.18650 |
| ERCC5 | NM_000123 | 0.05145 | 0.17042 | 0.13183 | 0.27974 | 0.18971 | 0.25080 |
| MTFR1 | NM_001145839 | 0.29582 | 0.07717 | 0.03859 | 0.28296 | 0.01608 | 0.36334 |
| MSL2 | NM_001145417 | 0.11254 | 0.30547 | 0.19614 | 0.03537 | 0.23794 | 0.18650 |
| PLAC1 | NM_021796 | 0.39228 | 0.03859 | 0.31511 | 0.02572 | 0.28939 | 0.01286 |
| MS4A6E | NM_139249 | 0.33441 | 0.22508 | 0.20257 | 0.06431 | 0.16077 | 0.08682 |
| ATP8B3 | NM_138813 | 0.25080 | 0.53055 | 0.03859 | 0.07717 | 0.02894 | 0.14791 |
| LSP1 | NM_001013253 | 0.44373 | 0.00322 | 0.32154 | 0.00965 | 0.28617 | 0.01286 |
| TNNT1 | NM_003283 | 0.71061 | 0.01286 | 0.19293 | 0.00965 | 0.13826 | 0.01286 |
| C11orf45 | NM_145013 | 0.23794 | 0.07395 | 0.19936 | 0.15756 | 0.24437 | 0.16399 |
| CRIM1 | NM_016441 | 0.29582 | 0.04180 | 0.18006 | 0.15756 | 0.16720 | 0.23473 |
| SLC35G6 | NM_001102614 | 0.13826 | 0.22508 | 0.09003 | 0.28296 | 0.04823 | 0.29260 |
| TRMT12 | NM_017956 | 0.18650 | 0.29260 | 0.22830 | 0.05788 | 0.22508 | 0.08682 |
| ADO | NM_032804 | 0.02572 | 0.14791 | 0.07395 | 0.45338 | 0.08682 | 0.28939 |
| SUPT3H | NM_001261823 | 0.27653 | 0.11576 | 0.16077 | 0.17042 | 0.20900 | 0.14791 |
| POU6F1 | NM_002702 | 0.03537 | 0.05145 | 0.15434 | 0.27653 | 0.28296 | 0.27974 |
| SLC29A1 | NM_001078176 | 0.02894 | 0.16077 | 0.03859 | 0.42444 | 0.05466 | 0.37299 |
| C1QTNF7 | NM_001135170 | 0.04180 | 0.34405 | 0.10611 | 0.22186 | 0.07717 | 0.28939 |
| ST3GAL4 | NM_006278 | 0.12540 | 0.03215 | 0.28617 | 0.04823 | 0.42122 | 0.17042 |
| OSTF1 | NM_012383 | 0.32476 | 0.05145 | 0.03859 | 0.27974 | 0.06431 | 0.32476 |
| ZNF215 | NM_013250 | 0.06431 | 0.14791 | 0.01286 | 0.33762 | 0.16077 | 0.36013 |
| DCAF15 | NM_138353 | 0.29582 | 0.16077 | 0.20579 | 0.07074 | 0.30225 | 0.04823 |
| ZFP41 | NM_173832 | 0.08360 | 0.13826 | 0.24437 | 0.18650 | 0.24437 | 0.18650 |
| GRHL3 | NM_021180 | 0.02894 | 0.23151 | 0.03859 | 0.29260 | 0.07074 | 0.42122 |
| MMS19 | NM_022362 | 0.37299 | 0.05788 | 0.28939 | 0.04502 | 0.24437 | 0.07395 |
| SMAD7 | NM_001190822 | 0.03859 | 0.39871 | 0.08039 | 0.18650 | 0.09325 | 0.28939 |
| BORA | NM_001286746 | 0.10289 | 0.23473 | 0.20257 | 0.13826 | 0.24437 | 0.16399 |
| C6orf1 | NM_001287397 | 0.27653 | 0.13183 | 0.19936 | 0.13505 | 0.25402 | 0.09003 |
| PSME2 | NM_002818 | 0.12540 | 0.32476 | 0.15113 | 0.20257 | 0.07074 | 0.21222 |
| CCL17 | NM_002987 | 0.62701 | 0.20257 | 0.10611 | 0.02894 | 0.05466 | 0.06752 |
| SYT17 | NM_016524 | 0.00000 | 0.57878 | 0.00000 | 0.25723 | 0.00000 | 0.25080 |
| FBXL19 | NM_001282351 | 0.44695 | 0.13183 | 0.20579 | 0.06431 | 0.17363 | 0.06431 |
| C2CD4C | NM_001136263 | 0.33762 | 0.09003 | 0.29904 | 0.08360 | 0.11576 | 0.16399 |
| ALDOC | NM_005165 | 0.04823 | 0.31511 | 0.14469 | 0.18006 | 0.16720 | 0.23473 |
| SDF2L1 | NM_022044 | 0.15434 | 0.23151 | 0.22830 | 0.07395 | 0.23794 | 0.16399 |
| SMIM11 | NM_058182 | 0.27653 | 0.24759 | 0.04180 | 0.20257 | 0.09968 | 0.22186 |
| MRAS | NM_001085049 | 0.13505 | 0.14791 | 0.31190 | 0.03537 | 0.41158 | 0.04823 |
| ZNF469 | NM_001127464 | 0.06431 | 0.16399 | 0.15113 | 0.17685 | 0.38585 | 0.14791 |
| KIAA0391 | NM_001256678 | 0.14469 | 0.06431 | 0.27653 | 0.18328 | 0.28296 | 0.13826 |
| HSFX2 | NM_001164415 | 0.28617 | 0.17685 | 0.20900 | 0.11897 | 0.17363 | 0.12862 |
| PSMA5 | NM_001199773 | 0.00000 | 0.42765 | 0.00000 | 0.53376 | 0.01286 | 0.11897 |
| VNN3 | NM_001291702 | 0.03859 | 0.54662 | 0.03859 | 0.24116 | 0.06431 | 0.16399 |
| WDR47 | NM_014969 | 0.27974 | 0.00965 | 0.26688 | 0.07395 | 0.35370 | 0.10932 |
| SOD1 | NM_000454 | 0.03537 | 0.16077 | 0.19614 | 0.23794 | 0.18971 | 0.27331 |
| TADA2A | NM_001166105 | 0.15434 | 0.02894 | 0.22830 | 0.23473 | 0.25402 | 0.19293 |
| SMARCA2 | NM_001289396 | 0.08360 | 0.60772 | 0.03859 | 0.24437 | 0.01929 | 0.09968 |
| FOXP1 | NM_001012505 | 0.32797 | 0.17042 | 0.22830 | 0.13826 | 0.13826 | 0.09003 |
| CAPG | NM_001747 | 0.03537 | 0.02894 | 0.04180 | 0.44373 | 0.04823 | 0.49518 |
| NAT8 | NM_003960 | 0.02572 | 0.06431 | 0.03859 | 0.43730 | 0.04180 | 0.48553 |
| TRIM52 | NM_032765 | 0.00965 | 0.11576 | 0.17363 | 0.18650 | 0.25080 | 0.35691 |
| SHANK3 | NM_033517 | 0.02251 | 0.49196 | 0.08039 | 0.25723 | 0.05466 | 0.18650 |
| ZNF92 | NM_152626 | 0.10289 | 0.22508 | 0.04180 | 0.33762 | 0.01929 | 0.36656 |
| CELF6 | NM_001172684 | 0.08682 | 0.35691 | 0.17042 | 0.18006 | 0.18650 | 0.11576 |
| MRPS30 | NM_016640 | 0.04180 | 0.32476 | 0.15113 | 0.18650 | 0.30547 | 0.08682 |
| VAMP7 | NM_005638 | 0.15113 | 0.22186 | 0.19936 | 0.17685 | 0.16077 | 0.18650 |
| SLC8A2 | NM_015063 | 0.04180 | 0.16720 | 0.17685 | 0.25402 | 0.20257 | 0.25402 |
| C10orf25 | NM_001039380 | 0.76206 | 0.17042 | 0.08039 | 0.04502 | 0.01608 | 0.02572 |
| EHHADH | NM_001966 | 0.10289 | 0.24759 | 0.13505 | 0.26688 | 0.16077 | 0.18650 |
| TIAF1 | NM_004740 | 0.24437 | 0.06109 | 0.34084 | 0.03215 | 0.39550 | 0.02572 |
| DOCK10 | NM_014689 | 0.12862 | 0.26367 | 0.29582 | 0.05788 | 0.24437 | 0.10932 |
| NTPCR | NM_032324 | 0.10289 | 0.34405 | 0.09325 | 0.21865 | 0.19293 | 0.14791 |
| ABCC12 | NM_033226 | 0.44373 | 0.00643 | 0.34405 | 0.03537 | 0.22508 | 0.04502 |
| SIRT1 | NM_001142498 | 0.25402 | 0.03215 | 0.20257 | 0.03537 | 0.47910 | 0.09968 |
| ZDHHC8 | NM_013373 | 0.03537 | 0.45016 | 0.16077 | 0.17685 | 0.16720 | 0.11254 |
| PUM2 | NM_015317 | 0.12540 | 0.22186 | 0.15756 | 0.10932 | 0.23473 | 0.25402 |
| DCDC2B | NM_001099434 | 0.11576 | 0.05145 | 0.28939 | 0.09646 | 0.44051 | 0.10932 |
| RIOK1 | NM_031480 | 0.13826 | 0.09003 | 0.27331 | 0.12862 | 0.29904 | 0.17363 |
| IFI44L | NM_006820 | 0.14469 | 0.24759 | 0.10611 | 0.19614 | 0.20900 | 0.20257 |
| TP53AIP1 | NM_001195194 | 0.43408 | 0.11254 | 0.12540 | 0.20257 | 0.06431 | 0.16720 |
| TONSL | NM_013432 | 0.44051 | 0.15756 | 0.18971 | 0.00965 | 0.29582 | 0.01286 |
| MAEL | NM_001286377 | 0.38907 | 0.02251 | 0.28617 | 0.03537 | 0.31511 | 0.05788 |
| HCRTR1 | NM_001525 | 0.02894 | 0.14148 | 0.00000 | 0.40193 | 0.02894 | 0.50482 |
| SLC7A10 | NM_019849 | 0.15434 | 0.07717 | 0.18971 | 0.18650 | 0.19614 | 0.30225 |
| C8orf33 | NM_023080 | 0.01929 | 0.21543 | 0.10611 | 0.34727 | 0.08039 | 0.33762 |
| RGS12 | NM_198227 | 0.37942 | 0.14148 | 0.19614 | 0.13826 | 0.14148 | 0.10932 |
| SPTB | NM_001024858 | 0.03537 | 0.44373 | 0.02572 | 0.29904 | 0.04180 | 0.26045 |
| LYL1 | NM_005583 | 0.00000 | 0.46302 | 0.10611 | 0.24116 | 0.15756 | 0.14148 |
| CPA1 | NM_001868 | 0.15756 | 0.19614 | 0.19614 | 0.13826 | 0.30225 | 0.11897 |
| C12orf4 | NM_020374 | 0.41801 | 0.00322 | 0.35370 | 0.02572 | 0.28296 | 0.02572 |
| NFIX | NM_001271043 | 0.32476 | 0.07717 | 0.24759 | 0.04823 | 0.36013 | 0.05466 |

### Pharmaceutical Formulations and Administration

Also described herein are pharmaceutical formulations that can contain an amount, effective amount, and/or least effective amount, and/or therapeutically effective amount of one or more of the small molecules, RNAi therapeutics, vectors, recombinant polypeptides, gene editing systems, conjugated-antibodies, or engineered cells as described above, or a combination thereof (which are also referred to as the primary active agent or ingredient elsewhere herein) described in greater detail elsewhere herein a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having a Type-2 Diabetes or a symptom thereof. In some embodiments, the subject in need thereof has or is suspected of having, a metabolic disease or disorder, insulin resistance, or glucose intolerance, or a combination thereof. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects.

The effective amount, least effective amount, and/or therapeutically effective amount of the primary and optional secondary active agent described elsewhere herein contained in the pharmaceutical formulation can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, μg, mg, or g or be any numerical value with any of these ranges.

In some embodiments, the effective amount, least effective amount, and/or therapeutically effective amount can be an effective concentration, least effective concentration, and/or therapeutically effective concentration, which can each range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value with any of these ranges.

In other embodiments, the effective amount, least effective amount, and/or therapeutically effective amount of the primary and optional secondary active agent can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In some embodiments, the primary and/or the optional secondary active agent present in the pharmaceutical formulation can range from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation.

In some embodiments where a cell population is present in the pharmaceutical formulation (e.g., as a primary and/or or secondary active agent), the effective amount of cells can range from about 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL.

In some embodiments, the amount or effective amount, particularly where an infective particle is being delivered (e.g. a virus particle having the primary or secondary agent as a cargo), the effective amount of virus particles can be expressed as a titer (plaque forming units per unit of volume) or as a MOI (multiplicity of infection). In some embodiments, the effective amount can be 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more.

In some embodiments, the amount or effective amount of the one or more of the active agent(s) described herein contained in the pharmaceutical formulation can range from about 1 μg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total secondary active agent in the pharmaceutical formulation. In additional embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wlkins, Baltimore, M D, 2000, and “Pharmaceutical dosage forms and drug delivery systems”, 6th Edition, Ansel et al., (Media, PA: Wlliams and Wlkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g. micronized) compound or salt or solvate thereof, is defined by a D50 value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, manitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein can be part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

### Administration of the Pharmaceutical Formulations

The pharmaceutical formulations or dosage forms thereof described herein can be administered one or more times hourly, daily, monthly, or yearly (e.g. 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g. 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g. within seconds or just a few minutes apart), where the intent is that the formulations be administered together at the same time.

### Viral Vector Formulation, Dosage, and Delivery

Compositions of the invention may be formulated for delivery to human subjects, as well as to animals for veterinary purposes (e.g. livestock (cattle, pigs, others)), and other non-human mammalian subjects. The dosage of the formulation can be measured or calculated as viral particles or as genome copies (“GC”)/viral genomes (“vg”). Any method known in the art can be used to determine the genome copy (GC) number of the viral compositions of the invention. In one example embodiment, the viral compositions can be formulated in dosage units to contain an amount of viral vectors that is in the range of about 1.0×10⁹ GC to about 1.0×10¹⁵ GC (to treat an average subject of 70 kg in body weight), and preferably 1.0×10¹² GC to 1.0×10¹⁴ GC for a human patient. Preferably, the dose of virus in the formulation is 1.0×10⁹ GC, 5.0×10⁹ GC, 1.0×10¹⁰ GC, 5.0×10¹⁰ GC, 1.0×10¹¹GC, 5.0×10¹¹ GC, 1.0×10¹² GC, 5.0×10¹² GC, or 1.0×10¹³ GC, 5.0×10¹³ GC, 1.0×10¹⁴ GC, 5.0×10¹⁴ GC, or 1.0×10¹⁵ GC.

The viral vectors can be formulated in a conventional manner using one or more physiologically acceptable carriers or excipients. The viral vectors may be formulated for parenteral administration by injection (e.g. by bolus injection or continuous infusion). Formulations for injection may be presented in unit dosage form (e.g. in ampoules or in multi-dose containers) with an added preservative. The viral compositions may take such forms as suspensions, solutions, or emulsions in oily or aqueous vehicles, and may contain formulatory agents such as suspending, stabilizing, or dispersing agents. Liquid preparations of the viral vector formulations may be prepared by conventional means with pharmaceutically acceptable additives such as suspending agents (e.g. sorbitol syrup, cellulose derivatives or hydrogenated edible fats), emulsifying agents (e.g. lecithin or acacia), non-aqueous vehicles (e.g. almond oil, oily esters, ethyl alcohol or fractionated vegetable oils), and preservatives (e.g. methyl or propyl-p-hydroxybenzoates or sorbic acid). The preparations may also contain buffer salts. Alternatively, the compositions may be in powder form for constitution with a suitable vehicle (e.g. sterile pyrogen-free water) before use.

### Recombinant Protein Formulation, Dosage, and Delivery

In one example embodiment, virus like particles (VLPs) are used to facilitate intracellular recombinant protein therapy (see, e.g., WO2020252455A1, U.S. Ser. No. 10/577,397B2). In certain embodiments, VLPs include a Gag-COBLL1 fusion protein. The Gag-COBLL1 fusion protein may include a matrix protein, a capsid protein, and/or a nucleocapsid protein covalently linked to COBLL1. In certain embodiments, the VLPs include a membrane comprising a phospholipid bilayer with one or more human endogenous retrovirus (HERV) derived ENV/glycoprotein(s) on the external side; a HERV-derived GAG protein in the VLP core, and a COBLL1 fusion protein on the inside of the membrane, wherein COBLL1 is fused to a human-endogenous GAG or other plasma membrane recruitment domain (see, e.g., WO2020252455A1). Fusion proteins can be obtained using standard recombinant protein technology.

In one example embodiment, cell-penetrating peptides (CPPs) are used to facilitate intracellular recombinant protein therapy (see, e.g., Dinca A, Chien W-M, Chin M T. Intracellular Delivery of Proteins with Cell-Penetrating Peptides for Therapeutic Uses in Human Disease. International Journal of Molecular Sciences. 2016; 17(2):263). In certain embodiments, cell-penetrating peptides can be conjugated to COBLL1, for example, using standard recombinant protein technology. In certain embodiments, cell-penetrating peptides can be concurrently delivered with recombinant COBLL1.

In one example embodiment, nanocarriers are used to facilitate intracellular recombinant protein therapy (see, e.g., Lee Y W, Luther D C, Kretzmann J A, Burden A, Jeon T, Zhai S, Rotello V M. Protein Delivery into the Cell Cytosol using Non-Viral Nanocarriers. Theranostics 2019; 9(11):3280-3292). Non-limiting nanocarriers include, but are not limited to nanoparticles (e.g., silica, gold), polymers, lipid based (e.g., cationic lipid within a polymer shell, lipid-like nanoparticles).

The pharmaceutical composition of the invention may be administered locally or systemically. In a preferred embodiment, the pharmaceutical composition is administered near the tissue whose cells are to be transduced. In a particular embodiment, the pharmaceutical composition of the invention is administered locally to the subcutaneous adipose tissue, which is composed of varying amounts of the two different types of adipose tissue: white adipose tissue (WAT) that stores energy in the form of triacylglycerol (TAG) and brown adipose tissue (BAT) that dissipates energy as heat, “burning” fatty acids to maintain body temperature. In one example embodiment, the pharmaceutical composition of the invention is administered in the white adipose tissue (WAT) and/or in the brown adipose tissue (BAT) by intra-WAT or intra-BAT injection. In another preferred embodiment, the pharmaceutical composition of the invention is administered systemically.

The “adeno-associated virus” (AAV) can be formulated with a physiologically acceptable carrier for use in gene transfer and gene therapy applications. The dosage of the formulation can be measured or calculated as viral particles or as genome copies (“GC”)/viral genomes (“vg”). Any method known in the art can be used to determine the genome copy (GC) number of the viral compositions of the invention. One method for performing AAV GC number titration is as follows: purified AAV vector samples are first treated with DNase to eliminate un-encapsulated AAV genome DNA or contaminating plasmid DNA from the production process. The DNase resistant particles are then subjected to heat treatment to release the genome from the capsid. The released genomes are then quantitated by real-time PCR using primer/probe sets targeting specific region of the viral genome.

In any of the described methods the one or more vectors may be comprised in a delivery system. In any of the described methods the vectors may be delivered via liposomes, particles (e.g., nanoparticles), exosomes, microvesicles, a gene-gun. In any of the described methods viral vectors may be delivered by transduction of viral particles. The delivery systems may be administered systemically or by localized administration (e.g., direct injection). The term “systemically administered” and “systemic administration”, as used herein, means that the polynucleotides, vectors, polypeptides, or pharmaceutical compositions of the invention are administered to a subject in a non-localized manner. The systemic administration of the polynucleotides, vectors, polypeptides, or pharmaceutical compositions of the invention may reach several organs or tissues throughout the body of the subject or may reach specific organs or tissues of the subject. For example, the intravenous administration of a pharmaceutical composition of the invention may result in the transduction of more than one tissue or organ in a subject. The term “transduce” or “transduction”, as used herein, refers to the process whereby a foreign nucleotide sequence is introduced into a cell via a viral vector. The term “transfection”, as used herein, refers to the introduction of DNA into a recipient eukaryotic cell.

Recombinant protein compositions described herein may be administered systemically (e.g., intravenously) or administered locally to adipose tissue (e.g., injection). In preferred embodiments, the recombinant protein compositions are administered with an appropriate carrier to be administered to a mammal, especially a human, preferably a pharmaceutically acceptable composition. A “pharmaceutically acceptable composition” refers to a non-toxic semisolid, liquid, or aerosolized filler, diluent, encapsulating material, colloidal suspension or formulation auxiliary of any type. Preferably, this composition is suitable for injection. These may be in particular isotonic, sterile, saline solutions (monosodium or disodium phosphate, sodium, potassium, calcium or magnesium chloride and similar solutions or mixtures of such salts), or dry, especially freeze-dried compositions which upon addition, depending on the case, of sterilized water or physiological saline, permit the constitution of injectable solutions.

### CRISPR-Cas Delivery

The CRISPR-Cas systems disclosed herein may be delivered using vectors comprising polynucleotides encoding the Cas polypeptide and the guide molecule. For HDR based embodiments, the donor template may also be encoded on a vector. Vectors, dosages, and adipocyte-specific configurations suitable for delivery of these components include those discussed above.

The vector(s) can include regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s). By simple arithmetic and well-established cloning protocols and the teachings in this disclosure one skilled in the art can readily practice the invention as to the RNA(s) for a suitable exemplary vector such as AAV, and a suitable promoter such as the U6 promoter. For example, the packaging limit of AAV is ˜4.7 kb. The length of a single U6-gRNA (plus restriction sites for cloning) is 361 bp. Therefore, the skilled person can readily fit about 12-16, e.g., 13 U6-gRNA cassettes in a single vector. This can be assembled by any suitable means, such as a golden gate strategy used for TALE assembly (genome-engineering.org/taleffectors/). The skilled person can also use a tandem guide strategy to increase the number of U6-gRNAs by approximately 1.5 times, e.g., to increase from 12-16, e.g., 13 to approximately 18-24, e.g., about 19 U6-gRNAs. Therefore, one skilled in the art can readily reach approximately 18-24, e.g., about 19 promoter-RNAs, e.g., U6-gRNAs in a single vector, e.g., an AAV vector. A further means for increasing the number of promoters and RNAs in a vector is to use a single promoter (e.g., U6) to express an array of RNAs separated by cleavable sequences. And an even further means for increasing the number of promoter-RNAs in a vector is to express an array of promoter-RNAs separated by cleavable sequences in the intron of a coding sequence or gene; and, in this instance, it is advantageous to use a polymerase II promoter, which can have increased expression and enable the transcription of long RNA in a tissue specific manner. (See, e.g., Chung K H, Hart C C, Al-Bassam S, et al. Polycistronic RNA polymerase II expression vectors for RNA interference based on BIC/miR-155. Nucleic Acids Res. 2006; 34(7):e53). In an advantageous embodiment, AAV may package U6 tandem gRNA targeting up to about 50 genes. Accordingly, from the knowledge in the art and the teachings in this disclosure the skilled person can readily make and use vector(s), e.g., a single vector, expressing multiple RNAs or guides under the control or operatively or functionally linked to one or more promoters, especially as to the numbers of RNAs or guides discussed herein, without any undue experimentation.

The Cas polypeptide and guide molecule (and donor) may also be delivered as a pre-formed ribonucleoprotein complex (RNP). Delivery methods for delivery RNPs include virus like particles, cell-penetrating peptides, and nanocarriers discussed above.

Delivery mechanisms for CRISPRa systems include virus like particles, cell-penetrating peptides, and nanocarriers discussed above for CRISPR-Cas systems.

### Base Editing Delivery

Base editing systems may deliver on one or more vectors encoding the Cas-nucleobase deaminase and guide sequence. Vector systems suitable for this purpose includes those discussed above. Alternatively, base editing systems may be delivered as pre-complex Ribonucleoprotein complex (RNP. Systems for delving RNPs include the protein delivery systems: virus like particles; cell-penetrating peptides; and nanocarriers, discuss above.

A further example method for delivery of base-editing systems may include use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Diagnostic and Theranostic Methods

### Methods of Predicting Response to Immunotherapy

Provided herein are methods for determining if a subject will be responsive to an immunotherapy comprising detecting expression of one or more biomarkers from Table 2. Table 2 includes the list of 576 candidate genes and Gene Refseq accession numbers for each candidate gene. The candidate genes were enriched in the top 1% across at least two screening replicates for the MAGeCK (Table 1) and FDR (Table 3) analyses in either the acute or chronic screens. The biomarkers in Table 2 were identified as having increased expression in tumors that did not respond to immunotherapy. As used herein, the term “responder” refers to a subject that receives a benefit from an immunotherapy. As used herein, the term “non-responder” refers to a subject that does not receive a benefit from an immunotherapy. Responders and non-responders can also be determined based on progression (non-responder) or regression (responder) of a tumor in response to an immunotherapy. Responders and non-responders can be based on radiologic tumor assessments of progression or regression. Responders and non-responders can also be determined based on RECIST criteria: complete response (CR) and partial response (PR) for responders, or stable disease (SD) and progressive disease (PD) for non-responders (see, e.g., Eisenhauer, E. A. et al., 2009, New response evaluation criteria in solid tumours: revised RECIST guideline (version 1.1). Eur J Cancer 45, 228-247). “Immunotherapy” in this context is as defined at to above. “Biomarkers” in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein. By means of further explanation and without limitation the term “diagnosis” generally refers to the process or act of recognizing, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such. In the context of the embodiments disclosed herein, the prognosis may refer to whether the subject will be a responder or non-responder.

Applicants have identified that MCL1, BCL2A1, JUNB, B3GNT2 increase in expression in response to an immunotherapy. In an example embodiment, MCL1, BCL2A1, JUNB, B3GNT2 and/or alternate immune evasion targets in Table 2 increase in expression upon treatment with an immunotherapy and can be used to monitor the efficacy of an immunotherapy. The increased expression may indicate that the tumor is evading an anti-tumor immune response and the tumor may not respond to further immunotherapy. Thus, in certain embodiments, one or more of the targets is used as a biomarker for monitoring the efficacy of an immunotherapy and guiding further treatment as described herein. An increase in expression can be determined by comparing expression from samples obtained from a subject before and during treatment.

In an example embodiment, is disclosed a method determining if a subject is an immunotherapy responder and non-responder comprises detecting in a tumor obtained from the subject the expression or activity of 576 genes, of 500 to 575 genes, of 400 to 500 genes, of 300 to 400 genes, of 200 to 300 genes, of 100 to 200 genes, of 50 to 100 genes, of 25 to 50 genes, of 10 to 25 genes, of 5 to 10 genes or of 1 to 5 genes selected from candidate genes in Table 2, wherein if the expression of the genes is higher than a reference value the subject is an immunotherapy non-responder and if the one or more genes is lower than a reference value then the subject is an immunotherapy responder; and treating the subject, wherein if the subject is a responder, administering an immunotherapy. In preferred embodiments, one or more of B3GNT2, MCL1, BCL2A, and/or JUNB is detected, wherein elevated expression relative to a reference value indicates the subject is an immunotherapy non-responder.

The reference value may be indicated inter alia as an absolute value, range or statistics, or may be indicated relative to a suitable control subject or subject population (such as, e.g., relative to a general, normal or healthy subject or subject population). Hence, the probability, that a subject will be an immunotherapy responder or non-responder may be advantageously indicated as increased or decreased, or as fold-increased or fold-decreased relative to a suitable control subject or subject population. Methods may rely on comparing the quantity of biomarkers, or gene or gene product signatures measured in samples from patients with reference values, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterized by a particular diagnosis, prediction and/or prognosis of responding to an immunotherapy or not responding to immunotherapy (i.e., for whom said diagnosis, prediction and/or prognosis of the disease or condition holds true). Such population may comprise without limitation 2 or more, 10 or more, 100 or more, or even several hundred or more individuals.

In one example embodiment, a reference value can be determined by the evaluation of the expression of candidate genes listed in Table 2 in an annotated database of tumor samples that provides gene expression and clinical outcome. For example, by analyzing the transcriptomes of 310 patients collected prior to immune checkpoint blockade therapy and determining whether the subject was responsive or non-responsive a reference value can be established. Example databases have been described (see, e.g., Auslander et al., 2018; Braun et al., 2020; Gide et al., 2019; Hugo et al., 2016; Pender et al., 2021; Riaz et al., 2017 and the Cancer Genome Atlas website (www.genome.gov/Funded-Programs-Projects/Cancer-Genome-Atlas)).

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR—), Youden index, or similar.

### Theranostic Methods

Methods of determining if a subject is an immunotherapy responder or non-responder may also be integrated into method of treatments to guide appropriate treatment regimens. In one example embodiment, a method of treating cancer comprising determining if the subject is an immunotherapy non-responder or an immunotherapy responder as described above, and treating the subject with one or more of the methods of enhancing anti-tumor immunity described in the sections above, if the subject is an immune non-responder, and treating the subject with an immunotherapy if the subject is an immunotherapy responder.

### Detection of Biomarkers

In one embodiment, the signature genes, biomarkers, and/or cells expressing biomarkers may be detected or isolated by immunofluorescence, immunohistochemistry (IHC), fluorescence activated cell sorting (FACS), mass spectrometry (MS), mass cytometry (CyTOF), sequencing, WGS (described herein), WES (described herein), RNA-seq, single cell RNA-seq (described herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. Detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25). In certain embodiments, cancer is diagnosed, prognosed, or monitored. For example, a tissue sample may be obtained and analyzed for specific cell markers (IHC) or specific transcripts (e.g., RNA-FISH). In one embodiment, tumor cells are stained for cell subtype specific signature genes (e.g., responder or non-responder). In one embodiment, the cells are fixed. In another embodiment, the cells are formalin fixed and paraffin embedded. Not being bound by a theory, the presence of the tumor subtypes indicate outcome and personalized treatments.

The present invention also may comprise a kit with a detection reagent that binds to one or more biomarkers or can be used to detect one or more biomarkers.

### MS Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affibodies, nanobodies, ankyrins, domain antibodies, alternative antibody scaffolds (e.g. diabodies etc.) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992).

In certain embodiments, a subject can be categorized based on signature genes or gene programs expressed by a tissue sample obtained from the subject. In certain embodiments, the tissue sample is analyzed by bulk sequencing. In certain embodiments, subtypes can be determined by determining the percentage of specific cell subtypes expressing the identified interacting genetic variants in the sample that contribute to the phenotype. In certain embodiments, gene expression associated with the cells are determined from bulk sequencing reads by deconvolution of the sample. For example, deconvoluting bulk gene expression data obtained from a tumor containing both malignant and non-malignant cells can include defining the relative frequency of a set of cell types in the tumor from the bulk gene expression data using cell type specific gene expression (e.g., cell types may be T cells, fibroblasts, macrophages, mast cells, B/plasma cells, endothelial cells, myocytes and dendritic cells); and defining a linear relationship between the frequency of the non-malignant cell types and the expression of a set of genes, wherein the set of genes comprises genes highly expressed by malignant cells and at most two non-malignant cell types, wherein the set of genes are derived from gene expression analysis of single cells in the tumor or the same tumor type, and wherein the residual of the linear relationship defines the malignant cell-specific (MCS) expression profile (see, e.g., WO 2018/191553; and Puram et al., Cell. 2017 Dec. 14; 171(7):1611-1624.e24).

### Sequencing

In certain embodiments, sequencing is used to identify expression of genes or transcriptomes in single cells. In certain embodiments, sequencing comprises high-throughput (formerly “next-generation”) technologies to generate sequencing reads. Methods for constructing sequencing libraries are known in the art (see, e.g., Head et al., Library construction for next-generation sequencing: Overviews and challenges. Biotechniques. 2014; 56(2): 61-77). A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags. In certain embodiments, the library members (e.g., cDNA) may include sequencing adaptors that are compatible with use in, e.g., Illumina's reversible terminator method, long read nanopore sequencing, Roche's pyrosequencing method (454), Life Technologies' sequencing by ligation (the SOLID platform) or Life Technologies' Ion Torrent platform. Examples of such methods are described in the following references: Margulies et al (Nature 2005 437: 376-80); Schneider and Dekker (Nat Biotechnol. 2012 Apr. 10; 30(4):326-8); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol. Biol. 2009; 553:79-108); Appleby et al (Methods Mol. Biol. 2009; 513:19-39); and Morozova et al (Genomics. 2008 92:255-64), which are incorporated by reference for the general descriptions of the methods and the particular steps of the methods, including all starting products, reagents, and final products for each of the steps.

As used herein the term “transcriptome” refers to the set of transcript molecules. In some embodiments, transcript refers to RNA molecules, e.g., messenger RNA (mRNA) molecules, small interfering RNA (siRNA) molecules, transfer RNA (tRNA) molecules, ribosomal RNA (rRNA) molecules, and complimentary sequences, e.g., cDNA molecules. In some embodiments, a transcriptome refers to a set of mRNA molecules. In some embodiments, a transcriptome refers to a set of cDNA molecules. In some embodiments, a transcriptome refers to one or more of mRNA molecules, siRNA molecules, tRNA molecules, rRNA molecules, in a sample, for example, a single cell or a population of cells. In some embodiments, a transcriptome refers to cDNA generated from one or more of mRNA molecules, siRNA molecules, tRNA molecules, rRNA molecules, in a sample, for example, a single cell or a population of cells. In some embodiments, a transcriptome refers to 25%, 50%, 55, 60, 65, 70, 75, 80, 85, 90, 91, 92, 93, 94, 95, 96, 97, 98, 99, 99.9, or 100% of transcripts from a single cell or a population of cells. In some embodiments, transcriptome not only refers to the species of transcripts, such as mRNA species, but also the amount of each species in the sample. In some embodiments, a transcriptome includes each mRNA molecule in the sample, such as all the mRNA molecules in a single cell.

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p666-673, 2012).

In certain embodiments, the present invention involves single cell RNA sequencing (scRNA-seq). In certain embodiments, the invention involves plate based single cell RNA sequencing (see, e.g., Picelli, S. et al., 2014, “Full-length RNA-seq from single cells using Smart-seq2” Nature protocols 9, 171-181, doi: 10.1038/nprot.2014.006).

In certain embodiments, the invention involves high-throughput single-cell RNA-seq where the RNAs from different cells are tagged individually, allowing a single library to be created while retaining the cell identity of each read. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International Patent Application No. PCT/US2016/059239, published as WO2017164936 on Sep. 28, 2017; International Patent Application No. PCT/US2018/060860, published as WO/2019/094984 on May 16, 2019; International Patent Application No. PCT/US2019/055894, published as WO/2020/077236 on Apr. 16, 2020; and Drokhlyansky, et al., “The enteric nervous system of the human and mouse colon at a single-cell resolution,” bioRxiv 746743; doi: doi.org/10.1101/746743, which are herein incorporated by reference in their entirety.

### Methods for Screening for Agents Capable of Decreasing Poly-LacNac on Tumor Surface

A further aspect of the invention relates to a method for identifying an agent capable of decreasing poly-lacnac on the tumor surface, comprising: contacting a population of tumor cells having upregulated B3GNT2 with an agent; and detecting binding of one or more proteins to the tumor cells selected from the group consisting of CD2, 4-1BB, TREML2 (TLT2), NKG2D, and an antibody specific for an HLA class I bound tumor antigen, wherein increased binding indicates reduced poly-LacNAc. Applicants further describe changes in binding of these proteins to cells dependent upon glycosylation (see, examples). In a specific embodiment, the assay uses one or more of the proteins, such that the proteins are labeled with a detectable marker. Thus, in certain embodiments, the screen can be a high throughput assay. The detectable marker can be any fluorescent marker known in the art.

Further embodiments are illustrated in the following Examples, which are given for illustrative purposes only and are not intended to limit the scope of the invention.

### EXAMPLES

### Example 1—Identification of Novel Checkpoint Inhibitor Targets Using a CRISPR Activation Screen

Applicants first established a T cell cytotoxicity assay for measuring immunotherapy resistance. Applicants transduced human primary CD4⁺ and CD8⁺ T cells with a T cell receptor (TCR) specific for the NY-ESO-1 antigen (NY-ESO-1:157-165 epitope) presented in an HLA-A*02-restricted manner (ESO T cells) (Robbins et al., 2008). When A375 (NY-ESO-1⁺, HLA-A2⁺) human melanoma cells were exposed to ESO T cells, Applicants observed cytotoxicity that was specific to the presence of the NY-ESO-1 antigen and NY-ESO-1 TCR (FIG. 1a-c). Cytotoxicity correlated with the effector to target (E:T) ratio (FIG. 1b-c). Applicants then transduced A375 cells with a genome-scale CRISPRa single-guide RNA (sgRNA) library consisting of 70,290 sgRNAs targeting every coding isoform from the RefSeq database (23,430 isoforms) to systematically identify genes that enable tumors to evade T cell killing upon upregulation (Konermann et al., 2015). Applicants tested two T cell exposure strategies: acute (E:T ratio of 3 for 18 hours) and chronic (E:T ratio of 2 for 3 days with 3 rounds of screening selection), in independent screens (FIG. 2a). Applicants deep sequenced the sgRNA library distribution in the surviving cells with or without ESO T cell exposure (FIG. 1d,g). In the chronic exposure screen, Applicants observed that the skew of the distribution increased after each round of screening selection (FIG. 1e,g).

Applicants performed MAGeCK (Li et al., 2014) and FDR analyses to identify candidate genes that were enriched in cells cultured with ESO T cells relative to control (FIG. 2b, and Tables 1-3). Pathway analysis on 576 genes prioritized by MAGeCK (top 1% of multiple screening replicates combining the acute and chronic screens) revealed pathways were significantly enriched (FDR<0.05) within these top candidates, including some that have been previously shown to be important for tumor immune evasion, such as lipopolysaccharide response, extrinsic apoptosis signaling, NF-κB activation, JAK-STAT signaling, and antigen presentation (Lawson et al., 2020; Manguso et al., 2017; Pan et al., 2018; Patel et al., 2017; Vredevoogd et al., 2019) (FIG. 2c and Table 4). This analysis also highlighted pathways with previously underappreciated roles in regulating tumor response to T cell cytotoxicity, including glycosaminoglycan metabolism and Wnt signaling, perhaps due to the different screening perturbation (FIG. 2c and Table 4). To assess whether expression of these candidate genes was associated with local immune cytolytic activity in patient tumors (quantified using granzyme A and perforin bulk transcriptome data) (Patel et al., 2017; Rooney et al., 2015), Applicants analyzed gene expression of 33 tumor types from The Cancer Genome Atlas (TCGA). Applicants found that expression of many candidate genes positively correlated (FDR<0.05) with cytolytic activity across tumor types, similar to known immunotherapy resistance mediators, as high cytolytic activity selects for emergence of evading tumor subclones (FIG. 2d, FIG. 3a-d) (Rooney et al., 2015). MAGeCK analysis for genes that generally affect A375 cell growth in the absence of ESO T cell co-culture showed that the MYC pathway governs growth, with MYC and its antagonist MXI1 as the top genes promoting growth and death respectively (FIG. 3e-f). Out of 576 candidate genes, 5 generally drive A375 cell growth and 19 repress it (ranking in the top 1%).

Applicants sought to evaluate whether expression of candidate genes is associated with clinical outcome by analyzing 310 patient transcriptomes collected prior to immune checkpoint blockade therapy (Auslander et al., 2018; Braun et al., 2020; Gide et al., 2019; Hugo et al., 2016; Pender et al., 2021; Riaz et al., 2017). In this analysis, Applicants found expression of candidate genes was significantly higher in non-responders (FIG. 2e).

FIG. 2f. shows the cell survival of A375 cells transduced with ORFs encoding candidate genes against ESO T cell cytotoxicity at different effector to target (E:T) ratios. Cell survival was measured using a luminescent cell viability assay and normalized to paired control cells that were not cultured with T cells. T cells were derived from donors used in the CRISPRa screen.

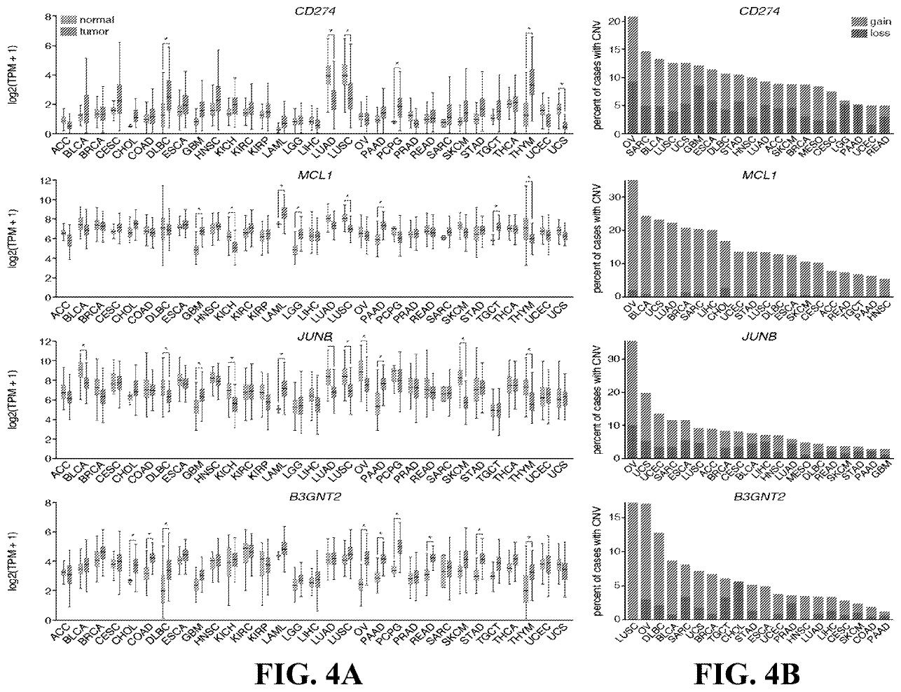

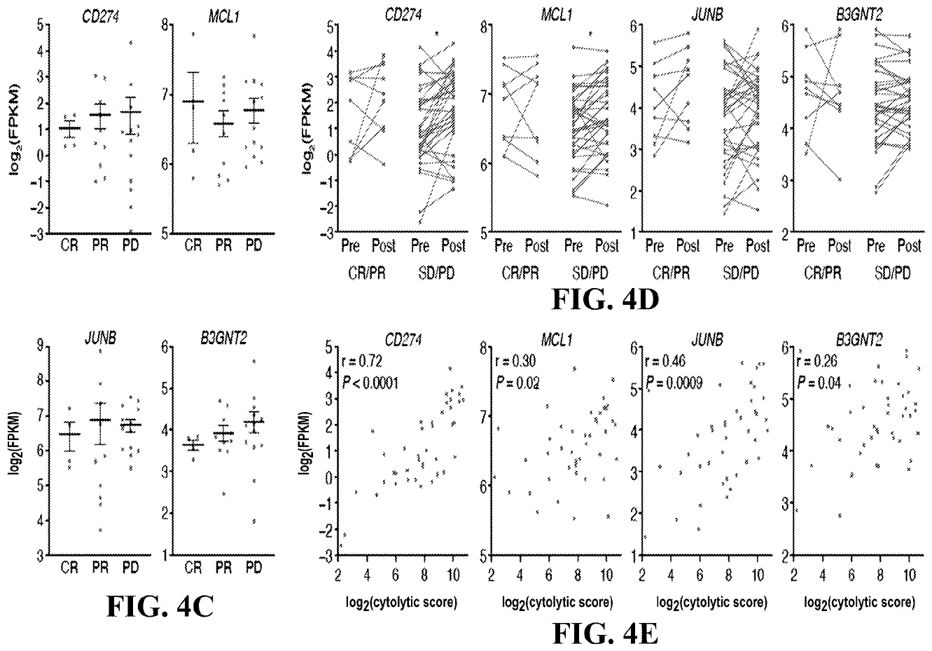

To narrow the focus for further analysis, Applicants selected the two most enriched genes from each screening strategy: CD274 and MCL1 from the acute screen, and JUNB and B3GNT2 from the chronic exposure screen (FIG. 1j-k). Of these four candidates, CD274 (PD-L1) is known to play a role in immune evasion, and it is currently the focus of immune checkpoint blockade therapies, supporting the design of this study (Reck et al., 2016). Applicants validated the four candidate genes by individually expressing three sgRNAs targeting each gene in A375 cells. For each candidate gene, at least two sgRNAs significantly increased survival against ESO T cells (P<0.05; FIG. 4g-h), verifying the screening results. For JUNB and B3GNT2, sgRNAs that produced higher target gene expression were more enriched in the screen and conferred more resistance, suggesting that these genes are dose-dependent (FIG. 4g-h). Overexpression of ORFs encoding each of the four candidate genes increased survival against T cell cytotoxicity, excluding the possibility of potential CRISPRa off-target genes contributing to resistance (FIG. 5e).

Applicants sought to assess the clinical relevance of candidate genes by examining patient tumor samples from TCGA. Applicants found that expression of B3GNT2 was significantly higher than matched normal samples for 9 out of 31 types of cancer (FIG. 6a). Copy number gain of MCL1 and B3GNT2 occurred more frequently than losses, in 95% and 81% of total cases respectively (FIG. 4b). In melanoma patients treated with PD-1 immunotherapy, higher B3GNT2 expression was associated with poorer clinical response (FIG. 4c) (Hugo et al., 2016). A different patient cohort showed that expression of CD274 and MCL1 significantly increased after treatment in patients that did not respond to immunotherapy (FIG. 4d) (Riaz et al., 2017). Expression of all four candidate genes significantly correlated with cytolytic activity (FIG. 4e) (Riaz et al., 2017). Together, these results suggest that the effects of MCL1 and B3GNT2 overexpression on immune evasion may be more common and clinically relevant than that of JUNB.

Next, Applicants evaluated whether the screening results were generalizable to other contexts by testing different T cells and co-culture conditions. Overexpression of all candidate genes in A375 cells conferred resistance against ESO T cells from two additional donors that were not used in the CRISPRa screens (FIG. 6a). Applicants verified that candidate gene overexpression promoted resistance over time in an alternative T cell cytotoxicity assay based on secreted Gaussia luciferase (FIG. 6b). In the absence of T cell cytotoxicity, upregulation of candidate genes did not consistently affect cell proliferation across the two cytotoxicity assays (FIG. 6c-d). Applicants investigated how expression level affects resistance by titrating the expression of candidate genes and found that expression correlated with resistance at lower levels of induction (FIG. 6e). The expression threshold above which Applicants observed increased resistance corresponded to the baseline expression of 5-31% of cell lines from the Cancer Cell Line Encyclopedia (Barretina et al., 2012), which suggested that the expression threshold for resistance is physiologically relevant (FIG. 6f). Applicants tested whether resistance conferred by candidate genes was specific to CD4⁺ or CD8⁺ T cells and found that candidate genes conferred resistance to both types of T cells (FIG. 6g). To determine whether resistance against T cells expressing the NY-ESO-1 TCR also applies to those expressing chimeric antigen receptors (CARs), Applicants introduced candidate gene ORFs into A375 (AXL⁺) cells and co-cultured the cells with AXL-targeting CAR T cells (Cho et al., 2018). Overexpression of each of the four candidate genes increased resistance against AXL-targeting CAR T cell cytotoxicity (FIG. 6h). These results show that candidate genes confer resistance against different cytotoxic T cells and across co-culture conditions.

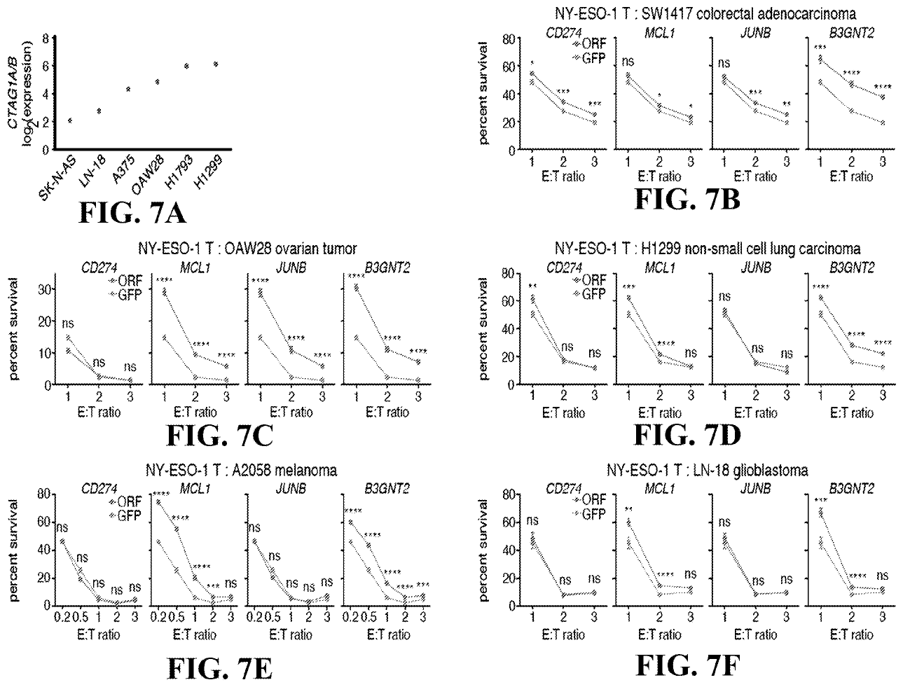

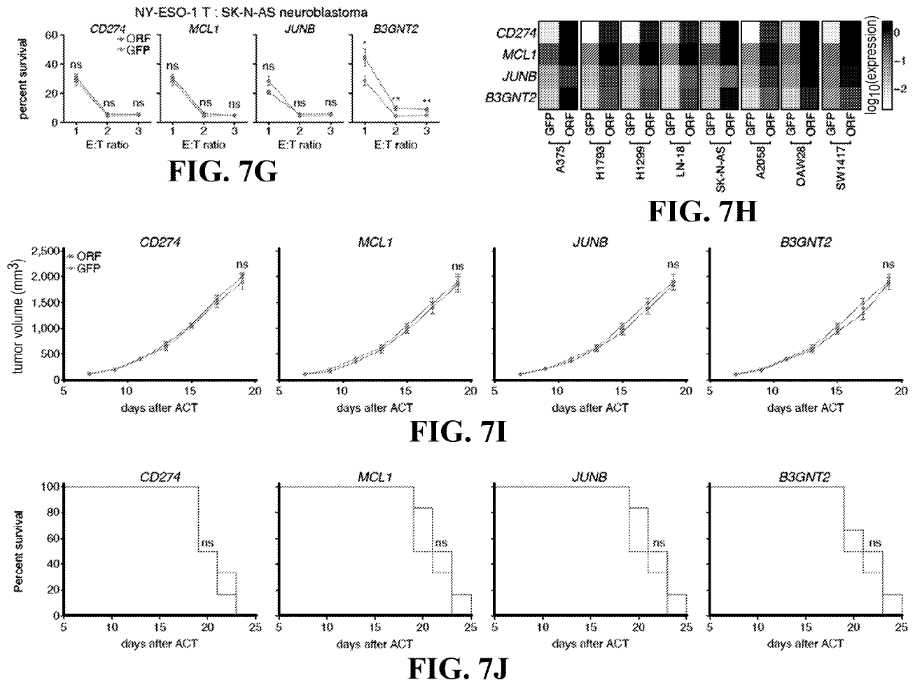

Next, Applicants evaluated whether the screening results were generalizable to other types of cancers. Applicants assayed candidate genes in 7 additional cancer cell lines derived from 5 additional tissues [H1793 (NY-ESO-1⁺, HLA-A2⁻) and H1299 (NY-ESO-1⁺, HLA-A2⁻) non-small cell lung carcinomas, SW1417 (NY-ESO-1⁻, HLA-A2⁻) colorectal adenocarcinoma, OAW28 (NY-ESO-1⁺, HLA-A2⁻) ovarian cystadenocarcinoma, A2058 (NY-ESO-1⁻, HLA-A2⁻) melanoma, LN-18 (NY-ESO-1⁺, HLA-A2⁺) glioblastoma, and SK-N-AS (NY-ESO-1⁺, HLA-A2⁻) neuroblastoma]. Five of these cell lines expressed the NY-ESO-1 antigen endogenously, at varying levels (FIG. 7a), and those that did not naturally express HLA-A2 or NY-ESO-1 were transduced with the appropriate expression vectors. Applicants found that ORF overexpression of all four candidate genes significantly increased survival against T cell cytotoxicity in at least two additional cancer types (FIG. 5a-b and FIG. 7b-h). Overexpression of CD274 was not universally protective and did not confer resistance in cell lines with higher baseline expression, despite robust upregulation (FIG. 5b and FIG. 7h). The effects of MCL1 and B3GNT2 overexpression could be generalized to 6 and 7 of the additional cell lines respectively, demonstrating the broad applicability of these candidate genes to other cancer types and supporting the patient tumor analyses (FIG. 4a-b).

To test the relevance of candidate genes for immunotherapy in vivo, Applicants transduced A375 melanoma cells with dox-inducible candidate genes and subcutaneously engrafted these cells in immunocompromised NSG mice (FIG. 5c). At 2 days after subcutaneous tumor injection, Applicants induced overexpression of candidate genes, and at 7 days Applicants treated A375 xenografts with adoptive transfer of ESO T cells (FIG. 5c). In untreated control mice, Applicants did not observe significant differences in tumor growth or host survival between the candidate genes and GFP control (FIG. 7i-j). However, in mice treated with ESO T cells, overexpression of all four candidate genes significantly diminished the efficacy of adoptive cell transfer as measured by tumor growth and host survival (FIG. 5d-e). JUNB overexpression resulted in largely ineffective treatment, as JUNB-overexpressing xenografts displayed similar growth kinetics with and without ESO T cell treatment (FIG. 5d-e and FIG. 7i-j).

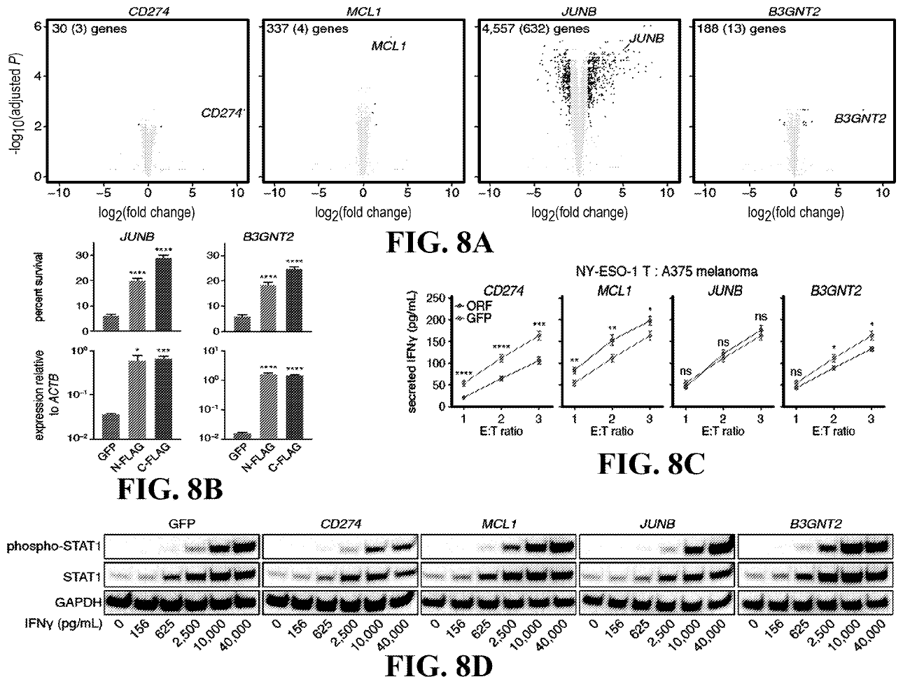

Applicants proceeded to investigate the mechanisms by which the candidate genes conferred resistance. As CD274 has been extensively studied (Reck et al., 2016), Applicants focused the mechanistic studies on the other three candidate genes. MCL1 is a BCL-2 family protein that inhibits apoptosis by regulating mitochondrial outer membrane permeabilization, and MCL1 overexpression is generally correlated with poor prognosis and resistance to most cancer therapeutics (Czabotar et al., 2014; Xiang et al., 2018). JUNB is a transcription factor that has been previously shown to downregulate an NKG2D ligand and mediate resistance against natural killer cells in mice (Nausch et al., 2006). B3GNT2 is a beta-1,3-N-acetylglucosaminyltransferase involved in poly-N-acetyllactosamine (poly-LacNAc) synthesis that has been suggested to glycosylate PD-1 in T cells to affect T cell activation (Sun et al., 2020). Moreover, B3GNT2 knockout mice have lower poly-LacNAc on B and T cells, resulting in hyperactivity (Togayachi et al., 2010). To begin to understand the pathways related to each candidate gene, Applicants performed RNA sequencing (RNA-seq) on A375 cells overexpressing each gene to characterize transcriptome changes. JUNB overexpression resulted in 632 differentially expressed genes with an absolute log fold change >1, compared to <15 genes for the other candidate genes, which is consistent with the role of JUNB in transcriptional regulation (FIG. 8a and Table 5). As the targets of JUNB and B3GNT2 are relatively unknown compared to MCL1, Applicants generated FLAG-tagged ORFs of both genes for immunoprecipitation assays (FIG. 8b). Chromatin immunoprecipitation sequencing (ChIP-seq) of JUNB and co-immunoprecipitation (co-IP) of B3GNT2 followed by mass spectrometry nominated 3,517 and 414 targets, respectively (Tables 6 and 7).

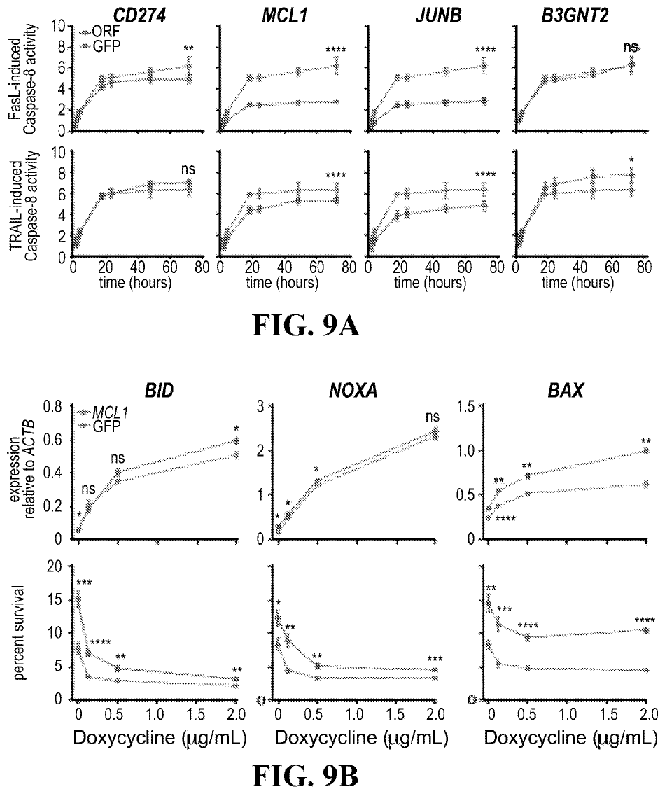

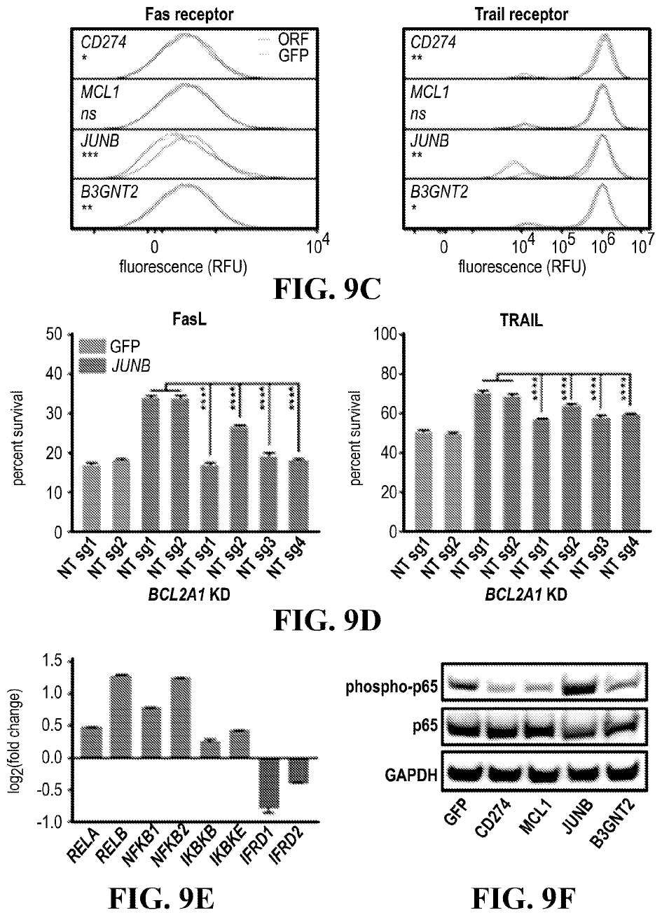

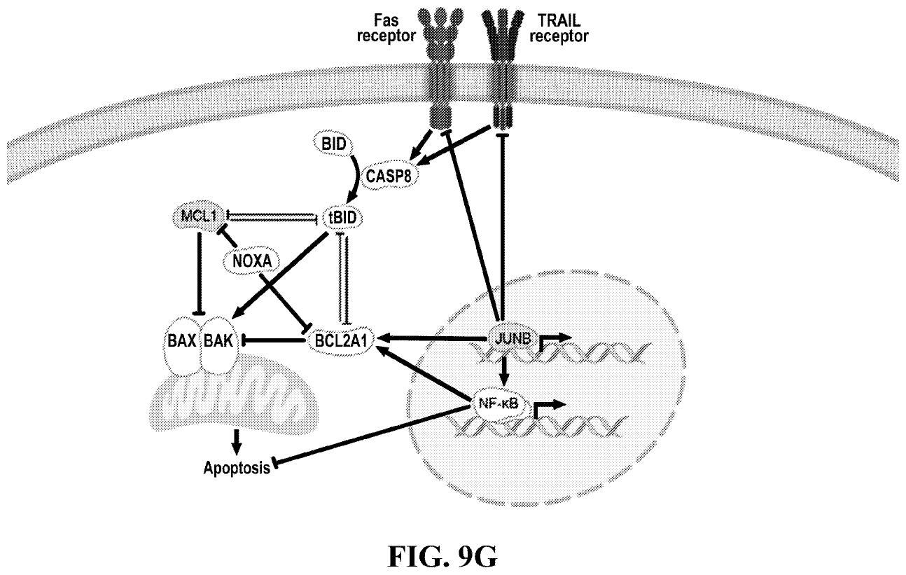

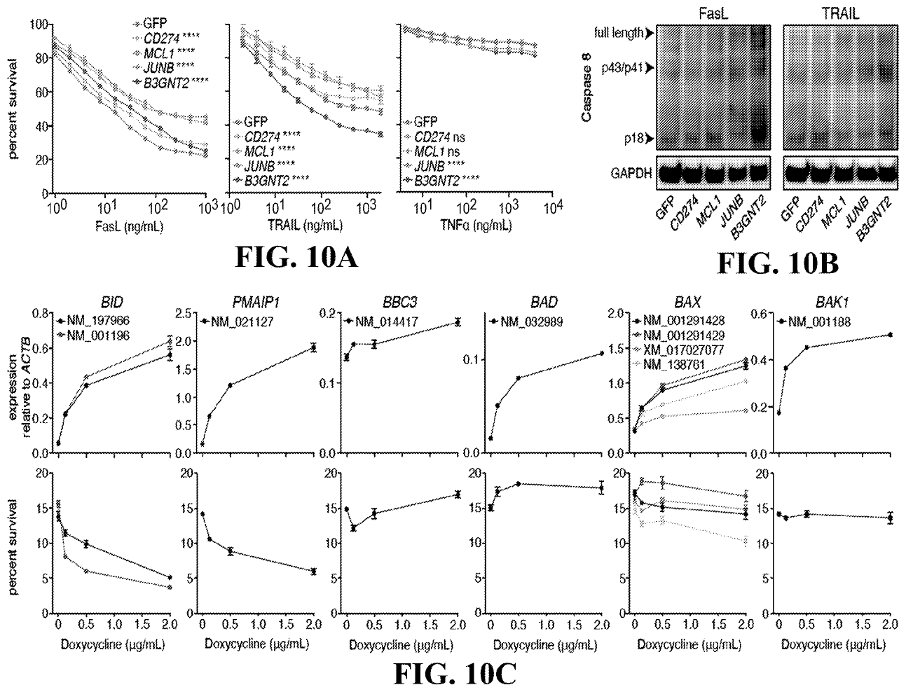

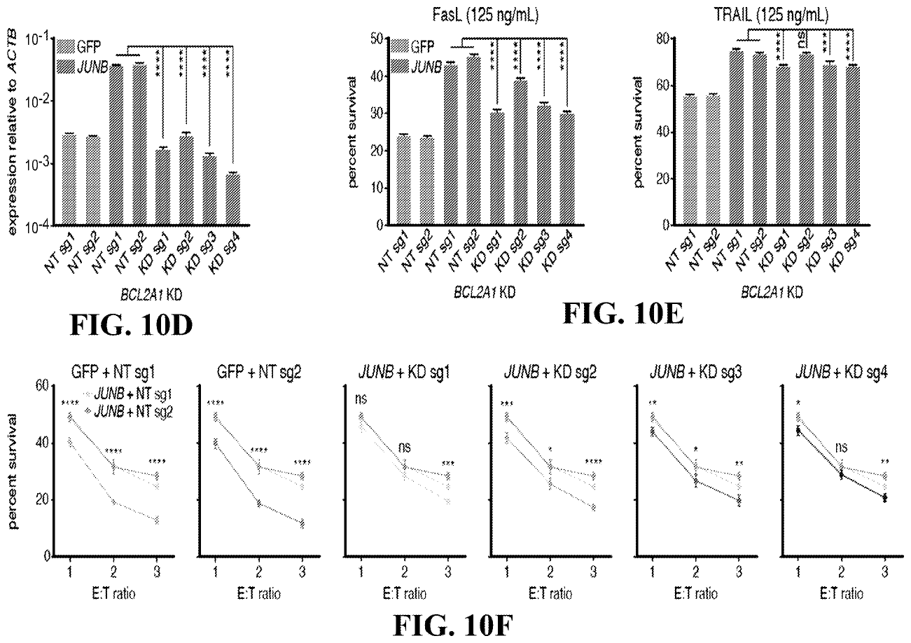

To narrow down the possible pathways to those that affect tumor immune evasion, Applicants assayed the effects of candidate gene overexpression on secretion and sensing of various cytokines involved in T cell cytotoxicity. Applicants quantified IFNγ released by T cells in the cytotoxicity assay using ELISA and found that upregulation of CD274 and B3GNT2 reduced IFNγ secretion by T cells (FIG. 8c). Overexpression of CD274 reduced the tumor response to IFNγ, as indicated by phosphorylation of STAT1, potentially resulting from a negative feedback mechanism (FIG. 8d) (Garcia-Diaz et al., 2017). Applicants challenged A375 cells overexpressing each of the candidate genes with cytokines that mediate cytotoxicity, FasL, TRAIL, or TNFα. Applicants found that MCL1 and JUNB overexpression significantly increased survival against FasL- and TRAIL-induced cell death (FIG. 9a and FIG. 10a-b).

Applicants examined components of the FasL and TRAIL signaling pathways that could contribute to MCL1- and JUNB-mediated resistance. For MCL1, the potential interaction partners involved in FasL and TRAIL resistance have been identified in previous studies (Czabotar et al., 2014). Applicants induced expression of these interaction partners in MCL1-overexpressing A375 cells and measured survival against T cell cytotoxicity. Induction of genes that more directly interact with MCL1, such as BID, PMAIP1 (NOXA), and BAX, could offset resistance conferred by MCL1 (FIG. 9b and FIG. 10c). For JUNB, the extensive list of target genes overlapping the ChIP- and RNA-seq datasets (Table 6) suggests there could be multiple components involved, necessitating a more systematic approach. Applicants first assayed cell surface expression of FasL and TRAIL receptors and found that JUNB overexpression significantly reduced expression of both receptors, FAS and TNFRSF10B (FIG. 9c). Applicants found that the JUNB target gene BCL2A1, an anti-apoptotic BCL-2 family protein that operates in parallel to MCL1 (Czabotar et al., 2014), was upregulated 45-fold and enriched in the set of 576 candidate genes from the CRISPRa screen, suggesting that BCL2A1 may also contribute to FasL and TRAIL resistance (Tables 2 and 6). CRISPR inhibition (CRISPRi) knockdown of BCL2A1 in JUNB-overexpressing cells significantly decreased survival against cytotoxicity induced by FasL, TRAIL, and T cells (FIG. 9d and FIG. 10d-f). In addition, JUNB alters expression of many different genes to activate the NF-κB pathway, including components of the NF-κB complex (RELA, RELB, NEKB1, and NFKB2), NF-κB activators (IKBKB and IKBKE), and NF-κB inhibitors (IFRD1) (Hoesel and Schmid, 2013; Liu et al., 2017; Tummers et al., 2015) (FIG. 9e). Perturbation of these genes by JI/NB results in activation of the NF-κB pathway, as indicated by phosphorylation of p65 (RELA) (FIG. 9f). Similar to JUNB, NF-κB activation upregulates BCL2A1, thus creating a feed forward loop for BCL2A1 upregulation (Grumont et al., 1999; Zong et al., 1999). The results show that MCL1 and JUNB counteract FasL- and TRAIL-induced cell death by inhibiting the mitochondrial apoptosis pathway, further supporting the importance of the death receptor signaling pathway in immunotherapy (Dufva et al., 2020; Singh et al., 2020) (FIG. 9g).

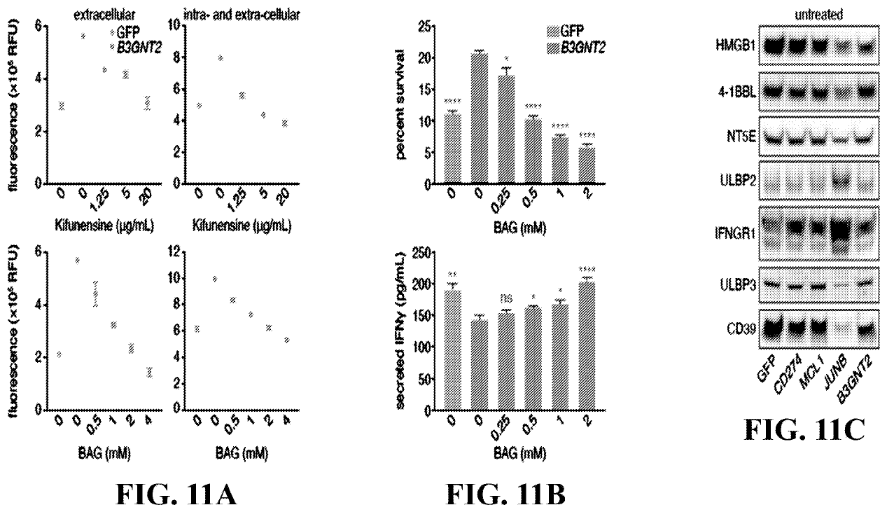

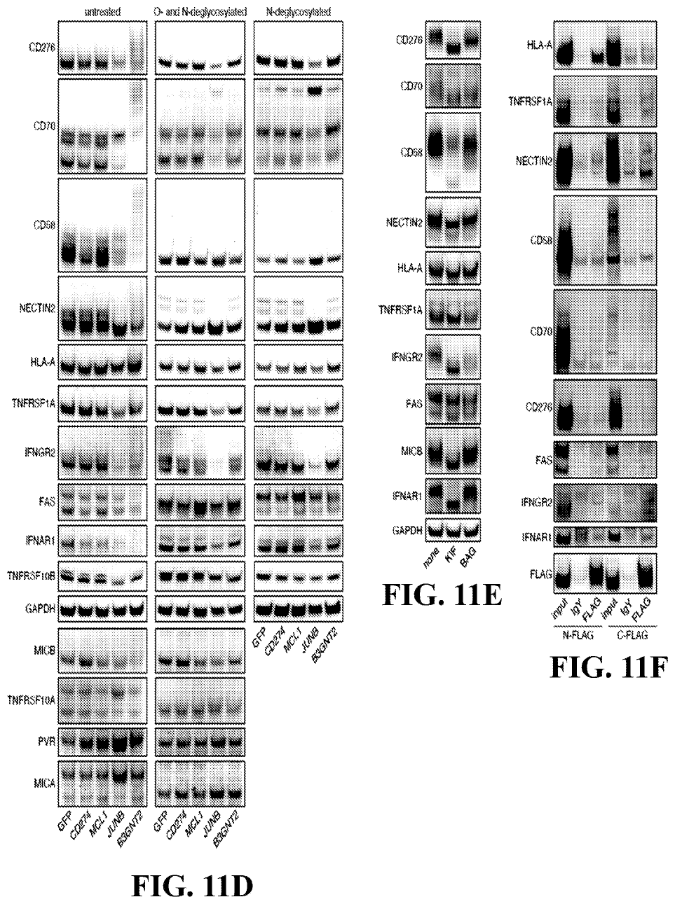

Next, Applicants turned to the resistance mechanism for B3GNT2. B3GNT2 overexpression in A375 cells increased intra- and extra-cellular poly-LacNAc as measured by tomato lectin staining (FIG. 11a). Pretreating cells with either N- or O-linked glycosylation inhibitors, kifunensine or benzyl-2-acetamido-2-deoxy-α-D-galactopyranoside (BAG) respectively, reduced poly-LacNAc added by B3GNT2 in a dosage-dependent manner (FIG. 11a). As T cells that were co-cultured with B3GNT2-overexpressing A375 cells secreted less IFNγ (FIG. 8c), Applicants tested whether glycosylation inhibition could restore T cell activation. Applicants found that pretreating B3GNT2-overexpressing A375 cells with both glycosylation inhibitors reversed the effects of B3GNT2 overexpression, resulting in increased T cell IFNγ secretion and reduced A375 survival, with kifunensine having a stronger effect (FIG. 12a and FIG. 11b). As interaction between T cell and tumor cell surface ligands and receptors triggers IFNγ secretion, Applicants assayed the ligands and receptors that were highly expressed in A375 for modifications by B3GNT2. Applicants found that many of these proteins showed higher and broader ranges of molecular weights on Western blots, potentially indicating increased presence of poly-LacNAc (FIG. 11c-d). Enzymatic deglycosylation of the proteins confirmed that the increased molecular weights represented glycosylation, not other post-translational modifications (FIG. 11d). Pretreating A375 cells overexpressing B3GNT2 with either kifunensine or BAG showed that the 10 ligands and receptors modified by B3GNT2 (CD276, CD70, CD58, NECTIN2, HLA-A, TNFRSF1A, IFNGR2, FAS, IFNAR1, MICB) are primarily N-glycosylated, aligning with the finding that kifunensine treatment had a stronger effect on T cell IFNγ secretion and tumor cell survival (FIG. 12b). Applicants found that these 10 ligands and receptors had increased presence of poly-LacNAc at baseline in SW1417 colorectal adenocarcinoma cells, which express higher levels of endogenous B3GNT2 than A375 cells (FIG. 11e). Co-IP of B3GNT2 verified that these ligands and receptors are B3GNT2 targets (FIG. 11f).

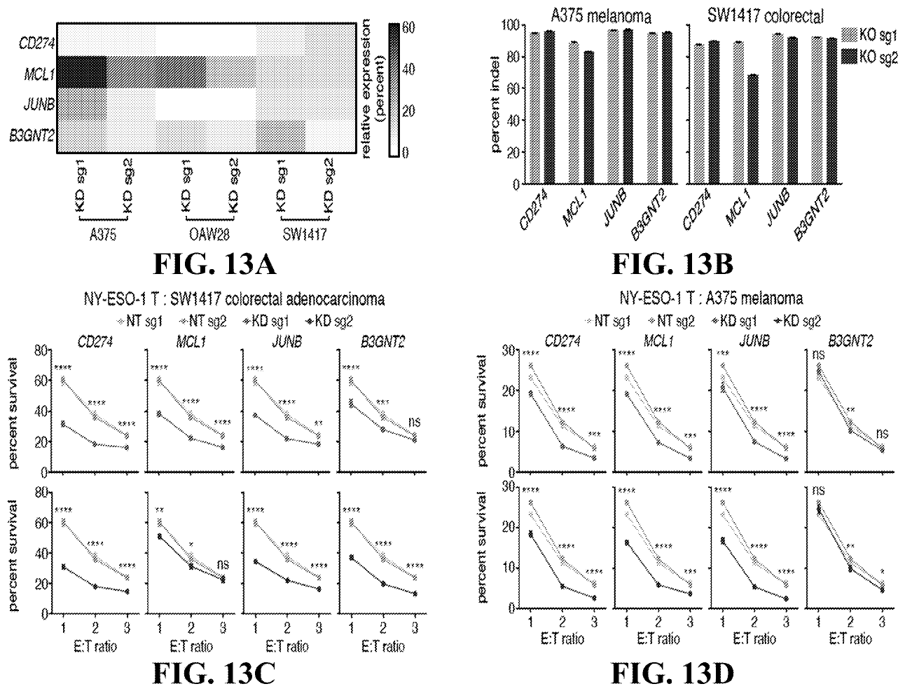

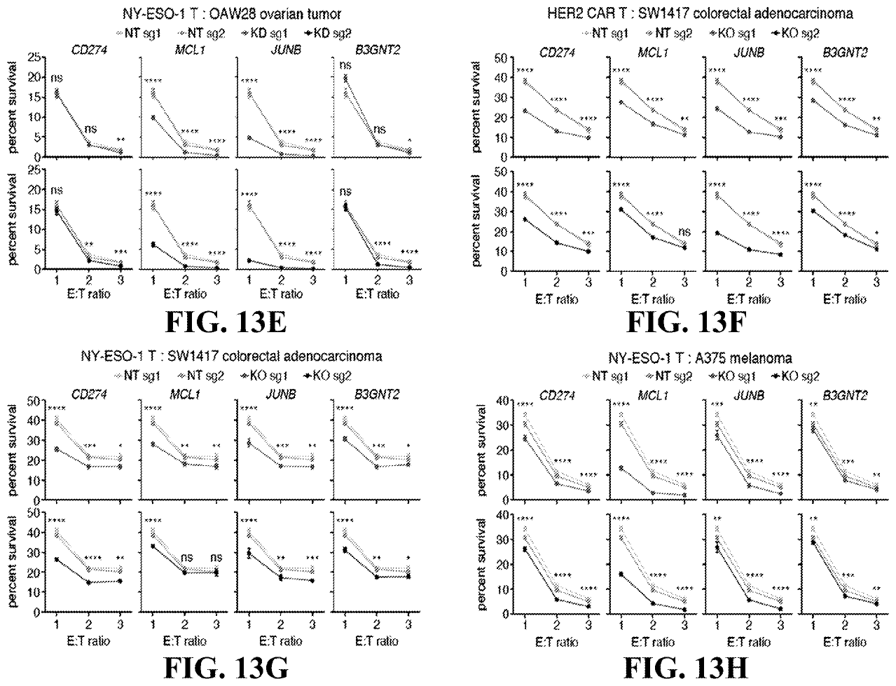

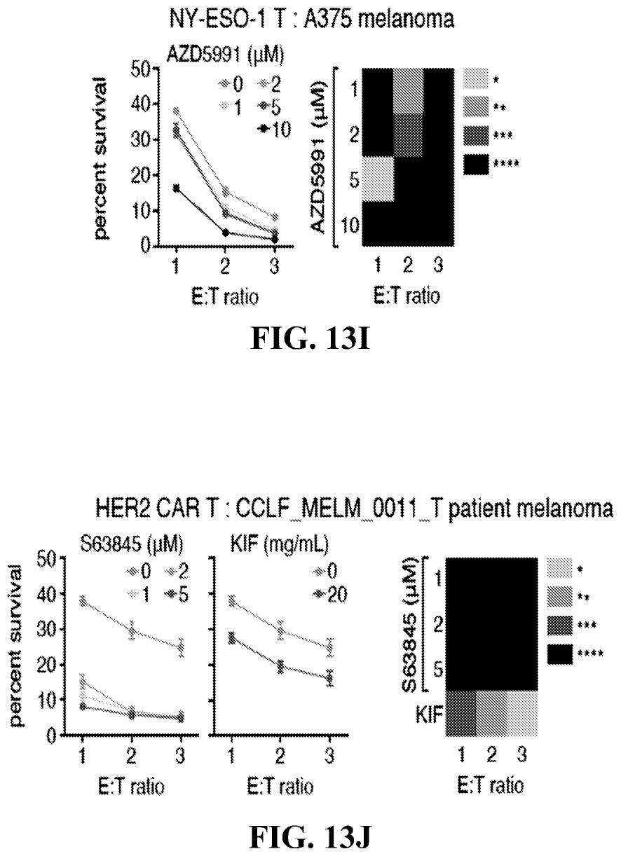

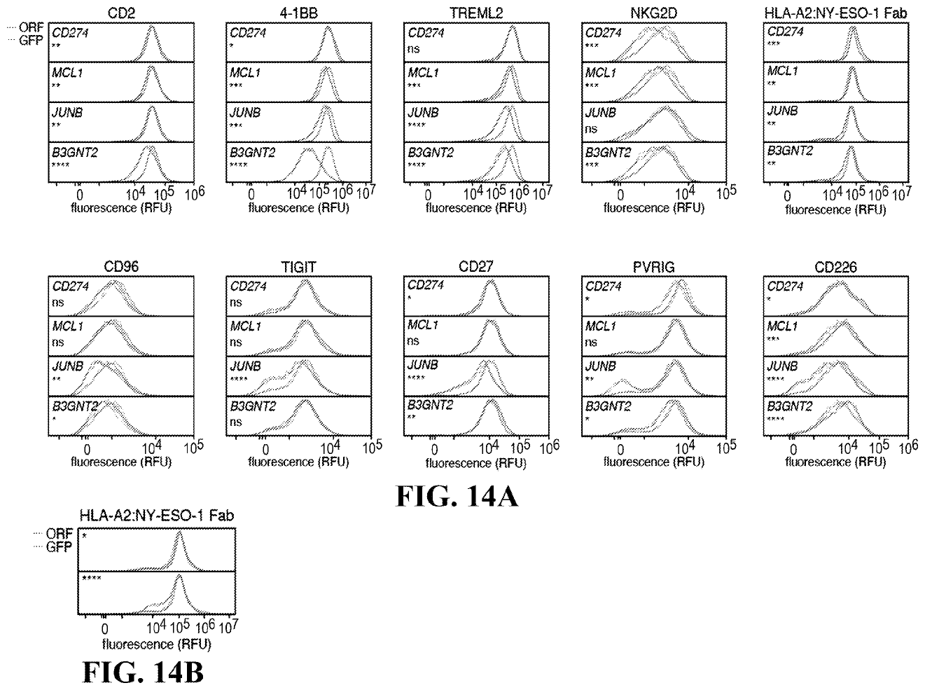

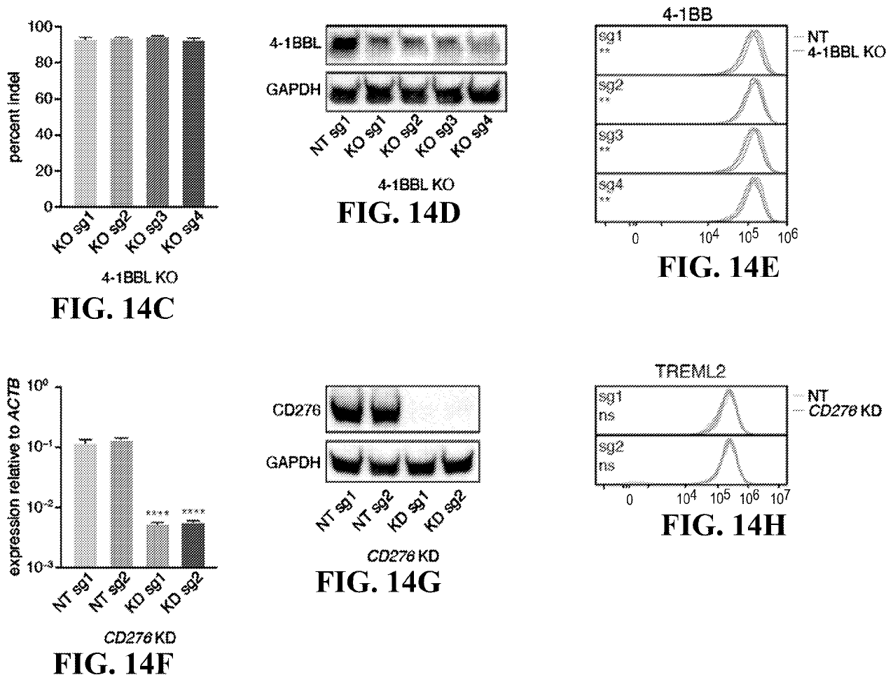

Applicants sought to determine whether increased poly-LacNAc on the B3GNT2 targets affected ligand-receptor interactions between tumor and T cells that facilitate T cell activation and subsequent cytotoxicity. By measuring binding of a panel of 10 recombinant T cell proteins to A375 cells overexpressing B3GNT2, Applicants found that binding of 5 T cell proteins [CD2, 4-1BB, TREML2 (TLT2), NKG2D, and an antibody specific for HLA-A2:NY-ESO-1] was significantly reduced (FIG. 13a). Treating B3GNT2-overexpressing A375 cells with kifunensine or BAG rescued the reduction in T cell protein binding (FIG. 12c). In the case of HLA-A2:NY-ESO-1 antibody binding, kifunensine further reduced binding, potentially because antigen presentation was disrupted (FIG. 13b). For T cell proteins CD2, NKG2D, and HLA-A2:NY-ESO-1 antibody, Applicants have demonstrated that their known tumor interaction partners, CD58, MICB, and HLA-A respectively, are B3GNT2 target proteins (Shaw et al., 1986; Steinle et al., 2001) (FIG. 12b-c and FIG. 14c). However, for 4-1BB, the mechanism was not immediately clear because its known interaction partner, 4-1BBL, was not modified by B3GNT2 (Alderson et al., 1994) (FIG. 14b). Applicants confirmed that that 4-1BB bound to 4-1BBL in the assay using CRISPR knockout (FIG. 13c-e). These results suggest two possibilities: 1) increased poly-LacNAc on other cell surface ligands and receptors targeted by B3GNT2 disrupts the 4-1BB/4-1BBL binding; 2) 4-1BB binds to other unknown ligands that are targeted by B3GNT2. For TREML2, though some studies have suggested that TREML2 interacts with the B3GNT2 target protein CD276 (Hashiguchi et al., 2008), CRISPR knockdown of CD276 did not affect binding to TREML2 (FIG. 13f-h). The finding aligns with a previous study showing that the interaction between TREML2 and CD276 does not occur in humans (Leitner et al., 2009). Taken together, Applicants have shown that B3GNT2 mediates resistance to T cell cytotoxicity by adding poly-LacNAc on numerous proteins to interfere with ligand-receptor interactions between tumor and T cells (FIG. 12d).

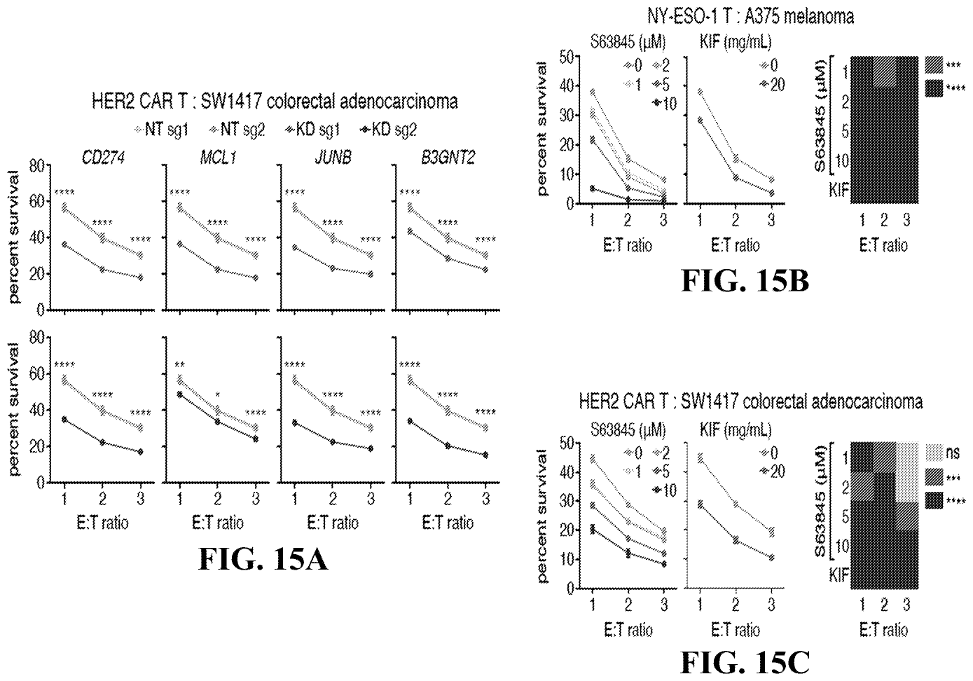

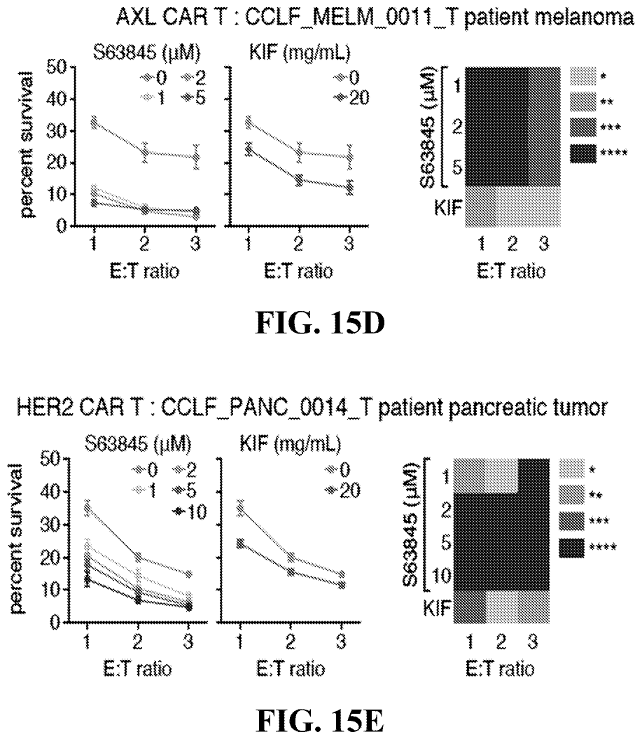

To test whether inhibition of candidate genes could produce the opposite effect and render tumors more susceptible to T cell cytotoxicity, Applicants designed CRISPR sgRNAs to knock down or knock out the four candidate genes and measured tumor survival against T cell killing (FIG. 13a-b). In SW1417 colorectal adenocarcinoma cells, which express relatively high levels of the candidate genes (FIG. 13a), knockdown of all four candidate genes significantly decreased cell survival when cells were co-cultured with HER2 CAR or ESO T cells (FIG. 15 and FIG. 13c). In A375 melanoma cells, knockdown of CD274, MCL1, and JUNB decreased cell survival, and in OAW28 ovarian cystadenocarcinoma cells, knockdown of MCL1 and JUNB decreased cell survival (FIG. 10d-e). Knockdown of B3GNT2 did not affect survival in A375 and OAW28 cell lines, potentially because B3GNT2 is expressed at relatively low levels in these cell lines (FIG. 13a). Applicants observed comparable results for candidate gene knockout in SW1417 and A375 cells (FIG. 13f-h). In addition to CRISPR perturbation, Applicants tested chemical inhibition of MCL1 and B3GNT2. Selective MCL1 inhibitors are already undergoing testing in clinical trials (Xiang et al., 2018) and the dosage of these inhibitors could be adjusted to preferentially target MCL1-dependent tumor cells. The resistance mechanism of B3GNT2 suggests that inhibition of extracellular poly-LacNAc could bolster immunotherapy. Applicants therefore inhibited MCL1 using selective small molecule inhibitors and B3GNT2 using kifunensine to generally inhibit N-linked glycosylation. Both chemical inhibition approaches reduced survival of A375 and SW1417 cells, as well as primary patient-derived melanoma and pancreatic adenocarcinoma models, against T cell cytotoxicity (FIG. 15b-e and FIG. 13i-j). The CRISPR and chemical inhibition results indicate that inhibition of candidate genes in tumor cells enhances T cell killing and can be combined with current immunotherapy strategies to improve efficacy.

More generally, the results suggest that inhibition of B3GNT2 and BCL-2 family proteins, MCL1 and BCL2A1, could enhance the efficacy of immunotherapy and improve patient response. The high cross-validation rate of MCL1 and B3GNT2 across different cancer cell types and their frequency in patient tumor types suggest that the resistance effects are relatively cell type independent. The distinct pathways of the candidate genes may have contributed to their respective differences in resistance to TCR and CAR T cell cytotoxicity. MCL1 and JUNB overexpression may result in higher resistance against CAR-expressing T cell cytotoxicity because CAR-mediated killing may rely more on the mitochondrial apoptosis pathway for cytotoxicity (Dufva et al., 2020; Singh et al., 2020). By contrast, B3GNT2 overexpression produces higher resistance against T cells expressing TCR than CAR because B3GNT2 confers resistance by disrupting interactions between tumor and T cells to reduce T cell activation. As the CAR design includes multiple intracellular co-stimulatory domains that promote T cell activation (Cho et al., 2018), CAR function is not as affected by these disruptions. Characterizing resistance mechanisms thus helps inform the choice between TCR- and CAR-based immunotherapy.

### CONCLUSIONS

Applicants have shown that genome-scale, gain-of-function genetic screens can discover genes involved in different biological processes that confer resistance to T cell cytotoxicity. Overexpression of candidate genes conferred resistance in diverse types of cancers. Mechanistic investigation revealed that MCL1 and JUNB overexpression mediate resistance to FasL- and TRAIL-induced cell death through the mitochondrial apoptosis pathway. JUNB downregulates FasL and TRAIL receptors, upregulates BCL2A1, and activates the NF-κB pathway. B3GNT2 promotes resistance through an orthogonal pathway by increasing poly-LacNAc on at least 10 tumor ligands and receptors to reduce T cell activation, highlighting the importance of poly-LacNAc in immuno-oncology. Furthermore, inhibition of these genes sensitized both tumor cell lines and primary patient-derived tumor models to T cell killing. This study complements results from previous loss-of-function screens and advances our understanding of the pathways that govern tumor immunotherapy.

TABLE 4
Pathway analysis of the 576 candidate genes. Pathways were ranked by FDR and pathways that overlapped
(>30% of genes) with another pathway that had lower FDR were removed to identify distinct pathways.

| | | | negative_{—} | | | |
|---|---|---|---|---|---|---|
| | | | log10_of_{—} | | | |
| | | adjusted_{—} | adjusted_{—} | term_{—} | intersection_{—} | |
| term_name | term_id | p_value | p_value | size | size | intersections |
| nucleic acid phosphodiester | GO:0090305 | 3.52E−07 | 6.453115912 | 151 | 12 | NOL9,HMGB2,CDKN2A,EXOSC9,RPA1, |
| bond hydrolysis | | | | | | POP7,OGG1,RPS27A,RFC2,XRN1, |
| negative regulation of Wnt | GO:0030178 | 2.93349E−05 | 4.532615968 | 194 | 11 | APC, PRICKLE1, NFATC1, AMER1, |
| signaling pathway | | | | | | TMEM170B, TLE3, TLE2, MDK, DKK2, |
| | | | | | | DAB2IP, PSMF1 |
| glycosaminoglycan metabolic | GO:0030203 | 5.21503E−05 | 4.282743425 | 132 | 9 | DSEL, LYVE1, B3GAT1, DCN, B3GNT2, |
| process | | | | | | SDC2, CHST13, SLC9A1, HMMR |
| negative regulation of cellular | GO:1903363 | 7.63E−05 | 4.117475546 | 75 | 7 | TRIM39, CDKN2A, UBE2J1, FURIN, |
| protein catabolic process | | | | | | DAB2IP, EFNA1, PSMF1 |
| cell-matrix adhesion | GO:0007160 | 0.000101361 | 3.994127445 | 183 | 10 | LYVE1, CD36, ADAMTS13, ADAMTS12, |
| | | | | | | SRC, CDKN2A, PTK2, SLC9A1, |
| | | | | | | SEMA3E, MAP4K4 |
| response to lipopolysaccharide | GO:0032496 | 0.000188756 | 3.724099352 | 198 | 10 | HMGB2, CD36, JUNB, CD14, IL1A, |
| | | | | | | TNIP2, TNIP3, CD274, IFNAR1, DAB2IP |
| cytokinesis | GO:0000910 | 0.000208771 | 3.680328845 | 160 | 9 | APC, CALM1, FLCN, EFHC1, SPTBN1, |
| | | | | | | CDC14A, FSD1, STAMBP, SEPTIN9 |
| carbohydrate catabolic process | GO:0016052 | 0.000293472 | 3.532433089 | 168 | 9 | PGM2L1, PRKAG1, CALM1, ENO2, |
| | | | | | | FUT6, ARNT, FUT10, GALK2, TREH |
| establishment or maintenance | GO:0007163 | 0.00033168 | 3.479280796 | 171 | 9 | CD3G, FSCN3, LMNA, PTK2, FLOT2, |
| of cell polarity | | | | | | CDC42BPB, AQP1, SLC9A1, ARFGEF1 |
| negative regulation of cellular | GO:0090288 | 0.000577538 | 3.238419431 | 144 | 8 | TGFBR2, DCN, ADAMTS12, NBL1, |
| response to growth factor | | | | | | VEPH1, RBPMS2, RPS27A, DAB2IP |
| stimulus | | | | | | |
| cellular response to hypoxia | GO:0071456 | 0.001132026 | 2.946143415 | 161 | 8 | LMNA, SUV39H1, ARNT, AQP1, TERT, |
| | | | | | | RPS27A, NFE2L2, PSMF1 |
| epithelial tube morphogenesis | GO:0060562 | 0.001218181 | 2.914288095 | 163 | 8 | TGFBR2, ADAMTS12, PRICKLE1, |
| | | | | | | BMP5, SETDB2, SEMA3E, HES1, MDK |
| extrinsic apoptotic signaling | GO:0097191 | 0.001357028 | 2.867411158 | 166 | 8 | BCL2A1, SRC, BMP5, MCL1, IL1A, |
| pathway | | | | | | TERT, GABARAP, DAB2IP |
| negative regulation of | GO:2001251 | 0.001685043 | 2.773388953 | 130 | 7 | CTBP1, APC, SRC, CDC20, BCOR, |
| chromosome organization | | | | | | TERF2IP, XRN1 |
| cell-cell adhesion via plasma- | GO:0098742 | 0.002463362 | 2.608471773 | 184 | 8 | TGFBR2, BSG, CLDN14, CLDN17, |
| membrane adhesion molecules | | | | | | MPZL2, CLDN8, ATP2C1, CDH18 |
| cold-induced thermogenesis | GO:0106106 | 0.002878731 | 2.540798977 | 144 | 7 | APC, CD36, FLCN, ACSL1, ESRRG, |
| | | | | | | TLE3, TSHR |
| calcium-mediated signaling | GO:0019722 | 0.00303933 | 2.517222177 | 191 | 8 | ATP2A2, CALM1, P2RX5, NFATC1, |
| | | | | | | SLC9A1, RCAN2, MCU, CCR4 |
| positive regulation of | GO:0050679 | 0.003822073 | 2.417700976 | 152 | 7 | HMGB2, BMP5, NME1, NME2, ARNT, |
| epithelial cell proliferation | | | | | | GATA2, MDK |
| cellular response to radiation | GO:0071478 | 0.003947548 | 2.403672563 | 153 | 7 | CALM1, INO80, CDKN1A, AQP1, |
| | | | | | | XRCC6, POLD3, GNGT1 |
| spindle organization | GO:0007051 | 0.004197215 | 2.377038798 | 155 | 7 | EFHC1, INO80, HAUS4, NEK6, |
| | | | | | | MAPRE2, FSD1, DCTN2 |
| protein complex | GO:0051259 | 0.004745874 | 2.323683798 | 159 | 7 | ITLN1, TIFA, SLC9A1, MPP2, ACACA, |
| oligomerization | | | | | | TDO2, MCU |
| mitochondrial gene expression | GO:0140053 | 0.00503924 | 2.297634934 | 161 | 7 | IARS2, RPUSD4, MRPL15, MRPL53, |
| | | | | | | WARS2, FASTKD2, CHCHD10 |
| regulation of signal | GO:1901796 | 0.005838568 | 2.233693662 | 167 | 7 | PRKAG1, MAPK11, PTTG1IP, RPA1, |
| transduction by p53 class | | | | | | RBBP8, RFC2, L3MBTL1 |
| mediator | | | | | | |
| platelet degranulation | GO:0002576 | 0.006328537 | 2.198696707 | 124 | 6 | CHID1, CD36, CALM1, ISLR, PCYOX1L, |
| | | | | | | APLP2 |
| positive regulation of I- | GO:0043123 | 0.006472052 | 2.188957982 | 171 | 7 | IL1A, TERF2IP, NEK6, TIFA, TNIP2, |
| kappaB kinase/NF-kappaB | | | | | | ATP2C1, PLEKHG5 |
| signaling | | | | | | |
| chromatin assembly | GO:0031497 | 0.007366838 | 2.132718855 | 176 | 7 | HMGB2, MBD3L2, MBD3L3, CDKN2A, |
| | | | | | | SETDB2, HIST1H2BB, SUV39H1 |
| translational initiation | GO:0006413 | 0.007741206 | 2.111191367 | 178 | 7 | YTHDF3, RPLP1, EIF4G3, RPL6, RPL15, |
| | | | | | | EIF2B3, RPS27A |
| negative regulation of binding | GO:0051100 | 0.008240855 | 2.084027708 | 133 | 6 | ATP2A2, ARHGAP28, PCSK9, CDKN1A, |
| | | | | | | BTAF1, PLN |
| activation of innate immune | GO:0002218 | 0.009265525 | 2.033129953 | 137 | 6 | PQBP1, SRC, KRAS, PRKACB, XRCC6, |
| response | | | | | | PSMF1 |
| negative regulation of | GO:1901343 | 0.01079629 | 1.966725447 | 192 | 7 | COL4A3, DCN, NFATC1, ANGPTL7, |
| vasculature development | | | | | | CARD10, SEMA3E, DAB2IP |
| potassium ion transmembrane | GO:0071805 | 0.011311093 | 1.946495423 | 194 | 7 | KCNH6, KCNF1, NEDD4L, KCNQ2, |
| transport | | | | | | SLC9A1, SLC12A6, ABCC8 |
| receptor signaling pathway via | GO:0007259 | 0.011348946 | 1.945044479 | 144 | 6 | SOCS2, GHR, STAMBP, IFNAR1, HES1, |
| JAK-STAT | | | | | | IFNA7 |
| transcription initiation from | GO:0006367 | 0.01157489 | 1.936483114 | 195 | 7 | TCF4, CCNC, ESRRG, CDKNIA, RXRG, |
| RNA polymerase II promoter | | | | | | RXRA, TEAD4 |
| keratinization | GO:0031424 | 0.01237825 | 1.907340752 | 198 | 7 | KLK14, KRTAP5-6, KRT31, KRT17, |
| | | | | | | KRT14, FURIN, KRTAP20-2 |
| protein acylation | GO:0043543 | 0.012702172 | 1.896122014 | 199 | 7 | CTBP1, CLIP3, FLCN, TERF2IP, HHATL, |
| | | | | | | CDYL, YEATS4 |
| cellular response to external | GO:0071496 | 0.012959226 | 1.887420931 | 200 | 7 | FLCN, PDK2, PCSK9, CDKN1A, AQP1, |
| stimulus | | | | | | SLC9A1, GABARAP |
| nucleotide biosynthetic | GO:0009165 | 0.012959226 | 1.887420931 | 200 | 7 | FLCN, PDK2, ACSL1, VPS9D1, ACACA, |
| process | | | | | | RNLS, DUT |
| protein import | GO:0017038 | 0.015216647 | 1.817681045 | 155 | 6 | PAM16, PRICKLE1, PTTG1IP, IPO7, |
| | | | | | | TNPO2, NXT2 |
| phosphatidylinositol metabolic | GO:0046488 | 0.017834966 | 1.748727716 | 162 | 6 | PIGO, PIGZ, SOCS2, TPTE2, BMX, DPM3 |
| process | | | | | | |
| response to steroid hormone | GO:0048545 | 0.023257859 | 1.633430274 | 174 | 6 | SRC, CATSPERG, UBA5, GRIP1, AQP1, |
| | | | | | | STRN3 |
| regulation of protein | GO:0032271 | 0.028341083 | 1.547583559 | 183 | 6 | CLIP3, ARHGAP28, ACTR2, IQGAP2, |
| polymerization | | | | | | ARFGEF1, PLEKHG2 |
| antigen processing and | GO:0048002 | 0.031622848 | 1.499999013 | 189 | 6 | CD36, AP1B1, TAPBPL, DCTN2, PSMF1, |
| presentation of peptide antigen | | | | | | PDIA3 |

TABLE 5
RNA-seq analysis of differentially expressed genes for candidate ORF overexpression. Genes with > 1-fold change shown.
JUNB Genes

| Isoform | Fold | Pval | Padj | Isoform | Fold | Pval | Padj | Isoform | Fold | Pval | Padj |
|---|---|---|---|---|---|---|---|---|---|---|---|
| LCE1F | 279.05 | 1.63E−10 | 9.33E−07 | NOMO2 | 1.46 | 3.75E−05 | 2.80E−04 | BTBD7 | 1.25 | 5.63E−04 | 1.85E−03 |
| LRRC38 | 161.85 | 2.04E−10 | 9.33E−07 | TRIM58 | 1.74 | 3.77E−05 | 2.81E−04 | DAAM2 | 1.19 | 5.63E−04 | 1.85E−03 |
| LRFN5 | 25.73 | 1.58E−09 | 3.61E−06 | COPA | 1.17 | 3.82E−05 | 2.84E−04 | ANKRD10 | 1.52 | 5.63E−04 | 1.85E−03 |
| CD24 | 29.87 | 2.02E−09 | 3.69E−06 | ZNF385A | 1.53 | 3.84E−05 | 2.85E−04 | SLC18B1 | 1.64 | 5.64E−04 | 1.85E−03 |
| KRT6B | 9.32 | 3.71E−09 | 4.23E−06 | GNB2 | 1.15 | 3.98E−05 | 2.91E−04 | ADCY7 | 1.35 | 5.65E−04 | 1.85E−03 |
| TGFBI | 7.91 | 3.47E−09 | 4.23E−06 | SYT11 | 2.01 | 3.98E−05 | 2.91E−04 | ZDHHC9 | 1.35 | 5.65E−04 | 1.85E−03 |
| CYB5R2 | 5.20 | 8.57E−09 | 7.11E−06 | PCSK1N | 2.14 | 4.00E−05 | 2.91E−04 | DAB2IP | 1.27 | 5.72E−04 | 1.87E−03 |
| PPIF | 1.79 | 8.05E−09 | 7.11E−06 | ARHGEF12 | 1.24 | 4.03E−05 | 2.92E−04 | PPP2R1B | 1.41 | 5.77E−04 | 1.88E−03 |
| HAS2 | 47.21 | 1.47E−08 | 8.56E−06 | PLS1 | 2.23 | 4.08E−05 | 2.95E−04 | LRFN4 | 1.43 | 5.78E−04 | 1.89E−03 |
| ACAN | 46.27 | 1.70E−08 | 8.62E−06 | MICA | 1.20 | 4.10E−05 | 2.96E−04 | CSNK1A1 | 1.16 | 5.78E−04 | 1.89E−03 |
| JUNB | 33.09 | 1.89E−08 | 8.62E−06 | RASD2 | 6.50 | 4.10E−05 | 2.96E−04 | KCTD5 | 1.17 | 5.83E−04 | 1.90E−03 |
| COL22A1 | 72.60 | 2.62E−08 | 9.89E−06 | OSBPL3 | 1.39 | 4.12E−05 | 2.97E−04 | STX6 | 1.42 | 5.83E−04 | 1.90E−03 |
| FERMT1 | 38.24 | 2.46E−08 | 9.89E−06 | PTPRU | 1.64 | 4.12E−05 | 2.97E−04 | IFFO2 | 1.38 | 5.87E−04 | 1.91E−03 |
| MSN | 1.66 | 2.71E−08 | 9.89E−06 | SQRDL | 3.91 | 4.16E−05 | 2.99E−04 | BACH1 | 1.26 | 5.88E−04 | 1.91E−03 |
| ARNTL2 | 6.30 | 3.14E−08 | 1.05E−05 | GPR158 | 3.44 | 4.20E−05 | 3.00E−04 | OBFC1 | 1.36 | 5.91E−04 | 1.92E−03 |
| HLA-B | 1.95 | 3.45E−08 | 1.05E−05 | RASSF3 | 1.30 | 4.20E−05 | 3.00E−04 | ARHGAP1 | 1.33 | 5.93E−04 | 1.92E−03 |
| LAMB3 | 7.45 | 3.19E−08 | 1.05E−05 | SLC38A2 | 1.41 | 4.22E−05 | 3.01E−04 | RAPGEF1 | 1.27 | 5.94E−04 | 1.92E−03 |
| PDPN | 110.29 | 3.36E−08 | 1.05E−05 | RIPK4 | 1.85 | 4.23E−05 | 3.01E−04 | KIF16B | 1.35 | 5.97E−04 | 1.93E−03 |
| MAST4 | 13.70 | 3.67E−08 | 1.08E−05 | SALL1 | 1.37 | 4.22E−05 | 3.01E−04 | PAFAH2 | 1.08 | 5.97E−04 | 1.93E−03 |
| S100A16 | 2.51 | 3.80E−08 | 1.08E−05 | HBEGF | 2.76 | 4.28E−05 | 3.04E−04 | C16orf58 | 1.09 | 6.00E−04 | 1.93E−03 |
| NREP | 3.21 | 4.18E−08 | 1.16E−05 | ZMAT3 | 1.38 | 4.29E−05 | 3.04E−04 | OSTF1 | 1.49 | 6.04E−04 | 1.95E−03 |
| ALK | 6.65 | 6.04E−08 | 1.25E−05 | SULT1B1 | 15.01 | 4.31E−05 | 3.04E−04 | DNAJC10 | 1.27 | 6.10E−04 | 1.97E−03 |
| ANPEP | 3.38 | 6.64E−08 | 1.25E−05 | FAM168A | 1.46 | 4.36E−05 | 3.07E−04 | RPS3 | 1.08 | 6.15E−04 | 1.98E−03 |
| BMP6 | 11.58 | 6.45E−08 | 1.25E−05 | ASAH1 | 1.57 | 4.37E−05 | 3.08E−04 | HAPLN3 | 1.58 | 6.16E−04 | 1.98E−03 |
| CD59 | 2.39 | 5.19E−08 | 1.25E−05 | TRIM21 | 2.00 | 4.37E−05 | 3.08E−04 | CHMP5 | 1.27 | 6.19E−04 | 1.99E−03 |
| ESM1 | 22.77 | 6.42E−08 | 1.25E−05 | MAPKBP1 | 1.40 | 4.43E−05 | 3.10E−04 | ERRFI1 | 1.13 | 6.36E−04 | 2.03E−03 |
| FURIN | 1.86 | 6.48E−08 | 1.25E−05 | CANX | 1.08 | 4.65E−05 | 3.22E−04 | KLHL36 | 1.25 | 6.37E−04 | 2.03E−03 |
| GAP43 | 5.92 | 6.04E−08 | 1.25E−05 | ENDOD1 | 2.38 | 4.66E−05 | 3.23E−04 | DALRD3 | 1.15 | 6.38E−04 | 2.03E−03 |
| HLA-A | 2.24 | 7.14E−08 | 1.25E−05 | RPS20 | 1.08 | 4.70E−05 | 3.25E−04 | KLHL5 | 1.48 | 6.38E−04 | 2.03E−03 |
| IGFBP3 | 7.10 | 6.57E−08 | 1.25E−05 | SSR3 | 1.18 | 4.73E−05 | 3.26E−04 | WASF2 | 1.14 | 6.40E−04 | 2.04E−03 |
| INHBA | 7.51 | 6.79E−08 | 1.25E−05 | TCAF1 | 1.25 | 4.73E−05 | 3.26E−04 | ERCC5 | 1.38 | 6.45E−04 | 2.05E−03 |
| JAG1 | 3.94 | 5.88E−08 | 1.25E−05 | ZBTB38 | 1.33 | 4.73E−05 | 3.26E−04 | SLK | 1.24 | 6.45E−04 | 2.05E−03 |
| KIAA0040 | 9.87 | 5.99E−08 | 1.25E−05 | SAT1 | 2.51 | 4.76E−05 | 3.26E−04 | MNT | 1.25 | 6.46E−04 | 2.05E−03 |
| KLHL21 | 2.48 | 7.01E−08 | 1.25E−05 | SERPIND1 | 3.83 | 4.76E−05 | 3.27E−04 | ZPR1 | 1.20 | 6.54E−04 | 2.07E−03 |
| STRA6 | 2.68 | 7.38E−08 | 1.25E−05 | FXYD5 | 1.32 | 4.78E−05 | 3.27E−04 | TAP1 | 1.32 | 6.58E−04 | 2.08E−03 |
| TAGLN2 | 1.82 | 5.22E−08 | 1.25E−05 | XIAP | 1.25 | 4.80E−05 | 3.27E−04 | MGST1 | 1.24 | 6.59E−04 | 2.08E−03 |
| VCAN | 14.73 | 5.96E−08 | 1.25E−05 | RAB22A | 1.43 | 4.81E−05 | 3.28E−04 | PBXIP1 | 1.31 | 6.63E−04 | 2.09E−03 |
| ZNF611 | 3.71 | 6.53E−08 | 1.25E−05 | AKAP13 | 1.19 | 4.87E−05 | 3.31E−04 | FBXW4 | 1.27 | 6.67E−04 | 2.10E−03 |
| KCNG1 | 3.26 | 8.83E−08 | 1.37E−05 | LINC00623 | 1.81 | 4.88E−05 | 3.31E−04 | YKT6 | 1.17 | 6.69E−04 | 2.11E−03 |
| FLRT2 | 10.17 | 9.30E−08 | 1.41E−05 | RGS19 | 1.73 | 4.92E−05 | 3.32E−04 | C16orf45 | 1.23 | 6.75E−04 | 2.12E−03 |
| ATP6V0A4 | 9.02 | 9.76E−08 | 1.41E−05 | TLDC1 | 2.10 | 4.91E−05 | 3.32E−04 | CREG1 | 1.15 | 6.79E−04 | 2.13E−03 |
| RHOC | 1.90 | 1.19E−07 | 1.62E−05 | TRAPPC1 | 1.30 | 4.91E−05 | 3.32E−04 | INTS7 | 1.15 | 6.79E−04 | 2.13E−03 |
| S100A10 | 1.83 | 1.17E−07 | 1.62E−05 | NAGK | 1.61 | 5.00E−05 | 3.36E−04 | SLC33A1 | 1.52 | 6.80E−04 | 2.13E−03 |
| CDYL2 | 3.29 | 1.24E−07 | 1.67E−05 | SLFN5 | 2.56 | 5.03E−05 | 3.38E−04 | ZC3HAV1 | 1.24 | 6.90E−04 | 2.15E−03 |
| BTBD11 | 16.62 | 1.29E−07 | 1.69E−05 | PDE4A | 1.47 | 5.05E−05 | 3.38E−04 | FKBP9 | 1.16 | 6.93E−04 | 2.16E−03 |
| LEMD1 | 6.37 | 1.37E−07 | 1.69E−05 | QSOX1 | 1.46 | 5.09E−05 | 3.40E−04 | ANXA4 | 1.34 | 6.98E−04 | 2.17E−03 |
| PAPSS2 | 3.19 | 1.37E−07 | 1.69E−05 | DNAJC5 | 1.13 | 5.20E−05 | 3.46E−04 | CYLD | 1.38 | 6.99E−04 | 2.17E−03 |
| PI3 | 45.17 | 1.36E−07 | 1.69E−05 | EEF2 | 1.21 | 5.30E−05 | 3.50E−04 | SMIM3 | 1.94 | 6.99E−04 | 2.17E−03 |
| STEAP3 | 2.33 | 1.34E−07 | 1.69E−05 | MAP4K4 | 1.13 | 5.30E−05 | 3.50E−04 | TMEM208 | 1.17 | 7.01E−04 | 2.18E−03 |
| TFPI2 | 4.76 | 1.30E−07 | 1.69E−05 | ATXN10 | 1.23 | 5.36E−05 | 3.53E−04 | LOC102723724 | 1.24 | 7.02E−04 | 2.18E−03 |
| ACOT7 | 1.71 | 1.59E−07 | 1.73E−05 | KYNU | 1.35 | 5.37E−05 | 3.54E−04 | RALGPS2 | 1.49 | 7.05E−04 | 2.19E−03 |
| CDKN1A | 1.95 | 1.48E−07 | 1.73E−05 | CDIPT | 1.32 | 5.39E−05 | 3.55E−04 | FLT3LG | 1.66 | 7.06E−04 | 2.19E−03 |
| COL6A3 | 4.73 | 1.53E−07 | 1.73E−05 | RNF182 | 2.34 | 5.39E−05 | 3.55E−04 | LIX1L | 1.21 | 7.07E−04 | 2.19E−03 |
| IL24 | 4.20 | 1.59E−07 | 1.73E−05 | MED13 | 1.46 | 5.40E−05 | 3.55E−04 | PLPPR2 | 1.28 | 7.07E−04 | 2.19E−03 |
| OPTN | 2.90 | 1.49E−07 | 1.73E−05 | SETD7 | 1.34 | 5.52E−05 | 3.61E−04 | COX6B1 | 1.14 | 7.09E−04 | 2.19E−03 |
| CD55 | 3.45 | 1.66E−07 | 1.75E−05 | MGST3 | 2.15 | 5.53E−05 | 3.61E−04 | TMEM245 | 1.17 | 7.10E−04 | 2.19E−03 |
| RPS4X | 1.25 | 1.65E−07 | 1.75E−05 | RRM2B | 1.38 | 5.61E−05 | 3.65E−04 | PYGL | 1.13 | 7.14E−04 | 2.20E−03 |
| C1orf21 | 3.83 | 1.75E−07 | 1.82E−05 | ARPC5L | 1.17 | 5.65E−05 | 3.67E−04 | TMEM259 | 1.13 | 7.14E−04 | 2.20E−03 |
| SERPINB2 | 41.26 | 1.78E−07 | 1.83E−05 | SQSTM1 | 1.27 | 5.69E−05 | 3.69E−04 | TMEM102 | 1.53 | 7.15E−04 | 2.20E−03 |
| DUSP5 | 3.01 | 1.82E−07 | 1.84E−05 | TNIP1 | 1.63 | 5.70E−05 | 3.69E−04 | IKBKE | 1.31 | 7.17E−04 | 2.21E−03 |
| NRP1 | 3.02 | 1.90E−07 | 1.88E−05 | SNX8 | 1.28 | 5.71E−05 | 3.70E−04 | EHBP1L1 | 1.13 | 7.18E−04 | 2.21E−03 |
| TTPAL | 1.49 | 1.96E−07 | 1.92E−05 | DNER | 8.39 | 5.72E−05 | 3.70E−04 | PRDX5 | 1.27 | 7.20E−04 | 2.21E−03 |
| CHMP4C | 4.70 | 2.01E−07 | 1.95E−05 | PPP1R14B | 1.42 | 5.78E−05 | 3.73E−04 | ISG20 | 1.11 | 7.21E−04 | 2.21E−03 |
| MMP14 | 2.79 | 2.12E−07 | 2.02E−05 | FRMD3 | 2.09 | 5.84E−05 | 3.76E−04 | GRIPAP1 | 1.27 | 7.25E−04 | 2.22E−03 |
| B3GNT2 | 2.68 | 2.22E−07 | 2.06E−05 | DNM3 | 2.38 | 5.86E−05 | 3.76E−04 | ZBED1 | 1.36 | 7.25E−04 | 2.22E−03 |
| PDZRN3 | 9.28 | 2.20E−07 | 2.06E−05 | LRRC8A | 1.24 | 5.87E−05 | 3.76E−04 | ZFAND1 | 1.33 | 7.25E−04 | 2.22E−03 |
| MCL1 | 1.53 | 2.30E−07 | 2.12E−05 | NDUFB8 | 1.35 | 5.87E−05 | 3.76E−04 | MICAL1 | 1.41 | 7.27E−04 | 2.23E−03 |
| TMCC2 | 6.66 | 2.33E−07 | 2.12E−05 | ARPC5 | 1.24 | 5.94E−05 | 3.80E−04 | ATRAID | 1.08 | 7.28E−04 | 2.23E−03 |
| AMOTL1 | 2.17 | 2.42E−07 | 2.12E−05 | KLF5 | 1.65 | 5.96E−05 | 3.81E−04 | NSMAF | 1.18 | 7.28E−04 | 2.23E−03 |
| KDM6B | 2.19 | 2.44E−07 | 2.12E−05 | FLOT1 | 1.46 | 5.97E−05 | 3.81E−04 | CBFB | 1.21 | 7.33E−04 | 2.24E−03 |
| FBXO27 | 2.95 | 2.52E−07 | 2.13E−05 | ANKRD13A | 1.35 | 6.04E−05 | 3.84E−04 | NR3C1 | 1.32 | 7.35E−04 | 2.24E−03 |
| TAGLN3 | 7.21 | 2.52E−07 | 2.13E−05 | EAF1 | 1.21 | 6.06E−05 | 3.85E−04 | UFC1 | 1.30 | 7.37E−04 | 2.25E−03 |
| BCL2A1 | 32.36 | 2.56E−07 | 2.14E−05 | ATP6V1G1 | 1.24 | 6.07E−05 | 3.86E−04 | NBPF14 | 1.24 | 7.40E−04 | 2.25E−03 |
| PMEPA1 | 2.22 | 2.61E−07 | 2.15E−05 | LINC01057 | 1.62 | 6.08E−05 | 3.86E−04 | LINC00504 | 1.15 | 7.42E−04 | 2.26E−03 |
| ITGA2 | 7.39 | 2.71E−07 | 2.17E−05 | DLX2 | 2.24 | 6.13E−05 | 3.88E−04 | FAM114A1 | 1.26 | 7.46E−04 | 2.27E−03 |
| PLEKHG5 | 3.50 | 2.66E−07 | 2.17E−05 | VEGFB | 1.28 | 6.17E−05 | 3.89E−04 | DGCR2 | 1.13 | 7.47E−04 | 2.27E−03 |
| SCEL | 26.37 | 2.71E−07 | 2.17E−05 | EFL1 | 1.57 | 6.18E−05 | 3.90E−04 | RPS28 | 1.07 | 7.50E−04 | 2.28E−03 |
| TMEM158 | 15.72 | 2.78E−07 | 2.17E−05 | KIF13A | 1.88 | 6.39E−05 | 3.99E−04 | SLC1A1 | 1.60 | 7.56E−04 | 2.29E−03 |
| CLMP | 7.17 | 3.15E−07 | 2.21E−05 | COL9A3 | 1.31 | 6.44E−05 | 4.01E−04 | FRMD4A | 1.20 | 7.57E−04 | 2.29E−03 |
| CXCL14 | 33.95 | 3.13E−07 | 2.21E−05 | RNF213 | 1.23 | 6.45E−05 | 4.02E−04 | PARP12 | 1.73 | 7.68E−04 | 2.32E−03 |
| LAMC1 | 1.40 | 3.10E−07 | 2.21E−05 | APLP1 | 2.35 | 6.47E−05 | 4.03E−04 | CEP170 | 1.15 | 7.70E−04 | 2.32E−03 |
| MMP1 | 16.55 | 3.04E−07 | 2.21E−05 | EDNRB | 2.41 | 6.54E−05 | 4.06E−04 | STX2 | 1.34 | 7.72E−04 | 2.32E−03 |
| MYH9 | 1.87 | 2.98E−07 | 2.21E−05 | FHOD1 | 1.17 | 6.55E−05 | 4.07E−04 | MAGED2 | 1.33 | 7.75E−04 | 2.33E−03 |
| MYO1D | 3.73 | 2.93E−07 | 2.21E−05 | CACNB3 | 1.46 | 6.57E−05 | 4.07E−04 | CERCAM | 1.27 | 7.78E−04 | 2.34E−03 |
| NRCAM | 4.15 | 2.99E−07 | 2.21E−05 | GLRX | 1.62 | 6.59E−05 | 4.07E−04 | MIR100HG | 1.19 | 7.80E−04 | 2.34E−03 |
| PHLDB3 | 2.49 | 3.01E−07 | 2.21E−05 | FAM127A | 1.48 | 6.60E−05 | 4.07E−04 | CLDND1 | 1.25 | 7.81E−04 | 2.34E−03 |
| RTL1 | 7.42 | 3.08E−07 | 2.21E−05 | NIPA1 | 1.22 | 6.67E−05 | 4.11E−04 | TM9SF3 | 1.25 | 7.83E−04 | 2.35E−03 |
| S100A6 | 1.71 | 2.87E−07 | 2.21E−05 | MROH1 | 1.76 | 6.77E−05 | 4.17E−04 | PNPLA8 | 1.22 | 7.90E−04 | 2.37E−03 |
| MMP10 | 43.42 | 3.22E−07 | 2.25E−05 | MFHAS1 | 1.55 | 6.83E−05 | 4.18E−04 | LGMN | 1.67 | 7.97E−04 | 2.38E−03 |
| NTSR1 | 4.74 | 3.48E−07 | 2.29E−05 | SNX29 | 1.35 | 6.83E−05 | 4.18E−04 | IL10RB | 1.21 | 7.99E−04 | 2.38E−03 |
| TRPV2 | 2.00 | 3.46E−07 | 2.29E−05 | DBN1 | 1.42 | 6.84E−05 | 4.19E−04 | PLK3 | 1.32 | 8.01E−04 | 2.39E−03 |
| CTSS | 15.14 | 3.65E−07 | 2.32E−05 | TPBG | 1.42 | 6.84E−05 | 4.19E−04 | DNAJC4 | 1.27 | 8.07E−04 | 2.40E−03 |
| S100A1 | 2.18 | 3.64E−07 | 2.32E−05 | RAI14 | 1.60 | 6.89E−05 | 4.21E−04 | CHIC2 | 1.26 | 8.14E−04 | 2.41E−03 |
| SDC1 | 3.19 | 3.67E−07 | 2.32E−05 | RAP2B | 2.06 | 6.89E−05 | 4.21E−04 | TIPARP | 1.25 | 8.23E−04 | 2.44E−03 |
| SETBP1 | 4.44 | 3.68E−07 | 2.32E−05 | IFI30 | 1.43 | 7.03E−05 | 4.26E−04 | IL1RAPL1 | 1.61 | 8.27E−04 | 2.44E−03 |
| TMEM2 | 3.18 | 3.68E−07 | 2.32E−05 | MORF4L2 | 1.13 | 7.04E−05 | 4.26E−04 | ZBTB4 | 1.25 | 8.29E−04 | 2.45E−03 |
| NR4A1 | 2.01 | 3.78E−07 | 2.35E−05 | MYSM1 | 1.62 | 7.05E−05 | 4.26E−04 | ACO1 | 1.12 | 8.31E−04 | 2.45E−03 |
| WNT5A | 2.66 | 3.75E−07 | 2.35E−05 | RCN1 | 1.33 | 7.05E−05 | 4.26E−04 | TMEM258 | 1.24 | 8.33E−04 | 2.46E−03 |
| ERGIC1 | 2.14 | 3.91E−07 | 2.36E−05 | SHROOM4 | 1.70 | 7.04E−05 | 4.26E−04 | NOL9 | 1.27 | 8.35E−04 | 2.46E−03 |
| KIAA1549L | 2.41 | 3.87E−07 | 2.36E−05 | SVIL | 1.50 | 7.05E−05 | 4.26E−04 | TMEM127 | 1.28 | 8.51E−04 | 2.50E−03 |
| SLC1A6 | 50.01 | 3.92E−07 | 2.36E−05 | CD96 | 1.33 | 7.11E−05 | 4.29E−04 | C6orf1 | 1.19 | 8.56E−04 | 2.51E−03 |
| NCAM1 | 6.18 | 4.02E−07 | 2.40E−05 | FSCN1 | 1.22 | 7.14E−05 | 4.30E−04 | PLEC | 1.21 | 8.56E−04 | 2.51E−03 |
| ABHD2 | 1.95 | 4.44E−07 | 2.46E−05 | SOX10 | 1.30 | 7.15E−05 | 4.30E−04 | ARL4C | 1.49 | 8.60E−04 | 2.52E−03 |
| ABR | 1.63 | 4.46E−07 | 2.46E−05 | SERINCS | 2.02 | 7.19E−05 | 4.31E−04 | GOLT1B | 1.27 | 8.60E−04 | 2.52E−03 |
| BRINP2 | 14.54 | 4.41E−07 | 2.46E−05 | PPP1R26 | 1.29 | 7.26E−05 | 4.33E−04 | STT3A | 1.08 | 8.60E−04 | 2.52E−03 |
| CCL3 | 26.58 | 4.30E−07 | 2.46E−05 | KCNN4 | 1.69 | 7.27E−05 | 4.34E−04 | ZCCHC6 | 1.31 | 8.62E−04 | 2.52E−03 |
| FYN | 1.65 | 4.48E−07 | 2.46E−05 | RAB11FIP1 | 1.47 | 7.48E−05 | 4.44E−04 | KMT2D | 1.09 | 8.63E−04 | 2.52E−03 |
| NMNAT2 | 2.95 | 4.47E−07 | 2.46E−05 | LIMS1 | 1.28 | 7.50E−05 | 4.44E−04 | POPDC3 | 1.30 | 8.67E−04 | 2.53E−03 |
| TBX2 | 3.15 | 4.39E−07 | 2.46E−05 | PLD3 | 1.53 | 7.50E−05 | 4.44E−04 | YAP1 | 1.25 | 8.68E−04 | 2.53E−03 |
| ATOX1 | 1.90 | 4.62E−07 | 2.47E−05 | LOC101929959 | 1.14 | 7.55E−05 | 4.46E−04 | ARF4 | 1.17 | 8.77E−04 | 2.56E−03 |
| FHL2 | 2.54 | 4.63E−07 | 2.47E−05 | SH2D2A | 3.93 | 7.61E−05 | 4.48E−04 | TRIM11 | 1.15 | 8.84E−04 | 2.57E−03 |
| NT5E | 2.78 | 4.65E−07 | 2.47E−05 | MARVELD1 | 1.38 | 7.65E−05 | 4.50E−04 | MAPKAPK2 | 1.26 | 8.84E−04 | 2.57E−03 |
| ULK1 | 3.14 | 4.60E−07 | 2.47E−05 | SCAF11 | 1.09 | 7.70E−05 | 4.52E−04 | RANGAP1 | 1.12 | 8.93E−04 | 2.59E−03 |
| CDCP1 | 13.40 | 4.68E−07 | 2.47E−05 | ZFYVE16 | 1.71 | 7.76E−05 | 4.55E−04 | NTM | 1.81 | 8.95E−04 | 2.60E−03 |
| PFDN2 | 1.41 | 4.76E−07 | 2.50E−05 | DPP3 | 1.23 | 7.80E−05 | 4.57E−04 | STUB1 | 1.14 | 8.96E−04 | 2.60E−03 |
| ADAM19 | 2.25 | 4.84E−07 | 2.51E−05 | SLC52A2 | 1.38 | 7.80E−05 | 4.57E−04 | LAMB2 | 1.25 | 8.97E−04 | 2.60E−03 |
| LYPD1 | 3.41 | 4.97E−07 | 2.53E−05 | DSG2 | 1.26 | 7.83E−05 | 4.58E−04 | FAM222B | 1.17 | 9.00E−04 | 2.61E−03 |
| MYL6 | 1.58 | 4.94E−07 | 2.53E−05 | SRPK1 | 1.07 | 7.91E−05 | 4.61E−04 | ENC1 | 1.15 | 9.02E−04 | 2.61E−03 |
| MET | 1.88 | 5.12E−07 | 2.56E−05 | GNB1 | 1.19 | 7.93E−05 | 4.61E−04 | FBXO28 | 1.20 | 9.03E−04 | 2.62E−03 |
| PKIA | 3.71 | 5.07E−07 | 2.56E−05 | PDGFC | 2.62 | 7.95E−05 | 4.62E−04 | IDUA | 1.52 | 9.04E−04 | 2.62E−03 |
| S100A2 | 3.12 | 5.24E−07 | 2.60E−05 | FAM89B | 1.49 | 8.05E−05 | 4.66E−04 | DYNLRB1 | 1.10 | 9.11E−04 | 2.63E−03 |
| PTGFRN | 2.01 | 5.52E−07 | 2.68E−05 | PRKCDBP | 2.28 | 8.05E−05 | 4.66E−04 | NORAD | 1.13 | 9.37E−04 | 2.70E−03 |
| LPXN | 3.52 | 5.61E−07 | 2.68E−05 | SLC35C1 | 1.51 | 8.12E−05 | 4.68E−04 | FMN2 | 1.59 | 9.43E−04 | 2.71E−03 |
| PLA2G4A | 8.02 | 5.58E−07 | 2.68E−05 | TATDN2 | 1.27 | 8.17E−05 | 4.71E−04 | TAX1BP3 | 1.23 | 9.43E−04 | 2.71E−03 |
| DSP | 3.92 | 5.79E−07 | 2.71E−05 | PARP3 | 1.51 | 8.18E−05 | 4.71E−04 | PNPLA2 | 1.14 | 9.43E−04 | 2.71E−03 |
| SLAMF9 | 16.53 | 6.00E−07 | 2.78E−05 | PPTC7 | 1.42 | 8.22E−05 | 4.72E−04 | HIST1H2AC | 1.97 | 9.47E−04 | 2.72E−03 |
| LCE1E | 290.24 | 6.29E−07 | 2.84E−05 | TLE3 | 1.38 | 8.28E−05 | 4.75E−04 | SAGE1 | 1.25 | 9.48E−04 | 2.72E−03 |
| NFKB2 | 2.35 | 6.28E−07 | 2.84E−05 | IGSF8 | 1.90 | 8.30E−05 | 4.75E−04 | NOV | 1.63 | 9.51E−04 | 2.73E−03 |
| TIMP3 | 2.06 | 6.27E−07 | 2.84E−05 | AES | 1.25 | 8.38E−05 | 4.79E−04 | LOC102723728 | 1.36 | 9.55E−04 | 2.74E−03 |
| MPZL1 | 1.46 | 6.34E−07 | 2.85E−05 | ARRDC2 | 1.38 | 8.45E−05 | 4.83E−04 | TMEM64 | 1.27 | 9.57E−04 | 2.74E−03 |
| LGALS1 | 1.30 | 6.37E−07 | 2.85E−05 | IMPDH1 | 1.28 | 8.48E−05 | 4.84E−04 | XBP1 | 1.15 | 9.66E−04 | 2.76E−03 |
| TIMP1 | 4.53 | 6.45E−07 | 2.87E−05 | AKR1C2 | 2.34 | 8.56E−05 | 4.87E−04 | REEP5 | 1.19 | 9.70E−04 | 2.77E−03 |
| CXCL8 | 8.05 | 6.64E−07 | 2.93E−05 | PORCN | 2.11 | 8.57E−05 | 4.88E−04 | RELT | 1.52 | 9.70E−04 | 2.77E−03 |
| WSCD1 | 6.36 | 6.76E−07 | 2.95E−05 | FOXN2 | 1.59 | 8.63E−05 | 4.90E−04 | RPRD1A | 1.50 | 9.77E−04 | 2.79E−03 |
| KCNS3 | 3.22 | 6.82E−07 | 2.97E−05 | FAM107B | 2.32 | 8.70E−05 | 4.93E−04 | CHMP3 | 1.24 | 9.78E−04 | 2.79E−03 |
| A4GALT | 6.11 | 6.97E−07 | 3.00E−05 | PICALM | 1.30 | 8.78E−05 | 4.96E−04 | PGPEP1 | 1.43 | 9.86E−04 | 2.81E−03 |
| DUSP1 | 10.47 | 6.96E−07 | 3.00E−05 | GALNT7 | 1.53 | 8.79E−05 | 4.97E−04 | HM13 | 1.10 | 9.96E−04 | 2.84E−03 |
| BIRC2 | 2.89 | 7.23E−07 | 3.07E−05 | LOC105372663 | 6.01 | 8.83E−05 | 4.98E−04 | YIPF5 | 1.26 | 9.98E−04 | 2.84E−03 |
| LIF | 4.55 | 7.21E−07 | 3.07E−05 | TBX3 | 1.36 | 8.97E−05 | 5.05E−04 | PLPP5 | 1.32 | 1.00E−03 | 2.85E−03 |
| ANXA2 | 1.58 | 7.31E−07 | 3.07E−05 | VPS9D1 | 1.25 | 8.98E−05 | 5.05E−04 | EPN2 | 1.29 | 1.00E−03 | 2.85E−03 |
| HIVEP2 | 2.16 | 7.42E−07 | 3.09E−05 | HIGD1A | 1.20 | 9.02E−05 | 5.07E−04 | SHARPIN | 1.23 | 1.01E−03 | 2.86E−03 |
| FN1 | 1.36 | 7.50E−07 | 3.10E−05 | RELA | 1.39 | 9.06E−05 | 5.08E−04 | SDE2 | 1.21 | 1.01E−03 | 2.87E−03 |
| USP43 | 6.71 | 7.66E−07 | 3.12E−05 | RRAS | 1.66 | 9.07E−05 | 5.08E−04 | ABAT | 1.51 | 1.01E−03 | 2.87E−03 |
| AKAP12 | 1.74 | 7.74E−07 | 3.13E−05 | TTC14 | 1.31 | 9.09E−05 | 5.08E−04 | POGK | 1.24 | 1.01E−03 | 2.87E−03 |
| S100A13 | 1.54 | 7.96E−07 | 3.17E−05 | CD82 | 1.95 | 9.13E−05 | 5.10E−04 | DAPK3 | 1.23 | 1.02E−03 | 2.87E−03 |
| LCE3E | 106.45 | 8.10E−07 | 3.19E−05 | TMEM8A | 1.69 | 9.13E−05 | 5.10E−04 | PITPNM2 | 1.56 | 1.02E−03 | 2.87E−03 |
| BMP2 | 12.05 | 8.50E−07 | 3.30E−05 | GLIS3 | 1.68 | 9.14E−05 | 5.10E−04 | ADGRES | 1.17 | 1.02E−03 | 2.88E−03 |
| BIRC3 | 10.78 | 8.77E−07 | 3.39E−05 | ATP6V1A | 1.17 | 9.16E−05 | 5.11E−04 | NADSYN1 | 1.38 | 1.02E−03 | 2.88E−03 |
| MBOAT2 | 3.19 | 8.95E−07 | 3.43E−05 | RAB7A | 1.26 | 9.23E−05 | 5.14E−04 | EEF2KMT | 1.25 | 1.03E−03 | 2.89E−03 |
| AGTRAP | 1.57 | 9.66E−07 | 3.63E−05 | LOC101927120 | 12.44 | 9.24E−05 | 5.14E−04 | TMEM189 | 1.14 | 1.03E−03 | 2.90E−03 |
| ETHE1 | 3.10 | 9.66E−07 | 3.63E−05 | MSL2 | 1.18 | 9.27E−05 | 5.15E−04 | TRPM4 | 1.57 | 1.03E−03 | 2.91E−03 |
| KLF7 | 3.68 | 9.90E−07 | 3.66E−05 | CRCT1 | 15.56 | 9.29E−05 | 5.16E−04 | C14orf159 | 1.62 | 1.04E−03 | 2.93E−03 |
| NFASC | 3.74 | 9.89E−07 | 3.66E−05 | H6PD | 1.28 | 9.29E−05 | 5.16E−04 | RNF13 | 1.15 | 1.04E−03 | 2.93E−03 |
| CST1 | 186.40 | 1.06E−06 | 3.86E−05 | UCA1 | 2.72 | 9.37E−05 | 5.20E−04 | WFS1 | 1.26 | 1.05E−03 | 2.94E−03 |
| EPAS1 | 10.90 | 1.07E−06 | 3.88E−05 | TCF7L2 | 1.51 | 9.53E−05 | 5.27E−04 | SLC9B2 | 1.32 | 1.05E−03 | 2.95E−03 |
| FAM19A2 | 7.06 | 1.09E−06 | 3.92E−05 | AP1B1 | 1.18 | 9.55E−05 | 5.27E−04 | ZNF707 | 1.16 | 1.05E−03 | 2.95E−03 |
| ANGPTL2 | 3.13 | 1.11E−06 | 3.95E−05 | FBN1 | 1.38 | 9.60E−05 | 5.29E−04 | LMF2 | 1.41 | 1.05E−03 | 2.95E−03 |
| OPRL1 | 18.22 | 1.13E−06 | 3.98E−05 | FEM1C | 1.30 | 9.62E−05 | 5.29E−04 | LOC100507377 | 1.33 | 1.06E−03 | 2.97E−03 |
| GEM | 4.40 | 1.19E−06 | 4.10E−05 | 10-Sep | 1.30 | 9.61E−05 | 5.29E−04 | TSPAN3 | 1.32 | 1.06E−03 | 2.97E−03 |
| MYADM | 1.68 | 1.20E−06 | 4.11E−05 | TM4SF19 | 1.71 | 9.60E−05 | 5.29E−04 | DENNDSA | 1.29 | 1.07E−03 | 2.98E−03 |
| STC1 | 5.09 | 1.20E−06 | 4.11E−05 | CYFIP2 | 1.98 | 9.62E−05 | 5.29E−04 | PPP2R5B | 1.29 | 1.07E−03 | 3.00E−03 |
| ALDH1A3 | 1.30 | 1.23E−06 | 4.17E−05 | IRS2 | 1.36 | 9.63E−05 | 5.29E−04 | CDC37 | 1.07 | 1.08E−03 | 3.00E−03 |
| PITPNC1 | 3.14 | 1.28E−06 | 4.28E−05 | HS3ST3B1 | 5.60 | 9.66E−05 | 5.30E−04 | TSHZ1 | 1.25 | 1.08E−03 | 3.00E−03 |
| TMBIM1 | 2.26 | 1.28E−06 | 4.28E−05 | NFE2L1 | 1.14 | 9.82E−05 | 5.37E−04 | ATP6V0B | 1.15 | 1.09E−03 | 3.02E−03 |
| SPANXB1 | 3.12 | 1.29E−06 | 4.29E−05 | RNF215 | 1.57 | 9.82E−05 | 5.37E−04 | TCEAL1 | 2.07 | 1.10E−03 | 3.04E−03 |
| IFNGR1 | 2.14 | 1.31E−06 | 4.29E−05 | VIM | 1.18 | 9.83E−05 | 5.37E−04 | NECAP2 | 1.23 | 1.12E−03 | 3.09E−03 |
| TPM4 | 1.42 | 1.30E−06 | 4.29E−05 | CYTH2 | 1.31 | 9.86E−05 | 5.37E−04 | TMEM63B | 1.28 | 1.12E−03 | 3.09E−03 |
| CIT | 2.60 | 1.38E−06 | 4.42E−05 | TRPS1 | 1.66 | 9.89E−05 | 5.38E−04 | ZFP36 | 1.52 | 1.12E−03 | 3.10E−03 |
| NRP2 | 1.96 | 1.36E−06 | 4.42E−05 | STARD13 | 1.70 | 1.00E−04 | 5.45E−04 | CDR2L | 1.51 | 1.13E−03 | 3.11E−03 |
| SPOCK1 | 2.78 | 1.36E−06 | 4.42E−05 | STEAP1B | 1.42 | 1.01E−04 | 5.48E−04 | MAPKAP1 | 1.18 | 1.13E−03 | 3.11E−03 |
| ST3GAL1 | 2.10 | 1.37E−06 | 4.42E−05 | SPAG9 | 1.44 | 1.01E−04 | 5.48E−04 | EMC10 | 1.48 | 1.13E−03 | 3.12E−03 |
| ITGAS | 2.24 | 1.39E−06 | 4.43E−05 | SYVN1 | 1.38 | 1.01E−04 | 5.48E−04 | SDF2 | 1.16 | 1.13E−03 | 3.13E−03 |
| PERP | 1.86 | 1.40E−06 | 4.43E−05 | ARAP1 | 1.33 | 1.02E−04 | 5.51E−04 | EEF1A1 | 1.08 | 1.14E−03 | 3.13E−03 |
| TMBIM6 | 1.16 | 1.39E−06 | 4.43E−05 | MAFG | 1.50 | 1.02E−04 | 5.53E−04 | B3GAT3 | 1.22 | 1.15E−03 | 3.15E−03 |
| WNT9A | 8.21 | 1.40E−06 | 4.43E−05 | GADD45B | 1.80 | 1.02E−04 | 5.54E−04 | CD164 | 1.20 | 1.16E−03 | 3.18E−03 |
| FAT3 | 5.84 | 1.46E−06 | 4.60E−05 | DMKN | 2.57 | 1.03E−04 | 5.54E−04 | DPP9 | 1.11 | 1.16E−03 | 3.18E−03 |
| HMOX1 | 4.62 | 1.49E−06 | 4.62E−05 | C7orf43 | 1.36 | 1.03E−04 | 5.54E−04 | PGRMC1 | 1.14 | 1.16E−03 | 3.19E−03 |
| LCE1C | 79.96 | 1.49E−06 | 4.62E−05 | DUSP7 | 1.29 | 1.04E−04 | 5.60E−04 | WDFY2 | 1.61 | 1.16E−03 | 3.19E−03 |
| RNF19B | 3.91 | 1.49E−06 | 4.62E−05 | UBA6-AS1 | 1.78 | 1.05E−04 | 5.60E−04 | PPP4R1 | 1.42 | 1.18E−03 | 3.22E−03 |
| TOMM34 | 1.90 | 1.49E−06 | 4.62E−05 | GALNT6 | 2.73 | 1.05E−04 | 5.61E−04 | FAM199X | 1.12 | 1.18E−03 | 3.23E−03 |
| ADIPOR1 | 1.45 | 1.55E−06 | 4.67E−05 | KCNC4 | 4.83 | 1.06E−04 | 5.64E−04 | BCAS3 | 1.67 | 1.18E−03 | 3.23E−03 |
| BPGM | 3.63 | 1.53E−06 | 4.67E−05 | PLOD1 | 1.26 | 1.06E−04 | 5.64E−04 | C6orf89 | 1.17 | 1.18E−03 | 3.23E−03 |
| TM4SF1 | 3.11 | 1.56E−06 | 4.67E−05 | SZT2 | 1.16 | 1.06E−04 | 5.64E−04 | NDN | 1.25 | 1.18E−03 | 3.23E−03 |
| DIP2B | 3.61 | 1.60E−06 | 4.74E−05 | MMP9 | 6.53 | 1.06E−04 | 5.65E−04 | GPN1 | 1.26 | 1.19E−03 | 3.24E−03 |
| CD93 | 11.75 | 1.62E−06 | 4.75E−05 | CALCOCO1 | 1.42 | 1.07E−04 | 5.65E−04 | PAQR4 | 1.20 | 1.19E−03 | 3.24E−03 |
| SREK1IP1 | 2.09 | 1.61E−06 | 4.75E−05 | UBL3 | 1.52 | 1.07E−04 | 5.65E−04 | SLCO4A1-AS1 | 1.47 | 1.19E−03 | 3.24E−03 |
| STIM1 | 2.05 | 1.62E−06 | 4.75E−05 | SLC20A1 | 1.19 | 1.08E−04 | 5.68E−04 | UHMK1 | 1.19 | 1.19E−03 | 3.24E−03 |
| TLN2 | 3.95 | 1.63E−06 | 4.75E−05 | MLXIP | 1.40 | 1.09E−04 | 5.74E−04 | YIPF3 | 1.12 | 1.19E−03 | 3.24E−03 |
| GRB10 | 3.48 | 1.66E−06 | 4.77E−05 | ABI1 | 1.39 | 1.09E−04 | 5.75E−04 | PAFAH1B2 | 1.25 | 1.19E−03 | 3.24E−03 |
| GPRC5A | 1.95 | 1.70E−06 | 4.78E−05 | TMEM87A | 1.37 | 1.10E−04 | 5.76E−04 | SMAD7 | 2.17 | 1.20E−03 | 3.27E−03 |
| LRIG1 | 4.18 | 1.69E−06 | 4.78E−05 | CEP170B | 1.57 | 1.10E−04 | 5.78E−04 | C11orf68 | 1.39 | 1.21E−03 | 3.28E−03 |
| PFKFB4 | 2.37 | 1.68E−06 | 4.78E−05 | LOC102724122 | 1.61 | 1.10E−04 | 5.78E−04 | C9orf72 | 1.70 | 1.21E−03 | 3.28E−03 |
| PLAUR | 2.45 | 1.67E−06 | 4.78E−05 | ITM2B | 1.11 | 1.11E−04 | 5.78E−04 | TPM3 | 1.11 | 1.21E−03 | 3.28E−03 |
| HMGA2 | 2.09 | 1.71E−06 | 4.79E−05 | HES1 | 1.72 | 1.11E−04 | 5.80E−04 | TRIM35 | 1.55 | 1.22E−03 | 3.29E−03 |
| ACPP | 24.20 | 1.72E−06 | 4.80E−05 | MAGEA10 | 1.69 | 1.11E−04 | 5.81E−04 | COX1 | 1.16 | 1.24E−03 | 3.34E−03 |
| TMSB10 | 1.60 | 1.73E−06 | 4.82E−05 | CCNDBP1 | 1.58 | 1.11E−04 | 5.81E−04 | RNASEK | 1.31 | 1.24E−03 | 3.35E−03 |
| MYD88 | 3.03 | 1.74E−06 | 4.83E−05 | ZNF267 | 1.85 | 1.12E−04 | 5.82E−04 | TMEM134 | 1.42 | 1.25E−03 | 3.37E−03 |
| CCDC71L | 1.84 | 1.76E−06 | 4.85E−05 | B2M | 1.29 | 1.12E−04 | 5.82E−04 | ZFAND6 | 1.20 | 1.26E−03 | 3.39E−03 |
| GALNT9 | 13.43 | 1.78E−06 | 4.85E−05 | AHRR | 1.39 | 1.12E−04 | 5.82E−04 | HLA-F | 1.82 | 1.26E−03 | 3.39E−03 |
| KRTAP2-3 | 6.40 | 1.78E−06 | 4.85E−05 | CALD1 | 1.39 | 1.12E−04 | 5.82E−04 | MEX3C | 1.18 | 1.26E−03 | 3.40E−03 |
| PHLDA1 | 1.58 | 1.77E−06 | 4.85E−05 | SMAP1 | 1.25 | 1.12E−04 | 5.82E−04 | BET1L | 1.12 | 1.27E−03 | 3.41E−03 |
| ACTG1 | 1.35 | 1.78E−06 | 4.85E−05 | MAN2B1 | 1.40 | 1.13E−04 | 5.83E−04 | FBXO11 | 1.19 | 1.29E−03 | 3.45E−03 |
| RGMB | 3.23 | 1.79E−06 | 4.85E−05 | MDGA1 | 5.88 | 1.14E−04 | 5.87E−04 | PCBD1 | 1.30 | 1.29E−03 | 3.46E−03 |
| ACOT9 | 1.92 | 1.81E−06 | 4.89E−05 | FGFR1 | 1.43 | 1.15E−04 | 5.89E−04 | BHLHE40 | 1.39 | 1.29E−03 | 3.46E−03 |
| PTPRZ1 | 33.73 | 1.85E−06 | 4.95E−05 | FLOT2 | 1.38 | 1.15E−04 | 5.89E−04 | GNAI1 | 1.47 | 1.30E−03 | 3.47E−03 |
| TRNP1 | 2.20 | 1.85E−06 | 4.95E−05 | NPDC1 | 1.90 | 1.15E−04 | 5.89E−04 | NDEL1 | 1.22 | 1.32E−03 | 3.51E−03 |
| FEZ1 | 1.50 | 1.97E−06 | 5.14E−05 | CENPM | 1.65 | 1.15E−04 | 5.89E−04 | SMIM19 | 1.23 | 1.32E−03 | 3.52E−03 |
| SMTN | 2.09 | 1.97E−06 | 5.14E−05 | ABLIM1 | 1.93 | 1.15E−04 | 5.91E−04 | UHRF2 | 1.42 | 1.33E−03 | 3.54E−03 |
| C1orf198 | 3.14 | 2.07E−06 | 5.32E−05 | GAK | 1.40 | 1.16E−04 | 5.91E−04 | PTTG1IP | 1.18 | 1.34E−03 | 3.57E−03 |
| KPRP | 54.25 | 2.06E−06 | 5.32E−05 | CTHRC1 | 2.10 | 1.17E−04 | 5.98E−04 | SMARCA2 | 1.27 | 1.34E−03 | 3.57E−03 |
| NUP50 | 1.87 | 2.07E−06 | 5.32E−05 | RPL10 | 1.12 | 1.17E−04 | 5.98E−04 | DPY19L3 | 1.33 | 1.34E−03 | 3.57E−03 |
| IGF2R | 1.16 | 2.17E−06 | 5.42E−05 | TERF2IP | 1.18 | 1.17E−04 | 5.98E−04 | HYAL3 | 1.59 | 1.35E−03 | 3.57E−03 |
| NUAK2 | 7.22 | 2.15E−06 | 5.42E−05 | ZNF275 | 1.62 | 1.17E−04 | 5.98E−04 | HS3ST3A1 | 1.22 | 1.35E−03 | 3.58E−03 |
| SEL1L3 | 2.22 | 2.15E−06 | 5.42E−05 | MYO10 | 1.78 | 1.19E−04 | 6.06E−04 | PRCP | 1.27 | 1.35E−03 | 3.58E−03 |
| GRN | 1.61 | 2.19E−06 | 5.45E−05 | DHRS1 | 1.81 | 1.21E−04 | 6.12E−04 | FAM134C | 1.17 | 1.37E−03 | 3.61E−03 |
| MAP1B | 2.08 | 2.20E−06 | 5.45E−05 | EIF1 | 1.20 | 1.21E−04 | 6.13E−04 | ADO | 1.40 | 1.37E−03 | 3.62E−03 |
| TMCC3 | 2.82 | 2.24E−06 | 5.50E−05 | CTSB | 1.24 | 1.22E−04 | 6.16E−04 | SP100 | 1.47 | 1.37E−03 | 3.62E−03 |
| LINC00941 | 3.78 | 2.28E−06 | 5.53E−05 | SLC2A13 | 2.67 | 1.23E−04 | 6.22E−04 | ZNF444 | 1.20 | 1.37E−03 | 3.62E−03 |
| ATG101 | 1.23 | 2.30E−06 | 5.55E−05 | NOP9 | 1.14 | 1.24E−04 | 6.24E−04 | SLC35F2 | 1.39 | 1.38E−03 | 3.63E−03 |
| S100A11 | 1.85 | 2.36E−06 | 5.67E−05 | ARHGEF4 | 2.31 | 1.24E−04 | 6.26E−04 | NENF | 1.14 | 1.38E−03 | 3.63E−03 |
| JOSD1 | 1.67 | 2.37E−06 | 5.68E−05 | ENTPD7 | 1.57 | 1.25E−04 | 6.27E−04 | EDEM3 | 1.17 | 1.38E−03 | 3.64E−03 |
| GAS6 | 1.81 | 2.39E−06 | 5.70E−05 | KIF1C | 1.15 | 1.25E−04 | 6.27E−04 | SLC4A3 | 1.39 | 1.38E−03 | 3.64E−03 |
| ARPC1B | 1.61 | 2.41E−06 | 5.74E−05 | RAB6A | 1.33 | 1.25E−04 | 6.27E−04 | EDEM2 | 1.17 | 1.39E−03 | 3.65E−03 |
| LRRFIP1 | 1.73 | 2.48E−06 | 5.82E−05 | REST | 1.27 | 1.25E−04 | 6.28E−04 | MYO1E | 1.12 | 1.39E−03 | 3.66E−03 |
| MVP | 1.81 | 2.48E−06 | 5.82E−05 | ACTB | 1.08 | 1.25E−04 | 6.29E−04 | FAM214A | 1.23 | 1.39E−03 | 3.66E−03 |
| PSAP | 1.61 | 2.48E−06 | 5.82E−05 | MAP3K4 | 1.43 | 1.26E−04 | 6.30E−04 | INSIG1 | 1.21 | 1.39E−03 | 3.66E−03 |
| ZNF536 | 3.88 | 2.46E−06 | 5.82E−05 | POLD4 | 1.48 | 1.27E−04 | 6.32E−04 | ELK4 | 1.31 | 1.40E−03 | 3.66E−03 |
| MAP1S | 1.79 | 2.49E−06 | 5.83E−05 | VAT1L | 1.78 | 1.27E−04 | 6.32E−04 | ARPP19 | 1.15 | 1.40E−03 | 3.67E−03 |
| ARSB | 5.08 | 2.52E−06 | 5.85E−05 | CSRP1 | 1.33 | 1.27E−04 | 6.34E−04 | KRT15 | 1.38 | 1.41E−03 | 3.69E−03 |
| HGS | 1.41 | 2.52E−06 | 5.85E−05 | ALDH9A1 | 1.23 | 1.27E−04 | 6.34E−04 | LGALS3BP | 1.14 | 1.41E−03 | 3.69E−03 |
| PDLIM7 | 2.22 | 2.54E−06 | 5.86E−05 | PDLIM4 | 1.24 | 1.27E−04 | 6.35E−04 | ARHGAP31 | 1.39 | 1.42E−03 | 3.71E−03 |
| HLA-C | 1.34 | 2.55E−06 | 5.86E−05 | CAP1 | 1.18 | 1.28E−04 | 6.36E−04 | CSTB | 1.26 | 1.43E−03 | 3.75E−03 |
| CDK5R1 | 1.75 | 2.63E−06 | 5.95E−05 | SEC61A1 | 1.14 | 1.28E−04 | 6.37E−04 | FAM234A | 1.17 | 1.44E−03 | 3.77E−03 |
| PRRT4 | 15.52 | 2.66E−06 | 5.99E−05 | GALNT1 | 1.32 | 1.28E−04 | 6.37E−04 | KDELC1 | 1.31 | 1.45E−03 | 3.78E−03 |
| CCDC80 | 2.95 | 2.69E−06 | 6.02E−05 | GM2A | 1.41 | 1.28E−04 | 6.37E−04 | CMAS | 1.23 | 1.45E−03 | 3.78E−03 |
| UBA1 | 1.17 | 2.74E−06 | 6.08E−05 | ISCA1 | 1.48 | 1.30E−04 | 6.42E−04 | PDPK1 | 1.19 | 1.47E−03 | 3.82E−03 |
| WLS | 1.53 | 2.77E−06 | 6.11E−05 | NDFIP1 | 1.23 | 1.30E−04 | 6.42E−04 | SLC25A4 | 1.14 | 1.47E−03 | 3.82E−03 |
| GIPC1 | 1.30 | 2.79E−06 | 6.13E−05 | PRR12 | 1.12 | 1.31E−04 | 6.46E−04 | EIF2AK1 | 1.16 | 1.48E−03 | 3.85E−03 |
| BLACAT1 | 2.56 | 2.80E−06 | 6.14E−05 | MAFK | 1.66 | 1.31E−04 | 6.47E−04 | MGC72080 | 1.31 | 1.48E−03 | 3.85E−03 |
| USP11 | 1.53 | 2.84E−06 | 6.19E−05 | RASAL2 | 1.22 | 1.32E−04 | 6.50E−04 | SIK3 | 1.32 | 1.48E−03 | 3.85E−03 |
| GALNT12 | 9.13 | 2.85E−06 | 6.19E−05 | ANKRA2 | 1.63 | 1.34E−04 | 6.56E−04 | UHRF1BP1L | 1.22 | 1.48E−03 | 3.85E−03 |
| CD9 | 2.43 | 2.89E−06 | 6.19E−05 | COPZ2 | 1.62 | 1.34E−04 | 6.57E−04 | WIPI1 | 1.36 | 1.49E−03 | 3.86E−03 |
| CUEDC1 | 1.41 | 2.87E−06 | 6.19E−05 | FRYL | 1.38 | 1.34E−04 | 6.57E−04 | STXBP3 | 1.25 | 1.50E−03 | 3.90E−03 |
| LCE1D | 183.66 | 2.88E−06 | 6.19E−05 | PRKCD | 1.36 | 1.35E−04 | 6.61E−04 | CD81 | 1.18 | 1.51E−03 | 3.91E−03 |
| NPC1 | 1.52 | 2.90E−06 | 6.19E−05 | SLC1A4 | 1.42 | 1.36E−04 | 6.66E−04 | PTRH1 | 1.50 | 1.53E−03 | 3.95E−03 |
| PNP | 1.84 | 2.89E−06 | 6.19E−05 | CORO1B | 1.22 | 1.36E−04 | 6.66E−04 | BLOC1S6 | 1.13 | 1.53E−03 | 3.96E−03 |
| ND3 | 1.26 | 2.91E−06 | 6.19E−05 | C7orf73 | 1.34 | 1.37E−04 | 6.68E−04 | IFI27L2 | 1.59 | 1.54E−03 | 3.97E−03 |
| G3BP1 | 1.23 | 2.96E−06 | 6.25E−05 | ICAM1 | 1.43 | 1.37E−04 | 6.68E−04 | DYRK3 | 1.16 | 1.54E−03 | 3.97E−03 |
| ID1 | 2.35 | 2.96E−06 | 6.25E−05 | PIKFYVE | 1.36 | 1.38E−04 | 6.72E−04 | SNX30 | 1.29 | 1.55E−03 | 4.00E−03 |
| CXCL1 | 3.12 | 3.01E−06 | 6.32E−05 | DNAJB9 | 1.67 | 1.40E−04 | 6.79E−04 | SIRPB1 | 2.12 | 1.55E−03 | 4.00E−03 |
| MYO5A | 2.62 | 3.03E−06 | 6.34E−05 | PLPP4 | 1.87 | 1.40E−04 | 6.79E−04 | HTT | 1.11 | 1.56E−03 | 4.01E−03 |
| CCND1 | 1.23 | 3.05E−06 | 6.38E−05 | KLF6 | 1.82 | 1.41E−04 | 6.81E−04 | GZF1 | 1.80 | 1.56E−03 | 4.01E−03 |
| FBXO32 | 2.47 | 3.08E−06 | 6.38E−05 | LTBP3 | 1.20 | 1.41E−04 | 6.81E−04 | RAB18 | 1.31 | 1.57E−03 | 4.05E−03 |
| MELTF | 1.81 | 3.08E−06 | 6.38E−05 | WBP1L | 1.42 | 1.41E−04 | 6.82E−04 | UBL5 | 1.07 | 1.59E−03 | 4.08E−03 |
| PIEZO1 | 1.60 | 3.09E−06 | 6.38E−05 | ELK3 | 1.07 | 1.42E−04 | 6.82E−04 | ATG13 | 1.05 | 1.60E−03 | 4.09E−03 |
| CAPN1 | 1.58 | 3.13E−06 | 6.44E−05 | PTPRM | 2.41 | 1.42E−04 | 6.82E−04 | DNASE1L1 | 1.54 | 1.60E−03 | 4.10E−03 |
| AREG | 9.92 | 3.14E−06 | 6.45E−05 | TUBB3 | 1.27 | 1.42E−04 | 6.82E−04 | CLPTM1 | 1.10 | 1.61E−03 | 4.10E−03 |
| ARIDSA | 3.00 | 3.14E−06 | 6.45E−05 | BCAS4 | 1.51 | 1.42E−04 | 6.84E−04 | LKAAEAR1 | 7.81 | 1.60E−03 | 4.10E−03 |
| SLC39A6 | 1.74 | 3.20E−06 | 6.54E−05 | HIST1H2BK | 2.47 | 1.43E−04 | 6.84E−04 | KCTD11 | 1.52 | 1.62E−03 | 4.12E−03 |
| RHOB | 1.83 | 3.23E−06 | 6.57E−05 | GRINA | 1.23 | 1.44E−04 | 6.89E−04 | FAM50A | 1.12 | 1.62E−03 | 4.13E−03 |
| PARP4 | 1.31 | 3.25E−06 | 6.59E−05 | ANKRD36BP1 | 1.78 | 1.44E−04 | 6.89E−04 | RCAN3 | 1.35 | 1.62E−03 | 4.13E−03 |
| BTG1 | 1.49 | 3.28E−06 | 6.63E−05 | PHKA1 | 1.52 | 1.44E−04 | 6.90E−04 | TEX30 | 1.51 | 1.63E−03 | 4.15E−03 |
| SORBS2 | 2.08 | 3.31E−06 | 6.64E−05 | PTHLH | 2.23 | 1.45E−04 | 6.91E−04 | LOC100506548 | 1.28 | 1.63E−03 | 4.15E−03 |
| CYBA | 1.41 | 3.35E−06 | 6.68E−05 | PSEN2 | 1.55 | 1.45E−04 | 6.94E−04 | ACTR3 | 1.12 | 1.65E−03 | 4.18E−03 |
| C3orf67 | 3.51 | 3.39E−06 | 6.69E−05 | GPR161 | 1.80 | 1.46E−04 | 6.95E−04 | MAVS | 1.11 | 1.65E−03 | 4.18E−03 |
| PYGB | 1.29 | 3.38E−06 | 6.69E−05 | TBC1D9B | 1.11 | 1.48E−04 | 7.05E−04 | DUSP23 | 1.23 | 1.66E−03 | 4.20E−03 |
| TWF1 | 1.88 | 3.39E−06 | 6.69E−05 | TBC1D9 | 1.88 | 1.49E−04 | 7.08E−04 | SLCO4A1 | 1.28 | 1.66E−03 | 4.22E−03 |
| LCE3D | 107.08 | 3.43E−06 | 6.75E−05 | VPS13C | 1.31 | 1.49E−04 | 7.09E−04 | RPS6KA4 | 1.18 | 1.67E−03 | 4.24E−03 |
| PARM1 | 5.09 | 3.46E−06 | 6.77E−05 | RECQL | 1.21 | 1.50E−04 | 7.10E−04 | BICD1 | 1.23 | 1.68E−03 | 4.24E−03 |
| IL1A | 4.03 | 3.69E−06 | 7.09E−05 | TNFRSF25 | 2.66 | 1.50E−04 | 7.12E−04 | PCGF3 | 1.09 | 1.68E−03 | 4.25E−03 |
| OXSR1 | 1.62 | 3.71E−06 | 7.10E−05 | RNF149 | 3.14 | 1.50E−04 | 7.12E−04 | ZNF598 | 1.16 | 1.68E−03 | 4.25E−03 |
| UCN2 | 3.37 | 3.77E−06 | 7.17E−05 | IL11 | 1.55 | 1.51E−04 | 7.14E−04 | WDR13 | 1.22 | 1.71E−03 | 4.30E−03 |
| HMCN2 | 35.09 | 3.88E−06 | 7.29E−05 | ZCCHC14 | 1.53 | 1.52E−04 | 7.17E−04 | NOMO1 | 1.15 | 1.71E−03 | 4.31E−03 |
| PMP22 | 1.36 | 4.12E−06 | 7.62E−05 | ANKS6 | 1.49 | 1.52E−04 | 7.18E−04 | ITPKB | 1.44 | 1.71E−03 | 4.31E−03 |
| FKBP10 | 1.41 | 4.18E−06 | 7.69E−05 | LINC00467 | 1.22 | 1.53E−04 | 7.23E−04 | SETD1B | 1.11 | 1.73E−03 | 4.34E−03 |
| PROSER2 | 3.15 | 4.21E−06 | 7.71E−05 | IFT80 | 1.52 | 1.54E−04 | 7.24E−04 | TRPC4AP | 1.07 | 1.73E−03 | 4.34E−03 |
| CDK17 | 2.28 | 4.23E−06 | 7.73E−05 | SERGEF | 1.49 | 1.54E−04 | 7.26E−04 | SLC6A8 | 1.05 | 1.73E−03 | 4.34E−03 |
| IRAK2 | 3.73 | 4.32E−06 | 7.81E−05 | FAM134A | 1.34 | 1.56E−04 | 7.33E−04 | TGOLN2 | 1.05 | 1.73E−03 | 4.35E−03 |
| FKBP1A | 1.28 | 4.47E−06 | 7.96E−05 | CNKSR3 | 1.69 | 1.58E−04 | 7.37E−04 | DECR1 | 1.44 | 1.74E−03 | 4.36E−03 |
| HIST2H2AA3 | 2.63 | 4.46E−06 | 7.96E−05 | TACC1 | 1.25 | 1.58E−04 | 7.37E−04 | DOCK7 | 1.34 | 1.74E−03 | 4.37E−03 |
| HIST2H2AA4 | 2.63 | 4.46E−06 | 7.96E−05 | GDNF | 2.20 | 1.58E−04 | 7.38E−04 | NCSTN | 1.12 | 1.75E−03 | 4.37E−03 |
| IKZF2 | 6.86 | 4.50E−06 | 7.98E−05 | KRT14 | 1.21 | 1.58E−04 | 7.38E−04 | GMIP | 1.28 | 1.75E−03 | 4.38E−03 |
| SYNJ2 | 2.13 | 4.50E−06 | 7.98E−05 | SGK1 | 1.40 | 1.59E−04 | 7.39E−04 | ZNFX1 | 1.25 | 1.76E−03 | 4.40E−03 |
| TAC1 | 20.39 | 4.49E−06 | 7.98E−05 | HGSNAT | 1.30 | 1.59E−04 | 7.40E−04 | AHNAK | 1.16 | 1.77E−03 | 4.41E−03 |
| NDRG1 | 1.73 | 4.54E−06 | 8.01E−05 | SPSB1 | 1.96 | 1.60E−04 | 7.42E−04 | NUDT22 | 1.24 | 1.77E−03 | 4.42E−03 |
| ETS1 | 2.42 | 4.67E−06 | 8.19E−05 | SYNGR2 | 1.37 | 1.60E−04 | 7.43E−04 | PELI1 | 1.40 | 1.77E−03 | 4.42E−03 |
| LOC100996740 | 2.10 | 4.69E−06 | 8.19E−05 | PBX3 | 1.24 | 1.60E−04 | 7.44E−04 | MESDC1 | 1.40 | 1.78E−03 | 4.42E−03 |
| TRIO | 1.77 | 4.69E−06 | 8.19E−05 | RGP1 | 1.19 | 1.62E−04 | 7.50E−04 | MTAP | 1.06 | 1.78E−03 | 4.43E−03 |
| RNF144A | 1.72 | 4.82E−06 | 8.31E−05 | TMEM54 | 1.57 | 1.62E−04 | 7.51E−04 | AIG1 | 1.59 | 1.79E−03 | 4.45E−03 |
| TRPV4 | 1.89 | 4.81E−06 | 8.31E−05 | UBE2Z | 1.27 | 1.63E−04 | 7.54E−04 | UGGT1 | 1.06 | 1.79E−03 | 4.45E−03 |
| PTGS2 | 14.30 | 4.90E−06 | 8.38E−05 | GNAI2 | 1.36 | 1.65E−04 | 7.60E−04 | LINC00622 | 1.75 | 1.80E−03 | 4.47E−03 |
| PRNP | 1.92 | 4.92E−06 | 8.40E−05 | MYL12A | 1.31 | 1.65E−04 | 7.62E−04 | CHTF8 | 1.06 | 1.80E−03 | 4.47E−03 |
| ADGRE2 | 2.23 | 4.94E−06 | 8.43E−05 | TAPBP | 1.51 | 1.65E−04 | 7.62E−04 | CD47 | 1.20 | 1.81E−03 | 4.49E−03 |
| FAS | 2.13 | 4.96E−06 | 8.44E−05 | APOD | 2.35 | 1.66E−04 | 7.63E−04 | MKRN1 | 1.16 | 1.82E−03 | 4.50E−03 |
| ADK | 2.22 | 5.09E−06 | 8.59E−05 | TAF4B | 1.41 | 1.66E−04 | 7.63E−04 | UBE2L6 | 1.22 | 1.84E−03 | 4.53E−03 |
| TRIB1 | 1.61 | 5.09E−06 | 8.59E−05 | NR1D2 | 1.31 | 1.66E−04 | 7.64E−04 | SESN2 | 1.21 | 1.84E−03 | 4.54E−03 |
| BTBD10 | 1.57 | 5.11E−06 | 8.61E−05 | SKAP2 | 1.46 | 1.67E−04 | 7.65E−04 | CPTP | 1.48 | 1.85E−03 | 4.56E−03 |
| RAPH1 | 3.40 | 5.15E−06 | 8.65E−05 | AGPAT2 | 1.35 | 1.67E−04 | 7.67E−04 | TMEM184B | 1.15 | 1.86E−03 | 4.59E−03 |
| PLD5 | 18.93 | 5.18E−06 | 8.67E−05 | SMG7 | 1.26 | 1.68E−04 | 7.71E−04 | PLEKHA3 | 1.27 | 1.88E−03 | 4.63E−03 |
| ITGB1 | 1.33 | 5.24E−06 | 8.68E−05 | MFSD1 | 1.30 | 1.69E−04 | 7.71E−04 | PLBD2 | 1.26 | 1.89E−03 | 4.64E−03 |
| LRP10 | 1.56 | 5.24E−06 | 8.68E−05 | TPT1 | 1.14 | 1.69E−04 | 7.73E−04 | CITED4 | 1.43 | 1.89E−03 | 4.65E−03 |
| DBNDD1 | 2.02 | 5.30E−06 | 8.71E−05 | RNF14 | 1.13 | 1.70E−04 | 7.74E−04 | EXOC2 | 1.17 | 1.90E−03 | 4.67E−03 |
| PLOD3 | 1.24 | 5.40E−06 | 8.78E−05 | ADD2 | 1.59 | 1.70E−04 | 7.75E−04 | ACBD5 | 1.28 | 1.91E−03 | 4.69E−03 |
| RASA3 | 1.66 | 5.39E−06 | 8.78E−05 | NUCB1 | 1.16 | 1.70E−04 | 7.75E−04 | P4HB | 1.05 | 1.92E−03 | 4.71E−03 |
| CORO1C | 1.88 | 5.48E−06 | 8.84E−05 | RNASET2 | 1.45 | 1.71E−04 | 7.77E−04 | TRIM24 | 1.17 | 1.94E−03 | 4.73E−03 |
| MXRA7 | 1.93 | 5.52E−06 | 8.88E−05 | SGPL1 | 1.36 | 1.71E−04 | 7.77E−04 | PTPN12 | 1.12 | 1.98E−03 | 4.81E−03 |
| ADAMTS1 | 2.65 | 5.56E−06 | 8.93E−05 | SLC11A2 | 1.33 | 1.71E−04 | 7.77E−04 | CDK16 | 1.11 | 1.98E−03 | 4.82E−03 |
| SASH1 | 1.79 | 5.67E−06 | 9.09E−05 | PLEKHM1 | 1.33 | 1.71E−04 | 7.78E−04 | ASNS | 1.26 | 1.98E−03 | 4.82E−03 |
| MANBA | 2.24 | 5.70E−06 | 9.11E−05 | FYCO1 | 1.47 | 1.72E−04 | 7.79E−04 | CC2D1B | 1.15 | 1.99E−03 | 4.83E−03 |
| DLGAP4 | 1.83 | 5.76E−06 | 9.19E−05 | AIDA | 1.55 | 1.72E−04 | 7.79E−04 | CRIPT | 1.29 | 1.99E−03 | 4.85E−03 |
| TJP1 | 1.76 | 5.83E−06 | 9.25E−05 | GPAT3 | 1.26 | 1.72E−04 | 7.79E−04 | HEBP2 | 1.49 | 2.01E−03 | 4.88E−03 |
| CAMK1 | 6.99 | 5.93E−06 | 9.35E−05 | TMEM120A | 1.30 | 1.74E−04 | 7.85E−04 | SLC22A4 | 1.58 | 2.02E−03 | 4.90E−03 |
| UBE2R2 | 1.36 | 5.99E−06 | 9.38E−05 | NPR2 | 1.26 | 1.75E−04 | 7.88E−04 | TBC1D19 | 1.65 | 2.05E−03 | 4.95E−03 |
| RPS6KA2 | 3.55 | 6.02E−06 | 9.41E−05 | ARG2 | 2.24 | 1.75E−04 | 7.89E−04 | SNAPC2 | 1.38 | 2.06E−03 | 4.97E−03 |
| DSTYK | 1.45 | 6.20E−06 | 9.53E−05 | CASC10 | 1.83 | 1.75E−04 | 7.89E−04 | HPS6 | 1.24 | 2.06E−03 | 4.98E−03 |
| LYPD3 | 12.82 | 6.18E−06 | 9.53E−05 | OS9 | 1.28 | 1.76E−04 | 7.89E−04 | MAEA | 1.15 | 2.08E−03 | 5.00E−03 |
| HSPG2 | 2.20 | 6.23E−06 | 9.56E−05 | SND1 | 1.18 | 1.76E−04 | 7.92E−04 | MXD4 | 1.39 | 2.08E−03 | 5.01E−03 |
| DCBLD2 | 1.50 | 6.33E−06 | 9.68E−05 | SMARCAL1 | 1.13 | 1.78E−04 | 7.96E−04 | ADCK2 | 1.20 | 2.08E−03 | 5.01E−03 |
| SPRY2 | 1.61 | 6.35E−06 | 9.68E−05 | MAN2B2 | 1.70 | 1.79E−04 | 7.99E−04 | MAFF | 1.42 | 2.09E−03 | 5.02E−03 |
| ZEB1 | 1.48 | 6.35E−06 | 9.68E−05 | MTMR1 | 1.56 | 1.81E−04 | 8.06E−04 | COG3 | 1.31 | 2.09E−03 | 5.03E−03 |
| SATB1 | 2.61 | 6.37E−06 | 9.68E−05 | RGS20 | 2.62 | 1.81E−04 | 8.06E−04 | TFRC | 1.12 | 2.11E−03 | 5.07E−03 |
| GK | 7.46 | 6.51E−06 | 9.85E−05 | MMP16 | 1.27 | 1.82E−04 | 8.08E−04 | MARK2 | 1.09 | 2.11E−03 | 5.08E−03 |
| RAB11FIP5 | 1.97 | 6.52E−06 | 9.85E−05 | CREM | 2.19 | 1.82E−04 | 8.11E−04 | PIP4K2C | 1.24 | 2.12E−03 | 5.09E−03 |
| SH3PXD2B | 1.83 | 6.58E−06 | 9.89E−05 | ADAMTS15 | 2.02 | 1.85E−04 | 8.20E−04 | TLCD1 | 1.39 | 2.12E−03 | 5.09E−03 |
| PLAT | 3.65 | 6.65E−06 | 9.96E−05 | BMP2K | 2.08 | 1.85E−04 | 8.21E−04 | CYSTM1 | 1.58 | 2.12E−03 | 5.10E−03 |
| TRAM2 | 1.57 | 6.71E−06 | 1.00E−04 | KIFAP3 | 1.46 | 1.86E−04 | 8.24E−04 | SLC39A8 | 1.54 | 2.12E−03 | 5.10E−03 |
| ADAMTSL1 | 6.17 | 7.10E−06 | 1.05E−04 | NR1H2 | 1.20 | 1.87E−04 | 8.26E−04 | RTCB | 1.10 | 2.15E−03 | 5.16E−03 |
| PARVB | 1.46 | 7.30E−06 | 1.07E−04 | MGAT4B | 1.26 | 1.87E−04 | 8.26E−04 | LOC107985004 | 1.45 | 2.16E−03 | 5.16E−03 |
| IL13RA1 | 1.90 | 7.37E−06 | 1.08E−04 | GNB5 | 1.14 | 1.89E−04 | 8.32E−04 | PEA15 | 1.05 | 2.16E−03 | 5.16E−03 |
| PRR9 | 587.37 | 7.42E−06 | 1.08E−04 | SERINC1 | 1.33 | 1.89E−04 | 8.33E−04 | PMAIP1 | 1.24 | 2.16E−03 | 5.16E−03 |
| IL4R | 2.76 | 7.46E−06 | 1.08E−04 | LINC00869 | 1.99 | 1.90E−04 | 8.35E−04 | SPCS3 | 1.19 | 2.16E−03 | 5.16E−03 |
| NFKBIZ | 2.08 | 7.46E−06 | 1.08E−04 | RAET1L | 7.06 | 1.91E−04 | 8.38E−04 | VIMP | 1.33 | 2.16E−03 | 5.17E−03 |
| ABRACL | 1.73 | 7.53E−06 | 1.09E−04 | ELMOD1 | 4.19 | 1.93E−04 | 8.44E−04 | HTR7 | 1.29 | 2.17E−03 | 5.18E−03 |
| SYNC | 4.98 | 7.54E−06 | 1.09E−04 | BRSK1 | 1.21 | 1.94E−04 | 8.46E−04 | PODXL2 | 1.62 | 2.17E−03 | 5.19E−03 |
| TXN | 1.16 | 7.70E−06 | 1.10E−04 | CHPF2 | 1.40 | 1.94E−04 | 8.48E−04 | NOMO3 | 1.30 | 2.18E−03 | 5.21E−03 |
| FOXC1 | 2.77 | 7.79E−06 | 1.11E−04 | TTC17 | 1.37 | 1.95E−04 | 8.49E−04 | PDE4DIP | 1.35 | 2.19E−03 | 5.21E−03 |
| NFKB1 | 1.71 | 7.82E−06 | 1.11E−04 | SH3BGRL3 | 1.34 | 1.95E−04 | 8.49E−04 | PPM1A | 1.16 | 2.20E−03 | 5.23E−03 |
| FAM3C | 1.29 | 7.95E−06 | 1.13E−04 | NXPH4 | 2.53 | 1.95E−04 | 8.50E−04 | SLMAP | 1.22 | 2.25E−03 | 5.33E−03 |
| GJB1 | 1.52 | 7.95E−06 | 1.13E−04 | ATG12 | 1.36 | 1.96E−04 | 8.52E−04 | RC3H2 | 1.17 | 2.25E−03 | 5.33E−03 |
| TMEM154 | 3.26 | 7.93E−06 | 1.13E−04 | HIPK3 | 1.43 | 1.96E−04 | 8.52E−04 | ATP6V1F | 1.15 | 2.25E−03 | 5.34E−03 |
| APC | 2.11 | 8.04E−06 | 1.13E−04 | CRYL1 | 2.49 | 1.97E−04 | 8.55E−04 | ERLEC1 | 1.17 | 2.27E−03 | 5.37E−03 |
| SEZ6L2 | 1.60 | 8.02E−06 | 1.13E−04 | SLC39A13 | 1.56 | 1.97E−04 | 8.56E−04 | P3H4 | 1.13 | 2.27E−03 | 5.37E−03 |
| PLP2 | 1.38 | 8.13E−06 | 1.14E−04 | TVP23B | 1.20 | 1.98E−04 | 8.56E−04 | PFKP | 1.11 | 2.28E−03 | 5.38E−03 |
| LINC01570 | 66.53 | 8.15E−06 | 1.14E−04 | UGGT2 | 1.29 | 1.98E−04 | 8.56E−04 | LOC100129434 | 1.12 | 2.28E−03 | 5.38E−03 |
| LOC107984449 | 2.21 | 8.17E−06 | 1.14E−04 | IERS | 1.43 | 1.99E−04 | 8.60E−04 | B4GALT7 | 1.24 | 2.28E−03 | 5.40E−03 |
| PGK1 | 1.30 | 8.21E−06 | 1.14E−04 | AGA | 1.37 | 2.00E−04 | 8.61E−04 | HPRT1 | 1.32 | 2.29E−03 | 5.41E−03 |
| ARPCIA | 1.27 | 8.29E−06 | 1.15E−04 | CMTM3 | 1.42 | 1.99E−04 | 8.61E−04 | HSDL1 | 1.14 | 2.30E−03 | 5.42E−03 |
| GNG2 | 2.57 | 8.30E−06 | 1.15E−04 | TNFRSF12A | 1.36 | 1.99E−04 | 8.61E−04 | MAP3K11 | 1.18 | 2.30E−03 | 5.42E−03 |
| MAP4 | 1.17 | 8.31E−06 | 1.15E−04 | 9-Mar | 1.54 | 2.01E−04 | 8.65E−04 | SPATA20 | 1.38 | 2.30E−03 | 5.43E−03 |
| EDEM1 | 1.56 | 8.33E−06 | 1.15E−04 | WDR47 | 1.44 | 2.02E−04 | 8.67E−04 | COPE | 1.11 | 2.31E−03 | 5.44E−03 |
| NFIB | 1.93 | 8.44E−06 | 1.16E−04 | LOC101928143 | 3.10 | 2.03E−04 | 8.74E−04 | SPRYD3 | 1.46 | 2.32E−03 | 5.46E−03 |
| SLC14A1 | 4.53 | 8.60E−06 | 1.17E−04 | NOL3 | 1.38 | 2.06E−04 | 8.82E−04 | PLCB3 | 1.51 | 2.32E−03 | 5.47E−03 |
| CAPN2 | 1.30 | 8.71E−06 | 1.18E−04 | ATP6AP2 | 1.24 | 2.08E−04 | 8.89E−04 | HMGCL | 1.33 | 2.34E−03 | 5.49E−03 |
| SERPINE1 | 7.62 | 8.68E−06 | 1.18E−04 | CHST7 | 1.58 | 2.11E−04 | 8.97E−04 | TNIP2 | 1.20 | 2.34E−03 | 5.49E−03 |
| C15orf48 | 11.76 | 8.77E−06 | 1.18E−04 | GUCD1 | 1.35 | 2.13E−04 | 9.00E−04 | SOCS2 | 1.78 | 2.37E−03 | 5.56E−03 |
| ODC1 | 2.44 | 8.92E−06 | 1.20E−04 | ARHGEF1 | 1.19 | 2.13E−04 | 9.01E−04 | LINC00506 | 1.12 | 2.39E−03 | 5.60E−03 |
| AHNAK2 | 4.25 | 8.96E−06 | 1.20E−04 | TMEM9B | 1.21 | 2.14E−04 | 9.04E−04 | TBCEL | 1.15 | 2.40E−03 | 5.60E−03 |
| SEMA3A | 2.40 | 9.00E−06 | 1.20E−04 | ARHGAP22 | 3.29 | 2.15E−04 | 9.04E−04 | CD109 | 1.10 | 2.40E−03 | 5.61E−03 |
| KDM5B | 1.21 | 9.03E−06 | 1.20E−04 | MTRNR2L2 | 1.32 | 2.15E−04 | 9.05E−04 | TESK1 | 1.27 | 2.40E−03 | 5.61E−03 |
| TMX3 | 1.41 | 9.23E−06 | 1.22E−04 | DNAJB2 | 1.40 | 2.19E−04 | 9.19E−04 | ACOT8 | 1.11 | 2.41E−03 | 5.64E−03 |
| ALPK1 | 2.94 | 9.51E−06 | 1.25E−04 | DESI2 | 1.16 | 2.20E−04 | 9.21E−04 | TRIM3 | 1.35 | 2.41E−03 | 5.64E−03 |
| PIK3CD | 1.89 | 9.67E−06 | 1.27E−04 | WDTC1 | 1.26 | 2.21E−04 | 9.26E−04 | G6PC3 | 1.11 | 2.44E−03 | 5.68E−03 |
| CTSL | 1.94 | 9.73E−06 | 1.27E−04 | PPP1CB | 1.26 | 2.25E−04 | 9.35E−04 | PIP4K2B | 1.13 | 2.44E−03 | 5.68E−03 |
| CORO2B | 1.62 | 9.79E−06 | 1.27E−04 | SAR1B | 1.29 | 2.25E−04 | 9.36E−04 | YIF1A | 1.17 | 2.44E−03 | 5.69E−03 |
| MAP1LC3B | 1.53 | 9.79E−06 | 1.27E−04 | CPE | 1.12 | 2.25E−04 | 9.37E−04 | NAT6 | 1.56 | 2.45E−03 | 5.71E−03 |
| MLLT4 | 2.10 | 9.90E−06 | 1.28E−04 | SPTBN1 | 1.16 | 2.29E−04 | 9.47E−04 | TNKS | 1.15 | 2.46E−03 | 5.74E−03 |
| TMEM132A | 1.99 | 9.98E−06 | 1.29E−04 | MAML2 | 1.51 | 2.29E−04 | 9.48E−04 | PRUNE | 1.10 | 2.47E−03 | 5.75E−03 |
| IRF2BP2 | 1.70 | 1.01E−05 | 1.30E−04 | LRAT | 2.39 | 2.30E−04 | 9.49E−04 | ARSA | 1.83 | 2.53E−03 | 5.85E−03 |
| ITGB8 | 2.01 | 1.01E−05 | 1.30E−04 | CTTN | 1.22 | 2.30E−04 | 9.50E−04 | MDFIC | 1.52 | 2.53E−03 | 5.87E−03 |
| B3GNT5 | 4.34 | 1.02E−05 | 1.30E−04 | SNHG6 | 1.15 | 2.33E−04 | 9.61E−04 | SPRED2 | 1.18 | 2.54E−03 | 5.88E−03 |
| FLNA | 1.34 | 1.02E−05 | 1.30E−04 | KHNYN | 1.46 | 2.34E−04 | 9.62E−04 | LOC100507460 | 1.16 | 2.55E−03 | 5.89E−03 |
| PANX1 | 1.96 | 1.02E−05 | 1.30E−04 | MOB3C | 1.50 | 2.34E−04 | 9.63E−04 | EVIS | 1.41 | 2.56E−03 | 5.90E−03 |
| ARHGAP21 | 1.59 | 1.03E−05 | 1.30E−04 | TRPT1 | 1.51 | 2.35E−04 | 9.67E−04 | TMEM62 | 1.31 | 2.58E−03 | 5.94E−03 |
| RGS2 | 4.86 | 1.03E−05 | 1.30E−04 | KLHDC2 | 1.21 | 2.36E−04 | 9.71E−04 | MTRNR2L8 | 1.25 | 2.58E−03 | 5.94E−03 |
| SDC2 | 2.08 | 1.03E−05 | 1.30E−04 | ESRG | 1.82 | 2.38E−04 | 9.77E−04 | ATP6V1H | 1.12 | 2.58E−03 | 5.95E−03 |
| UBE2Q2 | 1.51 | 1.03E−05 | 1.30E−04 | CLU | 1.47 | 2.38E−04 | 9.78E−04 | HOMER1 | 1.10 | 2.60E−03 | 5.98E−03 |
| EFNB2 | 1.53 | 1.05E−05 | 1.31E−04 | PGM3 | 1.92 | 2.42E−04 | 9.90E−04 | C12orf49 | 1.12 | 2.62E−03 | 6.02E−03 |
| WDR45 | 1.35 | 1.05E−05 | 1.31E−04 | GAS5 | 1.33 | 2.46E−04 | 1.00E−03 | TMEM255B | 2.40 | 2.63E−03 | 6.04E−03 |
| C2CD2L | 1.37 | 1.05E−05 | 1.31E−04 | ZER1 | 1.21 | 2.46E−04 | 1.00E−03 | FAM58A | 1.09 | 2.64E−03 | 6.06E−03 |
| TOR1AIP1 | 1.18 | 1.05E−05 | 1.31E−04 | RPLPO | 1.11 | 2.50E−04 | 1.02E−03 | CAPRIN2 | 1.86 | 2.64E−03 | 6.06E−03 |
| CPEB4 | 1.69 | 1.07E−05 | 1.33E−04 | LOC729348 | 1.19 | 2.51E−04 | 1.02E−03 | CTSF | 1.20 | 2.64E−03 | 6.06E−03 |
| SRSF8 | 1.51 | 1.09E−05 | 1.35E−04 | TPP1 | 1.21 | 2.51E−04 | 1.02E−03 | BCR | 1.15 | 2.65E−03 | 6.07E−03 |
| PPARD | 3.38 | 1.09E−05 | 1.35E−04 | KLHL2 | 1.32 | 2.51E−04 | 1.02E−03 | ZADH2 | 1.27 | 2.67E−03 | 6.10E−03 |
| FMNL3 | 1.51 | 1.11E−05 | 1.36E−04 | UBALD2 | 1.59 | 2.52E−04 | 1.02E−03 | TMEM219 | 1.35 | 2.67E−03 | 6.11E−03 |
| KCNK1 | 8.77 | 1.11E−05 | 1.36E−04 | TNFRSF1A | 1.20 | 2.53E−04 | 1.02E−03 | UBR1 | 1.22 | 2.68E−03 | 6.13E−03 |
| PLXNA3 | 2.20 | 1.11E−05 | 1.36E−04 | FAT1 | 1.18 | 2.55E−04 | 1.03E−03 | PTK2 | 1.14 | 2.70E−03 | 6.16E−03 |
| TUFT1 | 1.34 | 1.11E−05 | 1.36E−04 | TMC7 | 3.12 | 2.58E−04 | 1.04E−03 | FAM127B | 1.12 | 2.71E−03 | 6.19E−03 |
| B3GAT1 | 4.19 | 1.12E−05 | 1.37E−04 | UPRT | 1.45 | 2.58E−04 | 1.04E−03 | RPS19 | 1.09 | 2.72E−03 | 6.20E−03 |
| NCS1 | 1.76 | 1.13E−05 | 1.37E−04 | LAP3 | 1.49 | 2.59E−04 | 1.04E−03 | ATP6V1D | 1.22 | 2.75E−03 | 6.26E−03 |
| STAT6 | 1.36 | 1.13E−05 | 1.38E−04 | KLC2 | 1.27 | 2.60E−04 | 1.04E−03 | ABCC10 | 1.31 | 2.76E−03 | 6.28E−03 |
| LARP6 | 1.52 | 1.14E−05 | 1.38E−04 | MANEAL | 1.45 | 2.61E−04 | 1.05E−03 | ERAP1 | 1.18 | 2.77E−03 | 6.29E−03 |
| RPS12 | 1.22 | 1.15E−05 | 1.40E−04 | LOC105369340 | 2.98 | 2.61E−04 | 1.05E−03 | FAH | 1.19 | 2.78E−03 | 6.31E−03 |
| SERPINB8 | 1.62 | 1.15E−05 | 1.40E−04 | MLLT3 | 1.87 | 2.62E−04 | 1.05E−03 | KBTBD2 | 1.18 | 2.78E−03 | 6.31E−03 |
| RELB | 2.39 | 1.16E−05 | 1.40E−04 | INPP5A | 1.55 | 2.65E−04 | 1.06E−03 | ID3 | 1.16 | 2.79E−03 | 6.33E−03 |
| TRIM8 | 1.59 | 1.16E−05 | 1.40E−04 | VAMP3 | 1.30 | 2.65E−04 | 1.06E−03 | UTRN | 1.23 | 2.80E−03 | 6.34E−03 |
| MINPP1 | 1.33 | 1.18E−05 | 1.42E−04 | MICALL1 | 1.14 | 2.67E−04 | 1.06E−03 | CAPN10 | 1.30 | 2.82E−03 | 6.39E−03 |
| PTDSS1 | 1.24 | 1.19E−05 | 1.42E−04 | RIN2 | 2.01 | 2.68E−04 | 1.07E−03 | TMEM59 | 1.19 | 2.83E−03 | 6.39E−03 |
| RIPK2 | 1.89 | 1.19E−05 | 1.42E−04 | FAM102A | 1.35 | 2.69E−04 | 1.07E−03 | TUBB4A | 1.32 | 2.83E−03 | 6.39E−03 |
| TNFRSF10B | 1.43 | 1.20E−05 | 1.43E−04 | NRBP1 | 1.20 | 2.69E−04 | 1.07E−03 | URGCP | 1.08 | 2.83E−03 | 6.39E−03 |
| ZKSCAN1 | 1.29 | 1.21E−05 | 1.43E−04 | PIGM | 1.30 | 2.69E−04 | 1.07E−03 | METTL23 | 1.16 | 2.83E−03 | 6.39E−03 |
| DENNDIA | 1.46 | 1.25E−05 | 1.47E−04 | HIST3H2A | 1.93 | 2.69E−04 | 1.07E−03 | ACOX2 | 1.29 | 2.84E−03 | 6.42E−03 |
| MACF1 | 1.28 | 1.27E−05 | 1.48E−04 | STIM2 | 1.40 | 2.69E−04 | 1.07E−03 | RAB40B | 1.84 | 2.85E−03 | 6.43E−03 |
| RPSAP52 | 6.43 | 1.27E−05 | 1.48E−04 | CLCF1 | 1.54 | 2.70E−04 | 1.07E−03 | TPD52L2 | 1.18 | 2.86E−03 | 6.46E−03 |
| LTBP4 | 1.88 | 1.28E−05 | 1.48E−04 | LOC105372580 | 1.87 | 2.70E−04 | 1.07E−03 | DOK4 | 1.40 | 2.87E−03 | 6.49E−03 |
| SPRR2D | 4.23 | 1.29E−05 | 1.49E−04 | 2-Mar | 1.25 | 2.70E−04 | 1.07E−03 | LOC105370333 | 1.06 | 2.94E−03 | 6.59E−03 |
| NET1 | 1.93 | 1.30E−05 | 1.49E−04 | CLDN12 | 1.33 | 2.71E−04 | 1.07E−03 | RAB29 | 1.12 | 2.94E−03 | 6.61E−03 |
| PLSCR3 | 1.62 | 1.30E−05 | 1.49E−04 | MSANTD3 | 1.60 | 2.72E−04 | 1.07E−03 | GALNS | 1.34 | 2.96E−03 | 6.65E−03 |
| SCYL2 | 1.24 | 1.31E−05 | 1.49E−04 | ETV3 | 1.40 | 2.72E−04 | 1.07E−03 | DEAF1 | 1.25 | 2.97E−03 | 6.65E−03 |
| CES2 | 1.27 | 1.31E−05 | 1.50E−04 | RAP1GAP2 | 1.58 | 2.73E−04 | 1.07E−03 | YWHAG | 1.09 | 2.98E−03 | 6.67E−03 |
| KDM1B | 1.60 | 1.32E−05 | 1.50E−04 | FUT8 | 1.47 | 2.74E−04 | 1.08E−03 | SELK | 1.32 | 2.98E−03 | 6.67E−03 |
| HDAC9 | 2.39 | 1.33E−05 | 1.51E−04 | MCOLN1 | 1.29 | 2.74E−04 | 1.08E−03 | JADE1 | 1.19 | 2.99E−03 | 6.67E−03 |
| RP2 | 2.05 | 1.34E−05 | 1.52E−04 | TLE1 | 1.37 | 2.74E−04 | 1.08E−03 | ARPC2 | 1.11 | 3.00E−03 | 6.69E−03 |
| CALB1 | 14.55 | 1.38E−05 | 1.55E−04 | EXOC7 | 1.24 | 2.74E−04 | 1.08E−03 | RAP1B | 1.17 | 3.00E−03 | 6.69E−03 |
| BIVM | 1.51 | 1.41E−05 | 1.57E−04 | RNA45S5 | 1.39 | 2.74E−04 | 1.08E−03 | VANGL1 | 1.11 | 3.01E−03 | 6.72E−03 |
| CARMIL2 | 3.32 | 1.42E−05 | 1.57E−04 | CCDC93 | 1.21 | 2.75E−04 | 1.08E−03 | FAM213A | 1.35 | 3.02E−03 | 6.74E−03 |
| RABAC1 | 1.90 | 1.44E−05 | 1.57E−04 | NKILA | 2.27 | 2.76E−04 | 1.08E−03 | ARNT | 1.25 | 3.03E−03 | 6.76E−03 |
| CERS2 | 1.32 | 1.45E−05 | 1.58E−04 | TXNIP | 1.69 | 2.77E−04 | 1.09E−03 | HRAS | 1.07 | 3.07E−03 | 6.84E−03 |
| MYO9B | 1.21 | 1.46E−05 | 1.58E−04 | GLTP | 1.34 | 2.78E−04 | 1.09E−03 | NT5DC3 | 1.36 | 3.13E−03 | 6.96E−03 |
| STX1A | 1.92 | 1.46E−05 | 1.58E−04 | DLX1 | 1.36 | 2.79E−04 | 1.09E−03 | TM7SF3 | 1.11 | 3.16E−03 | 7.00E−03 |
| PRKCA | 1.75 | 1.47E−05 | 1.59E−04 | FADD | 1.25 | 2.80E−04 | 1.10E−03 | TP53I13 | 1.49 | 3.17E−03 | 7.01E−03 |
| APAF1 | 1.71 | 1.49E−05 | 1.60E−04 | SBF1 | 1.24 | 2.85E−04 | 1.11E−03 | IRF1 | 1.32 | 3.17E−03 | 7.03E−03 |
| ZCCHC9 | 1.73 | 1.50E−05 | 1.61E−04 | SMAGP | 1.32 | 2.87E−04 | 1.12E−03 | NAGLU | 1.23 | 3.18E−03 | 7.03E−03 |
| MYH16 | 4.80 | 1.51E−05 | 1.61E−04 | UBA6 | 1.92 | 2.88E−04 | 1.12E−03 | MOV10 | 1.19 | 3.18E−03 | 7.05E−03 |
| DIEXF | 1.89 | 1.52E−05 | 1.62E−04 | GAGE12H | 4.22 | 2.89E−04 | 1.12E−03 | MFSD2A | 1.21 | 3.20E−03 | 7.08E−03 |
| NRIP3 | 3.24 | 1.53E−05 | 1.63E−04 | PITRM1 | 1.20 | 2.90E−04 | 1.12E−03 | CASC4 | 1.07 | 3.20E−03 | 7.08E−03 |
| TAF9B | 1.81 | 1.53E−05 | 1.63E−04 | LOC105376382 | 2.64 | 2.90E−04 | 1.13E−03 | LOC100507002 | 2.46 | 3.21E−03 | 7.10E−03 |
| SNX9 | 1.83 | 1.54E−05 | 1.63E−04 | DEFB103A | 14.24 | 2.90E−04 | 1.13E−03 | RGS16 | 1.31 | 3.22E−03 | 7.11E−03 |
| FIBP | 1.14 | 1.54E−05 | 1.64E−04 | GRIK2 | 1.33 | 2.90E−04 | 1.13E−03 | SNAP23 | 1.15 | 3.24E−03 | 7.14E−03 |
| CLSTN1 | 1.58 | 1.55E−05 | 1.64E−04 | HIPK1 | 1.24 | 2.91E−04 | 1.13E−03 | HTATIP2 | 1.18 | 3.24E−03 | 7.14E−03 |
| INPPL1 | 1.15 | 1.55E−05 | 1.64E−04 | CACYBP | 1.18 | 2.91E−04 | 1.13E−03 | MIA3 | 1.15 | 3.26E−03 | 7.17E−03 |
| CD68 | 2.20 | 1.57E−05 | 1.65E−04 | CMTM4 | 1.60 | 2.93E−04 | 1.13E−03 | ULK3 | 1.24 | 3.26E−03 | 7.17E−03 |
| NIT1 | 1.83 | 1.58E−05 | 1.66E−04 | KDSR | 1.27 | 2.93E−04 | 1.13E−03 | STARD3 | 1.13 | 3.26E−03 | 7.18E−03 |
| SERINC3 | 1.54 | 1.58E−05 | 1.66E−04 | NFE2L3 | 1.32 | 2.94E−04 | 1.14E−03 | CD276 | 1.11 | 3.26E−03 | 7.18E−03 |
| CERK | 1.34 | 1.60E−05 | 1.67E−04 | GBA | 1.26 | 2.97E−04 | 1.14E−03 | KLHL42 | 1.27 | 3.27E−03 | 7.18E−03 |
| DGKQ | 1.64 | 1.60E−05 | 1.67E−04 | TMED9 | 1.12 | 2.98E−04 | 1.15E−03 | WASL | 1.20 | 3.27E−03 | 7.18E−03 |
| ELOVL1 | 1.39 | 1.60E−05 | 1.67E−04 | OSGIN2 | 1.30 | 3.00E−04 | 1.15E−03 | CTNNA1 | 1.09 | 3.30E−03 | 7.24E−03 |
| EXT2 | 1.39 | 1.60E−05 | 1.67E−04 | SUMF1 | 1.32 | 2.99E−04 | 1.15E−03 | PHRF1 | 1.11 | 3.30E−03 | 7.25E−03 |
| FAM214B | 1.93 | 1.59E−05 | 1.67E−04 | TCP11L1 | 1.27 | 3.00E−04 | 1.15E−03 | FBXO31 | 1.09 | 3.31E−03 | 7.27E−03 |
| ZSWIM4 | 1.69 | 1.60E−05 | 1.67E−04 | GUK1 | 1.28 | 3.02E−04 | 1.16E−03 | C9orf16 | 1.21 | 3.33E−03 | 7.29E−03 |
| UAP1 | 1.69 | 1.61E−05 | 1.67E−04 | PDZD11 | 1.29 | 3.07E−04 | 1.17E−03 | CSGALNACT2 | 1.33 | 3.34E−03 | 7.31E−03 |
| ARL8A | 1.90 | 1.62E−05 | 1.67E−04 | GPAA1 | 1.14 | 3.09E−04 | 1.18E−03 | RHOG | 1.25 | 3.36E−03 | 7.35E−03 |
| RRBP1 | 1.20 | 1.63E−05 | 1.68E−04 | SLC30A9 | 1.20 | 3.11E−04 | 1.18E−03 | TRIM4 | 1.31 | 3.36E−03 | 7.35E−03 |
| ID2 | 2.58 | 1.63E−05 | 1.68E−04 | SMYD2 | 1.57 | 3.11E−04 | 1.18E−03 | POLG | 1.24 | 3.37E−03 | 7.36E−03 |
| ACAA1 | 1.76 | 1.64E−05 | 1.68E−04 | PTPN23 | 1.11 | 3.13E−04 | 1.19E−03 | CDK5RAP2 | 1.12 | 3.40E−03 | 7.41E−03 |
| PARP8 | 1.78 | 1.64E−05 | 1.68E−04 | LINC00704 | 2.81 | 3.15E−04 | 1.19E−03 | JAK1 | 1.06 | 3.40E−03 | 7.41E−03 |
| F2RL2 | 2.18 | 1.65E−05 | 1.68E−04 | YPEL3 | 1.50 | 3.16E−04 | 1.20E−03 | MSRA | 1.29 | 3.40E−03 | 7.42E−03 |
| FOXD1 | 1.56 | 1.66E−05 | 1.68E−04 | HECTD3 | 1.22 | 3.18E−04 | 1.20E−03 | ARHGDIA | 1.07 | 3.42E−03 | 7.43E−03 |
| FGFRL1 | 1.37 | 1.68E−05 | 1.70E−04 | CDC42SE2 | 1.34 | 3.20E−04 | 1.21E−03 | PPIB | 1.05 | 3.43E−03 | 7.45E−03 |
| ZDHHC24 | 1.50 | 1.70E−05 | 1.71E−04 | KIF21A | 1.48 | 3.21E−04 | 1.21E−03 | RERE | 1.08 | 3.43E−03 | 7.45E−03 |
| CCNA1 | 3.55 | 1.71E−05 | 1.72E−04 | PPP1R21 | 1.74 | 3.21E−04 | 1.21E−03 | TMEM50A | 1.24 | 3.46E−03 | 7.50E−03 |
| COL9A2 | 1.94 | 1.72E−05 | 1.72E−04 | NAPG | 1.18 | 3.26E−04 | 1.23E−03 | TOLLIP | 1.22 | 3.46E−03 | 7.50E−03 |
| FOXF1 | 3.18 | 1.72E−05 | 1.72E−04 | SLC48A1 | 1.31 | 3.30E−04 | 1.24E−03 | DBNL | 1.14 | 3.47E−03 | 7.53E−03 |
| NOTCH2 | 1.24 | 1.72E−05 | 1.72E−04 | ZSWIM6 | 1.27 | 3.30E−04 | 1.24E−03 | PLPP6 | 1.33 | 3.48E−03 | 7.54E−03 |
| PABPC1 | 1.07 | 1.72E−05 | 1.72E−04 | PJA1 | 1.80 | 3.31E−04 | 1.24E−03 | MIA | 1.16 | 3.49E−03 | 7.57E−03 |
| CXCL3 | 10.57 | 1.74E−05 | 1.73E−04 | USP12 | 1.34 | 3.34E−04 | 1.25E−03 | IKBIP | 1.16 | 3.49E−03 | 7.57E−03 |
| HIST2H2BE | 2.90 | 1.74E−05 | 1.73E−04 | ARSD | 1.59 | 3.37E−04 | 1.26E−03 | PART1 | 1.58 | 3.50E−03 | 7.58E−03 |
| ALKBH8 | 1.51 | 1.75E−05 | 1.74E−04 | AQP3 | 2.19 | 3.38E−04 | 1.26E−03 | CD63 | 1.06 | 3.53E−03 | 7.63E−03 |
| GAS2L1 | 1.98 | 1.75E−05 | 1.74E−04 | GRAMD1A | 1.35 | 3.38E−04 | 1.26E−03 | ZBTB7A | 1.22 | 3.54E−03 | 7.63E−03 |
| SWAP70 | 1.46 | 1.75E−05 | 1.74E−04 | MLLT11 | 1.29 | 3.41E−04 | 1.27E−03 | TGIF1 | 1.26 | 3.54E−03 | 7.64E−03 |
| ELL2 | 2.99 | 1.78E−05 | 1.75E−04 | ITGAV | 1.29 | 3.44E−04 | 1.28E−03 | KIDINS220 | 1.14 | 3.57E−03 | 7.68E−03 |
| SOX4 | 2.17 | 1.80E−05 | 1.76E−04 | PTBP3 | 1.24 | 3.45E−04 | 1.28E−03 | TIMM10B | 1.15 | 3.58E−03 | 7.70E−03 |
| ECM1 | 1.82 | 1.81E−05 | 1.77E−04 | PACS1 | 1.24 | 3.45E−04 | 1.28E−03 | BCORL1 | 1.43 | 3.61E−03 | 7.75E−03 |
| GDI1 | 1.67 | 1.81E−05 | 1.77E−04 | UPP1 | 1.51 | 3.47E−04 | 1.29E−03 | HERC4 | 1.22 | 3.61E−03 | 7.75E−03 |
| AGAP2-AS1 | 1.68 | 1.81E−05 | 1.77E−04 | EIF2S3 | 1.11 | 3.51E−04 | 1.30E−03 | AKIRIN2 | 1.17 | 3.61E−03 | 7.75E−03 |
| SLC22A18 | 1.38 | 1.82E−05 | 1.77E−04 | KIAA0754 | 1.25 | 3.52E−04 | 1.30E−03 | GLB1 | 1.11 | 3.63E−03 | 7.78E−03 |
| ATP2B4 | 1.45 | 1.83E−05 | 1.78E−04 | DHRS7 | 1.98 | 3.56E−04 | 1.31E−03 | IGF1R | 1.09 | 3.63E−03 | 7.79E−03 |
| RASA1 | 2.47 | 1.83E−05 | 1.78E−04 | NFIL3 | 1.36 | 3.60E−04 | 1.33E−03 | LOC283788 | 1.25 | 3.67E−03 | 7.86E−03 |
| NUMB | 1.44 | 1.84E−05 | 1.78E−04 | PLGRKT | 1.45 | 3.60E−04 | 1.33E−03 | MFSD10 | 1.13 | 3.67E−03 | 7.86E−03 |
| CLASP1 | 1.53 | 1.88E−05 | 1.80E−04 | TPMT | 1.42 | 3.62E−04 | 1.33E−03 | PLD2 | 1.22 | 3.70E−03 | 7.91E−03 |
| TMEM121 | 1.70 | 1.88E−05 | 1.80E−04 | SLC35E1 | 1.16 | 3.64E−04 | 1.34E−03 | ZSWIM8 | 1.12 | 3.71E−03 | 7.92E−03 |
| ADGRG6 | 3.19 | 1.89E−05 | 1.81E−04 | SIPA1L1 | 1.84 | 3.65E−04 | 1.34E−03 | PDCD10 | 1.17 | 3.74E−03 | 7.98E−03 |
| ANTXR2 | 2.69 | 1.92E−05 | 1.83E−04 | EIF4EBP2 | 1.13 | 3.67E−04 | 1.35E−03 | FAM177A1 | 1.44 | 3.74E−03 | 7.98E−03 |
| MLC1 | 10.91 | 1.92E−05 | 1.83E−04 | GPR107 | 1.23 | 3.72E−04 | 1.36E−03 | STK4 | 1.16 | 3.77E−03 | 8.02E−03 |
| NFKBIA | 2.87 | 1.97E−05 | 1.87E−04 | ENO2 | 1.42 | 3.72E−04 | 1.36E−03 | STX7 | 1.32 | 3.79E−03 | 8.06E−03 |
| BTC | 9.15 | 1.99E−05 | 1.87E−04 | CMTM6 | 1.14 | 3.72E−04 | 1.36E−03 | FBXO17 | 1.21 | 3.84E−03 | 8.15E−03 |
| ITPR3 | 1.54 | 1.99E−05 | 1.87E−04 | SAMD9 | 1.80 | 3.72E−04 | 1.36E−03 | EVI5L | 1.25 | 3.85E−03 | 8.17E−03 |
| CST3 | 1.69 | 2.00E−05 | 1.87E−04 | GNG10 | 1.23 | 3.73E−04 | 1.36E−03 | SERTAD1 | 1.46 | 3.87E−03 | 8.20E−03 |
| CAPS | 1.55 | 2.01E−05 | 1.88E−04 | CAST | 1.20 | 3.73E−04 | 1.36E−03 | ERBIN | 1.16 | 3.87E−03 | 8.21E−03 |
| CD99 | 1.29 | 2.02E−05 | 1.89E−04 | P4HA2 | 1.58 | 3.74E−04 | 1.36E−03 | CHCHD2 | 1.09 | 3.89E−03 | 8.23E−03 |
| EEF1A2 | 1.49 | 2.05E−05 | 1.91E−04 | SEMA7A | 1.17 | 3.77E−04 | 1.37E−03 | ERP44 | 1.20 | 3.91E−03 | 8.28E−03 |
| LAMC2 | 7.02 | 2.06E−05 | 1.91E−04 | ATL3 | 1.40 | 3.81E−04 | 1.38E−03 | PPIC | 1.29 | 3.92E−03 | 8.29E−03 |
| CFL1 | 1.15 | 2.08E−05 | 1.92E−04 | IRF9 | 3.03 | 3.83E−04 | 1.39E−03 | NUDCD3 | 1.11 | 3.93E−03 | 8.32E−03 |
| IGSF9B | 3.19 | 2.09E−05 | 1.93E−04 | OPHN1 | 1.32 | 3.83E−04 | 1.39E−03 | ATP6V0E1 | 1.19 | 3.94E−03 | 8.33E−03 |
| EFNB1 | 2.33 | 2.10E−05 | 1.94E−04 | ABTB1 | 1.86 | 3.87E−04 | 1.40E−03 | AMACR | 1.26 | 3.97E−03 | 8.38E−03 |
| EVC | 2.54 | 2.11E−05 | 1.94E−04 | CYB5R3 | 1.15 | 3.87E−04 | 1.40E−03 | ICA1 | 1.45 | 3.99E−03 | 8.41E−03 |
| DVL1 | 1.68 | 2.12E−05 | 1.94E−04 | LRP1 | 1.36 | 3.88E−04 | 1.40E−03 | CDKN1B | 1.14 | 3.99E−03 | 8.42E−03 |
| EPB41L3 | 13.86 | 2.13E−05 | 1.95E−04 | SEC14L2 | 1.46 | 3.88E−04 | 1.40E−03 | ESRRA | 1.16 | 4.00E−03 | 8.43E−03 |
| GJB3 | 2.74 | 2.12E−05 | 1.95E−04 | HOMER2 | 2.08 | 3.90E−04 | 1.40E−03 | TMEM14A | 1.44 | 4.01E−03 | 8.43E−03 |
| AKR1C1 | 2.20 | 2.14E−05 | 1.95E−04 | AP3S1 | 1.33 | 3.90E−04 | 1.40E−03 | IGBP1 | 1.19 | 4.01E−03 | 8.43E−03 |
| RPL3 | 1.14 | 2.16E−05 | 1.97E−04 | BAG1 | 1.13 | 3.91E−04 | 1.41E−03 | PCNX3 | 1.19 | 4.02E−03 | 8.45E−03 |
| CAPG | 1.34 | 2.18E−05 | 1.98E−04 | SLC35G2 | 1.48 | 3.95E−04 | 1.42E−03 | NRAS | 1.04 | 4.03E−03 | 8.47E−03 |
| STK17B | 2.10 | 2.19E−05 | 1.99E−04 | CYTB | 1.29 | 3.98E−04 | 1.43E−03 | INPP5K | 1.17 | 4.04E−03 | 8.49E−03 |
| FRMD4B | 2.02 | 2.20E−05 | 2.00E−04 | CYBRD1 | 1.33 | 3.99E−04 | 1.43E−03 | KAT14 | 1.21 | 4.04E−03 | 8.49E−03 |
| ARRDC1 | 1.68 | 2.22E−05 | 2.01E−04 | DNAJC3 | 1.18 | 4.04E−04 | 1.45E−03 | KDELR1 | 1.16 | 4.06E−03 | 8.52E−03 |
| ACSL1 | 1.98 | 2.24E−05 | 2.02E−04 | TMED4 | 1.21 | 4.06E−04 | 1.45E−03 | MAPK8IP1 | 1.25 | 4.10E−03 | 8.59E−03 |
| FZD8 | 1.65 | 2.24E−05 | 2.02E−04 | TNK2 | 1.17 | 4.07E−04 | 1.45E−03 | C1orf122 | 1.23 | 4.10E−03 | 8.59E−03 |
| CHPF | 1.61 | 2.25E−05 | 2.02E−04 | DPY19L1 | 1.17 | 4.09E−04 | 1.46E−03 | GHDC | 1.22 | 4.10E−03 | 8.59E−03 |
| IRGQ | 1.66 | 2.26E−05 | 2.03E−04 | FLJ20021 | 2.15 | 4.09E−04 | 1.46E−03 | MORC3 | 1.22 | 4.11E−03 | 8.60E−03 |
| TMEM117 | 2.91 | 2.27E−05 | 2.04E−04 | SSR4 | 1.36 | 4.09E−04 | 1.46E−03 | NIN | 1.17 | 4.11E−03 | 8.60E−03 |
| PRB1 | 12.87 | 2.29E−05 | 2.05E−04 | MFGE8 | 1.56 | 4.10E−04 | 1.46E−03 | TRAM1 | 1.08 | 4.14E−03 | 8.66E−03 |
| TNFAIP3 | 2.11 | 2.30E−05 | 2.05E−04 | SDSL | 1.65 | 4.13E−04 | 1.47E−03 | LRRC75A-AS1 | 1.12 | 4.15E−03 | 8.67E−03 |
| DUSP10 | 1.66 | 2.31E−05 | 2.05E−04 | ARF1 | 1.09 | 4.16E−04 | 1.48E−03 | GNB4 | 1.28 | 4.15E−03 | 8.67E−03 |
| CUX1 | 1.42 | 2.32E−05 | 2.06E−04 | FOXC2 | 2.17 | 4.16E−04 | 1.48E−03 | CREB3 | 1.24 | 4.16E−03 | 8.69E−03 |
| OSBPL8 | 1.33 | 2.33E−05 | 2.06E−04 | ECE1 | 1.12 | 4.19E−04 | 1.48E−03 | EIF3F | 1.08 | 4.16E−03 | 8.69E−03 |
| KLF13 | 1.97 | 2.34E−05 | 2.07E−04 | YPEL5 | 1.51 | 4.20E−04 | 1.49E−03 | TBC1D8 | 1.16 | 4.16E−03 | 8.69E−03 |
| LOC79160 | 3.78 | 2.34E−05 | 2.07E−04 | TANGO2 | 1.51 | 4.23E−04 | 1.50E−03 | LOC107984717 | 1.13 | 4.19E−03 | 8.72E−03 |
| RPLP1 | 1.17 | 2.34E−05 | 2.07E−04 | RASA2 | 1.72 | 4.28E−04 | 1.51E−03 | TDRKH | 1.24 | 4.21E−03 | 8.77E−03 |
| EYA3 | 1.37 | 2.38E−05 | 2.09E−04 | UNC13B | 1.63 | 4.29E−04 | 1.51E−03 | ZNF252P | 1.19 | 4.22E−03 | 8.78E−03 |
| VLDLR | 2.35 | 2.37E−05 | 2.09E−04 | PLIN3 | 1.20 | 4.30E−04 | 1.51E−03 | ASAP2 | 1.09 | 4.24E−03 | 8.81E−03 |
| IER2 | 1.38 | 2.38E−05 | 2.09E−04 | LARP1 | 1.11 | 4.38E−04 | 1.54E−03 | GYG1 | 1.12 | 4.25E−03 | 8.83E−03 |
| RUNX1 | 1.84 | 2.40E−05 | 2.10E−04 | RPL13A | 1.09 | 4.42E−04 | 1.55E−03 | TMCO4 | 1.26 | 4.25E−03 | 8.84E−03 |
| EBLN3 | 1.28 | 2.41E−05 | 2.10E−04 | LRP8 | 1.23 | 4.42E−04 | 1.55E−03 | RNF146 | 1.23 | 4.26E−03 | 8.84E−03 |
| TGFBR1 | 1.63 | 2.41E−05 | 2.10E−04 | SPATA13 | 1.30 | 4.43E−04 | 1.55E−03 | TPRG1L | 1.38 | 4.27E−03 | 8.86E−03 |
| BCAP31 | 1.32 | 2.42E−05 | 2.10E−04 | TMC01 | 1.16 | 4.46E−04 | 1.56E−03 | PRMT2 | 1.17 | 4.28E−03 | 8.88E−03 |
| AJAP1 | 2.80 | 2.43E−05 | 2.11E−04 | DOCK5 | 1.43 | 4.48E−04 | 1.57E−03 | JRKL | 1.27 | 4.29E−03 | 8.89E−03 |
| STK17A | 1.97 | 2.43E−05 | 2.11E−04 | TAF13 | 1.28 | 4.49E−04 | 1.57E−03 | AKT1 | 1.17 | 4.31E−03 | 8.92E−03 |
| AHCYL2 | 1.89 | 2.45E−05 | 2.13E−04 | PML | 1.34 | 4.50E−04 | 1.57E−03 | RNF4 | 1.13 | 4.32E−03 | 8.93E−03 |
| IL1RL1 | 3.62 | 2.50E−05 | 2.15E−04 | LRPAP1 | 1.14 | 4.51E−04 | 1.57E−03 | IKBKB | 1.18 | 4.35E−03 | 8.98E−03 |
| JARID2 | 1.90 | 2.53E−05 | 2.17E−04 | DNTTIP1 | 1.30 | 4.51E−04 | 1.57E−03 | DCAF12 | 1.11 | 4.37E−03 | 9.02E−03 |
| PCBP4 | 1.31 | 2.53E−05 | 2.17E−04 | TRIM56 | 1.24 | 4.56E−04 | 1.59E−03 | MTRNR2L1 | 1.21 | 4.38E−03 | 9.04E−03 |
| SFT2D2 | 1.86 | 2.55E−05 | 2.18E−04 | EEF1G | 1.05 | 4.59E−04 | 1.59E−03 | CDC42 | 1.10 | 4.39E−03 | 9.05E−03 |
| F11R | 1.95 | 2.58E−05 | 2.21E−04 | SUCO | 1.25 | 4.61E−04 | 1.60E−03 | HIPK2 | 1.12 | 4.39E−03 | 9.05E−03 |
| DSC2 | 5.72 | 2.64E−05 | 2.24E−04 | TICAM1 | 1.58 | 4.63E−04 | 1.61E−03 | ATP6V0C | 1.15 | 4.40E−03 | 9.07E−03 |
| USP36 | 1.27 | 2.64E−05 | 2.24E−04 | C11orf24 | 1.12 | 4.65E−04 | 1.61E−03 | AMPD2 | 1.13 | 4.41E−03 | 9.08E−03 |
| SLC6A15 | 1.43 | 2.65E−05 | 2.24E−04 | PIGT | 1.14 | 4.66E−04 | 1.62E−03 | INF2 | 1.18 | 4.42E−03 | 9.10E−03 |
| PPP3CA | 1.99 | 2.67E−05 | 2.26E−04 | TUG1 | 1.20 | 4.69E−04 | 1.62E−03 | CTTNBP2NL | 1.33 | 4.43E−03 | 9.12E−03 |
| RNPEPL1 | 2.58 | 2.68E−05 | 2.26E−04 | LOC105371453 | 1.25 | 4.72E−04 | 1.63E−03 | LCMT2 | 1.16 | 4.45E−03 | 9.14E−03 |
| C12orf75 | 1.86 | 2.71E−05 | 2.28E−04 | CLDN4 | 1.52 | 4.72E−04 | 1.63E−03 | EXOC4 | 1.09 | 4.47E−03 | 9.18E−03 |
| OSTM1 | 1.37 | 2.73E−05 | 2.29E−04 | PSEN1 | 1.44 | 4.72E−04 | 1.63E−03 | PRDX2 | 1.11 | 4.48E−03 | 9.19E−03 |
| ABCA2 | 1.45 | 2.76E−05 | 2.30E−04 | TGFB1I1 | 1.72 | 4.73E−04 | 1.63E−03 | LOC101927374 | 1.30 | 4.50E−03 | 9.23E−03 |
| ACTN4 | 1.19 | 2.85E−05 | 2.37E−04 | ACTA2 | 1.59 | 4.77E−04 | 1.64E−03 | CTBP1-AS2 | 1.11 | 4.51E−03 | 9.23E−03 |
| HOMER3 | 1.66 | 2.88E−05 | 2.39E−04 | EFCAB14 | 1.30 | 4.76E−04 | 1.64E−03 | HIST2H4A | 1.51 | 4.54E−03 | 9.28E−03 |
| TGFB1 | 1.62 | 2.92E−05 | 2.41E−04 | PCYOX1 | 1.07 | 4.78E−04 | 1.64E−03 | HIST2H4B | 1.51 | 4.54E−03 | 9.28E−03 |
| MOAP1 | 1.30 | 2.94E−05 | 2.42E−04 | PHLDB1 | 1.29 | 4.86E−04 | 1.67E−03 | YIPF2 | 1.10 | 4.55E−03 | 9.30E−03 |
| SLC24A3 | 1.58 | 2.94E−05 | 2.42E−04 | TRHDE-AS1 | 1.53 | 4.87E−04 | 1.67E−03 | EPM2AIP1 | 1.12 | 4.56E−03 | 9.32E−03 |
| SP3 | 1.50 | 2.95E−05 | 2.42E−04 | MEIS3 | 2.11 | 4.90E−04 | 1.68E−03 | MED15 | 1.15 | 4.57E−03 | 9.32E−03 |
| AP1S2 | 1.55 | 2.98E−05 | 2.43E−04 | PTPN9 | 1.20 | 4.92E−04 | 1.68E−03 | RPL28 | 1.07 | 4.57E−03 | 9.32E−03 |
| TCN2 | 3.18 | 3.00E−05 | 2.45E−04 | EMP1 | 1.25 | 4.92E−04 | 1.68E−03 | SEMA3B | 1.12 | 4.57E−03 | 9.32E−03 |
| SLC9A3R2 | 1.65 | 3.01E−05 | 2.45E−04 | PCOLCE2 | 2.27 | 4.95E−04 | 1.69E−03 | RFWD2 | 1.22 | 4.59E−03 | 9.36E−03 |
| SLC25A30 | 1.66 | 3.06E−05 | 2.48E−04 | CRLF1 | 2.92 | 4.96E−04 | 1.69E−03 | ZNF697 | 1.20 | 4.59E−03 | 9.36E−03 |
| SHC4 | 1.92 | 3.08E−05 | 2.50E−04 | SPHK1 | 1.55 | 4.96E−04 | 1.69E−03 | FLYWCH1 | 1.15 | 4.60E−03 | 9.36E−03 |
| SMPD1 | 2.09 | 3.11E−05 | 2.51E−04 | NEK7 | 1.17 | 4.99E−04 | 1.70E−03 | SEC23B | 1.09 | 4.60E−03 | 9.37E−03 |
| CEND1 | 1.95 | 3.11E−05 | 2.51E−04 | PINK1 | 1.74 | 4.99E−04 | 1.70E−03 | ACVR1 | 1.13 | 4.60E−03 | 9.37E−03 |
| CDH6 | 12.48 | 3.12E−05 | 2.52E−04 | LYPLA2 | 1.16 | 5.01E−04 | 1.70E−03 | CMIP | 1.20 | 4.61E−03 | 9.38E−03 |
| EMP3 | 1.34 | 3.13E−05 | 2.52E−04 | ATP6AP1 | 1.17 | 5.03E−04 | 1.71E−03 | HELZ2 | 2.58 | 4.63E−03 | 9.42E−03 |
| SPSB4 | 3.58 | 3.15E−05 | 2.53E−04 | RPL39 | 1.09 | 5.03E−04 | 1.71E−03 | PCSK7 | 1.15 | 4.65E−03 | 9.44E−03 |
| APBB2 | 1.78 | 3.15E−05 | 2.53E−04 | UGDH-AS1 | 1.20 | 5.04E−04 | 1.71E−03 | MAST3 | 1.19 | 4.68E−03 | 9.49E−03 |
| PSMD2 | 1.35 | 3.17E−05 | 2.54E−04 | FLII | 1.12 | 5.06E−04 | 1.71E−03 | FANCE | 1.32 | 4.70E−03 | 9.52E−03 |
| CD151 | 1.31 | 3.19E−05 | 2.54E−04 | FOXK1 | 1.31 | 5.08E−04 | 1.72E−03 | MCFD2 | 1.10 | 4.72E−03 | 9.56E−03 |
| PRDM2 | 1.65 | 3.20E−05 | 2.55E−04 | CANT1 | 1.28 | 5.10E−04 | 1.72E−03 | KDM5C | 1.13 | 4.73E−03 | 9.58E−03 |
| ROBO4 | 2.04 | 3.21E−05 | 2.55E−04 | TCIRG1 | 1.45 | 5.12E−04 | 1.73E−03 | RMND5B | 1.27 | 4.74E−03 | 9.58E−03 |
| STXBP1 | 1.44 | 3.24E−05 | 2.56E−04 | CDS2 | 1.31 | 5.13E−04 | 1.73E−03 | C4orf48 | 1.15 | 4.75E−03 | 9.59E−03 |
| TMCO3 | 1.38 | 3.28E−05 | 2.58E−04 | CTSA | 1.23 | 5.16E−04 | 1.73E−03 | MC1R | 1.21 | 4.76E−03 | 9.61E−03 |
| C3orf52 | 2.64 | 3.30E−05 | 2.60E−04 | FBRS | 1.10 | 5.16E−04 | 1.73E−03 | AP5Z1 | 1.15 | 4.78E−03 | 9.65E−03 |
| APLP2 | 1.56 | 3.38E−05 | 2.64E−04 | CPPED1 | 1.30 | 5.18E−04 | 1.74E−03 | COMMD6 | 1.21 | 4.79E−03 | 9.67E−03 |
| TMED7 | 1.14 | 3.39E−05 | 2.64E−04 | YRDC | 1.14 | 5.23E−04 | 1.75E−03 | HEXA | 1.17 | 4.82E−03 | 9.72E−03 |
| IL37 | 7.41 | 3.45E−05 | 2.67E−04 | TMUB2 | 1.27 | 5.29E−04 | 1.77E−03 | LOC652276 | 1.29 | 4.82E−03 | 9.72E−03 |
| MIR4697HG | 2.74 | 3.48E−05 | 2.68E−04 | APMAP | 1.22 | 5.36E−04 | 1.79E−03 | PARP10 | 1.38 | 4.83E−03 | 9.73E−03 |
| SEC24D | 1.67 | 3.51E−05 | 2.69E−04 | TP53BP2 | 1.17 | 5.39E−04 | 1.79E−03 | ENOX2 | 1.24 | 4.84E−03 | 9.74E−03 |
| CSNK1E | 1.29 | 3.52E−05 | 2.70E−04 | H3F3B | 1.14 | 5.41E−04 | 1.80E−03 | AGPAT3 | 1.17 | 4.85E−03 | 9.77E−03 |
| TDRD7 | 2.15 | 3.53E−05 | 2.70E−04 | RAB38 | 1.30 | 5.42E−04 | 1.80E−03 | ELFN1-AS1 | 1.22 | 4.86E−03 | 9.77E−03 |
| FRMD8 | 1.38 | 3.56E−05 | 2.71E−04 | WASF3 | 1.30 | 5.43E−04 | 1.80E−03 | FAM96B | 1.07 | 4.86E−03 | 9.78E−03 |
| ASB1 | 1.54 | 3.57E−05 | 2.71E−04 | AGFG1 | 1.24 | 5.45E−04 | 1.81E−03 | TRIM32 | 1.12 | 4.88E−03 | 9.82E−03 |
| FAM83G | 1.46 | 3.58E−05 | 2.72E−04 | MPP6 | 1.20 | 5.45E−04 | 1.81E−03 | STK25 | 1.04 | 4.89E−03 | 9.83E−03 |
| LINC00707 | 1.96 | 3.61E−05 | 2.72E−04 | NUP50-AS1 | 2.34 | 5.51E−04 | 1.82E−03 | INO80C | 1.41 | 4.91E−03 | 9.87E−03 |
| SNX25 | 1.70 | 3.62E−05 | 2.73E−04 | NCEH1 | 1.19 | 5.52E−04 | 1.83E−03 | DRAM1 | 1.16 | 4.92E−03 | 9.87E−03 |
| APPL2 | 1.52 | 3.64E−05 | 2.74E−04 | STX16 | 1.09 | 5.53E−04 | 1.83E−03 | GOLGA1 | 1.19 | 4.92E−03 | 9.88E−03 |
| KCNMA1 | 5.17 | 3.65E−05 | 2.74E−04 | SKIL | 1.46 | 5.55E−04 | 1.83E−03 | FKBP2 | 1.30 | 4.93E−03 | 9.89E−03 |
| XYLT1 | 5.93 | 3.73E−05 | 2.79E−04 | PURB | 1.14 | 5.59E−04 | 1.84E−03 | CCPG1 | 1.15 | 4.96E−03 | 9.94E−03 |
| NR2F2 | 1.51 | 3.75E−05 | 2.80E−04 | ITPKC | 1.22 | 5.60E−04 | 1.84E−03 | RAP1GDS1 | 1.15 | 4.97E−03 | 9.95E−03 |

CD274 Genes

| Isoform | Fold | Pval | Padj |
|---|---|---|---|
| ALKBH8 | 1.15 | 5.75E−06 | 5.57E−03 |
| ANGPTL2 | 1.51 | 2.71E−05 | 8.81E−03 |
| CD274 | 1773.14 | 7.55E−07 | 2.20E−03 |
| CSGALNACT1 | 1.30 | 1.54E−05 | 8.24E−03 |
| FDCSP | 2.70 | 3.36E−05 | 9.83E−03 |
| LRRC45 | 1.10 | 2.65E−06 | 5.29E−03 |
| PMEPA1 | 1.55 | 6.05E−07 | 2.20E−03 |
| PXYLP1 | 1.25 | 2.56E−05 | 8.81E−03 |
| TMX4 | 1.26 | 1.89E−05 | 8.24E−03 |

B3GNT2 Genes

| Isoform | Fold | Pval | Padj |
|---|---|---|---|
| ADAM19 | 2.01 | 1.10E−06 | 2.15E−03 |
| ATOX1 | 1.45 | 2.38E−06 | 2.15E−03 |
| B3GNT2 | 215.39 | 5.62E−07 | 2.15E−03 |
| DNAJC3 | 1.38 | 1.84E−06 | 2.15E−03 |
| DUSP10 | 1.65 | 1.53E−06 | 2.15E−03 |
| GPNMB | 1.98 | 2.18E−06 | 2.15E−03 |
| IL24 | 2.89 | 3.44E−06 | 2.15E−03 |
| SQSTM1 | 2.25 | 8.26E−07 | 2.15E−03 |
| ST3GAL4 | 1.26 | 3.06E−06 | 2.15E−03 |
| TGFBI | 1.50 | 2.50E−06 | 2.15E−03 |
| TTC17 | 1.58 | 1.95E−06 | 2.15E−03 |
| TXNRD1 | 1.41 | 3.29E−06 | 2.15E−03 |
| CANX | 1.28 | 6.07E−06 | 2.95E−03 |
| P4HB | 1.20 | 5.44E−06 | 2.95E−03 |
| DUSP4 | 1.31 | 1.27E−05 | 3.54E−03 |
| KANK1 | 1.33 | 1.04E−05 | 3.54E−03 |
| NRP2 | 1.49 | 1.08E−05 | 3.54E−03 |
| PMEPA1 | 1.28 | 9.37E−06 | 3.54E−03 |
| CALR | 1.23 | 1.44E−05 | 3.76E−03 |
| FTH1 | 1.43 | 1.52E−05 | 3.76E−03 |
| LRRC59 | 1.14 | 1.59E−05 | 3.76E−03 |
| SCD | 1.22 | 2.19E−05 | 4.45E−03 |
| RHOQ | 1.27 | 2.43E−05 | 4.72E−03 |
| SLC37A2 | 1.45 | 2.58E−05 | 4.79E−03 |
| ATP6V0B | 1.25 | 3.11E−05 | 4.98E−03 |
| ATP6V1E1 | 1.22 | 3.01E−05 | 4.98E−03 |
| LIF | 1.51 | 2.86E−05 | 4.98E−03 |
| NGFR | 1.37 | 3.27E−05 | 4.98E−03 |
| TKT | 1.13 | 3.30E−05 | 4.98E−03 |
| ARHGEF4 | 1.90 | 4.00E−05 | 5.37E−03 |
| FGFR1 | 1.31 | 3.77E−05 | 5.37E−03 |
| PKM | 1.06 | 3.75E−05 | 5.37E−03 |
| FKBP1A | 1.12 | 4.26E−05 | 5.65E−03 |
| INSIG1 | 1.73 | 4.36E−05 | 5.68E−03 |
| GSR | 1.16 | 4.74E−05 | 6.00E−03 |
| CXCL8 | 1.71 | 5.07E−05 | 6.15E−03 |
| KYNU | 1.52 | 5.01E−05 | 6.15E−03 |
| CD63 | 1.19 | 5.56E−05 | 6.39E−03 |
| GJB1 | 1.69 | 5.86E−05 | 6.41E−03 |
| MAP1B | 1.24 | 5.77E−05 | 6.41E−03 |
| WNT9A | 2.11 | 5.87E−05 | 6.41E−03 |
| FERMT1 | 1.33 | 5.95E−05 | 6.42E−03 |
| CITED1 | 3.93 | 6.17E−05 | 6.49E−03 |
| RPN1 | 1.13 | 6.16E−05 | 6.49E−03 |
| PLIN2 | 1.21 | 6.66E−05 | 6.69E−03 |
| ATP6V0A4 | 2.46 | 7.56E−05 | 7.04E−03 |
| MCF2L | 1.39 | 8.47E−05 | 7.04E−03 |
| PHLDA1 | 1.22 | 8.24E−05 | 7.04E−03 |
| SEC61A1 | 1.17 | 8.44E−05 | 7.04E−03 |
| SURF4 | 1.18 | 7.83E−05 | 7.04E−03 |
| TFRC | 1.28 | 7.86E−05 | 7.04E−03 |
| UAP1L1 | 2.03 | 7.72E−05 | 7.04E−03 |
| MGAT4B | 1.42 | 9.24E−05 | 7.41E−03 |
| NEFL | 1.75 | 9.74E−05 | 7.47E−03 |
| CAPN1 | 1.10 | 9.97E−05 | 7.51E−03 |
| ALDOA | 1.10 | 1.01E−04 | 7.52E−03 |
| C3 | 1.59 | 1.05E−04 | 7.69E−03 |
| ASAH1 | 1.41 | 1.07E−04 | 7.76E−03 |
| SLC7A11 | 1.66 | 1.08E−04 | 7.81E−03 |
| EPT1 | 1.12 | 1.19E−04 | 8.25E−03 |
| OAF | 1.14 | 1.18E−04 | 8.25E−03 |
| TBC1D14 | 1.24 | 1.19E−04 | 8.25E−03 |
| SND1 | 1.10 | 1.20E−04 | 8.27E−03 |
| MLEC | 1.17 | 1.25E−04 | 8.38E−03 |
| CHST11 | 1.48 | 1.27E−04 | 8.41E−03 |
| PDIA4 | 1.22 | 1.29E−04 | 8.46E−03 |
| FTL | 1.19 | 1.32E−04 | 8.51E−03 |
| SAT2 | 1.21 | 1.34E−04 | 8.54E−03 |
| EDEM1 | 1.38 | 1.36E−04 | 8.56E−03 |
| FDCSP | 2.17 | 1.37E−04 | 8.56E−03 |
| MPI | 1.12 | 1.37E−04 | 8.56E−03 |
| ITPR3 | 1.35 | 1.41E−04 | 8.76E−03 |
| APOBEC3G | 1.80 | 1.44E−04 | 8.78E−03 |
| ENO2 | 1.52 | 1.63E−04 | 8.93E−03 |
| FBXO27 | 1.49 | 1.63E−04 | 8.93E−03 |
| HSPA5 | 1.16 | 1.60E−04 | 8.93E−03 |
| LMNA | 1.08 | 1.54E−04 | 8.93E−03 |
| MANF | 1.23 | 1.56E−04 | 8.93E−03 |
| MGST1 | 1.35 | 1.51E−04 | 8.93E−03 |
| NOV | 2.55 | 1.54E−04 | 8.93E−03 |
| DPY19L1 | 1.17 | 1.67E−04 | 8.94E−03 |
| MMP14 | 1.19 | 1.69E−04 | 8.96E−03 |
| NEU1 | 1.22 | 1.72E−04 | 9.04E−03 |
| CREB3L2 | 1.35 | 1.77E−04 | 9.16E−03 |
| SLC7A5 | 1.17 | 1.78E−04 | 9.16E−03 |
| PDIA6 | 1.20 | 1.88E−04 | 9.19E−03 |
| TPP1 | 1.15 | 1.93E−04 | 9.35E−03 |
| RGS16 | 1.57 | 2.08E−04 | 9.84E−03 |
| USP12 | 1.13 | 2.07E−04 | 9.84E−03 |
| G6PD | 1.15 | 2.13E−04 | 9.96E−03 |
| HS1BP3 | 1.57 | 2.14E−04 | 9.96E−03 |

MCL1 Genes

| Isoform | Fold | Pval | Padj |
|---|---|---|---|
| MCL1 | 8.65 | 5.89E−10 | 2.58E−06 |
| TIMP1 | 1.72 | 2.96E−10 | 2.58E−06 |
| HLA-B | 1.54 | 1.73E−07 | 3.02E−04 |
| HLA-C | 1.40 | 2.19E−07 | 3.02E−04 |
| RAMP1 | 1.60 | 2.34E−07 | 3.02E−04 |
| CD74 | 1.25 | 4.14E−07 | 4.03E−04 |
| GRN | 1.27 | 4.99E−07 | 4.37E−04 |
| LTBP4 | 1.15 | 2.27E−06 | 1.25E−03 |
| SOD3 | 2.52 | 2.17E−06 | 1.25E−03 |
| PMEPA1 | 1.33 | 3.68E−06 | 1.58E−03 |
| IRS2 | 1.14 | 5.42E−06 | 1.91E−03 |
| ITGB3 | 1.19 | 6.09E−06 | 1.91E−03 |
| SDC2 | 1.55 | 5.77E−06 | 1.91E−03 |
| S100A1 | 1.77 | 6.55E−06 | 1.98E−03 |
| HAPLN1 | 1.69 | 9.96E−06 | 2.50E−03 |
| S100A13 | 1.22 | 9.85E−06 | 2.50E−03 |
| SCAND1 | 1.25 | 1.02E−05 | 2.50E−03 |
| INHBA | 1.60 | 1.07E−05 | 2.53E−03 |
| DHCR24 | 1.17 | 1.17E−05 | 2.54E−03 |
| GALNT2 | 1.19 | 1.15E−05 | 2.54E−03 |
| EEF1A2 | 1.41 | 1.24E−05 | 2.58E−03 |
| LGALS3BP | 1.17 | 1.30E−05 | 2.65E−03 |
| SPARC | 1.15 | 1.44E−05 | 2.72E−03 |
| CSPG4 | 1.29 | 1.68E−05 | 2.80E−03 |
| HLA-A | 1.30 | 1.61E−05 | 2.80E−03 |
| ISG20 | 1.20 | 1.71E−05 | 2.80E−03 |
| PDE3A | 2.14 | 1.58E−05 | 2.80E−03 |
| EPHA3 | 1.24 | 2.01E−05 | 3.04E−03 |
| GPR153 | 1.49 | 2.68E−05 | 3.40E−03 |
| PCSK1 | 1.81 | 2.51E−05 | 3.40E−03 |
| SFRP1 | 1.22 | 2.59E−05 | 3.40E−03 |
| TCN1 | 1.65 | 2.84E−05 | 3.46E−03 |
| PYGB | 1.20 | 2.99E−05 | 3.59E−03 |
| TRPV4 | 1.34 | 3.43E−05 | 3.90E−03 |
| VAT1L | 1.42 | 3.53E−05 | 3.96E−03 |
| ARNT2 | 1.41 | 3.62E−05 | 3.96E−03 |
| MIA | 1.27 | 3.60E−05 | 3.96E−03 |
| EEF1G | 1.07 | 3.74E−05 | 4.04E−03 |
| ATG101 | 1.08 | 4.04E−05 | 4.11E−03 |
| CUX1 | 1.12 | 3.94E−05 | 4.11E−03 |
| RPL10A | 1.06 | 4.03E−05 | 4.11E−03 |
| TESC | 1.63 | 3.90E−05 | 4.11E−03 |
| VGF | 1.73 | 3.98E−05 | 4.11E−03 |
| SLC24A3 | 1.73 | 4.32E−05 | 4.25E−03 |
| CEMIP | 1.41 | 4.50E−05 | 4.27E−03 |
| IGFBP3 | 1.58 | 4.54E−05 | 4.27E−03 |
| AMACR | 1.31 | 5.12E−05 | 4.41E−03 |
| CST3 | 1.18 | 5.75E−05 | 4.41E−03 |
| FAM210B | 1.28 | 5.54E−05 | 4.41E−03 |
| NR4A1 | 1.16 | 5.70E−05 | 4.41E−03 |
| PAPSS2 | 1.56 | 5.51E−05 | 4.41E−03 |
| PRAF2 | 1.07 | 5.61E−05 | 4.41E−03 |
| TGFA | 1.17 | 5.25E−05 | 4.41E−03 |
| GNAS | 1.28 | 5.90E−05 | 4.45E−03 |
| CSGALNACT1 | 1.43 | 6.22E−05 | 4.62E−03 |
| HLA-DRB1 | 1.27 | 6.21E−05 | 4.62E−03 |
| PLD3 | 1.22 | 6.44E−05 | 4.64E−03 |
| RPL10 | 1.03 | 6.35E−05 | 4.64E−03 |
| SERPINA3 | 1.78 | 6.47E−05 | 4.64E−03 |
| NES | 1.12 | 6.76E−05 | 4.71E−03 |
| PLOD3 | 1.18 | 6.84E−05 | 4.71E−03 |
| SNHG6 | 1.04 | 6.74E−05 | 4.71E−03 |
| TFPI2 | 1.34 | 6.94E−05 | 4.71E−03 |
| CHPF | 1.30 | 7.72E−05 | 5.11E−03 |
| EYA1 | 1.45 | 7.88E−05 | 5.11E−03 |
| SOX10 | 1.16 | 7.82E−05 | 5.11E−03 |
| CXCL8 | 1.77 | 8.34E−05 | 5.29E−03 |
| HTRA1 | 1.70 | 8.48E−05 | 5.35E−03 |
| LAMA4 | 1.41 | 9.17E−05 | 5.50E−03 |
| RIPK4 | 1.37 | 9.11E−05 | 5.50E−03 |
| TSPAN13 | 1.26 | 9.39E−05 | 5.55E−03 |
| ACSL3 | 1.16 | 9.77E−05 | 5.63E−03 |
| ABCA2 | 1.28 | 1.02E−04 | 5.67E−03 |
| PTPRU | 1.47 | 1.02E−04 | 5.67E−03 |
| UGGT2 | 1.08 | 1.02E−04 | 5.67E−03 |
| COL9A3 | 1.16 | 1.04E−04 | 5.67E−03 |
| ADGRL1 | 1.29 | 1.06E−04 | 5.70E−03 |
| ATOX1 | 1.14 | 1.06E−04 | 5.70E−03 |
| HLA-DQB1 | 1.33 | 1.06E−04 | 5.70E−03 |
| COL6A2 | 1.28 | 1.09E−04 | 5.74E−03 |
| PXYLP1 | 1.27 | 1.09E−04 | 5.74E−03 |
| KIAA0040 | 1.24 | 1.11E−04 | 5.76E−03 |
| SPRY4 | 1.30 | 1.12E−04 | 5.76E−03 |
| ABHD17C | 1.34 | 1.15E−04 | 5.87E−03 |
| FABP6 | 1.52 | 1.21E−04 | 6.02E−03 |
| NR4A2 | 1.29 | 1.23E−04 | 6.05E−03 |
| LRPAP1 | 1.15 | 1.30E−04 | 6.24E−03 |
| AREG | 1.83 | 1.34E−04 | 6.35E−03 |
| B3GNT7 | 1.43 | 1.35E−04 | 6.35E−03 |
| ACY1 | 1.21 | 1.36E−04 | 6.36E−03 |
| QSOX1 | 1.16 | 1.38E−04 | 6.41E−03 |
| SEZ6L2 | 1.26 | 1.46E−04 | 6.70E−03 |
| SLC25A23 | 1.27 | 1.47E−04 | 6.70E−03 |
| HLA-DRA | 1.19 | 1.51E−04 | 6.80E−03 |
| RPLPO | 1.06 | 1.52E−04 | 6.81E−03 |
| RPS3 | 1.10 | 1.55E−04 | 6.83E−03 |
| CTNNA1 | 1.08 | 1.62E−04 | 6.87E−03 |
| PRKCSH | 1.11 | 1.60E−04 | 6.87E−03 |
| RPL3 | 1.09 | 1.61E−04 | 6.87E−03 |
| BSG | 1.10 | 1.79E−04 | 6.91E−03 |
| EEF2 | 1.11 | 1.79E−04 | 6.91E−03 |
| FCRLA | 1.45 | 1.74E−04 | 6.91E−03 |
| FKBP10 | 1.19 | 1.70E−04 | 6.91E−03 |
| ID1 | 1.15 | 1.70E−04 | 6.91E−03 |
| PLPP4 | 1.42 | 1.76E−04 | 6.91E−03 |
| PSKH1 | 1.17 | 1.71E−04 | 6.91E−03 |
| RPS5 | 1.09 | 1.79E−04 | 6.91E−03 |
| SEPN1 | 1.14 | 1.64E−04 | 6.91E−03 |
| SLC1A4 | 1.67 | 1.70E−04 | 6.91E−03 |
| VAMP5 | 1.20 | 1.77E−04 | 6.91E−03 |
| APRT | 1.08 | 1.90E−04 | 6.99E−03 |
| SLC26A2 | 1.09 | 1.87E−04 | 6.99E−03 |
| TMEM2 | 1.21 | 1.83E−04 | 6.99E−03 |
| DBNDD1 | 1.38 | 1.98E−04 | 7.18E−03 |
| PSAP | 1.25 | 1.99E−04 | 7.22E−03 |
| HLA-DRB4 | 1.22 | 2.07E−04 | 7.24E−03 |
| LTBP3 | 1.21 | 2.04E−04 | 7.24E−03 |
| RPS14 | 1.11 | 2.15E−04 | 7.37E−03 |
| ST6GAL1 | 1.42 | 2.24E−04 | 7.53E−03 |
| GSK3B | 1.25 | 2.26E−04 | 7.56E−03 |
| SCARB1 | 1.11 | 2.26E−04 | 7.56E−03 |
| MARCKSL1 | 1.24 | 2.35E−04 | 7.76E−03 |
| ACTN1 | 1.11 | 2.38E−04 | 7.80E−03 |
| MELTF | 1.30 | 2.38E−04 | 7.80E−03 |
| FAM234A | 1.28 | 2.46E−04 | 7.81E−03 |
| PITPNCA | 1.35 | 2.43E−04 | 7.81E−03 |
| SYTL5 | 1.25 | 2.43E−04 | 7.81E−03 |
| TLR4 | 1.11 | 2.45E−04 | 7.81E−03 |
| WIPI1 | 1.30 | 2.53E−04 | 7.88E−03 |
| CYBA | 1.20 | 2.55E−04 | 7.91E−03 |
| RPLP1 | 1.08 | 2.59E−04 | 7.97E−03 |
| HMCN1 | 1.44 | 2.66E−04 | 8.08E−03 |
| P3H4 | 1.17 | 2.66E−04 | 8.08E−03 |
| SCARA5 | 1.50 | 2.70E−04 | 8.11E−03 |
| FDCPS | 2.79 | 2.95E−04 | 8.57E−03 |
| HLA-DPA1 | 1.16 | 2.97E−04 | 8.57E−03 |
| SPTBN2 | 1.13 | 2.94E−04 | 8.57E−03 |
| COL18A1 | 1.34 | 3.13E−04 | 8.86E−03 |
| ICAM | 1.37 | 3.13E−04 | 8.86E−03 |
| MEGF8 | 1.20 | 3.17E−04 | 8.91E−03 |
| SLC25A6 | 1.10 | 3.19E−04 | 8.91E−03 |
| ABHD2 | 1.13 | 3.24E−04 | 8.99E−03 |
| PNPLA2 | 1.18 | 3.31E−04 | 9.12E−03 |
| LY6E | 1.10 | 3.35E−04 | 9.19E−03 |
| ANKH | 1.45 | 3.48E−04 | 9.40E−03 |
| IGSF8 | 1.32 | 3.48E−04 | 9.40E−03 |
| PRDX2 | 1.14 | 3.48E−04 | 9.40E−03 |
| CTHRC1 | 1.49 | 3.53E−04 | 9.44E−03 |
| PTGFRN | 1.14 | 3.54E−04 | 9.44E−03 |
| TFAP2B | 1.59 | 3.66E−04 | 9.68E−03 |
| GAA | 1.40 | 3.74E−04 | 9.79E−03 |
| LOC102724122 | 1.46 | 3.84E−04 | 9.97E−03 |

TABLE 6
ChIP-seq characterization of JUNB target genes. Only genes
with JUNB ChIP-seq peaks that were significantly differentially
expressed as measured by RNA-seq are listed.

| Isoform | Fold | Pval | Padj |
|---|---|---|---|
| GALNT9 | 12426667.67 | 1.78E−06 | 4.85E−05 |
| KPRP | 404795 | 2.06E−06 | 5.32E−05 |
| PRR9 | 249944 | 7.42E−06 | 1.08E−04 |
| PLD5 | 1363.40 | 5.18E−06 | 8.67E−05 |
| PDPN | 807.85 | 3.36E−08 | 1.05E−05 |
| COL22A1 | 510.96 | 2.62E−08 | 9.89E−06 |
| LOC101927120 | 441.36 | 9.24E−05 | 5.14E−04 |
| PRRT4 | 350.61 | 2.66E−06 | 5.99E−05 |
| LCE1D | 272.05 | 2.88E−06 | 6.19E−05 |
| SERPINB2 | 218.85 | 1.78E−07 | 1.83E−05 |
| HMCN2 | 217.73 | 3.88E−06 | 7.29E−05 |
| SCEL | 191.82 | 2.71E−07 | 2.17E−05 |
| CCL3 | 189.83 | 4.30E−07 | 2.46E−05 |
| CXCL14 | 153.02 | 3.13E−07 | 2.21E−05 |
| MMP10 | 146.03 | 3.22E−07 | 2.25E−05 |
| HAS2 | 132.26 | 1.47E−08 | 8.56E−06 |
| PI3 | 108.57 | 1.36E−07 | 1.69E−05 |
| LRFN5 | 97.76 | 1.58E−09 | 3.61E−06 |
| ACAN | 79.24 | 1.70E−08 | 8.62E−06 |
| CDH6 | 78.69 | 3.12E−05 | 2.52E−04 |
| OPRL1 | 50.72 | 1.13E−06 | 3.98E−05 |
| CD24 | 49.60 | 2.02E−09 | 3.69E−06 |
| BCL2A1 | 44.67 | 2.56E−07 | 2.14E−05 |
| FERMT1 | 39.66 | 2.46E−08 | 9.89E−06 |
| JUNB | 33.35 | 1.89E−08 | 8.62E−06 |
| CTSS | 32.74 | 3.65E−07 | 2.32E−05 |
| BRINP2 | 31.46 | 4.41E−07 | 2.46E−05 |
| BTBD11 | 31.21 | 1.29E−07 | 1.69E−05 |
| CALB1 | 30.50 | 1.38E−05 | 1.55E−04 |
| ESM1 | 26.97 | 6.42E−08 | 1.25E−05 |
| PTGS2 | 26.54 | 4.90E−06 | 8.38E−05 |
| TAC1 | 23.46 | 4.49E−06 | 7.98E−05 |
| VCAN | 21.32 | 5.96E−08 | 1.25E−05 |
| CD93 | 21.10 | 1.62E−06 | 4.75E−05 |
| C15orf48 | 19.95 | 8.77E−06 | 1.18E−04 |
| MAST4 | 19.76 | 3.67E−08 | 1.08E−05 |
| SLAMF9 | 19.53 | 6.00E−07 | 2.78E−05 |
| MMP1 | 18.96 | 3.04E−07 | 2.21E−05 |
| EPAS1 | 18.84 | 1.07E−06 | 3.88E−05 |
| RAET1L | 16.57 | 1.91E−04 | 8.38E−04 |
| FLRT2 | 15.64 | 9.30E−08 | 1.41E−05 |
| DNER | 14.07 | 5.72E−05 | 3.70E−04 |
| LYPD3 | 13.96 | 6.18E−06 | 9.53E−05 |
| GALNT12 | 13.81 | 2.85E−06 | 6.19E−05 |
| BIRC3 | 13.78 | 8.77E−07 | 3.39E−05 |
| ALK | 13.46 | 6.04E−08 | 1.25E−05 |
| CXCL3 | 12.81 | 1.74E−05 | 1.73E−04 |
| MMP9 | 12.62 | 1.06E−04 | 5.65E−04 |
| GK | 12.31 | 6.51E−06 | 9.85E−05 |
| SERPINE1 | 12.25 | 8.68E−06 | 1.18E−04 |
| IKZF2 | 11.42 | 4.50E−06 | 7.98E−05 |
| DUSP1 | 11.09 | 6.96E−07 | 3.00E−05 |
| WSCD1 | 10.99 | 6.76E−07 | 2.95E−05 |
| PDZRN3 | 10.47 | 2.20E−07 | 2.06E−05 |
| KRT6B | 10.44 | 3.71E−09 | 4.23E−06 |
| NCAM1 | 10.30 | 4.02E−07 | 2.40E−05 |
| DSC2 | 10.29 | 2.64E−05 | 2.24E−04 |
| KIAA0040 | 10.25 | 5.99E−08 | 1.25E−05 |
| FAT3 | 9.68 | 1.46E−06 | 4.60E−05 |
| RPSAP52 | 9.34 | 1.27E−05 | 1.48E−04 |
| MDGA1 | 9.29 | 1.14E−04 | 5.87E−04 |
| HS3ST3B1 | 9.21 | 9.66E−05 | 5.30E−04 |
| WNT9A | 9.08 | 1.40E−06 | 4.43E−05 |
| TMCC2 | 8.48 | 2.33E−07 | 2.12E−05 |
| FAM19A2 | 8.41 | 1.09E−06 | 3.92E−05 |
| CXCL8 | 8.37 | 6.64E−07 | 2.93E−05 |
| LOC105372663 | 8.33 | 8.83E−05 | 4.98E−04 |
| KRTAP2-3 | 8.06 | 1.78E−06 | 4.85E−05 |
| USP43 | 7.95 | 7.66E−07 | 3.12E−05 |
| TGFBI | 7.94 | 3.47E−09 | 4.23E−06 |
| LAMB3 | 7.86 | 3.19E−08 | 1.05E−05 |
| ITGA2 | 7.77 | 2.71E−07 | 2.17E−05 |
| CLMP | 7.69 | 3.15E−07 | 2.21E−05 |
| MYH16 | 7.64 | 1.51E−05 | 1.61E−04 |
| INHBA | 7.57 | 6.79E−08 | 1.25E−05 |
| LEMD1 | 7.52 | 1.37E−07 | 1.69E−05 |
| ARNTL2 | 7.49 | 3.14E−08 | 1.05E−05 |
| PARM1 | 7.46 | 3.46E−06 | 6.77E−05 |
| IGFBP3 | 7.21 | 6.57E−08 | 1.25E−05 |
| XYLT1 | 7.16 | 3.73E−05 | 2.79E−04 |
| GAP43 | 7.05 | 6.04E−08 | 1.25E−05 |
| A4GALT | 6.74 | 6.97E−07 | 3.00E−05 |
| SYNC | 6.73 | 7.54E−06 | 1.09E−04 |
| SETBP1 | 6.61 | 3.68E−07 | 2.32E−05 |
| KCNC4 | 5.83 | 1.06E−04 | 5.64E−04 |
| CHMP4C | 5.62 | 2.01E−07 | 1.95E−05 |
| AHNAK2 | 5.55 | 8.96E−06 | 1.20E−04 |
| ARSB | 5.50 | 2.52E−06 | 5.85E−05 |
| COL6A3 | 5.41 | 1.53E−07 | 1.73E−05 |
| ZNF536 | 5.32 | 2.46E−06 | 5.82E−05 |
| KCNMA1 | 5.31 | 3.65E−05 | 2.74E−04 |
| NRCAM | 5.19 | 2.99E−07 | 2.21E−05 |
| LRIG1 | 5.05 | 1.69E−06 | 4.78E−05 |
| TLN2 | 5.05 | 1.63E−06 | 4.75E−05 |
| SLC14A1 | 4.99 | 8.60E−06 | 1.17E−04 |
| SH2D2A | 4.99 | 7.61E−05 | 4.48E−04 |
| RGS2 | 4.98 | 1.03E−05 | 1.30E−04 |
| SQRDL | 4.88 | 4.16E−05 | 2.99E−04 |
| GEM | 4.85 | 1.19E−06 | 4.10E−05 |
| HMOX1 | 4.79 | 1.49E−06 | 4.62E−05 |
| LIF | 4.70 | 7.21E−07 | 3.07E−05 |
| B3GAT1 | 4.67 | 1.12E−05 | 1.37E−04 |
| PKIA | 4.63 | 5.07E−07 | 2.56E−05 |
| KLF7 | 4.60 | 9.90E−07 | 3.66E−05 |
| C3orf67 | 4.58 | 3.39E−06 | 6.69E−05 |
| TIMP1 | 4.53 | 6.45E−07 | 2.87E−05 |
| SPSB4 | 4.49 | 3.15E−05 | 2.53E−04 |
| IL1RL1 | 4.46 | 2.50E−05 | 2.15E−04 |
| DSP | 4.45 | 5.79E−07 | 2.71E−05 |
| SPRR2D | 4.34 | 1.29E−05 | 1.49E−04 |
| MYO1D | 4.28 | 2.93E−07 | 2.21E−05 |
| IL24 | 4.26 | 1.59E−07 | 1.73E−05 |
| ARHGAP22 | 4.25 | 2.15E−04 | 9.04E−04 |
| NFASC | 4.11 | 9.89E−07 | 3.66E−05 |
| TMEM154 | 4.05 | 7.93E−06 | 1.13E−04 |
| RNF19B | 4.04 | 1.49E−06 | 4.62E−05 |
| NRIP3 | 4.02 | 1.53E−05 | 1.63E−04 |
| LINC00941 | 4.02 | 2.28E−06 | 5.53E−05 |
| GPR158 | 3.89 | 4.20E−05 | 3.00E−04 |
| BPGM | 3.84 | 1.53E−06 | 4.67E−05 |
| GRB10 | 3.83 | 1.66E−06 | 4.77E−05 |
| TMC7 | 3.72 | 2.58E−04 | 1.04E−03 |
| DIP2B | 3.72 | 1.60E−06 | 4.74E−05 |
| PLAT | 3.68 | 6.65E−06 | 9.96E−05 |
| CDYL2 | 3.68 | 1.24E−07 | 1.67E−05 |
| RAPH1 | 3.64 | 5.15E−06 | 8.65E−05 |
| IGSF9B | 3.63 | 2.09E−05 | 1.93E−04 |
| PPARD | 3.61 | 1.09E−05 | 1.35E−04 |
| PLEKHG5 | 3.60 | 2.66E−07 | 2.17E−05 |
| LPXN | 3.57 | 5.61E−07 | 2.68E−05 |
| ARIDSA | 3.52 | 3.14E−06 | 6.45E−05 |
| CCDC80 | 3.51 | 2.69E−06 | 6.02E−05 |
| LYPD1 | 3.47 | 4.97E−07 | 2.53E−05 |
| CD55 | 3.47 | 1.66E−07 | 1.75E−05 |
| MBOAT2 | 3.47 | 8.95E−07 | 3.43E−05 |
| LOC101928143 | 3.44 | 2.03E−04 | 8.74E−04 |
| RGMB | 3.41 | 1.79E−06 | 4.85E−05 |
| UCN2 | 3.40 | 3.77E−06 | 7.17E−05 |
| TMEM117 | 3.40 | 2.27E−05 | 2.04E−04 |
| ADGRG6 | 3.37 | 1.89E−05 | 1.81E−04 |
| HIST2H2BE | 3.36 | 1.74E−05 | 1.73E−04 |
| ALPK1 | 3.34 | 9.51E−06 | 1.25E−04 |
| LINC00704 | 3.33 | 3.15E−04 | 1.19E−03 |
| PITPNC1 | 3.32 | 1.28E−06 | 4.28E−05 |
| KCNG1 | 3.31 | 8.83E−08 | 1.37E−05 |
| ANGPTL2 | 3.27 | 1.11E−06 | 3.95E−05 |
| NREP | 3.27 | 4.18E−08 | 1.16E−05 |
| SDC1 | 3.25 | 3.67E−07 | 2.32E−05 |
| C1orf198 | 3.25 | 2.07E−06 | 5.32E−05 |
| TBX2 | 3.24 | 4.39E−07 | 2.46E−05 |
| IRF9 | 3.21 | 3.83E−04 | 1.39E−03 |
| CXCL1 | 3.20 | 3.01E−06 | 6.32E−05 |
| ELL2 | 3.19 | 1.78E−05 | 1.75E−04 |
| SPANXB1 | 3.18 | 1.29E−06 | 4.29E−05 |
| TMCC3 | 3.17 | 2.24E−06 | 5.50E−05 |
| S100A2 | 3.14 | 5.24E−07 | 2.60E−05 |
| TM4SF1 | 3.13 | 1.56E−06 | 4.67E−05 |
| ETHE1 | 3.12 | 9.66E−07 | 3.63E−05 |
| RGS20 | 3.10 | 1.81E−04 | 8.06E−04 |
| MYD88 | 3.09 | 1.74E−06 | 4.83E−05 |
| ANTXR2 | 3.06 | 1.92E−05 | 1.83E−04 |
| LOC105369340 | 3.06 | 2.61E−04 | 1.05E−03 |
| NRP1 | 3.06 | 1.90E−07 | 1.88E−05 |
| FBXO27 | 3.02 | 2.52E−07 | 2.13E−05 |
| IL4R | 3.01 | 7.46E−06 | 1.08E−04 |
| AJAP1 | 2.99 | 2.43E−05 | 2.11E−04 |
| PDGFC | 2.97 | 7.95E−05 | 4.62E−04 |
| HBEGF | 2.97 | 4.28E−05 | 3.04E−04 |
| BIRC2 | 2.95 | 7.23E−07 | 3.07E−05 |
| ID2 | 2.92 | 1.63E−05 | 1.68E−04 |
| ADAMTS1 | 2.91 | 5.56E−06 | 8.93E−05 |
| SATB1 | 2.91 | 6.37E−06 | 9.68E−05 |
| FOXC1 | 2.90 | 7.79E−06 | 1.11E−04 |
| LOC105376382 | 2.89 | 2.90E−04 | 1.13E−03 |
| SLC2A13 | 2.87 | 1.23E−04 | 6.22E−04 |
| GJB3 | 2.86 | 2.12E−05 | 1.95E−04 |
| NT5E | 2.84 | 4.65E−07 | 2.47E−05 |
| LOC100507002 | 2.84 | 3.21E−03 | 7.10E−03 |
| SPOCK1 | 2.83 | 1.36E−06 | 4.42E−05 |
| WNT5A | 2.76 | 3.75E−07 | 2.35E−05 |
| HELZ2 | 2.75 | 4.63E−03 | 9.42E−03 |
| PTPRM | 2.72 | 1.42E−04 | 6.82E−04 |
| VLDLR | 2.72 | 2.37E−05 | 2.09E−04 |
| HDAC9 | 2.71 | 1.33E−05 | 1.51E−04 |
| STRA6 | 2.71 | 7.38E−08 | 1.25E−05 |
| TMEM255B | 2.71 | 2.63E−03 | 6.04E−03 |
| CIT | 2.68 | 1.38E−06 | 4.42E−05 |
| HIST2H2AA3 | 2.66 | 4.46E−06 | 7.96E−05 |
| RNPEPL1 | 2.66 | 2.68E−05 | 2.26E−04 |
| GNG2 | 2.64 | 8.30E−06 | 1.15E−04 |
| CRYL1 | 2.64 | 1.97E−04 | 8.55E−04 |
| AKR1C2 | 2.62 | 8.56E−05 | 4.87E−04 |
| PHLDB3 | 2.61 | 3.01E−07 | 2.21E−05 |
| EFNB1 | 2.57 | 2.10E−05 | 1.94E−04 |
| FHL2 | 2.57 | 4.63E−07 | 2.47E−05 |
| APOD | 2.56 | 1.66E−04 | 7.63E−04 |
| SAT1 | 2.54 | 4.76E−05 | 3.26E−04 |
| ARHGEF4 | 2.54 | 1.24E−04 | 6.26E−04 |
| ADGRE2 | 2.53 | 4.94E−06 | 8.43E−05 |
| RASA1 | 2.52 | 1.83E−05 | 1.78E−04 |
| S100A16 | 2.52 | 3.80E−08 | 1.08E−05 |
| RNF182 | 2.51 | 5.39E−05 | 3.55E−04 |
| EDNRB | 2.51 | 6.54E−05 | 4.06E−04 |
| STEAP3 | 2.51 | 1.34E−07 | 1.69E−05 |
| SEMA3A | 2.50 | 9.00E−06 | 1.20E−04 |
| HIST1H2BK | 2.50 | 1.43E−04 | 6.84E−04 |
| ETS1 | 2.47 | 4.67E−06 | 8.19E−05 |
| PLAUR | 2.46 | 1.67E−06 | 4.78E−05 |
| ENDOD1 | 2.46 | 4.66E−05 | 3.23E−04 |
| NKILA | 2.45 | 2.76E−04 | 1.08E−03 |
| RELB | 2.45 | 1.16E−05 | 1.40E−04 |
| FAM107B | 2.44 | 8.70E−05 | 4.93E−04 |
| CD59 | 2.40 | 5.19E−08 | 1.25E−05 |
| F2RL2 | 2.40 | 1.65E−05 | 1.68E−04 |
| PCOLCE2 | 2.39 | 4.95E−04 | 1.69E−03 |
| PFKFB4 | 2.39 | 1.68E−06 | 4.78E−05 |
| MANBA | 2.39 | 5.70E−06 | 9.11E−05 |
| HIVEP2 | 2.38 | 7.42E−07 | 3.09E−05 |
| CDK17 | 2.38 | 4.23E−06 | 7.73E−05 |
| NFKB2 | 2.38 | 6.28E−07 | 2.84E−05 |
| PLS1 | 2.37 | 4.08E−05 | 2.95E−04 |
| FLJ20021 | 2.37 | 4.09E−04 | 1.46E−03 |
| GDNF | 2.36 | 1.58E−04 | 7.38E−04 |
| ID1 | 2.36 | 2.96E−06 | 6.25E−05 |
| TNFAIP3 | 2.34 | 2.30E−05 | 2.05E−04 |
| DLX2 | 2.34 | 6.13E−05 | 3.88E−04 |
| TDRD7 | 2.34 | 3.53E−05 | 2.70E−04 |
| PLXNA3 | 2.34 | 1.11E−05 | 1.36E−04 |
| SMAD7 | 2.34 | 1.20E−03 | 3.27E−03 |
| PTHLH | 2.32 | 1.45E−04 | 6.91E−04 |
| ARG2 | 2.32 | 1.75E−04 | 7.89E−04 |
| FOXC2 | 2.31 | 4.16E−04 | 1.48E−03 |
| AQP3 | 2.31 | 3.38E−04 | 1.26E−03 |
| SOX4 | 2.30 | 1.80E−05 | 1.76E−04 |
| TMBIM1 | 2.29 | 1.28E−06 | 4.28E−05 |
| ADAM19 | 2.29 | 4.84E−07 | 2.51E−05 |
| PRKCDBP | 2.29 | 8.05E−05 | 4.66E−04 |
| NFKBIZ | 2.28 | 7.46E−06 | 1.08E−04 |
| ADK | 2.27 | 5.09E−06 | 8.59E−05 |
| SREK1IP1 | 2.26 | 1.61E−06 | 4.75E−05 |
| ITGAS | 2.26 | 1.39E−06 | 4.43E−05 |
| FAS | 2.25 | 4.96E−06 | 8.44E−05 |
| HSPG2 | 2.25 | 6.23E−06 | 9.56E−05 |
| IFNGR1 | 2.24 | 1.31E−06 | 4.29E−05 |
| KDM6B | 2.24 | 2.44E−07 | 2.12E−05 |
| FRMD3 | 2.23 | 5.84E−05 | 3.76E−04 |
| CREM | 2.23 | 1.82E−04 | 8.11E−04 |
| PMEPA1 | 2.23 | 2.61E−07 | 2.15E−05 |
| CD68 | 2.23 | 1.57E−05 | 1.65E−04 |
| SERINCS | 2.22 | 7.19E−05 | 4.31E−04 |
| TRNP1 | 2.22 | 1.85E−06 | 4.95E−05 |
| SYNJ2 | 2.21 | 4.50E−06 | 7.98E−05 |
| RAP2B | 2.20 | 6.89E−05 | 4.21E−04 |
| HOMER2 | 2.19 | 3.90E−04 | 1.40E−03 |
| S100A1 | 2.19 | 3.64E−07 | 2.32E−05 |
| AMOTL1 | 2.18 | 2.42E−07 | 2.12E−05 |
| APC | 2.18 | 8.04E−06 | 1.13E−04 |
| LOC100996740 | 2.18 | 4.69E−06 | 8.19E−05 |
| MGST3 | 2.17 | 5.53E−05 | 3.61E−04 |
| TLDC1 | 2.17 | 4.91E−05 | 3.32E−04 |
| PORCN | 2.16 | 8.57E−05 | 4.88E−04 |
| SMPD1 | 2.16 | 3.11E−05 | 2.51E−04 |
| RIN2 | 2.15 | 2.68E−04 | 1.07E−03 |
| MAP1B | 2.15 | 2.20E−06 | 5.45E−05 |
| FRMD4B | 2.15 | 2.20E−05 | 2.00E−04 |
| ERGIC1 | 2.14 | 3.91E−07 | 2.36E−05 |
| STIM1 | 2.13 | 1.62E−06 | 4.75E−05 |
| SORBS2 | 2.13 | 3.31E−06 | 6.64E−05 |
| SDC2 | 2.13 | 1.03E−05 | 1.30E−04 |
| CTHRC1 | 2.13 | 1.17E−04 | 5.98E−04 |
| CEND1 | 2.12 | 3.11E−05 | 2.51E−04 |
| SMTN | 2.12 | 1.97E−06 | 5.14E−05 |
| KLF13 | 2.10 | 2.34E−05 | 2.07E−04 |
| ABLIM1 | 2.09 | 1.15E−04 | 5.91E−04 |
| F11R | 2.08 | 2.58E−05 | 2.21E−04 |
| LINC00707 | 2.08 | 3.61E−05 | 2.72E−04 |
| DBNDD1 | 2.05 | 5.30E−06 | 8.71E−05 |
| ITGB8 | 2.04 | 1.01E−05 | 1.30E−04 |
| COL9A2 | 2.04 | 1.72E−05 | 1.72E−04 |
| DHRS7 | 2.03 | 3.56E−04 | 1.31E−03 |
| RAB11FIP5 | 2.03 | 6.52E−06 | 9.85E−05 |
| ACSL1 | 2.03 | 2.24E−05 | 2.02E−04 |
| NR4A1 | 2.02 | 3.78E−07 | 2.35E−05 |
| AHCYL2 | 2.02 | 2.45E−05 | 2.13E−04 |
| TMEM132A | 2.02 | 9.98E−06 | 1.29E−04 |
| SHC4 | 2.02 | 3.08E−05 | 2.50E−04 |
| PANX1 | 2.02 | 1.02E−05 | 1.30E−04 |
| HIST1H2AC | 2.02 | 9.47E−04 | 2.72E−03 |
| MLLT3 | 2.01 | 2.62E−04 | 1.05E−03 |
| CD82 | 2.01 | 9.13E−05 | 5.10E−04 |
| NFIB | 2.01 | 8.44E−06 | 1.16E−04 |
| STK17A | 2.00 | 2.43E−05 | 2.11E−04 |
| NRP2 | 1.99 | 1.36E−06 | 4.42E−05 |
| ABHD2 | 1.99 | 4.44E−07 | 2.46E−05 |
| SIPA1L1 | 1.98 | 3.65E−04 | 1.34E−03 |
| PGM3 | 1.98 | 2.42E−04 | 9.90E−04 |
| CAPRIN2 | 1.98 | 2.64E−03 | 6.06E−03 |
| NTM | 1.97 | 8.95E−04 | 2.60E−03 |
| ARSA | 1.97 | 2.53E−03 | 5.85E−03 |
| FAM214B | 1.96 | 1.59E−05 | 1.67E−04 |
| UBA6 | 1.96 | 2.88E−04 | 1.12E−03 |
| HIST3H2A | 1.96 | 2.69E−04 | 1.07E−03 |
| IL13RA1 | 1.96 | 7.37E−06 | 1.08E−04 |
| TBC1D9 | 1.96 | 1.49E−04 | 7.08E−04 |
| GPRC5A | 1.96 | 1.70E−06 | 4.78E−05 |
| KIF13A | 1.95 | 6.39E−05 | 3.99E−04 |
| MXRA7 | 1.95 | 5.52E−06 | 8.88E−05 |
| NET1 | 1.95 | 1.30E−05 | 1.49E−04 |
| CTSL | 1.95 | 9.73E−06 | 1.27E−04 |
| JARID2 | 1.95 | 2.53E−05 | 2.17E−04 |
| RIPK2 | 1.95 | 1.19E−05 | 1.42E−04 |
| PIK3CD | 1.94 | 9.67E−06 | 1.27E−04 |
| CCDC71L | 1.93 | 1.76E−06 | 4.85E−05 |
| PRNP | 1.93 | 4.92E−06 | 8.40E−05 |
| NPDC1 | 1.93 | 1.15E−04 | 5.89E−04 |
| DIEXF | 1.93 | 1.52E−05 | 1.62E−04 |
| PLPP4 | 1.92 | 1.40E−04 | 6.79E−04 |
| TRPV4 | 1.92 | 4.81E−06 | 8.31E−05 |
| ABTB1 | 1.92 | 3.87E−04 | 1.40E−03 |
| LTBP4 | 1.91 | 1.28E−05 | 1.48E−04 |
| SOCS2 | 1.91 | 2.37E−03 | 5.56E−03 |
| TOMM34 | 1.91 | 1.49E−06 | 4.62E−05 |
| RABAC1 | 1.91 | 1.44E−05 | 1.57E−04 |
| MET | 1.91 | 5.12E−07 | 2.56E−05 |
| LINC00623 | 1.90 | 4.88E−05 | 3.31E−04 |
| ATOX1 | 1.90 | 4.62E−07 | 2.47E−05 |
| RHOC | 1.90 | 1.19E−07 | 1.62E−05 |
| GAS6 | 1.90 | 2.39E−06 | 5.70E−05 |
| MYO10 | 1.89 | 1.19E−04 | 6.06E−04 |
| TWF1 | 1.89 | 3.39E−06 | 6.69E−05 |
| RIPK4 | 1.89 | 4.23E−05 | 3.01E−04 |
| SH3PXD2B | 1.89 | 6.58E−06 | 9.89E−05 |
| NUP50 | 1.89 | 2.07E−06 | 5.32E−05 |
| CORO1C | 1.89 | 5.48E−06 | 8.84E−05 |
| GPR161 | 1.89 | 1.46E−04 | 6.95E−04 |
| CDK5R1 | 1.88 | 2.63E−06 | 5.95E−05 |
| PERP | 1.88 | 1.40E−06 | 4.43E−05 |
| MYH9 | 1.88 | 2.98E−07 | 2.21E−05 |
| GZF1 | 1.88 | 1.56E−03 | 4.01E−03 |
| MROH1 | 1.88 | 6.77E−05 | 4.17E−04 |
| APBB2 | 1.87 | 3.15E−05 | 2.53E−04 |
| SNX9 | 1.87 | 1.54E−05 | 1.63E−04 |
| UBA6-AS1 | 1.87 | 1.05E−04 | 5.60E−04 |
| FURIN | 1.87 | 6.48E−08 | 1.25E−05 |
| KLF6 | 1.87 | 1.41E−04 | 6.81E−04 |
| RUNX1 | 1.87 | 2.40E−05 | 2.10E−04 |
| DHRS1 | 1.86 | 1.21E−04 | 6.12E−04 |
| ECM1 | 1.85 | 1.81E−05 | 1.77E−04 |
| S100A11 | 1.85 | 2.36E−06 | 5.67E−05 |
| PARP8 | 1.85 | 1.64E−05 | 1.68E−04 |
| GADD45B | 1.85 | 1.02E−04 | 5.54E−04 |
| DLGAP4 | 1.84 | 5.76E−06 | 9.19E−05 |
| TAF9B | 1.84 | 1.53E−05 | 1.63E−04 |
| VAT1L | 1.84 | 1.27E−04 | 6.32E−04 |
| RHOB | 1.84 | 3.23E−06 | 6.57E−05 |
| SASH1 | 1.84 | 5.67E−06 | 9.09E−05 |
| PPP1R21 | 1.82 | 3.21E−04 | 1.21E−03 |
| MELTF | 1.82 | 3.08E−06 | 6.38E−05 |
| TAGLN2 | 1.82 | 5.22E−08 | 1.25E−05 |
| RASA2 | 1.82 | 4.28E−04 | 1.51E−03 |
| MAP1S | 1.80 | 2.49E−06 | 5.83E−05 |
| PRKCA | 1.80 | 1.47E−05 | 1.59E−04 |
| C9orf72 | 1.80 | 1.21E−03 | 3.28E−03 |
| PPIF | 1.80 | 8.05E−09 | 7.11E−06 |
| TJP1 | 1.80 | 5.83E−06 | 9.25E−05 |
| TRIO | 1.79 | 4.69E−06 | 8.19E−05 |
| APAF1 | 1.79 | 1.49E−05 | 1.60E−04 |
| SHROOM4 | 1.78 | 7.04E−05 | 4.26E−04 |
| ACAA1 | 1.78 | 1.64E−05 | 1.68E−04 |
| ZFYVE16 | 1.77 | 7.76E−05 | 4.55E−04 |
| TRIM58 | 1.77 | 3.77E−05 | 2.81E−04 |
| PINK1 | 1.77 | 4.99E−04 | 1.70E−03 |
| TBC1D19 | 1.76 | 2.05E−03 | 4.95E−03 |
| TRPS1 | 1.76 | 9.89E−05 | 5.38E−04 |
| RNF144A | 1.76 | 4.82E−06 | 8.31E−05 |
| CNKSR3 | 1.76 | 1.58E−04 | 7.37E−04 |
| TM4SF19 | 1.76 | 9.60E−05 | 5.29E−04 |
| SLC39A6 | 1.76 | 3.20E−06 | 6.54E−05 |
| HES1 | 1.75 | 1.11E−04 | 5.80E−04 |
| CPEB4 | 1.75 | 1.07E−05 | 1.33E−04 |
| ABRACL | 1.75 | 7.53E−06 | 1.09E−04 |
| AKAP12 | 1.74 | 7.74E−07 | 3.13E−05 |
| BCAS3 | 1.74 | 1.18E−03 | 3.23E−03 |
| DNAJB9 | 1.74 | 1.40E−04 | 6.79E−04 |
| NDRG1 | 1.74 | 4.54E−06 | 8.01E−05 |
| ZSWIM4 | 1.74 | 1.60E−05 | 1.67E−04 |
| SNX25 | 1.74 | 3.62E−05 | 2.73E−04 |
| STARD13 | 1.74 | 1.00E−04 | 5.45E−04 |
| PTPRU | 1.74 | 4.12E−05 | 2.97E−04 |
| IRGQ | 1.73 | 2.26E−05 | 2.03E−04 |
| NFKB1 | 1.72 | 7.82E−06 | 1.11E−04 |
| AGAP2-AS1 | 1.71 | 1.81E−05 | 1.77E−04 |
| ACOT7 | 1.71 | 1.59E−07 | 1.73E−05 |
| TXNIP | 1.71 | 2.77E−04 | 1.09E−03 |
| NOV | 1.71 | 9.51E−04 | 2.73E−03 |
| ARRDC1 | 1.71 | 2.22E−05 | 2.01E−04 |
| TMEM8A | 1.71 | 9.13E−05 | 5.10E−04 |
| PRDM2 | 1.71 | 3.20E−05 | 2.55E−04 |
| SDSL | 1.70 | 4.13E−04 | 1.47E−03 |
| UAP1 | 1.70 | 1.61E−05 | 1.67E−04 |
| KCNN4 | 1.70 | 7.27E−05 | 4.34E−04 |
| JOSD1 | 1.69 | 2.37E−06 | 5.68E−05 |
| DVL1 | 1.69 | 2.12E−05 | 1.94E−04 |
| ANKRA2 | 1.69 | 1.34E−04 | 6.56E−04 |
| MYADM | 1.69 | 1.20E−06 | 4.11E−05 |
| TGFBR1 | 1.69 | 2.41E−05 | 2.10E−04 |
| DUSP10 | 1.69 | 2.31E−05 | 2.05E−04 |
| UNC13B | 1.68 | 4.29E−04 | 1.51E−03 |
| FMN2 | 1.68 | 9.43E−04 | 2.71E−03 |
| RRAS | 1.68 | 9.07E−05 | 5.08E−04 |
| C14orf159 | 1.68 | 1.04E−03 | 2.93E−03 |
| MAFK | 1.68 | 1.31E−04 | 6.47E−04 |
| FZD8 | 1.68 | 2.24E−05 | 2.02E−04 |
| GDI1 | 1.68 | 1.81E−05 | 1.77E−04 |
| SLC18B1 | 1.68 | 5.64E−04 | 1.85E−03 |
| FYN | 1.67 | 4.48E−07 | 2.46E−05 |
| LOC102724122 | 1.67 | 1.10E−04 | 5.78E−04 |
| CHST7 | 1.67 | 2.11E−04 | 8.97E−04 |
| CENPM | 1.67 | 1.15E−04 | 5.89E−04 |
| HOMER3 | 1.67 | 2.88E−05 | 2.39E−04 |
| SLC22A4 | 1.67 | 2.02E−03 | 4.90E−03 |
| ADD2 | 1.67 | 1.70E−04 | 7.75E−04 |
| IL1RAPL1 | 1.66 | 8.27E−04 | 2.44E−03 |
| AIG1 | 1.66 | 1.79E−03 | 4.45E−03 |
| MSN | 1.66 | 2.71E−08 | 9.89E−06 |
| SLC9A3R2 | 1.66 | 3.01E−05 | 2.45E−04 |
| CORO2B | 1.66 | 9.79E−06 | 1.27E−04 |
| ARSD | 1.65 | 3.37E−04 | 1.26E−03 |
| SLC1A1 | 1.65 | 7.56E−04 | 2.29E−03 |
| HYAL3 | 1.65 | 1.35E−03 | 3.57E−03 |
| TRIB1 | 1.65 | 5.09E−06 | 8.59E−05 |
| SLC24A3 | 1.64 | 2.94E−05 | 2.42E−04 |
| ENTPD7 | 1.64 | 1.25E−04 | 6.27E−04 |
| SERPINB8 | 1.64 | 1.15E−05 | 1.40E−04 |
| FOXN2 | 1.64 | 8.63E−05 | 4.90E−04 |
| ABR | 1.64 | 4.46E−07 | 2.46E−05 |
| GLRX | 1.64 | 6.59E−05 | 4.07E−04 |
| PLSCR3 | 1.64 | 1.30E−05 | 1.49E−04 |
| OXSR1 | 1.63 | 3.71E−06 | 7.10E−05 |
| ACTA2 | 1.63 | 4.77E−04 | 1.64E−03 |
| RAP1GAP2 | 1.63 | 2.73E−04 | 1.07E−03 |
| NAGK | 1.63 | 5.00E−05 | 3.36E−04 |
| ARHGAP21 | 1.63 | 1.03E−05 | 1.30E−04 |
| TGFB1 | 1.62 | 2.92E−05 | 2.41E−04 |
| MFHAS1 | 1.62 | 6.83E−05 | 4.18E−04 |
| RAI14 | 1.62 | 6.89E−05 | 4.21E−04 |
| PITPNM2 | 1.62 | 1.02E−03 | 2.87E−03 |
| SPRY2 | 1.62 | 6.35E−06 | 9.68E−05 |
| CHPF | 1.61 | 2.25E−05 | 2.02E−04 |
| MSANTD3 | 1.61 | 2.72E−04 | 1.07E−03 |
| SEZ6L2 | 1.61 | 8.02E−06 | 1.13E−04 |
| GRN | 1.61 | 2.19E−06 | 5.45E−05 |
| ARPC1B | 1.61 | 2.41E−06 | 5.74E−05 |
| RNF215 | 1.61 | 9.82E−05 | 5.37E−04 |
| TICAM1 | 1.61 | 4.63E−04 | 1.61E−03 |
| PSAP | 1.61 | 2.48E−06 | 5.82E−05 |
| NAT6 | 1.61 | 2.45E−03 | 5.71E−03 |
| EFL1 | 1.60 | 6.18E−05 | 3.90E−04 |
| CEP170B | 1.60 | 1.10E−04 | 5.78E−04 |
| BTBD10 | 1.60 | 5.11E−06 | 8.61E−05 |
| CCNDBP1 | 1.60 | 1.11E−04 | 5.81E−04 |
| ASAH1 | 1.60 | 4.37E−05 | 3.08E−04 |
| IFI27L2 | 1.60 | 1.54E−03 | 3.97E−03 |
| TRIM35 | 1.60 | 1.22E−03 | 3.29E−03 |
| TMSB10 | 1.60 | 1.73E−06 | 4.82E−05 |
| P4HA2 | 1.59 | 3.74E−04 | 1.36E−03 |
| UBALD2 | 1.59 | 2.52E−04 | 1.02E−03 |
| CYSTM1 | 1.59 | 2.12E−03 | 5.10E−03 |
| CLSTN1 | 1.59 | 1.55E−05 | 1.64E−04 |
| TRAM2 | 1.59 | 6.71E−06 | 1.00E−04 |
| SLC39A8 | 1.59 | 2.12E−03 | 5.10E−03 |
| CAPN1 | 1.59 | 3.13E−06 | 6.44E−05 |
| MTMR1 | 1.59 | 1.81E−04 | 8.06E−04 |
| CLCF1 | 1.59 | 2.70E−04 | 1.07E−03 |
| SMYD2 | 1.59 | 3.11E−04 | 1.18E−03 |
| FOXD1 | 1.58 | 1.66E−05 | 1.68E−04 |
| GALNT7 | 1.58 | 8.79E−05 | 4.97E−04 |
| TMEM54 | 1.58 | 1.62E−04 | 7.51E−04 |
| MYL6 | 1.58 | 4.94E−07 | 2.53E−05 |
| INPP5A | 1.58 | 2.65E−04 | 1.06E−03 |
| DNASE1L1 | 1.58 | 1.60E−03 | 4.10E−03 |
| SLC39A13 | 1.58 | 1.97E−04 | 8.56E−04 |
| PHKA1 | 1.58 | 1.44E−04 | 6.90E−04 |
| 9-Mar | 1.58 | 2.01E−04 | 8.65E−04 |
| KCTD11 | 1.58 | 1.62E−03 | 4.12E−03 |
| EDEM1 | 1.58 | 8.33E−06 | 1.15E−04 |
| BCAS4 | 1.58 | 1.42E−04 | 6.84E−04 |
| IFT80 | 1.58 | 1.54E−04 | 7.24E−04 |
| MFGE8 | 1.57 | 4.10E−04 | 1.46E−03 |
| SPHK1 | 1.57 | 4.96E−04 | 1.69E−03 |
| LRP10 | 1.57 | 5.24E−06 | 8.68E−05 |
| PSEN2 | 1.57 | 1.45E−04 | 6.94E−04 |
| TMEM102 | 1.57 | 7.15E−04 | 2.20E−03 |
| APLP2 | 1.56 | 3.38E−05 | 2.64E−04 |
| ZCCHC14 | 1.56 | 1.52E−04 | 7.17E−04 |
| IL11 | 1.56 | 1.51E−04 | 7.14E−04 |
| ABAT | 1.56 | 1.01E−03 | 2.87E−03 |
| AIDA | 1.56 | 1.72E−04 | 7.79E−04 |
| EFNB2 | 1.56 | 1.05E−05 | 1.31E−04 |
| AP1S2 | 1.56 | 2.98E−05 | 2.43E−04 |
| CAPS | 1.56 | 2.01E−05 | 1.88E−04 |
| RELT | 1.55 | 9.70E−04 | 2.77E−03 |
| CLDN4 | 1.55 | 4.72E−04 | 1.63E−03 |
| ZFP36 | 1.55 | 1.12E−03 | 3.10E−03 |
| CLASP1 | 1.55 | 1.88E−05 | 1.80E−04 |
| MDFIC | 1.55 | 2.53E−03 | 5.87E−03 |
| BIVM | 1.55 | 1.41E−05 | 1.57E−04 |
| UBL3 | 1.55 | 1.07E−04 | 5.65E−04 |
| MAML2 | 1.55 | 2.29E−04 | 9.48E−04 |
| GJB1 | 1.55 | 7.95E−06 | 1.13E−04 |
| SLC33A1 | 1.55 | 6.80E−04 | 2.13E−03 |
| NPC1 | 1.54 | 2.90E−06 | 6.19E−05 |
| APPL2 | 1.54 | 3.64E−05 | 2.74E−04 |
| SERINC3 | 1.54 | 1.58E−05 | 1.66E−04 |
| GNAI1 | 1.54 | 1.30E−03 | 3.47E−03 |
| KIF21A | 1.54 | 3.21E−04 | 1.21E−03 |
| WLS | 1.54 | 2.77E−06 | 6.11E−05 |
| PARP3 | 1.54 | 8.18E−05 | 4.71E−04 |
| TEX30 | 1.54 | 1.63E−03 | 4.15E−03 |
| RALGPS2 | 1.54 | 7.05E−04 | 2.19E−03 |
| S100A13 | 1.54 | 7.96E−07 | 3.17E−05 |
| MAP1LC3B | 1.53 | 9.79E−06 | 1.27E−04 |
| RAB11FIP1 | 1.53 | 7.48E−05 | 4.44E−04 |
| PLD3 | 1.53 | 7.50E−05 | 4.44E−04 |
| YPEL5 | 1.53 | 4.20E−04 | 1.49E−03 |
| SLC35C1 | 1.53 | 8.12E−05 | 4.68E−04 |
| NR2F2 | 1.53 | 3.75E−05 | 2.80E−04 |
| USP11 | 1.53 | 2.84E−06 | 6.19E−05 |
| CLU | 1.53 | 2.38E−04 | 9.78E−04 |
| TCF7L2 | 1.53 | 9.53E−05 | 5.27E−04 |
| UBE2Q2 | 1.53 | 1.03E−05 | 1.30E−04 |
| ANKRD10 | 1.53 | 5.63E−04 | 1.85E−03 |
| FYCO1 | 1.53 | 1.72E−04 | 7.79E−04 |
| ZEB1 | 1.53 | 6.35E−06 | 9.68E−05 |
| SP3 | 1.53 | 2.95E−05 | 2.42E−04 |
| SRSF8 | 1.53 | 1.09E−05 | 1.35E−04 |
| HIST2H4A | 1.53 | 4.54E−03 | 9.28E−03 |
| HIST2H4B | 1.53 | 4.54E−03 | 9.28E−03 |
| YPEL3 | 1.53 | 3.16E−04 | 1.20E−03 |
| ANKS6 | 1.52 | 1.52E−04 | 7.18E−04 |
| SERGEF | 1.52 | 1.54E−04 | 7.26E−04 |
| RPRD1A | 1.52 | 9.77E−04 | 2.79E−03 |
| PTRH1 | 1.52 | 1.53E−03 | 3.95E−03 |
| UPP1 | 1.52 | 3.47E−04 | 1.29E−03 |
| TRPT1 | 1.52 | 2.35E−04 | 9.67E−04 |
| TTPAL | 1.52 | 1.96E−07 | 1.92E−05 |
| HEBP2 | 1.52 | 2.01E−03 | 4.88E−03 |
| SVIL | 1.51 | 7.05E−05 | 4.26E−04 |
| FEZ1 | 1.51 | 1.97E−06 | 5.14E−05 |
| SLCO4A1-AS1 | 1.51 | 1.19E−03 | 3.24E−03 |
| FUT8 | 1.51 | 2.74E−04 | 1.08E−03 |
| OSTF1 | 1.50 | 6.04E−04 | 1.95E−03 |
| LAP3 | 1.50 | 2.59E−04 | 1.04E−03 |
| PDE4A | 1.50 | 5.05E−05 | 3.38E−04 |
| SLC35G2 | 1.50 | 3.95E−04 | 1.42E−03 |
| UPRT | 1.50 | 2.58E−04 | 1.04E−03 |
| ISCA1 | 1.50 | 1.30E−04 | 6.42E−04 |
| FAM177A1 | 1.50 | 3.74E−03 | 7.98E−03 |
| SWAP70 | 1.49 | 1.75E−05 | 1.74E−04 |
| DSTYK | 1.49 | 6.20E−06 | 9.53E−05 |
| SP100 | 1.49 | 1.37E−03 | 3.62E−03 |
| DENND1A | 1.49 | 1.25E−05 | 1.47E−04 |
| SKAP2 | 1.49 | 1.67E−04 | 7.65E−04 |
| SKIL | 1.49 | 5.55E−04 | 1.83E−03 |
| POLD4 | 1.49 | 1.27E−04 | 6.32E−04 |
| SPRYD3 | 1.49 | 2.32E−03 | 5.46E−03 |
| CACNB3 | 1.48 | 6.57E−05 | 4.07E−04 |
| FAM83G | 1.48 | 3.58E−05 | 2.72E−04 |
| KHNYN | 1.48 | 2.34E−04 | 9.62E−04 |
| ABCA2 | 1.48 | 2.76E−05 | 2.30E−04 |
| MED13 | 1.48 | 5.40E−05 | 3.55E−04 |
| BCORL1 | 1.48 | 3.61E−03 | 7.75E−03 |
| ITPKB | 1.48 | 1.71E−03 | 4.31E−03 |
| PARVB | 1.48 | 7.30E−06 | 1.07E−04 |
| FAM168A | 1.47 | 4.36E−05 | 3.07E−04 |
| KIFAP3 | 1.47 | 1.86E−04 | 8.24E−04 |
| HIPK3 | 1.47 | 1.96E−04 | 8.52E−04 |
| FLOT1 | 1.47 | 5.97E−05 | 3.81E−04 |
| MAP3K4 | 1.47 | 1.26E−04 | 6.30E−04 |
| SERTAD1 | 1.47 | 3.87E−03 | 8.20E−03 |
| MANEAL | 1.47 | 2.61E−04 | 1.05E−03 |
| TCIRG1 | 1.47 | 5.12E−04 | 1.73E−03 |
| PLGRKT | 1.47 | 3.60E−04 | 1.33E−03 |
| QSOX1 | 1.47 | 5.09E−05 | 3.40E−04 |
| FGFR1 | 1.46 | 1.15E−04 | 5.89E−04 |
| PSEN1 | 1.46 | 4.72E−04 | 1.63E−03 |
| NOMO2 | 1.46 | 3.75E−05 | 2.80E−04 |
| SPAG9 | 1.46 | 1.01E−04 | 5.48E−04 |
| TMEM14A | 1.46 | 4.01E−03 | 8.43E−03 |
| NUMB | 1.46 | 1.84E−05 | 1.78E−04 |
| ATP2B4 | 1.46 | 1.83E−05 | 1.78E−04 |
| EVIS | 1.46 | 2.56E−03 | 5.90E−03 |
| DECR1 | 1.46 | 1.74E−03 | 4.36E−03 |
| ADIPOR1 | 1.45 | 1.55E−06 | 4.67E−05 |
| MICAL1 | 1.45 | 7.27E−04 | 2.23E−03 |
| PGPEP1 | 1.45 | 9.86E−04 | 2.81E−03 |
| STXBP1 | 1.45 | 3.24E−05 | 2.56E−04 |
| DOCK5 | 1.45 | 4.48E−04 | 1.57E−03 |
| STEAP1B | 1.44 | 1.01E−04 | 5.48E−04 |
| SLC6A15 | 1.44 | 2.65E−05 | 2.24E−04 |
| GM2A | 1.44 | 1.28E−04 | 6.37E−04 |
| CITED4 | 1.44 | 1.89E−03 | 4.65E−03 |
| PPTC7 | 1.44 | 8.22E−05 | 4.72E−04 |
| UHRF2 | 1.44 | 1.33E−03 | 3.54E−03 |
| LRFN4 | 1.44 | 5.78E−04 | 1.89E−03 |
| TMEM134 | 1.44 | 1.25E−03 | 3.37E−03 |
| SLC1A4 | 1.44 | 1.36E−04 | 6.66E−04 |
| IFI30 | 1.43 | 7.03E−05 | 4.26E−04 |
| CALCOCO1 | 1.43 | 1.07E−04 | 5.65E−04 |
| IER5 | 1.43 | 1.99E−04 | 8.60E−04 |
| MAPKBP1 | 1.43 | 4.43E−05 | 3.10E−04 |
| MAFF | 1.43 | 2.09E−03 | 5.02E−03 |
| CUX1 | 1.43 | 2.32E−05 | 2.06E−04 |
| PPP4R1 | 1.43 | 1.18E−03 | 3.22E−03 |
| WBP1L | 1.43 | 1.41E−04 | 6.82E−04 |
| ATL3 | 1.43 | 3.81E−04 | 1.38E−03 |
| INO80C | 1.43 | 4.91E−03 | 9.87E−03 |
| PELI1 | 1.43 | 1.77E−03 | 4.42E−03 |
| LMF2 | 1.43 | 1.05E−03 | 2.95E−03 |
| PPP2R1B | 1.43 | 5.77E−04 | 1.88E−03 |
| ENO2 | 1.43 | 3.72E−04 | 1.36E−03 |
| TPM4 | 1.43 | 1.30E−06 | 4.29E−05 |
| DOK4 | 1.42 | 2.87E−03 | 6.49E−03 |
| KRT15 | 1.42 | 1.41E−03 | 3.69E−03 |
| STIM2 | 1.42 | 2.69E−04 | 1.07E−03 |
| MLXIP | 1.42 | 1.09E−04 | 5.74E−04 |
| DBN1 | 1.42 | 6.84E−05 | 4.19E−04 |
| ADO | 1.42 | 1.37E−03 | 3.62E−03 |
| CUEDC1 | 1.42 | 2.87E−06 | 6.19E−05 |
| DNAJB2 | 1.42 | 2.19E−04 | 9.19E−04 |
| ETV3 | 1.41 | 2.72E−04 | 1.07E−03 |
| CYBA | 1.41 | 3.35E−06 | 6.68E−05 |
| OSBPL3 | 1.41 | 4.12E−05 | 2.97E−04 |
| MESDC1 | 1.41 | 1.78E−03 | 4.42E−03 |
| FKBP10 | 1.41 | 4.18E−06 | 7.69E−05 |
| SGK1 | 1.41 | 1.59E−04 | 7.39E−04 |
| CYLD | 1.41 | 6.99E−04 | 2.17E−03 |
| FRYL | 1.41 | 1.34E−04 | 6.57E−04 |
| GAK | 1.41 | 1.16E−04 | 5.91E−04 |
| FBN1 | 1.41 | 9.60E−05 | 5.29E−04 |
| TLCD1 | 1.41 | 2.12E−03 | 5.09E−03 |
| SLC22A18 | 1.40 | 1.82E−05 | 1.77E−04 |
| MAN2B1 | 1.40 | 1.13E−04 | 5.83E−04 |
| NT5DC3 | 1.40 | 3.13E−03 | 6.96E−03 |
| NFIL3 | 1.40 | 3.60E−04 | 1.33E−03 |
| SLC35F2 | 1.40 | 1.38E−03 | 3.63E−03 |
| ARRDC2 | 1.40 | 8.45E−05 | 4.83E−04 |
| MXD4 | 1.40 | 2.08E−03 | 5.01E−03 |
| LAMC1 | 1.40 | 3.10E−07 | 2.21E−05 |
| ERCC5 | 1.40 | 6.45E−04 | 2.05E−03 |
| KIF16B | 1.40 | 5.97E−04 | 1.93E−03 |
| TRIM3 | 1.40 | 2.41E−03 | 5.64E−03 |
| TPRG1L | 1.40 | 4.27E−03 | 8.86E−03 |
| TLE3 | 1.40 | 8.28E−05 | 4.75E−04 |
| SNX29 | 1.40 | 6.83E−05 | 4.18E−04 |
| CALD1 | 1.40 | 1.12E−04 | 5.82E−04 |
| RELA | 1.40 | 9.06E−05 | 5.08E−04 |
| ELOVL1 | 1.39 | 1.60E−05 | 1.67E−04 |
| SALL1 | 1.39 | 4.22E−05 | 3.01E−04 |
| SYVN1 | 1.39 | 1.01E−04 | 5.48E−04 |
| SNAPC2 | 1.39 | 2.06E−03 | 4.97E−03 |
| NOL3 | 1.39 | 2.06E−04 | 8.82E−04 |
| OSTM1 | 1.39 | 2.73E−05 | 2.29E−04 |
| TMCO3 | 1.39 | 3.28E−05 | 2.58E−04 |
| RRM2B | 1.39 | 5.61E−05 | 3.65E−04 |
| TLE1 | 1.39 | 2.74E−04 | 1.08E−03 |
| PDE4DIP | 1.39 | 2.19E−03 | 5.21E−03 |
| PIKFYVE | 1.39 | 1.38E−04 | 6.72E−04 |
| FLOT2 | 1.39 | 1.15E−04 | 5.89E−04 |
| EYA3 | 1.39 | 2.38E−05 | 2.09E−04 |
| SPATA20 | 1.39 | 2.30E−03 | 5.43E−03 |
| PLP2 | 1.39 | 8.13E−06 | 1.14E−04 |
| AGA | 1.38 | 2.00E−04 | 8.61E−04 |
| MARVELD1 | 1.38 | 7.65E−05 | 4.50E−04 |
| TTC17 | 1.38 | 1.95E−04 | 8.49E−04 |
| ATG12 | 1.38 | 1.96E−04 | 8.52E−04 |
| TMEM87A | 1.38 | 1.10E−04 | 5.76E−04 |
| FRMD8 | 1.38 | 3.56E−05 | 2.71E−04 |
| DLX1 | 1.38 | 2.79E−04 | 1.09E−03 |
| ZBED1 | 1.38 | 7.25E−04 | 2.22E−03 |
| ADCY7 | 1.38 | 5.65E−04 | 1.85E−03 |
| RCAN3 | 1.38 | 1.62E−03 | 4.13E−03 |
| LRP1 | 1.37 | 3.88E−04 | 1.40E−03 |
| OBFC1 | 1.37 | 5.91E−04 | 1.92E−03 |
| TBX3 | 1.37 | 8.97E−05 | 5.05E−04 |
| C7orf43 | 1.37 | 1.03E−04 | 5.54E−04 |
| UBE2R2 | 1.37 | 5.99E−06 | 9.38E−05 |
| FGFRL1 | 1.37 | 1.68E−05 | 1.70E−04 |
| FN1 | 1.37 | 7.50E−07 | 3.10E−05 |
| STAT6 | 1.37 | 1.13E−05 | 1.38E−04 |
| PRKCD | 1.37 | 1.35E−04 | 6.61E−04 |
| PMP22 | 1.37 | 4.12E−06 | 7.62E−05 |
| SSR4 | 1.37 | 4.09E−04 | 1.46E−03 |
| ANKRD13A | 1.36 | 6.04E−05 | 3.84E−04 |
| SLC11A2 | 1.36 | 1.71E−04 | 7.77E−04 |
| IRS2 | 1.36 | 9.63E−05 | 5.29E−04 |
| FAM102A | 1.36 | 2.69E−04 | 1.07E−03 |
| DOCK7 | 1.36 | 1.74E−03 | 4.37E−03 |
| CSGALNACT2 | 1.36 | 3.34E−03 | 7.31E−03 |
| CDC42SE2 | 1.36 | 3.20E−04 | 1.21E−03 |
| SLC9B2 | 1.36 | 1.05E−03 | 2.95E−03 |
| GRAMD1A | 1.36 | 3.38E−04 | 1.26E−03 |
| FAM134A | 1.36 | 1.56E−04 | 7.33E−04 |
| TUFT1 | 1.36 | 1.11E−05 | 1.36E−04 |
| USP12 | 1.36 | 3.34E−04 | 1.25E−03 |
| FAM213A | 1.36 | 3.02E−03 | 6.74E−03 |
| GNAI2 | 1.36 | 1.65E−04 | 7.60E−04 |
| NDUFB8 | 1.36 | 5.87E−05 | 3.76E−04 |
| CERK | 1.36 | 1.60E−05 | 1.67E−04 |
| PSMD2 | 1.36 | 3.17E−05 | 2.54E−04 |
| GRIK2 | 1.35 | 2.90E−04 | 1.13E−03 |
| KYNU | 1.35 | 5.37E−05 | 3.54E−04 |
| AGPAT2 | 1.35 | 1.67E−04 | 7.67E−04 |
| GUCD1 | 1.35 | 2.13E−04 | 9.00E−04 |
| KLHL2 | 1.35 | 2.51E−04 | 1.02E−03 |
| ACTG1 | 1.35 | 1.78E−06 | 4.85E−05 |
| CTTNBP2NL | 1.35 | 4.43E−03 | 9.12E−03 |
| WDR45 | 1.35 | 1.05E−05 | 1.31E−04 |
| PLPP6 | 1.35 | 3.48E−03 | 7.54E−03 |
| PLEKHM1 | 1.35 | 1.71E−04 | 7.78E−04 |
| GLTP | 1.35 | 2.78E−04 | 1.09E−03 |
| ANXA4 | 1.35 | 6.98E−04 | 2.17E−03 |
| LOC100507377 | 1.35 | 1.06E−03 | 2.97E−03 |
| HMGCL | 1.35 | 2.34E−03 | 5.49E−03 |
| IRF1 | 1.35 | 3.17E−03 | 7.03E−03 |
| STX7 | 1.35 | 3.79E−03 | 8.06E−03 |
| VPS13C | 1.35 | 1.49E−04 | 7.09E−04 |
| PLK3 | 1.35 | 8.01E−04 | 2.39E−03 |
| ABCC10 | 1.35 | 2.76E−03 | 6.28E−03 |
| CDS2 | 1.34 | 5.13E−04 | 1.73E−03 |
| EMP3 | 1.34 | 3.13E−05 | 2.52E−04 |
| OSBPL8 | 1.34 | 2.33E−05 | 2.06E−04 |
| ARAP1 | 1.34 | 1.02E−04 | 5.51E−04 |
| IKBKE | 1.34 | 7.17E−04 | 2.21E−03 |
| ZFAND1 | 1.34 | 7.25E−04 | 2.22E−03 |
| CAPG | 1.34 | 2.18E−05 | 1.98E−04 |
| ARHGAP1 | 1.34 | 5.93E−04 | 1.92E−03 |
| WASF3 | 1.34 | 5.43E−04 | 1.80E−03 |
| SIK3 | 1.34 | 1.48E−03 | 3.85E−03 |
| HGSNAT | 1.34 | 1.59E−04 | 7.40E−04 |
| ITGB1 | 1.34 | 5.24E−06 | 8.68E−05 |
| SH3BGRL3 | 1.34 | 1.95E−04 | 8.49E−04 |
| PLPP5 | 1.34 | 1.00E−03 | 2.85E−03 |
| MINPP1 | 1.34 | 1.18E−05 | 1.42E−04 |
| RAB6A | 1.34 | 1.25E−04 | 6.27E−04 |
| CPPED1 | 1.34 | 5.18E−04 | 1.74E−03 |
| ZCCHC6 | 1.33 | 8.62E−04 | 2.52E−03 |
| NR3C1 | 1.33 | 7.35E−04 | 2.24E−03 |
| RAB18 | 1.33 | 1.57E−03 | 4.05E−03 |
| SLC48A1 | 1.33 | 3.30E−04 | 1.24E−03 |
| SERINC1 | 1.33 | 1.89E−04 | 8.33E−04 |
| COG3 | 1.33 | 2.09E−03 | 5.03E−03 |
| AP3S1 | 1.33 | 3.90E−04 | 1.40E−03 |
| RGS16 | 1.33 | 3.22E−03 | 7.11E−03 |
| FOXK1 | 1.33 | 5.08E−04 | 1.72E−03 |
| CD96 | 1.33 | 7.11E−05 | 4.29E−04 |
| VIMP | 1.33 | 2.16E−03 | 5.17E−03 |
| CSRP1 | 1.33 | 1.27E−04 | 6.34E−04 |
| FEM1C | 1.33 | 9.62E−05 | 5.29E−04 |
| CAPN10 | 1.33 | 2.82E−03 | 6.39E−03 |
| SMAGP | 1.33 | 2.87E−04 | 1.12E−03 |
| TRIM4 | 1.33 | 3.36E−03 | 7.35E−03 |
| GASS | 1.33 | 2.46E−04 | 1.00E−03 |
| ELK4 | 1.33 | 1.40E−03 | 3.66E−03 |
| OSGIN2 | 1.33 | 3.00E−04 | 1.15E−03 |
| TSPAN3 | 1.33 | 1.06E−03 | 2.97E−03 |
| LOC652276 | 1.32 | 4.82E−03 | 9.72E−03 |
| CDIPT | 1.32 | 5.39E−05 | 3.55E−04 |
| KDELC1 | 1.32 | 1.45E−03 | 3.78E−03 |
| RASSF3 | 1.32 | 4.20E−05 | 3.00E−04 |
| EPN2 | 1.32 | 1.00E−03 | 2.85E−03 |
| EFCAB14 | 1.32 | 4.76E−04 | 1.64E−03 |
| FXYD5 | 1.32 | 4.78E−05 | 3.27E−04 |
| NR1D2 | 1.32 | 1.66E−04 | 7.64E−04 |
| UGGT2 | 1.32 | 1.98E−04 | 8.56E−04 |
| SELK | 1.32 | 2.98E−03 | 6.67E−03 |
| BCAP31 | 1.32 | 2.42E−05 | 2.10E−04 |
| PARP4 | 1.32 | 3.25E−06 | 6.59E−05 |
| CERS2 | 1.32 | 1.45E−05 | 1.58E−04 |
| PIGM | 1.32 | 2.69E−04 | 1.07E−03 |
| PPP2R5B | 1.32 | 1.07E−03 | 3.00E−03 |
| MYL12A | 1.32 | 1.65E−04 | 7.62E−04 |
| RNASEK | 1.32 | 1.24E−03 | 3.35E−03 |
| 10-Sep | 1.32 | 9.61E−05 | 5.29E−04 |
| TMEM120A | 1.31 | 1.74E−04 | 7.85E−04 |
| MOAP1 | 1.31 | 2.94E−05 | 2.42E−04 |
| PCBP4 | 1.31 | 2.53E−05 | 2.17E−04 |
| CYTH2 | 1.31 | 9.86E−05 | 5.37E−04 |
| SPATA13 | 1.31 | 4.43E−04 | 1.55E−03 |
| SAR1B | 1.31 | 2.25E−04 | 9.36E−04 |
| MFSD1 | 1.31 | 1.69E−04 | 7.71E−04 |
| SNX30 | 1.31 | 1.55E−03 | 4.00E−03 |
| LOC100506548 | 1.31 | 1.63E−03 | 4.15E−03 |
| POPDC3 | 1.31 | 8.67E−04 | 2.53E−03 |
| MCOLN1 | 1.31 | 2.74E−04 | 1.08E−03 |
| GNB4 | 1.31 | 4.15E−03 | 8.67E−03 |
| PICALM | 1.31 | 8.78E−05 | 4.96E−04 |
| ALDH1A3 | 1.31 | 1.23E−06 | 4.17E−05 |
| VAMP3 | 1.30 | 2.65E−04 | 1.06E−03 |
| CAPN2 | 1.30 | 8.71E−06 | 1.18E−04 |
| UFC1 | 1.30 | 7.37E−04 | 2.25E−03 |
| ITGAV | 1.30 | 3.44E−04 | 1.28E−03 |
| ZKSCAN1 | 1.30 | 1.21E−05 | 1.43E−04 |
| GIPC1 | 1.30 | 2.79E−06 | 6.13E−05 |
| HTR7 | 1.30 | 2.17E−03 | 5.18E−03 |
| DNTTIP1 | 1.30 | 4.51E−04 | 1.57E−03 |
| ZADH2 | 1.30 | 2.67E−03 | 6.10E−03 |
| GMIP | 1.30 | 1.75E−03 | 4.38E−03 |
| PGK1 | 1.30 | 8.21E−06 | 1.14E−04 |
| TRAPPC1 | 1.30 | 4.91E−05 | 3.32E−04 |
| PHLDB1 | 1.30 | 4.86E−04 | 1.67E−03 |
| PPIC | 1.30 | 3.92E−03 | 8.29E−03 |
| SOX10 | 1.30 | 7.15E−05 | 4.30E−04 |
| DUSP7 | 1.30 | 1.04E−04 | 5.60E−04 |
| PLEKHA3 | 1.30 | 1.88E−03 | 4.63E−03 |
| JRKL | 1.30 | 4.29E−03 | 8.89E−03 |
| MLLT11 | 1.30 | 3.41E−04 | 1.27E−03 |
| CD99 | 1.30 | 2.02E−05 | 1.89E−04 |
| TMEM64 | 1.30 | 9.57E−04 | 2.74E−03 |
| H6PD | 1.29 | 9.29E−05 | 5.16E−04 |
| CSNK1E | 1.29 | 3.52E−05 | 2.70E−04 |
| TMEM63B | 1.29 | 1.12E−03 | 3.09E−03 |
| TMEM127 | 1.29 | 8.51E−04 | 2.50E−03 |
| MACF1 | 1.29 | 1.27E−05 | 1.48E−04 |
| PLPPR2 | 1.29 | 7.07E−04 | 2.19E−03 |
| SNX8 | 1.29 | 5.71E−05 | 3.70E−04 |
| EBLN3 | 1.29 | 2.41E−05 | 2.10E−04 |
| FAM3C | 1.29 | 7.95E−06 | 1.13E−04 |
| RAPGEF1 | 1.29 | 5.94E−04 | 1.92E−03 |
| VEGFB | 1.29 | 6.17E−05 | 3.89E−04 |
| REST | 1.29 | 1.25E−04 | 6.28E−04 |
| SLCO4A1 | 1.29 | 1.66E−03 | 4.22E−03 |
| DAB2IP | 1.29 | 5.72E−04 | 1.87E−03 |
| B2M | 1.29 | 1.12E−04 | 5.82E−04 |
| LIMS1 | 1.29 | 7.50E−05 | 4.44E−04 |
| ACBD5 | 1.29 | 1.91E−03 | 4.69E−03 |
| DNAJC10 | 1.29 | 6.10E−04 | 1.97E−03 |
| TMCO4 | 1.29 | 4.25E−03 | 8.84E−03 |
| GOLT1B | 1.29 | 8.60E−04 | 2.52E−03 |
| TCP11L1 | 1.29 | 3.00E−04 | 1.15E−03 |
| GUK1 | 1.28 | 3.02E−04 | 1.16E−03 |
| IMPDH1 | 1.28 | 8.48E−05 | 4.84E−04 |
| TATDN2 | 1.28 | 8.17E−05 | 4.71E−04 |
| FKBP1A | 1.28 | 4.47E−06 | 7.96E−05 |
| NOL9 | 1.28 | 8.35E−04 | 2.46E−03 |
| DNAJC4 | 1.28 | 8.07E−04 | 2.40E−03 |
| TESK1 | 1.28 | 2.40E−03 | 5.61E−03 |
| CANT1 | 1.28 | 5.10E−04 | 1.72E−03 |
| PRCP | 1.28 | 1.35E−03 | 3.58E−03 |
| KLHL42 | 1.28 | 3.27E−03 | 7.18E−03 |
| TMUB2 | 1.28 | 5.29E−04 | 1.77E−03 |
| KDSR | 1.28 | 2.93E−04 | 1.13E−03 |
| RMND5B | 1.28 | 4.74E−03 | 9.58E−03 |
| MMP16 | 1.28 | 1.82E−04 | 8.08E−04 |
| GRIPAP1 | 1.28 | 7.25E−04 | 2.22E−03 |
| TAF13 | 1.28 | 4.49E−04 | 1.57E−03 |
| SAGE1 | 1.28 | 9.48E−04 | 2.72E−03 |
| UBE2Z | 1.28 | 1.63E−04 | 7.54E−04 |
| CERCAM | 1.28 | 7.78E−04 | 2.34E−03 |
| SMARCA2 | 1.28 | 1.34E−03 | 3.57E−03 |
| FAM114A1 | 1.27 | 7.46E−04 | 2.27E−03 |
| CES2 | 1.27 | 1.31E−05 | 1.50E−04 |
| KLHL36 | 1.27 | 6.37E−04 | 2.03E−03 |
| KLC2 | 1.27 | 2.60E−04 | 1.04E−03 |
| NPR2 | 1.27 | 1.75E−04 | 7.88E−04 |
| VPS9D1 | 1.27 | 8.98E−05 | 5.05E−04 |
| TUBB3 | 1.27 | 1.42E−04 | 6.82E−04 |
| CHIC2 | 1.27 | 8.14E−04 | 2.41E−03 |
| EEF2KMT | 1.27 | 1.03E−03 | 2.89E−03 |
| 2-Mar | 1.27 | 2.70E−04 | 1.07E−03 |
| PLBD2 | 1.27 | 1.89E−03 | 4.64E−03 |
| MNT | 1.27 | 6.46E−04 | 2.05E−03 |
| TGIF1 | 1.27 | 3.54E−03 | 7.64E−03 |
| ARPC1A | 1.27 | 8.29E−06 | 1.15E−04 |
| GPN1 | 1.27 | 1.19E−03 | 3.24E−03 |
| CHMP5 | 1.27 | 6.19E−04 | 1.99E−03 |
| WDTC1 | 1.27 | 2.21E−04 | 9.26E−04 |
| XIAP | 1.27 | 4.80E−05 | 3.27E−04 |
| SQSTM1 | 1.27 | 5.69E−05 | 3.69E−04 |
| ASNS | 1.27 | 1.98E−03 | 4.82E−03 |
| ARNT | 1.27 | 3.03E−03 | 6.76E−03 |
| KIAA0754 | 1.27 | 3.52E−04 | 1.30E−03 |
| YIPF5 | 1.27 | 9.98E−04 | 2.84E−03 |
| MAPKAPK2 | 1.27 | 8.84E−04 | 2.57E−03 |
| PRDX5 | 1.27 | 7.20E−04 | 2.21E−03 |
| GBA | 1.27 | 2.97E−04 | 1.14E−03 |
| PPP1CB | 1.26 | 2.25E−04 | 9.35E−04 |
| ZBTB4 | 1.26 | 8.29E−04 | 2.45E−03 |
| GPAT3 | 1.26 | 1.72E−04 | 7.79E−04 |
| STXBP3 | 1.26 | 1.50E−03 | 3.90E−03 |
| TSHZ1 | 1.26 | 1.08E−03 | 3.00E−03 |
| AMACR | 1.26 | 3.97E−03 | 8.38E−03 |
| SMG7 | 1.26 | 1.68E−04 | 7.71E−04 |
| TCAF1 | 1.26 | 4.73E−05 | 3.26E−04 |
| TIPARP | 1.26 | 8.23E−04 | 2.44E−03 |
| HIPK1 | 1.26 | 2.91E−04 | 1.13E−03 |
| CSTB | 1.26 | 1.43E−03 | 3.75E−03 |
| TACC1 | 1.26 | 1.58E−04 | 7.37E−04 |
| MGAT4B | 1.26 | 1.87E−04 | 8.26E−04 |
| DEAF1 | 1.26 | 2.97E−03 | 6.65E−03 |
| YAP1 | 1.26 | 8.68E−04 | 2.53E−03 |
| PLOD1 | 1.26 | 1.06E−04 | 5.64E−04 |
| SUCO | 1.26 | 4.61E−04 | 1.60E−03 |
| TM9SF3 | 1.26 | 7.83E−04 | 2.35E−03 |
| POLG | 1.26 | 3.37E−03 | 7.36E−03 |
| TRIM56 | 1.25 | 4.56E−04 | 1.59E−03 |
| PMAIP1 | 1.25 | 2.16E−03 | 5.16E−03 |
| AGFG1 | 1.25 | 5.45E−04 | 1.81E−03 |
| EMP1 | 1.25 | 4.92E−04 | 1.68E−03 |
| RPS4X | 1.25 | 1.65E−07 | 1.75E−05 |
| ZC3HAV1 | 1.25 | 6.90E−04 | 2.15E−03 |
| SCYL2 | 1.25 | 1.31E−05 | 1.49E−04 |
| TDRKH | 1.25 | 4.21E−03 | 8.77E−03 |
| B4GALT7 | 1.25 | 2.28E−03 | 5.40E−03 |
| FAM214A | 1.25 | 1.39E−03 | 3.66E−03 |
| PTBP3 | 1.25 | 3.45E−04 | 1.28E−03 |
| RHOG | 1.25 | 3.36E−03 | 7.35E−03 |
| PBX3 | 1.25 | 1.60E−04 | 7.44E−04 |
| LAMB2 | 1.25 | 8.97E−04 | 2.60E−03 |
| PACS1 | 1.25 | 3.45E−04 | 1.28E−03 |
| ARHGEF12 | 1.25 | 4.03E−05 | 2.92E−04 |
| ULK3 | 1.25 | 3.26E−03 | 7.17E−03 |
| POGK | 1.25 | 1.01E−03 | 2.87E−03 |
| MTRNR2L8 | 1.25 | 2.58E−03 | 5.94E−03 |
| HPS6 | 1.25 | 2.06E−03 | 4.98E−03 |
| SLK | 1.25 | 6.45E−04 | 2.05E−03 |
| NUDT22 | 1.25 | 1.77E−03 | 4.42E−03 |
| RNF146 | 1.25 | 4.26E−03 | 8.84E−03 |
| LRRC8A | 1.25 | 5.87E−05 | 3.76E−04 |
| CHMP3 | 1.25 | 9.78E−04 | 2.79E−03 |
| ATP6V1G1 | 1.25 | 6.07E−05 | 3.86E−04 |
| UTRN | 1.25 | 2.80E−03 | 6.34E−03 |
| EXOC7 | 1.24 | 2.74E−04 | 1.08E−03 |
| LOC102723724 | 1.24 | 7.02E−04 | 2.18E−03 |
| TMEM50A | 1.24 | 3.46E−03 | 7.50E−03 |
| PLOD3 | 1.24 | 5.40E−06 | 8.78E−05 |
| LINC00467 | 1.24 | 1.53E−04 | 7.23E−04 |
| HERC4 | 1.24 | 3.61E−03 | 7.75E−03 |
| SBF1 | 1.24 | 2.85E−04 | 1.11E−03 |
| SMIM19 | 1.24 | 1.32E−03 | 3.52E−03 |
| C16orf45 | 1.24 | 6.75E−04 | 2.12E−03 |
| ARPC5 | 1.24 | 5.94E−05 | 3.80E−04 |
| HS3ST3A1 | 1.24 | 1.35E−03 | 3.58E−03 |
| MGST1 | 1.24 | 6.59E−04 | 2.08E−03 |
| MORC3 | 1.24 | 4.11E−03 | 8.60E−03 |
| CREB3 | 1.24 | 4.16E−03 | 8.69E−03 |
| ITPKC | 1.24 | 5.60E−04 | 1.84E−03 |
| SHARPIN | 1.24 | 1.01E−03 | 2.86E−03 |
| ATP6AP2 | 1.24 | 2.08E−04 | 8.89E−04 |
| CTSB | 1.24 | 1.22E−04 | 6.16E−04 |
| NOTCH2 | 1.24 | 1.72E−05 | 1.72E−04 |
| CCDC93 | 1.24 | 2.75E−04 | 1.08E−03 |
| NIPA1 | 1.24 | 6.67E−05 | 4.11E−04 |
| PTDSS1 | 1.24 | 1.19E−05 | 1.42E−04 |
| BICD1 | 1.24 | 1.68E−03 | 4.24E−03 |
| CMAS | 1.24 | 1.45E−03 | 3.78E−03 |
| ALDH9A1 | 1.24 | 1.27E−04 | 6.34E−04 |
| DPP3 | 1.24 | 7.80E−05 | 4.57E−04 |
| ATG101 | 1.24 | 2.30E−06 | 5.55E−05 |
| G3BP1 | 1.23 | 2.96E−06 | 6.25E−05 |
| NECAP2 | 1.23 | 1.12E−03 | 3.09E−03 |
| GPR107 | 1.23 | 3.72E−04 | 1.36E−03 |
| CCND1 | 1.23 | 3.05E−06 | 6.38E−05 |
| GRINA | 1.23 | 1.44E−04 | 6.89E−04 |
| ATXN10 | 1.23 | 5.36E−05 | 3.53E−04 |
| UBR1 | 1.23 | 2.68E−03 | 6.13E−03 |
| ZBTB7A | 1.23 | 3.54E−03 | 7.63E−03 |
| GNG10 | 1.23 | 3.73E−04 | 1.36E−03 |
| MC1R | 1.23 | 4.76E−03 | 9.61E−03 |
| NDFIP1 | 1.23 | 1.30E−04 | 6.42E−04 |
| SLMAP | 1.23 | 2.25E−03 | 5.33E−03 |
| DUSP23 | 1.23 | 1.66E−03 | 4.20E−03 |
| C1orf122 | 1.23 | 4.10E−03 | 8.59E−03 |
| RASAL2 | 1.23 | 1.32E−04 | 6.50E−04 |
| TOLLIP | 1.23 | 3.46E−03 | 7.50E−03 |
| HECTD3 | 1.23 | 3.18E−04 | 1.20E−03 |
| MPP6 | 1.23 | 5.45E−04 | 1.81E−03 |
| UBE2L6 | 1.23 | 1.84E−03 | 4.53E−03 |
| SESN2 | 1.23 | 1.84E−03 | 4.54E−03 |
| ATP6V1D | 1.22 | 2.75E−03 | 6.26E−03 |
| BRSK1 | 1.22 | 1.94E−04 | 8.46E−04 |
| APMAP | 1.22 | 5.36E−04 | 1.79E−03 |
| WDR13 | 1.22 | 1.71E−03 | 4.30E−03 |
| TMEM9B | 1.22 | 2.14E−04 | 9.04E−04 |
| FBXO17 | 1.22 | 3.84E−03 | 8.15E−03 |
| FSCN1 | 1.22 | 7.14E−05 | 4.30E−04 |
| B3GAT3 | 1.22 | 1.15E−03 | 3.15E−03 |
| CTTN | 1.22 | 2.30E−04 | 9.50E−04 |
| INSIG1 | 1.22 | 1.39E−03 | 3.66E−03 |
| CORO1B | 1.22 | 1.36E−04 | 6.66E−04 |
| MYO9B | 1.22 | 1.46E−05 | 1.58E−04 |
| CBFB | 1.22 | 7.33E−04 | 2.24E−03 |
| KLHDC2 | 1.22 | 2.36E−04 | 9.71E−04 |
| RPS12 | 1.22 | 1.15E−05 | 1.40E−04 |
| ZER1 | 1.21 | 2.46E−04 | 1.00E−03 |
| SDE2 | 1.21 | 1.01E−03 | 2.87E−03 |
| NCEH1 | 1.21 | 5.52E−04 | 1.83E−03 |
| RECQL | 1.21 | 1.50E−04 | 7.10E−04 |
| COMMD6 | 1.21 | 4.79E−03 | 9.67E−03 |
| LIX1L | 1.21 | 7.07E−04 | 2.19E−03 |
| FBXO28 | 1.21 | 9.03E−04 | 2.62E−03 |
| KDM5B | 1.21 | 9.03E−06 | 1.20E−04 |
| TMED4 | 1.21 | 4.06E−04 | 1.45E−03 |
| CMIP | 1.21 | 4.61E−03 | 9.38E−03 |
| ZNF444 | 1.21 | 1.37E−03 | 3.62E−03 |
| CTSF | 1.21 | 2.64E−03 | 6.06E−03 |
| TNIP2 | 1.21 | 2.34E−03 | 5.49E−03 |
| JADE1 | 1.21 | 2.99E−03 | 6.67E−03 |
| CD47 | 1.21 | 1.81E−03 | 4.49E−03 |
| PLEC | 1.21 | 8.56E−04 | 2.51E−03 |
| EEF2 | 1.21 | 5.30E−05 | 3.50E−04 |
| WASL | 1.21 | 3.27E−03 | 7.18E−03 |
| MAST3 | 1.21 | 4.68E−03 | 9.49E−03 |
| ERP44 | 1.21 | 3.91E−03 | 8.28E−03 |
| PITRM1 | 1.21 | 2.90E−04 | 1.12E−03 |
| MICA | 1.21 | 4.10E−05 | 2.96E−04 |
| PLIN3 | 1.21 | 4.30E−04 | 1.51E−03 |
| FRMD4A | 1.20 | 7.57E−04 | 2.29E−03 |
| TNFRSF1A | 1.20 | 2.53E−04 | 1.02E−03 |
| PTPN9 | 1.20 | 4.92E−04 | 1.68E−03 |
| TUG1 | 1.20 | 4.69E−04 | 1.62E−03 |
| LTBP3 | 1.20 | 1.41E−04 | 6.81E−04 |
| TVP23B | 1.20 | 1.98E−04 | 8.56E−04 |
| NAPG | 1.20 | 3.26E−04 | 1.23E−03 |
| GOLGA1 | 1.20 | 4.92E−03 | 9.88E−03 |
| CD164 | 1.20 | 1.16E−03 | 3.18E−03 |
| FBXO11 | 1.20 | 1.29E−03 | 3.45E−03 |
| RGP1 | 1.20 | 1.62E−04 | 7.50E−04 |
| AKAP13 | 1.20 | 4.87E−05 | 3.31E−04 |
| UGDH-AS1 | 1.20 | 5.04E−04 | 1.71E−03 |
| ZPR1 | 1.20 | 6.54E−04 | 2.07E−03 |
| CAST | 1.20 | 3.73E−04 | 1.36E−03 |
| SPCS3 | 1.20 | 2.16E−03 | 5.16E−03 |
| NR1H2 | 1.20 | 1.87E−04 | 8.26E−04 |
| RRBP1 | 1.20 | 1.63E−05 | 1.68E−04 |
| C6orf1 | 1.20 | 8.56E−04 | 2.51E−03 |
| IGBP1 | 1.20 | 4.01E−03 | 8.43E−03 |
| REEP5 | 1.20 | 9.70E−04 | 2.77E−03 |
| FAH | 1.20 | 2.78E−03 | 6.31E−03 |
| PDPK1 | 1.19 | 1.47E−03 | 3.82E−03 |
| MOV10 | 1.19 | 3.18E−03 | 7.05E−03 |
| SLC20A1 | 1.19 | 1.08E−04 | 5.68E−04 |
| MSL2 | 1.19 | 9.27E−05 | 5.15E−04 |
| IKBKB | 1.19 | 4.35E−03 | 8.98E−03 |
| NSMAF | 1.19 | 7.28E−04 | 2.23E−03 |
| ARHGEF1 | 1.19 | 2.13E−04 | 9.01E−04 |
| TMEM59 | 1.19 | 2.83E−03 | 6.39E−03 |
| SPRED2 | 1.19 | 2.54E−03 | 5.88E−03 |
| GNB1 | 1.19 | 7.93E−05 | 4.61E−04 |
| KBTBD2 | 1.19 | 2.78E−03 | 6.31E−03 |
| FAT1 | 1.19 | 2.55E−04 | 1.03E−03 |
| MEX3C | 1.19 | 1.26E−03 | 3.40E−03 |
| ERAP1 | 1.19 | 2.77E−03 | 6.29E−03 |
| CACYBP | 1.19 | 2.91E−04 | 1.13E−03 |
| AP1B1 | 1.19 | 9.55E−05 | 5.27E−04 |
| FAM222B | 1.18 | 9.00E−04 | 2.61E−03 |
| VIM | 1.18 | 9.83E−05 | 5.37E−04 |
| EXOC2 | 1.18 | 1.90E−03 | 4.67E−03 |
| PTTG1IP | 1.18 | 1.34E−03 | 3.57E−03 |
| TERF2IP | 1.18 | 1.17E−04 | 5.98E−04 |
| C6orf89 | 1.18 | 1.18E−03 | 3.23E−03 |
| CAP1 | 1.18 | 1.28E−04 | 6.36E−04 |
| NIN | 1.18 | 4.11E−03 | 8.60E−03 |
| INF2 | 1.18 | 4.42E−03 | 9.10E−03 |
| RPS6KA4 | 1.18 | 1.67E−03 | 4.24E−03 |
| RC3H2 | 1.18 | 2.25E−03 | 5.33E−03 |
| SND1 | 1.18 | 1.76E−04 | 7.92E−04 |
| HTATIP2 | 1.18 | 3.24E−03 | 7.14E−03 |
| NEK7 | 1.18 | 4.99E−04 | 1.70E−03 |
| SSR3 | 1.18 | 4.73E−05 | 3.26E−04 |
| DPY19L1 | 1.18 | 4.09E−04 | 1.46E−03 |
| FAM234A | 1.18 | 1.44E−03 | 3.77E−03 |
| TNK2 | 1.18 | 4.07E−04 | 1.45E−03 |
| SEMA7A | 1.18 | 3.77E−04 | 1.37E−03 |
| EDEM2 | 1.18 | 1.39E−03 | 3.65E−03 |
| PPM1A | 1.17 | 2.20E−03 | 5.23E−03 |
| ATP6V1A | 1.17 | 9.16E−05 | 5.11E−04 |
| ZNF707 | 1.17 | 1.05E−03 | 2.95E−03 |
| EDEM3 | 1.17 | 1.38E−03 | 3.64E−03 |
| KCTD5 | 1.17 | 5.83E−04 | 1.90E−03 |
| PDCD10 | 1.17 | 3.74E−03 | 7.98E−03 |
| TMEM208 | 1.17 | 7.01E−04 | 2.18E−03 |
| YKT6 | 1.17 | 6.69E−04 | 2.11E−03 |
| HEXA | 1.17 | 4.82E−03 | 9.72E−03 |
| DYRK3 | 1.17 | 1.54E−03 | 3.97E−03 |
| ARPC5L | 1.17 | 5.65E−05 | 3.67E−04 |
| DRAM1 | 1.17 | 4.92E−03 | 9.87E−03 |
| ERLEC1 | 1.17 | 2.27E−03 | 5.37E−03 |
| TBCEL | 1.17 | 2.40E−03 | 5.60E−03 |
| UBA1 | 1.17 | 2.74E−06 | 6.08E−05 |
| ARF4 | 1.17 | 8.77E−04 | 2.56E−03 |
| LCMT2 | 1.17 | 4.45E−03 | 9.14E−03 |
| STK4 | 1.17 | 3.77E−03 | 8.02E−03 |
| SLC35E1 | 1.17 | 3.64E−04 | 1.34E−03 |
| YIF1A | 1.17 | 2.44E−03 | 5.69E−03 |
| RPLP1 | 1.17 | 2.34E−05 | 2.07E−04 |
| COPA | 1.17 | 3.82E−05 | 2.84E−04 |
| TNKS | 1.16 | 2.46E−03 | 5.74E−03 |
| ESRRA | 1.16 | 4.00E−03 | 8.43E−03 |
| METTL23 | 1.16 | 2.83E−03 | 6.39E−03 |
| DESI2 | 1.16 | 2.20E−04 | 9.21E−04 |
| MKRN1 | 1.16 | 1.82E−03 | 4.50E−03 |
| TMC01 | 1.16 | 4.46E−04 | 1.56E−03 |
| MIA3 | 1.16 | 3.26E−03 | 7.17E−03 |
| AP5Z1 | 1.16 | 4.78E−03 | 9.65E−03 |
| LYPLA2 | 1.16 | 5.01E−04 | 1.70E−03 |
| NUCB1 | 1.16 | 1.70E−04 | 7.75E−04 |
| IGF2R | 1.16 | 2.17E−06 | 5.42E−05 |
| CCPG1 | 1.16 | 4.96E−03 | 9.94E−03 |
| IKBIP | 1.16 | 3.49E−03 | 7.57E−03 |
| ERBIN | 1.16 | 3.87E−03 | 8.21E−03 |
| BCR | 1.16 | 2.65E−03 | 6.07E−03 |
| ZNF598 | 1.16 | 1.68E−03 | 4.25E−03 |
| AHNAK | 1.16 | 1.77E−03 | 4.41E−03 |
| SPTBN1 | 1.16 | 2.29E−04 | 9.47E−04 |
| TMBIM6 | 1.16 | 1.39E−06 | 4.43E−05 |
| CSNK1A1 | 1.16 | 5.78E−04 | 1.89E−03 |
| FLYWCH1 | 1.16 | 4.60E−03 | 9.36E−03 |
| TIMM10B | 1.16 | 3.58E−03 | 7.70E−03 |
| CEP170 | 1.16 | 7.70E−04 | 2.32E−03 |
| RAP1GDS1 | 1.16 | 4.97E−03 | 9.95E−03 |
| DALRD3 | 1.16 | 6.38E−04 | 2.03E−03 |
| ARPP19 | 1.16 | 1.40E−03 | 3.67E−03 |
| RNF13 | 1.16 | 1.04E−03 | 2.93E−03 |
| INTS7 | 1.16 | 6.79E−04 | 2.13E−03 |
| SNAP23 | 1.16 | 3.24E−03 | 7.14E−03 |
| CYB5R3 | 1.16 | 3.87E−04 | 1.40E−03 |
| MIA | 1.16 | 3.49E−03 | 7.57E−03 |
| ATP6V0B | 1.15 | 1.09E−03 | 3.02E−03 |
| NOP9 | 1.15 | 1.24E−04 | 6.24E−04 |
| SNHG6 | 1.15 | 2.33E−04 | 9.61E−04 |
| TMEM184B | 1.15 | 1.86E−03 | 4.59E−03 |
| MED15 | 1.15 | 4.57E−03 | 9.32E−03 |
| TRIM11 | 1.15 | 8.84E−04 | 2.57E−03 |
| MAEA | 1.15 | 2.08E−03 | 5.00E−03 |
| INPPL1 | 1.15 | 1.55E−05 | 1.64E−04 |
| GNB5 | 1.15 | 1.89E−04 | 8.32E−04 |
| NOMO1 | 1.15 | 1.71E−03 | 4.31E−03 |
| ATP6V0C | 1.15 | 4.40E−03 | 9.07E−03 |
| PURB | 1.15 | 5.59E−04 | 1.84E−03 |
| GNB2 | 1.15 | 3.98E−05 | 2.91E−04 |
| PCSK7 | 1.15 | 4.65E−03 | 9.44E−03 |
| WASF2 | 1.15 | 6.40E−04 | 2.04E−03 |
| SLC25A4 | 1.15 | 1.47E−03 | 3.82E−03 |
| TMED7 | 1.15 | 3.39E−05 | 2.64E−04 |
| SMARCAL1 | 1.15 | 1.78E−04 | 7.96E−04 |
| DBNL | 1.15 | 3.47E−03 | 7.53E−03 |
| KIDINS220 | 1.14 | 3.57E−03 | 7.68E−03 |
| TMEM189 | 1.14 | 1.03E−03 | 2.90E−03 |
| SEC61A1 | 1.14 | 1.28E−04 | 6.37E−04 |
| PNPLA2 | 1.14 | 9.43E−04 | 2.71E−03 |
| RPL3 | 1.14 | 2.16E−05 | 1.97E−04 |
| CDKN1B | 1.14 | 3.99E−03 | 8.42E−03 |
| FIBP | 1.14 | 1.54E−05 | 1.64E−04 |
| NFE2L1 | 1.14 | 9.82E−05 | 5.37E−04 |
| STUB1 | 1.14 | 8.96E−04 | 2.60E−03 |
| PIGT | 1.14 | 4.66E−04 | 1.62E−03 |
| YRDC | 1.14 | 5.23E−04 | 1.75E−03 |
| MICALL1 | 1.14 | 2.67E−04 | 1.06E−03 |
| TPT1 | 1.14 | 1.69E−04 | 7.73E−04 |
| PTK2 | 1.14 | 2.70E−03 | 6.16E−03 |
| ACVR1 | 1.14 | 4.60E−03 | 9.37E−03 |
| NENF | 1.14 | 1.38E−03 | 3.63E−03 |
| GPAA1 | 1.14 | 3.09E−04 | 1.18E−03 |
| RNF14 | 1.14 | 1.70E−04 | 7.74E−04 |
| COX6B1 | 1.14 | 7.09E−04 | 2.19E−03 |
| AMPD2 | 1.14 | 4.41E−03 | 9.08E−03 |
| MFSD10 | 1.14 | 3.67E−03 | 7.86E−03 |
| MAP4K4 | 1.14 | 5.30E−05 | 3.50E−04 |
| BAG1 | 1.14 | 3.91E−04 | 1.41E−03 |
| EPM2AIP1 | 1.14 | 4.56E−03 | 9.32E−03 |
| EHBP1L1 | 1.14 | 7.18E−04 | 2.21E−03 |
| RNF4 | 1.14 | 4.32E−03 | 8.93E−03 |
| CPE | 1.14 | 2.25E−04 | 9.37E−04 |
| PYGL | 1.14 | 7.14E−04 | 2.20E−03 |
| P3H4 | 1.13 | 2.27E−03 | 5.37E−03 |
| BLOC1S6 | 1.13 | 1.53E−03 | 3.96E−03 |
| TMEM259 | 1.13 | 7.14E−04 | 2.20E−03 |
| PIP4K2B | 1.13 | 2.44E−03 | 5.68E−03 |
| DNAJC5 | 1.13 | 5.20E−05 | 3.46E−04 |
| NORAD | 1.13 | 9.37E−04 | 2.70E−03 |
| ERRFI1 | 1.13 | 6.36E−04 | 2.03E−03 |
| STARD3 | 1.13 | 3.26E−03 | 7.18E−03 |
| TRIM32 | 1.13 | 4.88E−03 | 9.82E−03 |
| BET1L | 1.13 | 1.27E−03 | 3.41E−03 |
| MORF4L2 | 1.13 | 7.04E−05 | 4.26E−04 |
| ATP6V1H | 1.13 | 2.58E−03 | 5.95E−03 |
| FAM199X | 1.13 | 1.18E−03 | 3.23E−03 |
| GYG1 | 1.13 | 4.25E−03 | 8.83E−03 |
| ACTR3 | 1.13 | 1.65E−03 | 4.18E−03 |
| PRR12 | 1.13 | 1.31E−04 | 6.46E−04 |
| FAM127B | 1.13 | 2.71E−03 | 6.19E−03 |
| NCSTN | 1.13 | 1.75E−03 | 4.37E−03 |
| ECE1 | 1.12 | 4.19E−04 | 1.48E−03 |
| C12orf49 | 1.12 | 2.62E−03 | 6.02E−03 |
| MYO1E | 1.12 | 1.39E−03 | 3.66E−03 |
| C11orf24 | 1.12 | 4.65E−04 | 1.61E−03 |
| CDK5RAP2 | 1.12 | 3.40E−03 | 7.41E−03 |
| RANGAP1 | 1.12 | 8.93E−04 | 2.59E−03 |
| ACO1 | 1.12 | 8.31E−04 | 2.45E−03 |
| TMED9 | 1.12 | 2.98E−04 | 1.15E−03 |
| LRRC75A-AS1 | 1.12 | 4.15E−03 | 8.67E−03 |
| SEMA3B | 1.12 | 4.57E−03 | 9.32E−03 |
| YIPF3 | 1.12 | 1.19E−03 | 3.24E−03 |
| FLII | 1.12 | 5.06E−04 | 1.71E−03 |
| CTBP1-AS2 | 1.12 | 4.51E−03 | 9.23E−03 |
| VANGL1 | 1.12 | 3.01E−03 | 6.72E−03 |
| RPL10 | 1.12 | 1.17E−04 | 5.98E−04 |
| TFRC | 1.12 | 2.11E−03 | 5.07E−03 |
| GLB1 | 1.12 | 3.63E−03 | 7.78E−03 |
| ACOT8 | 1.11 | 2.41E−03 | 5.64E−03 |
| COPE | 1.11 | 2.31E−03 | 5.44E−03 |
| EIF2S3 | 1.11 | 3.51E−04 | 1.30E−03 |
| DPP9 | 1.11 | 1.16E−03 | 3.18E−03 |
| DCAF12 | 1.11 | 4.37E−03 | 9.02E−03 |
| ISG20 | 1.11 | 7.21E−04 | 2.21E−03 |
| CD276 | 1.11 | 3.26E−03 | 7.18E−03 |
| PTPN23 | 1.11 | 3.13E−04 | 1.19E−03 |
| PHRF1 | 1.11 | 3.30E−03 | 7.25E−03 |
| MAVS | 1.11 | 1.65E−03 | 4.18E−03 |
| ITM2B | 1.11 | 1.11E−04 | 5.78E−04 |
| TPM3 | 1.11 | 1.21E−03 | 3.28E−03 |
| LARP1 | 1.11 | 4.38E−04 | 1.54E−03 |
| HOMER1 | 1.11 | 2.60E−03 | 5.98E−03 |
| PFKP | 1.11 | 2.28E−03 | 5.38E−03 |
| G6PC3 | 1.11 | 2.44E−03 | 5.68E−03 |
| CDK16 | 1.11 | 1.98E−03 | 4.82E−03 |
| TM7SF3 | 1.11 | 3.16E−03 | 7.00E−03 |
| NUDCD3 | 1.11 | 3.93E−03 | 8.32E−03 |
| RPLPO | 1.11 | 2.50E−04 | 1.02E−03 |
| ARPC2 | 1.11 | 3.00E−03 | 6.69E−03 |
| MCFD2 | 1.11 | 4.72E−03 | 9.56E−03 |
| PRDX2 | 1.11 | 4.48E−03 | 9.19E−03 |
| FBRS | 1.11 | 5.16E−04 | 1.73E−03 |
| YIPF2 | 1.10 | 4.55E−03 | 9.30E−03 |
| CLPTM1 | 1.10 | 1.61E−03 | 4.10E−03 |
| DYNLRB1 | 1.10 | 9.11E−04 | 2.63E−03 |
| HM13 | 1.10 | 9.96E−04 | 2.84E−03 |
| PRUNE | 1.10 | 2.47E−03 | 5.75E−03 |
| CD109 | 1.10 | 2.40E−03 | 5.61E−03 |
| FBXO31 | 1.10 | 3.31E−03 | 7.27E−03 |
| CDC42 | 1.10 | 4.39E−03 | 9.05E−03 |
| SCAF11 | 1.10 | 7.70E−05 | 4.52E−04 |
| SEC23B | 1.10 | 4.60E−03 | 9.37E−03 |
| ARF1 | 1.09 | 4.16E−04 | 1.48E−03 |
| C16orf58 | 1.09 | 6.00E−04 | 1.93E−03 |
| STX16 | 1.09 | 5.53E−04 | 1.83E−03 |
| MARK2 | 1.09 | 2.11E−03 | 5.08E−03 |
| RPL39 | 1.09 | 5.03E−04 | 1.71E−03 |
| ASAP2 | 1.09 | 4.24E−03 | 8.81E−03 |
| IGF1R | 1.09 | 3.63E−03 | 7.79E−03 |
| PAFAH2 | 1.09 | 5.97E−04 | 1.93E−03 |
| URGCP | 1.09 | 2.83E−03 | 6.39E−03 |
| KMT2D | 1.09 | 8.63E−04 | 2.52E−03 |
| YWHAG | 1.09 | 2.98E−03 | 6.67E−03 |
| RERE | 1.09 | 3.43E−03 | 7.45E−03 |
| RPL13A | 1.09 | 4.42E−04 | 1.55E−03 |
| STT3A | 1.08 | 8.60E−04 | 2.52E−03 |
| ACTB | 1.08 | 1.25E−04 | 6.29E−04 |
| RPS20 | 1.08 | 4.70E−05 | 3.25E−04 |
| TRAM1 | 1.08 | 4.14E−03 | 8.66E−03 |
| EIF3F | 1.08 | 4.16E−03 | 8.69E−03 |
| ATRAID | 1.08 | 7.28E−04 | 2.23E−03 |
| CANX | 1.08 | 4.65E−05 | 3.22E−04 |
| RPS3 | 1.08 | 6.15E−04 | 1.98E−03 |
| TRPC4AP | 1.08 | 1.73E−03 | 4.34E−03 |
| HRAS | 1.07 | 3.07E−03 | 6.84E−03 |
| RPS28 | 1.07 | 7.50E−04 | 2.28E−03 |
| FAM96B | 1.07 | 4.86E−03 | 9.78E−03 |
| CASC4 | 1.07 | 3.20E−03 | 7.08E−03 |
| PABPC1 | 1.07 | 1.72E−05 | 1.72E−04 |
| ELK3 | 1.07 | 1.42E−04 | 6.82E−04 |
| CDC37 | 1.07 | 1.08E−03 | 3.00E−03 |
| CD63 | 1.06 | 3.53E−03 | 7.63E−03 |
| JAK1 | 1.06 | 3.40E−03 | 7.41E−03 |
| MTAP | 1.06 | 1.78E−03 | 4.43E−03 |
| UGGT1 | 1.06 | 1.79E−03 | 4.45E−03 |
| CHTF8 | 1.06 | 1.80E−03 | 4.47E−03 |
| TGOLN2 | 1.05 | 1.73E−03 | 4.35E−03 |
| PEA15 | 1.05 | 2.16E−03 | 5.16E−03 |
| PPIB | 1.05 | 3.43E−03 | 7.45E−03 |
| P4HB | 1.05 | 1.92E−03 | 4.71E−03 |
| ATG13 | 1.05 | 1.60E−03 | 4.09E−03 |
| SLC6A8 | 1.05 | 1.73E−03 | 4.34E−03 |
| NRAS | 1.04 | 4.03E−03 | 8.47E−03 |
| STK25 | 1.04 | 4.89E−03 | 9.83E−03 |
| RPL17 | 0.96 | 2.46E−03 | 5.74E−03 |
| RGL2 | 0.96 | 3.76E−03 | 8.01E−03 |
| NDUFB11 | 0.96 | 3.07E−03 | 6.83E−03 |
| EIF4A1 | 0.96 | 5.09E−04 | 1.72E−03 |
| HMGXB3 | 0.96 | 9.41E−05 | 5.21E−04 |
| MCAM | 0.95 | 5.65E−04 | 1.85E−03 |
| ATP2A2 | 0.95 | 1.00E−03 | 2.85E−03 |
| RPL4 | 0.95 | 3.19E−04 | 1.20E−03 |
| RPL7 | 0.95 | 2.38E−03 | 5.57E−03 |
| RPSA | 0.95 | 4.45E−03 | 9.14E−03 |
| EIF3A | 0.95 | 3.88E−03 | 8.22E−03 |
| LDOCIL | 0.95 | 4.18E−03 | 8.71E−03 |
| PSMD8 | 0.95 | 4.26E−03 | 8.85E−03 |
| LETM1 | 0.95 | 4.48E−03 | 9.19E−03 |
| GAPDH | 0.95 | 1.12E−03 | 3.09E−03 |
| EIF3G | 0.95 | 2.92E−03 | 6.56E−03 |
| FAU | 0.94 | 3.64E−03 | 7.80E−03 |
| PFN1 | 0.94 | 4.79E−04 | 1.65E−03 |
| ATP5H | 0.94 | 2.70E−03 | 6.16E−03 |
| AHSA1 | 0.94 | 2.10E−04 | 8.95E−04 |
| DDX23 | 0.94 | 2.85E−03 | 6.44E−03 |
| 2-Sep | 0.94 | 1.32E−03 | 3.51E−03 |
| RPS7 | 0.94 | 8.49E−04 | 2.49E−03 |
| SLC25A3 | 0.94 | 6.46E−04 | 2.05E−03 |
| EIF4H | 0.94 | 1.34E−04 | 6.56E−04 |
| EIF1AX | 0.94 | 4.83E−03 | 9.74E−03 |
| COPS6 | 0.93 | 2.43E−03 | 5.67E−03 |
| PRKCI | 0.93 | 1.46E−03 | 3.81E−03 |
| TAF7 | 0.93 | 1.29E−03 | 3.45E−03 |
| B4GALT5 | 0.93 | 4.40E−03 | 9.07E−03 |
| PAIP1 | 0.93 | 2.12E−03 | 5.09E−03 |
| RPL18 | 0.93 | 3.64E−03 | 7.80E−03 |
| ACTRIA | 0.93 | 2.57E−03 | 5.93E−03 |
| COPS8 | 0.93 | 2.91E−04 | 1.13E−03 |
| HINT1 | 0.93 | 4.98E−05 | 3.35E−04 |
| DCTN1 | 0.93 | 1.81E−03 | 4.49E−03 |
| DIAPH1 | 0.93 | 3.67E−03 | 7.85E−03 |
| ZMYM4 | 0.93 | 1.18E−03 | 3.22E−03 |
| LRRC59 | 0.93 | 3.35E−05 | 2.63E−04 |
| GNPAT | 0.93 | 1.08E−03 | 3.01E−03 |
| C6orf106 | 0.93 | 1.04E−03 | 2.93E−03 |
| BCAR1 | 0.93 | 3.49E−04 | 1.29E−03 |
| CHERP | 0.93 | 2.74E−03 | 6.24E−03 |
| EIF5 | 0.93 | 4.62E−03 | 9.40E−03 |
| RPS24 | 0.93 | 1.61E−04 | 7.48E−04 |
| LDHB | 0.93 | 2.64E−03 | 6.06E−03 |
| C11orf58 | 0.93 | 3.56E−03 | 7.66E−03 |
| ARFGAP1 | 0.92 | 1.37E−04 | 6.68E−04 |
| RETSAT | 0.92 | 2.12E−03 | 5.09E−03 |
| DTD1 | 0.92 | 3.44E−04 | 1.28E−03 |
| PDIA3 | 0.92 | 1.42E−03 | 3.72E−03 |
| RPL30 | 0.92 | 3.36E−04 | 1.26E−03 |
| TMEM230 | 0.92 | 1.00E−03 | 2.85E−03 |
| CD74 | 0.92 | 3.99E−05 | 2.91E−04 |
| CTDNEP1 | 0.92 | 4.42E−03 | 9.09E−03 |
| HDLBP | 0.92 | 6.99E−05 | 4.25E−04 |
| PDIA6 | 0.92 | 9.91E−04 | 2.82E−03 |
| GTF3C1 | 0.92 | 3.72E−04 | 1.36E−03 |
| CLOCK | 0.92 | 1.44E−03 | 3.76E−03 |
| BRD2 | 0.92 | 2.78E−03 | 6.32E−03 |
| RNH1 | 0.92 | 4.54E−03 | 9.29E−03 |
| KAT6A | 0.92 | 4.78E−03 | 9.65E−03 |
| CSDE1 | 0.92 | 1.87E−03 | 4.60E−03 |
| ACP2 | 0.92 | 3.73E−04 | 1.36E−03 |
| PRPF6 | 0.92 | 1.79E−04 | 8.00E−04 |
| CCDC97 | 0.92 | 2.64E−03 | 6.06E−03 |
| RPN1 | 0.92 | 8.01E−04 | 2.39E−03 |
| C1orf159 | 0.92 | 3.22E−03 | 7.11E−03 |
| RPL6 | 0.92 | 6.47E−05 | 4.03E−04 |
| CIAO1 | 0.92 | 1.63E−03 | 4.15E−03 |
| PSMA7 | 0.92 | 2.69E−04 | 1.07E−03 |
| CUL4A | 0.92 | 2.65E−03 | 6.07E−03 |
| MAGEA3 | 0.92 | 3.14E−03 | 6.97E−03 |
| BAD | 0.92 | 4.53E−03 | 9.27E−03 |
| NCK2 | 0.92 | 3.41E−03 | 7.43E−03 |
| NHP2 | 0.92 | 2.15E−04 | 9.04E−04 |
| YTHDF2 | 0.92 | 2.26E−03 | 5.36E−03 |
| ATN1 | 0.92 | 6.51E−04 | 2.06E−03 |
| ESYT1 | 0.92 | 4.56E−05 | 3.17E−04 |
| AKAP1 | 0.92 | 9.19E−04 | 2.65E−03 |
| CCNY | 0.92 | 2.58E−03 | 5.94E−03 |
| TRAFD1 | 0.91 | 9.85E−04 | 2.81E−03 |
| CIZ1 | 0.91 | 1.37E−03 | 3.61E−03 |
| RPL15 | 0.91 | 2.18E−04 | 9.17E−04 |
| NOLC1 | 0.91 | 4.59E−03 | 9.36E−03 |
| PRELID1 | 0.91 | 3.16E−04 | 1.20E−03 |
| CCT7 | 0.91 | 9.63E−05 | 5.29E−04 |
| ATF4 | 0.91 | 4.85E−03 | 9.76E−03 |
| RPL23A | 0.91 | 1.08E−04 | 5.69E−04 |
| MRPL10 | 0.91 | 1.01E−04 | 5.49E−04 |
| DAP3 | 0.91 | 2.02E−03 | 4.90E−03 |
| SMG5 | 0.91 | 4.86E−03 | 9.78E−03 |
| GCN1 | 0.91 | 5.85E−05 | 3.76E−04 |
| PDCD11 | 0.91 | 4.41E−05 | 3.10E−04 |
| LAMP1 | 0.91 | 7.60E−05 | 4.48E−04 |
| CPT2 | 0.91 | 3.38E−03 | 7.38E−03 |
| MRPS7 | 0.91 | 2.95E−03 | 6.61E−03 |
| DYRK1B | 0.91 | 3.96E−03 | 8.36E−03 |
| NCOA4 | 0.91 | 2.48E−03 | 5.76E−03 |
| NIPA2 | 0.91 | 4.61E−03 | 9.38E−03 |
| CHMP7 | 0.91 | 8.01E−04 | 2.39E−03 |
| ZNF638 | 0.91 | 1.66E−03 | 4.20E−03 |
| PITPNM1 | 0.91 | 6.21E−04 | 1.99E−03 |
| CIAPIN1 | 0.91 | 1.31E−03 | 3.50E−03 |
| NELFB | 0.91 | 1.44E−03 | 3.75E−03 |
| TUFM | 0.91 | 1.08E−03 | 3.02E−03 |
| WDR83OS | 0.91 | 1.21E−03 | 3.29E−03 |
| HCFC1 | 0.91 | 3.37E−03 | 7.36E−03 |
| USP14 | 0.91 | 1.60E−03 | 4.10E−03 |
| CASC3 | 0.91 | 2.98E−04 | 1.15E−03 |
| SDHC | 0.91 | 2.06E−03 | 4.98E−03 |
| RCC2 | 0.91 | 4.21E−03 | 8.77E−03 |
| FEM1B | 0.90 | 7.01E−04 | 2.18E−03 |
| NCAPH2 | 0.90 | 3.89E−03 | 8.24E−03 |
| CCAR2 | 0.90 | 1.96E−03 | 4.79E−03 |
| LAMTOR1 | 0.90 | 1.76E−03 | 4.39E−03 |
| CYCS | 0.90 | 2.18E−03 | 5.20E−03 |
| DNM2 | 0.90 | 3.99E−03 | 8.42E−03 |
| AXL | 0.90 | 1.71E−03 | 4.30E−03 |
| USP22 | 0.90 | 1.34E−03 | 3.56E−03 |
| SH3GL1 | 0.90 | 8.86E−04 | 2.58E−03 |
| SYNCRIP | 0.90 | 1.08E−03 | 3.01E−03 |
| BSG | 0.90 | 1.89E−03 | 4.65E−03 |
| RBM4 | 0.90 | 5.52E−04 | 1.83E−03 |
| CEP250 | 0.90 | 3.34E−06 | 6.66E−05 |
| KIAA0141 | 0.90 | 4.45E−03 | 9.14E−03 |
| ALDOA | 0.90 | 9.24E−05 | 5.14E−04 |
| SBNO1 | 0.90 | 4.83E−03 | 9.73E−03 |
| RABL6 | 0.90 | 1.38E−05 | 1.55E−04 |
| PLEKHO2 | 0.90 | 4.74E−03 | 9.58E−03 |
| HEXB | 0.90 | 4.21E−03 | 8.76E−03 |
| EID1 | 0.90 | 1.69E−03 | 4.26E−03 |
| NDUFA11 | 0.90 | 2.49E−03 | 5.77E−03 |
| NUP214 | 0.90 | 1.88E−03 | 4.62E−03 |
| UBAP2L | 0.90 | 2.54E−03 | 5.87E−03 |
| ABCD1 | 0.90 | 3.73E−03 | 7.96E−03 |
| ATAD3A | 0.90 | 4.75E−05 | 3.26E−04 |
| SNRPN | 0.90 | 1.45E−03 | 3.78E−03 |
| HUWE1 | 0.90 | 5.13E−05 | 3.42E−04 |
| BZW1 | 0.90 | 3.34E−03 | 7.31E−03 |
| ATP1A1 | 0.90 | 1.16E−04 | 5.91E−04 |
| PTGES2 | 0.90 | 1.32E−03 | 3.53E−03 |
| TMEM261 | 0.90 | 2.07E−03 | 4.99E−03 |
| PC | 0.90 | 1.88E−03 | 4.63E−03 |
| FUBP3 | 0.90 | 2.28E−03 | 5.40E−03 |
| RPL24 | 0.90 | 2.07E−04 | 8.85E−04 |
| ZMYND8 | 0.90 | 2.06E−03 | 4.98E−03 |
| NACA | 0.90 | 2.54E−04 | 1.03E−03 |
| CPSF1 | 0.90 | 2.92E−03 | 6.56E−03 |
| TARS2 | 0.90 | 4.18E−03 | 8.72E−03 |
| PARK7 | 0.90 | 4.29E−03 | 8.89E−03 |
| H1F0 | 0.90 | 2.88E−03 | 6.49E−03 |
| PQBP1 | 0.90 | 3.25E−03 | 7.16E−03 |
| SF3B2 | 0.90 | 2.20E−03 | 5.24E−03 |
| KDM3B | 0.90 | 3.69E−04 | 1.35E−03 |
| STAG2 | 0.90 | 1.68E−03 | 4.24E−03 |
| EIF3I | 0.90 | 5.59E−07 | 2.68E−05 |
| SNRNP200 | 0.90 | 2.66E−04 | 1.06E−03 |
| CDK2AP1 | 0.90 | 4.29E−03 | 8.90E−03 |
| PPM1G | 0.90 | 5.42E−04 | 1.80E−03 |
| GHITM | 0.90 | 3.68E−04 | 1.35E−03 |
| KMT2C | 0.90 | 4.12E−03 | 8.62E−03 |
| ARIH2 | 0.90 | 5.53E−05 | 3.61E−04 |
| VKORC1 | 0.90 | 4.66E−03 | 9.46E−03 |
| KPNA3 | 0.89 | 3.29E−05 | 2.59E−04 |
| ANO8 | 0.89 | 3.43E−03 | 7.45E−03 |
| TDG | 0.89 | 1.03E−03 | 2.89E−03 |
| BUB3 | 0.89 | 1.79E−03 | 4.46E−03 |
| MRPS2 | 0.89 | 2.75E−03 | 6.26E−03 |
| PAIP2 | 0.89 | 2.46E−03 | 5.74E−03 |
| SLC39A1 | 0.89 | 4.66E−04 | 1.62E−03 |
| OSBPL9 | 0.89 | 2.71E−03 | 6.17E−03 |
| SH3KBP1 | 0.89 | 2.98E−03 | 6.66E−03 |
| C20orf24 | 0.89 | 1.10E−03 | 3.06E−03 |
| PPP6R3 | 0.89 | 1.95E−04 | 8.49E−04 |
| ACSL3 | 0.89 | 2.12E−04 | 8.98E−04 |
| ADD1 | 0.89 | 2.47E−03 | 5.75E−03 |
| ZMIZ2 | 0.89 | 4.50E−03 | 9.22E−03 |
| HEG1 | 0.89 | 4.99E−03 | 1.00E−02 |
| PWP1 | 0.89 | 1.04E−03 | 2.92E−03 |
| PPP4C | 0.89 | 2.12E−04 | 8.98E−04 |
| LRRFIP2 | 0.89 | 3.05E−03 | 6.78E−03 |
| OPA1 | 0.89 | 3.92E−03 | 8.30E−03 |
| MEN1 | 0.89 | 1.26E−03 | 3.39E−03 |
| RSAD1 | 0.89 | 3.49E−03 | 7.56E−03 |
| MFN2 | 0.89 | 2.88E−04 | 1.12E−03 |
| TFE3 | 0.89 | 3.48E−05 | 2.68E−04 |
| DNAJA2 | 0.89 | 7.06E−04 | 2.19E−03 |
| HNRNPUL2 | 0.89 | 1.74E−03 | 4.35E−03 |
| EZR | 0.89 | 3.54E−03 | 7.63E−03 |
| CDK8 | 0.89 | 1.25E−03 | 3.38E−03 |
| LIPA | 0.89 | 3.75E−03 | 7.99E−03 |
| VARS | 0.89 | 3.96E−03 | 8.36E−03 |
| ACOX1 | 0.89 | 4.41E−03 | 9.09E−03 |
| RNPS1 | 0.89 | 6.85E−04 | 2.14E−03 |
| CHEK1 | 0.89 | 7.77E−04 | 2.33E−03 |
| VGF | 0.89 | 1.11E−03 | 3.07E−03 |
| MAGEA6 | 0.89 | 3.43E−03 | 7.45E−03 |
| MRPL3 | 0.89 | 1.82E−03 | 4.50E−03 |
| EIF2S1 | 0.89 | 9.90E−04 | 2.82E−03 |
| TOMM5 | 0.89 | 2.67E−03 | 6.11E−03 |
| LOC101927556 | 0.89 | 6.80E−04 | 2.13E−03 |
| SARIA | 0.89 | 7.51E−04 | 2.28E−03 |
| CISD3 | 0.89 | 8.35E−04 | 2.46E−03 |
| DARS | 0.89 | 2.72E−04 | 1.07E−03 |
| SRA1 | 0.89 | 2.33E−03 | 5.47E−03 |
| AZI2 | 0.89 | 1.92E−03 | 4.70E−03 |
| GPS1 | 0.89 | 3.53E−03 | 7.63E−03 |
| TMEM214 | 0.89 | 3.27E−03 | 7.19E−03 |
| CARHSP1 | 0.89 | 1.34E−03 | 3.57E−03 |
| HSP90AB1 | 0.89 | 7.43E−06 | 1.08E−04 |
| CSNK1G2 | 0.89 | 1.93E−03 | 4.73E−03 |
| LRWD1 | 0.89 | 4.81E−03 | 9.70E−03 |
| PHB2 | 0.89 | 4.46E−03 | 9.15E−03 |
| GSS | 0.89 | 1.41E−03 | 3.69E−03 |
| PPP1R37 | 0.89 | 8.65E−04 | 2.53E−03 |
| SUMO2 | 0.89 | 1.21E−03 | 3.28E−03 |
| STK3 | 0.89 | 4.38E−03 | 9.04E−03 |
| CPSF7 | 0.89 | 2.97E−03 | 6.65E−03 |
| CHP1 | 0.88 | 2.40E−05 | 2.10E−04 |
| SFRP1 | 0.88 | 6.41E−04 | 2.04E−03 |
| PSMD4 | 0.88 | 9.87E−04 | 2.81E−03 |
| TOP1MT | 0.88 | 2.18E−03 | 5.21E−03 |
| CHCHD3 | 0.88 | 7.46E−04 | 2.27E−03 |
| NADK | 0.88 | 7.45E−04 | 2.26E−03 |
| SPOP | 0.88 | 2.28E−04 | 9.46E−04 |
| AAGAB | 0.88 | 3.14E−03 | 6.97E−03 |
| CLTC | 0.88 | 2.24E−05 | 2.02E−04 |
| DNAJA1 | 0.88 | 1.91E−03 | 4.68E−03 |
| RPL36AL | 0.88 | 2.77E−03 | 6.29E−03 |
| CAPRIN1 | 0.88 | 4.62E−05 | 3.21E−04 |
| FUZ | 0.88 | 4.65E−03 | 9.45E−03 |
| DNAJB6 | 0.88 | 2.34E−03 | 5.51E−03 |
| DR1 | 0.88 | 3.44E−04 | 1.28E−03 |
| ATP5B | 0.88 | 1.91E−05 | 1.82E−04 |
| CREB3L2 | 0.88 | 2.22E−03 | 5.27E−03 |
| APP | 0.88 | 2.11E−05 | 1.94E−04 |
| HDAC3 | 0.88 | 1.60E−03 | 4.10E−03 |
| IQCE | 0.88 | 3.85E−04 | 1.39E−03 |
| SNTB2 | 0.88 | 2.88E−03 | 6.49E−03 |
| EML3 | 0.88 | 3.01E−04 | 1.15E−03 |
| SMARCC2 | 0.88 | 9.86E−04 | 2.81E−03 |
| UXT | 0.88 | 8.64E−04 | 2.52E−03 |
| RAD23B | 0.88 | 2.22E−03 | 5.28E−03 |
| MED16 | 0.88 | 4.38E−03 | 9.04E−03 |
| MCM3AP | 0.88 | 3.13E−05 | 2.52E−04 |
| EIF4G1 | 0.88 | 6.37E−04 | 2.03E−03 |
| PCBP1 | 0.88 | 3.20E−05 | 2.55E−04 |
| RBM28 | 0.88 | 3.79E−03 | 8.07E−03 |
| AGAP3 | 0.88 | 3.68E−03 | 7.87E−03 |
| EGLN1 | 0.88 | 2.01E−03 | 4.88E−03 |
| PSMD11 | 0.88 | 2.06E−03 | 4.98E−03 |
| PIGS | 0.88 | 8.30E−04 | 2.45E−03 |
| CDC42BPA | 0.88 | 2.84E−03 | 6.41E−03 |
| UTP6 | 0.88 | 1.96E−04 | 8.51E−04 |
| POLR2B | 0.88 | 2.08E−04 | 8.89E−04 |
| MCTS1 | 0.88 | 4.91E−03 | 9.86E−03 |
| WDR61 | 0.88 | 2.30E−03 | 5.42E−03 |
| SRP72 | 0.88 | 1.93E−03 | 4.73E−03 |
| ADNP2 | 0.88 | 4.63E−03 | 9.42E−03 |
| NRDC | 0.88 | 7.00E−04 | 2.17E−03 |
| DPYSL2 | 0.88 | 2.38E−03 | 5.57E−03 |
| NUBP1 | 0.88 | 8.38E−04 | 2.47E−03 |
| HNRNPUL1 | 0.88 | 4.64E−05 | 3.22E−04 |
| SLIRP | 0.88 | 3.16E−03 | 7.01E−03 |
| USP16 | 0.87 | 2.71E−03 | 6.17E−03 |
| GTF3C2 | 0.87 | 4.92E−03 | 9.88E−03 |
| SON | 0.87 | 1.65E−03 | 4.18E−03 |
| FAM136A | 0.87 | 4.01E−03 | 8.43E−03 |
| RAB8A | 0.87 | 4.28E−03 | 8.87E−03 |
| CCSAP | 0.87 | 2.95E−03 | 6.61E−03 |
| SRSF9 | 0.87 | 7.65E−04 | 2.31E−03 |
| UBE2G2 | 0.87 | 8.03E−04 | 2.39E−03 |
| HMG20A | 0.87 | 3.32E−03 | 7.28E−03 |
| SCAMP4 | 0.87 | 1.44E−04 | 6.88E−04 |
| TFCP2 | 0.87 | 2.14E−03 | 5.13E−03 |
| COPB2 | 0.87 | 8.94E−04 | 2.60E−03 |
| MTCH2 | 0.87 | 3.27E−03 | 7.18E−03 |
| CNBP | 0.87 | 1.57E−04 | 7.36E−04 |
| HIRA | 0.87 | 1.16E−03 | 3.18E−03 |
| PLPP2 | 0.87 | 2.29E−04 | 9.48E−04 |
| SPAG7 | 0.87 | 4.98E−03 | 9.98E−03 |
| SRM | 0.87 | 4.18E−04 | 1.48E−03 |
| OAZ2 | 0.87 | 1.35E−03 | 3.59E−03 |
| CLIC1 | 0.87 | 7.25E−04 | 2.22E−03 |
| U2AF2 | 0.87 | 8.75E−05 | 4.95E−04 |
| CYC1 | 0.87 | 2.11E−04 | 8.95E−04 |
| ENSA | 0.87 | 4.51E−03 | 9.23E−03 |
| IGF2BP2 | 0.87 | 3.41E−03 | 7.43E−03 |
| KARS | 0.87 | 1.36E−04 | 6.67E−04 |
| MRPL51 | 0.87 | 2.88E−03 | 6.49E−03 |
| PSENEN | 0.87 | 3.03E−03 | 6.75E−03 |
| TRAK1 | 0.87 | 2.90E−03 | 6.53E−03 |
| ZNF207 | 0.87 | 1.96E−05 | 1.86E−04 |
| PKM | 0.87 | 1.51E−05 | 1.61E−04 |
| CNPY2 | 0.87 | 3.24E−04 | 1.22E−03 |
| BBS4 | 0.87 | 2.02E−03 | 4.90E−03 |
| USP10 | 0.87 | 3.39E−03 | 7.38E−03 |
| TRIP10 | 0.87 | 6.96E−04 | 2.17E−03 |
| FBXO21 | 0.87 | 5.24E−04 | 1.75E−03 |
| PLAA | 0.87 | 2.90E−03 | 6.54E−03 |
| VPRBP | 0.87 | 1.47E−03 | 3.82E−03 |
| DDX20 | 0.87 | 4.02E−03 | 8.45E−03 |
| FAM120A | 0.87 | 2.00E−05 | 1.87E−04 |
| SAMM50 | 0.87 | 1.52E−03 | 3.93E−03 |
| SENP3 | 0.87 | 6.50E−04 | 2.06E−03 |
| ADCY6 | 0.87 | 4.51E−03 | 9.24E−03 |
| GDE1 | 0.87 | 8.67E−04 | 2.53E−03 |
| PSMD13 | 0.87 | 1.98E−04 | 8.57E−04 |
| CSNK2A1 | 0.87 | 5.30E−04 | 1.77E−03 |
| EIF2AK4 | 0.87 | 8.38E−04 | 2.47E−03 |
| PHF20 | 0.87 | 3.99E−05 | 2.91E−04 |
| ATP2B1 | 0.87 | 3.55E−03 | 7.64E−03 |
| C11orf31 | 0.87 | 8.26E−05 | 4.74E−04 |
| TUT1 | 0.87 | 2.39E−03 | 5.59E−03 |
| NOC2L | 0.87 | 7.37E−05 | 4.39E−04 |
| C14orf166 | 0.87 | 1.55E−03 | 3.99E−03 |
| HMGA1 | 0.87 | 2.59E−05 | 2.21E−04 |
| CUL1 | 0.87 | 5.87E−04 | 1.91E−03 |
| UPF3A | 0.87 | 2.69E−03 | 6.15E−03 |
| GSPT1 | 0.87 | 2.37E−03 | 5.56E−03 |
| PSMB1 | 0.87 | 4.96E−06 | 8.44E−05 |
| AHCYL1 | 0.87 | 3.83E−04 | 1.39E−03 |
| ESYT2 | 0.87 | 2.86E−05 | 2.38E−04 |
| VDAC1 | 0.87 | 2.99E−04 | 1.15E−03 |
| GNG12 | 0.87 | 5.71E−07 | 2.69E−05 |
| RPL5 | 0.87 | 3.87E−05 | 2.86E−04 |
| RARS | 0.87 | 1.95E−03 | 4.76E−03 |
| PPM1B | 0.87 | 3.00E−04 | 1.15E−03 |
| DDX3X | 0.87 | 6.85E−04 | 2.14E−03 |
| SUMO1 | 0.87 | 2.29E−03 | 5.40E−03 |
| TNPO3 | 0.87 | 1.40E−03 | 3.68E−03 |
| PIK3R1 | 0.87 | 7.13E−04 | 2.20E−03 |
| C5orf15 | 0.87 | 1.56E−03 | 4.01E−03 |
| ARFGAP2 | 0.87 | 3.35E−04 | 1.25E−03 |
| G6PD | 0.87 | 4.43E−05 | 3.10E−04 |
| PAM | 0.87 | 1.49E−03 | 3.87E−03 |
| SERPINB6 | 0.87 | 3.24E−03 | 7.14E−03 |
| RNF40 | 0.86 | 2.86E−05 | 2.38E−04 |
| MRPS34 | 0.86 | 4.45E−03 | 9.14E−03 |
| SLC4A2 | 0.86 | 7.92E−04 | 2.37E−03 |
| ARL2 | 0.86 | 2.73E−03 | 6.22E−03 |
| ELP2 | 0.86 | 2.72E−03 | 6.21E−03 |
| YY1 | 0.86 | 9.61E−04 | 2.75E−03 |
| HNRNPA1 | 0.86 | 9.70E−05 | 5.32E−04 |
| WDR43 | 0.86 | 1.00E−03 | 2.85E−03 |
| DUT | 0.86 | 1.38E−03 | 3.63E−03 |
| RPS26 | 0.86 | 1.86E−04 | 8.24E−04 |
| ZNF395 | 0.86 | 4.16E−03 | 8.68E−03 |
| MSTO1 | 0.86 | 4.31E−03 | 8.91E−03 |
| PPHLN1 | 0.86 | 2.47E−03 | 5.74E−03 |
| KIAA1429 | 0.86 | 1.19E−03 | 3.24E−03 |
| CREBBP | 0.86 | 2.23E−03 | 5.29E−03 |
| EIF1B | 0.86 | 2.31E−03 | 5.45E−03 |
| CHRAC1 | 0.86 | 1.16E−03 | 3.18E−03 |
| PHLDA3 | 0.86 | 2.51E−04 | 1.02E−03 |
| REPIN1 | 0.86 | 1.56E−03 | 4.02E−03 |
| SHFM1 | 0.86 | 3.49E−03 | 7.57E−03 |
| COX20 | 0.86 | 4.95E−03 | 9.92E−03 |
| POLR2K | 0.86 | 2.38E−03 | 5.57E−03 |
| NFATC3 | 0.86 | 4.24E−04 | 1.50E−03 |
| MGA | 0.86 | 2.98E−03 | 6.66E−03 |
| ENTPD6 | 0.86 | 2.01E−04 | 8.64E−04 |
| RNASEH2C | 0.86 | 2.80E−03 | 6.34E−03 |
| C17orf80 | 0.86 | 2.12E−03 | 5.09E−03 |
| TRA2A | 0.86 | 2.79E−03 | 6.32E−03 |
| UBE2N | 0.86 | 1.28E−04 | 6.37E−04 |
| TNRC18 | 0.86 | 2.48E−03 | 5.76E−03 |
| EI24 | 0.86 | 8.07E−04 | 2.40E−03 |
| ABCD3 | 0.86 | 1.60E−03 | 4.09E−03 |
| IARS | 0.86 | 1.64E−05 | 1.68E−04 |
| UBE20 | 0.86 | 1.40E−03 | 3.67E−03 |
| SUPT7L | 0.86 | 1.73E−03 | 4.34E−03 |
| GOT2 | 0.86 | 1.16E−05 | 1.40E−04 |
| PSMA6 | 0.86 | 1.01E−03 | 2.87E−03 |
| COPS2 | 0.86 | 1.81E−03 | 4.48E−03 |
| PSMC1 | 0.86 | 2.20E−03 | 5.23E−03 |
| DDX50 | 0.86 | 2.08E−03 | 5.00E−03 |
| SLBP | 0.86 | 2.04E−03 | 4.93E−03 |
| TEAD1 | 0.86 | 3.30E−03 | 7.24E−03 |
| GPBP1 | 0.86 | 1.04E−04 | 5.59E−04 |
| CNP | 0.86 | 1.41E−05 | 1.57E−04 |
| CLUH | 0.86 | 5.99E−04 | 1.93E−03 |
| APIS | 0.86 | 2.24E−04 | 9.34E−04 |
| UBE2M | 0.86 | 7.96E−04 | 2.38E−03 |
| CCDC47 | 0.86 | 2.22E−03 | 5.28E−03 |
| FXR1 | 0.86 | 3.80E−03 | 8.08E−03 |
| RPUSD2 | 0.86 | 2.53E−03 | 5.86E−03 |
| CAND1 | 0.86 | 5.56E−04 | 1.83E−03 |
| SHMT2 | 0.86 | 2.54E−04 | 1.03E−03 |
| COLGALT1 | 0.86 | 6.62E−05 | 4.08E−04 |
| DHX40 | 0.86 | 7.68E−04 | 2.32E−03 |
| TRMT10C | 0.86 | 8.70E−04 | 2.54E−03 |
| PHF14 | 0.86 | 2.40E−03 | 5.62E−03 |
| ZNF106 | 0.86 | 3.35E−04 | 1.25E−03 |
| CS | 0.86 | 2.64E−04 | 1.05E−03 |
| PSMD7 | 0.86 | 4.55E−04 | 1.58E−03 |
| URM1 | 0.86 | 3.26E−03 | 7.17E−03 |
| LSM12 | 0.86 | 1.82E−05 | 1.77E−04 |
| TRUB2 | 0.86 | 2.95E−03 | 6.61E−03 |
| LONP1 | 0.86 | 1.87E−04 | 8.26E−04 |
| MED1 | 0.86 | 8.25E−04 | 2.44E−03 |
| PLCD3 | 0.86 | 1.63E−03 | 4.15E−03 |
| TRIM28 | 0.86 | 4.44E−05 | 3.11E−04 |
| ATP5G1 | 0.86 | 4.91E−03 | 9.86E−03 |
| ATP50 | 0.86 | 2.69E−04 | 1.07E−03 |
| SDHA | 0.86 | 2.87E−04 | 1.12E−03 |
| NF1 | 0.86 | 3.15E−03 | 6.98E−03 |
| UBE2K | 0.86 | 1.76E−04 | 7.91E−04 |
| PRKACA | 0.86 | 9.75E−04 | 2.79E−03 |
| NDUFB9 | 0.86 | 3.45E−05 | 2.67E−04 |
| YME1L1 | 0.86 | 6.81E−04 | 2.13E−03 |
| CPNE1 | 0.86 | 4.33E−03 | 8.94E−03 |
| NELFA | 0.86 | 2.06E−04 | 8.82E−04 |
| MICU1 | 0.86 | 1.04E−03 | 2.92E−03 |
| GPI | 0.85 | 1.21E−04 | 6.12E−04 |
| TOP1 | 0.85 | 1.44E−05 | 1.57E−04 |
| ARMC8 | 0.85 | 3.62E−03 | 7.76E−03 |
| GEMIN4 | 0.85 | 5.10E−04 | 1.72E−03 |
| COPG2 | 0.85 | 3.52E−03 | 7.61E−03 |
| ATP5G2 | 0.85 | 2.45E−04 | 1.00E−03 |
| YBX1 | 0.85 | 3.78E−06 | 7.17E−05 |
| DDX54 | 0.85 | 2.08E−04 | 8.88E−04 |
| RRN3 | 0.85 | 2.08E−04 | 8.89E−04 |
| WAC | 0.85 | 6.15E−04 | 1.98E−03 |
| RYK | 0.85 | 8.09E−05 | 4.67E−04 |
| CPSF6 | 0.85 | 5.05E−05 | 3.38E−04 |
| PSMD1 | 0.85 | 4.40E−03 | 9.06E−03 |
| TPRKB | 0.85 | 5.35E−04 | 1.78E−03 |
| PDIA4 | 0.85 | 8.41E−05 | 4.81E−04 |
| CT45A1 | 0.85 | 4.93E−03 | 9.89E−03 |
| TBRG4 | 0.85 | 5.74E−05 | 3.71E−04 |
| TCAIM | 0.85 | 2.64E−03 | 6.06E−03 |
| BYSL | 0.85 | 3.76E−04 | 1.37E−03 |
| 7-Sep | 0.85 | 1.82E−03 | 4.50E−03 |
| PSMA5 | 0.85 | 1.55E−04 | 7.29E−04 |
| PCCB | 0.85 | 2.52E−03 | 5.84E−03 |
| INO80 | 0.85 | 1.29E−03 | 3.46E−03 |
| CSTF2T | 0.85 | 4.72E−03 | 9.56E−03 |
| TOMM70 | 0.85 | 1.09E−03 | 3.04E−03 |
| PHC1 | 0.85 | 1.00E−03 | 2.84E−03 |
| IPO8 | 0.85 | 8.48E−04 | 2.49E−03 |
| MDH2 | 0.85 | 3.91E−05 | 2.88E−04 |
| ZNF664 | 0.85 | 2.88E−04 | 1.12E−03 |
| DHX29 | 0.85 | 1.93E−03 | 4.73E−03 |
| TCTA | 0.85 | 2.88E−03 | 6.50E−03 |
| SEC11A | 0.85 | 7.46E−05 | 4.43E−04 |
| OBSL1 | 0.85 | 2.17E−03 | 5.18E−03 |
| TPGS2 | 0.85 | 7.16E−04 | 2.21E−03 |
| NUP153 | 0.85 | 2.25E−03 | 5.34E−03 |
| SRRM1 | 0.85 | 1.36E−03 | 3.61E−03 |
| ASF1A | 0.85 | 2.55E−03 | 5.89E−03 |
| DNAJA3 | 0.85 | 7.14E−04 | 2.20E−03 |
| DOCK1 | 0.85 | 1.31E−03 | 3.50E−03 |
| GTF2A2 | 0.85 | 1.32E−03 | 3.53E−03 |
| WBSCR22 | 0.85 | 2.32E−03 | 5.47E−03 |
| HNRNPK | 0.85 | 9.85E−07 | 3.66E−05 |
| SOX5 | 0.85 | 8.86E−04 | 2.58E−03 |
| MRPS24 | 0.85 | 1.17E−03 | 3.21E−03 |
| HNRNPAO | 0.85 | 1.98E−04 | 8.58E−04 |
| URB2 | 0.85 | 6.91E−04 | 2.16E−03 |
| IGF2BP1 | 0.85 | 3.87E−05 | 2.86E−04 |
| COMMD4 | 0.85 | 2.87E−05 | 2.38E−04 |
| NOL6 | 0.85 | 1.42E−04 | 6.83E−04 |
| RBBP7 | 0.85 | 6.59E−04 | 2.08E−03 |
| UCHLS | 0.85 | 2.82E−03 | 6.39E−03 |
| C12orf10 | 0.85 | 8.89E−04 | 2.58E−03 |
| PRKAR2A | 0.85 | 3.08E−04 | 1.18E−03 |
| ADI1 | 0.85 | 2.47E−03 | 5.74E−03 |
| ENO1 | 0.85 | 2.63E−06 | 5.95E−05 |
| PSMA1 | 0.85 | 1.08E−03 | 3.01E−03 |
| COQ5 | 0.85 | 1.83E−03 | 4.51E−03 |
| CBX5 | 0.85 | 1.37E−04 | 6.69E−04 |
| SF1 | 0.85 | 3.39E−03 | 7.40E−03 |
| GPHN | 0.85 | 1.49E−03 | 3.86E−03 |
| USP7 | 0.85 | 2.26E−04 | 9.40E−04 |
| PELP1 | 0.85 | 2.38E−04 | 9.77E−04 |
| SEC23IP | 0.85 | 3.45E−04 | 1.28E−03 |
| VKORC1L1 | 0.85 | 5.50E−04 | 1.82E−03 |
| KHDRBS1 | 0.85 | 2.97E−04 | 1.15E−03 |
| THOP1 | 0.85 | 6.84E−04 | 2.14E−03 |
| SLC30A5 | 0.85 | 1.06E−03 | 2.98E−03 |
| BTF3 | 0.85 | 5.47E−04 | 1.81E−03 |
| CSTF1 | 0.85 | 1.73E−03 | 4.34E−03 |
| GATC | 0.85 | 1.68E−03 | 4.25E−03 |
| AASDHPPT | 0.85 | 4.74E−03 | 9.59E−03 |
| AAR2 | 0.84 | 1.29E−04 | 6.38E−04 |
| EIF2B5 | 0.84 | 3.34E−03 | 7.31E−03 |
| SLC50A1 | 0.84 | 7.76E−05 | 4.55E−04 |
| MCCC2 | 0.84 | 1.30E−04 | 6.42E−04 |
| DHX30 | 0.84 | 5.62E−06 | 9.02E−05 |
| MFSD14A | 0.84 | 1.54E−03 | 3.97E−03 |
| UMPS | 0.84 | 3.30E−04 | 1.24E−03 |
| RBM10 | 0.84 | 2.00E−03 | 4.86E−03 |
| FAM118B | 0.84 | 3.35E−03 | 7.32E−03 |
| NOC4L | 0.84 | 7.95E−04 | 2.38E−03 |
| TRMT5 | 0.84 | 2.65E−03 | 6.08E−03 |
| ADAM9 | 0.84 | 1.37E−03 | 3.62E−03 |
| PAGR1 | 0.84 | 2.13E−04 | 9.00E−04 |
| FAM63B | 0.84 | 2.46E−03 | 5.74E−03 |
| CCT8 | 0.84 | 1.20E−03 | 3.25E−03 |
| NDUFB10 | 0.84 | 2.39E−03 | 5.58E−03 |
| NDUFAB1 | 0.84 | 7.96E−04 | 2.38E−03 |
| NUTF2 | 0.84 | 5.61E−04 | 1.84E−03 |
| SNRPD2 | 0.84 | 4.02E−05 | 2.92E−04 |
| NOB1 | 0.84 | 4.17E−04 | 1.48E−03 |
| AKT2 | 0.84 | 1.42E−03 | 3.73E−03 |
| MRPL34 | 0.84 | 4.87E−04 | 1.67E−03 |
| USP6NL | 0.84 | 5.95E−04 | 1.93E−03 |
| ICE1 | 0.84 | 1.44E−03 | 3.75E−03 |
| MAZ | 0.84 | 1.16E−03 | 3.19E−03 |
| LAMP2 | 0.84 | 1.21E−03 | 3.28E−03 |
| PTBP1 | 0.84 | 5.08E−05 | 3.40E−04 |
| THOC5 | 0.84 | 1.15E−03 | 3.15E−03 |
| KIF3B | 0.84 | 1.41E−04 | 6.81E−04 |
| POM121C | 0.84 | 1.04E−05 | 1.30E−04 |
| GULP1 | 0.84 | 1.81E−03 | 4.48E−03 |
| AHCTF1 | 0.84 | 9.30E−04 | 2.68E−03 |
| HNRNPL | 0.84 | 2.51E−05 | 2.16E−04 |
| SERBP1 | 0.84 | 3.46E−05 | 2.68E−04 |
| SRSF10 | 0.84 | 4.15E−03 | 8.67E−03 |
| POM121 | 0.84 | 7.93E−04 | 2.37E−03 |
| ATPSE | 0.84 | 2.67E−04 | 1.06E−03 |
| ETF1 | 0.84 | 2.39E−05 | 2.10E−04 |
| ARNT2 | 0.84 | 1.68E−03 | 4.24E−03 |
| DHX15 | 0.84 | 1.95E−03 | 4.76E−03 |
| MRPL15 | 0.84 | 2.16E−03 | 5.16E−03 |
| ZMIZ1 | 0.84 | 1.96E−04 | 8.52E−04 |
| NSUN2 | 0.84 | 4.44E−03 | 9.13E−03 |
| STOML2 | 0.84 | 4.09E−04 | 1.46E−03 |
| CXorf56 | 0.84 | 1.45E−03 | 3.79E−03 |
| MDN1 | 0.84 | 1.20E−04 | 6.08E−04 |
| PTPN2 | 0.84 | 1.87E−03 | 4.60E−03 |
| SF3B1 | 0.84 | 1.41E−04 | 6.80E−04 |
| CHTOP | 0.84 | 7.92E−04 | 2.37E−03 |
| MSANTD4 | 0.84 | 8.03E−04 | 2.39E−03 |
| DKC1 | 0.84 | 6.18E−04 | 1.98E−03 |
| BAZ1B | 0.84 | 4.84E−05 | 3.29E−04 |
| RBM25 | 0.84 | 1.86E−03 | 4.58E−03 |
| CCT2 | 0.84 | 9.67E−05 | 5.30E−04 |
| AKT1S1 | 0.84 | 2.26E−04 | 9.37E−04 |
| ELOF1 | 0.84 | 1.34E−04 | 6.58E−04 |
| MED26 | 0.84 | 8.43E−04 | 2.48E−03 |
| XPO7 | 0.84 | 1.68E−03 | 4.25E−03 |
| NFXL1 | 0.84 | 1.07E−03 | 2.99E−03 |
| COPZ1 | 0.84 | 4.33E−04 | 1.52E−03 |
| LOC100288778 | 0.84 | 2.23E−03 | 5.29E−03 |
| HNRNPAB | 0.84 | 5.39E−04 | 1.79E−03 |
| CUL3 | 0.84 | 6.93E−04 | 2.16E−03 |
| SNRPD3 | 0.84 | 2.50E−03 | 5.79E−03 |
| RAVER1 | 0.84 | 3.20E−05 | 2.55E−04 |
| PDPR | 0.84 | 1.58E−04 | 7.38E−04 |
| HSPA8 | 0.84 | 1.54E−06 | 4.67E−05 |
| MAP4K5 | 0.84 | 1.89E−03 | 4.65E−03 |
| EPS15L1 | 0.84 | 1.10E−03 | 3.04E−03 |
| CYB561 | 0.84 | 1.53E−03 | 3.96E−03 |
| DSTN | 0.84 | 4.97E−05 | 3.35E−04 |
| THUMPD1 | 0.84 | 3.03E−03 | 6.76E−03 |
| SLC39A14 | 0.84 | 2.31E−05 | 2.05E−04 |
| CHD4 | 0.84 | 4.71E−06 | 8.20E−05 |
| FANCG | 0.84 | 3.43E−03 | 7.45E−03 |
| CLK2 | 0.84 | 4.05E−03 | 8.49E−03 |
| TYW1 | 0.84 | 4.82E−03 | 9.72E−03 |
| PTOV1 | 0.84 | 3.53E−03 | 7.63E−03 |
| CDC123 | 0.84 | 3.43E−04 | 1.28E−03 |
| ING1 | 0.84 | 8.06E−04 | 2.40E−03 |
| SRRM2 | 0.84 | 7.82E−05 | 4.57E−04 |
| PRMT3 | 0.84 | 1.99E−03 | 4.85E−03 |
| LOC613037 | 0.84 | 3.31E−03 | 7.26E−03 |
| ACADVL | 0.84 | 6.41E−04 | 2.04E−03 |
| MSH6 | 0.84 | 7.52E−04 | 2.28E−03 |
| PIK3R4 | 0.84 | 4.80E−05 | 3.27E−04 |
| TOMM6 | 0.84 | 5.93E−04 | 1.92E−03 |
| PDHB | 0.84 | 1.70E−05 | 1.71E−04 |
| MRPS27 | 0.84 | 1.86E−03 | 4.58E−03 |
| ABHD12 | 0.84 | 1.04E−03 | 2.93E−03 |
| HIF1A | 0.84 | 1.23E−05 | 1.45E−04 |
| BCKDHA | 0.84 | 3.96E−03 | 8.36E−03 |
| CASP7 | 0.84 | 7.41E−04 | 2.26E−03 |
| SIKE1 | 0.84 | 4.28E−03 | 8.88E−03 |
| VAV2 | 0.84 | 2.14E−04 | 9.04E−04 |
| CLPB | 0.84 | 2.20E−03 | 5.24E−03 |
| AIMP1 | 0.84 | 3.71E−04 | 1.36E−03 |
| PON2 | 0.84 | 7.08E−04 | 2.19E−03 |
| NEMF | 0.84 | 4.74E−03 | 9.59E−03 |
| SRSF5 | 0.84 | 1.71E−03 | 4.30E−03 |
| TUBB4B | 0.84 | 1.87E−05 | 1.80E−04 |
| CARM1 | 0.84 | 9.56E−04 | 2.74E−03 |
| YY1AP1 | 0.83 | 2.64E−03 | 6.06E−03 |
| DDX27 | 0.83 | 4.76E−04 | 1.64E−03 |
| E2F1 | 0.83 | 4.79E−04 | 1.65E−03 |
| RFX5 | 0.83 | 2.31E−04 | 9.53E−04 |
| RPS6KA3 | 0.83 | 6.13E−05 | 3.88E−04 |
| ATP5G3 | 0.83 | 4.84E−04 | 1.66E−03 |
| CCNJL | 0.83 | 1.01E−03 | 2.87E−03 |
| UBN1 | 0.83 | 7.04E−05 | 4.26E−04 |
| GTF2F1 | 0.83 | 2.24E−04 | 9.35E−04 |
| PIP5K1A | 0.83 | 1.20E−03 | 3.26E−03 |
| NEK4 | 0.83 | 9.83E−04 | 2.81E−03 |
| B4GAT1 | 0.83 | 3.13E−03 | 6.94E−03 |
| TFDP2 | 0.83 | 2.54E−03 | 5.87E−03 |
| CCT3 | 0.83 | 2.11E−04 | 8.95E−04 |
| PI4KB | 0.83 | 1.69E−04 | 7.71E−04 |
| ERLIN1 | 0.83 | 8.16E−04 | 2.42E−03 |
| HMGN1 | 0.83 | 4.25E−04 | 1.50E−03 |
| SLC44A1 | 0.83 | 3.61E−03 | 7.76E−03 |
| GCLC | 0.83 | 3.33E−03 | 7.30E−03 |
| BABAM1 | 0.83 | 1.08E−04 | 5.68E−04 |
| CBX3 | 0.83 | 7.67E−04 | 2.32E−03 |
| NOP58 | 0.83 | 1.67E−04 | 7.65E−04 |
| BCS1L | 0.83 | 1.30E−04 | 6.42E−04 |
| SLC1A5 | 0.83 | 5.20E−04 | 1.74E−03 |
| DRG1 | 0.83 | 2.34E−03 | 5.49E−03 |
| ATG4B | 0.83 | 1.26E−03 | 3.39E−03 |
| TXNRD2 | 0.83 | 2.20E−03 | 5.23E−03 |
| KEAP1 | 0.83 | 2.04E−03 | 4.94E−03 |
| SLX1A | 0.83 | 1.68E−03 | 4.24E−03 |
| SLX1B | 0.83 | 1.68E−03 | 4.24E−03 |
| NOL10 | 0.83 | 3.62E−03 | 7.77E−03 |
| DERA | 0.83 | 1.42E−05 | 1.57E−04 |
| NIP7 | 0.83 | 7.17E−04 | 2.21E−03 |
| GMPR2 | 0.83 | 2.45E−03 | 5.71E−03 |
| SMPD4 | 0.83 | 9.55E−04 | 2.74E−03 |
| MAPK1IP1L | 0.83 | 9.16E−04 | 2.64E−03 |
| DHX33 | 0.83 | 7.81E−04 | 2.34E−03 |
| RAF1 | 0.83 | 5.36E−04 | 1.79E−03 |
| C16orf13 | 0.83 | 2.47E−05 | 2.14E−04 |
| RBM8A | 0.83 | 5.81E−05 | 3.74E−04 |
| USP34 | 0.83 | 6.57E−05 | 4.07E−04 |
| PEX26 | 0.83 | 2.36E−03 | 5.54E−03 |
| PTP4A2 | 0.83 | 1.08E−03 | 3.00E−03 |
| ENY2 | 0.83 | 2.84E−03 | 6.42E−03 |
| RAP1A | 0.83 | 4.20E−04 | 1.49E−03 |
| USP24 | 0.83 | 8.11E−04 | 2.41E−03 |
| GNG5 | 0.83 | 3.06E−03 | 6.81E−03 |
| CEBPZ | 0.83 | 3.15E−04 | 1.20E−03 |
| PSMC3 | 0.83 | 4.90E−05 | 3.32E−04 |
| SRP9 | 0.83 | 3.05E−04 | 1.17E−03 |
| PAPOLA | 0.83 | 8.08E−04 | 2.40E−03 |
| SPCS1 | 0.83 | 1.33E−04 | 6.54E−04 |
| IRAK1 | 0.83 | 6.02E−05 | 3.84E−04 |
| H2AFX | 0.83 | 3.82E−03 | 8.12E−03 |
| EWSR1 | 0.83 | 6.52E−05 | 4.05E−04 |
| NCOA3 | 0.83 | 2.08E−05 | 1.92E−04 |
| KIAA1143 | 0.83 | 1.89E−03 | 4.64E−03 |
| HSP90B1 | 0.83 | 1.90E−04 | 8.33E−04 |
| PSMD5 | 0.83 | 2.91E−04 | 1.13E−03 |
| DDX18 | 0.83 | 9.63E−04 | 2.76E−03 |
| RSL24D1 | 0.83 | 1.65E−03 | 4.18E−03 |
| NFIC | 0.83 | 3.16E−04 | 1.20E−03 |
| PSMB3 | 0.83 | 1.30E−04 | 6.42E−04 |
| TBK1 | 0.83 | 8.52E−04 | 2.50E−03 |
| TRIAP1 | 0.83 | 1.81E−03 | 4.48E−03 |
| SRGAP2B | 0.83 | 4.35E−03 | 8.98E−03 |
| TNKS1BP1 | 0.83 | 6.78E−05 | 4.17E−04 |
| PUSL1 | 0.83 | 4.48E−04 | 1.57E−03 |
| SDHB | 0.83 | 9.61E−04 | 2.75E−03 |
| RPAP3 | 0.83 | 1.17E−03 | 3.21E−03 |
| FDX1 | 0.83 | 5.33E−04 | 1.78E−03 |
| TIMP2 | 0.83 | 1.97E−04 | 8.53E−04 |
| ANKRD40 | 0.83 | 4.12E−03 | 8.62E−03 |
| CDC5L | 0.83 | 3.56E−03 | 7.67E−03 |
| CALR | 0.83 | 9.78E−06 | 1.27E−04 |
| PIM2 | 0.83 | 1.01E−03 | 2.87E−03 |
| MRM3 | 0.83 | 3.55E−03 | 7.64E−03 |
| CNOT1 | 0.83 | 2.63E−04 | 1.05E−03 |
| CDK4 | 0.83 | 2.13E−05 | 1.95E−04 |
| ORAI2 | 0.83 | 4.29E−04 | 1.51E−03 |
| POLDIP2 | 0.83 | 6.60E−05 | 4.07E−04 |
| MAPK8IP3 | 0.83 | 3.05E−03 | 6.79E−03 |
| UTP11 | 0.83 | 2.49E−04 | 1.01E−03 |
| FAM49B | 0.83 | 2.91E−03 | 6.54E−03 |
| CACUL1 | 0.83 | 2.64E−03 | 6.06E−03 |
| POLDIP3 | 0.83 | 1.00E−03 | 2.84E−03 |
| NBPF1 | 0.83 | 1.69E−03 | 4.26E−03 |
| C1orf123 | 0.82 | 1.67E−04 | 7.65E−04 |
| MTG2 | 0.82 | 7.21E−05 | 4.31E−04 |
| SEC13 | 0.82 | 3.22E−04 | 1.21E−03 |
| HDGF | 0.82 | 2.53E−04 | 1.02E−03 |
| ALKBH1 | 0.82 | 1.42E−03 | 3.71E−03 |
| BDP1 | 0.82 | 2.32E−03 | 5.46E−03 |
| MARCKSL1 | 0.82 | 1.81E−05 | 1.77E−04 |
| RBFOX2 | 0.82 | 6.52E−04 | 2.06E−03 |
| HEIH | 0.82 | 2.29E−04 | 9.47E−04 |
| ADGRL2 | 0.82 | 3.61E−05 | 2.72E−04 |
| SMARCC1 | 0.82 | 1.08E−05 | 1.34E−04 |
| CNTROB | 0.82 | 9.14E−05 | 5.10E−04 |
| CLTB | 0.82 | 4.30E−03 | 8.90E−03 |
| NFYC | 0.82 | 1.50E−03 | 3.88E−03 |
| C19orf53 | 0.82 | 5.42E−04 | 1.80E−03 |
| PHB | 0.82 | 2.43E−04 | 9.94E−04 |
| ERH | 0.82 | 1.57E−05 | 1.66E−04 |
| TIMM8B | 0.82 | 1.38E−03 | 3.64E−03 |
| EPRS | 0.82 | 2.28E−06 | 5.53E−05 |
| PNPLA6 | 0.82 | 8.36E−05 | 4.79E−04 |
| NSD1 | 0.82 | 4.94E−05 | 3.33E−04 |
| PRPF40A | 0.82 | 2.29E−05 | 2.05E−04 |
| AFG3L2 | 0.82 | 1.44E−03 | 3.77E−03 |
| DCLRE1A | 0.82 | 4.00E−03 | 8.42E−03 |
| SLC31A1 | 0.82 | 2.22E−04 | 9.26E−04 |
| ILVBL | 0.82 | 1.43E−05 | 1.57E−04 |
| POLR2J | 0.82 | 5.53E−04 | 1.83E−03 |
| MED13L | 0.82 | 2.69E−04 | 1.07E−03 |
| NUP155 | 0.82 | 2.54E−03 | 5.87E−03 |
| EMC4 | 0.82 | 2.42E−05 | 2.10E−04 |
| RIF1 | 0.82 | 2.29E−03 | 5.40E−03 |
| CCT4 | 0.82 | 1.41E−04 | 6.82E−04 |
| WDR77 | 0.82 | 2.73E−04 | 1.08E−03 |
| C5orf22 | 0.82 | 1.37E−03 | 3.61E−03 |
| ELF2 | 0.82 | 5.38E−04 | 1.79E−03 |
| FOXRED1 | 0.82 | 4.92E−05 | 3.32E−04 |
| TAF1C | 0.82 | 4.99E−04 | 1.70E−03 |
| ARPIN | 0.82 | 5.42E−04 | 1.80E−03 |
| POLRIB | 0.82 | 2.61E−04 | 1.05E−03 |
| CHMP2B | 0.82 | 3.64E−03 | 7.80E−03 |
| ORC5 | 0.82 | 1.23E−03 | 3.32E−03 |
| FIP1L1 | 0.82 | 1.77E−03 | 4.42E−03 |
| DNMBP | 0.82 | 3.83E−03 | 8.14E−03 |
| GMPS | 0.82 | 3.63E−04 | 1.34E−03 |
| CLIC4 | 0.82 | 1.38E−04 | 6.70E−04 |
| KIF22 | 0.82 | 2.64E−03 | 6.06E−03 |
| FANCI | 0.82 | 1.25E−03 | 3.38E−03 |
| EIF2B1 | 0.82 | 9.45E−07 | 3.58E−05 |
| MAPRE2 | 0.82 | 8.88E−04 | 2.58E−03 |
| UBR5 | 0.82 | 1.25E−03 | 3.37E−03 |
| SUMO3 | 0.82 | 7.96E−04 | 2.38E−03 |
| PPAT | 0.82 | 7.62E−04 | 2.30E−03 |
| GPX4 | 0.82 | 9.24E−04 | 2.66E−03 |
| TRNT1 | 0.82 | 1.56E−05 | 1.65E−04 |
| LDHA | 0.82 | 1.27E−05 | 1.48E−04 |
| HMGCS1 | 0.82 | 5.11E−04 | 1.72E−03 |
| FAF1 | 0.82 | 1.61E−03 | 4.11E−03 |
| AHR | 0.82 | 1.71E−04 | 7.76E−04 |
| ANXA11 | 0.82 | 2.95E−05 | 2.42E−04 |
| EPB41L1 | 0.82 | 3.96E−05 | 2.90E−04 |
| TRUB1 | 0.82 | 4.06E−03 | 8.52E−03 |
| PTPRS | 0.82 | 2.86E−04 | 1.12E−03 |
| GRWD1 | 0.82 | 1.88E−04 | 8.30E−04 |
| COPS3 | 0.82 | 3.61E−05 | 2.72E−04 |
| UBTF | 0.82 | 1.55E−03 | 4.00E−03 |
| SLC4A7 | 0.82 | 3.64E−03 | 7.79E−03 |
| MRPL14 | 0.82 | 1.88E−03 | 4.63E−03 |
| STMN1 | 0.82 | 4.44E−04 | 1.55E−03 |
| BAG3 | 0.82 | 4.64E−04 | 1.61E−03 |
| PGAM1 | 0.82 | 5.43E−05 | 3.56E−04 |
| ZNF317 | 0.82 | 1.92E−03 | 4.72E−03 |
| AIP | 0.82 | 2.22E−03 | 5.28E−03 |
| FOXP4 | 0.82 | 1.01E−03 | 2.85E−03 |
| IL13RA2 | 0.82 | 1.68E−04 | 7.71E−04 |
| ARMC6 | 0.82 | 4.24E−05 | 3.01E−04 |
| BCLAF1 | 0.82 | 2.79E−05 | 2.33E−04 |
| RBM39 | 0.82 | 5.22E−04 | 1.75E−03 |
| NCBP1 | 0.82 | 6.37E−04 | 2.03E−03 |
| NFAT5 | 0.82 | 4.54E−03 | 9.29E−03 |
| NLE1 | 0.82 | 1.14E−04 | 5.88E−04 |
| CEP350 | 0.82 | 7.69E−04 | 2.32E−03 |
| ZC3H7A | 0.82 | 9.97E−04 | 2.84E−03 |
| AARS2 | 0.82 | 3.53E−03 | 7.63E−03 |
| ZMAT2 | 0.82 | 1.12E−04 | 5.82E−04 |
| MLEC | 0.82 | 1.12E−04 | 5.82E−04 |
| ELAVL1 | 0.82 | 3.70E−04 | 1.36E−03 |
| MRPL12 | 0.82 | 2.94E−03 | 6.60E−03 |
| PSMB2 | 0.82 | 2.06E−04 | 8.82E−04 |
| PCBP2 | 0.82 | 2.45E−05 | 2.13E−04 |
| CHUK | 0.82 | 8.41E−05 | 4.81E−04 |
| TMX4 | 0.82 | 3.93E−05 | 2.89E−04 |
| XPO5 | 0.82 | 9.86E−04 | 2.81E−03 |
| PRAME | 0.82 | 3.32E−04 | 1.25E−03 |
| C19orf54 | 0.81 | 2.73E−03 | 6.22E−03 |
| ELOVL5 | 0.81 | 1.13E−04 | 5.83E−04 |
| POLR2E | 0.81 | 3.32E−03 | 7.27E−03 |
| COPRS | 0.81 | 2.09E−03 | 5.03E−03 |
| FMN1 | 0.81 | 1.49E−03 | 3.87E−03 |
| DDX56 | 0.81 | 2.06E−04 | 8.83E−04 |
| BOP1 | 0.81 | 1.87E−05 | 1.80E−04 |
| PIGO | 0.81 | 4.69E−04 | 1.62E−03 |
| MTA2 | 0.81 | 1.16E−04 | 5.94E−04 |
| PGM2 | 0.81 | 5.40E−04 | 1.80E−03 |
| RASSF1 | 0.81 | 2.19E−04 | 9.20E−04 |
| AKAP8L | 0.81 | 2.25E−04 | 9.36E−04 |
| FARSA | 0.81 | 1.05E−04 | 5.61E−04 |
| MASTL | 0.81 | 1.04E−03 | 2.93E−03 |
| SUN2 | 0.81 | 5.80E−04 | 1.89E−03 |
| WWC2 | 0.81 | 4.39E−03 | 9.05E−03 |
| BAG4 | 0.81 | 3.21E−03 | 7.08E−03 |
| TMEM161A | 0.81 | 2.96E−03 | 6.64E−03 |
| MMADHC | 0.81 | 1.18E−05 | 1.42E−04 |
| NF2 | 0.81 | 6.15E−04 | 1.98E−03 |
| PPP4R2 | 0.81 | 3.48E−04 | 1.29E−03 |
| NDRG3 | 0.81 | 6.13E−05 | 3.88E−04 |
| CNOT6 | 0.81 | 3.43E−03 | 7.45E−03 |
| SPIDR | 0.81 | 3.97E−03 | 8.38E−03 |
| NMRAL1 | 0.81 | 2.57E−03 | 5.93E−03 |
| FTSJ3 | 0.81 | 2.77E−04 | 1.09E−03 |
| EMG1 | 0.81 | 2.36E−04 | 9.70E−04 |
| RBM19 | 0.81 | 4.29E−03 | 8.90E−03 |
| GGCT | 0.81 | 4.22E−03 | 8.77E−03 |
| EIFSB | 0.81 | 1.79E−03 | 4.46E−03 |
| MRPL19 | 0.81 | 1.20E−04 | 6.08E−04 |
| EIF2B3 | 0.81 | 9.30E−04 | 2.68E−03 |
| KAT7 | 0.81 | 7.19E−05 | 4.31E−04 |
| ZNF511 | 0.81 | 1.09E−03 | 3.04E−03 |
| EIF3J | 0.81 | 1.13E−03 | 3.11E−03 |
| PRPF4 | 0.81 | 7.99E−05 | 4.63E−04 |
| NDUFA10 | 0.81 | 4.45E−03 | 9.14E−03 |
| MFAP2 | 0.81 | 1.60E−03 | 4.10E−03 |
| HAX1 | 0.81 | 1.70E−05 | 1.71E−04 |
| GARS | 0.81 | 8.20E−05 | 4.72E−04 |
| CTCF | 0.81 | 1.72E−04 | 7.79E−04 |
| AGPS | 0.81 | 3.75E−06 | 7.16E−05 |
| GTF3C4 | 0.81 | 1.89E−04 | 8.32E−04 |
| FKTN | 0.81 | 2.27E−03 | 5.37E−03 |
| MADD | 0.81 | 3.41E−03 | 7.42E−03 |
| ZW10 | 0.81 | 3.04E−03 | 6.78E−03 |
| MCAT | 0.81 | 1.18E−03 | 3.23E−03 |
| TBL3 | 0.81 | 3.79E−03 | 8.07E−03 |
| UQCRFS1 | 0.81 | 8.93E−04 | 2.59E−03 |
| ARGLU1 | 0.81 | 3.48E−04 | 1.29E−03 |
| TARDBP | 0.81 | 2.40E−05 | 2.10E−04 |
| PSMC6 | 0.81 | 1.08E−04 | 5.68E−04 |
| RHOA | 0.81 | 1.76E−05 | 1.74E−04 |
| IREB2 | 0.81 | 1.31E−03 | 3.50E−03 |
| DDX10 | 0.81 | 2.91E−03 | 6.54E−03 |
| TRIOBP | 0.81 | 3.51E−04 | 1.30E−03 |
| SEMA4C | 0.81 | 3.76E−04 | 1.37E−03 |
| NUDT5 | 0.81 | 3.38E−05 | 2.64E−04 |
| TMTC3 | 0.81 | 5.13E−04 | 1.73E−03 |
| NARS | 0.81 | 4.55E−04 | 1.58E−03 |
| NXF1 | 0.81 | 1.21E−03 | 3.27E−03 |
| PRKDC | 0.81 | 3.96E−06 | 7.38E−05 |
| GADD45GIP1 | 0.81 | 2.00E−03 | 4.86E−03 |
| ZWILCH | 0.81 | 1.01E−03 | 2.85E−03 |
| UBA2 | 0.81 | 1.87E−05 | 1.80E−04 |
| AOR | 0.81 | 7.62E−04 | 2.30E−03 |
| KIF7 | 0.81 | 1.51E−03 | 3.91E−03 |
| IRF2BP1 | 0.81 | 1.04E−04 | 5.58E−04 |
| CCT6A | 0.81 | 1.40E−05 | 1.56E−04 |
| IPO4 | 0.81 | 4.98E−05 | 3.35E−04 |
| NOP56 | 0.81 | 9.90E−05 | 5.39E−04 |
| IWS1 | 0.81 | 3.94E−05 | 2.89E−04 |
| GART | 0.81 | 2.48E−04 | 1.01E−03 |
| RAD21 | 0.81 | 4.32E−06 | 7.81E−05 |
| SUV39H1 | 0.81 | 2.25E−04 | 9.36E−04 |
| ETV6 | 0.81 | 3.83E−03 | 8.13E−03 |
| PHTF1 | 0.81 | 2.81E−03 | 6.37E−03 |
| STOM | 0.81 | 1.98E−05 | 1.87E−04 |
| TCERG1 | 0.81 | 1.19E−03 | 3.25E−03 |
| GFM2 | 0.81 | 2.40E−04 | 9.84E−04 |
| IMPDH2 | 0.81 | 6.80E−05 | 4.17E−04 |
| THYN1 | 0.81 | 4.86E−03 | 9.78E−03 |
| TSTD2 | 0.81 | 6.24E−04 | 2.00E−03 |
| PHTF2 | 0.81 | 1.31E−03 | 3.50E−03 |
| GDI2 | 0.81 | 1.99E−05 | 1.87E−04 |
| ELP6 | 0.81 | 7.71E−04 | 2.32E−03 |
| CNOT2 | 0.81 | 3.38E−03 | 7.38E−03 |
| CTNND1 | 0.81 | 1.76E−05 | 1.74E−04 |
| SLC4A1AP | 0.81 | 2.00E−03 | 4.87E−03 |
| RNASEH1 | 0.81 | 2.40E−04 | 9.82E−04 |
| MATR3 | 0.81 | 1.07E−04 | 5.66E−04 |
| SCO1 | 0.81 | 1.60E−04 | 7.43E−04 |
| FAM208B | 0.81 | 5.58E−05 | 3.63E−04 |
| ZFP36L1 | 0.81 | 1.39E−04 | 6.74E−04 |
| MRPS23 | 0.81 | 1.16E−03 | 3.18E−03 |
| PSME3 | 0.81 | 2.84E−05 | 2.36E−04 |
| SNRNP27 | 0.80 | 7.20E−04 | 2.21E−03 |
| KPNA2 | 0.80 | 2.62E−05 | 2.23E−04 |
| MRPS31 | 0.80 | 8.21E−04 | 2.43E−03 |
| BRIX1 | 0.80 | 1.66E−03 | 4.21E−03 |
| TRIM37 | 0.80 | 1.75E−03 | 4.38E−03 |
| TXNDC5 | 0.80 | 4.61E−05 | 3.21E−04 |
| SNHG7 | 0.80 | 6.19E−04 | 1.99E−03 |
| POC5 | 0.80 | 1.12E−03 | 3.10E−03 |
| MRPS18A | 0.80 | 6.32E−04 | 2.02E−03 |
| ARHGAP17 | 0.80 | 2.64E−04 | 1.05E−03 |
| UBE4B | 0.80 | 2.44E−03 | 5.70E−03 |
| CTNNB1 | 0.80 | 8.37E−06 | 1.15E−04 |
| C15orf57 | 0.80 | 9.22E−04 | 2.66E−03 |
| NUP43 | 0.80 | 4.93E−03 | 9.89E−03 |
| TOMM40 | 0.80 | 3.14E−05 | 2.52E−04 |
| DDX1 | 0.80 | 4.40E−06 | 7.90E−05 |
| ILKAP | 0.80 | 1.64E−03 | 4.17E−03 |
| DIABLO | 0.80 | 2.33E−03 | 5.49E−03 |
| NDUFC2 | 0.80 | 3.38E−04 | 1.26E−03 |
| NOP2 | 0.80 | 3.89E−04 | 1.40E−03 |
| CDK11A | 0.80 | 5.11E−04 | 1.72E−03 |
| LEO1 | 0.80 | 1.34E−03 | 3.57E−03 |
| TIMM10 | 0.80 | 7.19E−04 | 2.21E−03 |
| EDRF1 | 0.80 | 3.75E−03 | 7.99E−03 |
| UGCG | 0.80 | 3.64E−03 | 7.79E−03 |
| UTP4 | 0.80 | 6.92E−04 | 2.16E−03 |
| SF3A1 | 0.80 | 4.16E−05 | 2.99E−04 |
| DPH2 | 0.80 | 6.77E−04 | 2.13E−03 |
| ZBTB11 | 0.80 | 1.79E−03 | 4.46E−03 |
| PPIL3 | 0.80 | 4.78E−04 | 1.65E−03 |
| TAF9 | 0.80 | 4.68E−04 | 1.62E−03 |
| INTS4 | 0.80 | 1.05E−03 | 2.95E−03 |
| AGBL5 | 0.80 | 1.77E−03 | 4.41E−03 |
| RECQL4 | 0.80 | 3.29E−03 | 7.23E−03 |
| CLNS1A | 0.80 | 6.26E−05 | 3.94E−04 |
| IER3 | 0.80 | 1.30E−06 | 4.29E−05 |
| SUPT16H | 0.80 | 8.63E−06 | 1.17E−04 |
| TMCO6 | 0.80 | 2.09E−03 | 5.02E−03 |
| CLPP | 0.80 | 8.42E−04 | 2.48E−03 |
| TOP2B | 0.80 | 3.14E−04 | 1.19E−03 |
| BRMS1 | 0.80 | 2.65E−04 | 1.06E−03 |
| WNK1 | 0.80 | 2.67E−05 | 2.26E−04 |
| ZCCHC3 | 0.80 | 3.82E−03 | 8.12E−03 |
| EIF3B | 0.80 | 1.21E−06 | 4.11E−05 |
| UTP15 | 0.80 | 6.93E−04 | 2.16E−03 |
| WDR75 | 0.80 | 2.73E−05 | 2.29E−04 |
| RFC1 | 0.80 | 2.70E−03 | 6.16E−03 |
| SMARCAS | 0.80 | 4.98E−04 | 1.70E−03 |
| CPSF3 | 0.80 | 3.75E−04 | 1.37E−03 |
| BCAS2 | 0.80 | 4.05E−04 | 1.45E−03 |
| DNAJC9 | 0.80 | 2.70E−03 | 6.16E−03 |
| SNRPB | 0.80 | 1.81E−05 | 1.77E−04 |
| MRPL42 | 0.80 | 4.55E−03 | 9.30E−03 |
| TRIP13 | 0.80 | 1.11E−03 | 3.08E−03 |
| RPL26L1 | 0.80 | 5.04E−04 | 1.71E−03 |
| RBPJ | 0.80 | 3.88E−03 | 8.23E−03 |
| DUSP6 | 0.80 | 1.05E−04 | 5.62E−04 |
| FAM98B | 0.80 | 2.42E−07 | 2.12E−05 |
| ZC3HC1 | 0.80 | 9.86E−04 | 2.81E−03 |
| CNOT7 | 0.80 | 2.69E−05 | 2.27E−04 |
| PES1 | 0.80 | 5.80E−04 | 1.89E−03 |
| NABP2 | 0.80 | 1.31E−03 | 3.51E−03 |
| PCNT | 0.80 | 1.96E−03 | 4.77E−03 |
| LOC102723897 | 0.80 | 3.42E−05 | 2.66E−04 |
| DCUN1D5 | 0.80 | 7.58E−04 | 2.29E−03 |
| EXOSC2 | 0.80 | 1.60E−03 | 4.09E−03 |
| SIPA1L2 | 0.80 | 3.18E−03 | 7.03E−03 |
| RCC1L | 0.80 | 1.99E−04 | 8.61E−04 |
| METAP2 | 0.80 | 1.75E−04 | 7.89E−04 |
| PMPCB | 0.80 | 1.26E−04 | 6.31E−04 |
| MGAT5 | 0.80 | 6.51E−04 | 2.06E−03 |
| C17orf53 | 0.80 | 4.76E−03 | 9.61E−03 |
| RHOT1 | 0.80 | 3.41E−03 | 7.42E−03 |
| PNO1 | 0.80 | 4.35E−03 | 8.98E−03 |
| PSMD3 | 0.80 | 3.51E−04 | 1.30E−03 |
| COMMD2 | 0.80 | 1.46E−04 | 6.96E−04 |
| APIP | 0.80 | 2.90E−03 | 6.54E−03 |
| PHKG2 | 0.80 | 1.17E−03 | 3.20E−03 |
| SIK2 | 0.80 | 8.53E−04 | 2.50E−03 |
| TOMM22 | 0.80 | 3.59E−05 | 2.72E−04 |
| SFSWAP | 0.80 | 4.77E−04 | 1.64E−03 |
| ARL6IP1 | 0.80 | 5.77E−04 | 1.88E−03 |
| DCPS | 0.80 | 2.08E−04 | 8.88E−04 |
| RPA2 | 0.80 | 1.21E−04 | 6.12E−04 |
| PRPF3 | 0.80 | 4.32E−04 | 1.52E−03 |
| MAP2K3 | 0.80 | 1.12E−03 | 3.10E−03 |
| LSM3 | 0.79 | 1.68E−03 | 4.25E−03 |
| MKLN1 | 0.79 | 2.55E−03 | 5.89E−03 |
| PELO | 0.79 | 1.17E−03 | 3.21E−03 |
| MAML3 | 0.79 | 1.48E−03 | 3.85E−03 |
| TMEM123 | 0.79 | 4.95E−04 | 1.69E−03 |
| GLO1 | 0.79 | 1.39E−04 | 6.74E−04 |
| COL4A3BP | 0.79 | 4.63E−03 | 9.42E−03 |
| PRMT7 | 0.79 | 7.20E−04 | 2.21E−03 |
| DDX47 | 0.79 | 3.51E−04 | 1.30E−03 |
| CD44 | 0.79 | 4.50E−05 | 3.14E−04 |
| SMARCAD1 | 0.79 | 6.01E−04 | 1.94E−03 |
| YWHAE | 0.79 | 5.41E−06 | 8.78E−05 |
| PTCD1 | 0.79 | 2.74E−03 | 6.24E−03 |
| ALKBH7 | 0.79 | 1.09E−03 | 3.04E−03 |
| CLN6 | 0.79 | 1.41E−04 | 6.81E−04 |
| LCTL | 0.79 | 1.59E−03 | 4.08E−03 |
| RBM7 | 0.79 | 1.70E−04 | 7.74E−04 |
| IMMT | 0.79 | 1.08E−04 | 5.71E−04 |
| SAP18 | 0.79 | 8.93E−04 | 2.59E−03 |
| ANKH | 0.79 | 1.41E−03 | 3.70E−03 |
| HSPH1 | 0.79 | 8.83E−06 | 1.19E−04 |
| MSANTD2 | 0.79 | 1.21E−04 | 6.12E−04 |
| RNASEH2A | 0.79 | 2.14E−04 | 9.03E−04 |
| NPM1 | 0.79 | 8.22E−06 | 1.14E−04 |
| TCP1 | 0.79 | 2.76E−05 | 2.30E−04 |
| IPO9 | 0.79 | 1.67E−05 | 1.69E−04 |
| MCMBP | 0.79 | 7.46E−05 | 4.43E−04 |
| PHIP | 0.79 | 6.95E−04 | 2.17E−03 |
| GLYR1 | 0.79 | 2.00E−03 | 4.87E−03 |
| SEMA4D | 0.79 | 4.13E−04 | 1.47E−03 |
| TCTN3 | 0.79 | 2.15E−03 | 5.14E−03 |
| PSMA2 | 0.79 | 2.73E−05 | 2.29E−04 |
| ELP5 | 0.79 | 4.98E−04 | 1.70E−03 |
| GBAS | 0.79 | 1.34E−03 | 3.57E−03 |
| MED25 | 0.79 | 3.22E−03 | 7.10E−03 |
| LARP1B | 0.79 | 2.91E−03 | 6.55E−03 |
| CAPN15 | 0.79 | 1.65E−03 | 4.18E−03 |
| ASXL1 | 0.79 | 7.99E−04 | 2.38E−03 |
| SNU13 | 0.79 | 8.13E−04 | 2.41E−03 |
| ACAD9 | 0.79 | 1.51E−03 | 3.90E−03 |
| HIRIP3 | 0.79 | 2.66E−03 | 6.09E−03 |
| HNRNPF | 0.79 | 1.73E−04 | 7.81E−04 |
| TARBP2 | 0.79 | 1.93E−04 | 8.46E−04 |
| HAUS4 | 0.79 | 2.24E−03 | 5.31E−03 |
| CASKIN2 | 0.79 | 6.95E−04 | 2.17E−03 |
| PCMT1 | 0.79 | 3.05E−03 | 6.79E−03 |
| ILF2 | 0.79 | 2.31E−05 | 2.05E−04 |
| C20orf27 | 0.79 | 4.36E−05 | 3.07E−04 |
| ZCCHC11 | 0.79 | 4.74E−03 | 9.59E−03 |
| TIAL1 | 0.79 | 4.34E−04 | 1.52E−03 |
| MRPS26 | 0.79 | 6.89E−04 | 2.15E−03 |
| TMEM183A | 0.79 | 6.85E−05 | 4.19E−04 |
| MRPL16 | 0.79 | 1.89E−04 | 8.32E−04 |
| RXRA | 0.79 | 2.81E−03 | 6.36E−03 |
| SNRPA | 0.79 | 2.08E−04 | 8.89E−04 |
| HNRNPH1 | 0.79 | 3.97E−05 | 2.90E−04 |
| CDK6 | 0.79 | 1.30E−05 | 1.49E−04 |
| ZDHHC7 | 0.79 | 1.84E−04 | 8.15E−04 |
| PPP6C | 0.79 | 2.38E−03 | 5.57E−03 |
| DCTPP1 | 0.79 | 3.77E−03 | 8.02E−03 |
| DHX35 | 0.79 | 2.06E−03 | 4.97E−03 |
| MPP5 | 0.79 | 4.57E−04 | 1.59E−03 |
| DDX39A | 0.79 | 1.50E−04 | 7.10E−04 |
| KIAA1524 | 0.79 | 3.15E−04 | 1.19E−03 |
| FAN1 | 0.79 | 8.17E−04 | 2.42E−03 |
| TCHP | 0.79 | 3.51E−03 | 7.60E−03 |
| CDK12 | 0.79 | 1.85E−04 | 8.20E−04 |
| DST | 0.79 | 1.27E−03 | 3.42E−03 |
| PRPF19 | 0.79 | 3.82E−04 | 1.39E−03 |
| SET | 0.79 | 1.04E−05 | 1.30E−04 |
| RRP1 | 0.79 | 6.56E−05 | 4.07E−04 |
| GUF1 | 0.79 | 2.09E−04 | 8.92E−04 |
| FKBP4 | 0.79 | 4.73E−06 | 8.22E−05 |
| MED20 | 0.79 | 2.42E−03 | 5.65E−03 |
| DNAJC11 | 0.79 | 7.42E−04 | 2.26E−03 |
| NOC3L | 0.79 | 1.13E−03 | 3.11E−03 |
| MRPL38 | 0.79 | 5.19E−04 | 1.74E−03 |
| C1QBP | 0.79 | 2.63E−06 | 5.95E−05 |
| PUM3 | 0.79 | 4.53E−04 | 1.58E−03 |
| PPME1 | 0.79 | 6.34E−04 | 2.03E−03 |
| LSG1 | 0.79 | 1.10E−03 | 3.05E−03 |
| ARHGEF2 | 0.79 | 2.12E−04 | 8.98E−04 |
| RNF166 | 0.79 | 8.34E−04 | 2.46E−03 |
| FAM53C | 0.79 | 7.55E−04 | 2.29E−03 |
| CDCA5 | 0.79 | 1.71E−04 | 7.77E−04 |
| ZNF778 | 0.79 | 4.42E−03 | 9.10E−03 |
| ACOX3 | 0.79 | 6.80E−05 | 4.17E−04 |
| DEK | 0.79 | 4.24E−05 | 3.01E−04 |
| ZFYVE9 | 0.79 | 2.94E−03 | 6.61E−03 |
| POLR1E | 0.79 | 6.08E−06 | 9.46E−05 |
| VWA9 | 0.79 | 1.77E−04 | 7.93E−04 |
| CENPH | 0.79 | 1.96E−03 | 4.78E−03 |
| TUBGCP3 | 0.79 | 9.82E−05 | 5.37E−04 |
| BRD8 | 0.79 | 3.53E−03 | 7.63E−03 |
| MRPS18C | 0.78 | 3.76E−03 | 8.02E−03 |
| ERI3 | 0.78 | 3.52E−03 | 7.61E−03 |
| MAT2A | 0.78 | 1.00E−05 | 1.29E−04 |
| COA7 | 0.78 | 4.25E−04 | 1.50E−03 |
| ASUN | 0.78 | 4.81E−04 | 1.65E−03 |
| UTP14A | 0.78 | 7.16E−04 | 2.21E−03 |
| POLE3 | 0.78 | 4.03E−05 | 2.92E−04 |
| TXNL4B | 0.78 | 1.87E−04 | 8.26E−04 |
| TUBG2 | 0.78 | 3.56E−03 | 7.66E−03 |
| KNSTRN | 0.78 | 1.72E−04 | 7.79E−04 |
| POFUT1 | 0.78 | 3.54E−05 | 2.70E−04 |
| DHX34 | 0.78 | 6.40E−04 | 2.04E−03 |
| RPL7L1 | 0.78 | 4.24E−04 | 1.50E−03 |
| SART3 | 0.78 | 1.74E−03 | 4.35E−03 |
| ITPRIP | 0.78 | 4.52E−04 | 1.57E−03 |
| SLC2A4RG | 0.78 | 1.01E−03 | 2.85E−03 |
| ZRANB1 | 0.78 | 1.68E−03 | 4.25E−03 |
| CCDC124 | 0.78 | 8.09E−04 | 2.40E−03 |
| MRPL47 | 0.78 | 1.63E−03 | 4.15E−03 |
| NUP98 | 0.78 | 1.56E−03 | 4.01E−03 |
| PSMC4 | 0.78 | 3.14E−05 | 2.52E−04 |
| SGTA | 0.78 | 1.29E−04 | 6.40E−04 |
| SH3PXD2A | 0.78 | 2.97E−05 | 2.43E−04 |
| NELFCD | 0.78 | 3.45E−05 | 2.67E−04 |
| SRSF4 | 0.78 | 1.01E−05 | 1.30E−04 |
| LMO4 | 0.78 | 2.49E−04 | 1.01E−03 |
| CTNNBL1 | 0.78 | 6.51E−05 | 4.05E−04 |
| LRP5 | 0.78 | 3.72E−03 | 7.95E−03 |
| ESF1 | 0.78 | 7.69E−04 | 2.32E−03 |
| NRF1 | 0.78 | 4.15E−03 | 8.67E−03 |
| YARS | 0.78 | 6.03E−05 | 3.84E−04 |
| CCT5 | 0.78 | 8.61E−06 | 1.17E−04 |
| PSMG1 | 0.78 | 1.65E−03 | 4.18E−03 |
| LRPPRC | 0.78 | 2.01E−04 | 8.65E−04 |
| ADAM10 | 0.78 | 4.24E−05 | 3.01E−04 |
| HDAC1 | 0.78 | 4.09E−05 | 2.96E−04 |
| WDR5 | 0.78 | 3.57E−04 | 1.32E−03 |
| NOL8 | 0.78 | 4.62E−04 | 1.60E−03 |
| PDAP1 | 0.78 | 2.27E−06 | 5.53E−05 |
| CWC25 | 0.78 | 2.03E−03 | 4.93E−03 |
| SRSF1 | 0.78 | 6.23E−07 | 2.84E−05 |
| NCKIPSD | 0.78 | 3.48E−05 | 2.68E−04 |
| MSMO1 | 0.78 | 1.20E−03 | 3.27E−03 |
| UBAC1 | 0.78 | 1.57E−04 | 7.35E−04 |
| HNRNPUL2-BSCL2 | 0.78 | 2.77E−03 | 6.30E−03 |
| CDC25A | 0.78 | 1.46E−03 | 3.82E−03 |
| LUC7L2 | 0.78 | 1.57E−03 | 4.05E−03 |
| MTA3 | 0.78 | 5.60E−04 | 1.84E−03 |
| TFDP1 | 0.78 | 5.29E−04 | 1.77E−03 |
| PPIE | 0.78 | 3.29E−04 | 1.24E−03 |
| PRKCZ | 0.78 | 6.77E−04 | 2.13E−03 |
| PDSS1 | 0.78 | 3.83E−04 | 1.39E−03 |
| MBD3 | 0.78 | 1.06E−03 | 2.96E−03 |
| CHMP4A | 0.78 | 2.99E−04 | 1.15E−03 |
| NME1 | 0.78 | 2.90E−05 | 2.40E−04 |
| C2CD5 | 0.78 | 3.05E−03 | 6.79E−03 |
| PPP1CC | 0.78 | 7.91E−05 | 4.61E−04 |
| KTN1 | 0.78 | 5.13E−07 | 2.56E−05 |
| RCOR3 | 0.78 | 1.80E−03 | 4.47E−03 |
| SNHG19 | 0.78 | 1.98E−03 | 4.82E−03 |
| WDR74 | 0.78 | 3.68E−05 | 2.76E−04 |
| NDUFA9 | 0.78 | 2.30E−03 | 5.42E−03 |
| CLPX | 0.78 | 6.59E−04 | 2.08E−03 |
| C12orf4 | 0.78 | 1.42E−04 | 6.82E−04 |
| STRAP | 0.78 | 3.74E−05 | 2.79E−04 |
| SLF1 | 0.78 | 3.57E−03 | 7.69E−03 |
| PSMA4 | 0.78 | 1.27E−04 | 6.32E−04 |
| ZNF446 | 0.78 | 1.41E−03 | 3.70E−03 |
| DDX21 | 0.78 | 3.51E−05 | 2.69E−04 |
| PDHX | 0.78 | 1.74E−04 | 7.86E−04 |
| NCAPG2 | 0.78 | 3.45E−03 | 7.49E−03 |
| MSL3 | 0.78 | 7.89E−04 | 2.36E−03 |
| RAD18 | 0.78 | 4.28E−03 | 8.87E−03 |
| TAF15 | 0.78 | 1.72E−05 | 1.72E−04 |
| SMC6 | 0.78 | 2.18E−04 | 9.17E−04 |
| WRAP53 | 0.77 | 8.26E−04 | 2.44E−03 |
| SAAL1 | 0.77 | 8.16E−04 | 2.42E−03 |
| MRPL30 | 0.77 | 1.69E−04 | 7.71E−04 |
| TWISTNB | 0.77 | 8.85E−04 | 2.58E−03 |
| WDR73 | 0.77 | 8.14E−04 | 2.41E−03 |
| TMA7 | 0.77 | 7.66E−06 | 1.10E−04 |
| U2SURP | 0.77 | 4.83E−05 | 3.29E−04 |
| AK2 | 0.77 | 5.21E−04 | 1.75E−03 |
| UVRAG | 0.77 | 2.47E−04 | 1.00E−03 |
| LZTS2 | 0.77 | 2.34E−04 | 9.62E−04 |
| METTL16 | 0.77 | 1.68E−03 | 4.25E−03 |
| IKBKAP | 0.77 | 7.68E−04 | 2.32E−03 |
| PRPF4B | 0.77 | 1.18E−04 | 6.03E−04 |
| COQ4 | 0.77 | 1.68E−03 | 4.25E−03 |
| GORASP1 | 0.77 | 1.76E−04 | 7.92E−04 |
| GSTP1 | 0.77 | 4.43E−05 | 3.10E−04 |
| GFM1 | 0.77 | 3.92E−04 | 1.41E−03 |
| TCTN1 | 0.77 | 4.30E−03 | 8.91E−03 |
| UGP2 | 0.77 | 1.94E−03 | 4.75E−03 |
| TMSB4X | 0.77 | 1.93E−03 | 4.73E−03 |
| GATB | 0.77 | 2.69E−03 | 6.14E−03 |
| PPP5C | 0.77 | 6.94E−05 | 4.23E−04 |
| PRDX6 | 0.77 | 8.46E−06 | 1.16E−04 |
| MRPL2 | 0.77 | 7.57E−04 | 2.29E−03 |
| PPID | 0.77 | 2.52E−03 | 5.85E−03 |
| COPS4 | 0.77 | 1.38E−03 | 3.63E−03 |
| TTC19 | 0.77 | 8.64E−04 | 2.52E−03 |
| POLR1C | 0.77 | 1.19E−03 | 3.25E−03 |
| QTRT1 | 0.77 | 3.35E−04 | 1.25E−03 |
| GGPS1 | 0.77 | 3.85E−04 | 1.39E−03 |
| PTTG1 | 0.77 | 2.22E−03 | 5.28E−03 |
| SNRPB2 | 0.77 | 1.82E−04 | 8.10E−04 |
| CDC26 | 0.77 | 3.99E−03 | 8.41E−03 |
| PATZ1 | 0.77 | 1.70E−03 | 4.27E−03 |
| EFTUD2 | 0.77 | 3.41E−05 | 2.66E−04 |
| SMNDC1 | 0.77 | 4.31E−03 | 8.92E−03 |
| CSTF3 | 0.77 | 2.96E−04 | 1.14E−03 |
| LPCAT1 | 0.77 | 1.15E−03 | 3.17E−03 |
| GTF3C6 | 0.77 | 2.52E−03 | 5.85E−03 |
| CDK10 | 0.77 | 3.75E−03 | 8.00E−03 |
| THAP9-AS1 | 0.77 | 3.59E−03 | 7.73E−03 |
| SRSF6 | 0.77 | 2.79E−04 | 1.09E−03 |
| CD70 | 0.77 | 9.31E−04 | 2.68E−03 |
| DSN1 | 0.77 | 2.53E−03 | 5.85E−03 |
| PHF5A | 0.77 | 1.43E−04 | 6.85E−04 |
| CLASP2 | 0.77 | 4.52E−05 | 3.15E−04 |
| GTF3C3 | 0.77 | 1.18E−03 | 3.22E−03 |
| ADH5 | 0.77 | 7.37E−04 | 2.25E−03 |
| ASNSD1 | 0.77 | 3.51E−03 | 7.59E−03 |
| TMEM63A | 0.77 | 2.13E−04 | 9.00E−04 |
| TACO1 | 0.77 | 3.01E−03 | 6.72E−03 |
| LATS2 | 0.77 | 2.55E−03 | 5.89E−03 |
| FGFR1OP2 | 0.77 | 3.98E−03 | 8.40E−03 |
| PACSIN2 | 0.77 | 2.23E−03 | 5.29E−03 |
| TSN | 0.77 | 1.34E−03 | 3.57E−03 |
| AK6 | 0.77 | 7.29E−04 | 2.23E−03 |
| PSIP1 | 0.77 | 1.36E−04 | 6.66E−04 |
| ENKD1 | 0.77 | 6.88E−04 | 2.15E−03 |
| IDH3B | 0.77 | 4.78E−05 | 3.27E−04 |
| ETV5 | 0.77 | 1.08E−03 | 3.01E−03 |
| SH2B3 | 0.77 | 1.54E−04 | 7.26E−04 |
| KDM4A | 0.77 | 2.04E−04 | 8.74E−04 |
| ARL3 | 0.77 | 6.20E−04 | 1.99E−03 |
| UBE2S | 0.77 | 7.52E−05 | 4.45E−04 |
| SRSF11 | 0.77 | 5.40E−05 | 3.55E−04 |
| PODXL | 0.77 | 1.38E−03 | 3.63E−03 |
| MRPL21 | 0.77 | 7.71E−04 | 2.32E−03 |
| DPYSL3 | 0.77 | 1.64E−03 | 4.17E−03 |
| ATF7IP | 0.77 | 4.23E−03 | 8.80E−03 |
| ZNF217 | 0.77 | 3.88E−05 | 2.86E−04 |
| PNMAL1 | 0.77 | 3.29E−03 | 7.22E−03 |
| GRAMD1B | 0.77 | 9.84E−05 | 5.37E−04 |
| MTF2 | 0.77 | 1.94E−04 | 8.48E−04 |
| ACIN1 | 0.77 | 2.56E−04 | 1.03E−03 |
| BUD31 | 0.76 | 1.59E−05 | 1.67E−04 |
| EPB41L2 | 0.76 | 7.53E−04 | 2.28E−03 |
| COA6 | 0.76 | 8.09E−04 | 2.40E−03 |
| RBMX | 0.76 | 3.65E−06 | 7.05E−05 |
| KRR1 | 0.76 | 1.03E−03 | 2.90E−03 |
| KIAA0753 | 0.76 | 4.19E−03 | 8.73E−03 |
| SNX2 | 0.76 | 8.49E−04 | 2.49E−03 |
| ZNF330 | 0.76 | 6.87E−04 | 2.15E−03 |
| IFRD2 | 0.76 | 1.03E−04 | 5.54E−04 |
| UPF3B | 0.76 | 1.28E−03 | 3.43E−03 |
| NOP14 | 0.76 | 3.60E−05 | 2.72E−04 |
| METTL12 | 0.76 | 4.52E−03 | 9.25E−03 |
| PTMS | 0.76 | 9.52E−05 | 5.27E−04 |
| PNRC2 | 0.76 | 1.85E−04 | 8.20E−04 |
| SSBP1 | 0.76 | 2.74E−05 | 2.29E−04 |
| PEX5 | 0.76 | 2.59E−04 | 1.04E−03 |
| HSPA5 | 0.76 | 1.62E−06 | 4.75E−05 |
| SREBF2 | 0.76 | 1.69E−06 | 4.78E−05 |
| RBM23 | 0.76 | 7.20E−05 | 4.31E−04 |
| SNHG17 | 0.76 | 9.29E−04 | 2.68E−03 |
| COX11 | 0.76 | 3.98E−03 | 8.40E−03 |
| IDE | 0.76 | 3.01E−04 | 1.15E−03 |
| SRGAP2C | 0.76 | 2.35E−03 | 5.52E−03 |
| PDCD4 | 0.76 | 3.11E−04 | 1.18E−03 |
| SNRPE | 0.76 | 1.21E−04 | 6.12E−04 |
| TAF11 | 0.76 | 1.13E−03 | 3.13E−03 |
| DHX9 | 0.76 | 7.11E−06 | 1.05E−04 |
| DNMT1 | 0.76 | 9.23E−06 | 1.22E−04 |
| GPATCH4 | 0.76 | 1.77E−04 | 7.93E−04 |
| CAD | 0.76 | 4.03E−05 | 2.92E−04 |
| WDR36 | 0.76 | 7.18E−04 | 2.21E−03 |
| CYB5D1 | 0.76 | 4.64E−03 | 9.42E−03 |
| RBM26 | 0.76 | 3.88E−04 | 1.40E−03 |
| JMJD8 | 0.76 | 2.16E−04 | 9.10E−04 |
| EXOSC1 | 0.76 | 4.11E−03 | 8.60E−03 |
| H1FX | 0.76 | 9.86E−06 | 1.28E−04 |
| GEMIN5 | 0.76 | 4.92E−04 | 1.68E−03 |
| UTP18 | 0.76 | 7.20E−04 | 2.21E−03 |
| NAA25 | 0.76 | 1.93E−03 | 4.73E−03 |
| TMEM97 | 0.76 | 1.61E−03 | 4.10E−03 |
| INTS6 | 0.76 | 5.00E−04 | 1.70E−03 |
| DDX11 | 0.76 | 1.60E−03 | 4.09E−03 |
| RAE1 | 0.76 | 2.63E−05 | 2.24E−04 |
| UIMC1 | 0.76 | 3.36E−03 | 7.34E−03 |
| ZNF593 | 0.76 | 4.23E−03 | 8.80E−03 |
| PTCD2 | 0.76 | 1.55E−03 | 3.99E−03 |
| PGD | 0.76 | 2.23E−06 | 5.50E−05 |
| LMNA | 0.76 | 4.81E−06 | 8.31E−05 |
| NAE1 | 0.76 | 1.55E−03 | 3.99E−03 |
| BCL2L2-PABPN1 | 0.76 | 1.28E−03 | 3.44E−03 |
| PPIH | 0.76 | 3.36E−03 | 7.35E−03 |
| SNX1 | 0.76 | 4.01E−04 | 1.44E−03 |
| PRPF31 | 0.76 | 1.56E−04 | 7.33E−04 |
| SYDE1 | 0.76 | 1.83E−04 | 8.13E−04 |
| DDX51 | 0.76 | 3.88E−05 | 2.86E−04 |
| NUP205 | 0.76 | 1.75E−05 | 1.74E−04 |
| PTMA | 0.76 | 4.59E−06 | 8.09E−05 |
| TUBGCP4 | 0.76 | 6.57E−04 | 2.08E−03 |
| TIMELESS | 0.76 | 2.45E−04 | 1.00E−03 |
| SMC3 | 0.76 | 3.91E−04 | 1.41E−03 |
| XYLB | 0.76 | 1.22E−03 | 3.31E−03 |
| APITD1 | 0.76 | 3.34E−03 | 7.32E−03 |
| ABCF2 | 0.76 | 3.00E−04 | 1.15E−03 |
| NAP1L1 | 0.76 | 6.60E−06 | 9.90E−05 |
| PARN | 0.76 | 5.14E−04 | 1.73E−03 |
| CKAP5 | 0.76 | 1.01E−04 | 5.48E−04 |
| ZNF608 | 0.76 | 3.49E−05 | 2.68E−04 |
| HNRNPM | 0.76 | 1.32E−05 | 1.51E−04 |
| MTERF3 | 0.76 | 7.08E−04 | 2.19E−03 |
| SF3B3 | 0.76 | 2.47E−05 | 2.14E−04 |
| MTMR2 | 0.76 | 1.66E−04 | 7.65E−04 |
| SFPQ | 0.76 | 1.42E−04 | 6.82E−04 |
| USP28 | 0.76 | 1.65E−04 | 7.63E−04 |
| HMGCR | 0.75 | 2.06E−05 | 1.91E−04 |
| NDST1 | 0.75 | 1.39E−05 | 1.56E−04 |
| MY019 | 0.75 | 6.81E−04 | 2.13E−03 |
| AKR7A2 | 0.75 | 1.81E−04 | 8.06E−04 |
| DZIP1 | 0.75 | 2.98E−04 | 1.15E−03 |
| ZNF266 | 0.75 | 3.65E−03 | 7.82E−03 |
| GTF2I | 0.75 | 2.10E−04 | 8.95E−04 |
| NPM3 | 0.75 | 2.33E−04 | 9.59E−04 |
| LINC01029 | 0.75 | 1.73E−03 | 4.34E−03 |
| ZNF692 | 0.75 | 9.05E−04 | 2.62E−03 |
| SNHG15 | 0.75 | 1.25E−03 | 3.38E−03 |
| ASAP1 | 0.75 | 3.50E−03 | 7.57E−03 |
| VPS37B | 0.75 | 1.09E−04 | 5.75E−04 |
| ATP5F1 | 0.75 | 5.20E−05 | 3.46E−04 |
| HEATR1 | 0.75 | 1.58E−05 | 1.66E−04 |
| N4BP2L2 | 0.75 | 3.84E−03 | 8.14E−03 |
| STIL | 0.75 | 9.90E−04 | 2.82E−03 |
| ARHGAP29 | 0.75 | 2.65E−03 | 6.07E−03 |
| SNRPG | 0.75 | 5.58E−04 | 1.84E−03 |
| PRMT1 | 0.75 | 4.62E−07 | 2.47E−05 |
| ANKRD54 | 0.75 | 1.78E−04 | 7.97E−04 |
| SRSF2 | 0.75 | 3.52E−05 | 2.70E−04 |
| NOL11 | 0.75 | 5.77E−04 | 1.88E−03 |
| PRKACB | 0.75 | 4.33E−03 | 8.95E−03 |
| LINC00998 | 0.75 | 1.72E−03 | 4.32E−03 |
| GLI3 | 0.75 | 3.34E−04 | 1.25E−03 |
| GNPTAB | 0.75 | 2.33E−05 | 2.06E−04 |
| U2AF1 | 0.75 | 7.08E−04 | 2.19E−03 |
| DDX46 | 0.75 | 8.62E−04 | 2.52E−03 |
| MRPS15 | 0.75 | 3.36E−04 | 1.26E−03 |
| LYAR | 0.75 | 1.00E−05 | 1.29E−04 |
| WDR18 | 0.75 | 1.59E−04 | 7.41E−04 |
| AMOTL2 | 0.75 | 1.02E−05 | 1.30E−04 |
| NFYB | 0.75 | 1.77E−03 | 4.42E−03 |
| MTHFD1L | 0.75 | 4.87E−04 | 1.67E−03 |
| EIF2S2 | 0.75 | 7.23E−05 | 4.32E−04 |
| GLT8D1 | 0.75 | 1.05E−04 | 5.61E−04 |
| MCCC1 | 0.75 | 4.37E−04 | 1.53E−03 |
| RRP1B | 0.75 | 2.70E−04 | 1.07E−03 |
| MARS | 0.75 | 8.90E−05 | 5.01E−04 |
| C3orf14 | 0.75 | 2.81E−04 | 1.10E−03 |
| CCP110 | 0.75 | 9.08E−04 | 2.62E−03 |
| CENPE | 0.75 | 4.94E−04 | 1.69E−03 |
| DYNLL1 | 0.75 | 2.11E−06 | 5.36E−05 |
| ARHGAP5 | 0.75 | 2.02E−03 | 4.89E−03 |
| NPRL3 | 0.75 | 2.22E−04 | 9.26E−04 |
| RBBP4 | 0.75 | 2.81E−05 | 2.34E−04 |
| PTGES3 | 0.75 | 4.07E−05 | 2.95E−04 |
| PLEKHAS | 0.75 | 3.51E−03 | 7.59E−03 |
| C1orf112 | 0.75 | 4.51E−04 | 1.57E−03 |
| PYCR1 | 0.75 | 8.77E−04 | 2.56E−03 |
| ATAD2 | 0.75 | 4.06E−06 | 7.53E−05 |
| NUDT21 | 0.75 | 1.97E−04 | 8.55E−04 |
| MLH1 | 0.75 | 9.08E−04 | 2.62E−03 |
| CCDC86 | 0.75 | 6.90E−05 | 4.21E−04 |
| TAF2 | 0.75 | 1.86E−06 | 4.95E−05 |
| SPATS2 | 0.75 | 7.47E−04 | 2.27E−03 |
| SLC25A20 | 0.75 | 2.34E−03 | 5.51E−03 |
| MRPL33 | 0.75 | 1.13E−04 | 5.84E−04 |
| CAMK4 | 0.75 | 2.79E−03 | 6.32E−03 |
| JAGN1 | 0.75 | 2.18E−03 | 5.20E−03 |
| COPS5 | 0.75 | 3.65E−04 | 1.34E−03 |
| OSER1 | 0.74 | 1.17E−03 | 3.20E−03 |
| TMEM50B | 0.74 | 2.42E−04 | 9.90E−04 |
| MTHFD2 | 0.74 | 1.33E−03 | 3.54E−03 |
| PRIM2 | 0.74 | 1.17E−03 | 3.20E−03 |
| RPP30 | 0.74 | 7.20E−04 | 2.21E−03 |
| RBM12 | 0.74 | 3.16E−05 | 2.53E−04 |
| KCTD20 | 0.74 | 3.40E−04 | 1.27E−03 |
| CYTH1 | 0.74 | 1.93E−03 | 4.73E−03 |
| SRSF3 | 0.74 | 3.23E−05 | 2.56E−04 |
| ERBB2 | 0.74 | 1.76E−05 | 1.74E−04 |
| WBP11 | 0.74 | 1.45E−04 | 6.94E−04 |
| PNN | 0.74 | 6.32E−04 | 2.02E−03 |
| LENG8 | 0.74 | 3.23E−04 | 1.22E−03 |
| NCL | 0.74 | 4.82E−07 | 2.51E−05 |
| IFT122 | 0.74 | 6.24E−04 | 2.00E−03 |
| LAS1L | 0.74 | 1.93E−04 | 8.46E−04 |
| CENPO | 0.74 | 4.06E−03 | 8.51E−03 |
| LINC01578 | 0.74 | 5.97E−04 | 1.93E−03 |
| POLA1 | 0.74 | 1.20E−03 | 3.26E−03 |
| MGLL | 0.74 | 3.94E−05 | 2.89E−04 |
| EXOG | 0.74 | 1.26E−03 | 3.39E−03 |
| BTAF1 | 0.74 | 6.48E−04 | 2.06E−03 |
| B3GALNT2 | 0.74 | 2.49E−03 | 5.79E−03 |
| GPN3 | 0.74 | 2.93E−03 | 6.59E−03 |
| KIF14 | 0.74 | 6.70E−04 | 2.11E−03 |
| MED27 | 0.74 | 4.95E−04 | 1.69E−03 |
| CHAF1A | 0.74 | 5.27E−04 | 1.76E−03 |
| FAM111B | 0.74 | 3.09E−03 | 6.87E−03 |
| TCEA2 | 0.74 | 1.03E−04 | 5.54E−04 |
| INTS8 | 0.74 | 4.23E−03 | 8.80E−03 |
| PCNX1 | 0.74 | 4.19E−03 | 8.74E−03 |
| CDKN2C | 0.74 | 5.10E−04 | 1.72E−03 |
| RAD51C | 0.74 | 6.51E−04 | 2.06E−03 |
| TNPO1 | 0.74 | 1.45E−05 | 1.58E−04 |
| APEX1 | 0.74 | 2.65E−04 | 1.06E−03 |
| TRMT61B | 0.74 | 2.08E−03 | 5.01E−03 |
| KHSRP | 0.74 | 2.58E−07 | 2.14E−05 |
| SNRNP70 | 0.74 | 4.68E−05 | 3.24E−04 |
| CBX6 | 0.74 | 3.04E−04 | 1.16E−03 |
| NUP133 | 0.74 | 6.20E−06 | 9.53E−05 |
| RILPL2 | 0.74 | 7.90E−04 | 2.37E−03 |
| MDC1 | 0.74 | 7.38E−04 | 2.25E−03 |
| IFT22 | 0.74 | 2.59E−05 | 2.21E−04 |
| TULP3 | 0.74 | 1.88E−04 | 8.27E−04 |
| ADCK4 | 0.74 | 1.83E−03 | 4.51E−03 |
| TRAP1 | 0.74 | 3.08E−04 | 1.18E−03 |
| MRTO4 | 0.74 | 1.87E−05 | 1.80E−04 |
| ECD | 0.74 | 4.35E−03 | 8.98E−03 |
| RANBP1 | 0.74 | 3.79E−04 | 1.38E−03 |
| DARS2 | 0.74 | 3.87E−04 | 1.40E−03 |
| MRPL1 | 0.74 | 1.86E−04 | 8.25E−04 |
| IDI1 | 0.74 | 1.65E−03 | 4.18E−03 |
| HAUSS | 0.74 | 8.29E−04 | 2.45E−03 |
| CDKN2AIPNL | 0.74 | 6.55E−04 | 2.07E−03 |
| MAGOH | 0.74 | 1.16E−03 | 3.19E−03 |
| SNRPD1 | 0.74 | 6.38E−05 | 3.99E−04 |
| SOX11 | 0.74 | 1.85E−05 | 1.79E−04 |
| SKIV2L2 | 0.74 | 1.97E−05 | 1.86E−04 |
| DDX19B | 0.74 | 5.14E−04 | 1.73E−03 |
| NAT10 | 0.74 | 5.56E−05 | 3.62E−04 |
| TRIP6 | 0.74 | 5.11E−04 | 1.72E−03 |
| EPHA2 | 0.74 | 6.25E−06 | 9.58E−05 |
| TDRD3 | 0.74 | 2.85E−03 | 6.43E−03 |
| SS18L2 | 0.74 | 3.90E−03 | 8.25E−03 |
| SUZ12 | 0.74 | 1.91E−04 | 8.39E−04 |
| ARHGAP12 | 0.74 | 7.65E−05 | 4.50E−04 |
| PPP1R7 | 0.74 | 3.17E−04 | 1.20E−03 |
| USP1 | 0.74 | 6.27E−05 | 3.94E−04 |
| NUP107 | 0.74 | 1.20E−03 | 3.27E−03 |
| ADGRL1 | 0.74 | 5.51E−04 | 1.82E−03 |
| COQ7 | 0.73 | 9.09E−04 | 2.62E−03 |
| NARS2 | 0.73 | 1.74E−03 | 4.37E−03 |
| PTPN3 | 0.73 | 3.09E−03 | 6.87E−03 |
| NRSN2 | 0.73 | 1.05E−04 | 5.61E−04 |
| ENG | 0.73 | 1.25E−04 | 6.27E−04 |
| EXOSC3 | 0.73 | 3.88E−04 | 1.40E−03 |
| ELMSAN1 | 0.73 | 1.62E−05 | 1.67E−04 |
| PCDH1 | 0.73 | 1.41E−05 | 1.57E−04 |
| THOC2 | 0.73 | 3.19E−05 | 2.54E−04 |
| DNAJB11 | 0.73 | 1.82E−04 | 8.08E−04 |
| NME7 | 0.73 | 1.36E−03 | 3.61E−03 |
| ALDH1B1 | 0.73 | 7.06E−05 | 4.26E−04 |
| EEF1AKMT1 | 0.73 | 4.63E−03 | 9.42E−03 |
| ST3GAL4 | 0.73 | 2.18E−05 | 1.98E−04 |
| NAA15 | 0.73 | 8.17E−06 | 1.14E−04 |
| PCAT7 | 0.73 | 4.45E−03 | 9.14E−03 |
| MAD2L1 | 0.73 | 6.05E−05 | 3.85E−04 |
| AP2S1 | 0.73 | 1.91E−05 | 1.82E−04 |
| RFX1 | 0.73 | 2.27E−04 | 9.40E−04 |
| ZRANB2 | 0.73 | 1.02E−03 | 2.87E−03 |
| POP4 | 0.73 | 4.76E−04 | 1.64E−03 |
| HNRNPA2B1 | 0.73 | 2.62E−05 | 2.23E−04 |
| XPO4 | 0.73 | 1.50E−03 | 3.90E−03 |
| CMTM7 | 0.73 | 7.25E−05 | 4.33E−04 |
| NOP16 | 0.73 | 2.63E−04 | 1.05E−03 |
| HSPA9 | 0.73 | 2.47E−06 | 5.82E−05 |
| EX01 | 0.73 | 1.63E−04 | 7.52E−04 |
| RIOK1 | 0.73 | 1.96E−03 | 4.78E−03 |
| WDR12 | 0.73 | 1.47E−03 | 3.83E−03 |
| INTS12 | 0.73 | 2.00E−04 | 8.64E−04 |
| LUC7L | 0.73 | 2.76E−04 | 1.08E−03 |
| TIA1 | 0.73 | 4.17E−04 | 1.48E−03 |
| ETAA1 | 0.73 | 1.30E−05 | 1.49E−04 |
| NUP93 | 0.73 | 5.31E−06 | 8.71E−05 |
| LARS | 0.73 | 2.70E−06 | 6.04E−05 |
| MAD2L2 | 0.73 | 4.36E−04 | 1.53E−03 |
| HMCES | 0.73 | 7.67E−04 | 2.32E−03 |
| SDHD | 0.73 | 7.52E−04 | 2.28E−03 |
| PPP2R4 | 0.73 | 1.36E−05 | 1.54E−04 |
| SRSF7 | 0.73 | 5.77E−05 | 3.72E−04 |
| MKNK2 | 0.73 | 5.17E−04 | 1.74E−03 |
| SF3A3 | 0.73 | 9.50E−05 | 5.26E−04 |
| H2AFV | 0.73 | 2.00E−05 | 1.87E−04 |
| SEH1L | 0.73 | 4.80E−05 | 3.27E−04 |
| SAPCD2 | 0.73 | 1.93E−03 | 4.72E−03 |
| TGS1 | 0.73 | 6.97E−05 | 4.24E−04 |
| PSPC1 | 0.73 | 7.92E−05 | 4.61E−04 |
| NAA38 | 0.73 | 4.22E−04 | 1.49E−03 |
| FJX1 | 0.73 | 1.69E−03 | 4.25E−03 |
| CBX1 | 0.73 | 5.23E−04 | 1.75E−03 |
| MKKS | 0.73 | 2.75E−03 | 6.25E−03 |
| ACP6 | 0.73 | 1.10E−03 | 3.04E−03 |
| PCDHA6 | 0.73 | 4.18E−03 | 8.71E−03 |
| RAPGEF6 | 0.72 | 1.19E−03 | 3.25E−03 |
| DTYMK | 0.72 | 7.25E−04 | 2.22E−03 |
| MRPL13 | 0.72 | 1.19E−04 | 6.04E−04 |
| UBXN2A | 0.72 | 4.04E−04 | 1.45E−03 |
| NACC1 | 0.72 | 1.15E−05 | 1.40E−04 |
| C16orf59 | 0.72 | 4.21E−03 | 8.77E−03 |
| MBNL1 | 0.72 | 1.89E−04 | 8.32E−04 |
| SNRNP40 | 0.72 | 9.99E−06 | 1.29E−04 |
| FAM126A | 0.72 | 4.02E−03 | 8.45E−03 |
| ECSIT | 0.72 | 6.10E−05 | 3.87E−04 |
| MCM5 | 0.72 | 2.49E−04 | 1.01E−03 |
| OSGEP | 0.72 | 7.63E−04 | 2.31E−03 |
| PRPF38A | 0.72 | 2.11E−04 | 8.97E−04 |
| THOC1 | 0.72 | 1.62E−03 | 4.13E−03 |
| CYP51A1 | 0.72 | 1.30E−04 | 6.42E−04 |
| CENPL | 0.72 | 1.45E−03 | 3.78E−03 |
| TIMM9 | 0.72 | 3.79E−03 | 8.06E−03 |
| FUBP1 | 0.72 | 2.59E−04 | 1.04E−03 |
| KHDRBS3 | 0.72 | 1.84E−03 | 4.54E−03 |
| LIMA1 | 0.72 | 5.53E−04 | 1.83E−03 |
| AIMP2 | 0.72 | 6.23E−05 | 3.92E−04 |
| DNAAF5 | 0.72 | 7.08E−05 | 4.27E−04 |
| PGAM5 | 0.72 | 2.15E−05 | 1.96E−04 |
| CHSY1 | 0.72 | 1.74E−04 | 7.86E−04 |
| GNL3 | 0.72 | 1.55E−04 | 7.28E−04 |
| METTL17 | 0.72 | 7.73E−04 | 2.33E−03 |
| ZNF146 | 0.72 | 1.20E−04 | 6.09E−04 |
| NOM1 | 0.72 | 1.24E−03 | 3.36E−03 |
| PCGF6 | 0.72 | 3.36E−04 | 1.26E−03 |
| CDK2 | 0.72 | 5.18E−04 | 1.74E−03 |
| ING3 | 0.72 | 1.29E−03 | 3.46E−03 |
| CALM3 | 0.72 | 3.66E−06 | 7.07E−05 |
| EIF4A3 | 0.72 | 2.92E−05 | 2.41E−04 |
| POLE | 0.72 | 2.22E−03 | 5.28E−03 |
| TSPAN4 | 0.72 | 1.57E−03 | 4.05E−03 |
| CEP85 | 0.72 | 2.14E−04 | 9.04E−04 |
| APOBEC3G | 0.72 | 2.98E−03 | 6.66E−03 |
| FUS | 0.72 | 1.39E−05 | 1.56E−04 |
| LYRM1 | 0.72 | 3.48E−04 | 1.29E−03 |
| DBF4 | 0.72 | 1.11E−03 | 3.07E−03 |
| PKP4 | 0.72 | 6.59E−04 | 2.08E−03 |
| NUP88 | 0.72 | 1.67E−07 | 1.75E−05 |
| CDT1 | 0.72 | 1.49E−04 | 7.09E−04 |
| TTC27 | 0.72 | 2.82E−04 | 1.10E−03 |
| NEDD4 | 0.72 | 5.89E−04 | 1.91E−03 |
| LTV1 | 0.72 | 1.07E−03 | 3.00E−03 |
| DDX55 | 0.72 | 1.80E−03 | 4.47E−03 |
| IQSEC1 | 0.72 | 9.04E−05 | 5.07E−04 |
| PRDX3 | 0.72 | 2.32E−05 | 2.06E−04 |
| FAM133B | 0.72 | 1.94E−04 | 8.48E−04 |
| RBMXL1 | 0.71 | 7.86E−05 | 4.59E−04 |
| NUP85 | 0.71 | 7.96E−04 | 2.38E−03 |
| CSE1L | 0.71 | 2.73E−05 | 2.29E−04 |
| MTRR | 0.71 | 5.76E−04 | 1.88E−03 |
| GGA2 | 0.71 | 3.45E−04 | 1.28E−03 |
| TAPT1 | 0.71 | 8.77E−04 | 2.56E−03 |
| THEM4 | 0.71 | 1.81E−03 | 4.48E−03 |
| HSPE1 | 0.71 | 1.41E−04 | 6.81E−04 |
| SUPV3L1 | 0.71 | 5.74E−04 | 1.88E−03 |
| SNUPN | 0.71 | 3.76E−04 | 1.37E−03 |
| ALDH18A1 | 0.71 | 3.73E−05 | 2.79E−04 |
| SLC25A25 | 0.71 | 1.12E−04 | 5.82E−04 |
| PA2G4 | 0.71 | 1.64E−06 | 4.75E−05 |
| RRM1 | 0.71 | 2.05E−04 | 8.79E−04 |
| GABPA | 0.71 | 3.38E−04 | 1.26E−03 |
| TECR | 0.71 | 5.21E−04 | 1.75E−03 |
| PKMYT1 | 0.71 | 3.60E−03 | 7.74E−03 |
| SUV39H2 | 0.71 | 8.40E−04 | 2.47E−03 |
| NEAT1 | 0.71 | 2.18E−03 | 5.21E−03 |
| KDM1A | 0.71 | 5.41E−05 | 3.55E−04 |
| MRPL4 | 0.71 | 8.11E−04 | 2.41E−03 |
| NUDCD2 | 0.71 | 3.04E−04 | 1.16E−03 |
| USP39 | 0.71 | 1.72E−04 | 7.79E−04 |
| AARSD1 | 0.71 | 2.52E−04 | 1.02E−03 |
| GTPBP6 | 0.71 | 1.02E−03 | 2.88E−03 |
| SMC4 | 0.71 | 1.37E−04 | 6.69E−04 |
| PUS7 | 0.71 | 3.05E−04 | 1.17E−03 |
| SLC5A6 | 0.71 | 9.86E−05 | 5.37E−04 |
| SHISA4 | 0.71 | 1.94E−04 | 8.48E−04 |
| ILF3 | 0.71 | 2.68E−06 | 6.02E−05 |
| ZNF274 | 0.71 | 5.33E−04 | 1.78E−03 |
| PNPT1 | 0.71 | 3.21E−03 | 7.10E−03 |
| NTMT1 | 0.71 | 2.32E−04 | 9.58E−04 |
| NCAPG | 0.71 | 1.54E−06 | 4.67E−05 |
| ZNF362 | 0.71 | 1.05E−03 | 2.95E−03 |
| TIMM50 | 0.71 | 1.03E−04 | 5.55E−04 |
| CTSC | 0.71 | 2.03E−05 | 1.89E−04 |
| FOXRED2 | 0.71 | 3.51E−04 | 1.30E−03 |
| KPTN | 0.71 | 2.12E−04 | 8.98E−04 |
| CCNL1 | 0.71 | 7.32E−04 | 2.24E−03 |
| MRPL11 | 0.71 | 1.35E−03 | 3.58E−03 |
| FCF1 | 0.71 | 3.86E−04 | 1.40E−03 |
| ASF1B | 0.71 | 8.55E−05 | 4.87E−04 |
| RBL1 | 0.71 | 1.26E−03 | 3.39E−03 |
| TRA2B | 0.71 | 2.97E−05 | 2.43E−04 |
| HSPD1 | 0.71 | 4.48E−05 | 3.13E−04 |
| GPD2 | 0.71 | 2.21E−04 | 9.24E−04 |
| PFN2 | 0.71 | 7.50E−05 | 4.44E−04 |
| KIF20B | 0.71 | 6.27E−04 | 2.01E−03 |
| SLC25A33 | 0.71 | 2.04E−03 | 4.95E−03 |
| ANAPC7 | 0.71 | 2.21E−04 | 9.25E−04 |
| FBXO5 | 0.71 | 4.27E−04 | 1.51E−03 |
| SNRNP25 | 0.71 | 3.18E−05 | 2.54E−04 |
| PAXBP1 | 0.71 | 3.87E−04 | 1.40E−03 |
| CENPN | 0.71 | 1.78E−03 | 4.43E−03 |
| KNTC1 | 0.71 | 1.98E−03 | 4.81E−03 |
| ABTB2 | 0.71 | 6.57E−05 | 4.07E−04 |
| XPNPEP3 | 0.71 | 1.09E−03 | 3.02E−03 |
| GCSH | 0.70 | 1.37E−03 | 3.62E−03 |
| IFT52 | 0.70 | 4.52E−04 | 1.58E−03 |
| RND3 | 0.70 | 3.90E−04 | 1.40E−03 |
| GPSM3 | 0.70 | 7.69E−04 | 2.32E−03 |
| RUVBL1 | 0.70 | 1.02E−05 | 1.30E−04 |
| SPARC | 0.70 | 6.13E−07 | 2.83E−05 |
| HNRNPH3 | 0.70 | 1.45E−04 | 6.91E−04 |
| SRRD | 0.70 | 5.90E−04 | 1.91E−03 |
| MPDZ | 0.70 | 9.07E−04 | 2.62E−03 |
| CWF19L1 | 0.70 | 2.56E−04 | 1.03E−03 |
| RPIA | 0.70 | 4.44E−03 | 9.14E−03 |
| TYMS | 0.70 | 5.10E−05 | 3.40E−04 |
| THADA | 0.70 | 3.66E−03 | 7.82E−03 |
| KBTBD6 | 0.70 | 1.03E−03 | 2.90E−03 |
| STAG1 | 0.70 | 1.65E−03 | 4.18E−03 |
| CASC5 | 0.70 | 5.18E−05 | 3.44E−04 |
| FAM171A1 | 0.70 | 1.35E−03 | 3.58E−03 |
| HJURP | 0.70 | 7.92E−05 | 4.61E−04 |
| RBM17 | 0.70 | 1.50E−04 | 7.12E−04 |
| FADS1 | 0.70 | 4.08E−05 | 2.95E−04 |
| PCNA | 0.70 | 1.04E−05 | 1.30E−04 |
| PROSER3 | 0.70 | 1.84E−03 | 4.54E−03 |
| RITA1 | 0.70 | 6.61E−05 | 4.08E−04 |
| KIFC1 | 0.70 | 4.98E−05 | 3.35E−04 |
| NCAPD3 | 0.70 | 7.77E−06 | 1.11E−04 |
| FHL3 | 0.70 | 3.13E−04 | 1.19E−03 |
| POLR3B | 0.70 | 2.93E−04 | 1.13E−03 |
| NUDT3 | 0.70 | 4.46E−03 | 9.15E−03 |
| TTLL4 | 0.70 | 2.05E−03 | 4.95E−03 |
| CCNE2 | 0.70 | 4.67E−03 | 9.47E−03 |
| VRK1 | 0.70 | 3.22E−03 | 7.11E−03 |
| KIAA0101 | 0.70 | 1.13E−04 | 5.83E−04 |
| NT5C2 | 0.70 | 2.09E−05 | 1.93E−04 |
| NECTIN2 | 0.70 | 3.29E−04 | 1.24E−03 |
| PNISR | 0.70 | 1.98E−03 | 4.82E−03 |
| SSRP1 | 0.70 | 4.33E−06 | 7.81E−05 |
| LRR1 | 0.70 | 1.59E−03 | 4.09E−03 |
| CARS | 0.70 | 4.14E−05 | 2.98E−04 |
| LOC105369205 | 0.70 | 2.80E−05 | 2.33E−04 |
| RUVBL2 | 0.70 | 1.04E−05 | 1.30E−04 |
| CPSF4 | 0.70 | 1.82E−03 | 4.51E−03 |
| POLR3K | 0.70 | 1.03E−04 | 5.55E−04 |
| DONSON | 0.70 | 1.96E−03 | 4.78E−03 |
| HADH | 0.70 | 1.07E−04 | 5.65E−04 |
| HSPBP1 | 0.70 | 9.03E−05 | 5.07E−04 |
| HNRNPD | 0.70 | 1.31E−06 | 4.29E−05 |
| ETFA | 0.70 | 8.68E−06 | 1.18E−04 |
| TTC31 | 0.70 | 1.99E−05 | 1.87E−04 |
| CDON | 0.69 | 1.66E−04 | 7.64E−04 |
| NOL4L | 0.69 | 7.36E−04 | 2.25E−03 |
| POLD1 | 0.69 | 8.12E−05 | 4.68E−04 |
| ETV4 | 0.69 | 2.63E−04 | 1.05E−03 |
| ANO6 | 0.69 | 2.65E−06 | 5.98E−05 |
| TYSND1 | 0.69 | 6.48E−04 | 2.06E−03 |
| C7orf50 | 0.69 | 4.94E−05 | 3.33E−04 |
| NR2F6 | 0.69 | 1.59E−04 | 7.41E−04 |
| APOBEC3B | 0.69 | 3.41E−03 | 7.42E−03 |
| ANXA6 | 0.69 | 1.47E−05 | 1.59E−04 |
| MVK | 0.69 | 2.48E−04 | 1.01E−03 |
| JUND | 0.69 | 1.79E−04 | 8.00E−04 |
| KPNB1 | 0.69 | 1.11E−05 | 1.36E−04 |
| LIG3 | 0.69 | 7.35E−05 | 4.38E−04 |
| SNAI1 | 0.69 | 1.07E−04 | 5.67E−04 |
| RAD54L | 0.69 | 8.71E−05 | 4.93E−04 |
| ZNF227 | 0.69 | 1.47E−03 | 3.83E−03 |
| NLGN2 | 0.69 | 4.46E−07 | 2.46E−05 |
| SMC2 | 0.69 | 2.39E−05 | 2.09E−04 |
| NFIX | 0.69 | 1.66E−05 | 1.68E−04 |
| TAF1D | 0.69 | 3.14E−04 | 1.19E−03 |
| CENPW | 0.69 | 1.58E−03 | 4.07E−03 |
| MRPS30 | 0.69 | 2.56E−04 | 1.03E−03 |
| GTF2H3 | 0.69 | 3.92E−05 | 2.89E−04 |
| SHC1 | 0.69 | 7.35E−07 | 3.08E−05 |
| MAGED1 | 0.69 | 4.74E−06 | 8.23E−05 |
| JUN | 0.69 | 6.49E−04 | 2.06E−03 |
| NUP160 | 0.69 | 5.43E−06 | 8.80E−05 |
| TAB3 | 0.69 | 4.97E−04 | 1.69E−03 |
| MELK | 0.69 | 5.94E−04 | 1.92E−03 |
| NSMCE4A | 0.69 | 7.78E−04 | 2.34E−03 |
| CADM1 | 0.69 | 9.99E−05 | 5.43E−04 |
| GINS1 | 0.69 | 3.65E−04 | 1.34E−03 |
| FMNL2 | 0.69 | 8.53E−06 | 1.17E−04 |
| FOXM1 | 0.68 | 6.16E−05 | 3.89E−04 |
| TPCN1 | 0.68 | 7.05E−04 | 2.18E−03 |
| FASTKD3 | 0.68 | 4.69E−03 | 9.51E−03 |
| PARP1 | 0.68 | 4.21E−07 | 2.46E−05 |
| PFAS | 0.68 | 3.73E−05 | 2.79E−04 |
| PIDD1 | 0.68 | 6.40E−04 | 2.04E−03 |
| PCID2 | 0.68 | 1.15E−03 | 3.16E−03 |
| SUFU | 0.68 | 7.28E−04 | 2.23E−03 |
| HSPB1 | 0.68 | 4.39E−05 | 3.09E−04 |
| LMNB2 | 0.68 | 1.19E−05 | 1.42E−04 |
| TSEN2 | 0.68 | 3.75E−03 | 8.00E−03 |
| RAB34 | 0.68 | 1.30E−04 | 6.42E−04 |
| EARS2 | 0.68 | 1.26E−03 | 3.39E−03 |
| TCF12 | 0.68 | 4.31E−05 | 3.05E−04 |
| PRIM1 | 0.68 | 2.58E−03 | 5.94E−03 |
| CBWD5 | 0.68 | 3.50E−04 | 1.30E−03 |
| RTKN | 0.68 | 1.10E−04 | 5.78E−04 |
| MCM8 | 0.68 | 1.13E−03 | 3.13E−03 |
| SRGAP2 | 0.68 | 5.21E−06 | 8.68E−05 |
| DNAJC2 | 0.68 | 3.77E−05 | 2.81E−04 |
| ARHGAP5-AS1 | 0.68 | 2.76E−03 | 6.28E−03 |
| HMGB1 | 0.68 | 2.76E−07 | 2.17E−05 |
| GCLM | 0.68 | 3.31E−04 | 1.24E−03 |
| CCNC | 0.68 | 2.91E−03 | 6.55E−03 |
| WDR90 | 0.68 | 2.06E−03 | 4.97E−03 |
| DPYSL5 | 0.68 | 3.58E−04 | 1.32E−03 |
| RPA3 | 0.68 | 3.20E−03 | 7.08E−03 |
| RRAS2 | 0.68 | 1.17E−04 | 5.99E−04 |
| GPX8 | 0.68 | 2.65E−03 | 6.08E−03 |
| CCDC59 | 0.68 | 1.59E−04 | 7.42E−04 |
| PREP | 0.68 | 3.79E−06 | 7.17E−05 |
| CTU2 | 0.68 | 4.54E−03 | 9.28E−03 |
| HN1 | 0.68 | 9.54E−05 | 5.27E−04 |
| STAG3L2 | 0.68 | 4.75E−03 | 9.60E−03 |
| CSPP1 | 0.68 | 6.98E−04 | 2.17E−03 |
| ANP32E | 0.68 | 1.36E−05 | 1.54E−04 |
| EXOSC7 | 0.68 | 1.03E−03 | 2.91E−03 |
| PKP3 | 0.68 | 2.43E−05 | 2.11E−04 |
| LPIN1 | 0.68 | 1.30E−04 | 6.42E−04 |
| XPO1 | 0.67 | 4.69E−05 | 3.25E−04 |
| EFHD2 | 0.67 | 1.70E−04 | 7.74E−04 |
| NUP37 | 0.67 | 5.05E−04 | 1.71E−03 |
| GALNT2 | 0.67 | 2.14E−06 | 5.42E−05 |
| GORAB | 0.67 | 1.59E−03 | 4.07E−03 |
| FH | 0.67 | 2.06E−05 | 1.91E−04 |
| FAM210A | 0.67 | 3.52E−03 | 7.60E−03 |
| COQ2 | 0.67 | 7.76E−05 | 4.55E−04 |
| VOPP1 | 0.67 | 5.89E−04 | 1.91E−03 |
| LPIN2 | 0.67 | 1.33E−05 | 1.51E−04 |
| SRRT | 0.67 | 6.69E−06 | 9.99E−05 |
| ANK3 | 0.67 | 7.14E−05 | 4.30E−04 |
| GLUD1 | 0.67 | 2.56E−06 | 5.86E−05 |
| COASY | 0.67 | 2.29E−04 | 9.47E−04 |
| ZNF706 | 0.67 | 1.25E−03 | 3.36E−03 |
| SH3TC1 | 0.67 | 9.49E−04 | 2.73E−03 |
| TP53 | 0.67 | 1.60E−04 | 7.43E−04 |
| TIPIN | 0.67 | 3.34E−03 | 7.30E−03 |
| ALDH7A1 | 0.67 | 5.39E−06 | 8.78E−05 |
| CHAC2 | 0.67 | 7.94E−05 | 4.61E−04 |
| PXYLP1 | 0.67 | 6.97E−05 | 4.24E−04 |
| ESPL1 | 0.67 | 3.48E−04 | 1.29E−03 |
| PAK1IP1 | 0.67 | 3.83E−04 | 1.39E−03 |
| ALYREF | 0.67 | 1.24E−04 | 6.26E−04 |
| LOC107987001 | 0.67 | 3.34E−03 | 7.31E−03 |
| HSF2 | 0.67 | 8.35E−04 | 2.46E−03 |
| TSEN15 | 0.67 | 1.04E−03 | 2.93E−03 |
| MCM2 | 0.67 | 6.27E−05 | 3.94E−04 |
| DLEU2L | 0.67 | 1.19E−03 | 3.25E−03 |
| NDC1 | 0.67 | 2.99E−04 | 1.15E−03 |
| SGO2 | 0.67 | 2.74E−04 | 1.08E−03 |
| DNAJC6 | 0.67 | 4.00E−05 | 2.91E−04 |
| PRMT5 | 0.66 | 3.78E−04 | 1.37E−03 |
| CEP55 | 0.66 | 7.42E−05 | 4.41E−04 |
| L1CAM | 0.66 | 2.95E−05 | 2.42E−04 |
| CCDC58 | 0.66 | 1.38E−04 | 6.72E−04 |
| SLC30A1 | 0.66 | 7.96E−05 | 4.62E−04 |
| CCDC88A | 0.66 | 5.73E−05 | 3.71E−04 |
| NDUFAF7 | 0.66 | 1.04E−04 | 5.58E−04 |
| 9-Sep | 0.66 | 3.45E−06 | 6.77E−05 |
| NUP54 | 0.66 | 2.27E−03 | 5.37E−03 |
| PLK4 | 0.66 | 3.17E−03 | 7.03E−03 |
| MRPL57 | 0.66 | 6.98E−04 | 2.17E−03 |
| ANP32B | 0.66 | 5.94E−06 | 9.35E−05 |
| SEPHS1 | 0.66 | 5.25E−04 | 1.76E−03 |
| RFC2 | 0.66 | 7.08E−04 | 2.19E−03 |
| SKA2 | 0.66 | 9.09E−05 | 5.08E−04 |
| TRIM74 | 0.66 | 2.58E−04 | 1.04E−03 |
| LRP6 | 0.66 | 3.93E−06 | 7.37E−05 |
| AARS | 0.66 | 9.65E−06 | 1.27E−04 |
| ECI1 | 0.66 | 1.05E−04 | 5.61E−04 |
| SHTN1 | 0.66 | 8.24E−04 | 2.44E−03 |
| ABI2 | 0.66 | 3.67E−04 | 1.35E−03 |
| NOL12 | 0.66 | 1.89E−03 | 4.65E−03 |
| NEDD4L | 0.66 | 1.13E−04 | 5.82E−04 |
| TACC3 | 0.66 | 5.87E−05 | 3.76E−04 |
| BAGE2 | 0.66 | 6.50E−04 | 2.06E−03 |
| MAGOHB | 0.66 | 2.54E−03 | 5.87E−03 |
| CYB5B | 0.66 | 2.96E−05 | 2.43E−04 |
| HIBADH | 0.66 | 2.32E−05 | 2.06E−04 |
| RHPN2 | 0.66 | 2.70E−04 | 1.07E−03 |
| HSP90AA1 | 0.66 | 4.37E−06 | 7.86E−05 |
| TIMM44 | 0.66 | 6.82E−06 | 1.01E−04 |
| CCNG1 | 0.66 | 1.49E−06 | 4.62E−05 |
| ZNF473 | 0.65 | 3.55E−05 | 2.71E−04 |
| FRMD6 | 0.65 | 6.31E−05 | 3.96E−04 |
| NUF2 | 0.65 | 2.46E−06 | 5.82E−05 |
| ST3GAL2 | 0.65 | 1.71E−05 | 1.72E−04 |
| RIMKLB | 0.65 | 7.01E−04 | 2.18E−03 |
| FBXL5 | 0.65 | 5.04E−05 | 3.38E−04 |
| ALDH3B1 | 0.65 | 2.21E−03 | 5.26E−03 |
| IFITM3 | 0.65 | 5.47E−06 | 8.84E−05 |
| CDC45 | 0.65 | 1.01E−03 | 2.86E−03 |
| EXOSC8 | 0.65 | 5.53E−04 | 1.83E−03 |
| PACSIN3 | 0.65 | 1.28E−06 | 4.28E−05 |
| FAM188B | 0.65 | 2.64E−03 | 6.06E−03 |
| SOCS5 | 0.65 | 1.20E−05 | 1.42E−04 |
| HELLS | 0.65 | 1.10E−04 | 5.78E−04 |
| TOB1 | 0.65 | 6.36E−04 | 2.03E−03 |
| FAM64A | 0.65 | 6.92E−05 | 4.22E−04 |
| TKT | 0.65 | 7.66E−07 | 3.12E−05 |
| CDCA8 | 0.65 | 4.83E−06 | 8.32E−05 |
| AKR1C3 | 0.65 | 8.97E−04 | 2.60E−03 |
| STK11IP | 0.65 | 7.14E−05 | 4.30E−04 |
| SLC6A6 | 0.65 | 2.47E−03 | 5.74E−03 |
| CKS2 | 0.65 | 5.18E−05 | 3.44E−04 |
| DAB2 | 0.65 | 6.52E−04 | 2.06E−03 |
| WDR62 | 0.65 | 4.35E−05 | 3.07E−04 |
| BRCA1 | 0.65 | 2.84E−04 | 1.11E−03 |
| MRC2 | 0.65 | 3.41E−04 | 1.27E−03 |
| GINS4 | 0.65 | 1.07E−04 | 5.66E−04 |
| OIP5 | 0.65 | 1.34E−03 | 3.57E−03 |
| DTWD1 | 0.64 | 2.47E−03 | 5.74E−03 |
| GDF11 | 0.64 | 1.15E−04 | 5.91E−04 |
| MMAB | 0.64 | 7.40E−04 | 2.25E−03 |
| NAT14 | 0.64 | 1.78E−03 | 4.43E−03 |
| ENAH | 0.64 | 1.19E−05 | 1.42E−04 |
| KDELC2 | 0.64 | 1.84E−04 | 8.17E−04 |
| RFC4 | 0.64 | 6.38E−04 | 2.03E−03 |
| EZH2 | 0.64 | 1.93E−04 | 8.45E−04 |
| ROCK2 | 0.64 | 2.98E−06 | 6.27E−05 |
| ZNF286A | 0.64 | 5.47E−04 | 1.81E−03 |
| SLC26A2 | 0.64 | 2.34E−07 | 2.12E−05 |
| PRELID3B | 0.64 | 2.37E−05 | 2.09E−04 |
| PARP2 | 0.64 | 4.52E−04 | 1.58E−03 |
| CIITA | 0.64 | 1.27E−05 | 1.48E−04 |
| KANK1 | 0.64 | 4.25E−05 | 3.02E−04 |
| DEPDC1B | 0.64 | 1.89E−07 | 1.88E−05 |
| WDR4 | 0.64 | 2.67E−05 | 2.26E−04 |
| OSMR | 0.64 | 1.89E−03 | 4.65E−03 |
| TSPAN14 | 0.64 | 6.17E−05 | 3.89E−04 |
| CDCA2 | 0.64 | 2.83E−06 | 6.18E−05 |
| ZNF254 | 0.64 | 1.77E−03 | 4.42E−03 |
| RBBP8 | 0.64 | 5.67E−04 | 1.86E−03 |
| SMG1P2 | 0.64 | 4.05E−03 | 8.49E−03 |
| ECT2 | 0.64 | 2.18E−04 | 9.18E−04 |
| HIST1H4C | 0.64 | 6.89E−04 | 2.15E−03 |
| DHRS11 | 0.64 | 1.66E−04 | 7.63E−04 |
| ARHGEF39 | 0.63 | 4.15E−03 | 8.67E−03 |
| CENPU | 0.63 | 1.39E−04 | 6.75E−04 |
| CDCA3 | 0.63 | 1.83E−04 | 8.13E−04 |
| TYRO3 | 0.63 | 1.27E−05 | 1.48E−04 |
| MFSD12 | 0.63 | 1.58E−05 | 1.66E−04 |
| TMA16 | 0.63 | 1.96E−05 | 1.86E−04 |
| CCNH | 0.63 | 2.11E−04 | 8.95E−04 |
| PRC1 | 0.63 | 4.85E−06 | 8.34E−05 |
| POU2F1 | 0.63 | 2.99E−03 | 6.69E−03 |
| LINC00839 | 0.63 | 3.76E−03 | 8.00E−03 |
| C6orf120 | 0.63 | 1.36E−05 | 1.54E−04 |
| LAPTM4B | 0.63 | 2.61E−06 | 5.95E−05 |
| GNAQ | 0.63 | 1.67E−04 | 7.65E−04 |
| MCM7 | 0.63 | 2.08E−07 | 2.00E−05 |
| EGR3 | 0.63 | 5.99E−04 | 1.93E−03 |
| XAGE1B | 0.63 | 1.65E−05 | 1.68E−04 |
| XAGE1E | 0.63 | 1.65E−05 | 1.68E−04 |
| ZNF326 | 0.63 | 4.75E−04 | 1.64E−03 |
| SLC2A1 | 0.63 | 1.50E−05 | 1.61E−04 |
| NXPE3 | 0.63 | 3.26E−05 | 2.57E−04 |
| ITPK1 | 0.63 | 8.34E−06 | 1.15E−04 |
| ATAD3B | 0.63 | 1.45E−05 | 1.58E−04 |
| OMA1 | 0.63 | 1.80E−03 | 4.47E−03 |
| HSD17B4 | 0.63 | 1.64E−05 | 1.68E−04 |
| CMSS1 | 0.63 | 4.00E−03 | 8.42E−03 |
| PLEKHH3 | 0.63 | 4.36E−04 | 1.53E−03 |
| DYRK2 | 0.63 | 2.08E−04 | 8.89E−04 |
| PTEN | 0.63 | 2.23E−06 | 5.50E−05 |
| MKI67 | 0.63 | 1.46E−05 | 1.58E−04 |
| CSGALNACT1 | 0.63 | 1.38E−05 | 1.55E−04 |
| PARPBP | 0.63 | 2.85E−04 | 1.11E−03 |
| CEP95 | 0.63 | 5.89E−04 | 1.91E−03 |
| PTPN14 | 0.63 | 7.67E−06 | 1.10E−04 |
| PAICS | 0.63 | 9.06E−06 | 1.20E−04 |
| KIF18B | 0.62 | 5.90E−05 | 3.78E−04 |
| ARRB2 | 0.62 | 2.98E−03 | 6.66E−03 |
| CENPK | 0.62 | 4.01E−03 | 8.43E−03 |
| RACGAP1 | 0.62 | 5.23E−06 | 8.68E−05 |
| BCCIP | 0.62 | 6.21E−05 | 3.91E−04 |
| NOTCH1 | 0.62 | 2.56E−06 | 5.86E−05 |
| TMPO | 0.62 | 2.55E−05 | 2.19E−04 |
| MT2A | 0.62 | 3.48E−05 | 2.68E−04 |
| SLFN12 | 0.62 | 2.00E−04 | 8.61E−04 |
| CXXC5 | 0.62 | 6.46E−06 | 9.81E−05 |
| DGKD | 0.62 | 8.46E−05 | 4.83E−04 |
| PHGDH | 0.62 | 2.08E−06 | 5.34E−05 |
| RHBDF1 | 0.62 | 1.43E−05 | 1.57E−04 |
| MMP2 | 0.62 | 3.48E−05 | 2.68E−04 |
| HOXB3 | 0.62 | 1.95E−04 | 8.51E−04 |
| SPAG5 | 0.62 | 2.56E−04 | 1.03E−03 |
| HOXC13 | 0.62 | 7.07E−05 | 4.27E−04 |
| MSH2 | 0.62 | 2.72E−05 | 2.29E−04 |
| NDRG4 | 0.62 | 2.26E−04 | 9.40E−04 |
| KIF23 | 0.62 | 7.67E−05 | 4.51E−04 |
| CNN2 | 0.62 | 3.36E−07 | 2.27E−05 |
| CCNF | 0.62 | 2.92E−05 | 2.41E−04 |
| TENM3 | 0.62 | 5.91E−06 | 9.34E−05 |
| NCAPD2 | 0.62 | 2.32E−08 | 9.89E−06 |
| HOXA5 | 0.62 | 4.70E−04 | 1.63E−03 |
| PER1 | 0.61 | 4.92E−05 | 3.32E−04 |
| SLC7A5 | 0.61 | 1.55E−06 | 4.67E−05 |
| LOC101927809 | 0.61 | 2.65E−03 | 6.07E−03 |
| MRPL39 | 0.61 | 2.01E−04 | 8.65E−04 |
| TRIT1 | 0.61 | 1.20E−04 | 6.07E−04 |
| AURKB | 0.61 | 1.06E−04 | 5.65E−04 |
| ITGA3 | 0.61 | 6.12E−06 | 9.48E−05 |
| ITGB5 | 0.61 | 3.85E−06 | 7.27E−05 |
| MTERF1 | 0.61 | 2.17E−03 | 5.18E−03 |
| ARL13B | 0.61 | 1.42E−05 | 1.57E−04 |
| ORC1 | 0.61 | 3.55E−04 | 1.31E−03 |
| C11orf71 | 0.61 | 5.03E−04 | 1.71E−03 |
| RBMS2 | 0.61 | 1.81E−03 | 4.48E−03 |
| PLEKHO1 | 0.61 | 1.80E−04 | 8.02E−04 |
| FDFT1 | 0.61 | 6.24E−05 | 3.92E−04 |
| GTSE1 | 0.61 | 8.02E−05 | 4.64E−04 |
| POLD3 | 0.61 | 3.19E−03 | 7.07E−03 |
| MIR548XHG | 0.61 | 2.26E−04 | 9.40E−04 |
| THEM6 | 0.61 | 2.60E−04 | 1.04E−03 |
| FASN | 0.61 | 2.79E−06 | 6.13E−05 |
| ARHGEF40 | 0.61 | 2.02E−05 | 1.89E−04 |
| PCDH10 | 0.61 | 8.11E−04 | 2.41E−03 |
| HMGN2 | 0.61 | 3.57E−05 | 2.71E−04 |
| SLC7A1 | 0.61 | 1.53E−06 | 4.67E−05 |
| MAPILC3A | 0.60 | 3.60E−03 | 7.74E−03 |
| RAD51AP1 | 0.60 | 6.28E−04 | 2.01E−03 |
| SNHG4 | 0.60 | 1.78E−03 | 4.42E−03 |
| ARHGAP19 | 0.60 | 5.08E−05 | 3.40E−04 |
| DAP | 0.60 | 9.97E−07 | 3.67E−05 |
| CDC42EP4 | 0.60 | 6.31E−05 | 3.96E−04 |
| SMAD3 | 0.60 | 1.59E−08 | 8.56E−06 |
| TRIM16L | 0.60 | 5.06E−04 | 1.71E−03 |
| CSPG4 | 0.60 | 3.76E−06 | 7.17E−05 |
| TBL1X | 0.60 | 3.20E−06 | 6.54E−05 |
| TEAD4 | 0.60 | 1.54E−04 | 7.26E−04 |
| PSAT1 | 0.60 | 1.23E−05 | 1.46E−04 |
| SCLY | 0.60 | 3.18E−04 | 1.20E−03 |
| SLC3A2 | 0.60 | 1.62E−05 | 1.67E−04 |
| SSBP4 | 0.60 | 2.11E−04 | 8.96E−04 |
| MEX3A | 0.60 | 4.11E−04 | 1.46E−03 |
| PTPRJ | 0.60 | 1.70E−04 | 7.74E−04 |
| PBK | 0.60 | 9.52E−04 | 2.73E−03 |
| FANCD2 | 0.60 | 1.14E−04 | 5.88E−04 |
| GOT1 | 0.59 | 3.03E−05 | 2.46E−04 |
| IFT81 | 0.59 | 2.00E−06 | 5.19E−05 |
| CENPF | 0.59 | 2.10E−06 | 5.36E−05 |
| MBNL2 | 0.59 | 4.16E−04 | 1.48E−03 |
| HMMR | 0.59 | 1.56E−04 | 7.31E−04 |
| B9D1 | 0.59 | 9.32E−05 | 5.17E−04 |
| TGFBR2 | 0.59 | 8.25E−05 | 4.74E−04 |
| ZFP36L2 | 0.59 | 4.30E−05 | 3.04E−04 |
| HMG20B | 0.59 | 3.00E−05 | 2.45E−04 |
| CDCA7L | 0.59 | 1.25E−05 | 1.47E−04 |
| TAF5 | 0.59 | 4.70E−05 | 3.25E−04 |
| PPT2 | 0.59 | 5.68E−04 | 1.86E−03 |
| TSPAN12 | 0.59 | 2.03E−03 | 4.92E−03 |
| TSPYL2 | 0.59 | 8.43E−07 | 3.29E−05 |
| SAV1 | 0.59 | 7.85E−07 | 3.14E−05 |
| SIDT2 | 0.59 | 1.94E−05 | 1.84E−04 |
| UACA | 0.59 | 3.10E−05 | 2.51E−04 |
| BUB1 | 0.59 | 6.40E−05 | 4.00E−04 |
| HPCAL1 | 0.59 | 7.53E−07 | 3.10E−05 |
| CDH19 | 0.59 | 5.76E−04 | 1.88E−03 |
| HPDL | 0.59 | 1.26E−04 | 6.30E−04 |
| MYH10 | 0.59 | 1.75E−06 | 4.84E−05 |
| COL4A2 | 0.59 | 5.44E−07 | 2.66E−05 |
| RNF157 | 0.59 | 2.36E−05 | 2.08E−04 |
| KIF2C | 0.58 | 1.42E−05 | 1.57E−04 |
| TRAF1 | 0.58 | 1.12E−04 | 5.82E−04 |
| CT45A3 | 0.58 | 3.19E−03 | 7.06E−03 |
| IFRD1 | 0.58 | 4.06E−04 | 1.45E−03 |
| SHB | 0.58 | 3.14E−04 | 1.19E−03 |
| C8orf58 | 0.58 | 4.47E−05 | 3.13E−04 |
| KRT8 | 0.58 | 6.79E−05 | 4.17E−04 |
| NLK | 0.58 | 4.49E−05 | 3.14E−04 |
| NDC80 | 0.58 | 2.47E−03 | 5.75E−03 |
| HOXB6 | 0.58 | 3.53E−05 | 2.70E−04 |
| BLMH | 0.58 | 3.22E−05 | 2.55E−04 |
| SNX5 | 0.58 | 3.34E−05 | 2.62E−04 |
| MTSS1L | 0.58 | 1.18E−04 | 5.99E−04 |
| FBXW9 | 0.58 | 2.01E−04 | 8.65E−04 |
| RHOBTB3 | 0.58 | 2.28E−05 | 2.04E−04 |
| CDC25B | 0.58 | 1.12E−06 | 3.95E−05 |
| ZFP42 | 0.58 | 1.36E−03 | 3.61E−03 |
| KIAA0586 | 0.58 | 3.67E−04 | 1.35E−03 |
| RAI1 | 0.58 | 5.30E−06 | 8.71E−05 |
| MIS18BP1 | 0.58 | 2.25E−03 | 5.33E−03 |
| ADARB1 | 0.58 | 2.47E−04 | 1.00E−03 |
| IFT172 | 0.58 | 7.98E−04 | 2.38E−03 |
| OSBPL10 | 0.57 | 7.53E−06 | 1.09E−04 |
| PXMP2 | 0.57 | 6.44E−05 | 4.01E−04 |
| STK40 | 0.57 | 1.33E−05 | 1.51E−04 |
| SKA3 | 0.57 | 8.04E−04 | 2.39E−03 |
| SPDL1 | 0.57 | 1.60E−04 | 7.43E−04 |
| TGIF2 | 0.57 | 1.91E−04 | 8.39E−04 |
| SOD3 | 0.57 | 5.22E−05 | 3.46E−04 |
| PHF19 | 0.57 | 4.14E−04 | 1.47E−03 |
| METTL7B | 0.57 | 9.95E−05 | 5.41E−04 |
| LMNB1 | 0.57 | 8.93E−05 | 5.02E−04 |
| AMIGO2 | 0.57 | 8.23E−06 | 1.14E−04 |
| HKR1 | 0.57 | 5.34E−04 | 1.78E−03 |
| PXDC1 | 0.57 | 9.21E−05 | 5.13E−04 |
| PDK4 | 0.57 | 1.01E−04 | 5.48E−04 |
| NEK2 | 0.57 | 1.14E−06 | 3.99E−05 |
| DHCR7 | 0.57 | 1.79E−04 | 8.00E−04 |
| SPATS2L | 0.57 | 1.03E−05 | 1.30E−04 |
| TPX2 | 0.57 | 2.88E−06 | 6.19E−05 |
| LRIG3 | 0.57 | 5.29E−05 | 3.50E−04 |
| P3H3 | 0.57 | 1.03E−05 | 1.30E−04 |
| NEK6 | 0.57 | 1.08E−04 | 5.69E−04 |
| TK1 | 0.56 | 1.08E−05 | 1.34E−04 |
| SPC24 | 0.56 | 2.20E−06 | 5.45E−05 |
| ECH1 | 0.56 | 3.32E−06 | 6.64E−05 |
| INPP5F | 0.56 | 1.04E−06 | 3.80E−05 |
| BCAT1 | 0.56 | 2.01E−04 | 8.66E−04 |
| AKR1B1 | 0.56 | 1.66E−05 | 1.68E−04 |
| TSPAN13 | 0.56 | 3.32E−05 | 2.61E−04 |
| SLMO2-ATP5E | 0.56 | 2.98E−03 | 6.67E−03 |
| SOGA1 | 0.56 | 1.67E−05 | 1.69E−04 |
| PLK1 | 0.56 | 1.04E−05 | 1.30E−04 |
| SSB | 0.56 | 5.33E−05 | 3.51E−04 |
| PLTP | 0.56 | 3.47E−05 | 2.68E−04 |
| SERTAD4-AS1 | 0.56 | 6.01E−05 | 3.83E−04 |
| GEMIN2 | 0.55 | 1.88E−03 | 4.62E−03 |
| FAM92A1 | 0.55 | 7.69E−04 | 2.32E−03 |
| VEGFC | 0.55 | 6.85E−04 | 2.14E−03 |
| DHCR24 | 0.55 | 5.07E−07 | 2.56E−05 |
| BTBD3 | 0.55 | 4.03E−05 | 2.92E−04 |
| SGCE | 0.55 | 3.01E−03 | 6.72E−03 |
| ADM | 0.55 | 2.37E−03 | 5.56E−03 |
| FARP1 | 0.55 | 7.91E−05 | 4.61E−04 |
| TMPRSS5 | 0.55 | 1.55E−03 | 4.00E−03 |
| KIF11 | 0.55 | 7.29E−07 | 3.07E−05 |
| TWIST1 | 0.55 | 8.79E−04 | 2.56E−03 |
| CCNA2 | 0.55 | 3.45E−05 | 2.67E−04 |
| TPD52L1 | 0.55 | 1.55E−03 | 4.00E−03 |
| TFB1M | 0.55 | 4.29E−04 | 1.51E−03 |
| NES | 0.54 | 1.03E−06 | 3.76E−05 |
| LOC105369647 | 0.54 | 1.44E−04 | 6.90E−04 |
| CENPA | 0.54 | 5.48E−05 | 3.59E−04 |
| GNAS | 0.54 | 7.69E−07 | 3.12E−05 |
| ARHGEF3 | 0.54 | 3.42E−05 | 2.66E−04 |
| MYO1B | 0.54 | 1.28E−05 | 1.48E−04 |
| BTD | 0.54 | 8.68E−04 | 2.53E−03 |
| ZFPM2-AS1 | 0.54 | 1.80E−03 | 4.48E−03 |
| TACC2 | 0.54 | 4.17E−05 | 2.99E−04 |
| ZBTB18 | 0.54 | 5.40E−06 | 8.78E−05 |
| CUTC | 0.54 | 1.69E−04 | 7.73E−04 |
| NUSAP1 | 0.54 | 1.44E−05 | 1.58E−04 |
| RCAN1 | 0.54 | 2.10E−05 | 1.93E−04 |
| CTNNBIP1 | 0.53 | 4.78E−05 | 3.27E−04 |
| SNX10 | 0.53 | 1.13E−04 | 5.84E−04 |
| SSFA2 | 0.53 | 9.17E−06 | 1.21E−04 |
| BAG2 | 0.53 | 1.06E−04 | 5.63E−04 |
| EGR1 | 0.53 | 9.90E−05 | 5.39E−04 |
| TNFRSF19 | 0.53 | 1.65E−05 | 1.68E−04 |
| WWP2 | 0.53 | 1.61E−05 | 1.67E−04 |
| DEPDC1 | 0.53 | 1.10E−04 | 5.77E−04 |
| QKI | 0.53 | 2.87E−04 | 1.12E−03 |
| CCNB2 | 0.53 | 2.50E−05 | 2.15E−04 |
| HSDL2 | 0.53 | 5.89E−06 | 9.32E−05 |
| ACSS2 | 0.53 | 3.90E−05 | 2.88E−04 |
| AKAP2 | 0.52 | 5.78E−05 | 3.73E−04 |
| STAG3L5P-PVRIG2P- | 0.52 | 5.64E−04 | 1.85E−03 |
| PILRB | | | |
| DOCK4 | 0.52 | 5.18E−05 | 3.44E−04 |
| FNBP1L | 0.52 | 1.66E−05 | 1.69E−04 |
| GPC1 | 0.52 | 3.67E−05 | 2.76E−04 |
| VEGFA | 0.52 | 1.28E−04 | 6.37E−04 |
| KIF15 | 0.52 | 1.12E−04 | 5.82E−04 |
| ELMO1 | 0.52 | 8.90E−05 | 5.01E−04 |
| PHC2 | 0.52 | 1.70E−06 | 4.78E−05 |
| TMEM135 | 0.52 | 8.62E−05 | 4.90E−04 |
| PCDH7 | 0.52 | 1.39E−04 | 6.74E−04 |
| TNFRSF21 | 0.52 | 2.73E−06 | 6.08E−05 |
| BMP8B | 0.52 | 2.49E−04 | 1.01E−03 |
| SNHG1 | 0.52 | 4.75E−04 | 1.64E−03 |
| ST5 | 0.51 | 2.19E−04 | 9.20E−04 |
| STX3 | 0.51 | 2.66E−04 | 1.06E−03 |
| COL4A1 | 0.51 | 2.62E−08 | 9.89E−06 |
| PSRC1 | 0.51 | 2.75E−06 | 6.08E−05 |
| PFKFB3 | 0.51 | 2.28E−05 | 2.04E−04 |
| FAM84B | 0.51 | 1.16E−05 | 1.40E−04 |
| TNC | 0.51 | 4.25E−06 | 7.73E−05 |
| CDH2 | 0.51 | 5.80E−06 | 9.21E−05 |
| NECTIN3 | 0.51 | 2.48E−05 | 2.14E−04 |
| DOK1 | 0.50 | 8.82E−05 | 4.98E−04 |
| CDKN3 | 0.50 | 3.14E−03 | 6.97E−03 |
| GAS7 | 0.50 | 5.98E−07 | 2.78E−05 |
| ASB9 | 0.50 | 2.24E−05 | 2.02E−04 |
| SREBF1 | 0.50 | 3.57E−06 | 6.94E−05 |
| ANKRD50 | 0.50 | 7.31E−06 | 1.07E−04 |
| KIF20A | 0.50 | 7.43E−06 | 1.08E−04 |
| FTH1 | 0.50 | 2.47E−07 | 2.12E−05 |
| CDC20 | 0.50 | 4.25E−06 | 7.73E−05 |
| LINC00923 | 0.50 | 5.59E−04 | 1.84E−03 |
| ACACA | 0.49 | 1.06E−07 | 1.51E−05 |
| IGF2BP3 | 0.49 | 5.25E−06 | 8.68E−05 |
| RARA | 0.49 | 1.93E−04 | 8.46E−04 |
| NR1H3 | 0.49 | 4.69E−04 | 1.62E−03 |
| AURKA | 0.49 | 1.17E−05 | 1.41E−04 |
| ETNK2 | 0.49 | 9.46E−06 | 1.25E−04 |
| PTX3 | 0.49 | 8.56E−04 | 2.51E−03 |
| SNHG12 | 0.49 | 4.11E−04 | 1.46E−03 |
| CEP70 | 0.49 | 8.49E−04 | 2.49E−03 |
| ME1 | 0.48 | 1.45E−05 | 1.58E−04 |
| PALLD | 0.48 | 7.73E−05 | 4.54E−04 |
| COL12A1 | 0.48 | 5.43E−07 | 2.66E−05 |
| TPM1 | 0.48 | 3.47E−06 | 6.78E−05 |
| ITGA1 | 0.48 | 1.19E−05 | 1.42E−04 |
| LEF1 | 0.48 | 1.23E−04 | 6.19E−04 |
| WDR35 | 0.48 | 4.29E−05 | 3.04E−04 |
| SLC2A3 | 0.48 | 4.76E−05 | 3.26E−04 |
| RASSF8 | 0.48 | 2.77E−07 | 2.17E−05 |
| CAMK2D | 0.48 | 5.28E−05 | 3.49E−04 |
| SPON2 | 0.48 | 8.68E−05 | 4.92E−04 |
| TNS2 | 0.48 | 8.20E−05 | 4.72E−04 |
| SMAD6 | 0.47 | 1.37E−04 | 6.67E−04 |
| HACL1 | 0.47 | 3.54E−06 | 6.88E−05 |
| TTC8 | 0.47 | 3.15E−03 | 6.99E−03 |
| TGFA | 0.47 | 7.99E−07 | 3.17E−05 |
| DDIT3 | 0.47 | 5.55E−04 | 1.83E−03 |
| FZD2 | 0.47 | 8.75E−05 | 4.95E−04 |
| SYTL5 | 0.47 | 1.21E−05 | 1.43E−04 |
| DNM1 | 0.47 | 5.23E−06 | 8.68E−05 |
| DHRS3 | 0.47 | 1.37E−06 | 4.42E−05 |
| SCD | 0.46 | 3.65E−07 | 2.32E−05 |
| RUNX3 | 0.46 | 7.06E−06 | 1.04E−04 |
| MARCKS | 0.46 | 5.47E−05 | 3.58E−04 |
| LOXL2 | 0.46 | 3.46E−07 | 2.29E−05 |
| HR | 0.46 | 6.49E−06 | 9.84E−05 |
| TNFSF9 | 0.46 | 4.19E−04 | 1.48E−03 |
| SHROOM2 | 0.46 | 1.64E−06 | 4.75E−05 |
| IERSL | 0.46 | 1.42E−05 | 1.57E−04 |
| CDCA7 | 0.45 | 1.54E−04 | 7.26E−04 |
| PDE1C | 0.45 | 1.55E−06 | 4.67E−05 |
| LBR | 0.45 | 1.03E−05 | 1.30E−04 |
| NOX4 | 0.45 | 3.08E−04 | 1.18E−03 |
| ZNF92 | 0.45 | 1.07E−04 | 5.67E−04 |
| LOC101929322 | 0.45 | 5.70E−05 | 3.69E−04 |
| NCOA7 | 0.45 | 8.92E−06 | 1.20E−04 |
| PLA2G16 | 0.44 | 2.94E−05 | 2.42E−04 |
| LPCAT3 | 0.44 | 1.32E−05 | 1.50E−04 |
| PRSS23 | 0.44 | 1.56E−06 | 4.67E−05 |
| CPT1A | 0.44 | 8.10E−05 | 4.68E−04 |
| RAMP1 | 0.44 | 1.49E−05 | 1.60E−04 |
| FTL | 0.44 | 3.12E−07 | 2.21E−05 |
| CLIP2 | 0.44 | 7.00E−07 | 3.00E−05 |
| LY6K | 0.43 | 5.96E−06 | 9.37E−05 |
| CNN3 | 0.43 | 6.52E−07 | 2.89E−05 |
| UQCC1 | 0.43 | 5.64E−05 | 3.67E−04 |
| SPATA6 | 0.43 | 5.05E−04 | 1.71E−03 |
| COL5A2 | 0.42 | 1.70E−06 | 4.79E−05 |
| TLE4 | 0.42 | 3.62E−05 | 2.73E−04 |
| MYOF | 0.42 | 4.13E−07 | 2.43E−05 |
| SYNM | 0.42 | 1.70E−05 | 1.71E−04 |
| GALNT10 | 0.42 | 3.97E−05 | 2.90E−04 |
| ITGA6 | 0.42 | 4.07E−07 | 2.42E−05 |
| ZEB2 | 0.42 | 4.45E−04 | 1.56E−03 |
| COL6A1 | 0.42 | 8.29E−06 | 1.15E−04 |
| NO01 | 0.42 | 2.92E−06 | 6.19E−05 |
| FOSL2 | 0.41 | 2.31E−05 | 2.05E−04 |
| DNAH9 | 0.41 | 1.25E−04 | 6.27E−04 |
| AK4 | 0.41 | 3.41E−05 | 2.66E−04 |
| ERO1B | 0.41 | 2.22E−04 | 9.26E−04 |
| WWTR1 | 0.41 | 1.58E−08 | 8.56E−06 |
| ACAA2 | 0.41 | 8.76E−06 | 1.18E−04 |
| LOC105369782 | 0.41 | 2.43E−03 | 5.67E−03 |
| B3GNT7 | 0.41 | 4.19E−05 | 3.00E−04 |
| TBC1D4 | 0.41 | 3.68E−06 | 7.09E−05 |
| MDK | 0.40 | 3.07E−06 | 6.38E−05 |
| ERBB3 | 0.40 | 1.10E−05 | 1.35E−04 |
| MIR4435-2HG | 0.40 | 8.52E−05 | 4.86E−04 |
| MEIS2 | 0.40 | 5.09E−06 | 8.59E−05 |
| SYNPO | 0.40 | 1.43E−05 | 1.57E−04 |
| MGAT5B | 0.40 | 1.25E−05 | 1.47E−04 |
| ZCCHC24 | 0.40 | 1.68E−06 | 4.78E−05 |
| LINC00152 | 0.39 | 1.57E−04 | 7.34E−04 |
| SEMA3C | 0.39 | 7.49E−07 | 3.10E−05 |
| IMPA2 | 0.39 | 1.03E−04 | 5.55E−04 |
| RHOF | 0.39 | 3.42E−05 | 2.66E−04 |
| CNTRL | 0.39 | 1.12E−06 | 3.95E−05 |
| S100B | 0.39 | 4.17E−05 | 2.99E−04 |
| PYROXD2 | 0.39 | 4.08E−04 | 1.45E−03 |
| TRIM62 | 0.38 | 5.44E−06 | 8.81E−05 |
| USP53 | 0.38 | 1.26E−05 | 1.48E−04 |
| FLRT3 | 0.37 | 3.08E−06 | 6.38E−05 |
| PPP2R3A | 0.37 | 6.72E−06 | 1.00E−04 |
| MICAL3 | 0.36 | 8.02E−07 | 3.17E−05 |
| ANXA1 | 0.36 | 3.27E−08 | 1.05E−05 |
| SOCS3 | 0.36 | 1.40E−05 | 1.56E−04 |
| DDIT4 | 0.36 | 1.57E−07 | 1.73E−05 |
| TLR4 | 0.36 | 1.46E−08 | 8.56E−06 |
| NID1 | 0.36 | 8.81E−07 | 3.39E−05 |
| KIRREL | 0.36 | 7.65E−08 | 1.25E−05 |
| TANC2 | 0.36 | 2.24E−05 | 2.02E−04 |
| FAM129A | 0.36 | 1.19E−06 | 4.10E−05 |
| ARHGAP23 | 0.36 | 5.26E−06 | 8.68E−05 |
| TPT1-AS1 | 0.36 | 1.39E−04 | 6.74E−04 |
| LOC105371703 | 0.35 | 1.36E−03 | 3.60E−03 |
| KIF21B | 0.35 | 3.38E−06 | 6.69E−05 |
| EMP2 | 0.35 | 1.84E−05 | 1.78E−04 |
| NFATC2 | 0.35 | 1.63E−06 | 4.75E−05 |
| EPHB3 | 0.35 | 1.85E−05 | 1.79E−04 |
| HOXB9 | 0.35 | 3.61E−05 | 2.72E−04 |
| SNCA | 0.34 | 1.56E−05 | 1.65E−04 |
| AIM1 | 0.34 | 6.10E−06 | 9.47E−05 |
| TIAM1 | 0.34 | 3.25E−06 | 6.59E−05 |
| LMCD1 | 0.34 | 1.42E−05 | 1.57E−04 |
| TINAGL1 | 0.34 | 1.48E−07 | 1.73E−05 |
| BCAR3 | 0.33 | 7.03E−06 | 1.04E−04 |
| SERPINE2 | 0.33 | 2.17E−06 | 5.42E−05 |
| KIAA1217 | 0.33 | 2.51E−06 | 5.85E−05 |
| PCDHGC3 | 0.33 | 7.93E−06 | 1.13E−04 |
| EXTL1 | 0.33 | 1.82E−06 | 4.92E−05 |
| HIVEP3 | 0.33 | 3.97E−06 | 7.38E−05 |
| IL1RAP | 0.32 | 8.94E−06 | 1.20E−04 |
| AJUBA | 0.32 | 4.35E−08 | 1.17E−05 |
| TGM2 | 0.31 | 1.10E−06 | 3.92E−05 |
| PIR | 0.31 | 1.22E−05 | 1.44E−04 |
| PLEKHA4 | 0.31 | 6.07E−06 | 9.46E−05 |
| C1orf106 | 0.30 | 1.52E−04 | 7.18E−04 |
| POU3F2 | 0.30 | 4.73E−05 | 3.26E−04 |
| EYA1 | 0.30 | 4.43E−06 | 7.94E−05 |
| SDC3 | 0.30 | 1.85E−06 | 4.95E−05 |
| COTL1 | 0.29 | 1.41E−07 | 1.71E−05 |
| MAF | 0.29 | 4.51E−06 | 7.98E−05 |
| FAM46B | 0.29 | 1.27E−05 | 1.48E−04 |
| PDP1 | 0.29 | 5.98E−06 | 9.38E−05 |
| GPNMB | 0.29 | 8.05E−06 | 1.13E−04 |
| CNTNAP3B | 0.29 | 1.10E−06 | 3.92E−05 |
| ADAMTS12 | 0.29 | 1.51E−05 | 1.62E−04 |
| FOXD3 | 0.28 | 3.25E−05 | 2.56E−04 |
| RAB20 | 0.28 | 1.49E−05 | 1.60E−04 |
| TRIB2 | 0.27 | 9.23E−07 | 3.51E−05 |
| PTPRF | 0.26 | 3.36E−07 | 2.27E−05 |
| GDF15 | 0.26 | 1.26E−04 | 6.29E−04 |
| CDC42EP3 | 0.26 | 1.53E−09 | 3.61E−06 |
| TESC | 0.26 | 1.25E−05 | 1.47E−04 |
| DMRT2 | 0.24 | 4.15E−05 | 2.98E−04 |
| PTGES | 0.24 | 4.53E−07 | 2.47E−05 |
| AFAP1 | 0.23 | 2.38E−07 | 2.12E−05 |
| KRT80 | 0.23 | 1.56E−06 | 4.67E−05 |
| S100A3 | 0.23 | 6.46E−04 | 2.05E−03 |
| C10orf90 | 0.22 | 3.25E−05 | 2.56E−04 |
| WNK4 | 0.22 | 9.75E−08 | 1.41E−05 |
| THBS1 | 0.22 | 8.67E−08 | 1.36E−05 |
| LOXL3 | 0.21 | 4.70E−08 | 1.23E−05 |
| LINC00473 | 0.20 | 3.96E−05 | 2.90E−04 |
| KLF9 | 0.20 | 8.42E−07 | 3.29E−05 |
| MEOX2 | 0.19 | 3.30E−06 | 6.64E−05 |
| PLK2 | 0.19 | 1.16E−06 | 4.05E−05 |
| MOXD1 | 0.19 | 7.58E−06 | 1.09E−04 |
| FAM20C | 0.18 | 5.36E−07 | 2.64E−05 |
| LZTS1 | 0.17 | 2.93E−06 | 6.20E−05 |
| ABCC2 | 0.17 | 5.14E−06 | 8.64E−05 |
| SERPINA3 | 0.16 | 4.19E−06 | 7.69E−05 |
| TNS3 | 0.16 | 1.21E−08 | 8.56E−06 |
| ADORA2B | 0.16 | 1.18E−07 | 1.62E−05 |
| CPA4 | 0.14 | 3.22E−05 | 2.55E−04 |
| F2R | 0.13 | 1.54E−07 | 1.73E−05 |
| EPS8 | 0.13 | 4.47E−07 | 2.46E−05 |
| LIMCH1 | 0.13 | 1.28E−06 | 4.28E−05 |
| AGMO | 0.13 | 4.29E−06 | 7.79E−05 |
| ANKRD1 | 0.12 | 1.79E−05 | 1.76E−04 |
| MGAM2 | 0.11 | 1.84E−08 | 8.62E−06 |
| CDKN1C | 0.11 | 2.99E−07 | 2.21E−05 |
| LOXL4 | 0.10 | 6.14E−06 | 9.50E−05 |
| FST | 0.07 | 2.55E−06 | 5.86E−05 |
| LOC102723854 | 0.07 | 2.70E−04 | 1.07E−03 |
| HAPLN1 | 0.05 | 5.87E−09 | 5.95E−06 |
| NGFR | 0.04 | 7.56E−08 | 1.25E−05 |
| SEMA6B | 0.04 | 8.98E−07 | 3.43E−05 |
| NNMT | 0.03 | 6.72E−07 | 2.95E−05 |
| LOC105372338 | 0.03 | 2.13E−05 | 1.95E−04 |

TABLE 7
Co-IP followed by mass spectrometry characterization of B3GNT2 target genes.

| | Alternate | MW | N-term | N-term | C-term | C-term | | Alternate | MW | N-term | N-term | C-term | C-term |
|---|---|---|---|---|---|---|---|---|---|---|---|---|---|
| Accession Number | ID | kDa | IgG | FLAG | IgG | flag | Accession Number | ID | kDa | IgG | FLAG | IgG | flag |
| NP_006568.2 | B3GNT2 | 46 | 0 | 130 | 0 | 132 | NP_001265398.1 (+7) | MON2 | 186 | 0 | 0 | 0 | 3 |
| NP_006077.2 | TUBB3 | 50 | 0 | 115 | 0 | 82 | NP_872590.1 | PCNA | 29 | 0 | 3 | 0 | 0 |
| NP_001060.1 | TUBB2A | 50 | 0 | 128 | 0 | 0 | NP_001180499.1 (+1) | CSRP1 | 20 | 0 | 0 | 0 | 3 |
| NP_001290453.1 | TUBB6 | 50 | 0 | 63 | 0 | 42 | NP_002262.4 (+1) | IPO5 | 124 | 0 | 0 | 0 | 3 |
| NP_003312.3 | TUFM | 50 | 0 | 56 | 0 | 26 | NP_066964.1 | XRCC5 | 83 | 0 | 0 | 0 | 3 |
| NP_006073.2 | TUBA1B | 50 | 0 | 76 | 0 | 0 | NP_006861.1 | DSTN | 19 | 0 | 0 | 0 | 3 |
| NP_003290.1 | HSP90B1 | 92 | 0 | 9 | 0 | 56 | NP_002782.1 | PSMA6 | 27 | 0 | 3 | 0 | 0 |
| NP_733765.1 | ATP2A2 | 115 | 0 | 8 | 0 | 45 | NP_002800.2 | PSMD3 | 61 | 0 | 3 | 0 | 0 |
| NP_005304.3 | PDIA3 | 57 | 0 | 14 | 0 | 24 | NP_006494.1 | PSMC4 | 47 | 0 | 3 | 0 | 0 |
| NP_006614.2 | PHGDH | 57 | 0 | 29 | 0 | 7 | NP_004658.3 (+3) | HERC2 | 527 | 0 | 0 | 0 | 3 |
| NP_006079.1 | TUBB4B | 50 | 0 | 161 | 13 | 119 | NP_001120869.1 (+2) | ERLEC1 | 52 | 0 | 0 | 0 | 3 |
| NP_008835.5 | PRKDC | 469 | 0 | 24 | 0 | 11 | NP_001350685.1 (+3) | ECPAS | 204 | 0 | 3 | 0 | 0 |
| NP_001627.2 | SLC25A6 | 33 | 0 | 15 | 0 | 19 | XP_011541145.1 | ATM | 334 | 0 | 3 | 0 | 0 |
| NP_817092.1 | PPM1G | 59 | 0 | 9 | 0 | 22 | NP_005207.2 | DDOST | 51 | 0 | 0 | 0 | 3 |
| NP_060555.2 | IPO9 | 116 | 0 | 6 | 0 | 24 | NP_001307539.1 (+3) | CHCHD6 | 27 | 0 | 0 | 0 | 3 |
| NP_006380.1 (+1) | HYOU1 | 111 | 0 | 0 | 0 | 27 | NP_001153682.1 (+5) | SLC25A13 | 74 | 0 | 3 | 0 | 0 |
| NP_001269241.1 | FAM3A | 25 | 0 | 9 | 0 | 18 | NP_006088.2 | MYL9 | 20 | 0 | 3 | 0 | 0 |
| NP_004334.1 | CALR | 48 | 0 | 6 | 0 | 20 | NP_002793.2 | PSMC1 | 49 | 0 | 3 | 0 | 0 |
| NP_000909.2 | P4HB | 57 | 0 | 3 | 0 | 23 | NP_001035933.3 (+2) | SUMF2 | 34 | 0 | 0 | 0 | 3 |
| NP_001035109.1 | FAM3C | 25 | 0 | 2 | 0 | 23 | NP_001000.2 | RPS5 | 23 | 3 | 4 | 0 | 6 |
| NP_821133.1 | TUBB | 50 | 10 | 164 | 10 | 117 | NP_005800.3 | PRDX2 | 22 | 5 | 9 | 0 | 5 |
| NP_001186600.1 (+1) | CALU | 38 | 0 | 10 | 0 | 13 | NP_859048.1 | PRDX1 | 22 | 5 | 8 | 0 | 6 |
| NP_000692.2 (+2) | ATP1A1 | 113 | 0 | 11 | 0 | 12 | NP_003236.3 | TGM3 | 77 | 2 | 9 | 3 | 5 |
| NP_001258766.1 (+1) | RCN2 | 39 | 0 | 7 | 0 | 16 | NP_001269091.1 | CAPZB | 34 | 2 | 2 | 0 | 5 |
| NP_001142.2 | SLC25A4 | 33 | 0 | 9 | 0 | 14 | NP_002643.1 | PIP | 17 | 0 | 4 | 2 | 3 |
| NP_057226.1 (+1) | HSD17B12 | 34 | 0 | 10 | 0 | 10 | NP_001009.1 | RPS15 | 17 | 5 | 7 | 2 | 11 |
| NP_078934.3 | IPO4 | 119 | 0 | 3 | 0 | 17 | NP_000977.1 | RPL24 | 18 | 4 | 4 | 3 | 14 |
| NP_006450.2 | ERLIN1 | 39 | 0 | 11 | 0 | 9 | NP_001007.2 | RPS12 | 15 | 0 | 3 | 4 | 8 |
| NP_001269633.1 (+4) | PDIA6 | 54 | 0 | 5 | 0 | 14 | NP_036246.1 | CASP14 | 28 | 3 | 8 | 4 | 9 |
| NP_006827.1 | GCN1 | 293 | 0 | 14 | 0 | 4 | NP_000997.1 | RPS3A | 30 | 9 | 12 | 10 | 29 |
| NP_003891.1 | SQSTM1 | 48 | 0 | 3 | 3 | 43 | NP_001030168.1 | RPL14 | 23 | 7 | 11 | 8 | 21 |
| NP_036555.1 | RPL13A | 24 | 0 | 4 | 0 | 13 | NP_000980.1 | RPL30 | 13 | 6 | 8 | 4 | 14 |
| NP_001349807.1 | ERLIN2 | 38 | 0 | 8 | 0 | 9 | NP_000967.1 | RPL12 | 18 | 4 | 9 | 5 | 11 |
| NP_001350924.1 | CANX | 71 | 0 | 6 | 0 | 11 | NP_001002.1 | RPS7 | 22 | 2 | 8 | 5 | 8 |
| NP_003357.2 | UQCRC2 | 48 | 0 | 13 | 0 | 4 | NP_000975.2 | RPL23A | 18 | 3 | 6 | 4 | 10 |
| NP_006816.2 | CKAP4 | 66 | 0 | 2 | 0 | 14 | NP_001004.2 | RPS9 | 23 | 3 | 2 | 0 | 6 |
| NP_000993.1 | RPLPO | 34 | 0 | 6 | 0 | 10 | NP_006187.2 | PCBP1 | 37 | 0 | 4 | 3 | 4 |
| NP_001530.1 | DNAJA1 | 45 | 0 | 6 | 0 | 10 | NP_006126.1 (+1) | CAPZA1 | 33 | 0 | 2 | 0 | 0 |
| NP_068758.3 (+1) | FKBP10 | 64 | 0 | 0 | 0 | 16 | NP_002323.2 (+1) | LRP1 | 505 | 0 | 2 | 0 | 0 |
| NP_005338.1 | HSPA5 | 72 | 9 | 70 | 12 | 130 | NP_009175.2 | PRR4 | 15 | 0 | 2 | 0 | 0 |
| NP_000971.1 | RPL18A | 21 | 0 | 3 | 0 | 12 | NP_001026854.1 (+1) | SRSF7 | 27 | 0 | 2 | 0 | 0 |
| NP_001273432.1 (+3) | HSPH1 | 92 | 0 | 8 | 0 | 7 | NP_002924.1 | RNASE1 | 18 | 0 | 0 | 0 | 2 |
| NP_001130000.1 (+1) | SERPINE2 | 44 | 0 | 4 | 0 | 11 | XP_016858423.1 | CEP170 | 176 | 0 | 0 | 0 | 2 |
| NP_001888.2 | CSPG4 | 251 | 0 | 2 | 0 | 12 | NP_004516.2 | LRP2 | 522 | 0 | 0 | 0 | 2 |
| NP_005879.1 (+1) | SLC25A3 | 40 | 0 | 4 | 0 | 9 | NP_005900.2 | MAP1B | 271 | 0 | 0 | 0 | 2 |
| NP_001243463.1 | SLC25A1 | 35 | 0 | 5 | 0 | 8 | NP_002777.1 (+1) | PSMA1 | 30 | 0 | 2 | 0 | 0 |
| NP_115587.6 (+1) | LOXL4 | 84 | 0 | 5 | 0 | 8 | NP_002130.2 | RBMX | 42 | 0 | 0 | 0 | 2 |
| NP_001317145.1 (+1) | CNP | 45 | 0 | 5 | 0 | 7 | NP_001292553.1 | CALM2 | 22 | 0 | 0 | 0 | 2 |
| NP_001017958.1 (+3) | OS9 | 74 | 0 | 3 | 0 | 9 | NP_001230106.1 (+1) | ALDOA | 45 | 0 | 2 | 0 | 0 |
| NP_005753.1 (+1) | TRIM28 | 89 | 0 | 4 | 0 | 8 | XP_016873867.1 | RAB1B | 26 | 0 | 0 | 0 | 2 |
| NP_001303963.1 (+3) | DPM1 | 33 | 0 | 4 | 0 | 8 | NP_536350.2 (+3) | GNAS | 111 | 0 | 0 | 0 | 2 |
| NP_001284644.1 (+3) | SMN1 | 30 | 0 | 10 | 2 | 14 | NP_690601.1 | ARPC2 | 34 | 0 | 0 | 0 | 2 |
| NP_573566.2 (+1) | LRPPRC | 158 | 0 | 2 | 0 | 9 | NP_000282.1 | PGK1 | 45 | 0 | 0 | 0 | 2 |
| NP_064508.3 (+1) | TM9SF3 | 68 | 0 | 0 | 0 | 11 | NP_056009.1 (+1) | MYO1D | 116 | 0 | 0 | 0 | 2 |
| NP_005372.2 | NCL | 77 | 0 | 0 | 0 | 11 | NP_001263347.1 (+15) | SEC16A | 249 | 0 | 2 | 0 | 0 |
| NP_001257817.1 (+1) | SLC25A10 | 32 | 0 | 5 | 0 | 6 | NP_001020262.1 (+1) | ABCF1 | 96 | 0 | 0 | 0 | 2 |
| NP_002151.2 (+4) | TNC | 241 | 0 | 0 | 0 | 10 | NP_002780.1 | PSMA4 | 29 | 0 | 2 | 0 | 0 |
| NP_000983.1 (+1) | RPL29 | 18 | 0 | 4 | 0 | 6 | NP_002282.2 (+1) | LAMB1 | 198 | 0 | 0 | 0 | 2 |
| XP_024308303.1 | HLA-A | 48 | 0 | 6 | 0 | 4 | NP_000933.1 | PPIB | 24 | 0 | 0 | 0 | 2 |
| NP_002256.2 | KPNB1 | 97 | 0 | 3 | 0 | 7 | NP_006808.1 | ERP29 | 29 | 0 | 2 | 0 | 0 |
| NP_004199.1 (+1) | AIFM1 | 67 | 0 | 5 | 0 | 5 | NP_112233.2 | SFXN3 | 36 | 0 | 0 | 0 | 2 |
| NP_001341088.1 | ACSL3 | 80 | 0 | 5 | 0 | 5 | NP_001177990.1 | SLC25A22 | 34 | 0 | 0 | 0 | 2 |
| NP_001290043.1 (+2) | TUBA1C | 58 | 8 | 76 | 14 | 63 | NP_001191791.1 | ELOC | 12 | 0 | 0 | 0 | 2 |
| NP_001240312.1 | RPL15 | 24 | 0 | 6 | 2 | 15 | NP_001316381.1 (+2) | NTPCR | 21 | 0 | 0 | 0 | 2 |
| NP_001258901.1 (+1) | HSP90AB1 | 82 | 0 | 10 | 2 | 11 | NP_001306165.1 | RPL34 | 13 | 0 | 0 | 0 | 2 |
| NP_001001.2 | RPS6 | 29 | 2 | 11 | 2 | 20 | NP_001128582.1 (+1) | DNAJA3 | 50 | 0 | 0 | 0 | 2 |
| NP_612510.1 (+2) | TECR | 36 | 0 | 0 | 0 | 9 | NP_002781.2 | PSMA5 | 26 | 0 | 2 | 0 | 0 |
| NP_001164014.1 (+1) | PGAM5 | 32 | 0 | 4 | 0 | 5 | NP_001339250.1 (+6) | LARP4 | 63 | 0 | 0 | 0 | 2 |
| NP_001158886.1 (+1) | LDHA | 40 | 0 | 5 | 0 | 4 | NP_002794.1 | PSMC2 | 49 | 0 | 2 | 0 | 0 |
| NP_002892.1 | RCN1 | 39 | 0 | 0 | 0 | 9 | NP_001193496.1 (+3) | IFI16 | 83 | 0 | 0 | 0 | 2 |
| NP_001164006.1 (+2) | ATAD3A | 66 | 0 | 0 | 0 | 9 | NP_001001520.1 (+4) | HDGFL2 | 74 | 0 | 0 | 0 | 2 |
| NP_060908.1 | FBXO6 | 34 | 0 | 4 | 0 | 5 | NP_001070151.1 (+3) | UBTF | 85 | 0 | 0 | 0 | 2 |
| NP_006382.1 | IPO7 | 120 | 0 | 0 | 0 | 9 | NP_006017.1 | H1FX | 22 | 0 | 0 | 0 | 2 |
| XP_006716248.1 | PDIA4 | 73 | 0 | 0 | 0 | 9 | NP_002787.2 | PSMB4 | 29 | 0 | 2 | 0 | 0 |
| NP_001352542.1 | RRBP1 | 152 | 0 | 2 | 2 | 16 | XP_006719595.1 (+5) | PXN | 121 | 0 | 2 | 0 | 0 |
| NP_001952.1 | EEF2 | 95 | 0 | 3 | 0 | 5 | NP_001072989.1 (+5) | KTN1 | 156 | 0 | 0 | 0 | 2 |
| NP_005446.2 | ZRANB2 | 36 | 0 | 5 | 0 | 3 | NP_055866.1 | ERP44 | 47 | 0 | 0 | 0 | 2 |
| NP_002789.1 | PSMB6 | 25 | 0 | 5 | 0 | 3 | NP_006691.1 | TRAFD1 | 65 | 0 | 0 | 0 | 2 |
| NP_001309856.1 (+1) | STAU1 | 56 | 0 | 2 | 0 | 6 | NP_000024.2 | ABCD1 | 83 | 0 | 0 | 0 | 2 |
| NP_938148.1 (+1) | GANAB | 107 | 0 | 0 | 0 | 8 | NP_001157852.1 (+4) | STAU2 | 63 | 0 | 0 | 0 | 2 |
| NP_001021.1 | RPS27 | 9 | 0 | 2 | 0 | 6 | NP_055634.3 | PJA2 | 78 | 0 | 0 | 0 | 2 |
| NP_001407.1 | EIF4A1 | 46 | 0 | 2 | 0 | 6 | NP_001243028.1 (+1) | CTHRC1 | 25 | 0 | 0 | 0 | 2 |
| XP_005268045.1 | SEL1L | 87 | 0 | 3 | 0 | 5 | NP_001138586.1 (+4) | RRP12 | 137 | 0 | 0 | 0 | 2 |
| NP_001651.1 | ARF4 | 21 | 0 | 4 | 0 | 4 | NP_000173.2 | HADHA | 83 | 0 | 0 | 0 | 2 |
| NP_001341935.1 (+1) | NPM1 | 33 | 0 | 0 | 0 | 8 | NP_001185771.1 (+1) | CCT2 | 53 | 0 | 0 | 0 | 2 |
| NP_001143.2 | SLC25A5 | 33 | 6 | 19 | 3 | 30 | NP_004981.2 | MARS | 101 | 0 | 2 | 0 | 0 |
| NP_002147.2 | HSPD1 | 61 | 0 | 10 | 2 | 6 | NP_001243.1 | CD70 | 21 | 0 | 0 | 0 | 2 |
| NP_002435.1 (+2) | MSN | 68 | 0 | 3 | 0 | 4 | NP_055467.3 | IPO13 | 108 | 0 | 0 | 0 | 2 |
| NP_001074419.1 | MYO1C | 120 | 0 | 4 | 0 | 3 | NP_001159757.1 (+1) | CCT7 | 55 | 0 | 0 | 0 | 2 |
| NP_001007074.1 | RPL32 | 16 | 0 | 0 | 0 | 7 | XP_024306583.1 (+1) | ARHGEF2 | 104 | 0 | 0 | 0 | 2 |
| NP_001186757.1 | SPRR1A | 10 | 0 | 4 | 0 | 3 | NP_115497.4 | MAGT1 | 42 | 0 | 0 | 0 | 2 |
| NP_036350.2 (+2) | ILF3 | 95 | 0 | 2 | 0 | 5 | NP_005085.2 | SLC27A4 | 72 | 0 | 0 | 0 | 2 |
| NP_001350420.1 | ZC3HAV1 | 114 | 0 | 3 | 0 | 4 | NP_001278795.1 (+1) | GALNT2 | 61 | 0 | 0 | 0 | 2 |
| NP_001020092.1 (+1) | RPL9 | 22 | 0 | 0 | 0 | 7 | NP_001336864.1 (+1) | TOR1AIP2 | 51 | 0 | 0 | 0 | 2 |
| NP_057260.2 (+2) | SDF4 | 42 | 0 | 0 | 0 | 7 | NP_001317621.1 (+1) | PSMD4 | 41 | 0 | 2 | 0 | 0 |
| NP_002635.2 | PIGR | 83 | 0 | 2 | 0 | 5 | NP_002878.2 | RARS | 75 | 0 | 0 | 0 | 2 |
| NP_004437.2 | EPRS | 171 | 0 | 3 | 0 | 4 | NP_000978.1 | RPL26 | 17 | 5 | 7 | 4 | 12 |
| NP_064505.1 (+3) | UGGT1 | 177 | 0 | 2 | 0 | 5 | NP_001344872.1 (+1) | GAPDH | 34 | 11 | 18 | 6 | 16 |
| NP_057390.1 | DNAJB11 | 41 | 0 | 3 | 0 | 4 | NP_000998.1 | RPS4X | 30 | 12 | 14 | 7 | 23 |
| NP_002941.1 | RPN1 | 69 | 0 | 0 | 0 | 7 | XP_016882687.1 | UBA52 | 22 | 5 | 4 | 7 | 20 |
| NP_009109.3 | STRAP | 38 | 0 | 4 | 0 | 3 | NP_000230.1 | LYZ | 17 | 4 | 3 | 0 | 6 |
| NP_003135.2 | SSR1 | 32 | 0 | 3 | 0 | 4 | NP_001025180.1 | RPS15A | 15 | 2 | 3 | 2 | 6 |
| XP_016875611.1 (+1) | YBX3 | 40 | 0 | 0 | 0 | 7 | NP_001419.1 | ENO1 | 47 | 2 | 5 | 2 | 4 |
| NP_000995.1 | RPLP2 | 12 | 3 | 6 | 0 | 13 | NP_002955.2 | S100A8 | 11 | 4 | 15 | 5 | 3 |
| NP_002956.1 | S100A9 | 13 | 0 | 18 | 5 | 9 | NP_001019380.2 | SPRR2E | 8 | 3 | 16 | 5 | 0 |
| NP_009035.3 | RPL10A | 25 | 4 | 6 | 0 | 16 | NP_112420.1 | HNRNPA1 | 39 | 4 | 7 | 4 | 9 |
| NP_001435.1 | FABP5 | 15 | 2 | 6 | 0 | 8 | NP_000549.1 | HBA1 | 15 | 3 | 3 | 0 | 4 |
| NP_066357.3 | RPL36A | 12 | 2 | 3 | 0 | 11 | NP_001900.1 | CTSD | 45 | 3 | 3 | 0 | 4 |
| NP_001254792.1 (+1) | SLIRP | 12 | 0 | 0 | 0 | 6 | NP_001091676.1 | HNRNPF | 46 | 0 | 0 | 3 | 7 |
| NP_000984.1 (+2) | RPL31 | 14 | 0 | 0 | 0 | 6 | NP_000973.2 | RPL21 | 19 | 5 | 8 | 5 | 11 |
| NP_066953.1 | PPIA | 18 | 0 | 6 | 0 | 0 | NP_001186731.1 | RPL11 | 20 | 3 | 5 | 3 | 7 |
| NP_001294857.1 (+3) | SERPINB12 | 48 | 0 | 4 | 0 | 2 | NP_001611.1 | AHNAK | 629 | 0 | 3 | 2 | 2 |
| NP_663723.1 | YWHAZ | 28 | 0 | 2 | 0 | 4 | NP_000356.1 (+2) | TPI1 | 27 | 2 | 3 | 0 | 2 |
| NP_000987.2 | RPL35A | 13 | 0 | 0 | 0 | 6 | NP_001006.1 | RPS11 | 18 | 8 | 13 | 9 | 18 |
| NP_378669.1 | RPL36 | 12 | 0 | 0 | 0 | 6 | NP_001393.1 | EEF1A1 | 50 | 8 | 13 | 5 | 11 |
| NP_001238978.1 (+3) | PCMT1 | 30 | 0 | 4 | 0 | 2 | NP_009176.2 | ATXN2L | 113 | 3 | 5 | 2 | 5 |
| NP_003132.2 | TRIM21 | 54 | 0 | 6 | 0 | 0 | NP_001264069.1 (+1) | ACTR3 | 42 | 3 | 3 | 2 | 7 |
| NP_001677.2 | ATP5F1B | 57 | 0 | 2 | 0 | 4 | NP_001012.1 | RPS17 | 16 | 3 | 5 | 5 | 10 |
| NP_061185.1 | RCC2 | 56 | 0 | 0 | 0 | 6 | NP_001308051.1 | LTF | 77 | 6 | 8 | 2 | 7 |
| NP_009135.4 | DDX20 | 92 | 0 | 0 | 0 | 6 | NP_001308412.1 | RPS19 | 16 | 5 | 5 | 3 | 10 |
| NP_001291972.1 | TMEM59 | 36 | 0 | 6 | 0 | 0 | NP_001003.1 | RPS8 | 24 | 7 | 6 | 6 | 17 |
| NP_002783.1 | PSMA7 | 28 | 0 | 4 | 0 | 2 | NP_003008.1 | SRSF3 | 19 | 4 | 6 | 3 | 7 |
| NP_005871.1 | DNAJA2 | 46 | 0 | 3 | 0 | 3 | NP_000989.1 | RPL37A | 10 | 4 | 6 | 3 | 7 |
| XP_011518263.1 | MTCH2 | 30 | 0 | 4 | 0 | 2 | NP_001001414.1 | NCCRP1 | 31 | 4 | 4 | 0 | 4 |
| NP_003137.1 (+3) | SSRP1 | 81 | 0 | 0 | 0 | 6 | NP_001278086.1 | EIF4G1 | 171 | 2 | 2 | 2 | 6 |
| NP_000960.2 | RPL5 | 34 | 0 | 0 | 0 | 6 | NP_002943.2 | RPS2 | 31 | 10 | 13 | 12 | 24 |
| NP_000203.2 | ITGB3 | 87 | 0 | 3 | 0 | 3 | NP_005498.1 | CFL1 | 19 | 3 | 5 | 3 | 6 |
| NP_001350066.1 (+2) | MTDH | 67 | 0 | 0 | 0 | 6 | NP_000036.2 (+1) | ARG1 | 35 | 4 | 8 | 4 | 6 |
| NP_001304663.1 (+2) | PBXIP1 | 78 | 0 | 2 | 0 | 4 | NP_001107018.1 (+1) | LIMA1 | 85 | 3 | 4 | 0 | 2 |
| NP_000981.1 | RPL27A | 17 | 3 | 7 | 0 | 10 | NP_003320.2 | TXN | 12 | 3 | 3 | 0 | 3 |
| NP_000958.1 | RPL3 | 46 | 3 | 8 | 5 | 25 | NP_072045.1 | RPS18 | 18 | 11 | 15 | 9 | 18 |
| NP_000972.1 (+1) | RPL19 | 23 | 2 | 8 | 2 | 11 | NP_001016.1 | RPS23 | 16 | 6 | 9 | 9 | 16 |
| NP_114032.2 | HNRNPU | 91 | 2 | 0 | 0 | 12 | NP_001011.1 | RPS16 | 16 | 10 | 9 | 8 | 20 |
| NP_009140.1 | RPL35 | 15 | 2 | 7 | 0 | 5 | NP_005208.1 (+1) | DEFA3 | 10 | 3 | 5 | 5 | 8 |
| NP_005312.1 | HIST1H1E | 22 | 2 | 6 | 0 | 6 | NP_008855.1 | SRSF1 | 28 | 5 | 4 | 3 | 9 |
| NP_001255.4 | CDSN | 52 | 0 | 5 | 0 | 0 | NP_203754.2 | TNKS1BP1 | 182 | 4 | 2 | 2 | 8 |
| NP_000974.1 | RPL22 | 15 | 0 | 0 | 0 | 5 | NP_001531.1 | HSPB1 | 23 | 4 | 3 | 0 | 4 |
| NP_001001522.1 | TAGLN | 23 | 0 | 2 | 0 | 3 | NP_001008.1 | RPS13 | 17 | 2 | 3 | 2 | 4 |
| NP_001294869.1 (+2) | POF1B | 69 | 0 | 5 | 0 | 0 | NP_000108.1 | EMD | 29 | 0 | 2 | 2 | 2 |
| NP_061865.1 | KCTD5 | 26 | 0 | 2 | 0 | 3 | NP_005608.1 | RPS14 | 16 | 9 | 11 | 10 | 18 |
| NP_000168.1 (+9) | GSN | 86 | 0 | 2 | 0 | 3 | NP_000990.1 | RPL38 | 8 | 5 | 9 | 6 | 8 |
| NP_000631.1 | IL13RA2 | 44 | 0 | 3 | 0 | 2 | NP_001093639.1 (+3) | IMMT | 84 | 17 | 23 | 16 | 26 |
| NP_001348.2 | DHX9 | 141 | 0 | 0 | 0 | 5 | NP_705935.1 | TPM3 | 29 | 5 | 6 | 2 | 5 |
| XP_011532005.1 (+6) | MAP4 | 248 | 0 | 0 | 0 | 5 | NP_001035972.1 | LGALS7B | 15 | 4 | 7 | 3 | 4 |
| NP_056536.2 (+2) | GEMIN4 | 120 | 0 | 0 | 0 | 5 | NP_004930.1 | DDX1 | 82 | 2 | 7 | 5 | 4 |
| NP_008977.1 | EMILIN1 | 107 | 0 | 2 | 0 | 3 | NP_001304106.1 (+1) | CHCHD3 | 27 | 10 | 16 | 11 | 15 |
| NP_001185845.1 (+1) | PPFIBP1 | 110 | 0 | 2 | 0 | 3 | NP_002007.1 | FLG | 435 | 20 | 29 | 15 | 22 |
| NP_057195.2 (+1) | TELO2 | 92 | 0 | 2 | 0 | 3 | NP_002954.2 | S100A7 | 11 | 2 | 3 | 3 | 5 |
| NP_036585.1 | TBL2 | 50 | 0 | 0 | 0 | 5 | NP_001153148.1 (+1) | SYNCRIP | 63 | 6 | 2 | 4 | 13 |
| NP_005989.3 | CCT3 | 61 | 0 | 0 | 0 | 5 | XP_011516977.1 | LCN1 | 32 | 9 | 11 | 6 | 11 |
| NP_006089.1 | RACK1 | 35 | 0 | 2 | 0 | 3 | NP_003741.1 | EIF3A | 167 | 3 | 2 | 0 | 3 |
| NP_001339204.1 (+4) | RHOT2 | 68 | 0 | 3 | 0 | 2 | NP_057342.2 | YTHDF2 | 62 | 4 | 5 | 2 | 4 |
| NP_001186307.1 (+13) | GBF1 | 206 | 0 | 0 | 0 | 5 | NP_001170884.1 | RPS27A | 18 | 11 | 8 | 14 | 27 |
| NP_001304711.1 | RPL8 | 28 | 3 | 10 | 3 | 15 | NP_000969.1 | RPL23 | 15 | 5 | 7 | 4 | 6 |
| NP_000962.2 | RPL7 | 29 | 5 | 10 | 3 | 21 | NP_005959.2 (+1) | HNRNPM | 78 | 5 | 6 | 5 | 8 |
| NP_001350626.1 | RPL28 | 19 | 3 | 8 | 3 | 16 | NP_663780.2 | SYNM | 173 | 5 | 5 | 2 | 5 |
| NP_001020.2 | RPS26 | 13 | 4 | 6 | 0 | 11 | NP_001193725.1 (+3) | PKM | 66 | 2 | 3 | 2 | 3 |
| NP_008850.1 | SERPINB3 | 45 | 3 | 10 | 2 | 9 | XP_016864497.1 | SYNPO | 122 | 4 | 4 | 4 | 7 |
| NP_059118.2 | CALML5 | 16 | 0 | 0 | 0 | 4 | NP_004550.2 | YBX1 | 36 | 3 | 2 | 5 | 9 |
| NP_001288155.1 | TPM2 | 33 | 0 | 0 | 0 | 4 | NP_00159506.1 (+2) | SBSN | 61 | 25 | 27 | 15 | 25 |
| NP_000509.1 | HBB | 16 | 0 | 4 | 0 | 0 | NP_000350.1 | TGM1 | 90 | 9 | 17 | 10 | 8 |
| NP_004986.1 | MMP14 | 66 | 0 | 2 | 0 | 2 | NP_001317259.1 (+2) | LUC7L3 | 58 | 2 | 4 | 3 | 3 |
| NP_002779.1 (+1) | PSMA3 | 28 | 0 | 4 | 0 | 0 | NP_001135757.1 | RPS24 | 32 | 4 | 0 | 2 | 8 |
| NP_002284.3 | LAMC1 | 178 | 0 | 0 | 0 | 4 | NP_001186273.1 (+1) | RPL17 | 21 | 0 | 3 | 2 | 0 |
| NP_001296372.1 (+1) | SPARC | 35 | 0 | 0 | 0 | 4 | NP_005850.1 | PURA | 35 | 2 | 0 | 0 | 3 |
| NP_002788.1 | PSMB5 | 28 | 0 | 4 | 0 | 0 | P0AA25.2 | NA | 12 | 3 | 7 | 8 | 7 |
| NP_001294853.1 (+1) | NAP1L1 | 45 | 0 | 0 | 0 | 4 | NP_055987.2 (+10) | PRRC2C | 309 | 11 | 6 | 6 | 15 |
| NP_001116307.2 (+5) | TTC13 | 91 | 0 | 0 | 0 | 4 | NP_001129512.1 (+5) | CPSF7 | 52 | 8 | 12 | 9 | 9 |
| NP_000293.2 (+1) | PLOD1 | 84 | 0 | 0 | 0 | 4 | P01837.1 | NA | 12 | 87 | 89 | 108 | 146 |
| NP_006657.1 | RUVBL2 | 51 | 0 | 0 | 0 | 4 | NP_006266.2 | SRSF6 | 40 | 3 | 6 | 5 | 4 |
| NP_009166.2 | XPOT | 110 | 0 | 2 | 0 | 2 | NP_001339702.1 | JUP | 82 | 39 | 47 | 36 | 43 |
| NP_001276332.1 (+9) | MMS19 | 108 | 0 | 4 | 0 | 0 | NP_001180345.1 (+1) | DDX3X | 73 | 19 | 18 | 15 | 23 |
| NP_001352606.1 (+3) | P4HA2 | 61 | 0 | 0 | 0 | 4 | NP_001014.1 (+1) | RPS20 | 13 | 4 | 4 | 5 | 7 |
| NP_001027017.1 (+3) | CPT1A | 86 | 0 | 2 | 0 | 2 | NP_001091974.1 | DDX17 | 80 | 23 | 23 | 22 | 30 |
| NP_004362.2 | COPA | 138 | 0 | 2 | 0 | 2 | NP_001351158.1 (+2) | HNRNPH1 | 51 | 11 | 11 | 11 | 15 |
| NP_003391.1 (+3) | XPO1 | 123 | 0 | 2 | 0 | 2 | NP_016868724.1 | PUF60 | 63 | 5 | 6 | 5 | 6 |
| NP_001014842.1 (+1) | TM9SF1 | 55 | 0 | 0 | 0 | 4 | NP_005711.1 | ARPC1B | 41 | 2 | 0 | 2 | 5 |
| NP_000963.1 | RPL7A | 30 | 4 | 8 | 5 | 22 | XP_006319.1 | RBM14 | 69 | 4 | 3 | 0 | 2 |
| NP_001307067.1 | RPL6 | 33 | 5 | 10 | 4 | 20 | NP_001275582.1 (+2) | CLTC | 192 | 6 | 6 | 5 | 7 |
| NP_001019850.1 | C1orf68 | 26 | 0 | 13 | 4 | 2 | NP_001305115.1 (+1) | HNRNPK | 49 | 8 | 9 | 10 | 12 |
| NP_000959.2 | RPL4 | 48 | 11 | 15 | 6 | 36 | NP_00546.2 | KRT6B | 60 | 316 | 370 | 295 | 331 |
| NP_006588.1 | HSPA8 | 71 | 16 | 41 | 15 | 49 | NP_001743.1 | CAT | 60 | 2 | 6 | 3 | 0 |
| NP_002263.3 | KRT4 | 56 | 21 | 22 | 0 | 37 | NP_002128.1 (+1) | HNRNPA2B1 | 36 | 4 | 7 | 9 | 8 |
| NP_000970.1 | RPL18 | 22 | 5 | 11 | 7 | 24 | NP_002810.1 (+4) | PRBP1 | 60 | 7 | 6 | 6 | 9 |
| NP_001290554.1 | RPL10 | 25 | 3 | 4 | 3 | 15 | NP_733821.1 | LMNA | 74 | 11 | 11 | 18 | 22 |
| NP_000968.2 | RPL13 | 24 | 6 | 15 | 8 | 23 | NP_004629.3 (+1) | PRRC2A | 229 | 3 | 2 | 3 | 5 |
| NP_689557.1 | LSM12 | 22 | 2 | 4 | 0 | 4 | NP_002964.3 | ATXN2 | 140 | 3 | 4 | 3 | 3 |
| NP_006251.1 (+4) | DNAJC3 | 58 | 0 | 0 | 0 | 3 | NP_001273001.1 (+1) | MOV10 | 107 | 3 | 4 | 3 | 3 |
| NP_001123910.1 (+10) | SPTAN1 | 285 | 0 | 0 | 0 | 3 | NP_003925.1 (+2) | FUBP3 | 62 | 8 | 8 | 7 | 9 |
| NP_076956.1 | GGCT | 21 | 0 | 3 | 0 | 0 | NP_001307526.1 | DDX5 | 69 | 18 | 16 | 16 | 22 |
| NP_056195.3 | SAMM50 | 52 | 0 | 3 | 0 | 0 | NP_065823.1 | NUFIP2 | 76 | 3 | 3 | 4 | 5 |
| NP_056988.3 | EIF5B | 139 | 0 | 0 | 0 | 3 | NP_055662.3 (+3) | UBAP2L | 115 | 20 | 20 | 15 | 19 |
| NP_00108494.1 (+1) | PUM1 | 127 | 0 | 0 | 0 | 3 | NP_005745.1 | G3BP1 | 52 | 29 | 27 | 25 | 33 |
| NP_001022.1 | RPS28 | 8 | 0 | 0 | 0 | 3 | NP_001310351.1 (+3) | RBM39 | 56 | 5 | 4 | 3 | 5 |
| NP_001243064.1 (+2) | CSE1L | 104 | 0 | 0 | 0 | 3 | NP_001035202.1 (+4) | MYH11 | 224 | 3 | 2 | 5 | 7 |
| NP_000174.1 (+3) | HADHB | 51 | 0 | 0 | 0 | 3 | NP_001243731.1 (+1) | RPS3 | 27 | 13 | 10 | 17 | 23 |
| NP_001609.2 | PARP1 | 113 | 0 | 0 | 0 | 3 | NP_001018077.1 (+1) | SERBP1 | 45 | 4 | 0 | 5 | 10 |
| NP_001185709.1 (+1) | ARPC4 | 22 | 0 | 0 | 0 | 3 | NP_001186883.1 | ACTG1 | 42 | 103 | 82 | 71 | 107 |
| NP_113584.3 (+11) | HUWE1 | 482 | 0 | 3 | 0 | 0 | NP_001988.1 | FAU | 14 | 6 | 5 | 5 | 7 |
| NP_009123.1 (+1) | SUPT16H | 120 | 0 | 0 | 0 | 3 | NP_002559.2 | PABPC1 | 71 | 37 | 31 | 30 | 41 |
| NP_003192.1 | TFAM | 29 | 0 | 0 | 0 | 3 | NP_005889.3 (+1) | CAPRIN1 | 78 | 6 | 7 | 6 | 6 |
| NP_003748.1 | EIF3I | 37 | 0 | 0 | 0 | 3 | NP_001287876.1 (+3) | CPSF6 | 63 | 28 | 30 | 33 | 35 |
| NP_803182.1 | MAGED2 | 65 | 0 | 0 | 0 | 3 | NP_003478.1 (+1) | TAF15 | 62 | 10 | 5 | 4 | 10 |
| NP_003125.3 | SRP14 | 15 | 0 | 0 | 0 | 3 | NP_006550.1 | KHDRBS1 | 48 | 10 | 9 | 7 | 9 |
| NP_031385.2 | CASC3 | 76 | 0 | 3 | 0 | 0 | NP_001604.1 (+2) | ACTA2 | 42 | 77 | 64 | 63 | 81 |
| NP_000353.1 | TIMP3 | 24 | 0 | 0 | 0 | 3 | NP_003371.2 | VIM | 54 | 452 | 402 | 337 | 411 |
| NP_110379.2 | TCP1 | 60 | 0 | 0 | 0 | 3 | NP_001002858.1 (+1) | ANXA2 | 40 | 15 | 17 | 16 | 15 |

### Methods

### Sequences and Cloning

The plasmids lenti dCAS-VP64_Blast (Addgene 61425), lenti sgRNA(MS2)_zeo backbone (Addgene 61427), and lentiMPHv2 (Addgene 89308) were used for CRISPR-Cas9 activation. The human SAM CRISPR activation library (Addgene 1000000057) was used for CRISPR-Cas9 activation screening. LentiCRISPRv2 (Addgene 52961) was used for CRISPR-Cas9 knockout. The Cas9 in lentiCRISPRv2 was replaced with dCas9-KRAB (Addgene 46911) and the Puromycin resistance gene was replaced with Blasticidin resistance gene (Addgene 75112) for CRISPR-Cas9 knockdown. Single guide RNA (sgRNA) spacer sequences used in this study are listed in Table 8, and were cloned into the respective vectors as previously described (Joung et al., 2017). The NY-ESO-1 T cell receptor (TCR) clone 1G4 (Robbins et al., 2008), AXL chimeric antigen receptor (CAR) (Cho et al., 2018), and HER2 CAR (Cho et al., 2018) were synthesized and cloned into the pHR TCR vector (Addgene 89347). The respective ORFs of candidate genes [CD274 (NM_014143), MCL1 (NM_021960), JUNB (NM_002229), and B3GNT2 (NM_006577)] were synthesized and cloned into the plasmid pLX_TRC209 (Broad Genetic Perturbation Platform) for overexpression. HLA-A2 (Addgene 85162), ESO:HLA-A2, and Gaussia luciferase were cloned into pLX_TRC209 for stable expression. For dox-inducible upregulation, the EF1a promoter in pLX_TRC209 was replaced with the pTight promoter (Addgene 31877) and the plasmid pUltra-puro-RTTA3 (Addgene 58750) was used for rtTA.

TABLE 8
List of sgRNAs used in the study.
sgRNA ID, perturbation, target
sequences, and target genes.

| Target | sgRNA | | Target sequence |
|---|---|---|---|
| gene | ID | Perturbation | (5′ to 3′) |
| CTAG1A/B | sg1 | knockout | GCGGGGTCCGCATGGCGGCG |
| | | | (SEQ ID NO: 20) |
| CTAG1A/B | sg2 | knockout | CAGAATACAACTCAAGCAGG |
| | | | (SEQ ID NO: 21) |
| CTAG1A/B | sg3 | knockout | GAATGGATGCTGCAGATGCG |
| | | | (SEQ ID NO: 22) |
| CD274 | sg1 | activation | CTGACCTTCGGTGAAATCGG |
| | | | (SEQ ID NO: 23) |
| CD274 | sg2 | activation | TCAGTTTAGGTATCTAGTGT |
| | | | (SEQ ID NO: 24) |
| CD274 | sg3 | activation | CTATACACAGCTTTATTCCT |
| | | | (SEQ ID NO: 25) |
| MCL1 | sg1 | activation | CATGGAAAGAGCTCGAGCCC |
| | | | (SEQ ID NO: 26) |
| MCL1 | sg2 | activation | CACTCAGAGCCTCCGAAGAC |
| | | | (SEQ ID NO: 27) |
| MCL1 | sg3 | activation | CGGAGCCGCCGTTACGTAAC |
| | | | (SEQ ID NO: 28) |
| JUNB | sg1 | activation | CCCCTCCTCGAGCGTGGGGA |
| | | | (SEQ ID NO: 29) |
| JUNB | sg2 | activation | AGGCGGCTCGCGTCACTGTC |
| | | | (SEQ ID NO: 30) |
| JUNB | sg3 | activation | GCGCGTGTCCTTGTAAACAG |
| | | | (SEQ ID NO: 31) |
| B3GNT2 | sg1 | activation | GCCGCAGGGAGCGCGGGCCC |
| | | | (SEQ ID NO: 32) |
| B3GNT2 | sg2 | activation | GTGGGTCCTGGTACCGGGTG |
| | | | (SEQ ID NO: 33) |
| B3GNT2 | sg3 | activation | CGGAACCCTCCCAAAACTTG |
| | | | (SEQ ID NO: 34) |
| CD274 | sg1 | knockdown | AGCAGCTGGCGCGTCCCGCG |
| | | | (SEQ ID NO: 35) |
| CD274 | sg2 | knockdown | TCGGGAAGCTGCGCAGAACT |
| | | | (SEQ ID NO: 36) |
| MCL1 | sg1 | knockdown | AGCTTCCGGAGGGTTGCGCA |
| | | | (SEQ ID NO: 37) |
| MCL1 | sg2 | knockdown | CCTTTATCACGGTTTTAGGG |
| | | | (SEQ ID NO: 38) |
| JUNB | sg1 | knockdown | CTGGGACCTTGAGAGCGGCC |
| | | | (SEQ ID NO: 39) |
| JUNB | sg2 | knockdown | TATCGCGCCAGAGAGGGCGA |
| | | | (SEQ ID NO: 40) |
| B3GNT2 | sg1 | knockdown | CTGCGCCTCACTCCAGGCTC |
| | | | (SEQ ID NO: 41) |
| B3GNT2 | sg2 | knockdown | GGAGTGAGGCGCAGCGGCAG |
| | | | (SEQ ID NO: 42) |
| BCL2A1 | sg1 | knockdown | TACGCACGAAAGTGACTAGG |
| | | | (SEQ ID NO: 43) |
| BCL2A1 | sg2 | knockdown | ACATGATGATACATGGAGGC |
| | | | (SEQ ID NO: 44) |
| BCL2A1 | sg3 | knockdown | GGCTCACCTTGAAGCTGTTG |
| | | | (SEQ ID NO: 45) |
| BCL2A1 | sg4 | knockdown | TCAAGACTTTGCTCTCCACC |
| | | | (SEQ ID NO: 46) |
| CD276 | sg1 | knockdown | GCGGCTCCGGTGCGTCCCTG |
| | | | (SEQ ID NO: 47) |
| CD276 | sg2 | knockdown | GCGTCCCTGAGTCCCAGAGT |
| | | | (SEQ ID NO: 48) |
| CD274 | sg1 | knockout | ACATGTCAGTTCATGTTCAG |
| | | | (SEQ ID NO: 49) |
| CD274 | sg2 | knockout | GGTTCCCAAGGACCTATATG |
| | | | (SEQ ID NO: 50) |
| MCL1 | sg1 | knockout | AGTCGCTGGAGATTATCTCT |
| | | | (SEQ ID NO: 51) |
| MCL1 | sg2 | knockout | CCAAAAGTCGCCCTCCCGGG |
| | | | (SEQ ID NO: 52) |
| JUNB | sg1 | knockout | CCGGAGTCTCAAAGCGCCTG |
| | | | (SEQ ID NO: 53) |
| JUNB | sg2 | knockout | GGGTAAAAGTACTGTCCCGG |
| | | | (SEQ ID NO: 54) |
| B3GNT2 | sg1 | knockout | CAACGCAGGGAACCAAACGG |
| | | | (SEQ ID NO: 55) |
| B3GNT2 | sg2 | knockout | GGTTCCAGTATGCCTCGGGA |
| | | | (SEQ ID NO: 56) |
| TNFSF9 | sg1 | knockout | CCCATCGATCAGCAGAACTG |
| | | | (SEQ ID NO: 57) |
| TNFSF9 | sg2 | knockout | GCCAGCCCGAGACTCCGCGA |
| | | | (SEQ ID NO: 58) |
| TNFSF9 | sg3 | knockout | GGGGGGCCTGAGCTACAAAG |
| | | | (SEQ ID NO: 59) |
| TNFSF9 | sg4 | knockout | TCAACTAGAGCTGCGGCGCG |
| | | | (SEQ ID NO: 60) |
| Non- | sg1 | | CTGAAAAAGGAAGGAGTTGA |
| targeting | | | (SEQ ID NO: 61) |
| Non- | sg2 | | AAGATGAAAGGAAAGGCGTT |
| targeting | | | (SEQ ID NO: 62) |

### Cell Culture

HEK293FT cells (Thermo Fisher Scientific R70007) were maintained in high-glucose DMEM with GlutaMax and pyruvate (Thermo Fisher Scientific 10569010) supplemented with 10% fetal bovine serum (VWR 97068-085) and 1% penicillin/streptomycin (Thermo Fisher Scientific 15140122). Cells were passaged every other day at a ratio of 1:4 or 1:5 using TrypLE Express (Thermo Fisher Scientific 12604021).

All cancer cell lines [A375 melanoma (NY-ESO-1+, HLA-A2+; Millipore Sigma 88113005-1VL), H1793 non-small cell lung adenocarcinoma (NY-ESO-1+, HLA-A2-; ATCC CRL-5896), H1299 non-small cell lung carcinoma (NY-ESO-1+, HLA-A2; ATCC CRL-5803), LN-18 glioblastoma (NY-ESO-1+, HLA-A2+; ATCC CRL-2610), SK-N-AS neuroblastoma (NY-ESO-1+, HLA-A2; ATCC CRL-2137), A2058 melanoma (NY-ESO-1⁺, HLA-A2; ATCC CRL-11147), OAW28 ovarian cystadenocarcinoma (NY-ESO-1⁺, HLA-A2; Millipore Sigma 85101601-1VL), and SW1417 colorectal adenocarcinoma (NY-ESO-1, HLA-A2; ATCC CCL-238)] were maintained in RPMI 1640 with Glutamax (Thermo Fisher Scientific 61870127) supplemented with 10% fetal bovine serum and 1% penicillin/streptomycin. Cells were passaged every other day at a ratio of 1:3 to 1:6 using TrypLE Express.

Leukopaks from anonymous healthy normal donors (purchased from MGH under an IRB-exempt protocol) are processed using the Ficoll-based RosetteSep Human T Cell Enrichment Cocktail (StemCell Technologies 15061). Isolated CD4⁺ and CD8+ T cells were frozen in FBS with 10% DMSO with 20-50×10⁶ cells per vial. Once thawed, T cells were maintained in RPMI 1640 with Glutamax (Thermo Fisher Scientific 61870127) supplemented with 10% fetal bovine serum, 1% penicillin/streptomycin, and 20 IU/mL IL-2 (Stemcell Technologies 78036.3). T cells were activated and expanded for 1 week using CD3/CD28 Dynabeads (Thermo Fisher Scientific 11132D). Beads were removed with 2 rounds of magnetic separation and T cells were frozen down (for in vitro cytotoxicity assays) or cultured for 1 week without beads (for adoptive cell transfer). CD4⁺ or CD8⁺ T cells were further purified using EasySep selection kits (StemCell Technologies 17852 and 17853 respectively) to assess the resistance of candidate genes against cytotoxicity produced from each T cell type. Each experiment with T cells was performed using T cells derived from 2-4 unique donors.

### Lentivirus Production and Transduction

One day prior to transfection, HEK293FT cells were seeded at ˜40% confluency in T25, T75, or T225 flasks (Thermo Fisher Scientific 156367, 156499, or 159934). Cells were transfected the next day at ˜90-99% confluency. For each T25 flask, 3.4 μg of plasmid containing the vector of interest, 2.6 μg of psPAX2 (Addgene 12260), and 1.7 μg of pMD2.G (Addgene 12259) were transfected using 17.5 μL of Lipofectamine 3000 (Thermo Fisher Scientific L3000150), 15 μL of P3000 Enhancer (Thermo Fisher Scientific L3000150), and 1.25 mL of Opti-MEM (Thermo Fisher Scientific 31985070). Transfection parameters were scaled up linearly with flask area for T75 and T225 flasks. Media was changed 5 h after transfection. Virus supernatant was harvested 48 h post-transfection, filtered with a 0.45 μm PVDF filter (MilliporeSigma SLHV013SL), and concentrated as described previously when necessary (Joung et al., 2017). Virus supernatant was then aliquoted and stored at −80° C.

Cancer cell lines were transduced by spinfection or mixing as described previously (Joung et al., 2017). For mixing, 3×10⁶ cells were seeded in a T75 flask with 8 μg/mL Polybrene (Millipore Sigma TR-1003-G) and the appropriate volume in lentivirus. After 1 day, media was refreshed with the appropriate antibiotic and cells were maintained under antibiotic selection for 5 days. Concentrations for selection agents were determined using a kill curve: 300 μg/mL Hygromycin (Thermo Fisher Scientific 10687010), 10 μg/mL Blasticidin (Thermo Fisher Scientific A1113903), 300 μg/mL Zeocin (Thermo Fisher Scientific R25001), and 1 μg/mL Puromycin (Thermo Fisher Scientific A1113803). T cells were transduced after 1 day of activation by mixing 1×10⁶ cells in 1 mL media with 8 μg/mL Polybrene and lentivirus in each well of a 24-well plate (Millipore Sigma CLS3527-100EA). Transduction efficiency of T cells was measured by sorting 1×10⁶ cells for GFP expression on the TCR vector after 7 days of activation. T cells used for experiments had transduction efficiencies of 80-90%.

### T Cell Cytotoxicity Assays

Expanded T cells were thawed and maintained in culture media for 8-10 h before incubation with cancer cells. Cancer cells were seeded in 96-well plates and allowed to attach for 3-4 h before T cells were added at the appropriate effector to target cell (E:T) ratio. Paired controls with no T cells added were included for each condition. After 18 h, cancer cells were washed twice with PBS to remove T cells, passaged, and cultured for 2 days. Primary patient-derived cell models were not passaged after T cell co-culture. Viability was measured using CellTiter-Glo (Promega G7571). For each E:T ratio, percent survival was calculated as viability of the cells incubated with T cells divided by viability of the paired control that was not incubated with T cells. For example, CD274-overexpressing melanoma cells that were co-cultured with ESO T cells were compared to CD274-overexpressing melanoma cells that were cultured without T cells in parallel. As an alternative cytotoxicity assay, A375 cells stably expressing Gaussia luciferase were co-cultured with ESO T cells. At each time point, 10% of cell culture media was used for the Gaussia luciferase assay (Targeting Systems GAR-2B) to directly measure cytotoxicity.

### CRISPRa Screen for Resistance to T Cell Cytotoxicity

The CRISPRa screen was performed as described previously (Joung et al., 2017) using A375 melanoma cells. For the acute exposure screen, A375 cells transduced with the genome-scale human CRISPR activation library components were co-cultured with T cells expressing the NY-ESO-1 TCR, unmodified T cells, or no T cells at E:T ratio of 3. Each screen contained two replicates with T cells from different donors. After 18 h of co-culture, cells were washed twice with PBS to remove T cells, passaged, and cultured for 2 days before genomic DNA was harvested. For the chronic exposure screen, A375 cells were co-cultured with T cells expressing the NY-ESO-1 TCR or no T cells at E:T ratio of 2. Screening replicates used T cells from the same donor and each round of screening selection used T cells from different donors. After 3 days of co-culture, cells were washed twice with PBS to remove T cells, passaged, and cultured for 2 days before seeding for the next round of screening selection. After 3 rounds of screening selection, genomic DNA was harvested. MAGeCK RRA analysis (Li et al., 2014) was used to analyze the screens and identify candidate genes. A set of 576 candidate genes that ranked in the top 1% and overlapped at least two screening replicates (combining the acute and chronic exposure screens) were used for pathway and cytolytic activity analyses. The FDR of screening results was estimated using a set of 311 negative control housekeeping genes consisting of ribosomal proteins, RNA polymerases, translation factors, mitochondrial ribosomal proteins, GAPDH, and ACTB (Table 2). For each screening replicate, the FDR of each candidate gene was measured as the fraction of negative control genes with higher average sgRNA enrichment than the candidate gene. To validate the top four candidate genes from the screens, sgRNAs targeting candidate genes from the genome-scale library were individually cloned and transduced into A375 cells. Validation was performed using T cell cytotoxic assays at an E:T ratio of 3 as described above.

### Pathway Enrichment Analysis

Pathway enrichment analysis of the top 576 candidate genes was performed using g:Profiler (Raudvere et al., 2019). GO:BP pathways with between 5 and 200 genes that were significantly enriched (FDR<0.05) were included. To identify non-overlapping pathways, the enriched pathways were sorted by FDR and any pathway that had more than 30% genes overlapping a different pathway with lower FDR was excluded.

### The Cancer Genome Atlas (TCGA) Analysis

TCGA copy number variation and RNA-seq data was downloaded from the Firehose Broad GDAC (gdac.broadinstitute.org/) using the TCGA2STAT package for R (Wan et al., 2016). The RNA-seq data was normalized using RSEM and log 2 transformed. Local tumor immune cytolytic activity was determined as the geometric mean of granzyme A (GZMA) and perforin 1 (PRF1) RNA-seq expression was used to assess cytolytic activity as described previously (Patel et al., 2017; Rooney et al., 2015). For each gene in the TCGA RNA-seq dataset, the Pearson's correlation between cytolytic activity and expression was calculated. Significance was evaluated using Fisher transformation of Pearson's correlation followed by Benjamini-Hochberg procedure to determine the FDR. For visualization, heatmaps with hierarchical clustering using Ward's linkage were generated using Python's Seaborn clustermap (github.com/mwaskom/seaborn/).

For prevalence of increased expression and copy number of the top four candidate genes, TCGA RNA-seq data (https://www.cancer.gov/tcga) was analyzed using GEPIA (Tang et al., 2017). TCGA tumor samples were matched with TCGA normal and GTEx data and filtered for |log 2(fold change)|≥1. Genes were considered significantly differentially expressed if the p-value was greater than 0.05 FDR correction. Copy number variation was reported using the NCI Genomic Data Commons (Grossman et al., 2016).

### Indel Analysis

Cells plated in 96-well plates were grown to 60-80% confluency and assessed for indel rates as previously described (Joung et al., 2017). Genomic DNA was harvested from cells using QuickExtract DNA Extraction kit (Lucigen QE09050). The genomic region flanking the site of interest was amplified using NEBNext High Fidelity 2×PCR Master Mix (New England BioLabs M0541L), first with region-specific primers (Table 9) for 15 cycles and then with barcoded primers for 15 cycles as previously described. PCR products were sequenced on the Illumina MiSeq platform (>10,000 reads per condition), and indel analysis was performed as previously described (Joung et al., 2017).

TABLE 9
Primers for indel amplification and quantification.

| Target gene | sgRNA ID | Primer | Sequence (5′ to 3′) |
|---|---|---|---|
| CTAG1A/B | sg1 | Fwd | CTTTCCCTACACGACGCTCTTCCGATCTGGGCAGCAAG |
| | | | GGCCTC (SEQ ID NO: 63) |
| CTAG1A/B | sg1 | Rev | GACTGGAGTTCAGACGTGTGCTCTTCCGATCTGCTCTC |
| | | | CGGCCCCCT (SEQ ID NO: 64) |
| CTAG1A/B | sg2 | Fwd | CTTTCCCTACACGACGCTCTTCCGATCTTCAGGGCTGA |
| | | | ATGGATGCTG (SEQ ID NO: 65) |
| CTAG1A/B | sg2 | Rev | GACTGGAGTTCAGACGTGTGCTCTTCCGATCTTGCCCT |
| | | | CCCCATCTCCC (SEQ ID NO: 66) |
| CTAG1A/B | sg3 | Fwd | CTTTCCCTACACGACGCTCTTCCGATCTGGGCAGCAAG |
| | | | GGCCTC (SEQ ID NO: 67) |
| CTAG1A/B | sg3 | Rev | GACTGGAGTTCAGACGTGTGCTCTTCCGATCTGCCCCC |
| | | | ACCTCGCCA (SEQ ID NO: 68) |
| CD274 | sg1 | Fwd | CTTTCCCTACACGACGCTCTTCCGATCTTGTTTATGTC |
| | | | CTAGCCCCATAC (SEQ ID NO: 69) |
| CD274 | sg1 | Rev | GACTGGAGTTCAGACGTGTGCTCTTCCGATCTCTTGAT |
| | | | GGTCACTGCTTGTCC (SEQ ID NO: 70) |
| CD274 | sg2 | Fwd | CTTTCCCTACACGACGCTCTTCCGATCTAAACGCTGTG |
| | | | CCAATTTTGTAAATG (SEQ ID NO: 71) |
| CD274 | sg2 | Rev | GACTGGAGTTCAGACGTGTGCTCTTCCGATCTGACAAT |
| | | | TAGTGCAGCCAGGTCTA (SEQ ID NO: 72) |
| MCL1 | sg1 | Fwd | CTTTCCCTACACGACGCTCTTCCGATCTGGAGTTGGTC |
| | | | GGGGAATCTG (SEQ ID NO: 73) |
| MCL1 | sg1 | Rev | GACTGGAGTTCAGACGTGTGCTCTTCCGATCTCAACCC |
| | | | GTCGTAAGGTCTCC (SEQ ID NO: 74) |
| MCL1 | sg2 | Fwd | CTTTCCCTACACGACGCTCTTCCGATCTAAAAGAAACG |
| | | | CGGTAATCGGAC (SEQ ID NO: 75) |
| MCL1 | sg2 | Rev | GACTGGAGTTCAGACGTGTGCTCTTCCGATCTGCGCTT |
| | | | CCGCCAATCAC (SEQ ID NO: 76) |
| JUNB | sg1 | Fwd | CTTTCCCTACACGACGCTCTTCCGATCTGGCCTCTCTC |
| | | | TACACGACTAC (SEQ ID NO: 77) |
| JUNB | sg1 | Rev | GACTGGAGTTCAGACGTGTGCTCTTCCGATCTCAGCTC |
| | | | CGAAGAGGCGAG (SEQ ID NO: 78) |
| JUNB | sg2 | Fwd | CTTTCCCTACACGACGCTCTTCCGATCTATTGTCCCCA |
| | | | ACAGCAACGG (SEQ ID NO: 79) |
| JUNB | sg2 | Rev | GACTGGAGTTCAGACGTGTGCTCTTCCGATCTTTGTGC |
| | | | AGATCGTCCAGGGC (SEQ ID NO: 80) |
| B3GNT2 | sg1 | Fwd | CTTTCCCTACACGACGCTCTTCCGATCTTGCTGGCGAT |
| | | | TAAGTCCCTC (SEQ ID NO: 81) |
| B3GNT2 | sg1 | Rev | GACTGGAGTTCAGACGTGTGCTCTTCCGATCTATCTGA |
| | | | AAGGTCGGGGTGGT (SEQ ID NO: 82) |
| B3GNT2 | sg2 | Fwd | CTTTCCCTACACGACGCTCTTCCGATCTTCTCCAAAAG |
| | | | CAGTAGCCAAG (SEQ ID NO: 83) |
| B3GNT2 | sg2 | Rev | GACTGGAGTTCAGACGTGTGCTCTTCCGATCTTATATT |
| | | | GGAGAGCCTGCCCG (SEQ ID NO: 84) |
| TNFSF9 | sg1 | Fwd | CTTTCCCTACACGACGCTCTTCCGATCTTCTTTTCTCC |
| | | | CAGGGCTGC (SEQ ID NO: 85) |
| TNFSF9 | sg1 | Rev | GACTGGAGTTCAGACGTGTGCTCTTCCGATCTCTTTGT |
| | | | AGCTCAGGCCCCC (SEQ ID NO: 86) |
| TNFSF9 | sg2 | Fwd | CTTTCCCTACACGACGCTCTTCCGATCTTTCCTCGCCT |
| | | | GCCCCT (SEQ ID NO: 87) |
| TNFSF9 | sg2 | Rev | GACTGGAGTTCAGACGTGTGCTCTTCCGATCTGCCGCA |
| | | | GGTCCAAGAGG (SEQ ID NO: 88) |
| TNFSF9 | sg3 | Fwd | CTTTCCCTACACGACGCTCTTCCGATCTACAAAGAGGA |
| | | | CACGAAGGAGC (SEQ ID NO: 89) |
| TNFSF9 | sg3 | Rev | GACTGGAGTTCAGACGTGTGCTCTTCCGATCTCCCCAG |
| | | | CAGCAGAGCG (SEQ ID NO: 90) |
| TNFSF9 | sg4 | Fwd | CTTTCCCTACACGACGCTCTTCCGATCTTTTCCTCCCA |
| | | | CAGTTCTGCTGAT (SEQ ID NO: 91) |
| TNFSF9 | sg4 | Rev | GACTGGAGTTCAGACGTGTGCTCTTCCGATCTCGCAGC |
| | | | TCTAGTTGAAAGAAGACA (SEQ ID NO: 92) |

qPCR Quantification of Transcript Expression

Cells were seeded in 96-well plates and grown to 60-90% confluency before RNA was reverse transcribed for qPCR as described previously (Joung et al., 2017). TaqMan qPCR was performed with custom [B3GNT2-Fwd (GGGCAGGCTCTCCAATATAAG (SEQ ID NO: 93)), B3GNT2-probe (/56-FAM/TGAACTACT/Zen/GCGAACCTGACCTGA/3IABKFQ/(SEQ ID NO: 94)), B3GNT2-Rev (GGCATCTCAAATACAGCAGAAAG (SEQ ID NO: 95))] or readymade probes from Thermo Fisher Scientific [CD274 (Hs00204257_m1), MCL1 (Hs01050896_ml), JUNB (Hs00357891_s1), BID (Hs00609632_m1), PMAIP1 (Hs00560402_m1), BBC3 (Hs00248075_m1), BAD (Hs00188930_m1), BAX (Hs00180269_m1), BAKI (Hs00832876_g1), BCL2A1 (Hs06637394_s1), CD276 (Hs00987207_m1)].

### Adoptive Cell Transfer and In Vivo Validation

The designs of animal studies and procedures were approved by the Institutional Animal Care and Use Committee (IACUC) of the Broad Institute. Ethical compliance with IACUC protocols and institute standards was maintained. Specific pathogen-free facilities at the Broad Institute was used for the storage and care of all mice. Female NSG mice (strain 005557) aged 4-6 weeks were purchased from The Jackson Laboratory and used for tumor induction experiments. A375 cells were transduced with dox-inducible candidate genes. NSG mice were subcutaneously injected with 1×10⁶ A375 cells. After 2 days of tumor xenograft implantation, mice were switched to 1,000 mg/kg doxycycline diet (Envigo TD.05298). At 7 days after tumor implantation, for the adoptive cell transfer conditions, 2×10⁷ ESO T cells were intravenously injected in a blinded manner. Each tumor was measured every 2 days beginning on day 7 after ACT until the survival endpoint was reached. Measurements were assessed manually using the longest dimension (length) and the longest perpendicular dimension (width). Tumor volume was estimated with the formula: (L×W²)/2. Mice with tumor volumes greater than 2,000 mm³ were euthanized. CO₂ inhalation was used to euthanize mice. No statistical methods were used to predetermine sample size. Sample size was determined based on prior knowledge of the variability of experiments with ACT. Animals were randomized before treatment and no blinding was performed for tumor measurements.

### Bulk RNA Sequencing and Data Analysis

RNA from cells plated in 24-well plates and grown to 60-90% confluency was harvested using the RNeasy Plus Mini Kit (Qiagen 74134). RNA-seq libraries were prepared using NEBNext Ultra RNA Library Prep Kit for Illumina (New England Biolabs E7530S) and deep sequenced on the Illumina NextSeq platform (>9 million reads per biological replicate). Bowtie (Langmead et al., 2009) index was created based on the human hg38 UCSC genome and RefSeq transcriptome. Next, RSEM v1.3.1 (Li and Dewey, 2011) was run with command line options “--estimate-rspd--bowtie-chunkmbs 512--paired-end” to align paired-end reads directly to this index using Bowtie and estimate expression levels in transcripts per million (TPM) based on the alignments.

To identify genes that were differentially expressed as a result of ORF overexpression, RSEM's TPM estimates for each transcript were transformed to log-space by taking log 2(TPM+1). Transcripts were considered detected if their expression level was equal to or above 10. All genes detected in at least three libraries were used to find differentially expressed genes. The Student's t-test was performed on the TF ORF overexpression condition against GFP control condition. Only genes that were significant (p-value pass 0.01 FDR correction) were reported.

Chromatin Immunoprecipitation with Sequencing (ChIP-Seq)

Cells were plated in 10-cm cell culture dishes and grown to 60-80% confluency. For each condition, two biological replicates were harvested for ChIP-seq. Formaldehyde (Millipore Sigma 252549) was added directly to the growth media for a final concentration of 1% and cells were incubated at 37° C. for 10 mins to initiate chromatin fixation. Fixation was quenched by adding 2.5 M glycine (Millipore Sigma G7126) in PBS for a final concentration of 125 mM glycine and incubated at room temperature for 5 mins. Cells were then washed with ice-cold PBS, scraped, and pelleted at 1,000×g for 5 mins.

Cell pellets were prepared for ChIP-seq using the Epigenomics Alternative Mag Bead ChIP Protocol v2.0 (Consortium, 2004). Briefly, cell pellets were resuspended in 100 μL of lysis buffer (1% SDS, 10 mM EDTA, 50 mM Tris-HCL pH 8.1) containing protease inhibitor cocktail (Millipore Sigma 05892791001) and incubated for 10 mins at 4° C. Then 400 μL of dilution buffer (0.01% SDS, 1.1% Triton X-100, 1.2 mM EDTA, 16.7 mM Tris-HCl pH 8.1, and 167 mM NaCl) containing protease inhibitor cocktail was added. Samples were pulse sonicated with 2 rounds of 10 mins (30s on-off cycles, high frequency) in a rotating water bath sonicator (Diagenode Bioruptor) with 5 mins on ice between each round. 10 μL of sonicated sample was set aside as input control. Then 500 μL of dilution buffer (0.01% SDS, 1.1% Triton X-100, 1.2 mM EDTA, 16.7 mM Tris-HCl pH 8.1, and 167 mM NaCl) containing protease inhibitor cocktail and 1 μL of anti-FLAG (Millipore Sigma F3165-1MG) was added to the sonicated sample. ChIP samples were rotated end over end overnight at 4° C.

For each ChIP, 50 μL of Protein A/G Magnetic Beads (Thermo Fisher Scientific 88802) was washed with 1 mL of blocking buffer (0.5% TWEEN and 0.5% BSA in PBS) containing protease inhibitor cocktail twice before resuspending in 100 μL of blocking buffer. ChIP samples were transferred to the beads and rotated end over end for 1 h at 4° C. ChIP supernatant was then removed and the beads were washed twice with 200 μL of RIPA low salt buffer (0.1% SDS, 1% Triton x-100, 1 mM EDTA, 20 mM Tris-HCl pH 8.1, 140 mM NaCl, 0.1% DOC), twice with 200 μL of RIPA high salt buffer (0.1% SDS, 1% Triton x-100, 1 mM EDTA, 20 mM Tris-HCl pH 8.1, 500 mM NaCl, 0.1% DOC), twice with 200 μL of LiCI wash buffer (250 mM LiCl, 1% NP40, 1% DOC, 1 mM EDTA, 10 mM Tris-HCl pH 8.1), and twice with 200 μL of TE (10 mM Tris-HCl pH8.0, 1 mM EDTA pH 8.0). ChIP samples were eluted with 50 μL of elution buffer (10 mM Tris-HCl pH 8.0, 5 mM EDTA, 300 mM NaCl, 0.1% SDS). 40 μL of water was added to the input control samples. 8 μL of reverse cross-linking buffer (250 mM Tris-HCl pH 6.5, 62.5 mM EDTA pH 8.0, 1.25 M NaCl, 5 mg/ml Proteinase K, 62.5 μg/ml RNAse A) was added to the ChIP and input control samples and then incubated at 65ºC for 5 h. After reverse crosslinking, samples were purified using 116 μL of SPRIselect Reagent (Beckman Coulter B23318).

ChIP samples were prepared for NGS with NEBNext Ultra II DNA Library Prep Kit for Illumina (New England Biolabs E7645S) and deep-sequenced on the Illumina NextSeq platform (>60 million reads per condition). Bowtie (Langmead et al., 2009) was used to align paired-end reads to the human hg38 UCSC genome with command line options q-X 300—sam —chunkmbs 512″. Next, biological replicates were merged and Model-based Analysis of ChIP-seq (MACS) (Feng et al., 2012) was run with command line options “-g hs-B-S—mfold 6,30” to identify TF peaks. HOMER (Heinz et al., 2010) was used to discover motifs in the TF peak regions identified by MACS. TFs were considered potential regulators of a candidate gene if the TF peak region identified by MACS overlapped with the 20 kb region centered around the transcriptional start site of the candidate gene based on RefSeq annotations.

### Co-Immunoprecipitation (Co-IP) and Mass Spectrometry

Cells were plated in 10-cm cell culture dishes and grown to 60-80% confluency. For each condition, two biological replicates were harvested for co-IP. Cells were washed with PBS and 4 mL of lysis buffer (20 mM HEPES, 1% Triton X-100, 150 mM NaCl, 1 mM EDTA, and 10% glycerol) containing protease inhibitor cocktail was added. Cells were scraped and lysate was incubated at 4° C. under rotary agitation for 1 h. Lysate was centrifuged at 14,000×g for 10 min at 4° C. Supernatant was transferred to a new tube and an aliquot was taken as the input. The remaining lysate was split into two tubes for the FLAG and IgG control conditions. For mass spectrometry, 10 μg/mL Mouse Anti-FLAG (Millipore Sigma F3165-1MG) and IgG control (Millipore Sigma 12-371) were added to the respective conditions. For Western blots, 10 μg/mL Chicken Anti-FLAG (Aves labs ET-DY100) and IgY control (R&D Systems AB-101-C) antibodies were biotinylated (Thermo Fisher Scientific 90407) and added to the respective conditions. Lysates with antibodies were incubated at 4° C. under rotary agitation overnight. For each mL of lysate, 50 μL of Pierce Protein A/G Magnetic Beads (Mass spectrometry; Thermo Fisher Scientific 88803) or Pierce Streptavidin Magnetic Beads (Western blot; Thermo Fisher Scientific 88817) was washed twice with lysis buffer. Lysates with antibodies were added to the beads and incubated at 4° C. under rotary agitation for 4 h. Beads were washed with lysis buffer 3 times and resuspended in lysis buffer for storage.

Magnetic beads were resuspended in 100 mM Tris pH 7.8, reduced, alkylated and digested with trypsin at 37° ° C. overnight. This solution was subjected to solid phase extraction to concentrate the peptides and remove unwanted reagents followed by injection onto a Shimadzu HPLC with fraction collector. Eight fractions were collected and after concentration were injected on a Waters NanoAcquity HPLC equipped with a self-packed Aeris 3 μm C18 analytical column 0.075 mm by 20 cm, (Phenomenex). Peptides were eluted using standard reverse-phase gradients. The effluent from the column was analyzed using a Thermo Orbitrap Elite mass spectrometer (nanospray configuration) operated in a data dependent manner for 54 minutes. The resulting fragmentation spectra were correlated against the known database using Proteome Discover 1.4 (Thermo Fisher Scientific). Scaffold Q+S (Proteome Software) was used to provide consensus reports for the identified proteins.

### Cytokine Assays

To challenge cells with cytokines, cells were incubated with Interferon-γ (IFNγ; Cell Signaling Technology 80385S), FasL (AdipoGen AG-40B-0130-3010), TRAIL (R&D Systems 375-TL-010), or TNF-α (AdipoGen AG-40B-0019-3010) for 24 h. TRAIL was crosslinked by incubating with anti-His Tag antibody (Thermo Fisher Scientific MA121315, 1:500) for 15 min at room temperature. Cell viability was measured using CellTiter-Glo (Promega G7571) and protein was harvested for Western blots. For evaluating Caspase 8 activity, cells were incubated with FasL or crosslinked TRAIL for 3 h and harvested for Western blot or Caspase 8 colorimetric assay (R&D Systems K113-100). IFNγ in the cell culture media of the T cell cytotoxic assay was quantified using an ELISA kit (Thermo Fisher Scientific KHC4021).

### Small Molecule Inhibition

For glycosylation inhibition, cells were treated with 20 μg/mL Kifunensine (Cayman Chemical 10009437) or 2 mM Benzyl 2-acetamido-2-deoxy-alpha-D-galactopyranoside (BAG; Millipore Sigma B4894-100MG) for 48 h to inhibit N- and O-glycosylation respectively unless otherwise indicated. For MCL1 inhibition, cells were treated with 1-10 μM of S63845 (Selleck Chemicals S8383) or AZD5991 (Selleck Chemicals S8643) for 4 h before incubation with T cells. Both glycosylation and MCL1 inhibitors were maintained at indicated concentrations during co-culture with T cells.

### Western Blot

Protein lysates were harvested with RIPA lysis buffer (Cell Signaling Technologies 9806S) containing protease inhibitor cocktail (MilliporeSigma 05892791001). Samples were standardized for protein concentration using the Pierce BCA protein assay (VWR 23227), and incubated at 70ºC for 10 mins under reducing conditions. To determine presence of glycosylation, samples were treated with Protein Deglycosylation Mix II (O- and N-deglycosylation; New England Biolabs P6044S) or PNGase F (N-deglycosylation; New England Biolabs P0704L). After denaturation, samples were separated by Bolt 4-12% Bis-Tris Plus Gels (Thermo Fisher Scientific NW04125BOX) and transferred onto a PVDF membrane using iBlot Transfer Stacks (Thermo Fisher Scientific IB401001).

Blots were blocked with 5% BLOT-QuickBlocker (G Biosciences 786-011) in TBST for 1 h at room temperature. Blots were then probed with different primary antibodies [phospho-NF-κB p65 Ser536 (Cell Signaling Technology 3033S, 1:1,000), NF-κB p65 (Santa Cruz Biotechnology sc-8008, 1:200), phospho-STAT1 Tyr701 (Cell Signaling Technology 9167S, 1:1,000), STAT1 (Cell Signaling Technology 9172S, 1:1,000), CD276 (R&D Systems AF1027, 1:200), CD70 (Santa Cruz Biotechnology sc-365539, 1:200), CD58 (Thermo Fisher Scientific MA5800, 1:200), NECTIN2 (R&D Systems AF2229, 1:2,000), HLAA (Abcam ab52922, 1:5,000), TNFRSF1A (Santa Cruz Biotechnology sc-8436, 1:200), IFNGR2 (R&D Systems AF773, 1:200), FAS (Santa Cruz Biotechnology sc-8009, 1:200), IFNARI (Santa Cruz Biotechnology sc-7391, 1:100), TNFRSF10B (Novus Biologicals NB100-56618, 1:200), MICB (R&D Systems MAB1599-100, 1:500), TNFRSF10A (R&D Systems AF347, 1:200), PVR (R&D Systems MAB25301, 1:500), MICA (R&D Systems MAB1300-100, 1:500), HMGB1 (Abcam ab18256, 1:1,000), 4-1BBL (TNFSF9; R&D Systems AF2295, 1:200), NT5E (Abcam ab175396, 1:1,000), ULBP2 (R&D Systems AF1298, 1:2,000), IFNGR1 (R&D Systems MAB6731, 1:500), ULBP3 (R&D Systems AF1517, 1:2,000), CD39 (Abcam ab108248, 1:1,000), FLAG (Millipore Sigma F7425, 1:1,000), or GAPDH (Cell Signaling Technology 2118L, 1:1,000)] in 2.5% BLOT-QuickBlocker (G Biosciences 786-011) in TBST overnight at 4° C. Blots were washed with TBST before incubation with secondary antibodies [Anti-rabbit IgG, HRP-linked (Cell Signaling Technology 7074S, 1:5,000), Anti-mouse IgG, HRP-linked (Cell Signaling Technology 7076S, 1:5,000), anti-goat IgG-HRP (Santa Cruz Biotechnology sc-2354, 1:5,000)] in 2.5% BLOT-QuickBlocker (G Biosciences 786-011) in TBST for 1 h at room temperature. Blots were washed with TBST and imaged using chemiluminescent substrate [Pierce ECL (Thermo Fisher Scientific 32209), SuperSignal West Pico PLUS (Thermo Fisher Scientific 34577), or SuperSignal West Femto (Thermo Fisher Scientific 34096)] on the ChemiDox XRS+ (Bio-Rad).

Caspase 8 blots were transferred onto a PVDF membrane with traditional wet transfer at 50V for 1 h. Blots were blocked with 5% bovine serum albumin (BSA; Millipore Sigma A9418) in TBST for 2 h at room temperature before probing with Caspase 8 primary antibody (Cell Signaling Technology 9746S, 1:1,000) in 5% BSA in TBST overnight at 4° C. Blots were washed with TBST before incubation with anti-mouse IgG, HRP-linked secondary antibody in 5% BSA in TBST for 1 h at room temperature. Blots were washed with TBST and imaged as described above.

### Flow Cytometry Assays

Per condition, 5×10⁵ cells were pelleted at 200×g for 5 min and washed once with PBS. Cell were fixed in 4% Formaldehyde in PBS at 4° C. for 10 mins. Cells were washed twice with PBS and resuspended in PBS with 25 μg/mL recombinant Fc chimera proteins [PVRIG (R&D Systems 9365-PV-050), CD226 (R&D Systems 666-DN-050), NKG2D (R&D 1299-NK-050), TREML2 (R&D Systems 3259-TL-050), CD2 (R&D Systems 1856-CD-050), CD96 (R&D Systems 9360-CD-050), TIGIT (BPS Bioscience 71186), CD27 (BPS Bioscience 71176), or 4-1BB (TNFRSF9; Sino Bio 10041-H03H)], 0.1 μg/mL HLA-A2:NY-ESO-1 Fab (Stewart-Jones et al., 2009), 5 μg/mL Fas antibody (Millipore Sigma 05-201), 25 μg/mL TNFRSF10B antibody (Novus Biologicals NB100-56618, 1:200), or Dylight 649 labeled Tomato Lectin (Vector Laboratories DL-1178, 1:100). Cells were incubated at 4° C. for 1 h. Cells were washed twice with PBS and resuspended in PBS with the appropriate secondary antibody [IgG Fc PE (Thermo Fisher Scientific 12-4998-82, 1:50), His Tag Alexa Fluor 647 (Thermo Fisher Scientific MA121315A647), mouse Alexa Fluor 568 (Thermo Fisher Scientific A-11031), or rabbit Alexa Fluor 647 (Thermo Fisher Scientific A-21244)]. Cells stained with Tomato Lectin were not incubated with additional secondary antibodies. Cells were incubated at 4 ºC for 30 mins. Cells were washed twice with PBS. For each sample, 10,000 cells were analyzed on a CytoFLEX Flow Cytometer (Beckman Coulter) and quantified with FlowJo (FlowJo). For each experiment, median fluorescence values for 3 biological replicates were compared to determine statistical significance.

### Primary Patient-Derived Cell Models

CCLF_MELM_0011_T melanoma tumor tissue and CCLF_PANC_0014_T pancreatic tumor tissue were obtained from Dana-Farber Cancer Institute hospital with informed consent and the cancer cell model line generation was approved by the ethical committee. Both tumor tissues were freshly received into the lab, rinsed with 95-100% ethanol very quickly and 1×PBS twice. Tissue was transferred to a sterile petri dish and the tissue was minced into small 1-2 mm fragments. Dissected tissues were dissociated in a collagenase/hyaluronidase (StemCell technologies 07912) medium for 1 hour. The red blood cells were further depleted by adding Ammonium Chloride Solution (StemCell technologies 07800). CCLF_MELM_0011_T dissociated cells were plated with smooth muscle growing medium-2 (Lonza CC-3181) into a six well plate, media was changed every 2-3 days and cells were split when a confluency of 80% was reached. A 1:3 ratio was used when splitting CCLF_MELM_0011_T. CCLF_PANC_0014_T dissociated cells were plated into a twenty-four well plate with a 50:50 mix of Clevers pancreas organoid media (Sato et al., 2011): Propagenix Conditioned media (Propagenix 256-100) and split when a confluency of 80% was reached. Media was changed every 3-4 days. A 1:2 ratio was used when splitting CCLF_PANC_0014_T which is a mixed population of suspension and adherent cells. Both lines were passaged 5 times before a pellet was taken for sequencing verification. The confirmed melanoma cell model and confirmed pancreatic adenocarcinoma cell model were propagated for another 10-15 passages and their cryovials preserved. CCLF_MELM_0011_T passage 11 cells and CCLF_PANC_0014_T passage 20 cells were used for this study.

Single-Sample Gene Set Enrichment Analysis (ssGSEA)

A total of 310 unique patient tumor transcriptomes that were collected prior to immunotherapy were used for ssGSEA (Auslander et al., 2018; Braun et al., 2020; Gide et al., 2019; Hugo et al., 2016; Pender et al., 2021; Riaz et al., 2017). As processed data was not available for the Gide et al. 2019 dataset (Gide et al., 2019), fastq files were downloaded and expression levels were estimated using RSEM v1.3.1 (Li and Dewey, 2011) as described below. Expression values for replicates from the same patient were averaged. ssGSEA (Barbie et al., 2009) as implemented by GSEAPY v0.10.4 was performed on each sample using default parameters to determine the normalized enrichment score of the 576 candidate genes. The z-score of the normalized enrichment scores was calculated on each dataset and aggregated. Patients were classified as responders (i.e., RECIST categories of complete response or partial response, clinical benefit, and no tumor progression) or non-responders (i.e., RECIST categories of stable disease or progressive disease, no clinical benefit, and tumor progression) based on the reported response to subsequent anti-PD-1 or anti-CTLA-4 checkpoint blockade therapy.

### Statistics

Statistical tests were applied with the sample size listed in the text and figure legends. Sample size represents the number of independent biological replicates. Data supporting main conclusions represents results from at least two independent experiments. All graphs with error bars report mean±s.e.m. values. Two-tailed 1-tests were performed unless otherwise indicated. Mantel-Cox log-rank tests were performed for host survival analyses. PRISM was used for basic statistical analysis and plotting (www.graphpad.com), and the R language and programming environment (www.r-project.org) was used for the remainder of the statistical analysis. Multiple hypothesis testing correction was applied where indicated.

### REFERENCES

Alderson, M. R., Smith, C. A., Tough, T. W., Davis-Smith, T., Armitage, R. J., Falk, B., Roux, E., Baker, E., Sutherland, G. R., and Din, W. S. (1994). Molecular and biological characterization of human 4-1BB and its ligand. Eur J Immunol 24, 2219-2227.
Auslander, N., Zhang, G., Lee, J. S., Frederick, D. T., Miao, B., Moll, T., Tian, T., Wei, Z., Madan, S., Sullivan, R. J., et al. (2018). Robust prediction of response to immune checkpoint blockade therapy in metastatic melanoma. Nat Med 24, 1545-1549.
Barbie, D. A., Tamayo, P., Boehm, J. S., Kim, S. Y., Moody, S. E., Dunn, I. F., Schinzel, A. C., Sandy, P., Meylan, E., Scholl, C., et al. (2009). Systematic RNA interference reveals that oncogenic KRAS-driven cancers require TBK1. Nature 462, 108-112.
Barretina, J., Caponigro, G., Stransky, N., Venkatesan, K., Margolin, A. A., Kim, S., Wilson, C. J., Lehar, J., Kryukov, G. V., Sonkin, D., et al. (2012). The Cancer Cell Line Encyclopedia enables predictive modelling of anticancer drug sensitivity. Nature 483, 603-607.
Braun, D. A., Hou, Y., Bakouny, Z., Ficial, M., Sant'Angelo, M., Forman, J., Ross-Macdonald, P., Berger, A. C., Jegede, O. A., Elagina, L., et al. (2020). Interplay of somatic alterations and immune infiltration modulates response to PD-1 blockade in advanced clear cell renal cell carcinoma. Nat Med 26, 909-918.
Chen, D. S., and Mellman, I. (2017). Elements of cancer immunity and the cancer-immune set point. Nature 541, 321-330.
Cho, J. H., Collins, J. J., and Wong, W. W. (2018). Universal Chimeric Antigen Receptors for Multiplexed and Logical Control of T Cell Responses. Cell 173, 1426-1438 e1411.
Czabotar, P. E., Lessene, G., Strasser, A., and Adams, J. M. (2014). Control of apoptosis by the BCL-2 protein family: implications for physiology and therapy. Nat Rev Mol Cell Biol 15, 49-63.
Decker, C. E., Young, T., Pasnikowski, E., Chiu, J., Song, H., Wei, Y., Thurston, G., and Daly, C. (2019). Genome-scale CRISPR activation screen uncovers tumor-intrinsic modulators of CD3 bispecific antibody efficacy. Sci Rep 9, 20068.
Doench, J. G. (2018). Am I ready for CRISPR? A user's guide to genetic screens. Nat Rev Genet 19, 67-80.
Dong, H., Strome, S. E., Salomao, D. R., Tamura, H., Hirano, F., Flies, D. B., Roche, P. C., Lu, J., Zhu, G., Tamada, K., et al. (2002). Tumor-associated B7-H1 promotes T-cell apoptosis: a potential mechanism of immune evasion. Nat Med 8, 793-800.
Dufva, O., Koski, J., Maliniemi, P., Ianevski, A., Klievink, J., Leitner, J., Polonen, P., Hohtari, H., Saeed, K., Hannunen, T., et al. (2020). Integrated drug profiling and CRISPR screening identify essential pathways for CAR T-cell cytotoxicity. Blood 135, 597-609.
Feng, J., Liu, T., Qin, B., Zhang, Y., and Liu, X. S. (2012). Identifying ChIP-seq enrichment using MACS. Nat Protoc 7, 1728-1740.
Garcia-Diaz, A., Shin, D. S., Moreno, B. H., Saco, J., Escuin-Ordinas, H., Rodriguez, G. A., Zaretsky, J. M., Sun, L., Hugo, W., Wang, X., et al. (2017). Interferon Receptor Signaling Pathways Regulating PD-L1 and PD-L2 Expression. Cell Rep 19, 1189-1201.
Gide, T. N., Quek, C., Menzies, A. M., Tasker, A. T., Shang, P., Holst, J., Madore, J., Lim, S. Y., Velickovic, R., Wongchenko, M., et al. (2019). Distinct Immune Cell Populations Define Response to Anti-PD-1 Monotherapy and Anti-PD-1/Anti-CTLA-4 Combined Therapy. Cancer Cell 35, 238-255 e236.Grossman, R. L., Heath, A. P., Ferretti, V., Varmus, H. E., Lowy, D. R., Kibbe, W. A., and Staudt, L. M. (2016). Toward a Shared Vision for Cancer Genomic Data. N Engl J Med 375, 1109-1112.
Grumont, R. J., Rourke, I. J., and Gerondakis, S. (1999). Rel-dependent induction of A1 transcription is required to protect B cells from antigen receptor ligation-induced apoptosis. Genes Dev 13, 400-411.
Hashiguchi, M., Kobori, H., Ritprajak, P., Kamimura, Y., Kozono, H., and Azuma, M. (2008). Triggering receptor expressed on myeloid cell-like transcript 2 (TLT-2) is a counter-receptor for B7-H3 and enhances T cell responses. Proc Natl Acad Sci USA 105, 10495-10500.
Hegde, P. S., and Chen, D. S. (2020). Top 10 Challenges in Cancer Immunotherapy. Immunity 52, 17-35.
Heinz, S., Benner, C., Spann, N., Bertolino, E., Lin, Y. C., Laslo, P., Cheng, J. X., Murre, C., Singh, H., and Glass, C. K. (2010). Simple combinations of lineage-determining transcription factors prime cis-regulatory elements required for macrophage and B cell identities. Mol Cell 38, 576-589.
Hoesel, B., and Schmid, J. A. (2013). The complexity of NF-kappaB signaling in inflammation and cancer. Mol Cancer 12, 86.
Holmgaard, R. B., Zamarin, D., Munn, D. H., Wolchok, J. D., and Allison, J. P. (2013). Indoleamine 2,3-dioxygenase is a critical resistance mechanism in antitumor T cell immunotherapy targeting CTLA-4. J Exp Med 210, 1389-1402.
Hugo, W., Zaretsky, J. M., Sun, L., Song, C., Moreno, B. H., Hu-Lieskovan, S., Berent-Maoz, B., Pang, J., Chmielowski, B., Cherry, G., et al. (2016). Genomic and Transcriptomic Features of Response to Anti-PD-1 Therapy in Metastatic Melanoma. Cell 165, 35-44.
Joung, J., Konermann, S., Gootenberg, J. S., Abudayyeh, O. O., Platt, R. J., Brigham, M. D., Sanjana, N. E., and Zhang, F. (2017). Genome-scale CRISPR-Cas9 knockout and transcriptional activation screening. Nat Protoc 12, 828-863.
Konermann, S., Brigham, M. D., Trevino, A. E., Joung, J., Abudayyeh, O. O., Barcena, C., Hsu, P. D., Habib, N., Gootenberg, J. S., Nishimasu, H., et al. (2015). Genome-scale transcriptional activation by an engineered CRISPR-Cas9 complex. Nature 517, 583-588.
Lawson, K. A., Sousa, C. M., Zhang, X., Kim, E., Akthar, R., Caumanns, J. J., Yao, Y., Mikolajewicz, N., Ross, C., Brown, K. R., et al. (2020). Functional genomic landscape of cancer-intrinsic evasion of killing by T cells. Nature.
Langmead, B., Trapnell, C., Pop, M., and Salzberg, S. L. (2009). Ultrafast and memory-efficient alignment of short DNA sequences to the human genome. Genome Biol 10, R25. Leitner, J., Klauser, C., Pickl, W. F., Stockl, J., Majdic, O., Bardet, A. F., Kreil, D. P., Dong, C., Yamazaki, T., Zlabinger, G., et al. (2009). B7-H3 is a potent inhibitor of human T-cell activation: No evidence for B7-H3 and TREML2 interaction. Eur J Immunol 39, 1754-1764.
Li, B., and Dewey, C. N. (2011). RSEM: accurate transcript quantification from RNA-Seq data with or without a reference genome. BMC Bioinformatics 12, 323.
Li, W., Xu, H., Xiao, T., Cong, L., Love, M. I., Zhang, F., Irizarry, R. A., Liu, J. S., Brown, M., and Liu, X. S. (2014). MAGeCK enables robust identification of essential genes from genome-scale CRISPR/Cas9 knockout screens. Genome Biol 15, 554.
Liu, T., Zhang, L., Joo, D., and Sun, S. C. (2017). NF-kappaB signaling in inflammation. Signal Transduct Target Ther 2.
Manguso, R. T., Pope, H. W., Zimmer, M. D., Brown, F. D., Yates, K. B., Miller, B. C., Collins, N. B., Bi, K., LaFleur, M. W., Juneja, V. R., et al. (2017). In vivo CRISPR screening identifies Ptpn2 as a cancer immunotherapy target. Nature 547, 413-418.
Nausch, N., Florin, L., Hartenstein, B., Angel, P., Schorpp-Kistner, M., and Cerwenka, A. (2006). Cutting edge: the AP-1 subunit JunB determines NK cell-mediated target cell killing by regulation of the NKG2D-ligand RAE-1epsilon. J Immunol 176, 7-11.
Pan, D., Kobayashi, A., Jiang, P., Ferrari de Andrade, L., Tay, R. E., Luoma, A. M., Tsoucas, D., Qiu, X., Lim, K., Rao, P., et al. (2018). A major chromatin regulator determines resistance of tumor cells to T cell-mediated killing. Science 359, 770-775.
Patel, S. J., Sanjana, N. E., Kishton, R. J., Eidizadeh, A., Vodnala, S. K., Cam, M., Gartner, J. J., Jia, L., Steinberg, S. M., Yamamoto, T. N., et al. (2017). Identification of essential genes for cancer immunotherapy. Nature 548, 537-542.
Pender, A., Titmuss, E., Pleasance, E. D., Fan, K. Y., Pearson, H., Brown, S. D., Grisdale, C. J., Topham, J. T., Shen, Y., Bonakdar, M., et al. (2021). Genome and Transcriptome Biomarkers of Response to Immune Checkpoint Inhibitors in Advanced Solid Tumors. Clin Cancer Res 27, 202-212.
Raudvere, U., Kolberg, L., Kuzmin, I., Arak, T., Adler, P., Peterson, H., and Vilo, J. (2019). g:Profiler: a web server for functional enrichment analysis and conversions of gene lists (2019 update). Nucleic Acids Res 47, W191-W198.
Reck, M., Rodriguez-Abreu, D., Robinson, A. G., Hui, R., Csoszi, T., Fulop, A., Gottfried, M., Peled, N., Tafreshi, A., Cuffe, S., et al. (2016). Pembrolizumab versus Chemotherapy for PD-L1-Positive Non-Small-Cell Lung Cancer. N Engl J Med 375, 1823-1833.
Riaz, N., Havel, J. J., Makarov, V., Desrichard, A., Urba, W. J., Sims, J. S., Hodi, F. S., Martin-Algarra, S., Mandal, R., Sharfman, W. H., et al. (2017). Tumor and Microenvironment Evolution during Immunotherapy with Nivolumab. Cell 171, 934-949 e916.
Robbins, P. F., Li, Y. F., El-Gamil, M., Zhao, Y., Wargo, J. A., Zheng, Z., Xu, H., Morgan, R. A., Feldman, S. A., Johnson, L. A., et al. (2008). Single and dual amino acid substitutions in TCR CDRs can enhance antigen-specific T cell functions. J Immunol 180, 6116-6131.
Rooney, M. S., Shukla, S. A., Wu, C. J., Getz, G., and Hacohen, N. (2015). Molecular and genetic properties of tumors associated with local immune cytolytic activity. Cell 160, 48-61.
Sato, T., Stange, D. E., Ferrante, M., Vries, R. G., Van Es, J. H., Van den Brink, S., Van Houdt, W. J., Pronk, A., Van Gorp, J., Siersema, P. D., et al. (2011). Long-term expansion of epithelial organoids from human colon, adenoma, adenocarcinoma, and Barrett's epithelium. Gastroenterology 141, 1762-1772.
Shaw, S., Luce, G. E., Quinones, R., Gress, R. E., Springer, T. A., and Sanders, M. E. (1986). Two antigen-independent adhesion pathways used by human cytotoxic T-cell clones. Nature 323, 262-264.
Sica, G. L., Choi, I. H., Zhu, G., Tamada, K., Wang, S. D., Tamura, H., Chapoval, A. I., Flies, D. B., Bajorath, J., and Chen, L. (2003). B7-H4, a molecule of the B7 family, negatively regulates T cell immunity. Immunity 18, 849-861.
Singh, N., Lee, Y. G., Shestova, O., Ravikumar, P., Hayer, K. E., Hong, S. J., Lu, X. M., Pajarillo, R., Agarwal, S., Kuramitsu, S., et al. (2020). Impaired Death Receptor Signaling in Leukemia Causes Antigen-Independent Resistance by Inducing CAR T-cell Dysfunction. Cancer Discov 10, 552-567.
Steinle, A., Li, P., Morris, D. L., Groh, V., Lanier, L. L., Strong, R. K., and Spies, T. (2001). Interactions of human NKG2D with its ligands MICA, MICB, and homologs of the mouse RAE-1 protein family. Immunogenetics 53, 279-287.
Stewart-Jones, G., Wadle, A., Hombach, A., Shenderov, E., Held, G., Fischer, E., Kleber, S., Nuber, N., Stenner-Liewen, F., Bauer, S., et al. (2009). Rational development of high-affinity T-cell receptor-like antibodies. Proc Natl Acad Sci USA 106, 5784-5788.
Tang, Z., Li, C., Kang, B., Gao, G., Li, C., and Zhang, Z. (2017). GEPIA: a web server for cancer and normal gene expression profiling and interactive analyses. Nucleic Acids Res 45, W98-W102.
Sun, L., Li, C. W., Chung, E. M., Yang, R., Kim, Y. S., Park, A. H., Lai, Y. J., Yang, Y., Wang, Y. H., Liu, J., et al. (2020). Targeting Glycosylated PD-1 Induces Potent Antitumor Immunity. Cancer Res 80, 2298-2310.
Togayachi, A., Kozono, Y., Kuno, A., Ohkura, T., Sato, T., Hirabayashi, J., Ikehara, Y., and Narimatsu, H. (2010). Beta3GnT2 (B3GNT2), a major polylactosamine synthase: analysis of B3GNT2-deficient mice. Methods Enzymol 479, 185-204.
Tseng, D., Volkmer, J. P., Willingham, S. B., Contreras-Trujillo, H., Fathman, J. W., Fernhoff, N. B., Seita, J., Inlay, M. A., Weiskopf, K., Miyanishi, M., et al. (2013). Anti-CD47 antibody-mediated phagocytosis of cancer by macrophages primes an effective antitumor T-cell response. Proc Natl Acad Sci USA 110, 11103-11108.
Tummers, B., Goedemans, R., Pelascini, L. P., Jordanova, E. S., van Esch, E. M., Meyers, C., Melief, C. J., Boer, J. M., and van der Burg, S. H. (2015). The interferon-related developmental regulator 1 is used by human papillomavirus to suppress NFkappaB activation. Nat Commun 6, 6537.
Vredevoogd, D. W., Kuilman, T., Ligtenberg, M. A., Boshuizen, J., Stecker, K. E., de Bruijn, B., Krijgsman, O., Huang, X., Kenski, J. C. N., Lacroix, R., et al. (2019). Augmenting Immunotherapy Impact by Lowering Tumor TNF Cytotoxicity Threshold. Cell 178, 585-599 e515.
Wan, Y. W., Allen, G. I., and Liu, Z. (2016). TCGA2STAT: simple TCGA data access for integrated statistical analysis in R. Bioinformatics 32, 952-954.
Wolchok, J. D., Kluger, H., Callahan, M. K., Postow, M. A., Rizvi, N. A., Lesokhin, A. M., Segal, N. H., Ariyan, C. E., Gordon, R. A., Reed, K., et al. (2013). Nivolumab plus ipilimumab in advanced melanoma. N Engl J Med 369, 122-133.
Xiang, W., Yang, C. Y., and Bai, L. (2018). MCL1 inhibition in cancer treatment. Onco Targets Ther 11, 7301-7314.
Zaretsky, J. M., Garcia-Diaz, A., Shin, D. S., Escuin-Ordinas, H., Hugo, W., Hu-Lieskovan, S., Torrejon, D. Y., Abril-Rodriguez, G., Sandoval, S., Barthly, L., et al. (2016). Mutations Associated with Acquired Resistance to PD-1 Blockade in Melanoma. N Engl J Med 375, 819-829.
Zong, W. X., Edelstein, L. C., Chen, C., Bash, J., and Gelinas, C. (1999). The prosurvival Bcl-2 homolog Bfl-1/A1 is a direct transcriptional target of NF-kappaB that blocks TNFalpha-induced apoptosis. Genes Dev 13, 382-387.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed:

1. A method of enhancing anti-tumor immunity in a subject in need thereof comprising administering to the subject one or more agents that reduce the expression or activity of B3GNT2.

2. The method of claim 1, wherein the one or more agents are small molecules that bind the active site of B3GNT2 or comprise an allosteric inhibitor of B3GNT2.

3. The method of claim 1, wherein the one or more agents is a gene editing system capable of reducing expression or activity of B3GNT2.

4. The method of claim 1, wherein the one more agents is a polynucleotide capable of inducing RNAi knock down of B3GNT2 expression.

5. A method of enhancing anti-tumor immunity in a subject in need thereof comprising administering to the subject one or more agents that inhibit poly-N-acetyllactosamine (poly-LacNAc) synthesis in tumor cells or reduces poly-LacNAc on surface N- and O-glycans.

6. The method of claim 5, wherein the one or more agents is a small molecule inhibitor of poly-LacNAc synthesis selected from the group consisting of benzyl-O—N-acetylgalactosamide (BAG), kifunensine (KIF), tunicamycin, 3′-Azidothymidine (AZT), 2-acetamido-1,3,6-tri-O-acetyl-4-deoxy-4-fluoro-D-glucopyranose [4-F-GlcNAc], and deoxymannojirimycin (DMN), whereby poly-LacNAc synthesis is inhibited.

7. The method of claim 5, wherein the one or more agents comprise an antibody that binds to a tumor specific surface marker and is linked to an enzyme capable of cleaving poly-LacNAc.

8. The method of claim 7, wherein the enzyme is selected from the group consisting of endo H, endo F2, endo F3, peptide:N-glycosidase F (PNGase F), endo D, O-glycosidase, endo-β-galactosidase, sialidase and O-sialoglycoprotease.

9. A method of enhancing anti-tumor immunity in a subject in need thereof comprising administering to the subject one or more agents capable of inhibiting the expression or activity of MCL1.

10. The method of claim 9, wherein the one or more agents increase the expression of one or more genes selected from the group consisting of BID, PMAIP1 (NOXA), BAX, BAK, BIM, BAD and BBC3 (PUMA).

11. The method of claim 9, wherein the one or more agents is a small molecule selected from the group consisting of Venetoclax, S63845, MIK665/S64315, AT101 (R-(−)-gossypol), TW-37, Gambogic acid, Sabutoclax (BI-97C1), Marinopyrrole A (maritoclax), UMI-77, A-1210477, Fesik's compounds, AMG176, AZD5991, Flavopiridol, Roscovitine, CR8, Voruciclib (P1446A-05), Cardiac glycoside, UNBS1450, Benzyl isothiocyanate, BAY43-9006, BAY1251152, BEZ235, AZD4573, AZD8055, SNS-032, dinaciclib, BEZ235, AZD8055, arsenic trioxide Bufalin, and analogues thereof.

12. The method of claim 9, wherein the one or more agents is a gene editing system capable of reducing MCL1 expression or activity.

13. The method of claim 9, wherein the one or more agents is a polynucleotide capable of inducing RNAi knock down of MCL1 expression.

14. A method of enhancing anti-tumor immunity in a subject in need thereof comprising administering to the subject one or more agents capable of inhibiting the expression or activity of BCL2A1.

15. The method of claim 14, wherein the one or more agents increase the expression of one or more genes selected from the group consisting of BID, PMAIP1 (NOXA), BAX, BAK, BIM, BAD and BBC3 (PUMA).

16. The method of claim 14, wherein the one or more agents is a small molecule selected from the group consisting of Venetoclax, AT101 (R-(−)-gossypol), TW-37, Gambogic acid, Sabutoclax (BI-97C1) and Marinopyrrole A (maritoclax).

17. The method of claim 14, wherein the one or more agents is a gene editing system capable of reducing BCL2A1 expression or activity.

18. The method of claim 14, wherein the one or more agents is a polynucleotide capable of inducing RNAi knock down of BCL2A1 expression.

19. A method of enhancing anti-tumor immunity in a subject in need thereof comprising administering to the subject one or more agents capable of inhibiting the expression or activity of JUNB.

20. The method of claim 19, wherein the one or more agents is a dominant-negative JUNB protein or vector encoding the same.

21. The method of claim 19, wherein the one or more agents is a small molecule selected from the group consisting of curcumin, SP100030, SPC-839, T-5224, K1115A, momordin I, isosteviol and analogues thereof.

22. The method of claim 19, wherein the one or more agents is gene editing system capable of reducing JUNB expression or activity.

23. The method of claim 19, wherein the one or more agents is a polynucleotide capable of inducing RNAi knock down of JUNB expression.

24. The method of claim 19, wherein the one or more agents is capable of inhibiting the expression or activity of one or more downstream targets of JUNB selected from Table 5 or Table 6.

25. A method of enhancing anti-tumor immunity in a subject in need thereof comprising administering to the subject one or more agents capable of inhibiting the expression or activity of one or more targets selected from Table 1 or Table 3.

26. An engineered T cell that expresses an enzyme capable of cleaving poly-LacNAc on the T cell surface.

27. The engineered T cell of claim 26, wherein the enzyme is selected from the group consisting of endo H, endo F2, endo F3, peptide:N-glycosidase F (PNGase F), endo D, O-glycosidase, endo-β-galactosidase, sialidase and O-sialoglycoprotease.

28. A method of enhancing anti-tumor immunity in a subject in need thereof comprising administering to the subject the engineered T cell of claim 26 or 27.

29. The method of any of claims 1 to 28, further comprising administering an immunotherapy in combination with the one or more agents or engineered T cell.

30. The method of claim 29, wherein the immunotherapy comprises adoptive cell transfer.

31. The method of claim 30, wherein adoptive cell transfer comprises the administration of CAR (chimeric antigen receptor) T cells or natural killer cells, T cells expressing a T cell receptor (TCR) specific for a tumor antigen, or tumor infiltrating lymphocytes (TILs).

32. The method of claim 29, wherein immunotherapy comprises checkpoint blockade (CPB) therapy.

33. The method of claim 32, wherein the checkpoint blockade therapy comprises anti-CTLA4, anti-PD-L1, anti-PD1, anti-TIM3, anti-TIGIT, anti-LAG3, or combinations thereof.

34. The method of any of claims 29 to 33, wherein the subject is treated with an immunotherapy followed by the one or more agents or engineered T cell.

35. The method of claim 34, wherein the subject is treated with an immunotherapy followed by the one or more agents or engineered T cell and an immunotherapy.

36. The method of any of claim 3, 11, 17, 22, or 25 wherein the gene editing system comprises a programmable nuclease.

37. The method of claim 36, wherein the programmable nuclease is selected from the group consisting of a CRISPR system, a zinc finger nuclease, a TALE, or a meganuclease.

38. The method of claim 36, wherein the gene editing system is a base editing system that introduces one or more base or base pair edits that reduce expression or activity of B3GNT2, MCL1, BCL2A1, or JUNB.

39. The method of claim 36, wherein the gene editing system is a prime editing system that introduces one or more base or base pair edits that reduce the expression or activity of B3GNT, MCL1, BCL2A1, or JUNB, or replaces all or a portion of the genomic sequence of B3GNT, MC1, BCL2A1, or JUNB with a non-functional variant.

40. The method of claim 36, wherein the gene editing system is a CAST system that replaces all or a portion of the genomic sequence of B3GNT, MC1, BCL2A1, or JUNB with a non-functional variant.

41. A method of monitoring the efficacy of an immunotherapy comprising detecting the expression of one or more genes selected from the group consisting of MCL1, BCL2A1, JUNB and B3GNT2 in a subject treated with an immunotherapy, wherein the subject is a non-responder to the immunotherapy if the one or more genes are upregulated after being treated.

42. The method of claim 41, wherein the expression is detected at two or more time points during treatment, wherein a trend of increasing expression of the one or more genes indicates a poor outcome and/or a non-responder.

43. The method of claim 41 or 42, further comprising administering a treatment according to any of claims 1 to 36, if a poor outcome and/or non-responder is indicated.

44. A method of treating a cancer in a subject in need thereof comprising
determining if the patient is an immunotherapy responder or non-responder by detecting in a tumor obtained from the subject the expression or activity of one or more genes selected from candidate genes in Table 2, wherein if the expression of the one or more genes is higher than a reference value the subject is an immunotherapy non-responder and if the one or more genes is lower than a reference value then the subject is an immunotherapy responder; and
if the subject is an immunotherapy non-responder, treating the subject using the method of any one of claims 1 to 40; and
if the subject is an immunotherapy responder, treating the subject with an immunotherapy.

45. The method of claim 44, wherein the one or more genes comprise B3GNT2, MCL1, BCL2A, JUNB, or a combination thereof.

46. A method of screening for agents capable of decreasing poly-LacNAc on tumor cells comprising:
a. contacting a population of tumor cells having upregulated B3GNT2 with an agent; and
b. detecting binding of one or more proteins to the tumor cells selected from the group consisting of CD2, 4-1BB, TREML2 (TLT2), NKG2D, and an antibody specific for an HLA class I bound tumor antigen, wherein increased binding indicates reduced poly-LacNAc.

47. The method of claim 46, wherein the one or more proteins are labeled with a detectable marker.
